(19) United States
(12) Patent Application Publication
(10) Pub. No.: US 2025/0195555 A1
(43) Pub. Date: Jun. 19, 2025

(54) TREATMENT OF HGFAC RELATED DISEASES AND DISORDERS

(71) Applicant: Empirico Inc., San Diego, CA (US)

(72) Inventors: Omri GOTTESMAN, San Diego, CA (US); Andrew ALLEN, San Diego, CA (US); Shannon BRUSE, San Diego, CA (US); Brian CAJES, San Diego, CA (US); David JAKUBOSKY, San Diego, CA (US); David LEWIS, Madison, WI (US); Gregory MCINNES, San Diego, CA (US); Jason O'ROURKE, San Diego, CA (US); David ROZEMA, Cross Plains, WI (US); Paul BUSKE, Middleton, CA (US)

(21) Appl. No.: 18/845,818

(22) PCT Filed: Mar. 16, 2023
(86) PCT No.: PCT/US2023/064565

§ 371 Date: Sep. 10, 2024

Related U.S. Application Data
(60) Provisional application No. 63/320,433, filed on Mar. 16, 2022.
(60) Provisional application No. 63/429,436, filed on Dec. 1, 2022.
(60) Provisional application No. 63/433,364, filed on Dec. 16, 2022.

(51) Int. Cl. A61K 31/712; A61P 35/00; C12N 15/10

(52) U.S. Cl. CPC A61K 31/712; A61P 35/00; C12N 15/102

(57) ABSTRACT

Disclosed herein are compositions comprising an oligonucleotide that targets HGFAC. The oligonucleotide may include a small interfering RNA (siRNA) or an antisense oligonucleotide (ASO). Also provided herein are methods of treating cancer that include providing an oligonucleotide that targets HGFAC in a subject.

### CROSS-REFERENCE

This application claims the benefit of U.S. Provisional Application No. 63/320,433, filed Mar. 16, 2022; U.S. Provisional Application No. 63/429,436, filed Dec. 1, 2022; and U.S. Provisional Application No. 63/433,364, filed Dec. 16, 2022, which applications are incorporated herein by reference.

### INCORPORATION BY REFERENCE OF SEQUENCE LISTING

The present application is being filed along with a Sequence Listing in electronic format. The Sequence Listing is provided as a file entitled 54462-738601.XML, created Mar. 16, 2023, which is 7,609,601 bytes in size. The information in the electronic format of the Sequence Listing is incorporated by reference in its entirety.

### BACKGROUND

Cancer is a serious threat, and improved therapeutics are needed.

### SUMMARY

Disclosed herein, in some embodiments, are compositions such as a composition comprising an oligonucleotide. The oligonucleotide may target HGFAC. Disclosed herein, in some embodiments, are compositions comprising an oligonucleotide that targets HGFAC and when administered to a subject having cancer in an effective amount improves a clinical response related to the cancer. In some embodiments, the improved clinical response comprises at least a 10% increase in a clinical response measurement relative to a baseline clinical response measurement obtained from the subject prior to administration of the composition. In some embodiments, the clinical response comprises progression free survival, duration of response, disease control rate, health-related quality of life, milestone survival, clinical benefit rate, pathological complete response, complete response, objective response rate, duration of clinical benefit, time to next treatment, time to treatment failure, disease-free survival, or time to cancer progression. Disclosed herein, in some embodiments, are compositions comprising an oligonucleotide that targets HGFAC and when administered to a subject in an effective amount alters an immune cell measurement in a subject. In some embodiments, the immune cell measurement is altered by about 10% or more, as compared to prior to administration. In some embodiments, the immune cell measurement comprises a myeloid derived suppressor cell or subpopulation count, CD8+ tumor infiltrating lymphocyte count, leukocyte count, T lymphocyte count, activated T lymphocyte count, B lymphocyte count, activated B lymphocyte count, monocyte count, macrophage count, activated macrophage count, dendritic cell count, neutrophil count, eosinophil count, basophil count, or mast cell count. Disclosed herein, in some embodiments, are compositions comprising an oligonucleotide that targets HGFAC and when administered to a subject in an effective amount increases an antibody level in a subject. In some embodiments, the antibody level is increased by about 10% or more, as compared to prior to administration. In some embodiments, the antibody level comprises an IgA level, IgG level, or IgM level. Disclosed herein, in some embodiments, are compositions comprising an oligonucleotide that targets HGFAC and when administered to a subject in an effective amount decreases a tumor marker level in a subject. In some embodiments, the tumor marker level is decreased by about 10% or more, as compared to prior to administration. In some embodiments, the tumor marker comprises CEA, PSA, CA 125, CA 15-3, CA 19-9, CA 27.29, CA 72-4, AFP, hCG, B2M, BTA, Calcitonin, CgA, CELLSEARCH, DCP, Gastrin, HE4, LDH, NSE, NMP22, or PAP. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises 1, 2, 3, 4, 5, 6, 7, 8, 9, 10, 11, 12, 13, 14, 15, 16, 17, 18, 19, 20, or 21 modified nucleosides. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the oligonucleotide comprises a sugar moiety attached at a 3′ or 5′ terminus of the oligonucleotide. In some embodiments, the sugar comprises N-acetylgalactosamine (GalNAc), N-acetylglucosamine (GlcNAc), or mannose. In some embodiments, the sugar moiety comprises a GalNAc moiety such as ETL17. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. Disclosed herein, in some embodiments, are compositions comprising an oligonucleotide that inhibits the expression of HGFAC, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 4803. In some embodiments, any one of the following is true with regard to the sense strand: (i) all purines comprise 2′ fluoro modified purines, and all pyrimidines comprise a mixture of 2′ fluoro and 2′-O-methyl modified pyrimidines; (ii) all purines comprise 2′-O-methyl modified purines, and all pyrimidines comprise a mixture of 2′ fluoro and 2′-O-methyl modified pyrimidines; (iii) all purines comprise 2′ fluoro modified purines, and all pyrimidines comprise 2′-O-methyl modified pyrimidines; (iv) all pyrimidines comprise 2′ fluoro modified pyrimidines, and all purines comprise a mixture of 2′ fluoro and 2′-O-methyl modified purines; (v) all pyrimidines comprise 2′-O-methyl modified pyrimidines, and all purines comprise a mixture of 2′ fluoro and 2′-O-methyl modified purines; or (vi) all pyrimidines comprise 2′ fluoro modified pyrimidines, and all purines comprise 2′-O-methyl modified purines. In some embodiments, the sense strand comprises any one of modification patterns 1S, 2S, 3S, 4S, 5S, 6S, 7S, 8S, or 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S. In some embodiments, any one of the following is true with regard to the antisense strand: (i) all purines comprise 2′ fluoro modified purines, and all pyrimidines comprise a mixture of 2′ fluoro and 2′-O-methyl modified pyrimidines; (ii) all purines comprise 2′-O-methyl modified purines, and all pyrimidines comprise a mixture of 2′ fluoro and 2′-O-methyl modified pyrimidines; (iii) all purines comprise 2′-O-methyl modified purines, and all pyrimidines comprise 2′ fluoro modified pyrimidines; (iv) all pyrimidines comprise 2′ fluoro modified pyrimidines, and all purines comprise a mixture of 2′ fluoro and 2′-O-methyl modified purines; (v) all pyrimidines comprise 2′-O-methyl modified pyrimidines, and all purines comprise a mixture of 2′ fluoro and 2′-O-methyl modified purines; or (vi) all pyrimidines comprise 2′-O-methyl modified pyrimidines, and all purines comprise 2′ fluoro modified purines. In some embodiments, the antisense strand comprises any one of modification patterns 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises the nucleic acid sequence of any one of SEQ ID NOs: 1-2051, 4562-4616, or 4757-4779, and the antisense strand comprises the nucleic acid sequence of any one of SEQ ID NOs: 2052-4102, 4617-4671, or 4780-4782. In some embodiments, the oligonucleotide comprises an antisense oligonucleotide (ASO). In some embodiments, the ASO is 12-30 nucleosides in length. Disclosed herein, in some embodiments, are compositions comprising an oligonucleotide that inhibits the expression of HGFAC, wherein the oligonucleotide comprises an ASO about 12-30 nucleosides in length and a nucleoside sequence complementary to about 12-30 contiguous nucleosides of SEQ ID NO: 4803. In some embodiments, the composition comprises a pharmaceutically acceptable carrier. Disclosed herein, in some embodiments, are methods of treatment. The method may include treatment of cancer in a subject in need.

The method may include administering a composition such as a composition comprising an oligonucleotide that targets HGFAC. Disclosed herein, in some embodiments, are methods of treating a subject having cancer, comprising administering an effective amount of the composition to the subject.

Some embodiments include administering a checkpoint inhibitor to the subject. Some embodiments include administering radiotherapy to the subject. In some embodiments, the cancer comprises a malignant neoplasm, a solid tumor, or a hematological cancer. In some embodiments, the cancer comprises a malignant neoplasm of a urinary tract, malignant neoplasm of an endocrine gland, malignant neoplasm of a soft tissue, malignant neoplasm of skin, malignant neoplasm of a skeletal system, malignant neoplasm of a respiratory organ, malignant neoplasm of an intrathoracic organ, malignant neoplasm of a genital organ, malignant neoplasm of a lip, malignant neoplasm of an oral cavity, malignant neoplasm of a pharynx, malignant neoplasm of an eye, malignant neoplasm of a central nervous system, malignant neoplasm of a brain, malignant neoplasm of a digestive system, malignant neoplasm of a breast, malignant neoplasm of a pancreas, or a malignant melanoma.

### BRIEF DESCRIPTION OF THE DRAWINGS

FIG. 1A depicts expression of HGFAC mRNA in transfected RPE cells.

FIG. 1B depicts expression of intracellular HGFAC protein expression from whole cell lysates by western blot.

FIG. 1C quantifies the fold change of HGFAC expression in difference cell variants.

FIG. 1D depicts the levels of secreted HGFAC in cells transfected with wildtype or each variant.

### DETAILED DESCRIPTION OF THE INVENTION

Large-scale human genetic data can improve the success rate of pharmaceutical discovery and development. A Genome Wide Association Study (GWAS) detects associations between genetic variants and traits in a population sample, and this improves understanding of the biology of disease and provides evidence of applicable treatments. A GWAS generally utilizes genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is considered associated with disease. Association statistics used in a GWAS include p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation is used to identify a causal genetic variant identified via GWAS, and in many cases leads to identification of disease-causing genes. In particular, understanding the functional effect of a causal genetic variant (for example, loss of protein function, gain of protein function, increase in gene expression, or decrease in gene expression) allows that variant to be used as a proxy for therapeutic modulation of the target gene, or to gain insight into potential therapeutic efficacy and safety of a therapeutic that modulates that target.

Identification of such gene-disease associations has provided insights into disease biology and is used to identify novel therapeutic targets for the pharmaceutical industry. In order to translate the therapeutic insights derived from human genetics, disease biology in patients is exogenously ‘programmed’ into replicating the observation from human genetics. There are several options for therapeutic modalities that may be brought to bear in translating therapeutic targets identified via human genetics into novel medicines. These include well established therapeutic modalities such as small molecules and monoclonal antibodies, maturing modalities such as oligonucleotides, and emerging modalities such as gene therapy and gene editing. The choice of therapeutic modality depends on factors such as the location of a target (for example, intracellular, extracellular, or secreted), a relevant tissue (for example, liver) and a relevant indication.

The HGFAC gene is located on chromosome 4, and encodes hepatocyte growth factor activator (HGFAC). HGFAC may include 655 amino acids or have a mass of about 70.7 kDa. HGFAC may be expressed in liver cells. HGFAC can act as a serine protease that converts hepatocyte growth factor to active form. HGFAC may be secreted, for example, into the bloodstream. HGFAC may be part of the peptidase S1 protein family. An example of an HGFAC amino acid sequence, and further description of HGFAC is included at uniprot.org under accession no. Q04756 (last modified Feb. 23, 2022).

Here it is shown that deleterious gene variants of HGFAC result in protection from malignant neoplasms. Therefore, inhibition of HGFAC may serve as a therapeutic for treatment of a variety of cancers. Disclosed herein are compositions comprising an oligonucleotide that targets HGFAC. Where inhibition or targeting of HGFAC is disclosed, it is contemplated that some embodiments may include inhibiting or targeting a HGFAC protein or HGFAC RNA. For example, by inhibiting or targeting an RNA (e.g., mRNA) encoded by the HGFAC gene using an oligonucleotide described herein, the HGFACE protein may be inhibited or targeted as a result of there being less production of the HGFAC protein by translation of the HGFAC RNA; or a HGFAC protein may be targeted or inhibited by an oligonucleotide that binds or interacts with a HGFAC RNA and reduces production of the HGFAC protein from the HGFAC RNA. Thus, targeting HGFAC may refer to binding a HGFAC RNA and reducing HGFAC RNA or protein levels. The oligonucleotide may include a small interfering RNA (siRNA) or an antisense oligonucleotide (ASO). Also provided herein are methods of treating cancer by providing an oligonucleotide that targets HGFAC to a subject in need thereof.

### I. Compositions

Disclosed herein, in some embodiments, are compositions comprising a therapeutic modality that targets or inhibits HGFAC. Non-limiting examples are listed in Table 1B. In some embodiments, a therapeutic modality, composition, or compound described herein may refer to any one of: a dsRNA agent (e.g., siRNA), antisense oligonucleotide, and a small molecule compound. In some embodiments, the composition comprises a therapeutic modality that targets HGFAC. In some embodiments, the composition consists of therapeutic modality that targets HGFAC. In some embodiments, the composition comprises an antibody or a binding fragment thereof. In some embodiments, the therapeutic modality reduces HGFAC mRNA expression in the subject. In some embodiments, the therapeutic modality reduces HGFAC protein expression in the subject. In some embodiments, a composition described herein is used in a method of treating a disorder in a subject in need thereof. Some embodiments relate to a composition for use in a method of treating a disorder such as cancer. Some embodiments relate to use of a composition, in a method of treating a disorder such as cancer.

TABLE 1A
Therapeutic modalities

| Type of Therapy | Description or Examples | Possible Target | Possible Mode of Action |
|---|---|---|---|
| Antisense Oligonucleotide (ASO) | Single-stranded DNA or RNA | mRNA | Prevents translation or splicing |
| Small Interfering RNA (siRNA) | Double-stranded RNA | mRNA | Induces innate gene-silencing causing target degradation |
| Anti-Micro RNA | Single-stranded RNA | microRNA | Inactivates microRNA, affecting expression |
| Micro-RNA Mimic | Double-stranded RNA | mRNA | Prevents translation |
| mRNA Analog | Single-stranded RNA | Ribosomes | Induces translation of novel protein |
| Aptamer | Single-stranded DNA or RNA | Many | Inactivates or modifies target |
| CRISPR | gRNA and Cas9 enzyme | DNA | Inactivates gene or alters DNA sequence |
| Small Molecule Inhibitor | Low molecular weight organic | Protein | Inhibits a specific function of a protein or disrupts |
| | compound | | protein-protein interactions |
| Antibody | Polyclonal antibody, monoclonal | Protein | Inhibits a specific function of a protein or disrupts |
| | antibody, nanobody, antibody | | protein-protein interactions |
| | binding fragment | | |

Because HGFAC is a protease, some small molecule inhibitors may be useful for inhibiting its protease activity. Some protease inhibitors may have sub-optimal IC50 values for HGFAC relative to other protein targets, or may be non-specific for HGFAC, though, so other therapeutic modalities such as antibodies or oligonucleotide therapeutics may be more useful. Nafamostat may inhibit HGFAC with an IC50 of about 150 nM for HGFAC. Nafamostat may inhibit other serine proteases with a much lower IC50 than HGFAC. In some embodiments, the small molecule is a protease inhibitor such as a serine protease inhibitor. An example of a serine serine protease inhibitor may include Nafamostat.

Disclosed herein, in some embodiments, are compositions comprising an oligonucleotide. In some embodiments, the composition comprises an oligonucleotide that targets HGFAC. In some embodiments, the composition consists of an oligonucleotide that targets HGFAC. In some embodiments, the oligonucleotide reduces HGFAC mRNA expression in the subject. In some embodiments, the oligonucleotide reduces HGFAC protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder such as cancer. Some embodiments relate to use of a composition comprising an oligonucleotide, in a method of treating a disorder such as cancer.

Some embodiments include a composition comprising an oligonucleotide that targets HGFAC and when administered to a subject in an effective amount decreases HGFAC mRNA or protein levels in a cell, fluid, or tissue. In some embodiments, the composition comprises an oligonucleotide that targets HGFAC and when administered to a subject in an effective amount decreases HGFAC mRNA levels in a cell or tissue. In some embodiments, the cell is a liver cell or hepatocyte. In some embodiments, the tissue is liver tissue. In some embodiments, the HGFAC mRNA levels are decreased by about 2.5% or more, about 5% or more, or about 7.5% or more, as compared to prior to administration. In some embodiments, the HGFAC mRNA levels are decreased by about 10% or more, as compared to prior to administration. In some embodiments, the HGFAC mRNA levels are decreased by about 20% or more, about 30% or more, about 40% or more, about 50% or more, about 60% or more, about 70% or more, about 80% or more, about 90% or more, or about 100%, as compared to prior to administration. In some embodiments, the HGFAC mRNA levels are decreased by no more than about 2.5%, no more than about 5%, or no more than about 7.5%, as compared to prior to administration. In some embodiments, the HGFAC mRNA levels are decreased by no more than about 10%, as compared to prior to administration. In some embodiments, the HGFAC mRNA levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the HGFAC mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets HGFAC and when administered to a subject in an effective amount decreases HGFAC protein levels in a cell, fluid, or tissue. In some embodiments, the cell is a liver cell or hepatocyte. In some embodiments, the fluid is a serum, blood, or plasma. In some embodiments, the tissue is liver tissue. In some embodiments, the HGFAC protein levels are decreased by about 2.5% or more, about 5% or more, or about 7.5% or more, as compared to prior to administration. In some embodiments, the HGFAC protein levels are decreased by about 10% or more, as compared to prior to administration. In some embodiments, the HGFAC protein levels are decreased by about 20% or more, about 30% or more, about 40% or more, about 50% or more, about 60% or more, about 70% or more, about 80% or more, about 90% or more, or about 100%, as compared to prior to administration. In some embodiments, the HGFAC protein levels are decreased by no more than about 2.5%, no more than about 5%, or no more than about 7.5%, as compared to prior to administration. In some embodiments, the HGFAC protein levels are decreased by no more than about 10%, as compared to prior to administration. In some embodiments, the HGFAC protein levels are decreased by no more than about 20%, no more than about 30%, no more than about 40%, no more than about 50%, no more than about 60%, no more than about 70%, no more than about 80%, or no more than about 90%, as compared to prior to administration. In some embodiments, the HGFAC protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets HGFAC and when administered to a subject in an effective amount diminishes a cancer phenotype. The cancer may include: malignant neoplasms, solid tumors, hematological cancers, malignant neoplasms of urinary tract, malignant neoplasms of thyroid and other endocrine glands, malignant neoplasms of soft tissue, malignant neoplasms of skin, malignant neoplasms of skeletal system, malignant neoplasms of respiratory and intrathoracic organs, malignant neoplasms of male genital organs, malignant neoplasms of female genital organs, malignant neoplasms of lip, oral cavity and pharynx, malignant neoplasms of eye, brain and other parts of central nervous system, malignant neoplasms of digestive system, malignant neoplasms of breast, malignant neoplasms of pancreas, or malignant melanoma. In some embodiments, the cancer phenotype is decreased by about 2.5% or more, about 5% or more, or about 7.5% or more, as compared to prior to administration. In some embodiments, the cancer phenotype is decreased by about 10% or more, as compared to prior to administration. In some embodiments, the cancer phenotype is decreased by about 20% or more, about 30% or more, about 40% or more, about 50% or more, about 60% or more, about 70% or more, about 80% or more, about 90% or more, or about 100%, as compared to prior to administration. In some embodiments, the cancer phenotype is decreased by no more than about 2.5%, no more than about 5%, or no more than about 7.5%, as compared to prior to administration. In some embodiments, the cancer phenotype is decreased by no more than about 10%, as compared to prior to administration. In some embodiments, the cancer phenotype is decreased by no more than about 20%, no more than about 30%, no more than about 40%, no more than about 50%, no more than about 60%, no more than about 70%, no more than about 80%, or no more than about 90%, as compared to prior to administration. In some embodiments, the cancer phenotype is decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets HGFAC and when administered to a subject in an effective amount enhances a protective phenotype against a cancer in the subject. An example of a protective phenotype may include an anti-cancer immune response. The cancer may include: malignant neoplasms, solid tumors, hematological cancers, malignant neoplasms of urinary tract, malignant neoplasms of thyroid and other endocrine glands, malignant neoplasms of soft tissue, malignant neoplasms of skin, malignant neoplasms of skeletal system, malignant neoplasms of respiratory and intrathoracic organs, malignant neoplasms of male genital organs, malignant neoplasms of female genital organs, malignant neoplasms of lip, oral cavity and pharynx, malignant neoplasms of eye, brain and other parts of central nervous system, malignant neoplasms of digestive system, malignant neoplasms of breast, malignant neoplasms of pancreas, or malignant melanoma. In some embodiments, the protective phenotype is increased by about 2.5% or more, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets HGFAC and when administered to a subject in an effective amount increases or improves a clinical response in the subject. The clinical response may include: immune specific related response criteria (irRC) such as set forth in iRECIST, progression free survival (PFS), duration of response (DOR), disease control rate (DCR), health-related quality of life, milestone survival, clinical benefit rate, pathological complete response, complete response, objective response rate, duration of clinical benefit, time to next treatment, time to treatment failure, disease-free survival, or time to progression. In some embodiments, the clinical response is increased by about 2.5% or more, about 5% or more, or about 7.5% or more, as compared to prior to administration. In some embodiments, the clinical response is increased by about 10% or more, as compared to prior to administration. In some embodiments, the clinical response is increased by about 20% or more, about 30% or more, about 40% or more, about 50% or more, about 60% or more, about 70% or more, about 80% or more, about 90% or more, or about 100% or more, as compared to prior to administration. In some embodiments, the clinical response is increased by about 200% or more, about 300% or more, about 400% or more, about 500% or more, about 600% or more, about 700% or more, about 800% or more, about 900% or more, or about 1000% or more, as compared to prior to administration. In some embodiments, the clinical response is increased by no more than about 2.5%, no more than about 5%, or no more than about 7.5%, as compared to prior to administration. In some embodiments, the clinical response is increased by no more than about 10%, as compared to prior to administration. In some embodiments, the clinical response is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the clinical response is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the clinical response is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets HGFAC and when administered to a subject in an effective amount improves a cell count measurement in the subject. The cell count measurement may be an immune cell count measurement. The cell count measurement may be indicative of an anti-cancer immune response. The cell count measurement may include a cancer cell count measurement. The improvement may comprise a change. In some embodiments, the change is an increase. in some embodiments, the change is a decrease (e.g. a cancer cell count). The cell count measurement may include: myeloid derived suppressor cell (MDSC) counts and subpopulations, CD8+ tumor infiltrating lymphocytes (TILs), leukocyte counts, T lymphocyte counts, T lymphocyte activation states, B lymphocyte counts, B lymphocyte activation states, monocyte counts, macrophage counts, macrophage activation states, dendritic cell counts, neutrophil counts, eosinophil counts, basophil counts, or mast cell counts. In some embodiments, cell count measurement is improved by about 2.5% or more, about 5% or more, or about 7.5% or more, as compared to prior to administration. In some embodiments, the cell count measurement is improved by about 10% or more, as compared to prior to administration. In some embodiments, the cell count measurement is improved by about 20% or more, about 30% or more, about 40% or more, about 50% or more, about 60% or more, about 70% or more, about 80% or more, about 90% or more, or about 100%, as compared to prior to administration. In some embodiments, the cell count measurement is improved by no more than about 2.5%, no more than about 5%, or no more than about 7.5%, as compared to prior to administration. In some embodiments, the cell count measurement is improved by no more than about 10%, as compared to prior to administration. In some embodiments, the cell count measurement is improved by no more than about 20%, no more than about 30%, no more than about 40%, no more than about 50%, no more than about 60%, no more than about 70%, no more than about 80%, or no more than about 90%, as compared to prior to administration. In some embodiments, the cell count measurement is improved by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned. In some embodiments where the improvement is an increase, the change is by more than 100%.

In some embodiments, the composition comprises an oligonucleotide that targets HGFAC and when administered to a subject in an effective amount improves antibody levels in the subject. The antibody levels may be indicative of an anti-cancer immune response. The improvement may comprise a change. In some embodiments, the change is an increase. in some embodiments, the change is a decrease.

The antibody levels may include: IgA levels, IgG levels, or IgM levels. In some embodiments, the antibody levels are improved by about 2.5% or more, about 5% or more, or about 7.5% or more, as compared to prior to administration. In some embodiments, the antibody levels are improved by about 10% or more, as compared to prior to administration. In some embodiments, the antibody levels are improved by about 20% or more, about 30% or more, about 40% or more, about 50% or more, about 60% or more, about 70% or more, about 80% or more, about 90% or more, or about 100%, as compared to prior to administration. In some embodiments, the antibody levels are improved by no more than about 2.5%, no more than about 5%, or no more than about 7.5%, as compared to prior to administration. In some embodiments, the antibody levels are improved by no more than about 10%, as compared to prior to administration. In some embodiments, the antibody levels are improved by no more than about 20%, no more than about 30%, no more than about 40%, no more than about 50%, no more than about 60%, no more than about 70%, no more than about 80%, or no more than about 90%, as compared to prior to administration. In some embodiments, the antibody levels are improved by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages. In some embodiments where the improvement is an increase, the change is by more than 100%.

In some embodiments, the composition comprises an oligonucleotide that targets HGFAC and when administered to a subject in an effective amount improves tumor marker levels in the subject. The improvement may comprise a change. In some embodiments, the change is an increase. in some embodiments, the change is a decrease. The tumor marker levels may include levels of tumor markers such as CEA, PSA, CA 125, CA 15-3, CA 19-9, CA 27.29, CA 72-4, AFP, hCG, B2M, BTA, Calcitonin, CgA, CELLSEARCH, DCP, Gastrin, HE4, LDH, NSE, NMP22, or PAP. In some embodiments, the tumor marker levels are improved by about 2.5% or more, about 5% or more, or about 7.5% or more, as compared to prior to administration. In some embodiments, the tumor marker levels are improved by about 10% or more, as compared to prior to administration. In some embodiments, the tumor marker levels are improved by about 20% or more, about 30% or more, about 40% or more, about 50% or more, about 60% or more, about 70% or more, about 80% or more, about 90% or more, or about 100%, as compared to prior to administration. In some embodiments, the tumor marker levels are improved by no more than about 2.5%, no more than about 5%, or no more than about 7.5%, as compared to prior to administration. In some embodiments, the tumor marker levels are improved by no more than about 10%, as compared to prior to administration. In some embodiments, the tumor marker levels are improved by no more than about 20%, no more than about 30%, no more than about 40%, no more than about 50%, no more than about 60%, no more than about 70%, no more than about 80%, or no more than about 90%, as compared to prior to administration. In some embodiments, the tumor marker levels are improved by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages. In some embodiments where the improvement is an increase, the change is by more than 100%.

A. siRNAs

In some embodiments, the composition comprises an oligonucleotide that targets HGFAC, wherein the oligonucleotide comprises a small interfering RNA (siRNA). In some embodiments, the composition comprises an oligonucleotide that targets HGFAC, wherein the oligonucleotide comprises a small interfering RNA (siRNA) comprising a sense strand and an antisense strand.

In some embodiments, the composition comprises an oligonucleotide that inhibits the expression of HGFAC, wherein the oligonucleotide comprises an siRNA comprising a sense strand and an antisense strand, wherein the sense strand is 12-30 nucleosides in length. In some embodiments, the composition comprises a sense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The sense strand may be 14-30 nucleosides in length. In some embodiments, the composition comprises an antisense strand is 12-30 nucleosides in length. In some embodiments, the composition comprises an antisense strand that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. The antisense strand may be 14-30 nucleosides in length.

In some embodiments, the composition comprises an oligonucleotide that inhibits the expression of HGFAC, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of a full-length human HGFAC mRNA sequence such as SEQ ID NO: 4803. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 4803.

In some embodiments, the composition comprises an oligonucleotide that inhibits the expression of HGFAC, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of HGFAC, wherein the oligonucleotide comprises an siRNA comprising a sense strand and an antisense strand, wherein the siRNA binds with a 19mer in a human HGFAC mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human HGFAC mRNA.

In some embodiments, the composition comprises an oligonucleotide that inhibits the expression of HGFAC, wherein the oligonucleotide comprises an siRNA comprising a sense strand and an antisense strand, wherein the siRNA binds with a 17mer in a non-human primate HGFAC mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate HGFAC mRNA.

In some embodiments, the composition comprises an oligonucleotide that inhibits the expression of HGFAC, wherein the oligonucleotide comprises an siRNA comprising a sense strand and an antisense strand, wherein the siRNA binds with a human HGFAC mRNA and less than or equal to 20 human off-targets, with no more than 2 mismatches in the antisense strand. In some embodiments, the siRNA binds with a human HGFAC mRNA and less than or equal to 10 human off-targets, with no more than 2 mismatches in the antisense strand. In some embodiments, the siRNA binds with a human HGFAC mRNA and less than or equal to 30 human off-targets, with no more than 2 mismatches in the antisense strand. In some embodiments, the siRNA binds with a human HGFAC mRNA and less than or equal to 40 human off-targets, with no more than 2 mismatches in the antisense strand. In some embodiments, the siRNA binds with a human HGFAC mRNA and less than or equal to 50 human off-targets, with no more than 2 mismatches in the antisense strand. In some embodiments, the siRNA binds with a human HGFAC mRNA and less than or equal to 10 human off-targets, with no more than 3 mismatches in the antisense strand. In some embodiments, the siRNA binds with a human HGFAC mRNA and less than or equal to 20 human off-targets, with no more than 3 mismatches in the antisense strand. In some embodiments, the siRNA binds with a human HGFAC mRNA and less than or equal to 30 human off-targets, with no more than 3 mismatches in the antisense strand. In some embodiments, the siRNA binds with a human HGFAC mRNA and less than or equal to 40 human off-targets, with no more than 3 mismatches in the antisense strand. In some embodiments, the siRNA binds with a human HGFAC mRNA and less than or equal to 50 human off-targets, with no more than 3 mismatches in the antisense strand.

In some embodiments, the composition comprises an oligonucleotide that inhibits the expression of HGFAC, wherein the oligonucleotide comprises an siRNA comprising a sense strand and an antisense strand, siRNA binds with a human HGFAC mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 1-2051, 4562-4616, or 4757-4779, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-2051, 4562-4616, or 4757-4779, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-2051, 4562-4616, or 4757-4779, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 1-2051, 4562-4616, or 4757-4779.

In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 1-2051, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 1-2051, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 1-2051, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 1-2051.

In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 4562-4616, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 4562-4616, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 4562-4616, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 4562-4616.

In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 4757-4779, or a nucleic acid sequence thereof having 1 or 2 nucleoside substitutions, additions, or deletions. In some embodiments, the sense strand comprises a nucleoside sequence comprising or consisting of the sequence of any one of SEQ ID NOs: 4757-4779, or a nucleic acid sequence thereof having 3 or 4 nucleoside substitutions, additions, or deletions. In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the sense strand comprises a nucleoside sequence comprising or consisting of the sequence of any one of SEQ ID NOs: 4757-4779, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the sense strand comprises a nucleoside sequence comprising or consisting of the sequence of any one of SEQ ID NOs: 4757-4779.

In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 2052-4102, 4617-4671, or 4780-4782, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 2052-4102, 4617-4671, or 4780-4782, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 2052-4102, 4617-4671, or 4780-4782, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 2052-4102, 4617-4671, or 4780-4782.

In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 2052-4102, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 2052-4102, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 2052-4102, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 2052-4102.

In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 4617-4671, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 4617-4671, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 4617-4671, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 4617-4671.

In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 4780-4782, or a nucleic acid sequence thereof having 1 or 2 nucleoside substitutions, additions, or deletions. In some embodiments, the antisense strand sequence comprises a nucleoside sequence comprising or consisting of the sequence of any one of SEQ ID NOs: 4780-4782, or a nucleic acid sequence thereof having 3 or 4 nucleoside substitutions, additions, or deletions. In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides. In some embodiments, the antisense strand comprises a nucleoside sequence comprising or consisting of the sequence of any one of SEQ ID NOs: 4780-4782, or a nucleic acid sequence thereof having 1 or 2 nucleoside additions at the 3′ end. In some embodiments, the composition comprises an oligonucleotide that inhibits the expression of HGFAC, wherein the oligonucleotide comprises an siRNA comprising a sense strand and an antisense strand, wherein the antisense strand comprises a nucleoside sequence comprising or consisting of the sequence of any one of SEQ ID NOs: 4780-4782.

In some embodiments, the siRNA comprises the sense strand and/or the antisense strand sequence of an siRNA in any one of Tables A-E, G, H, 5, 14 or a nucleic acid sequence thereof having 3 or 4 nucleoside substitutions, additions, or deletions. In some embodiments, the siRNA comprises the sense strand and/or the antisense strand sequence of an siRNA in any one of Tables A-E, G, H, 5, 14 or a nucleic acid sequence thereof having 1 or 2 nucleoside substitutions, additions, or deletions. In some embodiments, the siRNA comprises the sense strand and/or the antisense strand sequence of an siRNA in any one of Tables A-E, G, H, 5, 14. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset A, or a nucleic acid sequence thereof having 3 or 4 nucleoside substitutions, additions, or deletions. In some embodiments, the siRNA comprises the sense strand and/or the antisense strand sequence of an siRNA of subset A, or a nucleic acid sequence thereof having 1 or 2 nucleoside substitutions, additions, or deletions. In some embodiments, the siRNA comprises the sense strand and/or the antisense strand sequence of an siRNA of subset A. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset B, or a nucleic acid sequence thereof having 3 or 4 nucleoside substitutions, additions, or deletions. In some embodiments, the siRNA comprises the sense strand and/or the antisense strand sequence of an siRNA of subset B, or a nucleic acid sequence thereof having 1 or 2 nucleoside substitutions, additions, or deletions. In some embodiments, the siRNA comprises the sense strand and/or the antisense strand sequence of an siRNA of subset B. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset C, or a nucleic acid sequence thereof having 3 or 4 nucleoside substitutions, additions, or deletions. In some embodiments, the siRNA comprises the sense strand and/or the antisense strand sequence of an siRNA of subset C, or a nucleic acid sequence thereof having 1 or 2 nucleoside substitutions, additions, or deletions. In some embodiments, the siRNA comprises the sense strand and/or the antisense strand sequence of an siRNA of subset C. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset D, or a nucleic acid sequence thereof having 3 or 4 nucleoside substitutions, additions, or deletions. In some embodiments, the siRNA comprises the sense strand and/or the antisense strand sequence of an siRNA of subset D, or a nucleic acid sequence thereof having 1 or 2 nucleoside substitutions, additions, or deletions. In some embodiments, the siRNA comprises the sense strand and/or the antisense strand sequence of an siRNA of subset D. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset E, or a nucleic acid sequence thereof having 3 or 4 nucleoside substitutions, additions, or deletions. In some embodiments, the siRNA comprises the sense strand and/or the antisense strand sequence of an siRNA of subset E, or a nucleic acid sequence thereof having 1 or 2 nucleoside substitutions, additions, or deletions. In some embodiments, the siRNA comprises the sense strand and/or the antisense strand sequence of an siRNA of subset E. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset G, or a nucleic acid sequence thereof having 3 or 4 nucleoside substitutions, additions, or deletions. In some embodiments, the siRNA comprises the sense strand and/or the antisense strand sequence of an siRNA of subset G, or a nucleic acid sequence thereof having 1 or 2 nucleoside substitutions, additions, or deletions. In some embodiments, the siRNA comprises the sense strand and/or the antisense strand sequence of an siRNA of subset G. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of subset H, or a nucleic acid sequence thereof having 3 or 4 nucleoside substitutions, additions, or deletions. In some embodiments, the siRNA comprises the sense strand and/or the antisense strand sequence of an siRNA of subset H, or a nucleic acid sequence thereof having 1 or 2 nucleoside substitutions, additions, or deletions. In some embodiments, the siRNA comprises the sense strand and/or the antisense strand sequence of an siRNA of subset H. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA of Table 5, or a nucleic acid sequence thereof having 3 or 4 nucleoside substitutions, additions, or deletions. In some embodiments, the siRNA comprises the sense strand and/or the antisense strand sequence of an siRNA of Table 5, or a nucleic acid sequence thereof having 1 or 2 nucleoside substitutions, additions, or deletions. In some embodiments, the siRNA comprises the sense strand and/or the antisense strand sequence of an siRNA of Table 5. In some embodiments, the siRNA is cross-reactive with a non-human primate (NHP) HGFAC mRNA. The siRNA may include one or more internucleoside linkages and/or one or more nucleoside modifications. Any of the aforementioned siRNAs may include a sense strand that lacks a 3′ A of a sense strand sequence in Table 5. Any of the aforementioned siRNAs may include a sense strand that lacks a 5′ U of an antisense strand sequence in Table 5.

In some embodiments, the siRNA comprises the sense strand and/or the antisense strand sequence of an siRNA of Table 14, or a nucleic acid sequence thereof having 3 or 4 nucleoside substitutions, additions, or deletions. In some embodiments, the siRNA comprises the sense strand and/or the antisense strand sequence of an siRNA of Table 14, or a nucleic acid sequence thereof having 1 or 2 nucleoside substitutions, additions, or deletions. In some embodiments, the siRNA comprises the sense strand and/or the antisense strand sequence of an siRNA of Table 14. In some embodiments, the siRNA is cross-reactive with a non-human primate (NHP) HGFAC mRNA. The siRNA may include one or more internucleoside linkages and/or one or more nucleoside modifications. Any of the aforementioned siRNAs may include a sense strand that lacks a 3′ A of a sense strand sequence in Table 14. Any of the aforementioned siRNAs may include a sense strand that lacks a 5′ U of an antisense strand sequence in Table 14.

### B. ASOs

In some embodiments, the composition comprises an oligonucleotide that inhibits the expression of HGFAC, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of HGFAC, wherein the oligonucleotide comprises an ASO about 12-30 nucleosides in length and comprising a nucleoside sequence complementary to about 12-30 contiguous nucleosides of a full-length human HGFAC mRNA sequence such as SEQ ID NO: 4803; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 4803.

### C. Modifications

In some embodiments, the composition comprises an oligonucleotide that inhibits the expression of HGFAC, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. A phosphorothioate may include a nonbridging oxygen atom in a phosphate backbone of the oligonucleotide that is replaced by sulfur. Modified internucleoside linkages may be included in siRNAs or ASOs. Benefits of the modified internucleoside linkage may include decreased toxicity or improved pharmacokinetics.

In some embodiments, the composition comprises an oligonucleotide that inhibits the expression of HGFAC, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of HGFAC, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that inhibits the expression of HGFAC, wherein the oligonucleotide comprises a moiety attached at a 3′ or 5′ terminus of the oligonucleotide. Examples of moieties include a hydrophobic moiety or a sugar moiety, or a combination thereof. In some embodiments, the oligonucleotide is an siRNA having a sense strand, and the moiety is attached to a 5′ end of the sense strand. In some embodiments, the oligonucleotide is an siRNA having a sense strand, and the moiety is attached to a 3′ end of the sense strand. In some embodiments, the oligonucleotide is an siRNA having an antisense strand, and the moiety is attached to a 5′ end of the antisense strand. In some embodiments, the oligonucleotide is an siRNA having an antisense strand, and the moiety is attached to a 3′ end of the antisense strand. In some embodiments, the oligonucleotide is an ASO, and the moiety is attached to a 5′ end of the ASO. In some embodiments, the oligonucleotide is an ASO, and the moiety is attached to a 3′ end of the ASO.

In some embodiments, the composition comprises an oligonucleotide that inhibits the expression of HGFAC, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of HGFAC, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the composition comprises an oligonucleotide that inhibits the expression of HGFAC, wherein the oligonucleotide comprises a sugar moiety. The sugar moiety may include an N-acetyl galactose moiety (e.g., an N-acetylgalactosamine (GalNAc) moiety), an N-acetyl glucose moiety (e.g., an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages since they may target or bind a mannose receptor such as CD206.

In some embodiments, the composition comprises an oligonucleotide that inhibits the expression of HGFAC, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′ fluoro modified purines. In some embodiments, purines of the oligonucleotide comprise 2′-O-methyl modified purines. In some embodiments, purines of the oligonucleotide comprise a mixture of 2′ fluoro and 2′-O-methyl modified purines. In some embodiments, all purines of the oligonucleotide comprise 2′ fluoro modified purines. In some embodiments, all purines of the oligonucleotide comprise 2′-O-methyl modified purines. In some embodiments, all purines of the oligonucleotide comprise a mixture of 2′ fluoro and 2′-O-methyl modified purines. In some embodiments, 2′-O-methyl includes 2′ O-methyl.

In some embodiments, pyrimidines of the oligonucleotide comprise 2′ fluoro modified pyrimidines. In some embodiments, pyrimidines of the oligonucleotide comprise 2′-O-methyl modified pyrimidines. In some embodiments, pyrimidines of the oligonucleotide comprise a mixture of 2′ fluoro and 2′-O-methyl modified pyrimidines. In some embodiments, all pyrimidines of the oligonucleotide comprise 2′ fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise a mixture of 2′ fluoro and 2′-O-methyl modified pyrimidines. In some embodiments, purines of the oligonucleotide comprise 2′-O-methyl modified purines, and pyrimidines of the oligonucleotide comprise a mixture of 2′ fluoro and 2′-O-methyl modified pyrimidines. In some embodiments, purines of the oligonucleotide comprise 2′ fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′ fluoro modified pyrimidines. In some embodiments, pyrimidines of the oligonucleotide comprise 2′ fluoro modified pyrimidines, and purines of the oligonucleotide comprise a mixture of 2′ fluoro and 2′-O-methyl modified purines. In some embodiments, pyrimidines of the oligonucleotide comprise 2′-O-methyl modified pyrimidines, and purines of the oligonucleotide comprise a mixture of 2′ fluoro and 2′-O-methyl modified purines. In some embodiments, pyrimidines of the oligonucleotide comprise 2′ fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′ fluoro modified purines.

In some embodiments, all purines of the oligonucleotide comprise 2′ fluoro modified purines, and all pyrimidines of the oligonucleotide comprise a mixture of 2′ fluoro and 2′-O-methyl modified pyrimidines. In some embodiments, all purines of the oligonucleotide comprise 2′-O-methyl modified purines, and all pyrimidines of the oligonucleotide comprise a mixture of 2′ fluoro and 2′-O-methyl modified pyrimidines. In some embodiments, all purines of the oligonucleotide comprise 2′ fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′ fluoro modified pyrimidines. In some embodiments, all pyrimidines of the oligonucleotide comprise 2′ fluoro modified pyrimidines, and all purines of the oligonucleotide comprise a mixture of 2′ fluoro and 2′-O-methyl modified purines. In some embodiments, all pyrimidines of the oligonucleotide comprise 2′-O-methyl modified pyrimidines, and all purines of the oligonucleotide comprise a mixture of 2′ fluoro and 2′-O-methyl modified purines. In some embodiments, all pyrimidines of the oligonucleotide comprise 2′ fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′ fluoro modified purines.

In some cases, the oligonucleotide comprises a particular modification pattern. In some embodiments, position 9 counting from the 5′ end of the of a strand of the oligonucleotide may have a 2′F modification. In some embodiments, when position 9 of a strand of the oligonucleotide is a pyrimidine, then all purines in a strand of the oligonucleotide have a 2′OMe modification. In some embodiments, when position 9 is the only pyrimidine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are pyrimidines, then both of these pyrimidines are the only two positions with a 2′F modification in a strand of the oligonucleotide. In some embodiments, when position 9 and only two other bases between positions 5 and 11 of a strand of the oligonucleotide are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of a strand of the oligonucleotide, then all combinations of pyrimidines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some embodiments, when position 9 of a strand of the oligonucleotide is a purine, then all purines in a strand of the oligonucleotide have a 2′OMe modification. In some embodiments, when position 9 is the only purine between positions 5 and 11 of the sense stand, then position 9 is the only position with a 2′F modification in a strand of the oligonucleotide. In some embodiments, when position 9 and only one other base between positions 5 and 11 of a strand of the oligonucleotide are purines, then both of these purines are the only two positions with a 2′F modification in a strand of the oligonucleotide. In some embodiments, when position 9 and only two other bases between positions 5 and 11 of a strand of the oligonucleotide are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of a strand of the oligonucleotide, then all combinations of purines having the 2′F modification are allowed that have three to five 2′F modifications in total, provided that a strand of the oligonucleotide does not have three 2′F modifications in a row. In some cases, a strand of the oligonucleotide of any of the siRNAs comprises a modification pattern which conforms to any or all of these a strand of the oligonucleotide rules.

In some cases, position 9 of a strand of the oligonucleotide can be a 2′deoxy. In these cases, 2′F and 2′OMe modifications may occur at the other positions of a strand of the oligonucleotide. In some cases, a strand of the oligonucleotide of any of the siRNAs comprises a modification pattern which conforms to these a strand of the oligonucleotide rules.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′flouro-modified pyrimidine, provided there are not three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′flouro-modified pyrimidine, provided there are not three 2′ fluoro-modified pyrimidines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′flouro-modified purine, provided there are not three 2′ fluoro-modified purine in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, the even-numbered positions of the antisense strand comprise 2′flouro-modified nucleotides, 2′-O-methyl modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′flouro-modified purines, provided there are not three 2′ fluoro-modified purines in a row; the odd-numbered positions of the antisense strand comprise 2′-O-methyl modified nucleotides; and the even-numbered positions of the antisense strand comprise 2′flouro-modified nucleotides and unmodified deoxyribonucleotides. In some embodiments, there are not three 2′ fluoro-modified purines in a row. In some embodiments, there are not three 2′ fluoro-modified pyrimidines in a row.

In some embodiments, position nine of the sense strand comprises an unmodified deoxyribonucleotide. In some embodiments, positions 5, 7, and 8 of the sense strand comprise 2′fluoro-modified nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, the even-numbered positions of the antisense strand comprise 2′flouro-modified nucleotides, 2′-O-methyl modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′flouro-modified nucleotides and unmodified deoxyribonucleotide.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

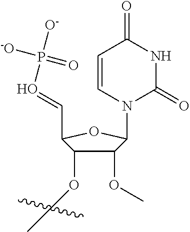

### 5′ vinylphosphonate 2′ O Methyl Uridine

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### 1. Hydrophobic Moieties

In some embodiments, the composition comprises an oligonucleotide that inhibits the expression of HGFAC, wherein the oligonucleotide comprises a hydrophobic moiety. The hydrophobic moiety may be attached at a 3′ or 5′ terminus of the oligonucleotide. The hydrophobic moiety may include a lipid such as a fatty acid. The hydrophobic moiety may include a hydrocarbon. The hydrocarbon may be linear. The hydrocarbon may be non-linear. The hydrophobic moiety may include a lipid moiety or a cholesterol moiety, or a combination thereof.

In some embodiments, the composition comprises an oligonucleotide that inhibits the expression of HGFAC, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof.

In some embodiments, the composition comprises an oligonucleotide that inhibits the expression of HGFAC, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of HGFAC, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl, stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

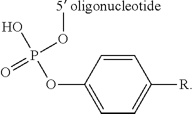

In some embodiments, the oligonucleotide comprises any aspect of the following structure:

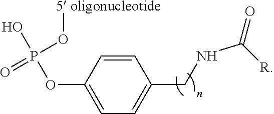

In some embodiments, the oligonucleotide comprises any aspect of the following structure:

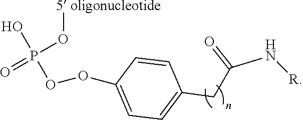

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1B. The example lipid moieties in Table 1B are shown attached at a 5′ end of an oligonucleotide, in which the 5′ terminal phosphate of the oligonucleotide is shown with the lipid moiety. In some embodiments, a lipid moiety in Table 1B may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1B
Hydrophobic moiety examples

| Hydrophobic | Hydrophobic | |
|---|---|---|
| Moiety Description | Moiety Name | Example Conjugation |
| stearyl | ETL3 | 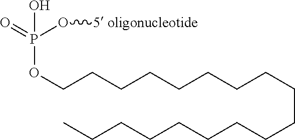 |
| t-butylphenyl | ETL7 | 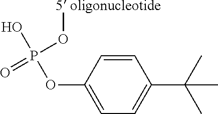 |
| n-butylphenyl | ETL8 | 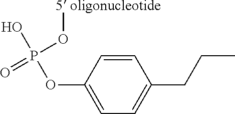 |
| octylphenyl | ETL9 | 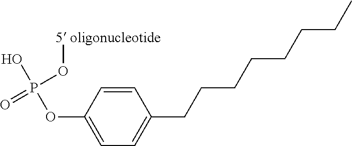 |
| dodecylphenyl | ETL10 | 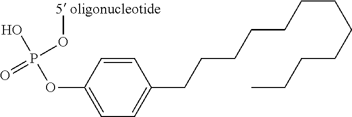 |
| phenyl n-dodecyl | ETL12 | 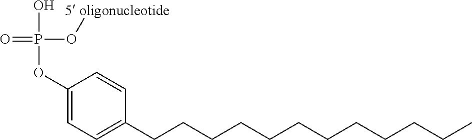 |
| octadecylbenzamide | ETL13 | 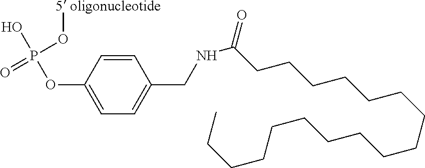 |
| hexadecylbenzamide | ETL15 | 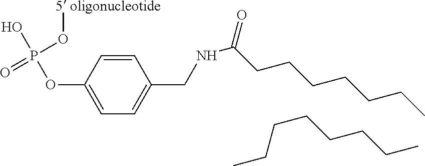 |
| octadecylcyclohexyl | ETL16 | 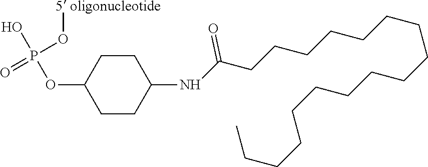 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the para, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure

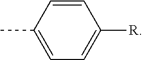

In some embodiments, the lipid moiety comprises or consists of the following structure:

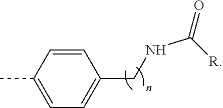

In some embodiments, the lipid moiety comprises the following structure:

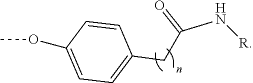

In some embodiments, the lipid moiety comprises or consist of the following structure:

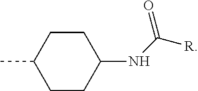

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

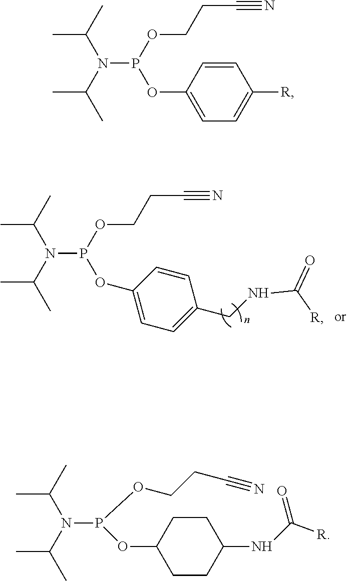

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

The modifications described herein may be useful for delivery to a cell or tissue, for example, extrahepatic delivery or targeting of an oligonucleotide composition. The modifications described herein may be useful for targeting an oligonucleotide composition to a cell or tissue.

### 2. Sugar Moieties

In some embodiments, the composition comprises an oligonucleotide that inhibits the expression of HGFAC, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of HGFAC, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker.

The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of HGFAC, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

Disclosed herein, in some embodiments, are compositions comprising an oligonucleotide that inhibits the expression of HGFAC, wherein the oligonucleotide comprises a GalNAc moiety. The GalNAc moiety may be included in any formula, structure, or GalNAc moiety shown below. In some embodiments, described herein is a compound (e.g. oligonucleotide) represented by Formula (I) or (II):

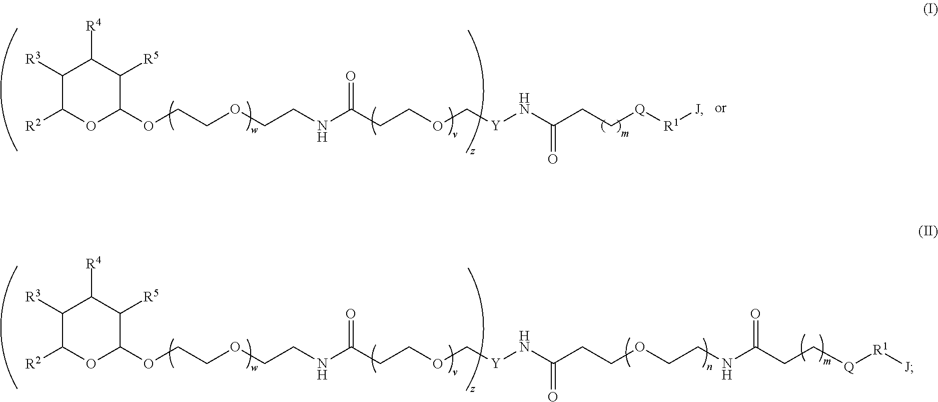

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O—)O—, —SP(O)(O—)O—, —OP(S)(O—)O—, —OP(O)(S—)O—, —OP(O)(O—)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
   C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
   —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁷ is independently selected from:
   hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.
      In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O—)O—, —SP(O)(O—)O—, —OP(S)(O—)O—, —OP(O)(S—)O—, —OP(O)(O—)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂—NR⁷. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O—)O—, —SP(O)(O—)O—, —OP(S)(O—)O—, —OP(O)(S—)O—, —OP(O)(0-)S—, and —OP(OR⁷)O—. In some embodiments, R¹ is selected from —OP(O)(OR⁷)O—, —OP(S)(OR⁷)O—, —OP(O)(O—)O—, —OP(S)(O—)O—, —OP(O)(S—)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷—SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷—SR⁷, —OC(O)R⁷, and —N(R⁷)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR⁷)O—, —OP(S)(OR⁷)O—, —OP(O)(O—)O—, —OP(S)(O—)O—, —OP(O)(S—)O—, and —OP(OR⁷)O—; R² is C₁ alkyl substituted with —OH or —OC(O)CH₃;
R³ is —OH or —OC(O)CH₃; R⁴ is —OH or —OC(O)CH₃; and R⁵ is —NH(O)CH₃. In some embodiments, the compound comprises:

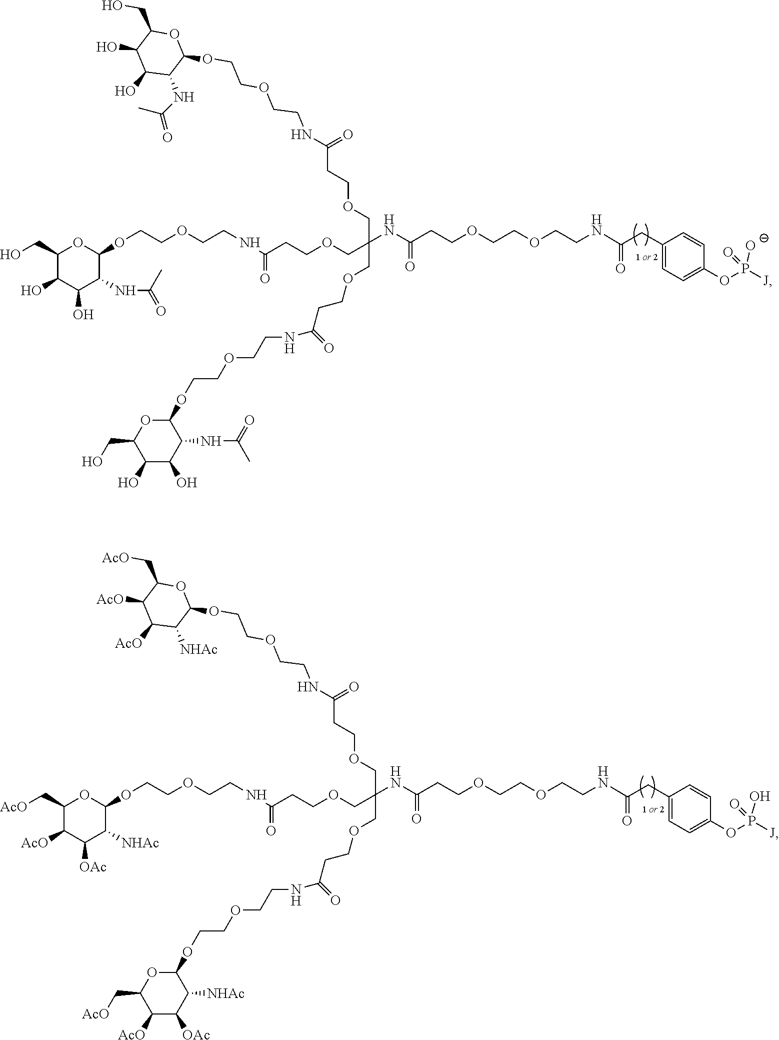

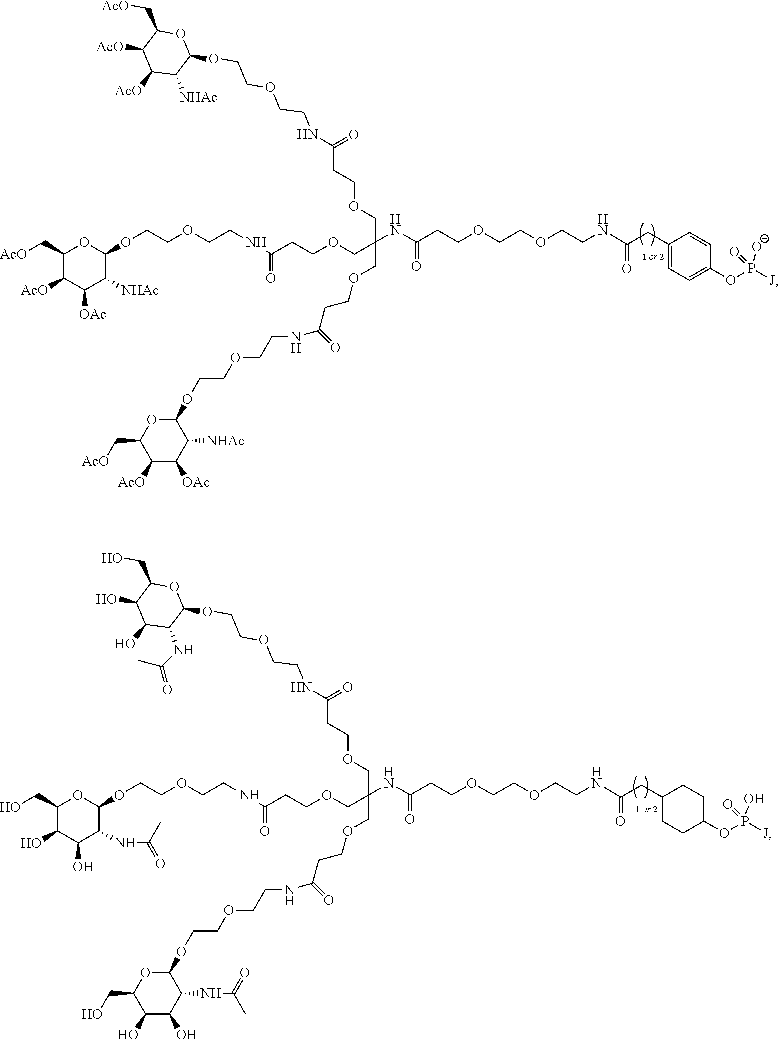

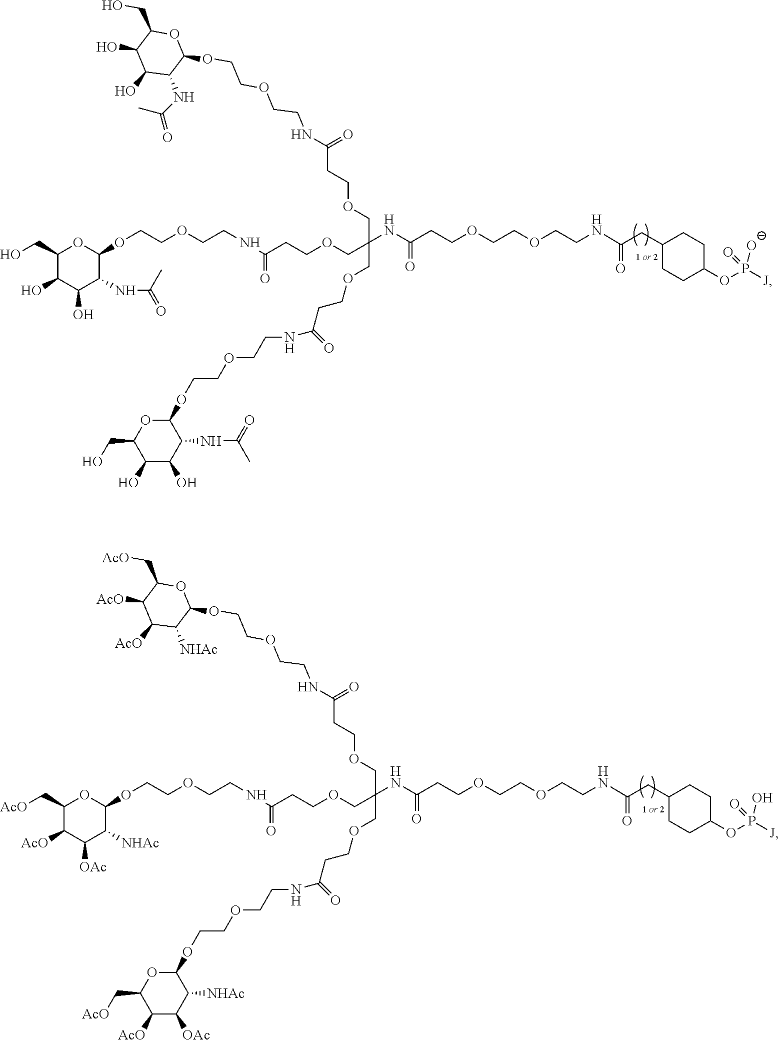

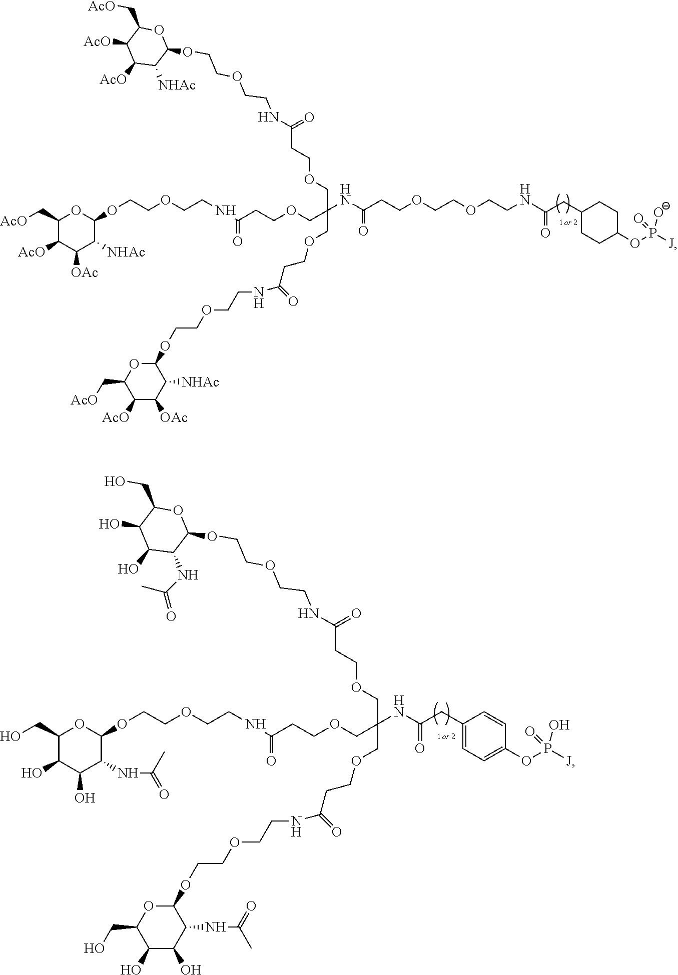

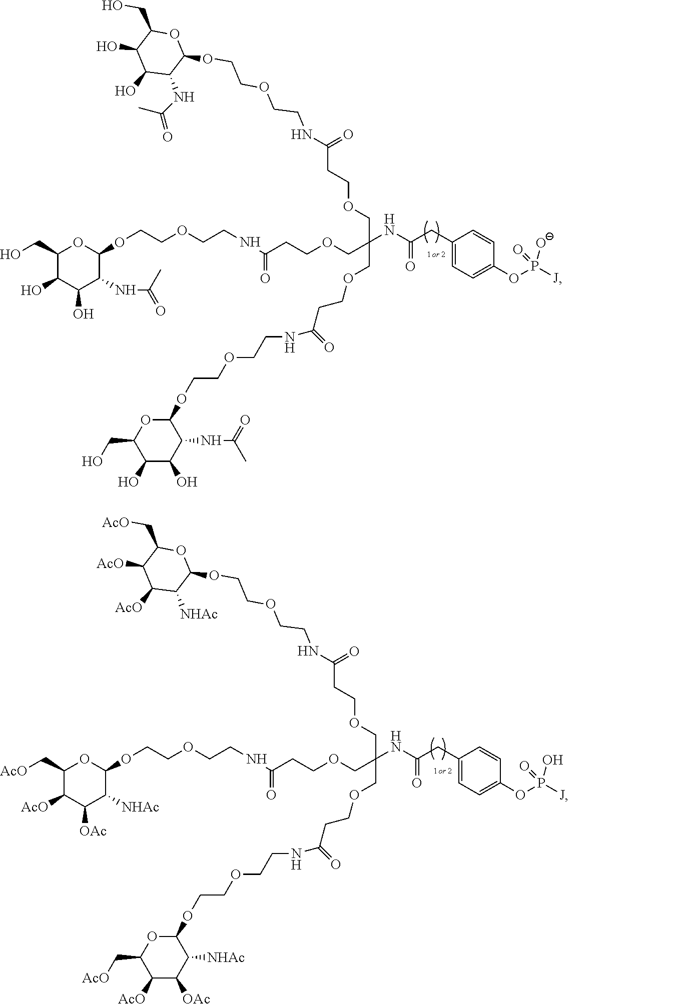

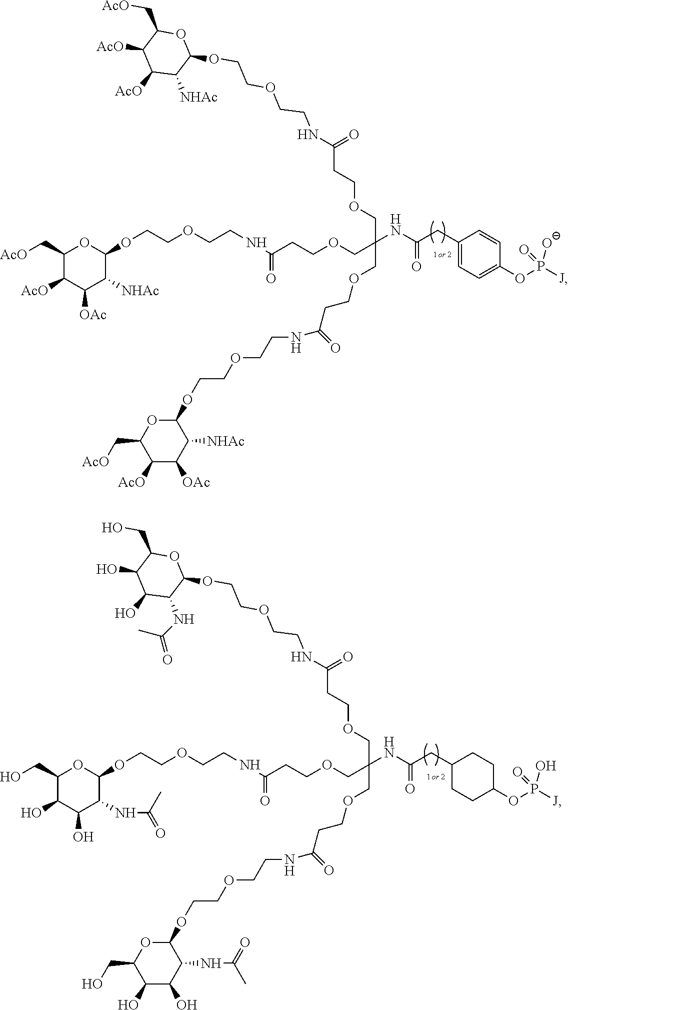

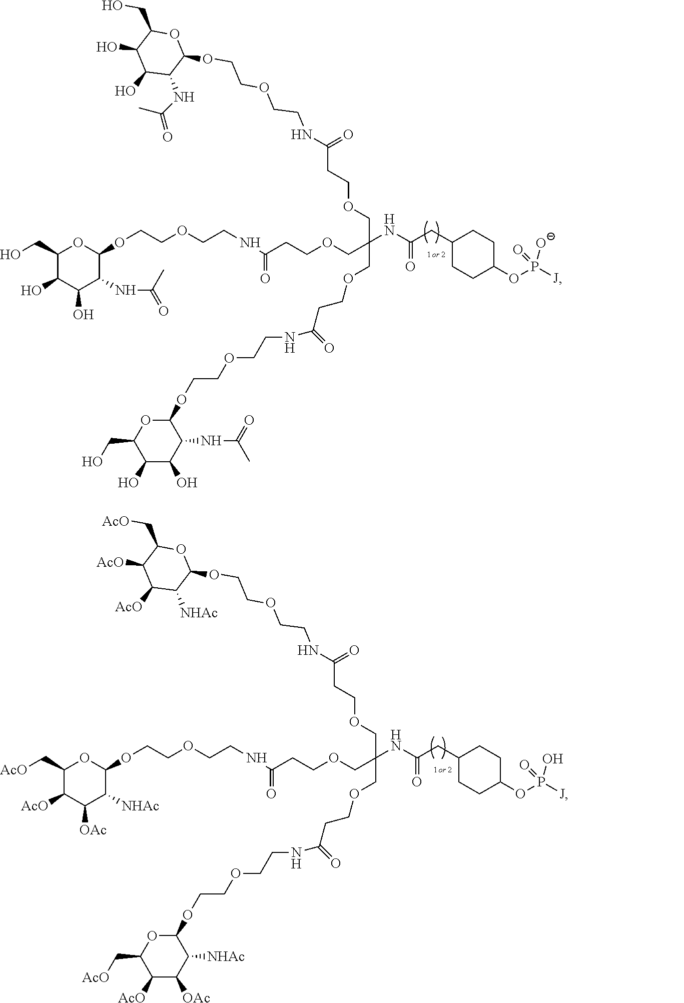

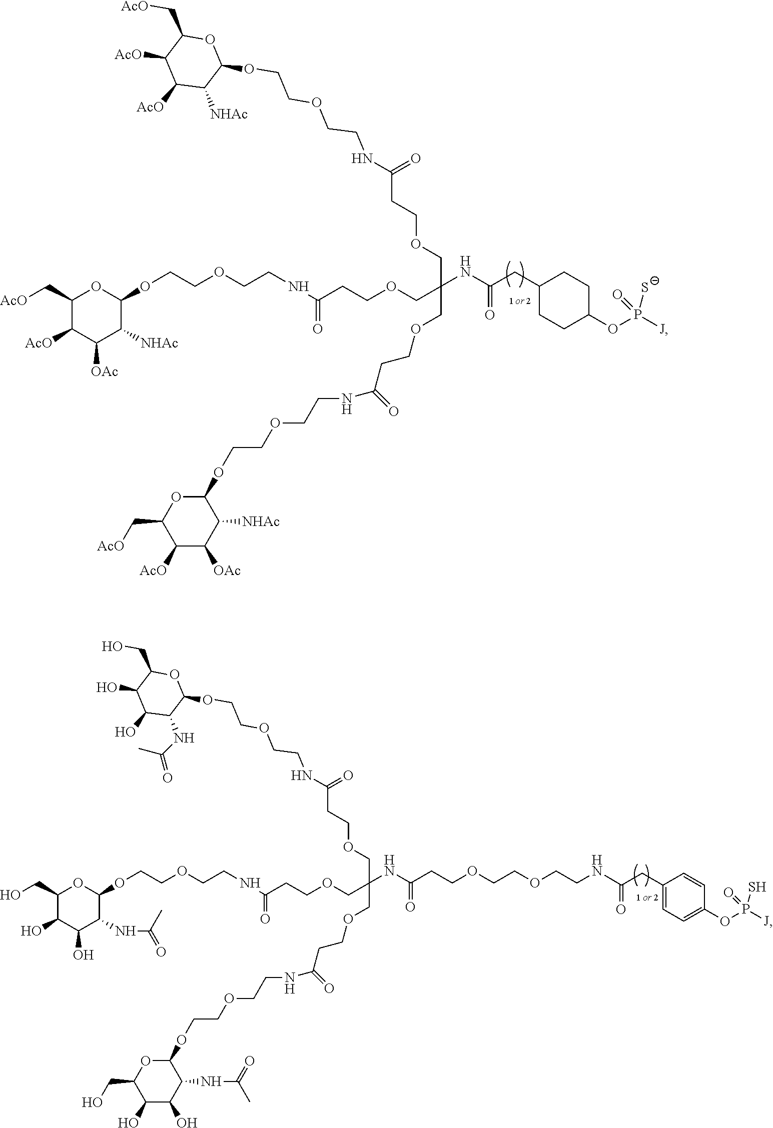

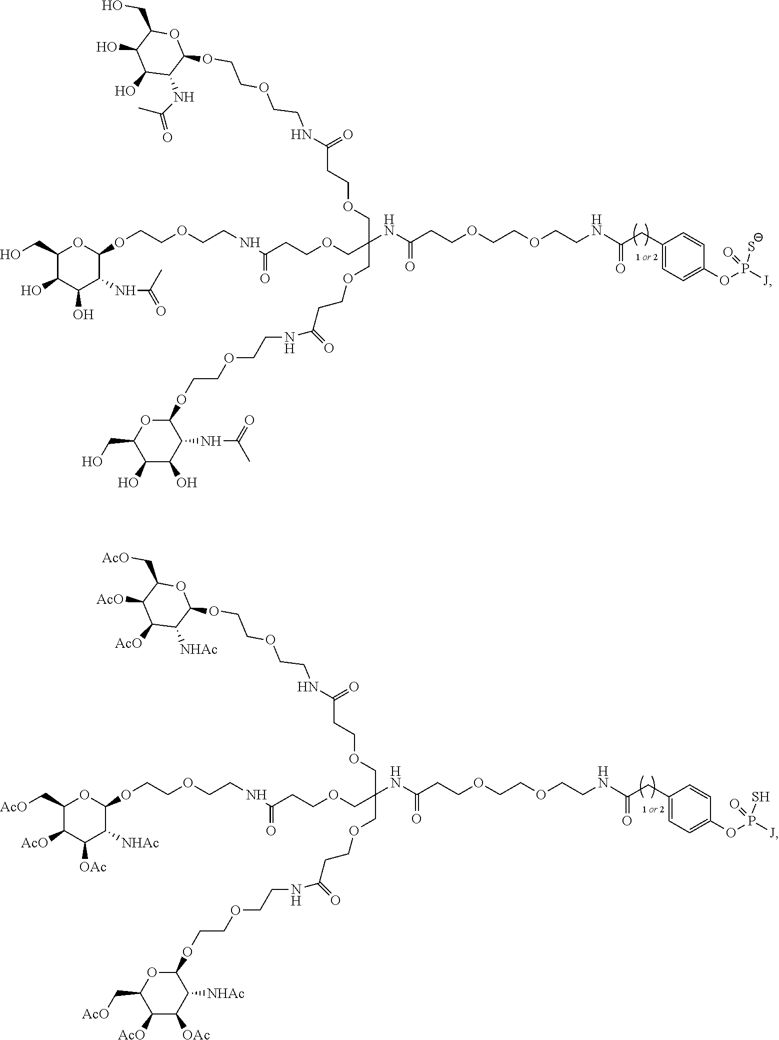

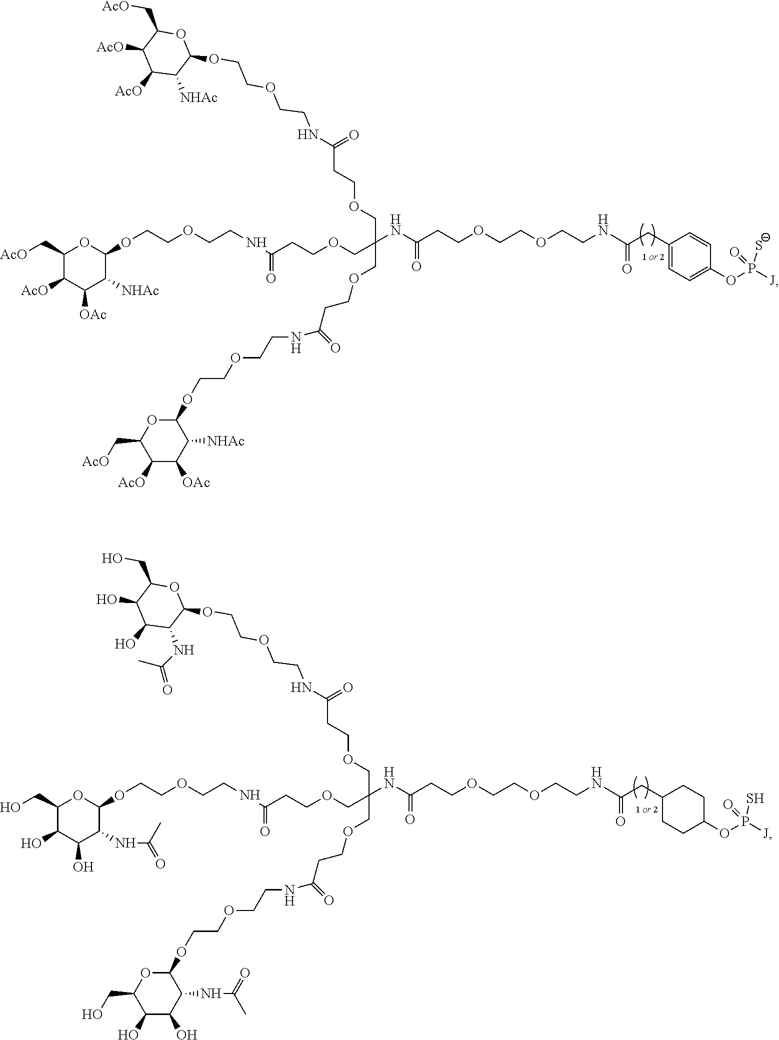

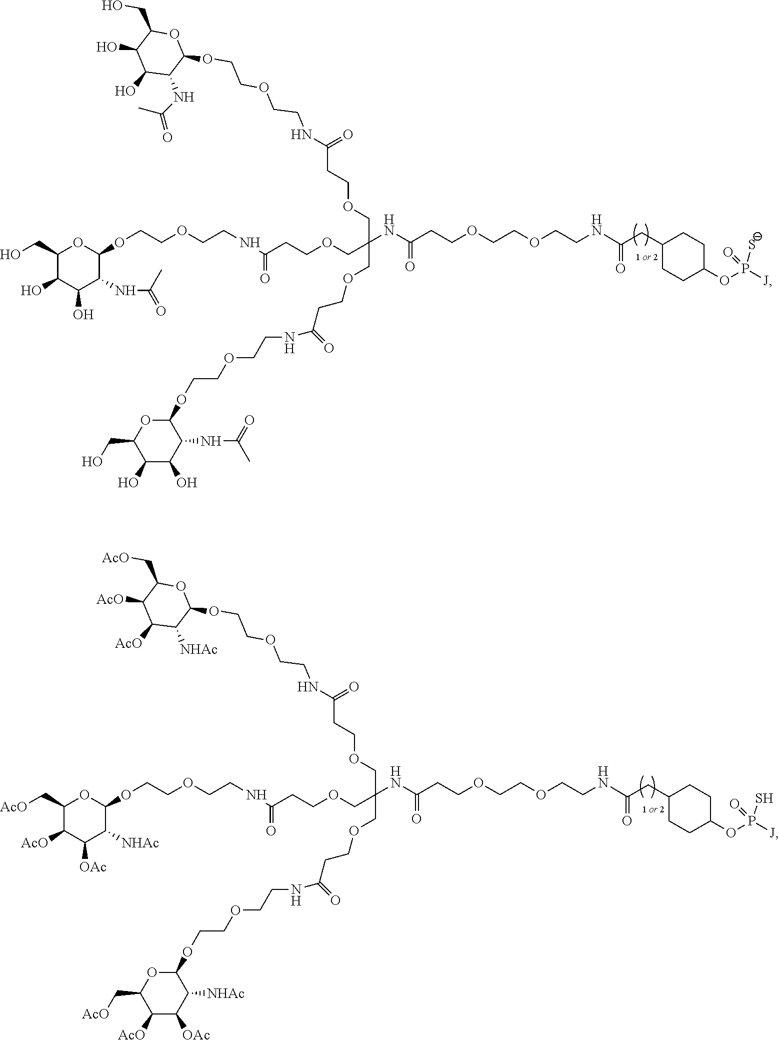

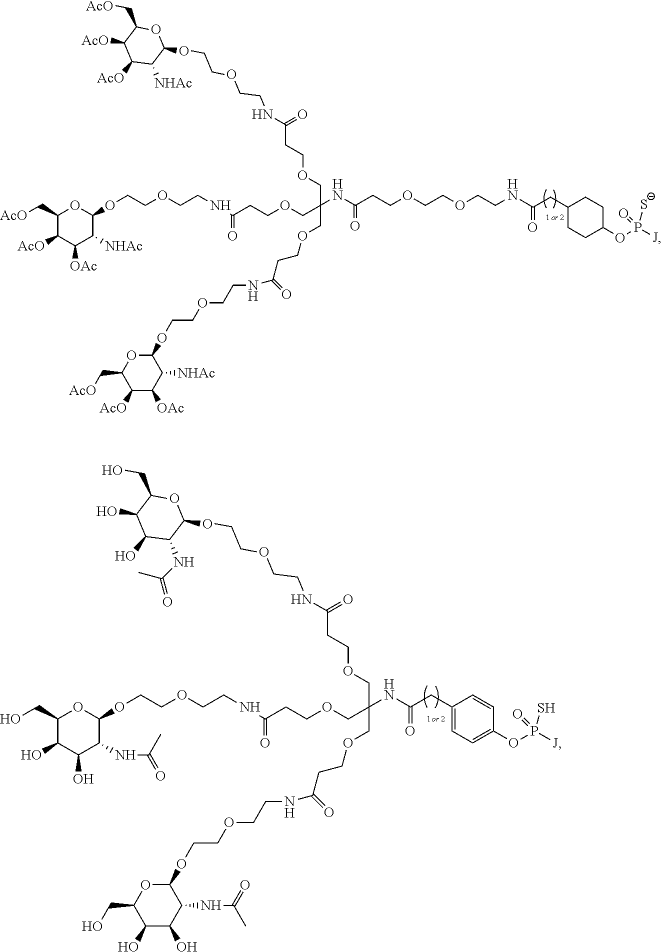

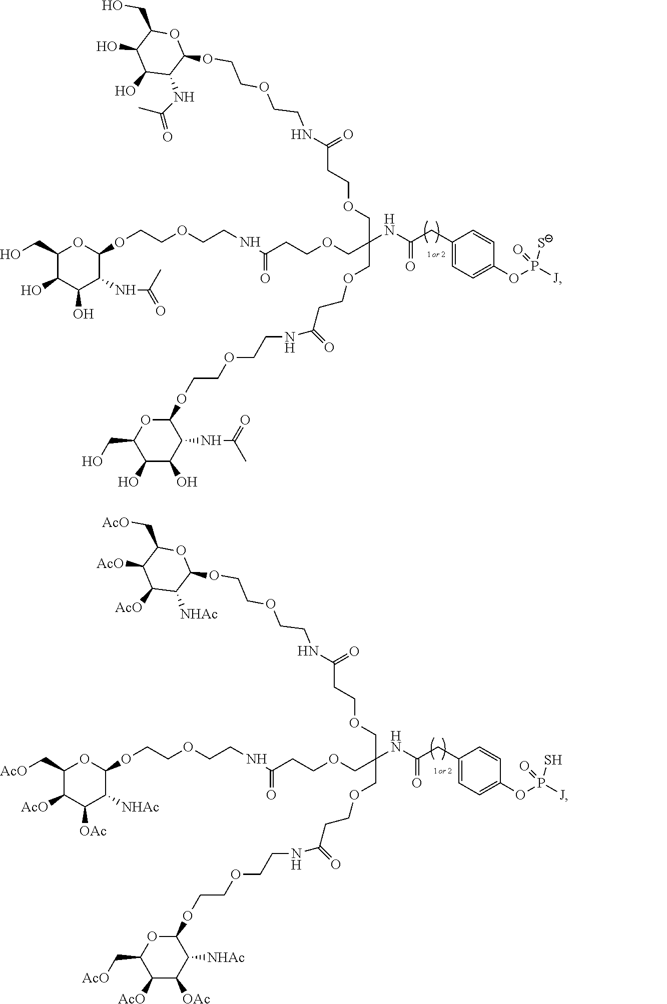

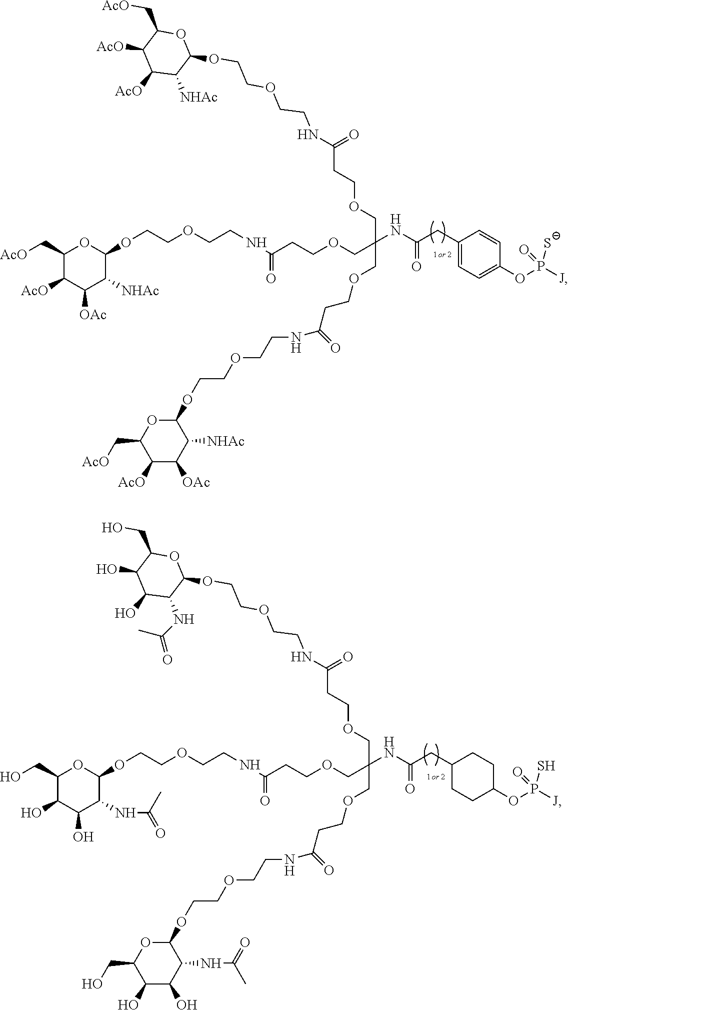

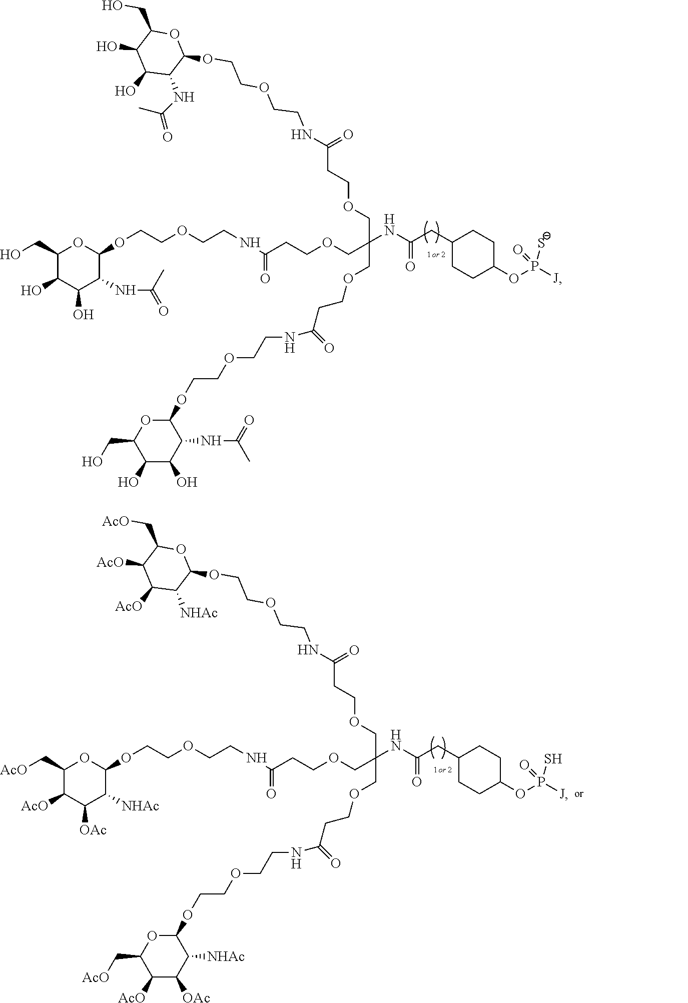

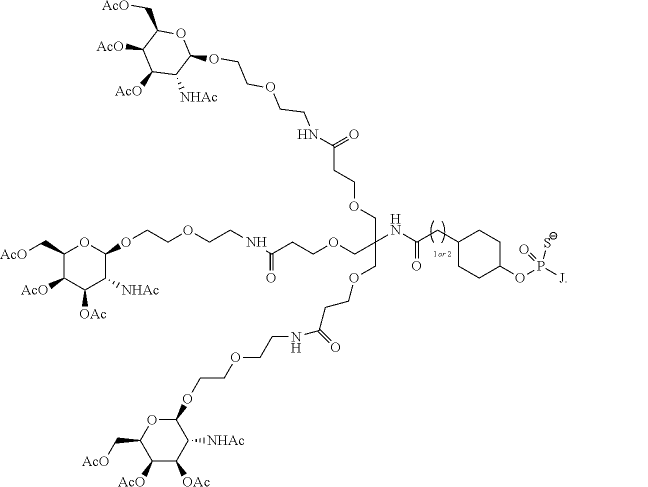

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

Some embodiments include the following, where J is the oligonucleotide:

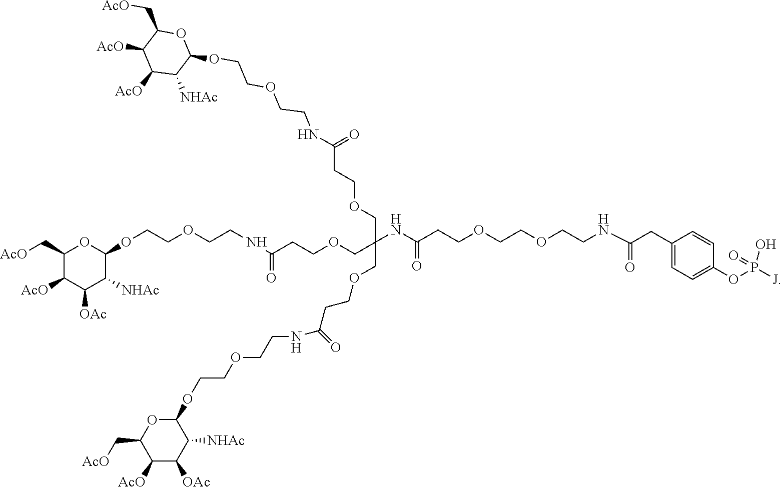

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

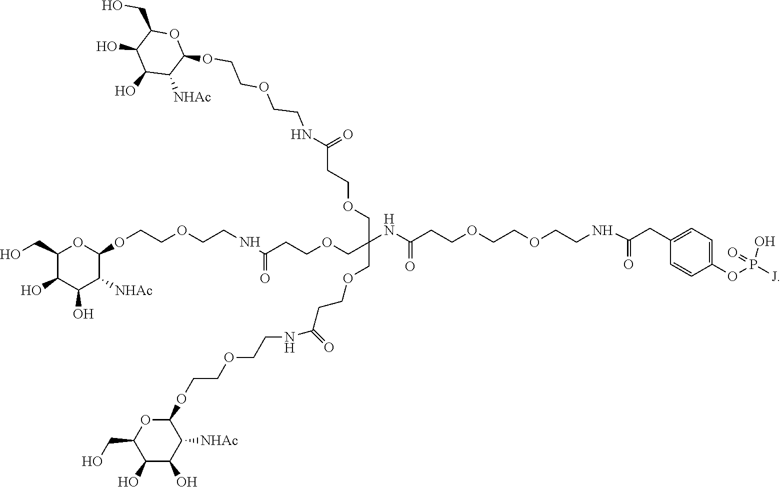

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

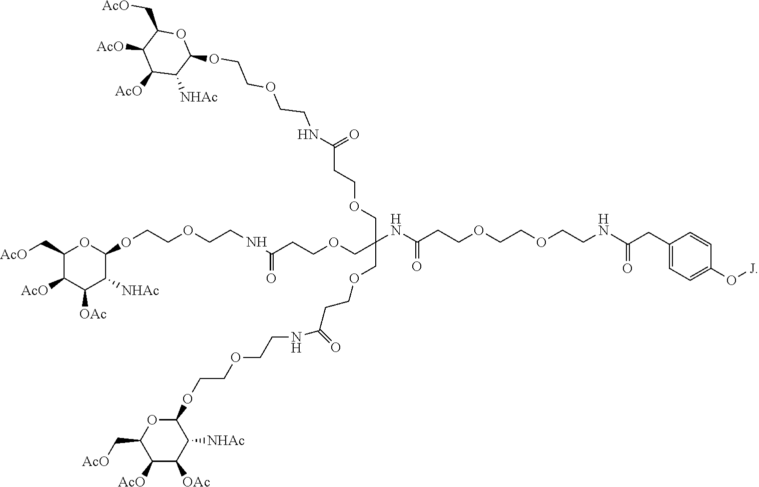

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

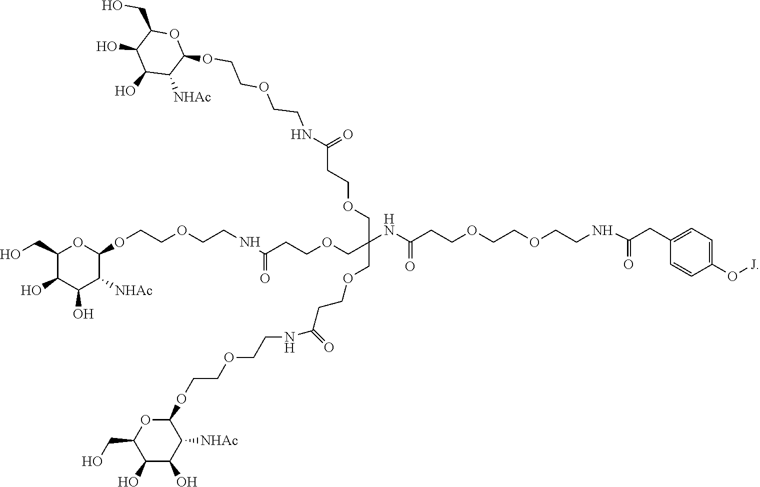

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where the phosphate or “5′” indicates a connection to the oligonucleotide:

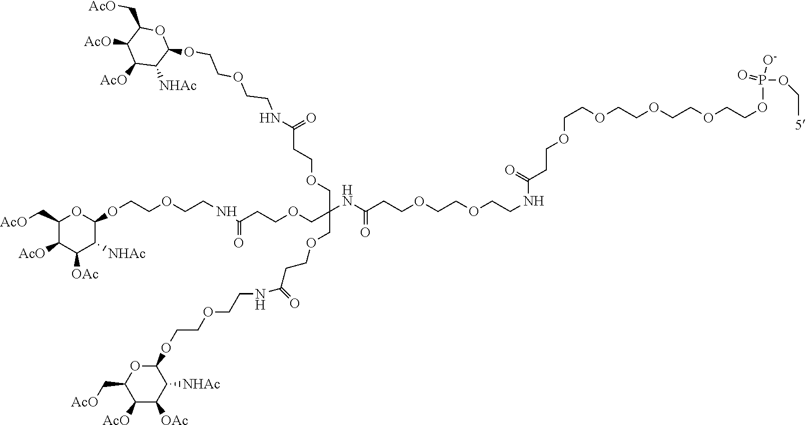 [SMILES: CCOP(=O)([O-])OCCOCCOCCOCCOCCC(=O)NCCOCCOCCC(=O)NC(COCCC(=O)NCCOCCO[C@@H]1O[C@H](COC(C)=O)[C@H](OC(C)=O)[C@H](OC(C)=O)[C@H]1NC(C)=O)(COCCC(=O)NCCOCCO[C@@H]1O[C@H](COC(C)=O)[C@H](OC(C)=O)[C@H](OC(C)=O)[C@H]1NC(C)=O)COCCC(=O)NCCOCCO[C@@H]1O[C@H](COC(C)=O)[C@H](OC(C)=O)[C@H](OC(C)=O)[C@H]1NC(C)=O]

Some embodiments include the following, where the phosphate or “5′” indicates a connection to the oligonucleotide:

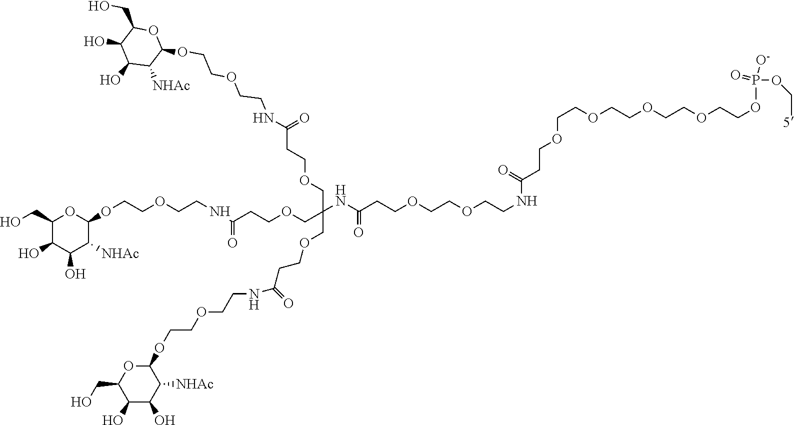 [SMILES: CCOP(=O)([O-])OCCOCCOCCOCCOCCC(=O)NCCOCCOCCC(=O)NC(COCCC(=O)NCCOCCO[C@@H]1O[C@H](CO)[C@H](O)[C@H](O)[C@H]1NC(C)=O)(COCCC(=O)NCCOCCO[C@@H]1O[C@H](CO)[C@H](O)[C@H](O)[C@H]1NC(C)=O)COCCC(=O)NCCOCCO[C@@H]1O[C@H](CO)[C@H](O)[C@H](O)[C@H]1NC(C)=O]

Some embodiments include the following, where J is the oligonucleotide:

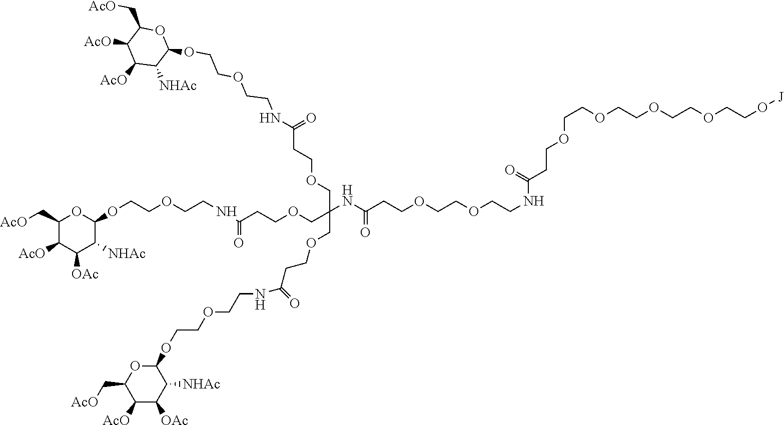

include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide.

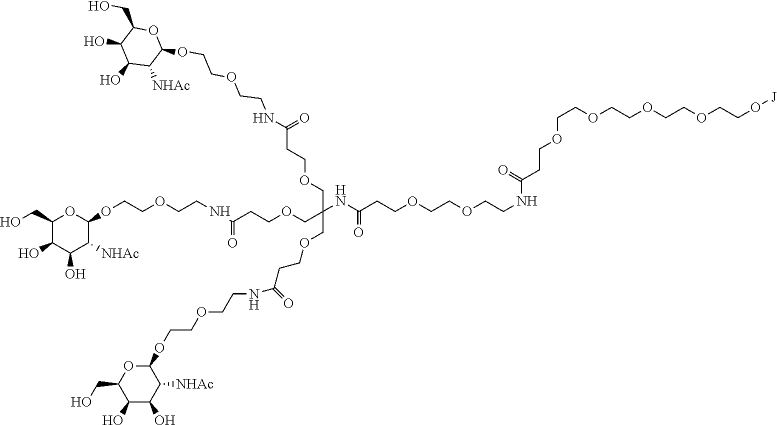

The structure in this compound attached to the oligonucleotide (J) may be referred to as “ETL1,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide

Some embodiments include the following, where J is the oligonucleotide:

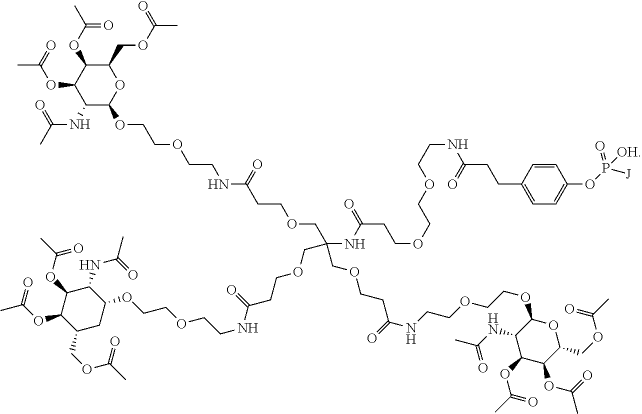

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

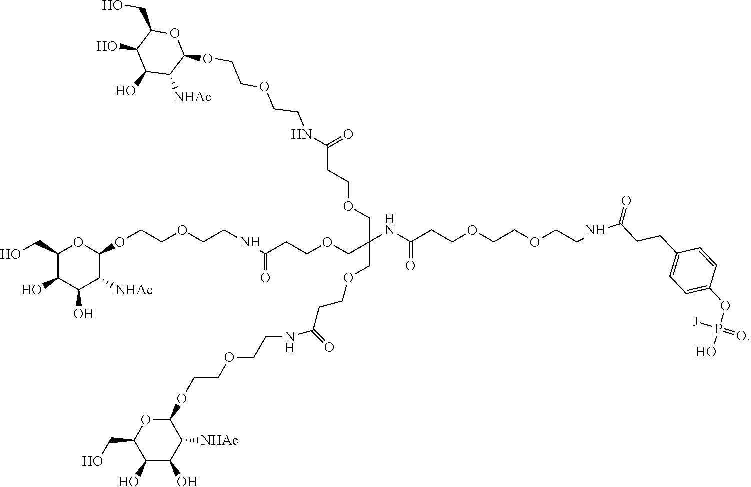

J may include one or more additional phosphates, or one or more phosphorothioates linking to the oligonucleotide. J may include one or more additional phosphates linking to the oligonucleotide. J may include one or more phosphorothioates linking to the oligonucleotide.

Some embodiments include the following, where J is the oligonucleotide:

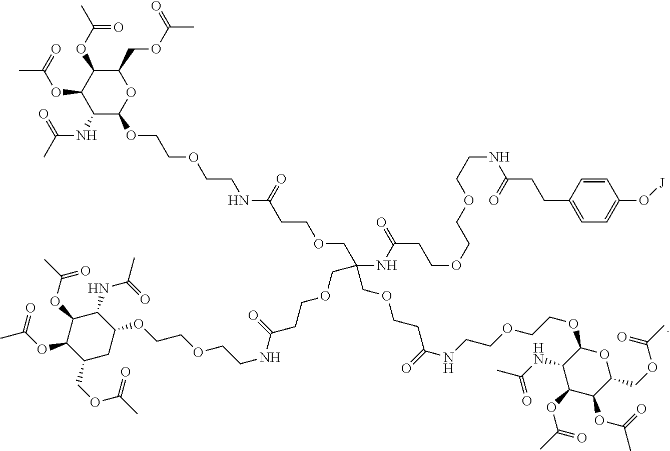

J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.

Some embodiments include the following, where J is the oligonucleotide:

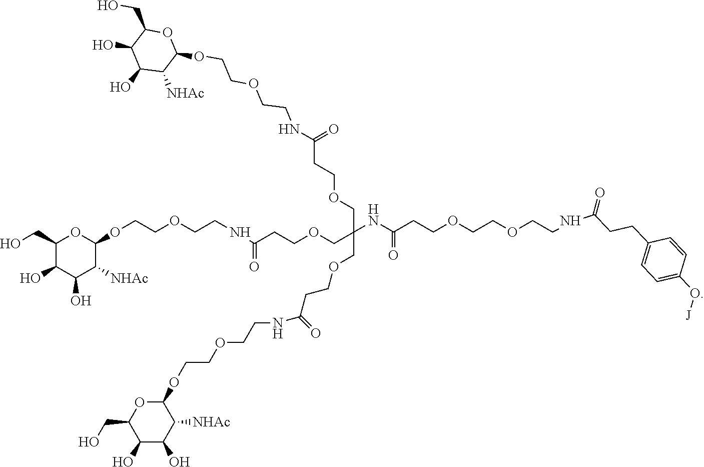

The structure in this compound attached to the oligonucleotide (J) may be referred to as “ETL17,” and is an example of a GalNAc moiety. J may include one or more phosphates or phosphorothioates linking to the oligonucleotide. J may include one or more phosphates linking to the oligonucleotide. J may include a phosphate linking to the oligonucleotide. J may include one or more phosphorothioates linking to the oligonucleotide. J may include a phosphorothioate linking to the oligonucleotide.
3. Modified siRNAs

In some embodiments, the composition comprises an oligonucleotide that inhibits the expression of HGFAC, wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-moiety-3′, wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-moiety-3′, wherein “Nf” is a 2′ fluoro-modified nucleoside, “n” is a 2′ O-methyl modified nucleoside, “s” is a phosphorothioate linkage, and N comprises one or more nucleosides. In some embodiments, the moiety in modification pattern 4S or 5S is a sugar moiety. In some embodiments, the sense strand comprises modification pattern 6S: 5′-NfsnsNfnNfnNfnNfnNfnNfnNfnNfnNfsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 7S: 5′-nsnsnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nsnsnnnNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nsnsnnnnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 10S: 5′-nnnnnNfNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 11S: 5′-nnnnnnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 12S: 5′-nnnnNfnNfNfdNNfnnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 13S: 5′-nnnnnnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 14S: 5′-nnnnnNfnnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 15S: 5′-nnnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 16S: 5′-nnnnnNfnNfNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 17S: 5′-nnnnnnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 18S: 5′-nnnnNfnNfnNfhnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 19S: 5′-nnnnNfNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 20S: 5′-nnnnNfnnnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 21S: 5′-nnnnNfNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 22S: 5′-nnnnnnNfnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 23S: 5′-nnnnnNfNfnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 24S: 5′-nnnnnNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 25S: 5′-nnnnnNfNfNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 26S: 5′-nnnnNfnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 27S: 5′-nnnnNfnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 28S: 5′-nnnnNfnNfnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 29S: 5′-nnnnNfNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 30S: 5′-nnnnNfNfnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 31S: 5′-nnnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 32S: 5′-nnnnnnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 33S: 5′-nnnnnnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 34S: 5′-nnnnnnNfNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 35S: 5′-nnnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 36S: 5′-nnnnnNfnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 37S: 5′-nnnnnNfNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 38S: 5′-nnnnnNfNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 39S: 5′-nnnnNfnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 40S: 5′-nnnnNfnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 41S: 5′-nnnnNfnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 42S: 5′-nnnnNfnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 43S: 5′-nnnnNfNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 44S: 5′-nnnnnnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 45S: 5′-nnnnNfnNfNfdNnnnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 46S: 5′-nnnnnnnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 47S: 5′-nnnnNfnNfNfdTnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 48S: 5′-nnnnNfnNfNfdNnNfnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 49S: 5′-nnnnNfnNfNfdTnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 50S: 5′-snnnnnNfNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 51S: 5′-snnnnNfnNfNfdNNfnnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 52S: 5′-snnnnnNfNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 53S: 5′-snnnnNfnNfNfdNNfnnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 54S: 5′-snnnnnNfnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 55S: 5′-snnnnnnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 56S: 5′-snnnnNfNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 57S: 5′-snnnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 58S: 5′-snnnnNfNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 59S: 5′-snnnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 60S: 5′-snnnnNfnNfNfdNnnnnnnnnnnsnsn-3′, wherein “Nf” is a 2′ fluoro-modified nucleoside, “n” is a 2′ O-methyl modified nucleoside, “dN” is a 2′ deoxy nucleoside, and “s” is a phosphorothioate linkage. In some embodiments, the sense strand comprises modification pattern 61S: 5′-snnnnNfNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 62S: 5′-snnnnNfnnNfNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 63S: 5′-snnnnNfnNfNfdTnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 64S: 5′-snnnnNfnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 65S: 5′-snnnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 66S: 5′-snnnnNfnNfNfdNnNfnnnnnnnnsnsn-3′, “dN” is a 2′ deoxy nucleoside, wherein “Nf” is a 2′ fluoro-modified nucleoside, “n” is a 2′ O-methyl modified nucleoside, and “s” is a phosphorothioate linkage. In some embodiments, the sense strand comprises modification pattern 67S: 5′-snnnnNfnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 68S: 5′-snnnnnNfNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 69S: 5′-snnnnNfnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 70S: 5′-snnnnnnNfnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 71S: 5′-snnnnNfnNfnNfnnnnnnnnnnsnsn-3′, wherein “Nf” is a 2′ fluoro-modified nucleoside, “n” is a 2′ O-methyl modified nucleoside, “m” is a methyoxyethyl modified nucleoside, and “s” is a phosphorothioate linkage. In some embodiments, the sense strand comprises modification pattern 72S: 5′-snnnnNfnnnNfNfnnnnnnnnnsnsn-3′, wherein “Nf” is a 2′ fluoro-modified nucleoside, “n” is a 2′ O-methyl modified nucleoside, “m” is a methyoxyethyl modified nucleoside, and “s” is a phosphorothioate linkage. In some embodiments, the sense strand comprises modification pattern 73S: 5′-snnnNmnNfNfNfNfnnnmnnnnnnsnsn-3′, wherein “Nm” is a 2′ methoxy ethyl-modified nucleoside, wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of HGFAC, wherein the oligonucleotide comprises an siRNA comprising a sense strand and an antisense strand, wherein the antisense strand comprises modification pattern 1AS: 5′-nsNfsnNfnNfnNfnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfhNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnnnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnnnNfnnNfnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfnNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 9AS: 5′-nsNfsnnnnnnnnnnnNfnnnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 10AS: 5′-nnnNfnNfnNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 12AS: 5′-nsNfsnnnNfnNfnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 13AS: 5′-nsNfsnnnNfnNfnNfnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 14AS: 5′-nsNfsnnnNfNfnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 15AS: 5′-nsNfsnnnnNfnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 16AS: 5′-nsNfsnnNfnNfnnNfnNfnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern: 5′-nsNfsnnNfnNfnnNfnnnNfnNfnNfnsnsn-3′, wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of HGFAC, wherein the oligonucleotide comprises an siRNA comprising a sense strand and an antisense strand, wherein the sense strand comprises pattern 1S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 2S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 3S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 4S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 5S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 6S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 7S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 8S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 9S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 10S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 11 S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 12S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 13S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 14S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 15S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 16S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 17S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 18S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 19S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 20S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 21S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 22S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 23S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 24S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 25S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 26S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 27S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 28S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 29S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 30S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 31S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 32S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 33S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 34S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 35S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 36S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 37S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 38S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 39S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 40S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 41S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 42S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 43S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 44S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 45S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 46S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 47S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 48S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 49S and the antisense strand comprises pattern 1AS, 2AS, 3AS, 4AS, 5AS, 6AS, 7AS, 8AS, 9AS, 10AS, 11AS, 12AS, 13AS, 14AS, 15AS, or 16AS. In some embodiments, the sense strand comprises pattern 50S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 51S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 52S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 53S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 54S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 55S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 56S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 57S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 58S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 59S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 60S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 61S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 62S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 63S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 64S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 65S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 66S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 67S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 68S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 69S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 70S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 71S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 72S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the sense strand comprises pattern 73S and the antisense strand comprises pattern 1AS, 2AS, 3AS, 4AS, 5AS, 6AS, 7AS, 8AS, 9AS, 10AS, 11 AS, 12AS, 13AS, 14AS, 15AS, or 16AS.

In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 1AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 2AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 3AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 4AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 5AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 6AS. In some embodiments, the sense strand comprises pattern 1S, 2S, 3S, 4S, 5S, 6S, 7S, 8S, 9S, 10S, 1S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 7AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 8AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 9AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 10AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 11 AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 12AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 13AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 14AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 15AS. In some embodiments, the sense strand comprises pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S and the antisense strand comprises pattern 16AS.

In some embodiments, the sense strand comprises modification pattern 1AS, 2AS, 3AS, 4AS, 5AS, 6AS, 7AS, 8AS, 9AS, 10AS, 11 AS, 12AS, 13AS, 14AS, 15AS, or 16AS. In some embodiments, the antisense strand comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, or 73S. In some embodiments, the sense strand or the antisense strand comprises modification pattern ASO1.

In some embodiments, purines of the sense strand comprise 2′ fluoro modified purines. In some embodiments, purines of the sense strand comprise 2′-O-methyl modified purines. In some embodiments, purines of the sense strand comprise a mixture of 2′ fluoro and 2′-O-methyl modified purines. In some embodiments, all purines of the sense strand comprise 2′ fluoro modified purines. In some embodiments, all purines of the sense strand comprise 2′-O-methyl modified purines. In some embodiments, all purines of the sense strand comprise a mixture of 2′ fluoro and 2′-O-methyl modified purines.

In some embodiments, pyrimidines of the sense strand comprise 2′ fluoro modified pyrimidines. In some embodiments, pyrimidines of the sense strand comprise 2′-O-methyl modified pyrimidines. In some embodiments, pyrimidines of the sense strand comprise a mixture of 2′ fluoro and 2′-O-methyl modified pyrimidines. In some embodiments, all pyrimidines of the sense strand comprise 2′ fluoro modified pyrimidines. In some embodiments, all pyrimidines of the sense strand comprise 2′-O-methyl modified pyrimidines. In some embodiments, all pyrimidines of the sense strand comprise a mixture of 2′ fluoro and 2′-O-methyl modified pyrimidines.

In some embodiments, purines of the sense strand comprise 2′ fluoro modified purines, and pyrimidines of the sense strand comprise a mixture of 2′ fluoro and 2′-O-methyl modified pyrimidines. In some embodiments, purines of the sense strand comprise 2′-O-methyl modified purines, and pyrimidines of the sense strand comprise a mixture of 2′ fluoro and 2′-O-methyl modified pyrimidines. In some embodiments, purines of the sense strand comprise 2′ fluoro modified purines, and pyrimidines of the sense strand comprise 2′-O-methyl modified pyrimidines. In some embodiments, purines of the sense strand comprise 2′-O-methyl modified purines, and pyrimidines of the sense strand comprise 2′ fluoro modified pyrimidines. In some embodiments, pyrimidines of the sense strand comprise 2′ fluoro modified pyrimidines, and purines of the sense strand comprise a mixture of 2′ fluoro and 2′-O-methyl modified purines. In some embodiments, pyrimidines of the sense strand comprise 2′-O-methyl modified pyrimidines, and purines of the sense strand comprise a mixture of 2′ fluoro and 2′-O-methyl modified purines. In some embodiments, pyrimidines of the sense strand comprise 2′ fluoro modified pyrimidines, and purines of the sense strand comprise 2′-O-methyl modified purines. In some embodiments, pyrimidines of the sense strand comprise 2′-O-methyl modified pyrimidines, and purines of the sense strand comprise 2′ fluoro modified purines.

In some embodiments, all purines of the sense strand comprise 2′ fluoro modified purines, and all pyrimidines of the sense strand comprise a mixture of 2′ fluoro and 2′-O-methyl modified pyrimidines. In some embodiments, all purines of the sense strand comprise 2′-O-methyl modified purines, and all pyrimidines of the sense strand comprise a mixture of 2′ fluoro and 2′-O-methyl modified pyrimidines. In some embodiments, all purines of the sense strand comprise 2′ fluoro modified purines, and all pyrimidines of the sense strand comprise 2′-O-methyl modified pyrimidines. In some embodiments, all purines of the sense strand comprise 2′-O-methyl modified purines, and all pyrimidines of the sense strand comprise 2′ fluoro modified pyrimidines. In some embodiments, all pyrimidines of the sense strand comprise 2′ fluoro modified pyrimidines, and all purines of the sense strand comprise a mixture of 2′ fluoro and 2′-O-methyl modified purines. In some embodiments, all pyrimidines of the sense strand comprise 2′-O-methyl modified pyrimidines, and all purines of the sense strand comprise a mixture of 2′ fluoro and 2′-O-methyl modified purines. In some embodiments, all pyrimidines of the sense strand comprise 2′ fluoro modified pyrimidines, and all purines of the sense strand comprise 2′-O-methyl modified purines. In some embodiments, all pyrimidines of the sense strand comprise 2′-O-methyl modified pyrimidines, and all purines of the sense strand comprise 2′ fluoro modified purines.

In some embodiments, purines of the antisense strand comprise 2′ fluoro modified purines. In some embodiments, purines of the antisense strand comprise 2′-O-methyl modified purines. In some embodiments, purines of the antisense strand comprise a mixture of 2′ fluoro and 2′-O-methyl modified purines. In some embodiments, all purines of the antisense strand comprise 2′ fluoro modified purines. In some embodiments, all purines of the antisense strand comprise 2′-O-methyl modified purines. In some embodiments, all purines of the antisense strand comprise a mixture of 2′ fluoro and 2′-O-methyl modified purines.

In some embodiments, pyrimidines of the antisense strand comprise 2′ fluoro modified pyrimidines. In some embodiments, pyrimidines of the antisense strand comprise 2′-O-methyl modified pyrimidines. In some embodiments, pyrimidines of the antisense strand comprise a mixture of 2′ fluoro and 2′-O-methyl modified pyrimidines. In some embodiments, all pyrimidines of the antisense strand comprise 2′ fluoro modified pyrimidines. In some embodiments, all pyrimidines of the antisense strand comprise 2′-O-methyl modified pyrimidines. In some embodiments, all pyrimidines of the antisense strand comprise a mixture of 2′ fluoro and 2′-O-methyl modified pyrimidines.

In some embodiments, purines of the antisense strand comprise 2′ fluoro modified purines, and pyrimidines of the antisense strand comprise a mixture of 2′ fluoro and 2′-O-methyl modified pyrimidines. In some embodiments, purines of the antisense strand comprise 2′-O-methyl modified purines, and pyrimidines of the antisense strand comprise a mixture of 2′ fluoro and 2′-O-methyl modified pyrimidines. In some embodiments, purines of the antisense strand comprise 2′ fluoro modified purines, and pyrimidines of the antisense strand comprise 2′-O-methyl modified pyrimidines. In some embodiments, purines of the antisense strand comprise 2′-O-methyl modified purines, and pyrimidines of the antisense strand comprise 2′ fluoro modified pyrimidines. In some embodiments, pyrimidines of the antisense strand comprise 2′ fluoro modified pyrimidines, and purines of the antisense strand comprise a mixture of 2′ fluoro and 2′-O-methyl modified purines. In some embodiments, pyrimidines of the antisense strand comprise 2′-O-methyl modified pyrimidines, and purines of the antisense strand comprise a mixture of 2′ fluoro and 2′-O-methyl modified purines. In some embodiments, pyrimidines of the antisense strand comprise 2′ fluoro modified pyrimidines, and purines of the antisense strand comprise 2′-O-methyl modified purines. In some embodiments, pyrimidines of the antisense strand comprise 2′-O-methyl modified pyrimidines, and purines of the antisense strand comprise 2′ fluoro modified purines.

In some embodiments, all purines of the antisense strand comprise 2′ fluoro modified purines, and all pyrimidines of the antisense strand comprise a mixture of 2′ fluoro and 2′-O-methyl modified pyrimidines. In some embodiments, all purines of the antisense strand comprise 2′-O-methyl modified purines, and all pyrimidines of the antisense strand comprise a mixture of 2′ fluoro and 2′-O-methyl modified pyrimidines. In some embodiments, all purines of the antisense strand comprise 2′ fluoro modified purines, and all pyrimidines of the antisense strand comprise 2′-O-methyl modified pyrimidines. In some embodiments, all purines of the antisense strand comprise 2′-O-methyl modified purines, and all pyrimidines of the antisense strand comprise 2′ fluoro modified pyrimidines. In some embodiments, all pyrimidines of the antisense strand comprise 2′ fluoro modified pyrimidines, and all purines of the antisense strand comprise a mixture of 2′ fluoro and 2′-O-methyl modified purines. In some embodiments, all pyrimidines of the antisense strand comprise 2′-O-methyl modified pyrimidines, and all purines of the antisense strand comprise a mixture of 2′ fluoro and 2′-O-methyl modified purines. In some embodiments, all pyrimidines of the antisense strand comprise 2′ fluoro modified pyrimidines, and all purines of the antisense strand comprise 2′-O-methyl modified purines. In some embodiments, all pyrimidines of the antisense strand comprise 2′-O-methyl modified pyrimidines, and all purines of the antisense strand comprise 2′ fluoro modified purines.

In some embodiments, the siRNA comprises the sense strand and/or the antisense strand sequence of an siRNA in Table F, or a nucleic acid sequence thereof having 3 or 4 nucleoside substitutions, additions, or deletions. In some embodiments, the siRNA comprises the sense strand and/or the antisense strand sequence of an siRNA in Table F, or a nucleic acid sequence thereof having 1 or 2 nucleoside substitutions, additions, or deletions. In some embodiments, the siRNA comprises the sense strand and/or the antisense strand sequence of an siRNA in Table F. The siRNA may include the same internucleoside linkage modifications or nucleoside modifications as those in Table F. The siRNA may include any different internucleoside linkage modifications or nucleoside modifications different from those in Table F. The siRNA may include some unmodified internucleoside linkages or nucleosides.

In some embodiments, the siRNA comprises the sense strand and/or the antisense strand sequence of an siRNA of subset F, or a nucleic acid sequence thereof having 3 or 4 nucleoside substitutions, additions, or deletions. In some embodiments, the siRNA comprises the sense strand and/or the antisense strand sequence of an siRNA of subset F, or a nucleic acid sequence thereof having 1 or 2 nucleoside substitutions, additions, or deletions. In some embodiments, the siRNA comprises the sense strand and/or the antisense strand sequence of an siRNA of subset F. In some embodiments, the siRNA is cross-reactive with a non-human primate (NHP) HGFAC mRNA. The siRNA may include one or more internucleoside linkages and/or one or more nucleoside modifications.

In some embodiments, the siRNA comprises the sense strand and/or the antisense strand sequence of an siRNA in Table G(2), or a nucleic acid sequence thereof having 3 or 4 nucleoside substitutions, additions, or deletions. In some embodiments, the siRNA comprises the sense strand and/or the antisense strand sequence of an siRNA in Table G(2), or a nucleic acid sequence thereof having 1 or 2 nucleoside substitutions, additions, or deletions. In some embodiments, the siRNA comprises the sense strand and/or the antisense strand sequence of an siRNA in Table G(2). The siRNA may include the same internucleoside linkage modifications or nucleoside modifications as those in Table G(2). The siRNA may include any different internucleoside linkage modifications or nucleoside modifications different from those in Table G(2). The siRNA may include some unmodified internucleoside linkages or nucleosides.

In some embodiments, the siRNA comprises the sense strand and/or the antisense strand sequence of an siRNA in Table G(3), or a nucleic acid sequence thereof having 3 or 4 nucleoside substitutions, additions, or deletions. In some embodiments, the siRNA comprises the sense strand and/or the antisense strand sequence of an siRNA in Table G(3), or a nucleic acid sequence thereof having 1 or 2 nucleoside substitutions, additions, or deletions. In some embodiments, the siRNA comprises the sense strand and/or the antisense strand sequence of an siRNA in Table G(3). The siRNA may include the same internucleoside linkage modifications or nucleoside modifications as those in Table G(3). The siRNA may include any different internucleoside linkage modifications or nucleoside modifications different from those in Table G(3). The siRNA may include some unmodified internucleoside linkages or nucleosides.

In some embodiments, the siRNA comprises the sense strand and/or the antisense strand sequence of an siRNA in Table H(2)), or a nucleic acid sequence thereof having 3 or 4 nucleoside substitutions, additions, or deletions. In some embodiments, the siRNA comprises the sense strand and/or the antisense strand sequence of an siRNA in Table H(2)), or a nucleic acid sequence thereof having 1 or 2 nucleoside substitutions, additions, or deletions. In some embodiments, the siRNA comprises the sense strand and/or the antisense strand sequence of an siRNA in Table H(2)). The siRNA may include the same internucleoside linkage modifications or nucleoside modifications as those in Table H(2)). The siRNA may include any different internucleoside linkage modifications or nucleoside modifications different from those in Table H(2)). The siRNA may include some unmodified internucleoside linkages or nucleosides.

In some embodiments, the siRNA comprises the sense strand and/or the antisense strand sequence of an siRNA in Table 4, or a nucleic acid sequence thereof having 3 or 4 nucleoside substitutions, additions, or deletions. In some embodiments, the siRNA comprises the sense strand and/or the antisense strand sequence of an siRNA in Table 4, or a nucleic acid sequence thereof having 1 or 2 nucleoside substitutions, additions, or deletions. In some embodiments, the siRNA comprises the sense strand and/or the antisense strand sequence of an siRNA in Table 4. The siRNA may include the same internucleoside linkage modifications or nucleoside modifications as those in Table 4. The siRNA may include any different internucleoside linkage modifications or nucleoside modifications different from those in Table 4. The siRNA may include some unmodified internucleoside linkages or nucleosides.

In some embodiments, the siRNA comprises the sense strand and/or the antisense strand sequence of an siRNA in Table 10, or a nucleic acid sequence thereof having 3 or 4 nucleoside substitutions, additions, or deletions. In some embodiments, the siRNA comprises the sense strand and/or the antisense strand sequence of an siRNA in Table 10, or a nucleic acid sequence thereof having 1 or 2 nucleoside substitutions, additions, or deletions. In some embodiments, the siRNA comprises the sense strand and/or the antisense strand sequence of an siRNA in Table 10. The siRNA may include the same internucleoside linkage modifications or nucleoside modifications as those in Table 10. The siRNA may include any different internucleoside linkage modifications or nucleoside modifications different from those in Table 10. The siRNA may include some unmodified internucleoside linkages or nucleosides.

In some embodiments, the siRNA comprises the sense strand and/or the antisense strand sequence of an siRNA in Table 13, or a nucleic acid sequence thereof having 3 or 4 nucleoside substitutions, additions, or deletions. In some embodiments, the siRNA comprises the sense strand and/or the antisense strand sequence of an siRNA in Table 13, or a nucleic acid sequence thereof having 1 or 2 nucleoside substitutions, additions, or deletions. In some embodiments, the siRNA comprises the sense strand and/or the antisense strand sequence of an siRNA in Table 13. The siRNA may include the same internucleoside linkage modifications or nucleoside modifications as those in Table 13. The siRNA may include any different internucleoside linkage modifications or nucleoside modifications different from those in Table 13. The siRNA may include some unmodified internucleoside linkages or nucleosides.

Disclosed herein, in some embodiments, are modified oligonucleotides. The modified oligonucleotide may be an siRNA that includes modifications to the ribose rings, and phosphate linkages. The modifications may be in particular patterns that maximize cell delivery, stability, and efficiency. The siRNA may also include a vinyl phosphonate and a hydrophobic group. These modifications may aid in delivery to a cell or tissue within a subject. The modified oligonucleotide may be used in a method such as a treatment method or a method of reducing gene expression.

In some embodiments, the oligonucleotide comprises a duplex consisting of 21 nucleotide single strands with base pairing between 19 of the base pairs. In some embodiments, the duplex comprises single-stranded 2 nucleotide overhangs are at the 3′ ends of each strand. One strand (antisense strand) is complementary to a HGFAC mRNA. Each end of the antisense strand has one to two phosphorothioate bonds. The 5′ end has an optional phosphate mimic such as a vinyl phosphonate. In some embodiments, the oligonucleotide is used to knock down a HGFAC mRNA or a target protein. In some embodiments, the sense strand has the same sequence as the HGFAC mRNA. In some embodiments, there are 1-2 phosphorothioates at the 3′ end. In some embodiments, there are 1 or no phosphorothioates at the 5′ end. In some embodiments, there is a hydrophobic conjugate of 12 to 25 carbons attached at the 5′ end via a phosphodiester bond.

In some cases, the sense strand of any of the siRNAs comprises siRNA with a particular modification pattern. In some embodiments of the modification pattern, position 9 counting from the 5′ end of the sense strand may have a 2′F modification. In some embodiments, when position 9 of the sense strand is a pyrimidine, then all purines in the sense strand have a 2′OMe modification. In some embodiments, when position 9 is the only pyrimidine between positions 5 and 11 of the sense stand, then position 9 is the only position with a 2′F modification in the sense strand. In some embodiments, when position 9 and only one other base between positions 5 and 11 of the sense strand are pyrimidines, then both of these pyrimidines are the only two positions with a 2′F modification in the sense strand. In some embodiments, when position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. In some embodiments, when there are more than 2 pyrimidines between positions 5 and 11 of the sense strand, then all combinations of pyrimidines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some embodiments, when position 9 of the sense strand is a purine, then all purines in the sense strand have a 2′OMe modification. In some embodiments, when position 9 is the only purine between positions 5 and 11 of the sense stand, then position 9 is the only position with a 2′F modification in the sense strand. In some embodiments, when position 9 and only one other base between positions 5 and 11 of the sense strand are purines, then both of these purines are the only two positions with a 2′F modification in the sense strand. In some embodiments, when position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. In some embodiments, when there are more than 2 purines between positions 5 and 11 of the sense strand, then all combinations of purines having the 2′F modification are allowed that have three to five 2′F modifications in total, provided that the sense strand does not have three 2′F modifications in a row. In some cases, the sense strand of any of the siRNAs comprises a modification pattern which conforms to any or all of these sense strand rules.

In some cases, position 9 of the sense strand can be a 2′deoxy. In these cases, 2′F and 2′OMe modifications may occur at the other positions of the sense strand. In some cases, the sense strand of any of the siRNAs comprises a modification pattern which conforms to these sense strand rules.

In some cases, the sense strand of any of the siRNAs comprises a modification pattern which conforms to these sense strand rules.

Disclosed herein, in some embodiments are compositions comprising an oligonucleotide that targets HGFAC and when administered to a cell decreases expression of HGFAC, wherein the oligonucleotide comprises a small interfering RNA (siRNA) comprising a sense strand and an antisense strand, wherein the sense strand comprises a sense strand sequence described herein in which at least one internucleoside linkage is modified and at least one nucleoside is modified, or an sense strand sequence comprising 1 or 2 nucleoside substitutions, additions, or deletions of the oligonucleotide sequence in which at least one internucleoside linkage is modified and at least one nucleoside is modified, and wherein the antisense strand comprises an antisense strand sequence described herein in which at least one internucleoside linkage is modified and at least one nucleoside is modified, or an oligonucleotide sequence comprising 1 or 2 nucleoside substitutions, additions, or deletions of the antisense strand sequence in which at least one internucleoside linkage is modified and at least one nucleoside is modified.

Some embodiments relate to methods that include administering the composition to a subject.

In some embodiments, the siRNA comprises the sense strand comprising any one of SEQ ID NO: 4804-4813, or a nucleic acid sequence thereof having 3 or 4 nucleoside substitutions, additions, or deletions. In some embodiments, the siRNA comprises the sense strand comprising any one of SEQ ID NO: 4804-4813, or a nucleic acid sequence thereof having 1 or 2 nucleoside substitutions, additions, or deletions. In some embodiments, the siRNA comprises the sense strand comprising any one of SEQ ID NO: 4804-4813. The siRNA may include the same internucleoside linkage modifications or nucleoside modifications as any one of SEQ ID NO: 4804-4813. The siRNA may include any different internucleoside linkage modifications or nucleoside modifications different from any one of SEQ ID NO: 4804-4813. The siRNA may include some unmodified internucleoside linkages or nucleosides.

In some embodiments, the siRNA comprises the antisense strand comprising any one of SEQ ID NO: 4814-4821, or a nucleic acid sequence thereof having 3 or 4 nucleoside substitutions, additions, or deletions. In some embodiments, the siRNA comprises the antisense strand comprising any one of SEQ ID NO: 4814-4821, or a nucleic acid sequence thereof having 1 or 2 nucleoside substitutions, additions, or deletions. In some embodiments, the siRNA comprises the antisense strand comprising any one of SEQ ID NO: 4814-4821. The siRNA may include the same internucleoside linkage modifications or nucleoside modifications as any one of SEQ ID NO: 4814-4821. The siRNA may include any different internucleoside linkage modifications or nucleoside modifications different from any one of SEQ ID NO: 4814-4821. The siRNA may include some unmodified internucleoside linkages or nucleosides.

### 4. Modified ASOs

In some embodiments, the composition comprises an oligonucleotide that inhibits the expression of HGFAC, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′, wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 38S, 39S, 40S, 41S, 42S, 43S, 44S, 45S, 46S, 47S, 48S, 49S, 50S, 51S, 52S, 53S, 54S, 55S, 56S, 57S, 58S, 59S, 60S, 61S, 62S, 63S, 64S, 65S, 66S, 67S, 68S, 69S, 70S, 71S, 72S, 73S, 1AS, 2AS, 3AS, 4AS, 5AS, 6AS, 7AS, 8AS, 9AS, 10AS, 11AS, 12AS, 13AS, 14AS, 15AS, or 16AS.

### D. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### II. Methods and Uses

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject. The disorder may comprise cancer.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

In some embodiments, the administration is systemic. In some embodiments, the administration is intravenous. In some embodiments, the administration is by injection.

In some embodiments, the subject is administered the HGFAC inhibitor described herein as part of a combined treatment with another therapy. In some embodiments, the combination therapy includes a checkpoint inhibitor. In some embodiments, the checkpoint inhibitor includes as a PDL1 inhibitor. In some embodiments, the checkpoint inhibitor includes a PD1 inhibitor. In some embodiments, the checkpoint inhibitor includes a CTLA4 inhibitor. In some embodiments, the PDL1 inhibitor comprises atezolizumab, avelumab, durvalumab, KN035, CK-301, AUNP12, CA-170, BMS-986189, or a combination thereof. In some embodiments, the PD-1 inhibitor comprises nivolumab, pembrolizumab, cemiplimab, dorstarlimab, JTX-4014, spartalizumab (PDR001), camrelizumab (SHR1210), sintilmab (1B1308), tislelizumab (BGB-A317), toripalimab (JS 001), INCMGA00012 (MGA012), AMP-224, AMP-514, or combinations thereof. In some embodiments, the CTLA4 inhibitor comprises Ipilimumab (Yervoy), tremelimumab (Imjuno), or combinations thereof. In some embodiments, the HGFAC inhibitor and the checkpoint inhibitor are administered at the same time. In some embodiments, the HGFAC inhibitor and the PDL1 inhibitor are administered simultaneously. In some embodiments, the HGFAC inhibitor and the PDL1 inhibitor are administered substantially simultaneously. In some embodiments, the HGFAC inhibitor and the PDL1 inhibitor are administered sequentially. In some embodiments, the HGFAC inhibitor and the PDL1 inhibitor are administered separately. In some embodiments, the combination therapy includes radiotherapy. In some embodiments, the HGFAC inhibitor and the radiotherapy are administered at the same time. In some embodiments, the HGFAC inhibitor and the radiotherapy are administered simultaneously. In some embodiments, the HGFAC inhibitor and the radiotherapy are administered substantially simultaneously. In some embodiments, the HGFAC inhibitor and the radiotherapy are administered sequentially. In some embodiments, the HGFAC inhibitor and the radiotherapy are administered separately.

### A. Cancers

Some embodiments of the methods described herein include treating a disorder such as cancer in a subject in need thereof. Non-limiting examples of cancer may include: malignant neoplasms, solid tumors, hematological cancers, malignant neoplasms of urinary tract, malignant neoplasms of thyroid and other endocrine glands, malignant neoplasms of soft tissue, malignant neoplasms of skin, malignant neoplasms of skeletal system, malignant neoplasms of respiratory and intrathoracic organs, malignant neoplasms of male genital organs, malignant neoplasms of female genital organs, malignant neoplasms of lip, oral cavity and pharynx, malignant neoplasms of eye, brain and other parts of central nervous system, malignant neoplasms of digestive system, malignant neoplasms of breast, malignant neoplasms of pancreas, malignant neoplasms of liver, or malignant melanoma. Any one of these cancers, or any grouping, may be treated by a method or composition described herein. In some embodiments, the method modulates an immune response that may affect the cancer. In some embodiments, the method increases an immune response against cancer.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female.

In some embodiments, the subject is an adult (e.g., at least 18 years old). In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline clinical response measurement, a baseline cell count measurement, a baseline antibody level measurement, or a baseline tissue marker level measurement, a baseline HGFAC protein measurement, or a baseline HGFAC mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device.

In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g., HPLC) assay. In some embodiments, the baseline measurement is obtained by PCR.

In some embodiments, the baseline measurement is a baseline clinical response measurement. Non-limiting examples of clinical response baseline measurements include: immune specific related response criteria (irRC) such as set forth in iRECIST, progression free survival (PFS), duration of response (DOR), disease control rate (DCR), health-related quality of life, milestone survival, clinical benefit rate, pathological complete response, complete response, objective response rate, duration of clinical benefit, time to next treatment, time to treatment failure, disease-free survival, or time to progression. In some embodiments, the baseline clinical response measurement is obtained through patient observation or patient response. In some embodiments, the baseline clinical response measurement is obtained by observation of a patient or discussion with a patient.

In some embodiments, the baseline measurement is a baseline cell count measurement. The baseline cell count measurement may include a baseline immune cell count measurement. Non-limiting examples of cell count baseline measurements may include: myeloid derived suppressor cell (MDSC) counts and subpopulations, CD8+ tumor infiltrating lymphocytes (TILs), leukocyte counts, T and B lymphocyte counts and activation states, monocyte counts, macrophage counts and activation states, dendritic cell counts, neutrophil counts, eosinophil counts, basophil counts, or mast cell counts. In some embodiments, the baseline cell count measurement is a baseline cell count concentration (for example, cells per liter). In some embodiments, the baseline cell count concentration is a baseline total cell count concentration. In some embodiments, the baseline cell count measurement is a baseline circulating cell count measurement. In some embodiments, the baseline cell count measurement is obtained by centrifuging a blood sample and measuring the sample in various concentrations.

In some embodiments, the baseline measurement is a baseline antibody level measurement. Non-limiting examples of cell count baseline measurements include: IgA levels, IgG levels, or IgM. In some embodiments, the baseline antibody level measurement is a baseline antibody level concentration (for example, mg/dL). In some embodiments, the baseline antibody measurement is a baseline circulating antibody measurement. In some embodiments, the baseline antibody measurement is obtained by an assay such as an immunoassay, a colorimetric assay, or a fluorescence assay.

In some embodiments, the baseline measurement is a baseline tumor marker level measurement. Non-limiting examples of cell count baseline measurements include levels of tumor markers such as CEA, PSA, CA 125, CA 15-3, CA 19-9, CA 27.29, CA 72-4, AFP, hCG, B2M, BTA, Calcitonin, CgA, CELLSEARCH, DCP, Gastrin, HE4, LDH, NSE, NMP22, or PAP. In some embodiments, the baseline tumor marker level measurement is a baseline tumor marker level concentration (for example, mg/dL). In some embodiments, the baseline tumor marker level concentration is a baseline total tumor marker level concentration. In some embodiments, the baseline tumor marker level measurement is a baseline circulating tumor marker level measurement. In some embodiments, the baseline tumor marker level measurement is obtained by a blood test, urine test, or biopsy.

In some embodiments, the baseline measurement is a baseline HGFAC protein measurement. In some embodiments, the baseline HGFAC protein measurement comprises a baseline HGFAC protein level. In some embodiments, the baseline HGFAC protein level is indicated as a mass or percentage of HGFAC protein per sample weight. In some embodiments, the baseline HGFAC protein level is indicated as a mass or percentage of HGFAC protein per sample volume. In some embodiments, the baseline HGFAC protein level is indicated as a mass or percentage of HGFAC protein per total protein within the sample. In some embodiments, the baseline HGFAC protein measurement is a baseline circulating/tissue HGFAC protein measurement. In some embodiments, the baseline HGFAC protein measurement is obtained by an assay such as an immunoassay, a colorimetric assay, or a fluorescence assay.

In some embodiments, the baseline measurement is a baseline HGFAC mRNA measurement. In some embodiments, the baseline HGFAC mRNA measurement comprises a baseline HGFAC mRNA level. In some embodiments, the baseline HGFAC mRNA level is indicated as an amount or percentage of HGFAC mRNA per sample weight. In some embodiments, the baseline HGFAC mRNA level is indicated as an amount or percentage of HGFAC mRNA per sample volume. In some embodiments, the baseline HGFAC mRNA level is indicated as an amount or percentage of HGFAC mRNA per total mRNA within the sample. In some embodiments, the baseline HGFAC mRNA level is indicated as an amount or percentage of HGFAC mRNA per total nucleic acids within the sample. In some embodiments, the baseline HGFAC mRNA level is indicated relative to another mRNA level, such as an mRNA level of a housekeeping gene, within the sample. In some embodiments, the baseline HGFAC mRNA measurement is a baseline tissue HGFAC mRNA measurement. In some embodiments, the baseline HGFAC mRNA measurement is obtained by an assay such as a polymerase chain reaction (PCR) assay. In some embodiments, the PCR comprises quantitative PCR (qPCR). In some embodiments, the PCR comprises reverse transcription of the HGFAC mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. A blood sample may be a plasma sample. In some embodiments, the sample comprises serum. In some embodiments, the sample is a serum sample. A blood sample may be a serum sample.

In some embodiments, the sample comprises a tissue. In some embodiments, the sample is a tissue sample. In some embodiments, the tissue comprises liver or cancer tissue. For example, the baseline HGFAC mRNA measurement, or the baseline HGFAC protein measurement, may be obtained in a liver sample obtained from the patient. In some embodiments, the tissue comprises liver tissue. The liver may include hepatocytes. In some embodiments, the tissue comprises cancer tissue.

In some embodiments, the sample includes cells. In some embodiments, the sample comprises a cell. In some embodiments, the cell comprises a liver cell or a cancer cell. In some embodiments, the cell is a liver cell. In some embodiments, the liver cell is a hepatocyte. In some embodiments, the cell is a cancer cell.

### D. Effects

In some embodiments, the composition or administration of the composition affects a measurement such as a clinical response measurement, a cell count measurement, an antibody level measurement, a tumor marker level measurement, an HGFAC protein measurement, or an HGFAC mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the cancer has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, a chromatography (e.g., HPLC) assay, or a PCR assay. In some embodiments, the measurement is obtained by an assay such as an immunoassay, a colorimetric assay, a fluorescence assay, or a chromatography (e.g., HPLC) assay.

In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. For example, an adverse phenotype of cancer may be reduced upon administration of the composition. In some embodiments, the reduction is measured in a second sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the measurement relative to the baseline measurement. For example, a protective cancer phenotype may be increased upon administration of the composition. In some embodiments, the increase is measured in a second sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a clinical response measurement. The clinical response measurement may include a time. The clinical response measurement may include an amount. The clinical response measurement may include a rate. Non-limiting examples of clinical response measurements include: immune specific related response criteria (irRC) such as set forth in iRECIST, progression free survival (PFS), duration of response (DOR), disease control rate (DCR), health-related quality of life, milestone survival, clinical benefit rate, pathological complete response, complete response, objective response rate, duration of clinical benefit, time to next treatment, time to treatment failure, disease-free survival, or time to progression. In some embodiments, the clinical response measurement is obtained through patient observation or patient response. In some embodiments, the clinical response measurement is a circulating clinical response measurement. In some embodiments, the clinical response measurement is obtained by observation of a patient. The clinical response measurement may include irRC. The clinical response measurement may include PFS. The clinical response measurement may include DOR. The clinical response measurement may include DCR. The clinical response measurement may include health-related quality of life. The clinical response measurement may include milestone survival. The clinical response measurement may include clinical benefit rate. The clinical response measurement may include pathological complete response. The clinical response measurement may include complete response. The clinical response measurement may include objective response rate. The clinical response measurement may include duration of clinical benefit. The clinical response measurement may include time to next treatment. The clinical response measurement may include time to treatment failure. The clinical response measurement may include disease-free survival. The clinical response measurement may include time to progression.

In some embodiments, the composition increases the clinical response measurement relative to the baseline clinical response measurement. For example, a beneficial effect in the clinical response may be increased upon administration of the composition. In some embodiments, the clinical response measurement is measured in a second sample obtained from the subject after administering the composition to the subject. In some embodiments, the clinical response measurement is measured directly in the subject after administering the composition to the subject. In some embodiments, the clinical response measurement is increased by about 2.5% or more, about 5% or more, or about 7.5% or more, relative to the baseline measurement. In some embodiments, the clinical response measurement is increased by about 10% or more, relative to the baseline measurement. In some embodiments, the clinical response measurement is increased by about 20% or more, about 30% or more, about 40% or more, about 50% or more, about 60% or more, about 70% or more, about 80% or more, about 90% or more, relative to the baseline measurement. In some embodiments, the clinical response measurement is increased by about 100% or more, increased by about 250% or more, increased by about 500% or more, increased by about 750% or more, or increased by about 1000% or more, relative to the baseline measurement. In some embodiments, the clinical response measurement is increased by no more than about 2.5%, no more than about 5%, or no more than about 7.5%, relative to the baseline measurement. In some embodiments, the clinical response measurement is increased by no more than about 10%, relative to the baseline measurement. In some embodiments, the clinical response measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the clinical response measurement is increased by no more than about 100%, increased by no more than about 250%, increased by no more than about 500%, increased by no more than about 750%, or increased by no more than about 1000%, relative to the baseline measurement. In some embodiments, the clinical response measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a cell count measurement. The cell count measurement may include an immune cell count measurement. Non-limiting examples of cell count measurements include: myeloid derived suppressor cell (MDSC) counts and subpopulations, CD8+ tumor infiltrating lymphocytes (TILs), Leukocyte counts, T and B lymphocyte counts and activation states, monocyte counts, macrophage counts and activation states, dendritic cell counts, neutrophil counts, eosinophil counts, basophil counts, or mast cell counts. The cell count measurement may include a MDSC count. The cell count measurement may include a MDSC subpopulation measurement. The cell count measurement may include a CD8+ TIL measurement. The cell count measurement may include a leukocyte count. The cell count measurement may include a T lymphocyte count. The cell count measurement may include a B lymphocyte count. The cell count measurement may include a T lymphocyte activation state measurement. The cell count measurement may include a B lymphocyte activation state measurement. The cell count measurement may include a monocyte count. The cell count measurement may include a macrophage count. The cell count measurement may include a macrophage activation state measurement. The cell count measurement may include a dendritic cell count. The cell count measurement may include a neutrophil count. The cell count measurement may include a eosinophil count. The cell count measurement may include a basophil count. The cell count measurement may include a mast cell count. In some embodiments, the cell count measurement is a cell count concentration (for example, mg/dL). In some embodiments, the cell count measurement is a circulating cell count measurement in the blood. In some embodiments, the cell count measurement is obtained by centrifuging a blood sample and measuring the sample in various concentrations.

In some embodiments, the composition improves the cell count measurement relative to the baseline cell count measurement. The improvement may comprise a change (e.g., an increase or decrease). In some embodiments, the improvement is an increase. In some embodiments, the improvement is a decrease. In some embodiments, the composition improves circulating cell count relative to the baseline cell count measurement. In some embodiments, the improved cell counts are measured in a second sample obtained from the subject after administering the composition to the subject. In some embodiments, the cell count measurement is improved by about 2.5% or more, about 5% or more, or about 7.5% or more, relative to the baseline cell count measurement. In some embodiments, the cell count measurement is improved by about 10% or more, relative to the baseline cell count measurement. In some embodiments, the cell count measurement is improved by about 20% or more, about 30% or more, about 40% or more, about 50% or more, about 60% or more, about 70% or more, about 80% or more, about 90% or more, or about 100% or more relative to the baseline cell count measurement. In some embodiments, the cell count measurement is improved by no more than about 2.5%, no more than about 5%, or no more than about 7.5%, relative to the baseline cell count measurement. In some embodiments, the cell count measurement is improved by no more than about 10%, relative to the baseline cell count measurement. In some embodiments, the cell count measurement is improved by no more than about 20%, no more than about 30%, no more than about 40%, no more than about 50%, no more than about 60%, no more than about 70%, no more than about 80%, no more than about 90%, or about 100% relative to the baseline cell count measurement. In some embodiments, the cell count measurement is improved by 2.5%, 5%, 7.5%, 10%, 20%, 30%, 40%, 50%, 60%, 70%, 80%, 90%, or 100%, or by a range defined by any of the two aforementioned percentages. In some embodiments where the improvement is an increase, the change is by more than 100%.

In some embodiments, the measurement is an antibody level measurement. Non-limiting examples of antibody level measurements include: IgA levels, IgG levels, or IgM levels. In some embodiments, the antibody level measurement is an antibody level concentration (for example, mg/dL). The antibody level may include an IgA level. The antibody level may include an IgG level. The antibody level may include an IgM level. In some embodiments, the antibody level measurement is a circulating antibody level measurement. In some embodiments, the antibody level measurement is obtained by an assay such as an immunoassay, a colorimetric assay, or a fluorescence assay.

In some embodiments, the composition improves the antibody level measurement relative to the baseline antibody level measurement. The improvement may comprise a change (e.g., an increase or decrease). In some embodiments, the improvement is an increase. In some embodiments, the improvement is a decrease. In some embodiments, the composition improves circulating antibody level relative to the baseline antibody level measurement. In some embodiments, the improved antibody levels are measured in a second sample obtained from the subject after administering the composition to the subject. In some embodiments, the antibody level measurement is improved by about 2.5% or more, about 5% or more, or about 7.5% or more, relative to the baseline antibody level measurement. In some embodiments, the antibody level measurement is improved by about 10% or more, relative to the baseline antibody level measurement. In some embodiments, the antibody level measurement is improved by about 20% or more, about 30% or more, about 40% or more, about 50% or more, about 60% or more, about 70% or more, about 80% or more, about 90% or more, or about 100% or more relative to the baseline antibody level measurement. In some embodiments, the antibody level measurement is improved by no more than about 2.5%, no more than about 5%, or no more than about 7.5%, relative to the baseline antibody level measurement. In some embodiments, the antibody level measurement is improved by no more than about 10%, relative to the baseline antibody level measurement. In some embodiments, the antibody level measurement is improved by no more than about 20%, no more than about 30%, no more than about 40%, no more than about 50%, no more than about 60%, no more than about 70%, no more than about 80%, no more than about 90%, or about 100% relative to the baseline antibody level measurement. In some embodiments, the antibody level measurement is improved by 2.5%, 5%, 7.5%, 10%, 20%, 30%, 40%, 50%, 60%, 70%, 80%, 90%, or 100%, or by a range defined by any of the two aforementioned percentages. In some embodiments where the improvement is an increase, the change is by more than 100%.

In some embodiments, the measurement is a tumor marker level measurement. Non-limiting examples of tumor marker level measurements include levels of tumor markers such as CEA, PSA, CA 125, CA 15-3, CA 19-9, CA 27.29, CA 72-4, AFP, hCG, B2M, BTA, calcitonin, CgA, CELLSEARCH, DCP, gastrin, HE4, LDH, NSE, NMP22, or PAP. The tumor marker may include CEA. The tumor marker may include PSA. The tumor marker may include CA 125. The tumor marker may include CA 15-3. The tumor marker may include CA 19-9. The tumor marker may include CA 27.29. The tumor marker may include CA 72-4. The tumor marker may include AFP. The tumor marker may include hCG. The tumor marker may include B2M. The tumor marker may include BTA. The tumor marker may include calcitonin. The tumor marker may include CgA. The tumor marker may include CELLSEARCH. The tumor marker may include DCP. The tumor marker may include gastrin. The tumor marker may include HE4. The tumor marker may include LDH. The tumor marker may include NSE. The tumor marker may include NMP22. The tumor marker may include PAP. In some embodiments, the tumor marker level measurement is a tumor marker level concentration (for example, mg/dL). In some embodiments, the tumor marker level concentration is a total tumor marker level concentration. In some embodiments, the tumor marker level measurement is a circulating tumor marker level measurement. In some embodiments, the tumor marker level measurement is obtained by a blood test, urine test, or biopsy.

In some embodiments, the composition improves the tumor marker level measurement relative to the baseline tumor marker level measurement. The improvement may comprise a change (e.g., an increase or decrease). In some embodiments, the improvement is an increase. In some embodiments, the improvement is a decrease. In some embodiments, the composition improves circulating tumor marker level relative to the baseline tumor marker level measurement. In some embodiments, the improved tumor marker levels are measured in a second sample obtained from the subject after administering the composition to the subject. In some embodiments, the tumor marker level measurement is improved by about 2.5% or more, about 5% or more, or about 7.5% or more, relative to the baseline tumor marker level measurement. In some embodiments, the tumor marker level measurement is improved by about 10% or more, relative to the baseline tumor marker level measurement. In some embodiments, the tumor marker level measurement is improved by about 20% or more, about 30% or more, about 40% or more, about 50% or more, about 60% or more, about 70% or more, about 80% or more, about 90% or more, or about 100% or more relative to the baseline tumor marker level measurement. In some embodiments, the tumor marker level measurement is improved by no more than about 2.5%, no more than about 5%, or no more than about 7.5%, relative to the baseline tumor marker level measurement. In some embodiments, the tumor marker level measurement is improved by no more than about 10%, relative to the baseline tumor marker level measurement. In some embodiments, the tumor marker level measurement is improved by no more than about 20%, no more than about 30%, no more than about 40%, no more than about 50%, no more than about 60%, no more than about 70%, no more than about 80%, no more than about 90%, or about 100% relative to the baseline tumor marker level measurement. In some embodiments, the tumor marker level measurement is improved by 2.5%, 5%, 7.5%, 10%, 20%, 30%, 40%, 50%, 60%, 70%, 80%, 90%, or 100%, or by a range defined by any of the two aforementioned percentages. In some embodiments where the improvement is an increase, the change is by more than 100%.

In some embodiments, the measurement is an HGFAC protein measurement. In some embodiments, the HGFAC protein measurement comprises an HGFAC protein level. In some embodiments, the HGFAC protein level is indicated as a mass or percentage of HGFAC protein per sample weight. In some embodiments, the HGFAC protein level is indicated as a mass or percentage of HGFAC protein per sample volume. In some embodiments, the HGFAC protein level is indicated as a mass or percentage of HGFAC protein per total protein within the sample. In some embodiments, the HGFAC protein measurement is a circulating/tissue HGFAC protein measurement. In some embodiments, the HGFAC protein measurement is obtained by an assay such as an immunoassay, a colorimetric assay, or a fluorescence assay.

In some embodiments, the composition reduces the HGFAC protein measurement relative to the baseline HGFAC protein measurement. In some embodiments, the composition reduces circulating HGFAC protein levels relative to the baseline HGFAC protein measurement. In some embodiments, the composition reduces tissue HGFAC protein levels relative to the baseline HGFAC protein measurement.

In some embodiments, the reduced HGFAC protein levels are measured in a second sample obtained from the subject after administering the composition to the subject. In some embodiments, the HGFAC protein measurement is decreased by about 2.5% or more, about 5% or more, or about 7.5% or more, relative to the baseline HGFAC protein measurement. In some embodiments, the HGFAC protein measurement is decreased by about 10% or more, relative to the baseline HGFAC protein measurement. In some embodiments, the HGFAC protein measurement is decreased by about 20% or more, about 30% or more, about 40% or more, about 50% or more, about 60% or more, about 70% or more, about 80% or more, about 90% or more, or about 100%, relative to the baseline HGFAC protein measurement. In some embodiments, the HGFAC protein measurement is decreased by no more than about 2.5%, no more than about 5%, or no more than about 7.5%, relative to the baseline HGFAC protein measurement. In some embodiments, the HGFAC protein measurement is decreased by no more than about 10%, relative to the baseline HGFAC protein measurement. In some embodiments, the HGFAC protein measurement is decreased by no more than about 20%, no more than about 30%, no more than about 40%, no more than about 50%, no more than about 60%, no more than about 70%, no more than about 80%, no more than about 90%, or no more than about 100% relative to the baseline HGFAC protein measurement. In some embodiments, the HGFAC protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is an HGFAC mRNA measurement. In some embodiments, the HGFAC mRNA measurement comprises an HGFAC mRNA level. In some embodiments, the HGFAC mRNA level is indicated as an amount or percentage of HGFAC mRNA per sample weight. In some embodiments, the HGFAC mRNA level is indicated as an amount or percentage of HGFAC mRNA per sample volume. In some embodiments, the HGFAC mRNA level is indicated as an amount or percentage of HGFAC mRNA per total mRNA within the sample. In some embodiments, the HGFAC mRNA level is indicated as an amount or percentage of HGFAC mRNA per total nucleic acids within the sample. In some embodiments, the HGFAC mRNA level is indicated relative to another mRNA level, such as an mRNA level of a housekeeping gene, within the sample. In some embodiments, the HGFAC mRNA measurement is a circulating/tissue HGFAC mRNA measurement. In some embodiments, the HGFAC mRNA measurement is obtained by an assay such as a PCR assay. In some embodiments, the PCR comprises qPCR. In some embodiments, the PCR comprises reverse transcription of the HGFAC mRNA.

In some embodiments, the composition reduces the HGFAC mRNA measurement relative to the baseline HGFAC mRNA measurement. In some embodiments, the HGFAC mRNA measurement is obtained in a second sample obtained from the subject after administering the composition to the subject.

In some embodiments, the composition reduces HGFAC mRNA levels relative to the baseline HGFAC mRNA levels. In some embodiments, the reduced HGFAC mRNA levels are measured in a second sample obtained from the subject after administering the composition to the subject. In some embodiments, the second sample is a liver sample. In some embodiments, the second sample is an adipose sample. In some embodiments, the HGFAC mRNA measurement is reduced by about 2.5% or more, about 5% or more, or about 7.5% or more, relative to the baseline HGFAC mRNA measurement. In some embodiments, the HGFAC mRNA measurement is decreased by about 10% or more, relative to the baseline HGFAC mRNA measurement. In some embodiments, the HGFAC mRNA measurement is decreased by about 20% or more, about 30% or more, about 40% or more, about 50% or more, about 60% or more, about 70% or more, about 80% or more, about 90% or more, or about 100%, relative to the baseline HGFAC mRNA measurement. In some embodiments, the HGFAC mRNA measurement is decreased by no more than about 2.5%, no more than about 5%, or no more than about 7.5%, relative to the baseline HGFAC mRNA measurement. In some embodiments, the HGFAC mRNA measurement is decreased by no more than about 10%, relative to the baseline HGFAC mRNA measurement. In some embodiments, the HGFAC mRNA measurement is decreased by no more than about 20%, no more than about 30%, no more than about 40%, no more than about 50%, no more than about 60%, no more than about 70%, no more than about 80%, no more than about 90%, or no more than about 100%, relative to the baseline HGFAC mRNA measurement. In some embodiments, the HGFAC mRNA measurement is decreased by 2.5%, 5%, 7.5%, 10%, 20%, 30%, 40%, 50%, 60%, 70%, 80%, 90%, or 100% or by a range defined by any of the two aforementioned percentages.

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments. In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may include DNA. For example, the oligonucleotide may include 2′ deoxyribonucleotides. An ASO may comprise or consist of DNA.

The term “Cx-y” or “Cx-Cy” when used in conjunction with a chemical moiety, such as alkyl, alkenyl, or alkynyl is meant to include groups that contain from x to y carbons in the chain. For example, the term “C1-6alkyl” refers to substituted or unsubstituted saturated hydrocarbon groups, including straight-chain alkyl and branched-chain alkyl groups that contain from 1 to 6 carbons.

The terms “Cx-yalkenyl” and “Cx-yalkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings.

In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms.

The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2 trifluoroethyl, 1 chloromethyl 2 fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms.

Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3 benzodioxolyl, benzofuranyl, benzoxazolyl, benzo[d]thiazolyl, benzothiadiazolyl, benzo[b][1,4]dioxepinyl, benzo[b][1,4]oxazinyl, 1,4 benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2 d]pyrimidinyl, benzotriazolyl, benzo[4,6]imidazo[1,2 a]pyridinyl, carbazolyl, cinnolinyl, cyclopenta[d]pyrimidinyl, 6,7 dihydro 5H cyclopenta[4,5]thieno[2,3 d]pyrimidinyl, 5,6 dihydrobenzo[h]quinazolinyl, 5,6 dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2 c]pyridinyl, 5,6,7,8,9,10 hexahydrocycloocta[d]pyrimidinyl, 5,6,7,8,9,10 hexahydrocycloocta[d]pyridazinyl, 5,6,7,8,9,10 hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8 methano 5,6,7,8 tetrahydroquinazolinyl, naphthyridinyl, 1,6 naphthyridinonyl, oxadiazolyl, 2 oxoazepinyl, oxazolyl, oxiranyl, 5,6,6a,7,8,9,10,10a octahydrobenzo[h]quinazolinyl, 1 phenyl 1H pyrrolyl, phenazinyl, phenothiazinyl, phenoxazinyl, phthalazinyl, pteridinyl, purinyl, pyrrolyl, pyrazolyl, pyrazolo[3,4 d]pyrimidinyl, pyridinyl, pyrido[3,2 d]pyrimidinyl, pyrido[3,4 d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8 tetrahydroquinazolinyl, 5,6,7,8 tetrahydrobenzo[4,5]thieno[2,3 d]pyrimidinyl, 6,7,8,9 tetrahydro 5H cyclohepta[4,5]thieno[2,3 d]pyrimidinyl, 5,6,7,8 tetrahydropyrido[4,5 c]pyridazinyl, thiazolyl, thiadiazolyl, triazolyl, tetrazolyl, triazinyl, thieno[2,3 d]pyrimidinyl, thieno[3,2 d]pyrimidinyl, thieno[2,3 c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2 oxopiperazinyl, 2 oxopiperidinyl, 2 oxopyrrolidinyl, oxazolidinyl, piperidinyl, piperazinyl, 4 piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1 oxo thiomorpholinyl, 2-oxa-6-azaspiro[3.3]heptane, and 1,1 dioxo thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH2 of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH2), —Rb ORa, —Rb OC(O) Ra, —Rb OC(O) ORa, —Rb OC(O) N(Ra)₂, —Rb N(Ra)₂, —Rb C(O)Ra, —Rb C(O)ORa, —Rb C(O)N(Ra)₂, —Rb O Re C(O)N(Ra)₂, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2), and —Rb S(O)tN(Ra)₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH2), —Rb ORa, —Rb OC(O) Ra, —Rb OC(O) ORa, —Rb OC(O) N(Ra)₂, —Rb N(Ra)₂, —Rb C(O)Ra, —Rb C(O)ORa, —Rb C(O)N(Ra)₂, —Rb O Re C(O)N(Ra)₂, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)₂ (where t is 1 or 2); wherein each Ra is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each Ra, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH2), —Rb ORa, —Rb OC(O) Ra, —Rb OC(O) ORa, —Rb OC(O) N(Ra)₂, —Rb N(Ra)₂, —Rb C(O)Ra, —Rb C(O)ORa, —Rb C(O)N(Ra)₂, —Rb O Re C(O)N(Ra)₂, —Rb N(Ra)C(O)ORa, —Rb N(Ra)C(O)Ra, —Rb N(Ra)S(O)tRa (where t is 1 or 2), —Rb S(O)tRa (where t is 1 or 2), —Rb S(O)tORa (where t is 1 or 2) and —Rb S(O)tN(Ra)₂ (where t is 1 or 2); and wherein each Rb is independently selected from a direct bond or a straight or branched alkylene, alkenylene, or alkynylene chain, and each Re is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

Some embodiments refer to nucleic acid sequence information. It is contemplated that in some embodiments, thymine (T) may be interchanged with uracil (U), or vice versa. For example, some sequences in the sequence listing may recite Ts, but these may be replaced with Us in some embodiments.

In some oligonucleotides with nucleic acid sequences that include uracil, the uracil may be replaced with thymine. Similarly, in some oligonucleotides with nucleic acid sequences that include thymine, the thymine may be replaced with uracil. In some embodiments, an oligonucleotide such as an siRNA comprises or consists of RNA. In some embodiments, the oligonucleotide may include DNA. For example, the oligonucleotide may include 2′ deoxyribonucleotides. An ASO may comprise or consist of DNA. To any extent that the sequence listing contradicts the disclosure in the specification, the specification takes precedent.

Some aspects include sequences with nucleotide modifications or modified internucleoside linkages. Generally, and unless otherwise specified, Nf (e.g. Af, Cf, Gf, Tf, or Uf) refers to a 2′ fluoro-modified nucleoside, dN (e.g. dA, dC, dG, dT, or dU) refers to a 2′ deoxy nucleoside, n (e.g. a, c, g, t, or u) refers to a 2′ O-methyl modified nucleoside, and “s” refers to a phosphorothioate linkage.

A pyrimidine may include cytosine (C), thymine (T), or uracil (U). A pyrimidine may include C or U. A pyrimidine may include C or T. Where a pyrimidine is referred to, it may indicate a nucleoside or nucleotide comprising a pyrimidine. A purine may include guanine (G) or adenine (A). Where a purine is referred to, it may indicate a nucleoside or nucleotide comprising a purine.

The section headings used herein are for organizational purposes only and are not to be construed as limiting the subject matter described.

### VI. EXAMPLES

### Example 1: Rare, Predicted-Deleterious Variants in the HGFAC Gene Demonstrate Inverse Associations for Malignancies Vs. Autoimmune Diseases

HGFAC variants were evaluated for associations with a variety of cancer and immunological traits in approximately 452,000 individuals with genotype data from the UK Biobank cohort. Variants were evaluated in a gene burden test comprised of a total of 22 rare, predicted-deleterious coding variants: 15 variants annotated as deleterious missense, 4 annotated frameshift variants, 2 annotated splice donor variants and 1 annotated stop gain variant. Table 2 lists these variants. It was hypothesized that individually these variants would result in a decrease in the abundance and activity of the HGFAC gene product, and that it is this loss of function that would lead to the observed genetic associations.

TABLE 2
HGFAC gene variants included in the gene burden test

| Variant | rsID | Consequence |
|---|---|---|
| chr4:3444054:G:C | rs564042558 | deleterious_missense_variant |
| chr4:3444143:G:C | rs917199559 | deleterious_missense_variant |
| chr4:3444395:G:A | rs748413339 | deleterious_missense_variant |
| chr4:3444423:C:G | rs201082880 | deleterious_missense_variant |
| chr4:3444433:C:T | rs780551152 | stop_gained |
| chr4:3444975:G:A | rs545303850 | deleterious_missense_variant |
| chr4:3444992:C:G | rs373478970 | deleterious_missense_variant |
| chr4:3444992:C:T | rs373478970 | deleterious_missense_variant |
| chr4:3444993:G:A | rs1365995245 | deleterious_missense_variant |
| chr4:3446120:G:T | rs1010904877 | deleterious_missense_variant |
| chr4:3446239:G:A | rs369079003 | deleterious_missense_variant |
| chr4:3447576:G:GA | rs765340154 | frameshift_variant |
| chr4:3447625:G:A | rs996743028 | deleterious_missense_variant |
| chr4:3447631:G:GGTGA | rs758675316 | frameshift_variant |
| chr4:3447633:T:C | rs139240360 | splice_donor_variant |
| chr4:3447965:GC:G | rs907650708 | frameshift_variant |
| chr4:3448167:TC:T | rs760119184 | frameshift_variant |
| chr4:3448236:T:G | rs138538142 | deleterious_missense_variant |
| chr4:3448277:G:A | rs757531620 | splice_donor_variant |
| chr4:3449291:G:A | rs769181942 | deleterious_missense_variant |
| chr4:3449318:G:A | rs375925462 | deleterious_missense_variant |
| chr4:3449351:G:A | rs559231140 | deleterious_missense_variant |

The analyses resulted in identification of associations with the HGFAC burden test and several cancer and autoimmune disease traits (Table 3). For example, there were protective associations with cancer in a pan-cancer case-control study. The HGFAC burden test was associated with protection from individual cancers, including malignant neoplasms of the digestive organs. Additionally, the HGFAC burden test was associated with increased risk of autoimmune diseases, including specified forms of hypothyroidism and systemic sclerosis (scleroderma).

TABLE 3
Associations with cancer and autoimmune disease traits

| | | | Malignant | | Malignant neoplasms | | Specified | | Systemic sclerosis | |
|---|---|---|---|---|---|---|---|---|---|---|
| | | | Neoplasm | | of digestive organs | | Hypothyroidism | | [scleroderma] | |
| | | | (n = 89350) | | (n = 15497) | | (n = 2599) | | (n = 802) | |
| | | | P | | P | | P | | P | |
| Test | Gene | AAF | value | OR | value | OR | value | OR | value | OR |
| Burden | HGFAC | 0.0009 | 5.77E−05 | ↓0.683 | 0.003 | ↓0.480 | 2.24E−05 | ↑3.402 | 2.35E−04 | ↑5.114 |

The results indicated that reduced abundance/activity of HGFAC resulted in protection from malignant neoplasms and increased risk of autoimmune disease; and suggested that therapeutic inhibition of HGFAC may represent a novel approach to immunotherapy that may be used in the treatment of a variety of cancers.

### Example 2: Bioinformatic Selection of Sequences in Order to Identify Therapeutic siRNAs to Down Modulate Expression of the HGFAC mRNA

Screening sets were defined based on bioinformatic analysis. Therapeutic siRNAs were designed to target human HGFAC. Predicted specificity in human, rhesus monkey, cynomolgus monkey, mouse, rat, rabbit, dog, gerbil, syrian hamster, chinese hamster, guinea pig and naked mole rat was determined for sense (S) and antisense (AS) strands. These were assigned a “specificity score” which considers the likelihood of unintended downregulation of any other transcript by full or partial complementarity of an siRNA strand (up to 2 mismatches within positions 2-18) as well as the number and positions of mismatches. Thus, off-target(s) transcripts for antisense and sense strands of each siRNA were identified. As identified, siRNAs with high specificity and a low number of predicted off-targets provided a benefit of increased targeting specificity.

In addition to selecting siRNA sequences with high sequence specificity to HGFAC mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions can be avoided. Seed regions identified in miRNAs from human, mouse, rat, rhesus monkey, dog, rabbit, and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Analysis of the Genome Aggregation Database (gnomAD) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant HGFAC mRNA sequence revealed few sequences that fulfil the specificity parameters and at the same time target HGFAC mRNA in all the analyzed relevant species. Therefore, independent screening subsets were designed for the therapeutic siRNAs.

The siRNAs in these subsets recognized at least the human HGFAC sequences. Therefore, the siRNAs in these subsets can be used to target human HGFAC in a therapeutic setting.

The number of siRNA sequences derived from human HGFAC mRNA (ENST00000382774, SEQ ID NO: 4193) without consideration of specificity or species cross-reactivity was 2051 (sense and antisense strand sequences included in SEQ ID NOS: 1-2051 and 2052-4102, respectively)

Prioritizing sequences for target specificity, miRNA seed region sequences and SNPs as described above yields subset A. Subset A contains 380 siRNAs whose base sequences are shown in Table A.

TABLE A
Subset A

| | SEQ ID | sense strand | SEQ ID | antisense strand |
|---|---|---|---|---|
| siRNA NO: | NO: | sequence (5′-3′) | NO: | sequence (5′-3′) |
| siRNA 26 | 26 | UCAGGAGCCAUGGGGCGCU | 2077 | AGCGCCCCAUGGCUCCUGA |
| siRNA 114 | 114 | UGCUGCUGCCACGGGGGUU | 2165 | AACCCCCGUGGCAGCAGCA |
| siRNA 116 | 116 | CUGCUGCCACGGGGGUUCC | 2167 | GGAACCCCCGUGGCAGCAG |
| siRNA 118 | 118 | GCUGCCACGGGGGUUCCAG | 2169 | CUGGAACCCCCGUGGCAGC |
| siRNA 122 | 122 | CCACGGGGGUUCCAGCCCC | 2173 | GGGGCUGGAACCCCCGUGG |
| siRNA 136 | 136 | GCCCCAGCCUGGCGGGAAC | 2187 | GUUCCCGCCAGGCUGGGGC |
| siRNA 137 | 137 | CCCCAGCCUGGCGGGAACC | 2188 | GGUUCCCGCCAGGCUGGGG |
| siRNA 140 | 140 | CAGCCUGGCGGGAACCGUA | 2191 | UACGGUUCCCGCCAGGCUG |
| siRNA 141 | 141 | AGCCUGGCGGGAACCGUAC | 2192 | GUACGGUUCCCGCCAGGCU |
| siRNA 143 | 143 | CCUGGCGGGAACCGUACGG | 2194 | CCGUACGGUUCCCGCCAGG |
| siRNA 144 | 144 | CUGGCGGGAACCGUACGGA | 2195 | UCCGUACGGUUCCCGCCAG |
| siRNA 145 | 145 | UGGCGGGAACCGUACGGAG | 2196 | CUCCGUACGGUUCCCGCCA |
| siRNA 146 | 146 | GGCGGGAACCGUACGGAGU | 2197 | ACUCCGUACGGUUCCCGCC |
| siRNA 149 | 149 | GGGAACCGUACGGAGUCCC | 2200 | GGGACUCCGUACGGUUCCC |
| siRNA 150 | 150 | GGAACCGUACGGAGUCCCC | 2201 | GGGGACUCCGUACGGUUCC |
| siRNA 152 | 152 | AACCGUACGGAGUCCCCAG | 2203 | CUGGGGACUCCGUACGGUU |
| siRNA 154 | 154 | CCGUACGGAGUCCCCAGAA | 2205 | UUCUGGGGACUCCGUACGG |
| siRNA 155 | 155 | CGUACGGAGUCCCCAGAAC | 2206 | GUUCUGGGGACUCCGUACG |
| siRNA 158 | 158 | ACGGAGUCCCCAGAACCUA | 2209 | UAGGUUCUGGGGACUCCGU |
| siRNA 160 | 160 | GGAGUCCCCAGAACCUAAU | 2211 | AUUAGGUUCUGGGGACUCC |
| siRNA 165 | 165 | CCCCAGAACCUAAUGCCAC | 2216 | GUGGCAUUAGGUUCUGGGG |
| siRNA 166 | 166 | CCCAGAACCUAAUGCCACA | 2217 | UGUGGCAUUAGGUUCUGGG |
| siRNA 167 | 167 | CCAGAACCUAAUGCCACAG | 2218 | CUGUGGCAUUAGGUUCUGG |
| siRNA 172 | 172 | ACCUAAUGCCACAGCGACC | 2223 | GGUCGCUGUGGCAUUAGGU |
| siRNA 173 | 173 | CCUAAUGCCACAGCGACCC | 2224 | GGGUCGCUGUGGCAUUAGG |
| siRNA 199 | 199 | CCCCACUAUCCUGGUGACC | 2250 | GGUCACCAGGAUAGUGGGG |
| siRNA 202 | 202 | CACUAUCCUGGUGACCUCU | 2253 | AGAGGUCACCAGGAUAGUG |
| siRNA 203 | 203 | ACUAUCCUGGUGACCUCUG | 2254 | CAGAGGUCACCAGGAUAGU |
| siRNA 205 | 205 | UAUCCUGGUGACCUCUGUG | 2256 | CACAGAGGUCACCAGGAUA |
| siRNA 232 | 232 | GACCCCAGCAACAAGUGCU | 2283 | AGCACUUGUUGCUGGGGUC |
| siRNA 294 | 294 | CCAGGGCAGUUCCCUCGAG | 2345 | CUCGAGGGAACUGCCCUGG |
| siRNA 295 | 295 | CAGGGCAGUUCCCUCGAGC | 2346 | GCUCGAGGGAACUGCCCUG |
| siRNA 296 | 296 | AGGGCAGUUCCCUCGAGCA | 2347 | UGCUCGAGGGAACUGCCCU |
| siRNA 301 | 301 | AGUUCCCUCGAGCAGUAGC | 2352 | GCUACUGCUCGAGGGAACU |
| siRNA 303 | 303 | UUCCCUCGAGCAGUAGCCC | 2354 | GGGCUACUGCUCGAGGGAA |
| siRNA 304 | 304 | UCCCUCGAGCAGUAGCCCC | 2355 | GGGGCUACUGCUCGAGGGA |
| siRNA 305 | 305 | CCCUCGAGCAGUAGCCCCC | 2356 | GGGGGCUACUGCUCGAGGG |
| siRNA 315 | 315 | GUAGCCCCCAGGCCCAAGC | 2366 | GCUUGGGCCUGGGGGCUAC |
| siRNA 328 | 328 | CCAAGCACUCACCGAGGAC | 2379 | GUCCUCGGUGAGUGCUUGG |
| siRNA 334 | 334 | ACUCACCGAGGACGGGAGG | 2385 | CCUCCCGUCCUCGGUGAGU |
| siRNA 335 | 335 | CUCACCGAGGACGGGAGGC | 2386 | GCCUCCCGUCCUCGGUGAG |
| siRNA 336 | 336 | UCACCGAGGACGGGAGGCC | 2387 | GGCCUCCCGUCCUCGGUGA |
| siRNA 360 | 360 | GGUUCCCCUUCCGCUACGG | 2411 | CCGUAGCGGAAGGGGAACC |
| siRNA 361 | 361 | GUUCCCCUUCCGCUACGGG | 2412 | CCCGUAGCGGAAGGGGAAC |
| siRNA 362 | 362 | UUCCCCUUCCGCUACGGGG | 2413 | CCCCGUAGCGGAAGGGGAA |
| siRNA 363 | 363 | UCCCCUUCCGCUACGGGGG | 2414 | CCCCCGUAGCGGAAGGGGA |
| siRNA 364 | 364 | CCCCUUCCGCUACGGGGGC | 2415 | GCCCCCGUAGCGGAAGGGG |
| siRNA 367 | 367 | CUUCCGCUACGGGGGCCGC | 2418 | GCGGCCCCCGUAGCGGAAG |
| siRNA 371 | 371 | CGCUACGGGGGCCGCAUGC | 2422 | GCAUGCGGCCCCCGUAGCG |
| siRNA 372 | 372 | GCUACGGGGGCCGCAUGCU | 2423 | AGCAUGCGGCCCCCGUAGC |
| siRNA 373 | 373 | CUACGGGGGCCGCAUGCUG | 2424 | CAGCAUGCGGCCCCCGUAG |
| SiRNA 374 | 374 | UACGGGGGCCGCAUGCUGC | 2425 | GCAGCAUGCGGCCCCCGUA |
| siRNA 378 | 378 | GGGGCCGCAUGCUGCAUGC | 2429 | GCAUGCAGCAUGCGGCCCC |
| siRNA 389 | 389 | CUGCAUGCCUGCACUUCGG | 2440 | CCGAAGUGCAGGCAUGCAG |
| siRNA 390 | 390 | UGCAUGCCUGCACUUCGGA | 2441 | UCCGAAGUGCAGGCAUGCA |
| siRNA 391 | 391 | GCAUGCCUGCACUUCGGAG | 2442 | CUCCGAAGUGCAGGCAUGC |
| siRNA 395 | 395 | GCCUGCACUUCGGAGGGCA | 2446 | UGCCCUCCGAAGUGCAGGC |
| siRNA 398 | 398 | UGCACUUCGGAGGGCAGUG | 2449 | CACUGCCCUCCGAAGUGCA |
| siRNA 431 | 431 | UGUGCCACAACUCACAACU | 2482 | AGUUGUGAGUUGUGGCACA |
| siRNA 434 | 434 | GCCACAACUCACAACUACG | 2485 | CGUAGUUGUGAGUUGUGGC |
| siRNA 436 | 436 | CACAACUCACAACUACGAC | 2487 | GUCGUAGUUGUGAGUUGUG |
| siRNA 437 | 437 | ACAACUCACAACUACGACC | 2488 | GGUCGUAGUUGUGAGUUGU |
| siRNA 438 | 438 | CAACUCACAACUACGACCG | 2489 | CGGUCGUAGUUGUGAGUUG |
| siRNA 442 | 442 | UCACAACUACGACCGGGAC | 2493 | GUCCCGGUCGUAGUUGUGA |
| siRNA 443 | 443 | CACAACUACGACCGGGACA | 2494 | UGUCCCGGUCGUAGUUGUG |
| siRNA 445 | 445 | CAACUACGACCGGGACAGG | 2496 | CCUGUCCCGGUCGUAGUUG |
| siRNA 446 | 446 | AACUACGACCGGGACAGGG | 2497 | CCCUGUCCCGGUCGUAGUU |
| siRNA 469 | 469 | GGGCUACUGUGUGGAGGCC | 2520 | GGCCUCCACACAGUAGCCC |
| siRNA 472 | 472 | CUACUGUGUGGAGGCCACC | 2523 | GGUGGCCUCCACACAGUAG |
| siRNA 473 | 473 | UACUGUGUGGAGGCCACCC | 2524 | GGGUGGCCUCCACACAGUA |
| siRNA 530 | 530 | UCCGGCCCCUGCCUCAAUG | 2581 | CAUUGAGGCAGGGGCCGGA |
| siRNA 531 | 531 | CCGGCCCCUGCCUCAAUGG | 2582 | CCAUUGAGGCAGGGGCCGG |
| siRNA 533 | 533 | GGCCCCUGCCUCAAUGGAG | 2584 | CUCCAUUGAGGCAGGGGCC |
| siRNA 549 | 549 | GAGGCUCCUGCUCCAAUAC | 2600 | GUAUUGGAGCAGGAGCCUC |
| siRNA 558 | 558 | GCUCCAAUACCCAGGACCC | 2609 | GGGUCCUGGGUAUUGGAGC |
| siRNA 562 | 562 | CAAUACCCAGGACCCCCAG | 2613 | CUGGGGGUCCUGGGUAUUG |
| siRNA 564 | 564 | AUACCCAGGACCCCCAGUC | 2615 | GACUGGGGGUCCUGGGUAU |
| siRNA 569 | 569 | CAGGACCCCCAGUCCUAUC | 2620 | GAUAGGACUGGGGGUCCUG |
| siRNA 575 | 575 | CCCCAGUCCUAUCACUGCA | 2626 | UGCAGUGAUAGGACUGGGG |
| siRNA 601 | 601 | CCGGGCCUUCACCGGCAAG | 2652 | CUUGCCGGUGAAGGCCCGG |
| siRNA 602 | 602 | CGGGCCUUCACCGGCAAGG | 2653 | CCUUGCCGGUGAAGGCCCG |
| siRNA 603 | 603 | GGGCCUUCACCGGCAAGGA | 2654 | UCCUUGCCGGUGAAGGCCC |
| siRNA 604 | 604 | GGCCUUCACCGGCAAGGAC | 2655 | GUCCUUGCCGGUGAAGGCC |
| siRNA 610 | 610 | CACCGGCAAGGACUGCGGC | 2661 | GCCGCAGUCCUUGCCGGUG |
| siRNA 612 | 612 | CCGGCAAGGACUGCGGCAC | 2663 | GUGCCGCAGUCCUUGCCGG |
| siRNA 616 | 616 | CAAGGACUGCGGCACAGAG | 2667 | CUCUGUGCCGCAGUCCUUG |
| siRNA 624 | 624 | GCGGCACAGAGAAAUGCUU | 2675 | AAGCAUUUCUCUGUGCCGC |
| siRNA 628 | 628 | CACAGAGAAAUGCUUUGAU | 2679 | AUCAAAGCAUUUCUCUGUG |
| siRNA 729 | 729 | GGGGCCGGACCUGGUGCGA | 2780 | UCGCACCAGGUCCGGCCCC |
| siRNA 730 | 730 | GGGCCGGACCUGGUGCGAA | 2781 | UUCGCACCAGGUCCGGCCC |
| siRNA 732 | 732 | GCCGGACCUGGUGCGAAGG | 2783 | CCUUCGCACCAGGUCCGGC |
| siRNA 733 | 733 | CCGGACCUGGUGCGAAGGC | 2784 | GCCUUCGCACCAGGUCCGG |
| siRNA 735 | 735 | GGACCUGGUGCGAAGGCAC | 2786 | GUGCCUUCGCACCAGGUCC |
| siRNA 736 | 736 | GACCUGGUGCGAAGGCACC | 2787 | GGUGCCUUCGCACCAGGUC |
| siRNA 737 | 737 | ACCUGGUGCGAAGGCACCC | 2788 | GGGUGCCUUCGCACCAGGU |
| siRNA 738 | 738 | CCUGGUGCGAAGGCACCCG | 2789 | CGGGUGCCUUCGCACCAGG |
| siRNA 742 | 742 | GUGCGAAGGCACCCGACAU | 2793 | AUGUCGGGUGCCUUCGCAC |
| siRNA 743 | 743 | UGCGAAGGCACCCGACAUA | 2794 | UAUGUCGGGUGCCUUCGCA |
| siRNA 744 | 744 | GCGAAGGCACCCGACAUAC | 2795 | GUAUGUCGGGUGCCUUCGC |
| siRNA 745 | 745 | CGAAGGCACCCGACAUACA | 2796 | UGUAUGUCGGGUGCCUUCG |
| siRNA 747 | 747 | AAGGCACCCGACAUACAGC | 2798 | GCUGUAUGUCGGGUGCCUU |
| siRNA 748 | 748 | AGGCACCCGACAUACAGCU | 2799 | AGCUGUAUGUCGGGUGCCU |
| siRNA 749 | 749 | GGCACCCGACAUACAGCUU | 2800 | AAGCUGUAUGUCGGGUGCC |
| siRNA 751 | 751 | CACCCGACAUACAGCUUGU | 2802 | ACAAGCUGUAUGUCGGGUG |
| siRNA 753 | 753 | CCCGACAUACAGCUUGUCU | 2804 | AGACAAGCUGUAUGUCGGG |
| siRNA 754 | 754 | CCGACAUACAGCUUGUCUG | 2805 | CAGACAAGCUGUAUGUCGG |
| siRNA 755 | 755 | CGACAUACAGCUUGUCUGA | 2806 | UCAGACAAGCUGUAUGUCG |
| siRNA 756 | 756 | GACAUACAGCUUGUCUGAG | 2807 | CUCAGACAAGCUGUAUGUC |
| siRNA 757 | 757 | ACAUACAGCUUGUCUGAGC | 2808 | GCUCAGACAAGCUGUAUGU |
| siRNA 758 | 758 | CAUACAGCUUGUCUGAGCA | 2809 | UGCUCAGACAAGCUGUAUG |
| siRNA 776 | 776 | AGCCCUUGCCUGAACGGGG | 2827 | CCCCGUUCAGGCAAGGGCU |
| siRNA 796 | 796 | CACCUGCCACCUGAUCGUG | 2847 | CACGAUCAGGUGGCAGGUG |
| siRNA 799 | 799 | CUGCCACCUGAUCGUGGCC | 2850 | GGCCACGAUCAGGUGGCAG |
| siRNA 810 | 810 | UCGUGGCCACCGGGACCAC | 2861 | GUGGUCCCGGUGGCCACGA |
| siRNA 819 | 819 | CCGGGACCACCGUGUGUGC | 2870 | GCACACACGGUGGUCCCGG |
| siRNA 848 | 848 | GGCUUCGCUGGACGGCUCU | 2899 | AGAGCCGUCCAGCGAAGCC |
| siRNA 853 | 853 | CGCUGGACGGCUCUGCAAC | 2904 | GUUGCAGAGCCGUCCAGCG |
| siRNA 855 | 855 | CUGGACGGCUCUGCAACAU | 2906 | AUGUUGCAGAGCCGUCCAG |
| siRNA 857 | 857 | GGACGGCUCUGCAACAUCG | 2908 | CGAUGUUGCAGAGCCGUCC |
| siRNA 858 | 858 | GACGGCUCUGCAACAUCGA | 2909 | UCGAUGUUGCAGAGCCGUC |
| siRNA 859 | 859 | ACGGCUCUGCAACAUCGAG | 2910 | CUCGAUGUUGCAGAGCCGU |
| siRNA 861 | 861 | GGCUCUGCAACAUCGAGCC | 2912 | GGCUCGAUGUUGCAGAGCC |
| siRNA 862 | 862 | GCUCUGCAACAUCGAGCCU | 2913 | AGGCUCGAUGUUGCAGAGC |
| siRNA 863 | 863 | CUCUGCAACAUCGAGCCUG | 2914 | CAGGCUCGAUGUUGCAGAG |
| siRNA 871 | 871 | CAUCGAGCCUGAUGAGCGC | 2922 | GCGCUCAUCAGGCUCGAUG |
| siRNA 872 | 872 | AUCGAGCCUGAUGAGCGCU | 2923 | AGCGCUCAUCAGGCUCGAU |
| siRNA 873 | 873 | UCGAGCCUGAUGAGCGCUG | 2924 | CAGCGCUCAUCAGGCUCGA |
| siRNA 875 | 875 | GAGCCUGAUGAGCGCUGCU | 2926 | AGCAGCGCUCAUCAGGCUC |
| siRNA 879 | 879 | CUGAUGAGCGCUGCUUCUU | 2930 | AAGAAGCAGCGCUCAUCAG |
| siRNA 896 | 896 | UUGGGGAACGGCACUGGGU | 2947 | ACCCAGUGCCGUUCCCCAA |
| siRNA 901 | 901 | GAACGGCACUGGGUACCGU | 2952 | ACGGUACCCAGUGCCGUUC |
| siRNA 902 | 902 | AACGGCACUGGGUACCGUG | 2953 | CACGGUACCCAGUGCCGUU |
| siRNA 908 | 908 | ACUGGGUACCGUGGCGUGG | 2959 | CCACGCCACGGUACCCAGU |
| siRNA 909 | 909 | CUGGGUACCGUGGCGUGGC | 2960 | GCCACGCCACGGUACCCAG |
| siRNA 910 | 910 | UGGGUACCGUGGCGUGGCC | 2961 | GGCCACGCCACGGUACCCA |
| siRNA 911 | 911 | GGGUACCGUGGCGUGGCCA | 2962 | UGGCCACGCCACGGUACCC |
| siRNA 912 | 912 | GGUACCGUGGCGUGGCCAG | 2963 | CUGGCCACGCCACGGUACC |
| siRNA 913 | 913 | GUACCGUGGCGUGGCCAGC | 2964 | GCUGGCCACGCCACGGUAC |
| siRNA 916 | 916 | CCGUGGCGUGGCCAGCACC | 2967 | GGUGCUGGCCACGCCACGG |
| siRNA 957 | 957 | UGGCCUGGAACUCCGAUCU | 3008 | AGAUCGGAGUUCCAGGCCA |
| SiRNA 959 | 959 | GCCUGGAACUCCGAUCUGC | 3010 | GCAGAUCGGAGUUCCAGGC |
| siRNA 960 | 960 | CCUGGAACUCCGAUCUGCU | 3011 | AGCAGAUCGGAGUUCCAGG |
| siRNA 961 | 961 | CUGGAACUCCGAUCUGCUC | 3012 | GAGCAGAUCGGAGUUCCAG |
| siRNA 962 | 962 | UGGAACUCCGAUCUGCUCU | 3013 | AGAGCAGAUCGGAGUUCCA |
| siRNA 964 | 964 | GAACUCCGAUCUGCUCUAC | 3015 | GUAGAGCAGAUCGGAGUUC |
| siRNA 965 | 965 | AACUCCGAUCUGCUCUACC | 3016 | GGUAGAGCAGAUCGGAGUU |
| siRNA 992 | 992 | CACGUGGACUCCGUGGGCG | 3043 | CGCCCACGGAGUCCACGUG |
| siRNA 996 | 996 | UGGACUCCGUGGGCGCCGC | 3047 | GCGGCGCCCACGGAGUCCA |
| siRNA 998 | 998 | GACUCCGUGGGCGCCGCGG | 3049 | CCGCGGCGCCCACGGAGUC |
| siRNA 1027 | 1027 | CCUGGGCCCCCAUGCCUAC | 3078 | GUAGGCAUGGGGGCCCAGG |
| siRNA 1028 | 1028 | CUGGGCCCCCAUGCCUACU | 3079 | AGUAGGCAUGGGGGCCCAG |
| siRNA 1033 | 1033 | CCCCCAUGCCUACUGCCGG | 3084 | CCGGCAGUAGGCAUGGGGG |
| siRNA 1034 | 1034 | CCCCAUGCCUACUGCCGGA | 3085 | UCCGGCAGUAGGCAUGGGG |
| siRNA 1035 | 1035 | CCCAUGCCUACUGCCGGAA | 3086 | UUCCGGCAGUAGGCAUGGG |
| siRNA 1039 | 1039 | UGCCUACUGCCGGAAUCCG | 3090 | CGGAUUCCGGCAGUAGGCA |
| siRNA 1040 | 1040 | GCCUACUGCCGGAAUCCGG | 3091 | CCGGAUUCCGGCAGUAGGC |
| siRNA 1041 | 1041 | CCUACUGCCGGAAUCCGGA | 3092 | UCCGGAUUCCGGCAGUAGG |
| siRNA 1042 | 1042 | CUACUGCCGGAAUCCGGAC | 3093 | GUCCGGAUUCCGGCAGUAG |
| siRNA 1043 | 1043 | UACUGCCGGAAUCCGGACA | 3094 | UGUCCGGAUUCCGGCAGUA |
| siRNA 1047 | 1047 | GCCGGAAUCCGGACAAUGA | 3098 | UCAUUGUCCGGAUUCCGGC |
| siRNA 1048 | 1048 | CCGGAAUCCGGACAAUGAC | 3099 | GUCAUUGUCCGGAUUCCGG |
| siRNA 1050 | 1050 | GGAAUCCGGACAAUGACGA | 3101 | UCGUCAUUGUCCGGAUUCC |
| siRNA 1051 | 1051 | GAAUCCGGACAAUGACGAG | 3102 | CUCGUCAUUGUCCGGAUUC |
| siRNA 1052 | 1052 | AAUCCGGACAAUGACGAGA | 3103 | UCUCGUCAUUGUCCGGAUU |
| siRNA 1053 | 1053 | AUCCGGACAAUGACGAGAG | 3104 | CUCUCGUCAUUGUCCGGAU |
| siRNA 1054 | 1054 | UCCGGACAAUGACGAGAGG | 3105 | CCUCUCGUCAUUGUCCGGA |
| siRNA 1055 | 1055 | CCGGACAAUGACGAGAGGC | 3106 | GCCUCUCGUCAUUGUCCGG |
| siRNA 1067 | 1067 | GAGAGGCCCUGGUGCUACG | 3118 | CGUAGCACCAGGGCCUCUC |
| siRNA 1100 | 1100 | GCGCUCUCCUGGGAGUACU | 3151 | AGUACUCCCAGGAGAGCGC |
| siRNA 1106 | 1106 | UCCUGGGAGUACUGCCGCC | 3157 | GGCGGCAGUACUCCCAGGA |
| siRNA 1121 | 1121 | CGCCUGGAGGCCUGCGAAU | 3172 | AUUCGCAGGCCUCCAGGCG |
| siRNA 1122 | 1122 | GCCUGGAGGCCUGCGAAUC | 3173 | GAUUCGCAGGCCUCCAGGC |
| siRNA 1123 | 1123 | CCUGGAGGCCUGCGAAUCC | 3174 | GGAUUCGCAGGCCUCCAGG |
| siRNA 1131 | 1131 | CCUGCGAAUCCCUCACCAG | 3182 | CUGGUGAGGGAUUCGCAGG |
| siRNA 1133 | 1133 | UGCGAAUCCCUCACCAGAG | 3184 | CUCUGGUGAGGGAUUCGCA |
| siRNA 1136 | 1136 | GAAUCCCUCACCAGAGUCC | 3187 | GGACUCUGGUGAGGGAUUC |
| siRNA 1141 | 1141 | CCUCACCAGAGUCCAACUG | 3192 | CAGUUGGACUCUGGUGAGG |
| siRNA 1143 | 1143 | UCACCAGAGUCCAACUGUC | 3194 | GACAGUUGGACUCUGGUGA |
| siRNA 1144 | 1144 | CACCAGAGUCCAACUGUCA | 3195 | UGACAGUUGGACUCUGGUG |
| siRNA 1149 | 1149 | GAGUCCAACUGUCACCGGA | 3200 | UCCGGUGACAGUUGGACUC |
| siRNA 1151 | 1151 | GUCCAACUGUCACCGGAUC | 3202 | GAUCCGGUGACAGUUGGAC |
| siRNA 1152 | 1152 | UCCAACUGUCACCGGAUCU | 3203 | AGAUCCGGUGACAGUUGGA |
| siRNA 1153 | 1153 | CCAACUGUCACCGGAUCUC | 3204 | GAGAUCCGGUGACAGUUGG |
| siRNA 1161 | 1161 | CACCGGAUCUCCUGGCGAC | 3212 | GUCGCCAGGAGAUCCGGUG |
| siRNA 1166 | 1166 | GAUCUCCUGGCGACCCUGC | 3217 | GCAGGGUCGCCAGGAGAUC |
| siRNA 1168 | 1168 | UCUCCUGGCGACCCUGCCU | 3219 | AGGCAGGGUCGCCAGGAGA |
| siRNA 1203 | 1203 | GGCGCCAGGCCUGCGGCAG | 3254 | CUGCCGCAGGCCUGGCGCC |
| siRNA 1215 | 1215 | GCGGCAGGAGGCACAAGAA | 3266 | UUCUUGUGCCUCCUGCCGC |
| siRNA 1224 | 1224 | GGCACAAGAAGAGGACGUU | 3275 | AACGUCCUCUUCUUGUGCC |
| siRNA 1225 | 1225 | GCACAAGAAGAGGACGUUC | 3276 | GAACGUCCUCUUCUUGUGC |
| siRNA 1226 | 1226 | CACAAGAAGAGGACGUUCC | 3277 | GGAACGUCCUCUUCUUGUG |
| siRNA 1235 | 1235 | AGGACGUUCCUGCGGCCAC | 3286 | GUGGCCGCAGGAACGUCCU |
| siRNA 1236 | 1236 | GGACGUUCCUGCGGCCACG | 3287 | CGUGGCCGCAGGAACGUCC |
| siRNA 1238 | 1238 | ACGUUCCUGCGGCCACGUA | 3289 | UACGUGGCCGCAGGAACGU |
| siRNA 1240 | 1240 | GUUCCUGCGGCCACGUAUC | 3291 | GAUACGUGGCCGCAGGAAC |
| siRNA 1242 | 1242 | UCCUGCGGCCACGUAUCAU | 3293 | AUGAUACGUGGCCGCAGGA |
| siRNA 1243 | 1243 | CCUGCGGCCACGUAUCAUC | 3294 | GAUGAUACGUGGCCGCAGG |
| SiRNA 1244 | 1244 | CUGCGGCCACGUAUCAUCG | 3295 | CGAUGAUACGUGGCCGCAG |
| siRNA 1246 | 1246 | GCGGCCACGUAUCAUCGGC | 3297 | GCCGAUGAUACGUGGCCGC |
| siRNA 1247 | 1247 | CGGCCACGUAUCAUCGGCG | 3298 | CGCCGAUGAUACGUGGCCG |
| siRNA 1248 | 1248 | GGCCACGUAUCAUCGGCGG | 3299 | CCGCCGAUGAUACGUGGCC |
| siRNA 1249 | 1249 | GCCACGUAUCAUCGGCGGC | 3300 | GCCGCCGAUGAUACGUGGC |
| siRNA 1250 | 1250 | CCACGUAUCAUCGGCGGCU | 3301 | AGCCGCCGAUGAUACGUGG |
| siRNA 1251 | 1251 | CACGUAUCAUCGGCGGCUC | 3302 | GAGCCGCCGAUGAUACGUG |
| siRNA 1274 | 1274 | UCGCUGCCCGGCUCGCACC | 3325 | GGUGCGAGCCGGGCAGCGA |
| SiRNA 1299 | 1299 | UGGCCGCCAUCUACAUCGG | 3350 | CCGAUGUAGAUGGCGGCCA |
| siRNA 1303 | 1303 | CGCCAUCUACAUCGGGGAC | 3354 | GUCCCCGAUGUAGAUGGCG |
| siRNA 1307 | 1307 | AUCUACAUCGGGGACAGCU | 3358 | AGCUGUCCCCGAUGUAGAU |
| siRNA 1309 | 1309 | CUACAUCGGGGACAGCUUC | 3360 | GAAGCUGUCCCCGAUGUAG |
| siRNA 1310 | 1310 | UACAUCGGGGACAGCUUCU | 3361 | AGAAGCUGUCCCCGAUGUA |
| siRNA 1312 | 1312 | CAUCGGGGACAGCUUCUGC | 3363 | GCAGAAGCUGUCCCCGAUG |
| siRNA 1314 | 1314 | UCGGGGACAGCUUCUGCGC | 3365 | GCGCAGAAGCUGUCCCCGA |
| siRNA 1315 | 1315 | CGGGGACAGCUUCUGCGCC | 3366 | GGCGCAGAAGCUGUCCCCG |
| siRNA 1318 | 1318 | GGACAGCUUCUGCGCCGGG | 3369 | CCCGGCGCAGAAGCUGUCC |
| siRNA 1319 | 1319 | GACAGCUUCUGCGCCGGGA | 3370 | UCCCGGCGCAGAAGCUGUC |
| siRNA 1352 | 1352 | UGCUGGGUGGUGUCGGCCG | 3403 | CGGCCGACACCACCCAGCA |
| siRNA 1353 | 1353 | GCUGGGUGGUGUCGGCCGC | 3404 | GCGGCCGACACCACCCAGC |
| siRNA 1379 | 1379 | UUCUCCCACAGCCCCCCCA | 3430 | UGGGGGGGCUGUGGGAGAA |
| siRNA 1389 | 1389 | GCCCCCCCAGGGACAGCGU | 3440 | ACGCUGUCCCUGGGGGGGC |
| siRNA 1390 | 1390 | CCCCCCCAGGGACAGCGUC | 3441 | GACGCUGUCCCUGGGGGGG |
| siRNA 1392 | 1392 | CCCCCAGGGACAGCGUCUC | 3443 | GAGACGCUGUCCCUGGGGG |
| siRNA 1424 | 1424 | CAGCACUUCUUCAACCGCA | 3475 | UGCGGUUGAAGAAGUGCUG |
| siRNA 1427 | 1427 | CACUUCUUCAACCGCACGA | 3478 | UCGUGCGGUUGAAGAAGUG |
| siRNA 1428 | 1428 | ACUUCUUCAACCGCACGAC | 3479 | GUCGUGCGGUUGAAGAAGU |
| SiRNA 1429 | 1429 | CUUCUUCAACCGCACGACG | 3480 | CGUCGUGCGGUUGAAGAAG |
| siRNA 1430 | 1430 | UUCUUCAACCGCACGACGG | 3481 | CCGUCGUGCGGUUGAAGAA |
| siRNA 1431 | 1431 | UCUUCAACCGCACGACGGA | 3482 | UCCGUCGUGCGGUUGAAGA |
| siRNA 1432 | 1432 | CUUCAACCGCACGACGGAC | 3483 | GUCCGUCGUGCGGUUGAAG |
| siRNA 1433 | 1433 | UUCAACCGCACGACGGACG | 3484 | CGUCCGUCGUGCGGUUGAA |
| siRNA 1434 | 1434 | UCAACCGCACGACGGACGU | 3485 | ACGUCCGUCGUGCGGUUGA |
| siRNA 1435 | 1435 | CAACCGCACGACGGACGUG | 3486 | CACGUCCGUCGUGCGGUUG |
| SiRNA 1436 | 1436 | AACCGCACGACGGACGUGA | 3487 | UCACGUCCGUCGUGCGGUU |
| siRNA 1438 | 1438 | CCGCACGACGGACGUGACG | 3489 | CGUCACGUCCGUCGUGCGG |
| siRNA 1439 | 1439 | CGCACGACGGACGUGACGC | 3490 | GCGUCACGUCCGUCGUGCG |
| siRNA 1440 | 1440 | GCACGACGGACGUGACGCA | 3491 | UGCGUCACGUCCGUCGUGC |
| siRNA 1441 | 1441 | CACGACGGACGUGACGCAG | 3492 | CUGCGUCACGUCCGUCGUG |
| siRNA 1442 | 1442 | ACGACGGACGUGACGCAGA | 3493 | UCUGCGUCACGUCCGUCGU |
| siRNA 1443 | 1443 | CGACGGACGUGACGCAGAC | 3494 | GUCUGCGUCACGUCCGUCG |
| siRNA 1445 | 1445 | ACGGACGUGACGCAGACCU | 3496 | AGGUCUGCGUCACGUCCGU |
| siRNA 1446 | 1446 | CGGACGUGACGCAGACCUU | 3497 | AAGGUCUGCGUCACGUCCG |
| siRNA 1447 | 1447 | GGACGUGACGCAGACCUUC | 3498 | GAAGGUCUGCGUCACGUCC |
| SiRNA 1449 | 1449 | ACGUGACGCAGACCUUCGG | 3500 | CCGAAGGUCUGCGUCACGU |
| siRNA 1452 | 1452 | UGACGCAGACCUUCGGCAU | 3503 | AUGCCGAAGGUCUGCGUCA |
| siRNA 1453 | 1453 | GACGCAGACCUUCGGCAUC | 3504 | GAUGCCGAAGGUCUGCGUC |
| siRNA 1455 | 1455 | CGCAGACCUUCGGCAUCGA | 3506 | UCGAUGCCGAAGGUCUGCG |
| siRNA 1456 | 1456 | GCAGACCUUCGGCAUCGAG | 3507 | CUCGAUGCCGAAGGUCUGC |
| siRNA 1459 | 1459 | GACCUUCGGCAUCGAGAAG | 3510 | CUUCUCGAUGCCGAAGGUC |
| siRNA 1460 | 1460 | ACCUUCGGCAUCGAGAAGU | 3511 | ACUUCUCGAUGCCGAAGGU |
| siRNA 1463 | 1463 | UUCGGCAUCGAGAAGUACA | 3514 | UGUACUUCUCGAUGCCGAA |
| SiRNA 1464 | 1464 | UCGGCAUCGAGAAGUACAU | 3515 | AUGUACUUCUCGAUGCCGA |
| siRNA 1469 | 1469 | AUCGAGAAGUACAUCCCGU | 3520 | ACGGGAUGUACUUCUCGAU |
| SiRNA 1470 | 1470 | UCGAGAAGUACAUCCCGUA | 3521 | UACGGGAUGUACUUCUCGA |
| siRNA 1471 | 1471 | CGAGAAGUACAUCCCGUAC | 3522 | GUACGGGAUGUACUUCUCG |
| SiRNA 1474 | 1474 | GAAGUACAUCCCGUACACC | 3525 | GGUGUACGGGAUGUACUUC |
| siRNA 1475 | 1475 | AAGUACAUCCCGUACACCC | 3526 | GGGUGUACGGGAUGUACUU |
| siRNA 1476 | 1476 | AGUACAUCCCGUACACCCU | 3527 | AGGGUGUACGGGAUGUACU |
| siRNA 1477 | 1477 | GUACAUCCCGUACACCCUG | 3528 | CAGGGUGUACGGGAUGUAC |
| siRNA 1478 | 1478 | UACAUCCCGUACACCCUGU | 3529 | ACAGGGUGUACGGGAUGUA |
| siRNA 1504 | 1504 | GUUCAACCCCAGCGACCAC | 3555 | GUGGUCGCUGGGGUUGAAC |
| siRNA 1506 | 1506 | UCAACCCCAGCGACCACGA | 3557 | UCGUGGUCGCUGGGGUUGA |
| siRNA 1507 | 1507 | CAACCCCAGCGACCACGAC | 3558 | GUCGUGGUCGCUGGGGUUG |
| siRNA 1509 | 1509 | ACCCCAGCGACCACGACCU | 3560 | AGGUCGUGGUCGCUGGGGU |
| siRNA 1512 | 1512 | CCAGCGACCACGACCUCGU | 3563 | ACGAGGUCGUGGUCGCUGG |
| siRNA 1513 | 1513 | CAGCGACCACGACCUCGUC | 3564 | GACGAGGUCGUGGUCGCUG |
| siRNA 1516 | 1516 | CGACCACGACCUCGUCCUG | 3567 | CAGGACGAGGUCGUGGUCG |
| siRNA 1518 | 1518 | ACCACGACCUCGUCCUGAU | 3569 | AUCAGGACGAGGUCGUGGU |
| siRNA 1519 | 1519 | CCACGACCUCGUCCUGAUC | 3570 | GAUCAGGACGAGGUCGUGG |
| siRNA 1521 | 1521 | ACGACCUCGUCCUGAUCCG | 3572 | CGGAUCAGGACGAGGUCGU |
| siRNA 1523 | 1523 | GACCUCGUCCUGAUCCGGC | 3574 | GCCGGAUCAGGACGAGGUC |
| siRNA 1524 | 1524 | ACCUCGUCCUGAUCCGGCU | 3575 | AGCCGGAUCAGGACGAGGU |
| siRNA 1528 | 1528 | CGUCCUGAUCCGGCUGAAG | 3579 | CUUCAGCCGGAUCAGGACG |
| siRNA 1562 | 1562 | UGUGCCACACGCUCGCAGU | 3613 | ACUGCGAGCGUGUGGCACA |
| siRNA 1563 | 1563 | GUGCCACACGCUCGCAGUU | 3614 | AACUGCGAGCGUGUGGCAC |
| siRNA 1564 | 1564 | UGCCACACGCUCGCAGUUC | 3615 | GAACUGCGAGCGUGUGGCA |
| siRNA 1565 | 1565 | GCCACACGCUCGCAGUUCG | 3616 | CGAACUGCGAGCGUGUGGC |
| siRNA 1566 | 1566 | CCACACGCUCGCAGUUCGU | 3617 | ACGAACUGCGAGCGUGUGG |
| siRNA 1567 | 1567 | CACACGCUCGCAGUUCGUG | 3618 | CACGAACUGCGAGCGUGUG |
| siRNA 1568 | 1568 | ACACGCUCGCAGUUCGUGC | 3619 | GCACGAACUGCGAGCGUGU |
| siRNA 1572 | 1572 | GCUCGCAGUUCGUGCAGCC | 3623 | GGCUGCACGAACUGCGAGC |
| siRNA 1574 | 1574 | UCGCAGUUCGUGCAGCCCA | 3625 | UGGGCUGCACGAACUGCGA |
| siRNA 1615 | 1615 | CACCUUCCCCGCAGGACAC | 3666 | GUGUCCUGCGGGGAAGGUG |
| siRNA 1616 | 1616 | ACCUUCCCCGCAGGACACA | 3667 | UGUGUCCUGCGGGGAAGGU |
| siRNA 1617 | 1617 | CCUUCCCCGCAGGACACAA | 3668 | UUGUGUCCUGCGGGGAAGG |
| siRNA 1619 | 1619 | UUCCCCGCAGGACACAAGU | 3670 | ACUUGUGUCCUGCGGGGAA |
| siRNA 1621 | 1621 | CCCCGCAGGACACAAGUGC | 3672 | GCACUUGUGUCCUGCGGGG |
| siRNA 1622 | 1622 | CCCGCAGGACACAAGUGCC | 3673 | GGCACUUGUGUCCUGCGGG |
| siRNA 1623 | 1623 | CCGCAGGACACAAGUGCCA | 3674 | UGGCACUUGUGUCCUGCGG |
| siRNA 1624 | 1624 | CGCAGGACACAAGUGCCAG | 3675 | CUGGCACUUGUGUCCUGCG |
| siRNA 1628 | 1628 | GGACACAAGUGCCAGAUUG | 3679 | CAAUCUGGCACUUGUGUCC |
| siRNA 1630 | 1630 | ACACAAGUGCCAGAUUGCG | 3681 | CGCAAUCUGGCACUUGUGU |
| siRNA 1631 | 1631 | CACAAGUGCCAGAUUGCGG | 3682 | CCGCAAUCUGGCACUUGUG |
| siRNA 1635 | 1635 | AGUGCCAGAUUGCGGGCUG | 3686 | CAGCCCGCAAUCUGGCACU |
| siRNA 1636 | 1636 | GUGCCAGAUUGCGGGCUGG | 3687 | CCAGCCCGCAAUCUGGCAC |
| siRNA 1637 | 1637 | UGCCAGAUUGCGGGCUGGG | 3688 | CCCAGCCCGCAAUCUGGCA |
| siRNA 1643 | 1643 | AUUGCGGGCUGGGGCCACU | 3694 | AGUGGCCCCAGCCCGCAAU |
| siRNA 1651 | 1651 | CUGGGGCCACUUGGAUGAG | 3702 | CUCAUCCAAGUGGCCCCAG |
| siRNA 1662 | 1662 | UGGAUGAGAACGUGAGCGG | 3713 | CCGCUCACGUUCUCAUCCA |
| siRNA 1663 | 1663 | GGAUGAGAACGUGAGCGGC | 3714 | GCCGCUCACGUUCUCAUCC |
| siRNA 1664 | 1664 | GAUGAGAACGUGAGCGGCU | 3715 | AGCCGCUCACGUUCUCAUC |
| siRNA 1672 | 1672 | CGUGAGCGGCUACUCCAGC | 3723 | GCUGGAGUAGCCGCUCACG |
| siRNA 1707 | 1707 | UGGUCCCCCUGGUCGCCGA | 3758 | UCGGCGACCAGGGGGACCA |
| SiRNA 1708 | 1708 | GGUCCCCCUGGUCGCCGAC | 3759 | GUCGGCGACCAGGGGGACC |
| SiRNA 1709 | 1709 | GUCCCCCUGGUCGCCGACC | 3760 | GGUCGGCGACCAGGGGGAC |
| siRNA 1712 | 1712 | CCCCUGGUCGCCGACCACA | 3763 | UGUGGUCGGCGACCAGGGG |
| siRNA 1713 | 1713 | CCCUGGUCGCCGACCACAA | 3764 | UUGUGGUCGGCGACCAGGG |
| siRNA 1715 | 1715 | CUGGUCGCCGACCACAAGU | 3766 | ACUUGUGGUCGGCGACCAG |
| siRNA 1716 | 1716 | UGGUCGCCGACCACAAGUG | 3767 | CACUUGUGGUCGGCGACCA |
| siRNA 1717 | 1717 | GGUCGCCGACCACAAGUGC | 3768 | GCACUUGUGGUCGGCGACC |
| siRNA 1718 | 1718 | GUCGCCGACCACAAGUGCA | 3769 | UGCACUUGUGGUCGGCGAC |
| siRNA 1722 | 1722 | CCGACCACAAGUGCAGCAG | 3773 | CUGCUGCACUUGUGGUCGG |
| siRNA 1737 | 1737 | GCAGCCCUGAGGUCUACGG | 3788 | CCGUAGACCUCAGGGCUGC |
| siRNA 1739 | 1739 | AGCCCUGAGGUCUACGGCG | 3790 | CGCCGUAGACCUCAGGGCU |
| siRNA 1741 | 1741 | CCCUGAGGUCUACGGCGCC | 3792 | GGCGCCGUAGACCUCAGGG |
| siRNA 1743 | 1743 | CUGAGGUCUACGGCGCCGA | 3794 | UCGGCGCCGUAGACCUCAG |
| siRNA 1745 | 1745 | GAGGUCUACGGCGCCGACA | 3796 | UGUCGGCGCCGUAGACCUC |
| SiRNA 1749 | 1749 | UCUACGGCGCCGACAUCAG | 3800 | CUGAUGUCGGCGCCGUAGA |
| siRNA 1751 | 1751 | UACGGCGCCGACAUCAGCC | 3802 | GGCUGAUGUCGGCGCCGUA |
| siRNA 1752 | 1752 | ACGGCGCCGACAUCAGCCC | 3803 | GGGCUGAUGUCGGCGCCGU |
| siRNA 1753 | 1753 | CGGCGCCGACAUCAGCCCC | 3804 | GGGGCUGAUGUCGGCGCCG |
| siRNA 1759 | 1759 | CGACAUCAGCCCCAACAUG | 3810 | CAUGUUGGGGCUGAUGUCG |
| siRNA 1766 | 1766 | AGCCCCAACAUGCUCUGUG | 3817 | CACAGAGCAUGUUGGGGCU |
| siRNA 1770 | 1770 | CCAACAUGCUCUGUGCCGG | 3821 | CCGGCACAGAGCAUGUUGG |
| siRNA 1780 | 1780 | CUGUGCCGGCUACUUCGAC | 3831 | GUCGAAGUAGCCGGCACAG |
| siRNA 1781 | 1781 | UGUGCCGGCUACUUCGACU | 3832 | AGUCGAAGUAGCCGGCACA |
| siRNA 1782 | 1782 | GUGCCGGCUACUUCGACUG | 3833 | CAGUCGAAGUAGCCGGCAC |
| siRNA 1783 | 1783 | UGCCGGCUACUUCGACUGC | 3834 | GCAGUCGAAGUAGCCGGCA |
| siRNA 1785 | 1785 | CCGGCUACUUCGACUGCAA | 3836 | UUGCAGUCGAAGUAGCCGG |
| siRNA 1786 | 1786 | CGGCUACUUCGACUGCAAG | 3837 | CUUGCAGUCGAAGUAGCCG |
| siRNA 1788 | 1788 | GCUACUUCGACUGCAAGUC | 3839 | GACUUGCAGUCGAAGUAGC |
| siRNA 1789 | 1789 | CUACUUCGACUGCAAGUCC | 3840 | GGACUUGCAGUCGAAGUAG |
| siRNA 1791 | 1791 | ACUUCGACUGCAAGUCCGA | 3842 | UCGGACUUGCAGUCGAAGU |
| siRNA 1792 | 1792 | CUUCGACUGCAAGUCCGAC | 3843 | GUCGGACUUGCAGUCGAAG |
| siRNA 1793 | 1793 | UUCGACUGCAAGUCCGACG | 3844 | CGUCGGACUUGCAGUCGAA |
| siRNA 1794 | 1794 | UCGACUGCAAGUCCGACGC | 3845 | GCGUCGGACUUGCAGUCGA |
| siRNA 1795 | 1795 | CGACUGCAAGUCCGACGCC | 3846 | GGCGUCGGACUUGCAGUCG |
| siRNA 1797 | 1797 | ACUGCAAGUCCGACGCCUG | 3848 | CAGGCGUCGGACUUGCAGU |
| siRNA 1798 | 1798 | CUGCAAGUCCGACGCCUGC | 3849 | GCAGGCGUCGGACUUGCAG |
| siRNA 1799 | 1799 | UGCAAGUCCGACGCCUGCC | 3850 | GGCAGGCGUCGGACUUGCA |
| siRNA 1840 | 1840 | GGCCUGCGAGAAGAACGGC | 3891 | GCCGUUCUUCUCGCAGGCC |
| siRNA 1843 | 1843 | CUGCGAGAAGAACGGCGUG | 3894 | CACGCCGUUCUUCUCGCAG |
| siRNA 1849 | 1849 | GAAGAACGGCGUGGCUUAC | 3900 | GUAAGCCACGCCGUUCUUC |
| siRNA 1850 | 1850 | AAGAACGGCGUGGCUUACC | 3901 | GGUAAGCCACGCCGUUCUU |
| siRNA 1851 | 1851 | AGAACGGCGUGGCUUACCU | 3902 | AGGUAAGCCACGCCGUUCU |
| siRNA 1856 | 1856 | GGCGUGGCUUACCUCUACG | 3907 | CGUAGAGGUAAGCCACGCC |
| siRNA 1857 | 1857 | GCGUGGCUUACCUCUACGG | 3908 | CCGUAGAGGUAAGCCACGC |
| siRNA 1858 | 1858 | CGUGGCUUACCUCUACGGC | 3909 | GCCGUAGAGGUAAGCCACG |
| siRNA 1859 | 1859 | GUGGCUUACCUCUACGGCA | 3910 | UGCCGUAGAGGUAAGCCAC |
| siRNA 1860 | 1860 | UGGCUUACCUCUACGGCAU | 3911 | AUGCCGUAGAGGUAAGCCA |
| siRNA 1861 | 1861 | GGCUUACCUCUACGGCAUC | 3912 | GAUGCCGUAGAGGUAAGCC |
| SiRNA 1862 | 1862 | GCUUACCUCUACGGCAUCA | 3913 | UGAUGCCGUAGAGGUAAGC |
| siRNA 1863 | 1863 | CUUACCUCUACGGCAUCAU | 3914 | AUGAUGCCGUAGAGGUAAG |
| siRNA 1864 | 1864 | UUACCUCUACGGCAUCAUC | 3915 | GAUGAUGCCGUAGAGGUAA |
| siRNA 1895 | 1895 | GGCUGCGGGCGGCUCCACA | 3946 | UGUGGAGCCGCCCGCAGCC |
| siRNA 1896 | 1896 | GCUGCGGGCGGCUCCACAA | 3947 | UUGUGGAGCCGCCCGCAGC |
| siRNA 1897 | 1897 | CUGCGGGCGGCUCCACAAG | 3948 | CUUGUGGAGCCGCCCGCAG |
| siRNA 1900 | 1900 | CGGGCGGCUCCACAAGCCG | 3951 | CGGCUUGUGGAGCCGCCCG |
| siRNA 1901 | 1901 | GGGCGGCUCCACAAGCCGG | 3952 | CCGGCUUGUGGAGCCGCCC |
| siRNA 1902 | 1902 | GGCGGCUCCACAAGCCGGG | 3953 | CCCGGCUUGUGGAGCCGCC |
| siRNA 1903 | 1903 | GCGGCUCCACAAGCCGGGG | 3954 | CCCCGGCUUGUGGAGCCGC |
| siRNA 1904 | 1904 | CGGCUCCACAAGCCGGGGG | 3955 | CCCCCGGCUUGUGGAGCCG |
| siRNA 1906 | 1906 | GCUCCACAAGCCGGGGGUC | 3957 | GACCCCCGGCUUGUGGAGC |
| siRNA 1908 | 1908 | UCCACAAGCCGGGGGUCUA | 3959 | UAGACCCCCGGCUUGUGGA |
| siRNA 1909 | 1909 | CCACAAGCCGGGGGUCUAC | 3960 | GUAGACCCCCGGCUUGUGG |
| siRNA 1910 | 1910 | CACAAGCCGGGGGUCUACA | 3961 | UGUAGACCCCCGGCUUGUG |
| siRNA 1917 | 1917 | CGGGGGUCUACACCCGCGU | 3968 | ACGCGGGUGUAGACCCCCG |
| siRNA 1927 | 1927 | CACCCGCGUGGCCAACUAU | 3978 | AUAGUUGGCCACGCGGGUG |
| SiRNA 1928 | 1928 | ACCCGCGUGGCCAACUAUG | 3979 | CAUAGUUGGCCACGCGGGU |
| SiRNA 1929 | 1929 | CCCGCGUGGCCAACUAUGU | 3980 | ACAUAGUUGGCCACGCGGG |
| SiRNA 1937 | 1937 | GCCAACUAUGUGGACUGGA | 3988 | UCCAGUCCACAUAGUUGGC |
| siRNA 1966 | 1966 | GAUACGGCCUCCCAGGCGG | 4017 | CCGCCUGGGAGGCCGUAUC |
| siRNA 1967 | 1967 | AUACGGCCUCCCAGGCGGC | 4018 | GCCGCCUGGGAGGCCGUAU |
| siRNA 1974 | 1974 | CUCCCAGGCGGCUUGUGGC | 4025 | GCCACAAGCCGCCUGGGAG |
| siRNA 1979 | 1979 | AGGCGGCUUGUGGCUCCCU | 4030 | AGGGAGCCACAAGCCGCCU |
| siRNA 1984 | 1984 | GCUUGUGGCUCCCUCCUGA | 4035 | UCAGGAGGGAGCCACAAGC |
| siRNA 1999 | 1999 | CUGACCCUCCAGCGGGACA | 4050 | UGUCCCGCUGGAGGGUCAG |
| siRNA 2001 | 2001 | GACCCUCCAGCGGGACACC | 4052 | GGUGUCCCGCUGGAGGGUC |
| siRNA 2010 | 2010 | GCGGGACACCCUGGUUCCC | 4061 | GGGAACCAGGGUGUCCCGC |
| siRNA 1999 | 1999 | CUGACCCUCCAGCGGGACA | 4050 | UGUCCCGCUGGAGGGUCAG |
| siRNA 2001 | 2001 | GACCCUCCAGCGGGACACC | 4052 | GGUGUCCCGCUGGAGGGUC |
| siRNA 2010 | 2010 | GCGGGACACCCUGGUUCCC | 4061 | GGGAACCAGGGUGUCCCGC |

The siRNAs in subset A had the following characteristics: Cross-reactivity: With 19mer in human HGFAC mRNA; Specificity category: For human: AS2 or better, SS3 or better; and miRNA seeds: AS+SS strand: seed region not conserved in human, mouse, and rat and not present in >4 species; Off-target frequency: ≤30 human off-targets matched with 2 mismatches in antisense strand; and SNPs: siRNA target sites do not harbor SNPs with a MAF≥1% (pos. 2-18).

The siRNA sequences in subset A were selected for more stringent specificity to yield subset B. Subset B includes 377 siRNAs whose base sequences are shown in Table B.

TABLE B
Subset B

| | SEQ ID | sense strand | SEQ ID | antisense strand |
|---|---|---|---|---|
| siRNA NO: | NO: | sequence (5′-3′) | NO: | sequence (5′-3′) |
| siRNA 26 | 26 | UCAGGAGCCAUGGGGCGCU | 2077 | AGCGCCCCAUGGCUCCUGA |
| siRNA 114 | 114 | UGCUGCUGCCACGGGGGUU | 2165 | AACCCCCGUGGCAGCAGCA |
| siRNA 116 | 116 | CUGCUGCCACGGGGGUUCC | 2167 | GGAACCCCCGUGGCAGCAG |
| siRNA 118 | 118 | GCUGCCACGGGGGUUCCAG | 2169 | CUGGAACCCCCGUGGCAGC |
| siRNA 122 | 122 | CCACGGGGGUUCCAGCCCC | 2173 | GGGGCUGGAACCCCCGUGG |
| siRNA 136 | 136 | GCCCCAGCCUGGCGGGAAC | 2187 | GUUCCCGCCAGGCUGGGGC |
| siRNA 137 | 137 | CCCCAGCCUGGCGGGAACC | 2188 | GGUUCCCGCCAGGCUGGGG |
| siRNA 140 | 140 | CAGCCUGGCGGGAACCGUA | 2191 | UACGGUUCCCGCCAGGCUG |
| siRNA 141 | 141 | AGCCUGGCGGGAACCGUAC | 2192 | GUACGGUUCCCGCCAGGCU |
| siRNA 143 | 143 | CCUGGCGGGAACCGUACGG | 2194 | CCGUACGGUUCCCGCCAGG |
| siRNA 144 | 144 | CUGGCGGGAACCGUACGGA | 2195 | UCCGUACGGUUCCCGCCAG |
| siRNA 145 | 145 | UGGCGGGAACCGUACGGAG | 2196 | CUCCGUACGGUUCCCGCCA |
| siRNA 146 | 146 | GGCGGGAACCGUACGGAGU | 2197 | ACUCCGUACGGUUCCCGCC |
| siRNA 149 | 149 | GGGAACCGUACGGAGUCCC | 2200 | GGGACUCCGUACGGUUCCC |
| siRNA 150 | 150 | GGAACCGUACGGAGUCCCC | 2201 | GGGGACUCCGUACGGUUCC |
| siRNA 152 | 152 | AACCGUACGGAGUCCCCAG | 2203 | CUGGGGACUCCGUACGGUU |
| siRNA 154 | 154 | CCGUACGGAGUCCCCAGAA | 2205 | UUCUGGGGACUCCGUACGG |
| siRNA 155 | 155 | CGUACGGAGUCCCCAGAAC | 2206 | GUUCUGGGGACUCCGUACG |
| siRNA 158 | 158 | ACGGAGUCCCCAGAACCUA | 2209 | UAGGUUCUGGGGACUCCGU |
| siRNA 160 | 160 | GGAGUCCCCAGAACCUAAU | 2211 | AUUAGGUUCUGGGGACUCC |
| siRNA 165 | 165 | CCCCAGAACCUAAUGCCAC | 2216 | GUGGCAUUAGGUUCUGGGG |
| siRNA 166 | 166 | CCCAGAACCUAAUGCCACA | 2217 | UGUGGCAUUAGGUUCUGGG |
| siRNA 167 | 167 | CCAGAACCUAAUGCCACAG | 2218 | CUGUGGCAUUAGGUUCUGG |
| siRNA 172 | 172 | ACCUAAUGCCACAGCGACC | 2223 | GGUCGCUGUGGCAUUAGGU |
| siRNA 173 | 173 | CCUAAUGCCACAGCGACCC | 2224 | GGGUCGCUGUGGCAUUAGG |
| siRNA 199 | 199 | CCCCACUAUCCUGGUGACC | 2250 | GGUCACCAGGAUAGUGGGG |
| siRNA 202 | 202 | CACUAUCCUGGUGACCUCU | 2253 | AGAGGUCACCAGGAUAGUG |
| siRNA 203 | 203 | ACUAUCCUGGUGACCUCUG | 2254 | CAGAGGUCACCAGGAUAGU |
| siRNA 205 | 205 | UAUCCUGGUGACCUCUGUG | 2256 | CACAGAGGUCACCAGGAUA |
| siRNA 232 | 232 | GACCCCAGCAACAAGUGCU | 2283 | AGCACUUGUUGCUGGGGUC |
| siRNA 294 | 294 | CCAGGGCAGUUCCCUCGAG | 2345 | CUCGAGGGAACUGCCCUGG |
| siRNA 295 | 295 | CAGGGCAGUUCCCUCGAGC | 2346 | GCUCGAGGGAACUGCCCUG |
| siRNA 296 | 296 | AGGGCAGUUCCCUCGAGCA | 2347 | UGCUCGAGGGAACUGCCCU |
| siRNA 301 | 301 | AGUUCCCUCGAGCAGUAGC | 2352 | GCUACUGCUCGAGGGAACU |
| siRNA 303 | 303 | UUCCCUCGAGCAGUAGCCC | 2354 | GGGCUACUGCUCGAGGGAA |
| siRNA 304 | 304 | UCCCUCGAGCAGUAGCCCC | 2355 | GGGGCUACUGCUCGAGGGA |
| siRNA 305 | 305 | CCCUCGAGCAGUAGCCCCC | 2356 | GGGGGCUACUGCUCGAGGG |
| siRNA 315 | 315 | GUAGCCCCCAGGCCCAAGC | 2366 | GCUUGGGCCUGGGGGCUAC |
| siRNA 328 | 328 | CCAAGCACUCACCGAGGAC | 2379 | GUCCUCGGUGAGUGCUUGG |
| siRNA 334 | 334 | ACUCACCGAGGACGGGAGG | 2385 | CCUCCCGUCCUCGGUGAGU |
| siRNA 335 | 335 | CUCACCGAGGACGGGAGGC | 2386 | GCCUCCCGUCCUCGGUGAG |
| siRNA 336 | 336 | UCACCGAGGACGGGAGGCC | 2387 | GGCCUCCCGUCCUCGGUGA |
| siRNA 360 | 360 | GGUUCCCCUUCCGCUACGG | 2411 | CCGUAGCGGAAGGGGAACC |
| siRNA 361 | 361 | GUUCCCCUUCCGCUACGGG | 2412 | CCCGUAGCGGAAGGGGAAC |
| siRNA 362 | 362 | UUCCCCUUCCGCUACGGGG | 2413 | CCCCGUAGCGGAAGGGGAA |
| siRNA 363 | 363 | UCCCCUUCCGCUACGGGGG | 2414 | CCCCCGUAGCGGAAGGGGA |
| siRNA 364 | 364 | CCCCUUCCGCUACGGGGGC | 2415 | GCCCCCGUAGCGGAAGGGG |
| siRNA 367 | 367 | CUUCCGCUACGGGGGCCGC | 2418 | GCGGCCCCCGUAGCGGAAG |
| siRNA 371 | 371 | CGCUACGGGGGCCGCAUGC | 2422 | GCAUGCGGCCCCCGUAGCG |
| siRNA 372 | 372 | GCUACGGGGGCCGCAUGCU | 2423 | AGCAUGCGGCCCCCGUAGC |
| siRNA 373 | 373 | CUACGGGGGCCGCAUGCUG | 2424 | CAGCAUGCGGCCCCCGUAG |
| siRNA 374 | 374 | UACGGGGGCCGCAUGCUGC | 2425 | GCAGCAUGCGGCCCCCGUA |
| siRNA 378 | 378 | GGGGCCGCAUGCUGCAUGC | 2429 | GCAUGCAGCAUGCGGCCCC |
| siRNA 389 | 389 | CUGCAUGCCUGCACUUCGG | 2440 | CCGAAGUGCAGGCAUGCAG |
| siRNA 390 | 390 | UGCAUGCCUGCACUUCGGA | 2441 | UCCGAAGUGCAGGCAUGCA |
| siRNA 391 | 391 | GCAUGCCUGCACUUCGGAG | 2442 | CUCCGAAGUGCAGGCAUGC |
| siRNA 395 | 395 | GCCUGCACUUCGGAGGGCA | 2446 | UGCCCUCCGAAGUGCAGGC |
| siRNA 398 | 398 | UGCACUUCGGAGGGCAGUG | 2449 | CACUGCCCUCCGAAGUGCA |
| siRNA 431 | 431 | UGUGCCACAACUCACAACU | 2482 | AGUUGUGAGUUGUGGCACA |
| siRNA 434 | 434 | GCCACAACUCACAACUACG | 2485 | CGUAGUUGUGAGUUGUGGC |
| siRNA 436 | 436 | CACAACUCACAACUACGAC | 2487 | GUCGUAGUUGUGAGUUGUG |
| siRNA 437 | 437 | ACAACUCACAACUACGACC | 2488 | GGUCGUAGUUGUGAGUUGU |
| siRNA 438 | 438 | CAACUCACAACUACGACCG | 2489 | CGGUCGUAGUUGUGAGUUG |
| siRNA 442 | 442 | UCACAACUACGACCGGGAC | 2493 | GUCCCGGUCGUAGUUGUGA |
| siRNA 443 | 443 | CACAACUACGACCGGGACA | 2494 | UGUCCCGGUCGUAGUUGUG |
| siRNA 445 | 445 | CAACUACGACCGGGACAGG | 2496 | CCUGUCCCGGUCGUAGUUG |
| siRNA 446 | 446 | AACUACGACCGGGACAGGG | 2497 | CCCUGUCCCGGUCGUAGUU |
| siRNA 469 | 469 | GGGCUACUGUGUGGAGGCC | 2520 | GGCCUCCACACAGUAGCCC |
| siRNA 472 | 472 | CUACUGUGUGGAGGCCACC | 2523 | GGUGGCCUCCACACAGUAG |
| siRNA 473 | 473 | UACUGUGUGGAGGCCACCC | 2524 | GGGUGGCCUCCACACAGUA |
| siRNA 530 | 530 | UCCGGCCCCUGCCUCAAUG | 2581 | CAUUGAGGCAGGGGCCGGA |
| siRNA 531 | 531 | CCGGCCCCUGCCUCAAUGG | 2582 | CCAUUGAGGCAGGGGCCGG |
| siRNA 549 | 549 | GAGGCUCCUGCUCCAAUAC | 2600 | GUAUUGGAGCAGGAGCCUC |
| siRNA 558 | 558 | GCUCCAAUACCCAGGACCC | 2609 | GGGUCCUGGGUAUUGGAGC |
| siRNA 562 | 562 | CAAUACCCAGGACCCCCAG | 2613 | CUGGGGGUCCUGGGUAUUG |
| siRNA 564 | 564 | AUACCCAGGACCCCCAGUC | 2615 | GACUGGGGGUCCUGGGUAU |
| siRNA 569 | 569 | CAGGACCCCCAGUCCUAUC | 2620 | GAUAGGACUGGGGGUCCUG |
| siRNA 575 | 575 | CCCCAGUCCUAUCACUGCA | 2626 | UGCAGUGAUAGGACUGGGG |
| siRNA 601 | 601 | CCGGGCCUUCACCGGCAAG | 2652 | CUUGCCGGUGAAGGCCCGG |
| siRNA 602 | 602 | CGGGCCUUCACCGGCAAGG | 2653 | CCUUGCCGGUGAAGGCCCG |
| siRNA 603 | 603 | GGGCCUUCACCGGCAAGGA | 2654 | UCCUUGCCGGUGAAGGCCC |
| siRNA 604 | 604 | GGCCUUCACCGGCAAGGAC | 2655 | GUCCUUGCCGGUGAAGGCC |
| siRNA 610 | 610 | CACCGGCAAGGACUGCGGC | 2661 | GCCGCAGUCCUUGCCGGUG |
| siRNA 612 | 612 | CCGGCAAGGACUGCGGCAC | 2663 | GUGCCGCAGUCCUUGCCGG |
| siRNA 616 | 616 | CAAGGACUGCGGCACAGAG | 2667 | CUCUGUGCCGCAGUCCUUG |
| siRNA 624 | 624 | GCGGCACAGAGAAAUGCUU | 2675 | AAGCAUUUCUCUGUGCCGC |
| siRNA 628 | 628 | CACAGAGAAAUGCUUUGAU | 2679 | AUCAAAGCAUUUCUCUGUG |
| siRNA 729 | 729 | GGGGCCGGACCUGGUGCGA | 2780 | UCGCACCAGGUCCGGCCCC |
| siRNA 730 | 730 | GGGCCGGACCUGGUGCGAA | 2781 | UUCGCACCAGGUCCGGCCC |
| siRNA 732 | 732 | GCCGGACCUGGUGCGAAGG | 2783 | CCUUCGCACCAGGUCCGGC |
| siRNA 733 | 733 | CCGGACCUGGUGCGAAGGC | 2784 | GCCUUCGCACCAGGUCCGG |
| siRNA 735 | 735 | GGACCUGGUGCGAAGGCAC | 2786 | GUGCCUUCGCACCAGGUCC |
| siRNA 736 | 736 | GACCUGGUGCGAAGGCACC | 2787 | GGUGCCUUCGCACCAGGUC |
| siRNA 737 | 737 | ACCUGGUGCGAAGGCACCC | 2788 | GGGUGCCUUCGCACCAGGU |
| siRNA 738 | 738 | CCUGGUGCGAAGGCACCCG | 2789 | CGGGUGCCUUCGCACCAGG |
| siRNA 742 | 742 | GUGCGAAGGCACCCGACAU | 2793 | AUGUCGGGUGCCUUCGCAC |
| siRNA 743 | 743 | UGCGAAGGCACCCGACAUA | 2794 | UAUGUCGGGUGCCUUCGCA |
| siRNA 744 | 744 | GCGAAGGCACCCGACAUAC | 2795 | GUAUGUCGGGUGCCUUCGC |
| siRNA 745 | 745 | CGAAGGCACCCGACAUACA | 2796 | UGUAUGUCGGGUGCCUUCG |
| siRNA 747 | 747 | AAGGCACCCGACAUACAGC | 2798 | GCUGUAUGUCGGGUGCCUU |
| siRNA 748 | 748 | AGGCACCCGACAUACAGCU | 2799 | AGCUGUAUGUCGGGUGCCU |
| siRNA 749 | 749 | GGCACCCGACAUACAGCUU | 2800 | AAGCUGUAUGUCGGGUGCC |
| siRNA 751 | 751 | CACCCGACAUACAGCUUGU | 2802 | ACAAGCUGUAUGUCGGGUG |
| siRNA 753 | 753 | CCCGACAUACAGCUUGUCU | 2804 | AGACAAGCUGUAUGUCGGG |
| siRNA 754 | 754 | CCGACAUACAGCUUGUCUG | 2805 | CAGACAAGCUGUAUGUCGG |
| siRNA 755 | 755 | CGACAUACAGCUUGUCUGA | 2806 | UCAGACAAGCUGUAUGUCG |
| siRNA 756 | 756 | GACAUACAGCUUGUCUGAG | 2807 | CUCAGACAAGCUGUAUGUC |
| siRNA 757 | 757 | ACAUACAGCUUGUCUGAGC | 2808 | GCUCAGACAAGCUGUAUGU |
| siRNA 758 | 758 | CAUACAGCUUGUCUGAGCA | 2809 | UGCUCAGACAAGCUGUAUG |
| siRNA 776 | 776 | AGCCCUUGCCUGAACGGGG | 2827 | CCCCGUUCAGGCAAGGGCU |
| siRNA 796 | 796 | CACCUGCCACCUGAUCGUG | 2847 | CACGAUCAGGUGGCAGGUG |
| siRNA 799 | 799 | CUGCCACCUGAUCGUGGCC | 2850 | GGCCACGAUCAGGUGGCAG |
| siRNA 810 | 810 | UCGUGGCCACCGGGACCAC | 2861 | GUGGUCCCGGUGGCCACGA |
| siRNA 819 | 819 | CCGGGACCACCGUGUGUGC | 2870 | GCACACACGGUGGUCCCGG |
| siRNA 848 | 848 | GGCUUCGCUGGACGGCUCU | 2899 | AGAGCCGUCCAGCGAAGCC |
| siRNA 853 | 853 | CGCUGGACGGCUCUGCAAC | 2904 | GUUGCAGAGCCGUCCAGCG |
| siRNA 855 | 855 | CUGGACGGCUCUGCAACAU | 2906 | AUGUUGCAGAGCCGUCCAG |
| siRNA 857 | 857 | GGACGGCUCUGCAACAUCG | 2908 | CGAUGUUGCAGAGCCGUCC |
| siRNA 858 | 858 | GACGGCUCUGCAACAUCGA | 2909 | UCGAUGUUGCAGAGCCGUC |
| siRNA 859 | 859 | ACGGCUCUGCAACAUCGAG | 2910 | CUCGAUGUUGCAGAGCCGU |
| siRNA 861 | 861 | GGCUCUGCAACAUCGAGCC | 2912 | GGCUCGAUGUUGCAGAGCC |
| siRNA 862 | 862 | GCUCUGCAACAUCGAGCCU | 2913 | AGGCUCGAUGUUGCAGAGC |
| siRNA 863 | 863 | CUCUGCAACAUCGAGCCUG | 2914 | CAGGCUCGAUGUUGCAGAG |
| siRNA 871 | 871 | CAUCGAGCCUGAUGAGCGC | 2922 | GCGCUCAUCAGGCUCGAUG |
| siRNA 872 | 872 | AUCGAGCCUGAUGAGCGCU | 2923 | AGCGCUCAUCAGGCUCGAU |
| siRNA 873 | 873 | UCGAGCCUGAUGAGCGCUG | 2924 | CAGCGCUCAUCAGGCUCGA |
| siRNA 875 | 875 | GAGCCUGAUGAGCGCUGCU | 2926 | AGCAGCGCUCAUCAGGCUC |
| siRNA 879 | 879 | CUGAUGAGCGCUGCUUCUU | 2930 | AAGAAGCAGCGCUCAUCAG |
| siRNA 896 | 896 | UUGGGGAACGGCACUGGGU | 2947 | ACCCAGUGCCGUUCCCCAA |
| siRNA 901 | 901 | GAACGGCACUGGGUACCGU | 2952 | ACGGUACCCAGUGCCGUUC |
| siRNA 902 | 902 | AACGGCACUGGGUACCGUG | 2953 | CACGGUACCCAGUGCCGUU |
| siRNA 908 | 908 | ACUGGGUACCGUGGCGUGG | 2959 | CCACGCCACGGUACCCAGU |
| siRNA 909 | 909 | CUGGGUACCGUGGCGUGGC | 2960 | GCCACGCCACGGUACCCAG |
| siRNA 910 | 910 | UGGGUACCGUGGCGUGGCC | 2961 | GGCCACGCCACGGUACCCA |
| siRNA 911 | 911 | GGGUACCGUGGCGUGGCCA | 2962 | UGGCCACGCCACGGUACCC |
| siRNA 912 | 912 | GGUACCGUGGCGUGGCCAG | 2963 | CUGGCCACGCCACGGUACC |
| siRNA 913 | 913 | GUACCGUGGCGUGGCCAGC | 2964 | GCUGGCCACGCCACGGUAC |
| siRNA 916 | 916 | CCGUGGCGUGGCCAGCACC | 2967 | GGUGCUGGCCACGCCACGG |
| siRNA 957 | 957 | UGGCCUGGAACUCCGAUCU | 3008 | AGAUCGGAGUUCCAGGCCA |
| siRNA 959 | 959 | GCCUGGAACUCCGAUCUGC | 3010 | GCAGAUCGGAGUUCCAGGC |
| siRNA 960 | 960 | CCUGGAACUCCGAUCUGCU | 3011 | AGCAGAUCGGAGUUCCAGG |
| siRNA 961 | 961 | CUGGAACUCCGAUCUGCUC | 3012 | GAGCAGAUCGGAGUUCCAG |
| siRNA 962 | 962 | UGGAACUCCGAUCUGCUCU | 3013 | AGAGCAGAUCGGAGUUCCA |
| siRNA 964 | 964 | GAACUCCGAUCUGCUCUAC | 3015 | GUAGAGCAGAUCGGAGUUC |
| siRNA 965 | 965 | AACUCCGAUCUGCUCUACC | 3016 | GGUAGAGCAGAUCGGAGUU |
| siRNA 992 | 992 | CACGUGGACUCCGUGGGCG | 3043 | CGCCCACGGAGUCCACGUG |
| siRNA 996 | 996 | UGGACUCCGUGGGCGCCGC | 3047 | GCGGCGCCCACGGAGUCCA |
| siRNA 998 | 998 | GACUCCGUGGGCGCCGCGG | 3049 | CCGCGGCGCCCACGGAGUC |
| siRNA 1027 | 1027 | CCUGGGCCCCCAUGCCUAC | 3078 | GUAGGCAUGGGGGCCCAGG |
| siRNA 1028 | 1028 | CUGGGCCCCCAUGCCUACU | 3079 | AGUAGGCAUGGGGGCCCAG |
| siRNA 1033 | 1033 | CCCCCAUGCCUACUGCCGG | 3084 | CCGGCAGUAGGCAUGGGGG |
| siRNA 1034 | 1034 | CCCCAUGCCUACUGCCGGA | 3085 | UCCGGCAGUAGGCAUGGGG |
| siRNA 1035 | 1035 | CCCAUGCCUACUGCCGGAA | 3086 | UUCCGGCAGUAGGCAUGGG |
| siRNA 1039 | 1039 | UGCCUACUGCCGGAAUCCG | 3090 | CGGAUUCCGGCAGUAGGCA |
| siRNA 1040 | 1040 | GCCUACUGCCGGAAUCCGG | 3091 | CCGGAUUCCGGCAGUAGGC |
| siRNA 1041 | 1041 | CCUACUGCCGGAAUCCGGA | 3092 | UCCGGAUUCCGGCAGUAGG |
| siRNA 1042 | 1042 | CUACUGCCGGAAUCCGGAC | 3093 | GUCCGGAUUCCGGCAGUAG |
| siRNA 1043 | 1043 | UACUGCCGGAAUCCGGACA | 3094 | UGUCCGGAUUCCGGCAGUA |
| siRNA 1047 | 1047 | GCCGGAAUCCGGACAAUGA | 3098 | UCAUUGUCCGGAUUCCGGC |
| siRNA 1048 | 1048 | CCGGAAUCCGGACAAUGAC | 3099 | GUCAUUGUCCGGAUUCCGG |
| siRNA 1050 | 1050 | GGAAUCCGGACAAUGACGA | 3101 | UCGUCAUUGUCCGGAUUCC |
| siRNA 1051 | 1051 | GAAUCCGGACAAUGACGAG | 3102 | CUCGUCAUUGUCCGGAUUC |
| siRNA 1052 | 1052 | AAUCCGGACAAUGACGAGA | 3103 | UCUCGUCAUUGUCCGGAUU |
| siRNA 1053 | 1053 | AUCCGGACAAUGACGAGAG | 3104 | CUCUCGUCAUUGUCCGGAU |
| siRNA 1054 | 1054 | UCCGGACAAUGACGAGAGG | 3105 | CCUCUCGUCAUUGUCCGGA |
| siRNA 1055 | 1055 | CCGGACAAUGACGAGAGGC | 3106 | GCCUCUCGUCAUUGUCCGG |
| siRNA 1067 | 1067 | GAGAGGCCCUGGUGCUACG | 3118 | CGUAGCACCAGGGCCUCUC |
| siRNA 1100 | 1100 | GCGCUCUCCUGGGAGUACU | 3151 | AGUACUCCCAGGAGAGCGC |
| siRNA 1106 | 1106 | UCCUGGGAGUACUGCCGCC | 3157 | GGCGGCAGUACUCCCAGGA |
| siRNA 1121 | 1121 | CGCCUGGAGGCCUGCGAAU | 3172 | AUUCGCAGGCCUCCAGGCG |
| siRNA 1122 | 1122 | GCCUGGAGGCCUGCGAAUC | 3173 | GAUUCGCAGGCCUCCAGGC |
| siRNA 1123 | 1123 | CCUGGAGGCCUGCGAAUCC | 3174 | GGAUUCGCAGGCCUCCAGG |
| siRNA 1131 | 1131 | CCUGCGAAUCCCUCACCAG | 3182 | CUGGUGAGGGAUUCGCAGG |
| siRNA 1133 | 1133 | UGCGAAUCCCUCACCAGAG | 3184 | CUCUGGUGAGGGAUUCGCA |
| siRNA 1136 | 1136 | GAAUCCCUCACCAGAGUCC | 3187 | GGACUCUGGUGAGGGAUUC |
| siRNA 1141 | 1141 | CCUCACCAGAGUCCAACUG | 3192 | CAGUUGGACUCUGGUGAGG |
| siRNA 1143 | 1143 | UCACCAGAGUCCAACUGUC | 3194 | GACAGUUGGACUCUGGUGA |
| siRNA 1144 | 1144 | CACCAGAGUCCAACUGUCA | 3195 | UGACAGUUGGACUCUGGUG |
| siRNA 1149 | 1149 | GAGUCCAACUGUCACCGGA | 3200 | UCCGGUGACAGUUGGACUC |
| siRNA 1151 | 1151 | GUCCAACUGUCACCGGAUC | 3202 | GAUCCGGUGACAGUUGGAC |
| siRNA 1152 | 1152 | UCCAACUGUCACCGGAUCU | 3203 | AGAUCCGGUGACAGUUGGA |
| siRNA 1153 | 1153 | CCAACUGUCACCGGAUCUC | 3204 | GAGAUCCGGUGACAGUUGG |
| siRNA 1161 | 1161 | CACCGGAUCUCCUGGCGAC | 3212 | GUCGCCAGGAGAUCCGGUG |
| siRNA 1166 | 1166 | GAUCUCCUGGCGACCCUGC | 3217 | GCAGGGUCGCCAGGAGAUC |
| siRNA 1203 | 1203 | GGCGCCAGGCCUGCGGCAG | 3254 | CUGCCGCAGGCCUGGCGCC |
| siRNA 1215 | 1215 | GCGGCAGGAGGCACAAGAA | 3266 | UUCUUGUGCCUCCUGCCGC |
| siRNA 1224 | 1224 | GGCACAAGAAGAGGACGUU | 3275 | AACGUCCUCUUCUUGUGCC |
| siRNA 1225 | 1225 | GCACAAGAAGAGGACGUUC | 3276 | GAACGUCCUCUUCUUGUGC |
| siRNA 1226 | 1226 | CACAAGAAGAGGACGUUCC | 3277 | GGAACGUCCUCUUCUUGUG |
| siRNA 1235 | 1235 | AGGACGUUCCUGCGGCCAC | 3286 | GUGGCCGCAGGAACGUCCU |
| siRNA 1236 | 1236 | GGACGUUCCUGCGGCCACG | 3287 | CGUGGCCGCAGGAACGUCC |
| siRNA 1238 | 1238 | ACGUUCCUGCGGCCACGUA | 3289 | UACGUGGCCGCAGGAACGU |
| siRNA 1240 | 1240 | GUUCCUGCGGCCACGUAUC | 3291 | GAUACGUGGCCGCAGGAAC |
| siRNA 1242 | 1242 | UCCUGCGGCCACGUAUCAU | 3293 | AUGAUACGUGGCCGCAGGA |
| siRNA 1243 | 1243 | CCUGCGGCCACGUAUCAUC | 3294 | GAUGAUACGUGGCCGCAGG |
| siRNA 1244 | 1244 | CUGCGGCCACGUAUCAUCG | 3295 | CGAUGAUACGUGGCCGCAG |
| siRNA 1246 | 1246 | GCGGCCACGUAUCAUCGGC | 3297 | GCCGAUGAUACGUGGCCGC |
| siRNA 1247 | 1247 | CGGCCACGUAUCAUCGGCG | 3298 | CGCCGAUGAUACGUGGCCG |
| siRNA 1248 | 1248 | GGCCACGUAUCAUCGGCGG | 3299 | CCGCCGAUGAUACGUGGCC |
| siRNA 1249 | 1249 | GCCACGUAUCAUCGGCGGC | 3300 | GCCGCCGAUGAUACGUGGC |
| siRNA 1250 | 1250 | CCACGUAUCAUCGGCGGCU | 3301 | AGCCGCCGAUGAUACGUGG |
| siRNA 1251 | 1251 | CACGUAUCAUCGGCGGCUC | 3302 | GAGCCGCCGAUGAUACGUG |
| siRNA 1274 | 1274 | UCGCUGCCCGGCUCGCACC | 3325 | GGUGCGAGCCGGGCAGCGA |
| siRNA 1299 | 1299 | UGGCCGCCAUCUACAUCGG | 3350 | CCGAUGUAGAUGGCGGCCA |
| siRNA 1303 | 1303 | CGCCAUCUACAUCGGGGAC | 3354 | GUCCCCGAUGUAGAUGGCG |
| siRNA 1307 | 1307 | AUCUACAUCGGGGACAGCU | 3358 | AGCUGUCCCCGAUGUAGAU |
| siRNA 1309 | 1309 | CUACAUCGGGGACAGCUUC | 3360 | GAAGCUGUCCCCGAUGUAG |
| siRNA 1310 | 1310 | UACAUCGGGGACAGCUUCU | 3361 | AGAAGCUGUCCCCGAUGUA |
| siRNA 1312 | 1312 | CAUCGGGGACAGCUUCUGC | 3363 | GCAGAAGCUGUCCCCGAUG |
| siRNA 1314 | 1314 | UCGGGGACAGCUUCUGCGC | 3365 | GCGCAGAAGCUGUCCCCGA |
| siRNA 1315 | 1315 | CGGGGACAGCUUCUGCGCC | 3366 | GGCGCAGAAGCUGUCCCCG |
| siRNA 1318 | 1318 | GGACAGCUUCUGCGCCGGG | 3369 | CCCGGCGCAGAAGCUGUCC |
| siRNA 1319 | 1319 | GACAGCUUCUGCGCCGGGA | 3370 | UCCCGGCGCAGAAGCUGUC |
| siRNA 1352 | 1352 | UGCUGGGUGGUGUCGGCCG | 3403 | CGGCCGACACCACCCAGCA |
| siRNA 1353 | 1353 | GCUGGGUGGUGUCGGCCGC | 3404 | GCGGCCGACACCACCCAGC |
| siRNA 1379 | 1379 | UUCUCCCACAGCCCCCCCA | 3430 | UGGGGGGGCUGUGGGAGAA |
| siRNA 1389 | 1389 | GCCCCCCCAGGGACAGCGU | 3440 | ACGCUGUCCCUGGGGGGGC |
| siRNA 1390 | 1390 | CCCCCCCAGGGACAGCGUC | 3441 | GACGCUGUCCCUGGGGGGG |
| siRNA 1392 | 1392 | CCCCCAGGGACAGCGUCUC | 3443 | GAGACGCUGUCCCUGGGGG |
| siRNA 1424 | 1424 | CAGCACUUCUUCAACCGCA | 3475 | UGCGGUUGAAGAAGUGCUG |
| siRNA 1427 | 1427 | CACUUCUUCAACCGCACGA | 3478 | UCGUGCGGUUGAAGAAGUG |
| siRNA 1428 | 1428 | ACUUCUUCAACCGCACGAC | 3479 | GUCGUGCGGUUGAAGAAGU |
| siRNA 1429 | 1429 | CUUCUUCAACCGCACGACG | 3480 | CGUCGUGCGGUUGAAGAAG |
| siRNA 1430 | 1430 | UUCUUCAACCGCACGACGG | 3481 | CCGUCGUGCGGUUGAAGAA |
| siRNA 1431 | 1431 | UCUUCAACCGCACGACGGA | 3482 | UCCGUCGUGCGGUUGAAGA |
| siRNA 1432 | 1432 | CUUCAACCGCACGACGGAC | 3483 | GUCCGUCGUGCGGUUGAAG |
| siRNA 1433 | 1433 | UUCAACCGCACGACGGACG | 3484 | CGUCCGUCGUGCGGUUGAA |
| siRNA 1434 | 1434 | UCAACCGCACGACGGACGU | 3485 | ACGUCCGUCGUGCGGUUGA |
| siRNA 1435 | 1435 | CAACCGCACGACGGACGUG | 3486 | CACGUCCGUCGUGCGGUUG |
| siRNA 1436 | 1436 | AACCGCACGACGGACGUGA | 3487 | UCACGUCCGUCGUGCGGUU |
| siRNA 1438 | 1438 | CCGCACGACGGACGUGACG | 3489 | CGUCACGUCCGUCGUGCGG |
| siRNA 1439 | 1439 | CGCACGACGGACGUGACGC | 3490 | GCGUCACGUCCGUCGUGCG |
| siRNA 1440 | 1440 | GCACGACGGACGUGACGCA | 3491 | UGCGUCACGUCCGUCGUGC |
| siRNA 1441 | 1441 | CACGACGGACGUGACGCAG | 3492 | CUGCGUCACGUCCGUCGUG |
| siRNA 1442 | 1442 | ACGACGGACGUGACGCAGA | 3493 | UCUGCGUCACGUCCGUCGU |
| siRNA 1443 | 1443 | CGACGGACGUGACGCAGAC | 3494 | GUCUGCGUCACGUCCGUCG |
| siRNA 1445 | 1445 | ACGGACGUGACGCAGACCU | 3496 | AGGUCUGCGUCACGUCCGU |
| siRNA 1446 | 1446 | CGGACGUGACGCAGACCUU | 3497 | AAGGUCUGCGUCACGUCCG |
| siRNA 1447 | 1447 | GGACGUGACGCAGACCUUC | 3498 | GAAGGUCUGCGUCACGUCC |
| siRNA 1449 | 1449 | ACGUGACGCAGACCUUCGG | 3500 | CCGAAGGUCUGCGUCACGU |
| siRNA 1452 | 1452 | UGACGCAGACCUUCGGCAU | 3503 | AUGCCGAAGGUCUGCGUCA |
| siRNA 1453 | 1453 | GACGCAGACCUUCGGCAUC | 3504 | GAUGCCGAAGGUCUGCGUC |
| siRNA 1455 | 1455 | CGCAGACCUUCGGCAUCGA | 3506 | UCGAUGCCGAAGGUCUGCG |
| siRNA 1456 | 1456 | GCAGACCUUCGGCAUCGAG | 3507 | CUCGAUGCCGAAGGUCUGC |
| siRNA 1459 | 1459 | GACCUUCGGCAUCGAGAAG | 3510 | CUUCUCGAUGCCGAAGGUC |
| siRNA 1460 | 1460 | ACCUUCGGCAUCGAGAAGU | 3511 | ACUUCUCGAUGCCGAAGGU |
| siRNA 1463 | 1463 | UUCGGCAUCGAGAAGUACA | 3514 | UGUACUUCUCGAUGCCGAA |
| siRNA 1464 | 1464 | UCGGCAUCGAGAAGUACAU | 3515 | AUGUACUUCUCGAUGCCGA |
| siRNA 1469 | 1469 | AUCGAGAAGUACAUCCCGU | 3520 | ACGGGAUGUACUUCUCGAU |
| siRNA 1470 | 1470 | UCGAGAAGUACAUCCCGUA | 3521 | UACGGGAUGUACUUCUCGA |
| siRNA 1471 | 1471 | CGAGAAGUACAUCCCGUAC | 3522 | GUACGGGAUGUACUUCUCG |
| siRNA 1474 | 1474 | GAAGUACAUCCCGUACACC | 3525 | GGUGUACGGGAUGUACUUC |
| siRNA 1475 | 1475 | AAGUACAUCCCGUACACCC | 3526 | GGGUGUACGGGAUGUACUU |
| siRNA 1476 | 1476 | AGUACAUCCCGUACACCCU | 3527 | AGGGUGUACGGGAUGUACU |
| siRNA 1477 | 1477 | GUACAUCCCGUACACCCUG | 3528 | CAGGGUGUACGGGAUGUAC |
| siRNA 1478 | 1478 | UACAUCCCGUACACCCUGU | 3529 | ACAGGGUGUACGGGAUGUA |
| siRNA 1504 | 1504 | GUUCAACCCCAGCGACCAC | 3555 | GUGGUCGCUGGGGUUGAAC |
| siRNA 1506 | 1506 | UCAACCCCAGCGACCACGA | 3557 | UCGUGGUCGCUGGGGUUGA |
| siRNA 1507 | 1507 | CAACCCCAGCGACCACGAC | 3558 | GUCGUGGUCGCUGGGGUUG |
| siRNA 1509 | 1509 | ACCCCAGCGACCACGACCU | 3560 | AGGUCGUGGUCGCUGGGGU |
| siRNA 1512 | 1512 | CCAGCGACCACGACCUCGU | 3563 | ACGAGGUCGUGGUCGCUGG |
| siRNA 1513 | 1513 | CAGCGACCACGACCUCGUC | 3564 | GACGAGGUCGUGGUCGCUG |
| siRNA 1516 | 1516 | CGACCACGACCUCGUCCUG | 3567 | CAGGACGAGGUCGUGGUCG |
| siRNA 1518 | 1518 | ACCACGACCUCGUCCUGAU | 3569 | AUCAGGACGAGGUCGUGGU |
| siRNA 1519 | 1519 | CCACGACCUCGUCCUGAUC | 3570 | GAUCAGGACGAGGUCGUGG |
| siRNA 1521 | 1521 | ACGACCUCGUCCUGAUCCG | 3572 | CGGAUCAGGACGAGGUCGU |
| siRNA 1523 | 1523 | GACCUCGUCCUGAUCCGGC | 3574 | GCCGGAUCAGGACGAGGUC |
| siRNA 1524 | 1524 | ACCUCGUCCUGAUCCGGCU | 3575 | AGCCGGAUCAGGACGAGGU |
| siRNA 1528 | 1528 | CGUCCUGAUCCGGCUGAAG | 3579 | CUUCAGCCGGAUCAGGACG |
| siRNA 1562 | 1562 | UGUGCCACACGCUCGCAGU | 3613 | ACUGCGAGCGUGUGGCACA |
| siRNA 1563 | 1563 | GUGCCACACGCUCGCAGUU | 3614 | AACUGCGAGCGUGUGGCAC |
| siRNA 1564 | 1564 | UGCCACACGCUCGCAGUUC | 3615 | GAACUGCGAGCGUGUGGCA |
| siRNA 1565 | 1565 | GCCACACGCUCGCAGUUCG | 3616 | CGAACUGCGAGCGUGUGGC |
| siRNA 1566 | 1566 | CCACACGCUCGCAGUUCGU | 3617 | ACGAACUGCGAGCGUGUGG |
| siRNA 1567 | 1567 | CACACGCUCGCAGUUCGUG | 3618 | CACGAACUGCGAGCGUGUG |
| siRNA 1568 | 1568 | ACACGCUCGCAGUUCGUGC | 3619 | GCACGAACUGCGAGCGUGU |
| siRNA 1572 | 1572 | GCUCGCAGUUCGUGCAGCC | 3623 | GGCUGCACGAACUGCGAGC |
| siRNA 1574 | 1574 | UCGCAGUUCGUGCAGCCCA | 3625 | UGGGCUGCACGAACUGCGA |
| siRNA 1615 | 1615 | CACCUUCCCCGCAGGACAC | 3666 | GUGUCCUGCGGGGAAGGUG |
| siRNA 1616 | 1616 | ACCUUCCCCGCAGGACACA | 3667 | UGUGUCCUGCGGGGAAGGU |
| siRNA 1617 | 1617 | CCUUCCCCGCAGGACACAA | 3668 | UUGUGUCCUGCGGGGAAGG |
| siRNA 1619 | 1619 | UUCCCCGCAGGACACAAGU | 3670 | ACUUGUGUCCUGCGGGGAA |
| siRNA 1621 | 1621 | CCCCGCAGGACACAAGUGC | 3672 | GCACUUGUGUCCUGCGGGG |
| siRNA 1622 | 1622 | CCCGCAGGACACAAGUGCC | 3673 | GGCACUUGUGUCCUGCGGG |
| siRNA 1623 | 1623 | CCGCAGGACACAAGUGCCA | 3674 | UGGCACUUGUGUCCUGCGG |
| siRNA 1624 | 1624 | CGCAGGACACAAGUGCCAG | 3675 | CUGGCACUUGUGUCCUGCG |
| siRNA 1628 | 1628 | GGACACAAGUGCCAGAUUG | 3679 | CAAUCUGGCACUUGUGUCC |
| siRNA 1630 | 1630 | ACACAAGUGCCAGAUUGCG | 3681 | CGCAAUCUGGCACUUGUGU |
| siRNA 1631 | 1631 | CACAAGUGCCAGAUUGCGG | 3682 | CCGCAAUCUGGCACUUGUG |
| siRNA 1635 | 1635 | AGUGCCAGAUUGCGGGCUG | 3686 | CAGCCCGCAAUCUGGCACU |
| siRNA 1636 | 1636 | GUGCCAGAUUGCGGGCUGG | 3687 | CCAGCCCGCAAUCUGGCAC |
| siRNA 1637 | 1637 | UGCCAGAUUGCGGGCUGGG | 3688 | CCCAGCCCGCAAUCUGGCA |
| siRNA 1643 | 1643 | AUUGCGGGCUGGGGCCACU | 3694 | AGUGGCCCCAGCCCGCAAU |
| siRNA 1651 | 1651 | CUGGGGCCACUUGGAUGAG | 3702 | CUCAUCCAAGUGGCCCCAG |
| siRNA 1662 | 1662 | UGGAUGAGAACGUGAGCGG | 3713 | CCGCUCACGUUCUCAUCCA |
| siRNA 1663 | 1663 | GGAUGAGAACGUGAGCGGC | 3714 | GCCGCUCACGUUCUCAUCC |
| siRNA 1664 | 1664 | GAUGAGAACGUGAGCGGCU | 3715 | AGCCGCUCACGUUCUCAUC |
| siRNA 1672 | 1672 | CGUGAGCGGCUACUCCAGC | 3723 | GCUGGAGUAGCCGCUCACG |
| siRNA 1707 | 1707 | UGGUCCCCCUGGUCGCCGA | 3758 | UCGGCGACCAGGGGGACCA |
| siRNA 1708 | 1708 | GGUCCCCCUGGUCGCCGAC | 3759 | GUCGGCGACCAGGGGGACC |
| siRNA 1709 | 1709 | GUCCCCCUGGUCGCCGACC | 3760 | GGUCGGCGACCAGGGGGAC |
| siRNA 1712 | 1712 | CCCCUGGUCGCCGACCACA | 3763 | UGUGGUCGGCGACCAGGGG |
| siRNA 1713 | 1713 | CCCUGGUCGCCGACCACAA | 3764 | UUGUGGUCGGCGACCAGGG |
| siRNA 1715 | 1715 | CUGGUCGCCGACCACAAGU | 3766 | ACUUGUGGUCGGCGACCAG |
| siRNA 1716 | 1716 | UGGUCGCCGACCACAAGUG | 3767 | CACUUGUGGUCGGCGACCA |
| siRNA 1717 | 1717 | GGUCGCCGACCACAAGUGC | 3768 | GCACUUGUGGUCGGCGACC |
| siRNA 1718 | 1718 | GUCGCCGACCACAAGUGCA | 3769 | UGCACUUGUGGUCGGCGAC |
| siRNA 1722 | 1722 | CCGACCACAAGUGCAGCAG | 3773 | CUGCUGCACUUGUGGUCGG |
| siRNA 1737 | 1737 | GCAGCCCUGAGGUCUACGG | 3788 | CCGUAGACCUCAGGGCUGC |
| siRNA 1739 | 1739 | AGCCCUGAGGUCUACGGCG | 3790 | CGCCGUAGACCUCAGGGCU |
| siRNA 1741 | 1741 | CCCUGAGGUCUACGGCGCC | 3792 | GGCGCCGUAGACCUCAGGG |
| siRNA 1743 | 1743 | CUGAGGUCUACGGCGCCGA | 3794 | UCGGCGCCGUAGACCUCAG |
| siRNA 1745 | 1745 | GAGGUCUACGGCGCCGACA | 3796 | UGUCGGCGCCGUAGACCUC |
| siRNA 1749 | 1749 | UCUACGGCGCCGACAUCAG | 3800 | CUGAUGUCGGCGCCGUAGA |
| siRNA 1751 | 1751 | UACGGCGCCGACAUCAGCC | 3802 | GGCUGAUGUCGGCGCCGUA |
| siRNA 1752 | 1752 | ACGGCGCCGACAUCAGCCC | 3803 | GGGCUGAUGUCGGCGCCGU |
| siRNA 1753 | 1753 | CGGCGCCGACAUCAGCCCC | 3804 | GGGGCUGAUGUCGGCGCCG |
| siRNA 1759 | 1759 | CGACAUCAGCCCCAACAUG | 3810 | CAUGUUGGGGCUGAUGUCG |
| siRNA 1766 | 1766 | AGCCCCAACAUGCUCUGUG | 3817 | CACAGAGCAUGUUGGGGCU |
| siRNA 1770 | 1770 | CCAACAUGCUCUGUGCCGG | 3821 | CCGGCACAGAGCAUGUUGG |
| siRNA 1780 | 1780 | CUGUGCCGGCUACUUCGAC | 3831 | GUCGAAGUAGCCGGCACAG |
| siRNA 1781 | 1781 | UGUGCCGGCUACUUCGACU | 3832 | AGUCGAAGUAGCCGGCACA |
| siRNA 1782 | 1782 | GUGCCGGCUACUUCGACUG | 3833 | CAGUCGAAGUAGCCGGCAC |
| siRNA 1783 | 1783 | UGCCGGCUACUUCGACUGC | 3834 | GCAGUCGAAGUAGCCGGCA |
| siRNA 1785 | 1785 | CCGGCUACUUCGACUGCAA | 3836 | UUGCAGUCGAAGUAGCCGG |
| siRNA 1786 | 1786 | CGGCUACUUCGACUGCAAG | 3837 | CUUGCAGUCGAAGUAGCCG |
| siRNA 1788 | 1788 | GCUACUUCGACUGCAAGUC | 3839 | GACUUGCAGUCGAAGUAGC |
| siRNA 1789 | 1789 | CUACUUCGACUGCAAGUCC | 3840 | GGACUUGCAGUCGAAGUAG |
| siRNA 1791 | 1791 | ACUUCGACUGCAAGUCCGA | 3842 | UCGGACUUGCAGUCGAAGU |
| siRNA 1792 | 1792 | CUUCGACUGCAAGUCCGAC | 3843 | GUCGGACUUGCAGUCGAAG |
| siRNA 1793 | 1793 | UUCGACUGCAAGUCCGACG | 3844 | CGUCGGACUUGCAGUCGAA |
| siRNA 1794 | 1794 | UCGACUGCAAGUCCGACGC | 3845 | GCGUCGGACUUGCAGUCGA |
| siRNA 1795 | 1795 | CGACUGCAAGUCCGACGCC | 3846 | GGCGUCGGACUUGCAGUCG |
| siRNA 1797 | 1797 | ACUGCAAGUCCGACGCCUG | 3848 | CAGGCGUCGGACUUGCAGU |
| siRNA 1798 | 1798 | CUGCAAGUCCGACGCCUGC | 3849 | GCAGGCGUCGGACUUGCAG |
| siRNA 1799 | 1799 | UGCAAGUCCGACGCCUGCC | 3850 | GGCAGGCGUCGGACUUGCA |
| siRNA 1840 | 1840 | GGCCUGCGAGAAGAACGGC | 3891 | GCCGUUCUUCUCGCAGGCC |
| siRNA 1843 | 1843 | CUGCGAGAAGAACGGCGUG | 3894 | CACGCCGUUCUUCUCGCAG |
| siRNA 1849 | 1849 | GAAGAACGGCGUGGCUUAC | 3900 | GUAAGCCACGCCGUUCUUC |
| siRNA 1850 | 1850 | AAGAACGGCGUGGCUUACC | 3901 | GGUAAGCCACGCCGUUCUU |
| siRNA 1851 | 1851 | AGAACGGCGUGGCUUACCU | 3902 | AGGUAAGCCACGCCGUUCU |
| siRNA 1856 | 1856 | GGCGUGGCUUACCUCUACG | 3907 | CGUAGAGGUAAGCCACGCC |
| siRNA 1857 | 1857 | GCGUGGCUUACCUCUACGG | 3908 | CCGUAGAGGUAAGCCACGC |
| siRNA 1858 | 1858 | CGUGGCUUACCUCUACGGC | 3909 | GCCGUAGAGGUAAGCCACG |
| siRNA 1859 | 1859 | GUGGCUUACCUCUACGGCA | 3910 | UGCCGUAGAGGUAAGCCAC |
| siRNA 1860 | 1860 | UGGCUUACCUCUACGGCAU | 3911 | AUGCCGUAGAGGUAAGCCA |
| siRNA 1861 | 1861 | GGCUUACCUCUACGGCAUC | 3912 | GAUGCCGUAGAGGUAAGCC |
| siRNA 1862 | 1862 | GCUUACCUCUACGGCAUCA | 3913 | UGAUGCCGUAGAGGUAAGC |
| siRNA 1863 | 1863 | CUUACCUCUACGGCAUCAU | 3914 | AUGAUGCCGUAGAGGUAAG |
| siRNA 1864 | 1864 | UUACCUCUACGGCAUCAUC | 3915 | GAUGAUGCCGUAGAGGUAA |
| siRNA 1895 | 1895 | GGCUGCGGGCGGCUCCACA | 3946 | UGUGGAGCCGCCCGCAGCC |
| siRNA 1896 | 1896 | GCUGCGGGCGGCUCCACAA | 3947 | UUGUGGAGCCGCCCGCAGC |
| siRNA 1897 | 1897 | CUGCGGGCGGCUCCACAAG | 3948 | CUUGUGGAGCCGCCCGCAG |
| siRNA 1900 | 1900 | CGGGCGGCUCCACAAGCCG | 3951 | CGGCUUGUGGAGCCGCCCG |
| siRNA 1901 | 1901 | GGGCGGCUCCACAAGCCGG | 3952 | CCGGCUUGUGGAGCCGCCC |
| siRNA 1902 | 1902 | GGCGGCUCCACAAGCCGGG | 3953 | CCCGGCUUGUGGAGCCGCC |
| siRNA 1903 | 1903 | GCGGCUCCACAAGCCGGGG | 3954 | CCCCGGCUUGUGGAGCCGC |
| siRNA 1904 | 1904 | CGGCUCCACAAGCCGGGGG | 3955 | CCCCCGGCUUGUGGAGCCG |
| siRNA 1906 | 1906 | GCUCCACAAGCCGGGGGUC | 3957 | GACCCCCGGCUUGUGGAGC |
| siRNA 1908 | 1908 | UCCACAAGCCGGGGGUCUA | 3959 | UAGACCCCCGGCUUGUGGA |
| siRNA 1909 | 1909 | CCACAAGCCGGGGGUCUAC | 3960 | GUAGACCCCCGGCUUGUGG |
| siRNA 1910 | 1910 | CACAAGCCGGGGGUCUACA | 3961 | UGUAGACCCCCGGCUUGUG |
| siRNA 1917 | 1917 | CGGGGGUCUACACCCGCGU | 3968 | ACGCGGGUGUAGACCCCCG |
| siRNA 1927 | 1927 | CACCCGCGUGGCCAACUAU | 3978 | AUAGUUGGCCACGCGGGUG |
| siRNA 1928 | 1928 | ACCCGCGUGGCCAACUAUG | 3979 | CAUAGUUGGCCACGCGGGU |
| siRNA 1929 | 1929 | CCCGCGUGGCCAACUAUGU | 3980 | ACAUAGUUGGCCACGCGGG |
| siRNA 1937 | 1937 | GCCAACUAUGUGGACUGGA | 3988 | UCCAGUCCACAUAGUUGGC |
| siRNA 1966 | 1966 | GAUACGGCCUCCCAGGCGG | 4017 | CCGCCUGGGAGGCCGUAUC |
| siRNA 1967 | 1967 | AUACGGCCUCCCAGGCGGC | 4018 | GCCGCCUGGGAGGCCGUAU |
| siRNA 1974 | 1974 | CUCCCAGGCGGCUUGUGGC | 4025 | GCCACAAGCCGCCUGGGAG |
| siRNA 1979 | 1979 | AGGCGGCUUGUGGCUCCCU | 4030 | AGGGAGCCACAAGCCGCCU |
| siRNA 1999 | 1999 | CUGACCCUCCAGCGGGACA | 4050 | UGUCCCGCUGGAGGGUCAG |
| siRNA 2001 | 2001 | GACCCUCCAGCGGGACACC | 4052 | GGUGUCCCGCUGGAGGGUC |
| siRNA 2010 | 2010 | GCGGGACACCCUGGUUCCC | 4061 | GGGAACCAGGGUGUCCCGC |
| siRNA 1999 | 1999 | CUGACCCUCCAGCGGGACA | 4050 | UGUCCCGCUGGAGGGUCAG |
| siRNA 2001 | 2001 | GACCCUCCAGCGGGACACC | 4052 | GGUGUCCCGCUGGAGGGUC |
| siRNA 2010 | 2010 | GCGGGACACCCUGGUUCCC | 4061 | GGGAACCAGGGUGUCCCGC |

The siRNAs in subset B had the following characteristics: Cross-reactivity: With 19mer in human HGFAC mRNA; Specificity category: For human: AS2 or better, SS3 or better; miRNA seeds: AS+SS strand: seed region not conserved in human, mouse, and rat and not present in >4 species; Off-target frequency: <30 human off-targets matched with 2 mismatches in antisense strand; and SNPs: siRNA target sites do not harbor SNPs with a MAF≥1% (pos. 2-18).

The siRNA sequences in subset B were further selected for absence of seed regions in the AS strand that are identical to a seed region of known human miRNA to yield subset C. Subset C includes 248 siRNAs whose base sequences are shown in Table C.

TABLE C
Subset C

| | SEQ ID | sense strand | SEQ ID | antisense strand |
|---|---|---|---|---|
| siRNA NO: | NO: | sequence (5′-3′) | NO: | sequence (5′-3′) |
| siRNA 26 | 26 | UCAGGAGCCAUGGGGCGCU | 2077 | AGCGCCCCAUGGCUCCUGA |
| siRNA 116 | 116 | CUGCUGCCACGGGGGUUCC | 2167 | GGAACCCCCGUGGCAGCAG |
| siRNA 118 | 118 | GCUGCCACGGGGGUUCCAG | 2169 | CUGGAACCCCCGUGGCAGC |
| siRNA 136 | 136 | GCCCCAGCCUGGCGGGAAC | 2187 | GUUCCCGCCAGGCUGGGGC |
| siRNA 140 | 140 | CAGCCUGGCGGGAACCGUA | 2191 | UACGGUUCCCGCCAGGCUG |
| siRNA 141 | 141 | AGCCUGGCGGGAACCGUAC | 2192 | GUACGGUUCCCGCCAGGCU |
| siRNA 143 | 143 | CCUGGCGGGAACCGUACGG | 2194 | CCGUACGGUUCCCGCCAGG |
| siRNA 145 | 145 | UGGCGGGAACCGUACGGAG | 2196 | CUCCGUACGGUUCCCGCCA |
| siRNA 149 | 149 | GGGAACCGUACGGAGUCCC | 2200 | GGGACUCCGUACGGUUCCC |
| siRNA 155 | 155 | CGUACGGAGUCCCCAGAAC | 2206 | GUUCUGGGGACUCCGUACG |
| siRNA 158 | 158 | ACGGAGUCCCCAGAACCUA | 2209 | UAGGUUCUGGGGACUCCGU |
| siRNA 172 | 172 | ACCUAAUGCCACAGCGACC | 2223 | GGUCGCUGUGGCAUUAGGU |
| siRNA 202 | 202 | CACUAUCCUGGUGACCUCU | 2253 | AGAGGUCACCAGGAUAGUG |
| siRNA 232 | 232 | GACCCCAGCAACAAGUGCU | 2283 | AGCACUUGUUGCUGGGGUC |
| siRNA 294 | 294 | CCAGGGCAGUUCCCUCGAG | 2345 | CUCGAGGGAACUGCCCUGG |
| siRNA 295 | 295 | CAGGGCAGUUCCCUCGAGC | 2346 | GCUCGAGGGAACUGCCCUG |
| siRNA 296 | 296 | AGGGCAGUUCCCUCGAGCA | 2347 | UGCUCGAGGGAACUGCCCU |
| siRNA 301 | 301 | AGUUCCCUCGAGCAGUAGC | 2352 | GCUACUGCUCGAGGGAACU |
| siRNA 303 | 303 | UUCCCUCGAGCAGUAGCCC | 2354 | GGGCUACUGCUCGAGGGAA |
| siRNA 328 | 328 | CCAAGCACUCACCGAGGAC | 2379 | GUCCUCGGUGAGUGCUUGG |
| siRNA 334 | 334 | ACUCACCGAGGACGGGAGG | 2385 | CCUCCCGUCCUCGGUGAGU |
| siRNA 335 | 335 | CUCACCGAGGACGGGAGGC | 2386 | GCCUCCCGUCCUCGGUGAG |
| siRNA 360 | 360 | GGUUCCCCUUCCGCUACGG | 2411 | CCGUAGCGGAAGGGGAACC |
| siRNA 361 | 361 | GUUCCCCUUCCGCUACGGG | 2412 | CCCGUAGCGGAAGGGGAAC |
| siRNA 362 | 362 | UUCCCCUUCCGCUACGGGG | 2413 | CCCCGUAGCGGAAGGGGAA |
| siRNA 363 | 363 | UCCCCUUCCGCUACGGGGG | 2414 | CCCCCGUAGCGGAAGGGGA |
| siRNA 371 | 371 | CGCUACGGGGGCCGCAUGC | 2422 | GCAUGCGGCCCCCGUAGCG |
| siRNA 372 | 372 | GCUACGGGGGCCGCAUGCU | 2423 | AGCAUGCGGCCCCCGUAGC |
| siRNA 373 | 373 | CUACGGGGGCCGCAUGCUG | 2424 | CAGCAUGCGGCCCCCGUAG |
| siRNA 378 | 378 | GGGGCCGCAUGCUGCAUGC | 2429 | GCAUGCAGCAUGCGGCCCC |
| siRNA 389 | 389 | CUGCAUGCCUGCACUUCGG | 2440 | CCGAAGUGCAGGCAUGCAG |
| siRNA 391 | 391 | GCAUGCCUGCACUUCGGAG | 2442 | CUCCGAAGUGCAGGCAUGC |
| siRNA 398 | 398 | UGCACUUCGGAGGGCAGUG | 2449 | CACUGCCCUCCGAAGUGCA |
| siRNA 431 | 431 | UGUGCCACAACUCACAACU | 2482 | AGUUGUGAGUUGUGGCACA |
| siRNA 436 | 436 | CACAACUCACAACUACGAC | 2487 | GUCGUAGUUGUGAGUUGUG |
| siRNA 438 | 438 | CAACUCACAACUACGACCG | 2489 | CGGUCGUAGUUGUGAGUUG |
| siRNA 442 | 442 | UCACAACUACGACCGGGAC | 2493 | GUCCCGGUCGUAGUUGUGA |
| siRNA 445 | 445 | CAACUACGACCGGGACAGG | 2496 | CCUGUCCCGGUCGUAGUUG |
| siRNA 473 | 473 | UACUGUGUGGAGGCCACCC | 2524 | GGGUGGCCUCCACACAGUA |
| siRNA 530 | 530 | UCCGGCCCCUGCCUCAAUG | 2581 | CAUUGAGGCAGGGGCCGGA |
| siRNA 531 | 531 | CCGGCCCCUGCCUCAAUGG | 2582 | CCAUUGAGGCAGGGGCCGG |
| siRNA 533 | 533 | GGCCCCUGCCUCAAUGGAG | 2584 | CUCCAUUGAGGCAGGGGCC |
| siRNA 549 | 549 | GAGGCUCCUGCUCCAAUAC | 2600 | GUAUUGGAGCAGGAGCCUC |
| siRNA 558 | 558 | GCUCCAAUACCCAGGACCC | 2609 | GGGUCCUGGGUAUUGGAGC |
| siRNA 564 | 564 | AUACCCAGGACCCCCAGUC | 2615 | GACUGGGGGUCCUGGGUAU |
| siRNA 569 | 569 | CAGGACCCCCAGUCCUAUC | 2620 | GAUAGGACUGGGGGUCCUG |
| siRNA 601 | 601 | CCGGGCCUUCACCGGCAAG | 2652 | CUUGCCGGUGAAGGCCCGG |
| siRNA 602 | 602 | CGGGCCUUCACCGGCAAGG | 2653 | CCUUGCCGGUGAAGGCCCG |
| siRNA 612 | 612 | CCGGCAAGGACUGCGGCAC | 2663 | GUGCCGCAGUCCUUGCCGG |
| siRNA 616 | 616 | CAAGGACUGCGGCACAGAG | 2667 | CUCUGUGCCGCAGUCCUUG |
| siRNA 628 | 628 | CACAGAGAAAUGCUUUGAU | 2679 | AUCAAAGCAUUUCUCUGUG |
| siRNA 729 | 729 | GGGGCCGGACCUGGUGCGA | 2780 | UCGCACCAGGUCCGGCCCC |
| siRNA 730 | 730 | GGGCCGGACCUGGUGCGAA | 2781 | UUCGCACCAGGUCCGGCCC |
| siRNA 732 | 732 | GCCGGACCUGGUGCGAAGG | 2783 | CCUUCGCACCAGGUCCGGC |
| siRNA 733 | 733 | CCGGACCUGGUGCGAAGGC | 2784 | GCCUUCGCACCAGGUCCGG |
| siRNA 735 | 735 | GGACCUGGUGCGAAGGCAC | 2786 | GUGCCUUCGCACCAGGUCC |
| siRNA 738 | 738 | CCUGGUGCGAAGGCACCCG | 2789 | CGGGUGCCUUCGCACCAGG |
| siRNA 742 | 742 | GUGCGAAGGCACCCGACAU | 2793 | AUGUCGGGUGCCUUCGCAC |
| siRNA 743 | 743 | UGCGAAGGCACCCGACAUA | 2794 | UAUGUCGGGUGCCUUCGCA |
| siRNA 744 | 744 | GCGAAGGCACCCGACAUAC | 2795 | GUAUGUCGGGUGCCUUCGC |
| siRNA 749 | 749 | GGCACCCGACAUACAGCUU | 2800 | AAGCUGUAUGUCGGGUGCC |
| siRNA 751 | 751 | CACCCGACAUACAGCUUGU | 2802 | ACAAGCUGUAUGUCGGGUG |
| siRNA 753 | 753 | CCCGACAUACAGCUUGUCU | 2804 | AGACAAGCUGUAUGUCGGG |
| siRNA 754 | 754 | CCGACAUACAGCUUGUCUG | 2805 | CAGACAAGCUGUAUGUCGG |
| siRNA 756 | 756 | GACAUACAGCUUGUCUGAG | 2807 | CUCAGACAAGCUGUAUGUC |
| siRNA 776 | 776 | AGCCCUUGCCUGAACGGGG | 2827 | CCCCGUUCAGGCAAGGGCU |
| siRNA 796 | 796 | CACCUGCCACCUGAUCGUG | 2847 | CACGAUCAGGUGGCAGGUG |
| siRNA 799 | 799 | CUGCCACCUGAUCGUGGCC | 2850 | GGCCACGAUCAGGUGGCAG |
| siRNA 810 | 810 | UCGUGGCCACCGGGACCAC | 2861 | GUGGUCCCGGUGGCCACGA |
| siRNA 819 | 819 | CCGGGACCACCGUGUGUGC | 2870 | GCACACACGGUGGUCCCGG |
| siRNA 857 | 857 | GGACGGCUCUGCAACAUCG | 2908 | CGAUGUUGCAGAGCCGUCC |
| siRNA 858 | 858 | GACGGCUCUGCAACAUCGA | 2909 | UCGAUGUUGCAGAGCCGUC |
| siRNA 859 | 859 | ACGGCUCUGCAACAUCGAG | 2910 | CUCGAUGUUGCAGAGCCGU |
| siRNA 861 | 861 | GGCUCUGCAACAUCGAGCC | 2912 | GGCUCGAUGUUGCAGAGCC |
| siRNA 862 | 862 | GCUCUGCAACAUCGAGCCU | 2913 | AGGCUCGAUGUUGCAGAGC |
| siRNA 863 | 863 | CUCUGCAACAUCGAGCCUG | 2914 | CAGGCUCGAUGUUGCAGAG |
| siRNA 871 | 871 | CAUCGAGCCUGAUGAGCGC | 2922 | GCGCUCAUCAGGCUCGAUG |
| siRNA 872 | 872 | AUCGAGCCUGAUGAGCGCU | 2923 | AGCGCUCAUCAGGCUCGAU |
| siRNA 873 | 873 | UCGAGCCUGAUGAGCGCUG | 2924 | CAGCGCUCAUCAGGCUCGA |
| siRNA 875 | 875 | GAGCCUGAUGAGCGCUGCU | 2926 | AGCAGCGCUCAUCAGGCUC |
| siRNA 879 | 879 | CUGAUGAGCGCUGCUUCUU | 2930 | AAGAAGCAGCGCUCAUCAG |
| siRNA 896 | 896 | UUGGGGAACGGCACUGGGU | 2947 | ACCCAGUGCCGUUCCCCAA |
| siRNA 901 | 901 | GAACGGCACUGGGUACCGU | 2952 | ACGGUACCCAGUGCCGUUC |
| siRNA 902 | 902 | AACGGCACUGGGUACCGUG | 2953 | CACGGUACCCAGUGCCGUU |
| siRNA 908 | 908 | ACUGGGUACCGUGGCGUGG | 2959 | CCACGCCACGGUACCCAGU |
| siRNA 909 | 909 | CUGGGUACCGUGGCGUGGC | 2960 | GCCACGCCACGGUACCCAG |
| siRNA 910 | 910 | UGGGUACCGUGGCGUGGCC | 2961 | GGCCACGCCACGGUACCCA |
| siRNA 911 | 911 | GGGUACCGUGGCGUGGCCA | 2962 | UGGCCACGCCACGGUACCC |
| siRNA 916 | 916 | CCGUGGCGUGGCCAGCACC | 2967 | GGUGCUGGCCACGCCACGG |
| siRNA 957 | 957 | UGGCCUGGAACUCCGAUCU | 3008 | AGAUCGGAGUUCCAGGCCA |
| siRNA 960 | 960 | CCUGGAACUCCGAUCUGCU | 3011 | AGCAGAUCGGAGUUCCAGG |
| siRNA 961 | 961 | CUGGAACUCCGAUCUGCUC | 3012 | GAGCAGAUCGGAGUUCCAG |
| siRNA 964 | 964 | GAACUCCGAUCUGCUCUAC | 3015 | GUAGAGCAGAUCGGAGUUC |
| siRNA 992 | 992 | CACGUGGACUCCGUGGGCG | 3043 | CGCCCACGGAGUCCACGUG |
| siRNA 996 | 996 | UGGACUCCGUGGGCGCCGC | 3047 | GCGGCGCCCACGGAGUCCA |
| siRNA 998 | 998 | GACUCCGUGGGCGCCGCGG | 3049 | CCGCGGCGCCCACGGAGUC |
| siRNA 1033 | 1033 | CCCCCAUGCCUACUGCCGG | 3084 | CCGGCAGUAGGCAUGGGGG |
| siRNA 1034 | 1034 | CCCCAUGCCUACUGCCGGA | 3085 | UCCGGCAGUAGGCAUGGGG |
| siRNA 1035 | 1035 | CCCAUGCCUACUGCCGGAA | 3086 | UUCCGGCAGUAGGCAUGGG |
| siRNA 1040 | 1040 | GCCUACUGCCGGAAUCCGG | 3091 | CCGGAUUCCGGCAGUAGGC |
| siRNA 1041 | 1041 | CCUACUGCCGGAAUCCGGA | 3092 | UCCGGAUUCCGGCAGUAGG |
| siRNA 1042 | 1042 | CUACUGCCGGAAUCCGGAC | 3093 | GUCCGGAUUCCGGCAGUAG |
| siRNA 1043 | 1043 | UACUGCCGGAAUCCGGACA | 3094 | UGUCCGGAUUCCGGCAGUA |
| siRNA 1050 | 1050 | GGAAUCCGGACAAUGACGA | 3101 | UCGUCAUUGUCCGGAUUCC |
| siRNA 1051 | 1051 | GAAUCCGGACAAUGACGAG | 3102 | CUCGUCAUUGUCCGGAUUC |
| siRNA 1052 | 1052 | AAUCCGGACAAUGACGAGA | 3103 | UCUCGUCAUUGUCCGGAUU |
| siRNA 1053 | 1053 | AUCCGGACAAUGACGAGAG | 3104 | CUCUCGUCAUUGUCCGGAU |
| siRNA 1067 | 1067 | GAGAGGCCCUGGUGCUACG | 3118 | CGUAGCACCAGGGCCUCUC |
| siRNA 1100 | 1100 | GCGCUCUCCUGGGAGUACU | 3151 | AGUACUCCCAGGAGAGCGC |
| siRNA 1121 | 1121 | CGCCUGGAGGCCUGCGAAU | 3172 | AUUCGCAGGCCUCCAGGCG |
| siRNA 1122 | 1122 | GCCUGGAGGCCUGCGAAUC | 3173 | GAUUCGCAGGCCUCCAGGC |
| siRNA 1123 | 1123 | CCUGGAGGCCUGCGAAUCC | 3174 | GGAUUCGCAGGCCUCCAGG |
| siRNA 1133 | 1133 | UGCGAAUCCCUCACCAGAG | 3184 | CUCUGGUGAGGGAUUCGCA |
| siRNA 1143 | 1143 | UCACCAGAGUCCAACUGUC | 3194 | GACAGUUGGACUCUGGUGA |
| siRNA 1149 | 1149 | GAGUCCAACUGUCACCGGA | 3200 | UCCGGUGACAGUUGGACUC |
| siRNA 1151 | 1151 | GUCCAACUGUCACCGGAUC | 3202 | GAUCCGGUGACAGUUGGAC |
| siRNA 1153 | 1153 | CCAACUGUCACCGGAUCUC | 3204 | GAGAUCCGGUGACAGUUGG |
| siRNA 1203 | 1203 | GGCGCCAGGCCUGCGGCAG | 3254 | CUGCCGCAGGCCUGGCGCC |
| siRNA 1215 | 1215 | GCGGCAGGAGGCACAAGAA | 3266 | UUCUUGUGCCUCCUGCCGC |
| siRNA 1224 | 1224 | GGCACAAGAAGAGGACGUU | 3275 | AACGUCCUCUUCUUGUGCC |
| siRNA 1225 | 1225 | GCACAAGAAGAGGACGUUC | 3276 | GAACGUCCUCUUCUUGUGC |
| siRNA 1226 | 1226 | CACAAGAAGAGGACGUUCC | 3277 | GGAACGUCCUCUUCUUGUG |
| siRNA 1235 | 1235 | AGGACGUUCCUGCGGCCAC | 3286 | GUGGCCGCAGGAACGUCCU |
| siRNA 1238 | 1238 | ACGUUCCUGCGGCCACGUA | 3289 | UACGUGGCCGCAGGAACGU |
| siRNA 1240 | 1240 | GUUCCUGCGGCCACGUAUC | 3291 | GAUACGUGGCCGCAGGAAC |
| siRNA 1242 | 1242 | UCCUGCGGCCACGUAUCAU | 3293 | AUGAUACGUGGCCGCAGGA |
| siRNA 1243 | 1243 | CCUGCGGCCACGUAUCAUC | 3294 | GAUGAUACGUGGCCGCAGG |
| siRNA 1246 | 1246 | GCGGCCACGUAUCAUCGGC | 3297 | GCCGAUGAUACGUGGCCGC |
| siRNA 1247 | 1247 | CGGCCACGUAUCAUCGGCG | 3298 | CGCCGAUGAUACGUGGCCG |
| siRNA 1248 | 1248 | GGCCACGUAUCAUCGGCGG | 3299 | CCGCCGAUGAUACGUGGCC |
| siRNA 1249 | 1249 | GCCACGUAUCAUCGGCGGC | 3300 | GCCGCCGAUGAUACGUGGC |
| siRNA 1274 | 1274 | UCGCUGCCCGGCUCGCACC | 3325 | GGUGCGAGCCGGGCAGCGA |
| siRNA 1299 | 1299 | UGGCCGCCAUCUACAUCGG | 3350 | CCGAUGUAGAUGGCGGCCA |
| siRNA 1312 | 1312 | CAUCGGGGACAGCUUCUGC | 3363 | GCAGAAGCUGUCCCCGAUG |
| siRNA 1315 | 1315 | CGGGGACAGCUUCUGCGCC | 3366 | GGCGCAGAAGCUGUCCCCG |
| siRNA 1318 | 1318 | GGACAGCUUCUGCGCCGGG | 3369 | CCCGGCGCAGAAGCUGUCC |
| siRNA 1319 | 1319 | GACAGCUUCUGCGCCGGGA | 3370 | UCCCGGCGCAGAAGCUGUC |
| siRNA 1352 | 1352 | UGCUGGGUGGUGUCGGCCG | 3403 | CGGCCGACACCACCCAGCA |
| siRNA 1389 | 1389 | GCCCCCCCAGGGACAGCGU | 3440 | ACGCUGUCCCUGGGGGGGC |
| siRNA 1390 | 1390 | CCCCCCCAGGGACAGCGUC | 3441 | GACGCUGUCCCUGGGGGGG |
| siRNA 1392 | 1392 | CCCCCAGGGACAGCGUCUC | 3443 | GAGACGCUGUCCCUGGGGG |
| siRNA 1424 | 1424 | CAGCACUUCUUCAACCGCA | 3475 | UGCGGUUGAAGAAGUGCUG |
| siRNA 1427 | 1427 | CACUUCUUCAACCGCACGA | 3478 | UCGUGCGGUUGAAGAAGUG |
| siRNA 1430 | 1430 | UUCUUCAACCGCACGACGG | 3481 | CCGUCGUGCGGUUGAAGAA |
| siRNA 1431 | 1431 | UCUUCAACCGCACGACGGA | 3482 | UCCGUCGUGCGGUUGAAGA |
| siRNA 1432 | 1432 | CUUCAACCGCACGACGGAC | 3483 | GUCCGUCGUGCGGUUGAAG |
| siRNA 1433 | 1433 | UUCAACCGCACGACGGACG | 3484 | CGUCCGUCGUGCGGUUGAA |
| siRNA 1434 | 1434 | UCAACCGCACGACGGACGU | 3485 | ACGUCCGUCGUGCGGUUGA |
| siRNA 1435 | 1435 | CAACCGCACGACGGACGUG | 3486 | CACGUCCGUCGUGCGGUUG |
| siRNA 1438 | 1438 | CCGCACGACGGACGUGACG | 3489 | CGUCACGUCCGUCGUGCGG |
| siRNA 1439 | 1439 | CGCACGACGGACGUGACGC | 3490 | GCGUCACGUCCGUCGUGCG |
| siRNA 1440 | 1440 | GCACGACGGACGUGACGCA | 3491 | UGCGUCACGUCCGUCGUGC |
| siRNA 1441 | 1441 | CACGACGGACGUGACGCAG | 3492 | CUGCGUCACGUCCGUCGUG |
| siRNA 1443 | 1443 | CGACGGACGUGACGCAGAC | 3494 | GUCUGCGUCACGUCCGUCG |
| siRNA 1446 | 1446 | CGGACGUGACGCAGACCUU | 3497 | AAGGUCUGCGUCACGUCCG |
| siRNA 1449 | 1449 | ACGUGACGCAGACCUUCGG | 3500 | CCGAAGGUCUGCGUCACGU |
| siRNA 1452 | 1452 | UGACGCAGACCUUCGGCAU | 3503 | AUGCCGAAGGUCUGCGUCA |
| siRNA 1453 | 1453 | GACGCAGACCUUCGGCAUC | 3504 | GAUGCCGAAGGUCUGCGUC |
| siRNA 1455 | 1455 | CGCAGACCUUCGGCAUCGA | 3506 | UCGAUGCCGAAGGUCUGCG |
| siRNA 1456 | 1456 | GCAGACCUUCGGCAUCGAG | 3507 | CUCGAUGCCGAAGGUCUGC |
| siRNA 1459 | 1459 | GACCUUCGGCAUCGAGAAG | 3510 | CUUCUCGAUGCCGAAGGUC |
| siRNA 1463 | 1463 | UUCGGCAUCGAGAAGUACA | 3514 | UGUACUUCUCGAUGCCGAA |
| siRNA 1464 | 1464 | UCGGCAUCGAGAAGUACAU | 3515 | AUGUACUUCUCGAUGCCGA |
| siRNA 1469 | 1469 | AUCGAGAAGUACAUCCCGU | 3520 | ACGGGAUGUACUUCUCGAU |
| siRNA 1471 | 1471 | CGAGAAGUACAUCCCGUAC | 3522 | GUACGGGAUGUACUUCUCG |
| siRNA 1475 | 1475 | AAGUACAUCCCGUACACCC | 3526 | GGGUGUACGGGAUGUACUU |
| siRNA 1477 | 1477 | GUACAUCCCGUACACCCUG | 3528 | CAGGGUGUACGGGAUGUAC |
| siRNA 1504 | 1504 | GUUCAACCCCAGCGACCAC | 3555 | GUGGUCGCUGGGGUUGAAC |
| siRNA 1506 | 1506 | UCAACCCCAGCGACCACGA | 3557 | UCGUGGUCGCUGGGGUUGA |
| siRNA 1509 | 1509 | ACCCCAGCGACCACGACCU | 3560 | AGGUCGUGGUCGCUGGGGU |
| siRNA 1512 | 1512 | CCAGCGACCACGACCUCGU | 3563 | ACGAGGUCGUGGUCGCUGG |
| siRNA 1513 | 1513 | CAGCGACCACGACCUCGUC | 3564 | GACGAGGUCGUGGUCGCUG |
| siRNA 1516 | 1516 | CGACCACGACCUCGUCCUG | 3567 | CAGGACGAGGUCGUGGUCG |
| siRNA 1518 | 1518 | ACCACGACCUCGUCCUGAU | 3569 | AUCAGGACGAGGUCGUGGU |
| siRNA 1519 | 1519 | CCACGACCUCGUCCUGAUC | 3570 | GAUCAGGACGAGGUCGUGG |
| siRNA 1521 | 1521 | ACGACCUCGUCCUGAUCCG | 3572 | CGGAUCAGGACGAGGUCGU |
| siRNA 1523 | 1523 | GACCUCGUCCUGAUCCGGC | 3574 | GCCGGAUCAGGACGAGGUC |
| siRNA 1524 | 1524 | ACCUCGUCCUGAUCCGGCU | 3575 | AGCCGGAUCAGGACGAGGU |
| siRNA 1528 | 1528 | CGUCCUGAUCCGGCUGAAG | 3579 | CUUCAGCCGGAUCAGGACG |
| siRNA 1563 | 1563 | GUGCCACACGCUCGCAGUU | 3614 | AACUGCGAGCGUGUGGCAC |
| siRNA 1564 | 1564 | UGCCACACGCUCGCAGUUC | 3615 | GAACUGCGAGCGUGUGGCA |
| siRNA 1565 | 1565 | GCCACACGCUCGCAGUUCG | 3616 | CGAACUGCGAGCGUGUGGC |
| siRNA 1566 | 1566 | CCACACGCUCGCAGUUCGU | 3617 | ACGAACUGCGAGCGUGUGG |
| siRNA 1567 | 1567 | CACACGCUCGCAGUUCGUG | 3618 | CACGAACUGCGAGCGUGUG |
| siRNA 1568 | 1568 | ACACGCUCGCAGUUCGUGC | 3619 | GCACGAACUGCGAGCGUGU |
| siRNA 1572 | 1572 | GCUCGCAGUUCGUGCAGCC | 3623 | GGCUGCACGAACUGCGAGC |
| siRNA 1622 | 1622 | CCCGCAGGACACAAGUGCC | 3673 | GGCACUUGUGUCCUGCGGG |
| siRNA 1623 | 1623 | CCGCAGGACACAAGUGCCA | 3674 | UGGCACUUGUGUCCUGCGG |
| siRNA 1624 | 1624 | CGCAGGACACAAGUGCCAG | 3675 | CUGGCACUUGUGUCCUGCG |
| siRNA 1628 | 1628 | GGACACAAGUGCCAGAUUG | 3679 | CAAUCUGGCACUUGUGUCC |
| siRNA 1630 | 1630 | ACACAAGUGCCAGAUUGCG | 3681 | CGCAAUCUGGCACUUGUGU |
| siRNA 1631 | 1631 | CACAAGUGCCAGAUUGCGG | 3682 | CCGCAAUCUGGCACUUGUG |
| siRNA 1651 | 1651 | CUGGGGCCACUUGGAUGAG | 3702 | CUCAUCCAAGUGGCCCCAG |
| siRNA 1662 | 1662 | UGGAUGAGAACGUGAGCGG | 3713 | CCGCUCACGUUCUCAUCCA |
| siRNA 1663 | 1663 | GGAUGAGAACGUGAGCGGC | 3714 | GCCGCUCACGUUCUCAUCC |
| siRNA 1672 | 1672 | CGUGAGCGGCUACUCCAGC | 3723 | GCUGGAGUAGCCGCUCACG |
| siRNA 1709 | 1709 | GUCCCCCUGGUCGCCGACC | 3760 | GGUCGGCGACCAGGGGGAC |
| siRNA 1712 | 1712 | CCCCUGGUCGCCGACCACA | 3763 | UGUGGUCGGCGACCAGGGG |
| siRNA 1713 | 1713 | CCCUGGUCGCCGACCACAA | 3764 | UUGUGGUCGGCGACCAGGG |
| siRNA 1716 | 1716 | UGGUCGCCGACCACAAGUG | 3767 | CACUUGUGGUCGGCGACCA |
| siRNA 1718 | 1718 | GUCGCCGACCACAAGUGCA | 3769 | UGCACUUGUGGUCGGCGAC |
| siRNA 1722 | 1722 | CCGACCACAAGUGCAGCAG | 3773 | CUGCUGCACUUGUGGUCGG |
| siRNA 1737 | 1737 | GCAGCCCUGAGGUCUACGG | 3788 | CCGUAGACCUCAGGGCUGC |
| siRNA 1739 | 1739 | AGCCCUGAGGUCUACGGCG | 3790 | CGCCGUAGACCUCAGGGCU |
| siRNA 1743 | 1743 | CUGAGGUCUACGGCGCCGA | 3794 | UCGGCGCCGUAGACCUCAG |
| siRNA 1745 | 1745 | GAGGUCUACGGCGCCGACA | 3796 | UGUCGGCGCCGUAGACCUC |
| siRNA 1749 | 1749 | UCUACGGCGCCGACAUCAG | 3800 | CUGAUGUCGGCGCCGUAGA |
| siRNA 1752 | 1752 | ACGGCGCCGACAUCAGCCC | 3803 | GGGCUGAUGUCGGCGCCGU |
| siRNA 1770 | 1770 | CCAACAUGCUCUGUGCCGG | 3821 | CCGGCACAGAGCAUGUUGG |
| siRNA 1780 | 1780 | CUGUGCCGGCUACUUCGAC | 3831 | GUCGAAGUAGCCGGCACAG |
| siRNA 1781 | 1781 | UGUGCCGGCUACUUCGACU | 3832 | AGUCGAAGUAGCCGGCACA |
| siRNA 1782 | 1782 | GUGCCGGCUACUUCGACUG | 3833 | CAGUCGAAGUAGCCGGCAC |
| siRNA 1783 | 1783 | UGCCGGCUACUUCGACUGC | 3834 | GCAGUCGAAGUAGCCGGCA |
| siRNA 1785 | 1785 | CCGGCUACUUCGACUGCAA | 3836 | UUGCAGUCGAAGUAGCCGG |
| siRNA 1788 | 1788 | GCUACUUCGACUGCAAGUC | 3839 | GACUUGCAGUCGAAGUAGC |
| siRNA 1789 | 1789 | CUACUUCGACUGCAAGUCC | 3840 | GGACUUGCAGUCGAAGUAG |
| siRNA 1791 | 1791 | ACUUCGACUGCAAGUCCGA | 3842 | UCGGACUUGCAGUCGAAGU |
| siRNA 1792 | 1792 | CUUCGACUGCAAGUCCGAC | 3843 | GUCGGACUUGCAGUCGAAG |
| siRNA 1793 | 1793 | UUCGACUGCAAGUCCGACG | 3844 | CGUCGGACUUGCAGUCGAA |
| siRNA 1794 | 1794 | UCGACUGCAAGUCCGACGC | 3845 | GCGUCGGACUUGCAGUCGA |
| siRNA 1795 | 1795 | CGACUGCAAGUCCGACGCC | 3846 | GGCGUCGGACUUGCAGUCG |
| siRNA 1799 | 1799 | UGCAAGUCCGACGCCUGCC | 3850 | GGCAGGCGUCGGACUUGCA |
| siRNA 1840 | 1840 | GGCCUGCGAGAAGAACGGC | 3891 | GCCGUUCUUCUCGCAGGCC |
| siRNA 1843 | 1843 | CUGCGAGAAGAACGGCGUG | 3894 | CACGCCGUUCUUCUCGCAG |
| siRNA 1849 | 1849 | GAAGAACGGCGUGGCUUAC | 3900 | GUAAGCCACGCCGUUCUUC |
| siRNA 1850 | 1850 | AAGAACGGCGUGGCUUACC | 3901 | GGUAAGCCACGCCGUUCUU |
| siRNA 1851 | 1851 | AGAACGGCGUGGCUUACCU | 3902 | AGGUAAGCCACGCCGUUCU |
| siRNA 1857 | 1857 | GCGUGGCUUACCUCUACGG | 3908 | CCGUAGAGGUAAGCCACGC |
| siRNA 1858 | 1858 | CGUGGCUUACCUCUACGGC | 3909 | GCCGUAGAGGUAAGCCACG |
| siRNA 1859 | 1859 | GUGGCUUACCUCUACGGCA | 3910 | UGCCGUAGAGGUAAGCCAC |
| siRNA 1860 | 1860 | UGGCUUACCUCUACGGCAU | 3911 | AUGCCGUAGAGGUAAGCCA |
| siRNA 1861 | 1861 | GGCUUACCUCUACGGCAUC | 3912 | GAUGCCGUAGAGGUAAGCC |
| siRNA 1862 | 1862 | GCUUACCUCUACGGCAUCA | 3913 | UGAUGCCGUAGAGGUAAGC |
| siRNA 1863 | 1863 | CUUACCUCUACGGCAUCAU | 3914 | AUGAUGCCGUAGAGGUAAG |
| siRNA 1897 | 1897 | CUGCGGGCGGCUCCACAAG | 3948 | CUUGUGGAGCCGCCCGCAG |
| siRNA 1900 | 1900 | CGGGCGGCUCCACAAGCCG | 3951 | CGGCUUGUGGAGCCGCCCG |
| siRNA 1901 | 1901 | GGGCGGCUCCACAAGCCGG | 3952 | CCGGCUUGUGGAGCCGCCC |
| siRNA 1902 | 1902 | GGCGGCUCCACAAGCCGGG | 3953 | CCCGGCUUGUGGAGCCGCC |
| siRNA 1903 | 1903 | GCGGCUCCACAAGCCGGGG | 3954 | CCCCGGCUUGUGGAGCCGC |
| siRNA 1908 | 1908 | UCCACAAGCCGGGGGUCUA | 3959 | UAGACCCCCGGCUUGUGGA |
| siRNA 1910 | 1910 | CACAAGCCGGGGGUCUACA | 3961 | UGUAGACCCCCGGCUUGUG |
| siRNA 1917 | 1917 | CGGGGGUCUACACCCGCGU | 3968 | ACGCGGGUGUAGACCCCCG |
| siRNA 1927 | 1927 | CACCCGCGUGGCCAACUAU | 3978 | AUAGUUGGCCACGCGGGUG |
| siRNA 1928 | 1928 | ACCCGCGUGGCCAACUAUG | 3979 | CAUAGUUGGCCACGCGGGU |
| siRNA 1929 | 1929 | CCCGCGUGGCCAACUAUGU | 3980 | ACAUAGUUGGCCACGCGGG |
| siRNA 1937 | 1937 | GCCAACUAUGUGGACUGGA | 3988 | UCCAGUCCACAUAGUUGGC |
| siRNA 1974 | 1974 | CUCCCAGGCGGCUUGUGGC | 4025 | GCCACAAGCCGCCUGGGAG |
| siRNA 2010 | 2010 | GCGGGACACCCUGGUUCCC | 4061 | GGGAACCAGGGUGUCCCGC |

The siRNAs in subset C had the following characteristics: Cross-reactivity: With 19mer in human HGFAC mRNA; Specificity category: For human: AS2 or better, SS3 or better; miRNA seeds: AS+SS strand: seed region not conserved in human, mouse, and rat and not present in >4 species. AS strand: seed region not identical to seed region of known human miRNA; Off-target frequency: ≤30 human off-targets matched with 2 mismatches by antisense strand; and SNPs: siRNA target sites do not harbor SNPs with a MAF≥1% (pos. 2-18).

siRNA sequences in subset C were also selected for absence of seed regions in the AS or S strands that are identical to a seed region of known human miRNA in addition to having an off-target frequency of ≤20 human off-targets matched with 2 mismatches by antisense strand to yield subset D. Subset D includes 154 siRNAs whose base sequences are shown in Table D.

TABLE D
Subset D

| | SEQ ID | sense strand | SEQ ID | antisense strand |
|---|---|---|---|---|
| siRNA NO: | NO: | sequence (5′-3′) | NO: | sequence (5′-3′) |
| siRNA 116 | 116 | CUGCUGCCACGGGGGUUCC | 2167 | GGAACCCCCGUGGCAGCAG |
| siRNA 136 | 136 | GCCCCAGCCUGGCGGGAAC | 2187 | GUUCCCGCCAGGCUGGGGC |
| siRNA 143 | 143 | CCUGGCGGGAACCGUACGG | 2194 | CCGUACGGUUCCCGCCAGG |
| siRNA 149 | 149 | GGGAACCGUACGGAGUCCC | 2200 | GGGACUCCGUACGGUUCCC |
| siRNA 155 | 155 | CGUACGGAGUCCCCAGAAC | 2206 | GUUCUGGGGACUCCGUACG |
| siRNA 158 | 158 | ACGGAGUCCCCAGAACCUA | 2209 | UAGGUUCUGGGGACUCCGU |
| siRNA 172 | 172 | ACCUAAUGCCACAGCGACC | 2223 | GGUCGCUGUGGCAUUAGGU |
| siRNA 202 | 202 | CACUAUCCUGGUGACCUCU | 2253 | AGAGGUCACCAGGAUAGUG |
| siRNA 232 | 232 | GACCCCAGCAACAAGUGCU | 2283 | AGCACUUGUUGCUGGGGUC |
| siRNA 328 | 328 | CCAAGCACUCACCGAGGAC | 2379 | GUCCUCGGUGAGUGCUUGG |
| siRNA 335 | 335 | CUCACCGAGGACGGGAGGC | 2386 | GCCUCCCGUCCUCGGUGAG |
| siRNA 371 | 371 | CGCUACGGGGGCCGCAUGC | 2422 | GCAUGCGGCCCCCGUAGCG |
| siRNA 372 | 372 | GCUACGGGGGCCGCAUGCU | 2423 | AGCAUGCGGCCCCCGUAGC |
| siRNA 373 | 373 | CUACGGGGGCCGCAUGCUG | 2424 | CAGCAUGCGGCCCCCGUAG |
| siRNA 391 | 391 | GCAUGCCUGCACUUCGGAG | 2442 | CUCCGAAGUGCAGGCAUGC |
| siRNA 398 | 398 | UGCACUUCGGAGGGCAGUG | 2449 | CACUGCCCUCCGAAGUGCA |
| siRNA 436 | 436 | CACAACUCACAACUACGAC | 2487 | GUCGUAGUUGUGAGUUGUG |
| siRNA 549 | 549 | GAGGCUCCUGCUCCAAUAC | 2600 | GUAUUGGAGCAGGAGCCUC |
| siRNA 569 | 569 | CAGGACCCCCAGUCCUAUC | 2620 | GAUAGGACUGGGGGUCCUG |
| siRNA 612 | 612 | CCGGCAAGGACUGCGGCAC | 2663 | GUGCCGCAGUCCUUGCCGG |
| siRNA 733 | 733 | CCGGACCUGGUGCGAAGGC | 2784 | GCCUUCGCACCAGGUCCGG |
| siRNA 742 | 742 | GUGCGAAGGCACCCGACAU | 2793 | AUGUCGGGUGCCUUCGCAC |
| siRNA 743 | 743 | UGCGAAGGCACCCGACAUA | 2794 | UAUGUCGGGUGCCUUCGCA |
| siRNA 744 | 744 | GCGAAGGCACCCGACAUAC | 2795 | GUAUGUCGGGUGCCUUCGC |
| siRNA 749 | 749 | GGCACCCGACAUACAGCUU | 2800 | AAGCUGUAUGUCGGGUGCC |
| siRNA 751 | 751 | CACCCGACAUACAGCUUGU | 2802 | ACAAGCUGUAUGUCGGGUG |
| siRNA 753 | 753 | CCCGACAUACAGCUUGUCU | 2804 | AGACAAGCUGUAUGUCGGG |
| siRNA 754 | 754 | CCGACAUACAGCUUGUCUG | 2805 | CAGACAAGCUGUAUGUCGG |
| siRNA 756 | 756 | GACAUACAGCUUGUCUGAG | 2807 | CUCAGACAAGCUGUAUGUC |
| siRNA 796 | 796 | CACCUGCCACCUGAUCGUG | 2847 | CACGAUCAGGUGGCAGGUG |
| siRNA 819 | 819 | CCGGGACCACCGUGUGUGC | 2870 | GCACACACGGUGGUCCCGG |
| siRNA 857 | 857 | GGACGGCUCUGCAACAUCG | 2908 | CGAUGUUGCAGAGCCGUCC |
| siRNA 858 | 858 | GACGGCUCUGCAACAUCGA | 2909 | UCGAUGUUGCAGAGCCGUC |
| siRNA 863 | 863 | CUCUGCAACAUCGAGCCUG | 2914 | CAGGCUCGAUGUUGCAGAG |
| siRNA 871 | 871 | CAUCGAGCCUGAUGAGCGC | 2922 | GCGCUCAUCAGGCUCGAUG |
| siRNA 872 | 872 | AUCGAGCCUGAUGAGCGCU | 2923 | AGCGCUCAUCAGGCUCGAU |
| siRNA 873 | 873 | UCGAGCCUGAUGAGCGCUG | 2924 | CAGCGCUCAUCAGGCUCGA |
| siRNA 879 | 879 | CUGAUGAGCGCUGCUUCUU | 2930 | AAGAAGCAGCGCUCAUCAG |
| siRNA 910 | 910 | UGGGUACCGUGGCGUGGCC | 2961 | GGCCACGCCACGGUACCCA |
| siRNA 911 | 911 | GGGUACCGUGGCGUGGCCA | 2962 | UGGCCACGCCACGGUACCC |
| siRNA 961 | 961 | CUGGAACUCCGAUCUGCUC | 3012 | GAGCAGAUCGGAGUUCCAG |
| siRNA 964 | 964 | GAACUCCGAUCUGCUCUAC | 3015 | GUAGAGCAGAUCGGAGUUC |
| siRNA 992 | 992 | CACGUGGACUCCGUGGGCG | 3043 | CGCCCACGGAGUCCACGUG |
| siRNA 996 | 996 | UGGACUCCGUGGGCGCCGC | 3047 | GCGGCGCCCACGGAGUCCA |
| siRNA 998 | 998 | GACUCCGUGGGCGCCGCGG | 3049 | CCGCGGCGCCCACGGAGUC |
| siRNA 1033 | 1033 | CCCCCAUGCCUACUGCCGG | 3084 | CCGGCAGUAGGCAUGGGGG |
| siRNA 1034 | 1034 | CCCCAUGCCUACUGCCGGA | 3085 | UCCGGCAGUAGGCAUGGGG |
| siRNA 1040 | 1040 | GCCUACUGCCGGAAUCCGG | 3091 | CCGGAUUCCGGCAGUAGGC |
| siRNA 1041 | 1041 | CCUACUGCCGGAAUCCGGA | 3092 | UCCGGAUUCCGGCAGUAGG |
| siRNA 1042 | 1042 | CUACUGCCGGAAUCCGGAC | 3093 | GUCCGGAUUCCGGCAGUAG |
| siRNA 1043 | 1043 | UACUGCCGGAAUCCGGACA | 3094 | UGUCCGGAUUCCGGCAGUA |
| siRNA 1050 | 1050 | GGAAUCCGGACAAUGACGA | 3101 | UCGUCAUUGUCCGGAUUCC |
| siRNA 1051 | 1051 | GAAUCCGGACAAUGACGAG | 3102 | CUCGUCAUUGUCCGGAUUC |
| siRNA 1052 | 1052 | AAUCCGGACAAUGACGAGA | 3103 | UCUCGUCAUUGUCCGGAUU |
| siRNA 1053 | 1053 | AUCCGGACAAUGACGAGAG | 3104 | CUCUCGUCAUUGUCCGGAU |
| siRNA 1067 | 1067 | GAGAGGCCCUGGUGCUACG | 3118 | CGUAGCACCAGGGCCUCUC |
| siRNA 1123 | 1123 | CCUGGAGGCCUGCGAAUCC | 3174 | GGAUUCGCAGGCCUCCAGG |
| siRNA 1133 | 1133 | UGCGAAUCCCUCACCAGAG | 3184 | CUCUGGUGAGGGAUUCGCA |
| siRNA 1149 | 1149 | GAGUCCAACUGUCACCGGA | 3200 | UCCGGUGACAGUUGGACUC |
| siRNA 1151 | 1151 | GUCCAACUGUCACCGGAUC | 3202 | GAUCCGGUGACAGUUGGAC |
| siRNA 1153 | 1153 | CCAACUGUCACCGGAUCUC | 3204 | GAGAUCCGGUGACAGUUGG |
| siRNA 1203 | 1203 | GGCGCCAGGCCUGCGGCAG | 3254 | CUGCCGCAGGCCUGGCGCC |
| siRNA 1215 | 1215 | GCGGCAGGAGGCACAAGAA | 3266 | UUCUUGUGCCUCCUGCCGC |
| siRNA 1224 | 1224 | GGCACAAGAAGAGGACGUU | 3275 | AACGUCCUCUUCUUGUGCC |
| siRNA 1226 | 1226 | CACAAGAAGAGGACGUUCC | 3277 | GGAACGUCCUCUUCUUGUG |
| siRNA 1235 | 1235 | AGGACGUUCCUGCGGCCAC | 3286 | GUGGCCGCAGGAACGUCCU |
| siRNA 1238 | 1238 | ACGUUCCUGCGGCCACGUA | 3289 | UACGUGGCCGCAGGAACGU |
| siRNA 1246 | 1246 | GCGGCCACGUAUCAUCGGC | 3297 | GCCGAUGAUACGUGGCCGC |
| siRNA 1247 | 1247 | CGGCCACGUAUCAUCGGCG | 3298 | CGCCGAUGAUACGUGGCCG |
| siRNA 1248 | 1248 | GGCCACGUAUCAUCGGCGG | 3299 | CCGCCGAUGAUACGUGGCC |
| siRNA 1249 | 1249 | GCCACGUAUCAUCGGCGGC | 3300 | GCCGCCGAUGAUACGUGGC |
| siRNA 1274 | 1274 | UCGCUGCCCGGCUCGCACC | 3325 | GGUGCGAGCCGGGCAGCGA |
| siRNA 1299 | 1299 | UGGCCGCCAUCUACAUCGG | 3350 | CCGAUGUAGAUGGCGGCCA |
| siRNA 1312 | 1312 | CAUCGGGGACAGCUUCUGC | 3363 | GCAGAAGCUGUCCCCGAUG |
| siRNA 1319 | 1319 | GACAGCUUCUGCGCCGGGA | 3370 | UCCCGGCGCAGAAGCUGUC |
| siRNA 1389 | 1389 | GCCCCCCCAGGGACAGCGU | 3440 | ACGCUGUCCCUGGGGGGGC |
| siRNA 1390 | 1390 | CCCCCCCAGGGACAGCGUC | 3441 | GACGCUGUCCCUGGGGGGG |
| siRNA 1392 | 1392 | CCCCCAGGGACAGCGUCUC | 3443 | GAGACGCUGUCCCUGGGGG |
| siRNA 1424 | 1424 | CAGCACUUCUUCAACCGCA | 3475 | UGCGGUUGAAGAAGUGCUG |
| siRNA 1427 | 1427 | CACUUCUUCAACCGCACGA | 3478 | UCGUGCGGUUGAAGAAGUG |
| siRNA 1430 | 1430 | UUCUUCAACCGCACGACGG | 3481 | CCGUCGUGCGGUUGAAGAA |
| siRNA 1432 | 1432 | CUUCAACCGCACGACGGAC | 3483 | GUCCGUCGUGCGGUUGAAG |
| siRNA 1433 | 1433 | UUCAACCGCACGACGGACG | 3484 | CGUCCGUCGUGCGGUUGAA |
| siRNA 1434 | 1434 | UCAACCGCACGACGGACGU | 3485 | ACGUCCGUCGUGCGGUUGA |
| siRNA 1435 | 1435 | CAACCGCACGACGGACGUG | 3486 | CACGUCCGUCGUGCGGUUG |
| siRNA 1438 | 1438 | CCGCACGACGGACGUGACG | 3489 | CGUCACGUCCGUCGUGCGG |
| siRNA 1439 | 1439 | CGCACGACGGACGUGACGC | 3490 | GCGUCACGUCCGUCGUGCG |
| siRNA 1440 | 1440 | GCACGACGGACGUGACGCA | 3491 | UGCGUCACGUCCGUCGUGC |
| siRNA 1441 | 1441 | CACGACGGACGUGACGCAG | 3492 | CUGCGUCACGUCCGUCGUG |
| siRNA 1443 | 1443 | CGACGGACGUGACGCAGAC | 3494 | GUCUGCGUCACGUCCGUCG |
| siRNA 1446 | 1446 | CGGACGUGACGCAGACCUU | 3497 | AAGGUCUGCGUCACGUCCG |
| siRNA 1449 | 1449 | ACGUGACGCAGACCUUCGG | 3500 | CCGAAGGUCUGCGUCACGU |
| siRNA 1452 | 1452 | UGACGCAGACCUUCGGCAU | 3503 | AUGCCGAAGGUCUGCGUCA |
| siRNA 1453 | 1453 | GACGCAGACCUUCGGCAUC | 3504 | GAUGCCGAAGGUCUGCGUC |
| siRNA 1456 | 1456 | GCAGACCUUCGGCAUCGAG | 3507 | CUCGAUGCCGAAGGUCUGC |
| siRNA 1459 | 1459 | GACCUUCGGCAUCGAGAAG | 3510 | CUUCUCGAUGCCGAAGGUC |
| siRNA 1463 | 1463 | UUCGGCAUCGAGAAGUACA | 3514 | UGUACUUCUCGAUGCCGAA |
| siRNA 1469 | 1469 | AUCGAGAAGUACAUCCCGU | 3520 | ACGGGAUGUACUUCUCGAU |
| siRNA 1475 | 1475 | AAGUACAUCCCGUACACCC | 3526 | GGGUGUACGGGAUGUACUU |
| siRNA 1477 | 1477 | GUACAUCCCGUACACCCUG | 3528 | CAGGGUGUACGGGAUGUAC |
| siRNA 1504 | 1504 | GUUCAACCCCAGCGACCAC | 3555 | GUGGUCGCUGGGGUUGAAC |
| siRNA 1506 | 1506 | UCAACCCCAGCGACCACGA | 3557 | UCGUGGUCGCUGGGGUUGA |
| siRNA 1509 | 1509 | ACCCCAGCGACCACGACCU | 3560 | AGGUCGUGGUCGCUGGGGU |
| siRNA 1512 | 1512 | CCAGCGACCACGACCUCGU | 3563 | ACGAGGUCGUGGUCGCUGG |
| siRNA 1516 | 1516 | CGACCACGACCUCGUCCUG | 3567 | CAGGACGAGGUCGUGGUCG |
| siRNA 1518 | 1518 | ACCACGACCUCGUCCUGAU | 3569 | AUCAGGACGAGGUCGUGGU |
| siRNA 1519 | 1519 | CCACGACCUCGUCCUGAUC | 3570 | GAUCAGGACGAGGUCGUGG |
| siRNA 1521 | 1521 | ACGACCUCGUCCUGAUCCG | 3572 | CGGAUCAGGACGAGGUCGU |
| siRNA 1523 | 1523 | GACCUCGUCCUGAUCCGGC | 3574 | GCCGGAUCAGGACGAGGUC |
| siRNA 1524 | 1524 | ACCUCGUCCUGAUCCGGCU | 3575 | AGCCGGAUCAGGACGAGGU |
| siRNA 1528 | 1528 | CGUCCUGAUCCGGCUGAAG | 3579 | CUUCAGCCGGAUCAGGACG |
| siRNA 1564 | 1564 | UGCCACACGCUCGCAGUUC | 3615 | GAACUGCGAGCGUGUGGCA |
| siRNA 1565 | 1565 | GCCACACGCUCGCAGUUCG | 3616 | CGAACUGCGAGCGUGUGGC |
| siRNA 1566 | 1566 | CCACACGCUCGCAGUUCGU | 3617 | ACGAACUGCGAGCGUGUGG |
| siRNA 1567 | 1567 | CACACGCUCGCAGUUCGUG | 3618 | CACGAACUGCGAGCGUGUG |
| siRNA 1568 | 1568 | ACACGCUCGCAGUUCGUGC | 3619 | GCACGAACUGCGAGCGUGU |
| siRNA 1623 | 1623 | CCGCAGGACACAAGUGCCA | 3674 | UGGCACUUGUGUCCUGCGG |
| siRNA 1663 | 1663 | GGAUGAGAACGUGAGCGGC | 3714 | GCCGCUCACGUUCUCAUCC |
| siRNA 1672 | 1672 | CGUGAGCGGCUACUCCAGC | 3723 | GCUGGAGUAGCCGCUCACG |
| siRNA 1709 | 1709 | GUCCCCCUGGUCGCCGACC | 3760 | GGUCGGCGACCAGGGGGAC |
| siRNA 1713 | 1713 | CCCUGGUCGCCGACCACAA | 3764 | UUGUGGUCGGCGACCAGGG |
| siRNA 1718 | 1718 | GUCGCCGACCACAAGUGCA | 3769 | UGCACUUGUGGUCGGCGAC |
| siRNA 1722 | 1722 | CCGACCACAAGUGCAGCAG | 3773 | CUGCUGCACUUGUGGUCGG |
| sIRNA 1743 | 1743 | CUGAGGUCUACGGCGCCGA | 3794 | UCGGCGCCGUAGACCUCAG |
| siRNA 1745 | 1745 | GAGGUCUACGGCGCCGACA | 3796 | UGUCGGCGCCGUAGACCUC |
| siRNA 1749 | 1749 | UCUACGGCGCCGACAUCAG | 3800 | CUGAUGUCGGCGCCGUAGA |
| siRNA 1752 | 1752 | ACGGCGCCGACAUCAGCCC | 3803 | GGGCUGAUGUCGGCGCCGU |
| siRNA 1770 | 1770 | CCAACAUGCUCUGUGCCGG | 3821 | CCGGCACAGAGCAUGUUGG |
| siRNA 1781 | 1781 | UGUGCCGGCUACUUCGACU | 3832 | AGUCGAAGUAGCCGGCACA |
| siRNA 1782 | 1782 | GUGCCGGCUACUUCGACUG | 3833 | CAGUCGAAGUAGCCGGCAC |
| siRNA 1783 | 1783 | UGCCGGCUACUUCGACUGC | 3834 | GCAGUCGAAGUAGCCGGCA |
| siRNA 1785 | 1785 | CCGGCUACUUCGACUGCAA | 3836 | UUGCAGUCGAAGUAGCCGG |
| siRNA 1791 | 1791 | ACUUCGACUGCAAGUCCGA | 3842 | UCGGACUUGCAGUCGAAGU |
| siRNA 1792 | 1792 | CUUCGACUGCAAGUCCGAC | 3843 | GUCGGACUUGCAGUCGAAG |
| siRNA 1793 | 1793 | UUCGACUGCAAGUCCGACG | 3844 | CGUCGGACUUGCAGUCGAA |
| siRNA 1794 | 1794 | UCGACUGCAAGUCCGACGC | 3845 | GCGUCGGACUUGCAGUCGA |
| siRNA 1799 | 1799 | UGCAAGUCCGACGCCUGCC | 3850 | GGCAGGCGUCGGACUUGCA |
| siRNA 1850 | 1850 | AAGAACGGCGUGGCUUACC | 3901 | GGUAAGCCACGCCGUUCUU |
| siRNA 1851 | 1851 | AGAACGGCGUGGCUUACCU | 3902 | AGGUAAGCCACGCCGUUCU |
| siRNA 1858 | 1858 | CGUGGCUUACCUCUACGGC | 3909 | GCCGUAGAGGUAAGCCACG |
| siRNA 1859 | 1859 | GUGGCUUACCUCUACGGCA | 3910 | UGCCGUAGAGGUAAGCCAC |
| siRNA 1860 | 1860 | UGGCUUACCUCUACGGCAU | 3911 | AUGCCGUAGAGGUAAGCCA |
| siRNA 1861 | 1861 | GGCUUACCUCUACGGCAUC | 3912 | GAUGCCGUAGAGGUAAGCC |
| siRNA 1862 | 1862 | GCUUACCUCUACGGCAUCA | 3913 | UGAUGCCGUAGAGGUAAGC |
| siRNA 1863 | 1863 | CUUACCUCUACGGCAUCAU | 3914 | AUGAUGCCGUAGAGGUAAG |
| siRNA 1897 | 1897 | CUGCGGGCGGCUCCACAAG | 3948 | CUUGUGGAGCCGCCCGCAG |
| siRNA 1900 | 1900 | CGGGCGGCUCCACAAGCCG | 3951 | CGGCUUGUGGAGCCGCCCG |
| siRNA 1902 | 1902 | GGCGGCUCCACAAGCCGGG | 3953 | CCCGGCUUGUGGAGCCGCC |
| siRNA 1908 | 1908 | UCCACAAGCCGGGGGUCUA | 3959 | UAGACCCCCGGCUUGUGGA |
| siRNA 1910 | 1910 | CACAAGCCGGGGGUCUACA | 3961 | UGUAGACCCCCGGCUUGUG |
| siRNA 1927 | 1927 | CACCCGCGUGGCCAACUAU | 3978 | AUAGUUGGCCACGCGGGUG |
| siRNA 1929 | 1929 | CCCGCGUGGCCAACUAUGU | 3980 | ACAUAGUUGGCCACGCGGG |
| siRNA 1937 | 1937 | GCCAACUAUGUGGACUGGA | 3988 | UCCAGUCCACAUAGUUGGC |
| siRNA 2010 | 2010 | GCGGGACACCCUGGUUCCC | 4061 | GGGAACCAGGGUGUCCCGC |

Therapeutic siRNAs were designed to target human HGFAC as described above and, in some cases, the HGFAC sequence of at least one toxicology-relevant species, in this case, the non-human primate (NHP) cynomolgus monkey. The siRNAs included in subset E had the following characteristics: Cross-reactivity: With 19mer in human HGFAC mRNA, with 17mer/19mer in NHP HGFAC; Specificity category: For human and NHP: AS2 or better, SS3 or better; miRNA seeds: AS+SS strand: seed region not conserved in human, mouse, and rat and not present in >4 species; Off-target frequency: ≤20 human off-targets matched with 2 mismatches in antisense strand; and SNPs: siRNA target sites do not harbor SNPs with a MAF≥1% (pos. 2-18).

Subset E includes 26 siRNAs.

TABLE E
Subset E, Screening Set

| | | sense | | antisense |
|---|---|---|---|---|
| | SEQ | strand | SEQ | strand |
| | ID | sequence | ID | sequence |
| siRNA NO: | NO: | (5′-3′) | NO: | (5′-3′) |
| siRNA 160 | 160 | GGAGUCCCCAGA | 2211 | AUUAGGUUCUGG |
| | | ACCUAAU | | GGACUCC |
| siRNA 166 | 166 | CCCAGAACCUAA | 2217 | UGUGGCAUUAGG |
| | | UGCCACA | | UUCUGGG |
| siRNA 202 | 202 | CACUAUCCUGGU | 2253 | AGAGGUCACCAG |
| | | GACCUCU | | GAUAGUG |
| siRNA 203 | 203 | ACUAUCCUGGUG | 2254 | CAGAGGUCACCA |
| | | ACCUCUG | | GGAUAGU |
| siRNA 431 | 431 | UGUGCCACAACU | 2482 | AGUUGUGAGUUG |
| | | CACAACU | | UGGCACA |
| siRNA 434 | 434 | GCCACAACUCAC | 2485 | CGUAGUUGUGAG |
| | | AACUACG | | UUGUGGC |
| siRNA 436 | 436 | CACAACUCACAA | 2487 | GUCGUAGUUGUG |
| | | CUACGAC | | AGUUGUG |
| siRNA 437 | 437 | ACAACUCACAAC | 2488 | GGUCGUAGUUGU |
| | | UACGACC | | GAGUUGU |
| siRNA 438 | 438 | CAACUCACAACU | 2489 | CGGUCGUAGUUG |
| | | ACGACCG | | UGAGUUG |
| siRNA 751 | 751 | CACCCGACAUAC | 2802 | ACAAGCUGUAUG |
| | | AGCUUGU | | UCGGGUG |
| siRNA 879 | 879 | CUGAUGAGCGCU | 2930 | AAGAAGCAGCGC |
| | | GCUUCUU | | UCAUCAG |
| siRNA | 1424 | CAGCACUUCUUC | 3475 | UGCGGUUGAAGA |
| 1424 | | AACCGCA | | AGUGCUG |
| siRNA | 1428 | ACUUCUUCAACC | 3479 | GUCGUGCGGUUG |
| 1428 | | GCACGAC | | AAGAAGU |
| siRNA | 1460 | ACCUUCGGCAUC | 3511 | ACUUCUCGAUGC |
| 1460 | | GAGAAGU | | CGAAGGU |
| siRNA | 1463 | UUCGGCAUCGAG | 3514 | UGUACUUCUCGA |
| 1463 | | AAGUACA | | UGCCGAA |
| siRNA | 1464 | UCGGCAUCGAGA | 3515 | AUGUACUUCUCG |
| 1464 | | AGUACAU | | AUGCCGA |
| siRNA | 1469 | AUCGAGAAGUAC | 3520 | ACGGGAUGUACU |
| 1469 | | AUCCCGU | | UCUCGAU |
| siRNA | 1470 | UCGAGAAGUACA | 3521 | UACGGGAUGUAC |
| 1470 | | UCCCGUA | | UUCUCGA |
| siRNA | 1471 | CGAGAAGUACAU | 3522 | GUACGGGAUGUA |
| 1471 | | CCCGUAC | | CUUCUCG |
| siRNA | 1474 | GAAGUACAUCCC | 3525 | GGUGUACGGGAU |
| 1474 | | GUACACC | | GUACUUC |
| siRNA | 1628 | GGACACAAGUGC | 3679 | CAAUCUGGCACU |
| 1628 | | CAGAUUG | | UGUGUCC |
| siRNA | 1630 | ACACAAGUGCCA | 3681 | CGCAAUCUGGCA |
| 1630 | | GAUUGCG | | CUUGUGU |
| siRNA | 1788 | GCUACUUCGACU | 3839 | GACUUGCAGUCG |
| 1788 | | GCAAGUC | | AAGUAGC |
| siRNA | 1789 | CUACUUCGACUG | 3840 | GGACUUGCAGUC |
| 1789 | | CAAGUCC | | GAAGUAG |
| siRNA | 1791 | ACUUCGACUGCA | 3842 | UCGGACUUGCAG |
| 1791 | | AGUCCGA | | UCGAAGU |
| siRNA | 1864 | UUACCUCUACGG | 3915 | GAUGAUGCCGUA |
| 1864 | | CAUCAUC | | GAGGUAA |

In some cases, the sense strand of any of the siRNAs of subset E comprises siRNA with a particular modification pattern. In this modification pattern, position 9 counting from the 5′ end of the of the sense strand is has the 2′F modification. If position 9 of the sense strand is a pyrimidine, then all purines in the sense strand have the 2′OMe modification. If position 9 is the only pyrimidine between positions 5 and 11 of the sense stand, then position 9 is the only position with the 2′F modification in the sense strand. If position 9 and only one other base between positions 5 and 11 of the sense strand are pyrimidines, then both of these pyrimidines are the only two positions with the 2′F modification in the sense strand. If position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. If there are >2 pyrimidines between positions 5 and 11 of the sense strand, then all combinations of pyrimidines having the 2′F modification are allowed that have three to five 2′F modifications in total, provided that the sense strand does not have three 2′F modifications in a row. If position 9 of the sense strand is a purine, then all purines in the sense strand have the 2′OMe modification. If position 9 is the only purine between positions 5 and 11 of the sense stand, then position 9 is the only position with the 2′F modification in the sense strand. If position 9 and only one other base between positions 5 and 11 of the sense strand are purines, then both of these purines are the only two positions with the 2′F modification in the sense strand. If position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. If there are >2 purines between positions 5 and 11 of the sense strand, then all combinations of purines having the 2′F modification are allowed that have three to five 2′F modifications in total, provided that the sense strand does not have three 2′F modifications in a row.

In some cases, the sense strand of any of the siRNAs of subset E comprises a modification pattern which conforms to these sense strand rules (see, e.g., Table F).

In some cases, the antisense strand of any of the siRNAs of subset E comprise a modification or modification pattern. Some such examples are included in Table F. Table F(1) includes some additional details of the siRNAs in Table F. The modification pattern may include modification pattern 3AS. The siRNAs in subset E may comprise any other modification pattern(s).

TABLE F
Subset F, Mod Screening Set

| | SEQ ID | | SEQ ID | |
|---|---|---|---|---|
| siRNA name | NO: | sense strand sequence (5′-3′) | NO: | antisense strand sequence (5′-3′) |
| siRNA4103/4208 | 4103 | gsgsaguCfcCfCfagaaccuaaasusu | 4208 | usUfsuAfgGfuUfcUfgGfgGfaCfuCfcsusu |
| siRNA4104/4208 | 4104 | gsgsaguCfCfcCfagaaccuaaasusu | 4208 | usUfsuAfgGfuUfcUfgGfgGfaCfuCfcsusu |
| siRNA4105/4208 | 4105 | gsgsaguCfCfCfCfagaaccuaaasusu | 4208 | usUfsuAfgGfuUfcUfgGfgGfaCfuCfcsusu |
| siRNA4106/4208 | 4106 | gsgsagUfccCfCfagaaccuaaasusu | 4208 | usUfsuAfgGfuUfcUfgGfgGfaCfuCfcsusu |
| siRNA4107/4208 | 4107 | gsgsagUfcCfcCfagaaccuaaasusu | 4208 | usUfsuAfgGfuUfcUfgGfgGfaCfuCfcsusu |
| siRNA4108/4208 | 4108 | gsgsagUfCfccCfagaaccuaaasusu | 4208 | usUfsuAfgGfuUfcUfgGfgGfaCfuCfcsusu |
| siRNA4109/4208 | 4109 | gsgsagUfCfcCfCfagaaccuaaasusu | 4208 | usUfsuAfgGfuUfcUfgGfgGfaCfuCfcsusu |
| siRNA4110/4208 | 4110 | gsgsagUfCfCfCfCfagaaccuaaasusu | 4208 | usUfsuAfgGfuUfcUfgGfgGfaCfuCfcsusu |
| siRNA4111/4209 | 4111 | cscscagaacCfUfaaugccacasusu | 4209 | usGfsuGfgCfaUfuAfgGfuUfcUfgGfgsusu |
| siRNA4112/4209 | 4112 | cscscagaaCfCfuaaugccacasusu | 4209 | usGfsuGfgCfaUfuAfgGfuUfcUfgGfgsusu |
| siRNA4113/4210 | 4113 | csascuaUfcCfUfggugaccucasusu | 4210 | usGfsaGfgUfcAfcCfaGfgAfuAfgUfgsusu |
| siRNA4114/4210 | 4114 | csascuaUfCfcUfggugaccucasusu | 4210 | usGfsaGfgUfcAfcCfaGfgAfuAfgUfgsusu |
| siRNA4115/4210 | 4115 | csascuaUfCfCfUfggugaccucasusu | 4210 | usGfsaGfgUfcAfcCfaGfgAfuAfgUfgsusu |
| siRNA4116/4211 | 4116 | ascsuauccuGfGfugaccucuasusu | 4211 | usAfsgAfgGfuCfaCfcAfgGfaUfaGfususu |
| siRNA4117/4212 | 4117 | usgsugccAfcAfAfcucacaacasusu | 4212 | usGfsuUfgUfgAfgUfuGfuGfgCfaCfasusu |
| siRNA4118/4213 | 4118 | gscscaCfaacUfCfacaacuacasusu | 4213 | usGfsuAfgUfuGfuGfaGfuUfgUfgGfcsusu |
| siRNA4119/4213 | 4119 | gscscaCfaaCfUfcacaacuacasusu | 4213 | usGfsuAfgUfuGfuGfaGfuUfgUfgGfcsusu |
| siRNA4120/4214 | 4120 | csascaAfcucAfcAfacuacgaasusu | 4214 | usUfscGfuAfgUfuGfuGfaGfuUfgUfgsusu |
| siRNA4121/4215 | 4121 | ascsaacUfCfaCfaacuacgacasusu | 4215 | usGfsuCfgUfaGfuUfgUfgAfgUfuGfususu |
| siRNA4122/4215 | 4122 | ascsaaCfuCfaCfaacuacgacasusu | 4215 | usGfsuCfgUfaGfuUfgUfgAfgUfuGfususu |
| siRNA4123/4215 | 4123 | ascsaaCfUfcaCfaacuacgacasusu | 4215 | usGfsuCfgUfaGfuUfgUfgAfgUfuGfususu |
| siRNA4124/4216 | 4124 | csasacucAfcAfAfcuacgaccasusu | 4216 | usGfsgUfcGfuAfgUfuGfuGfaGfuUfgsusu |
| siRNA4125/4217 | 4125 | csascccgAfcAfuAfcagcuugasusu | 4217 | usCfsaAfgCfuGfuAfuGfuCfgGfgUfgsusu |
| siRNA4126/4217 | 4126 | csascccGfacAfuAfcagcuugasusu | 4217 | usCfsaAfgCfuGfuAfuGfuCfgGfgUfgsusu |
| siRNA4127/4217 | 4127 | csascccGfAfcAfuacagcuugasusu | 4217 | usCfsaAfgCfuGfuAfuGfuCfgGfgUfgsusu |
| siRNA4128/4217 | 4128 | csascccGfAfcAfuAfcagcuugasusu | 4217 | usCfsaAfgCfuGfuAfuGfuCfgGfgUfgsusu |
| siRNA4129/4218 | 4129 | csusgaUfgagCfgCfugcuucuasusu | 4218 | usAfsgAfaGfcAfgCfgCfuCfaUfcAfgsusu |
| siRNA4130/4219 | 4130 | csasgcacuUfCfuUfcaaccgcasusu | 4219 | usGfscGfgUfuGfaAfgAfaGfuGfcUfgsusu |
| siRNA4131/4219 | 4131 | csasgcacuUfCfUfUfcaaccgcasusu | 4219 | usGfscGfgUfuGfaAfgAfaGfuGfcUfgsusu |
| siRNA4132/4219 | 4132 | csasgcacUfuCfuUfcaaccgcasusu | 4219 | usGfscGfgUfuGfaAfgAfaGfuGfcUfgsusu |
| siRNA4133/4219 | 4133 | csasgcacUfuCfUfucaaccgcasusu | 4219 | usGfscGfgUfuGfaAfgAfaGfuGfcUfgsusu |
| siRNA4134/4219 | 4134 | csasgcacUfUfCfUfucaaccgcasusu | 4219 | usGfscGfgUfuGfaAfgAfaGfuGfcUfgsusu |
| siRNA4135/4219 | 4135 | csasgcacUfUfCfUfUfcaaccgcasusu | 4219 | usGfscGfgUfuGfaAfgAfaGfuGfcUfgsusu |
| siRNA4136/4219 | 4136 | csasgcaCfuuCfuUfcaaccgcasusu | 4219 | usGfscGfgUfuGfaAfgAfaGfuGfcUfgsusu |
| siRNA4137/4219 | 4137 | csasgcaCfuuCfUfucaaccgcasusu | 4219 | usGfscGfgUfuGfaAfgAfaGfuGfcUfgsusu |
| siRNA4138/4219 | 4138 | csasgcaCfuUfCfuucaaccgcasusu | 4219 | usGfscGfgUfuGfaAfgAfaGfuGfcUfgsusu |
| siRNA4139/4219 | 4139 | csasgcaCfuUfCfuUfcaaccgcasusu | 4219 | usGfscGfgUfuGfaAfgAfaGfuGfcUfgsusu |
| siRNA4140/4219 | 4140 | csasgcaCfuUfCfUfUfcaaccgcasusu | 4219 | usGfscGfgUfuGfaAfgAfaGfuGfcUfgsusu |
| siRNA4141/4219 | 4141 | csasgcaCfUfuCfuucaaccgcasusu | 4219 | usGfscGfgUfuGfaAfgAfaGfuGfcUfgsusu |
| siRNA4142/4219 | 4142 | csasgcaCfUfuCfuUfcaaccgcasusu | 4219 | usGfscGfgUfuGfaAfgAfaGfuGfcUfgsusu |
| siRNA4143/4219 | 4143 | csasgcaCfUfuCfUfucaaccgcasusu | 4219 | usGfscGfgUfuGfaAfgAfaGfuGfcUfgsusu |
| siRNA4144/4219 | 4144 | csasgcaCfUfUfCfuucaaccgcasusu | 4219 | usGfscGfgUfuGfaAfgAfaGfuGfcUfgsusu |
| siRNA4145/4219 | 4145 | csasgcaCfUfUfCfuUfcaaccgcasusu | 4219 | usGfscGfgUfuGfaAfgAfaGfuGfcUfgsusu |
| siRNA4146/4219 | 4146 | csasgcaCfUfUfCfUfucaaccgcasusu | 4219 | usGfscGfgUfuGfaAfgAfaGfuGfcUfgsusu |
| siRNA4147/4220 | 4147 | ascsuucuucAfAfccgcacgaasusu | 4220 | usUfscGfuGfcGfgUfuGfaAfgAfaGfususu |
| siRNA4148/4221 | 4148 | ascscuuCfggCfaUfcgagaagasusu | 4221 | usCfsuUfcUfcGfaUfgCfcGfaAfgGfususu |
| siRNA4149/4221 | 4149 | ascscuUfcggCfaUfcgagaagasusu | 4221 | usCfsuUfcUfcGfaUfgCfcGfaAfgGfususu |
| siRNA4150/4221 | 4150 | ascscuUfCfggCfaucgagaagasusu | 4221 | usCfsuUfcUfcGfaUfgCfcGfaAfgGfususu |
| siRNA4151/4221 | 4151 | ascscuUfCfggCfaUfcgagaagasusu | 4221 | usCfsuUfcUfcGfaUfgCfcGfaAfgGfususu |
| siRNA4152/4222 | 4152 | ususcggCfaUfCfgagaaguacasusu | 4222 | usGfsuAfcUfuCfuCfgAfuGfcCfgAfasusu |
| siRNA4153/4223 | 4153 | uscsggcAfucGfaGfaaguacaasusu | 4223 | usUfsgUfaCfuUfcUfcGfaUfgCfcGfasusu |
| siRNA4154/4223 | 4154 | uscsggcAfucGfAfgaaguacaasusu | 4223 | usUfsgUfaCfuUfcUfcGfaUfgCfcGfasusu |
| siRNA4155/4224 | 4155 | asuscgagaAfGfuAfcaucccgasusu | 4224 | usCfsgGfgAfuGfuAfcUfuCfuCfgAfususu |
| siRNA4156/4224 | 4156 | asuscgagAfaGfuAfcaucccgasusu | 4224 | usCfsgGfgAfuGfuAfcUfuCfuCfgAfususu |
| siRNA4157/4224 | 4157 | asuscgaGfaaGfuAfcaucccgasusu | 4224 | usCfsgGfgAfuGfuAfcUfuCfuCfgAfususu |
| siRNA4158/4224 | 4158 | asuscgaGfaAfGfuacaucccgasusu | 4224 | usCfsgGfgAfuGfuAfcUfuCfuCfgAfususu |
| siRNA4159/4224 | 4159 | asuscgaGfaAfGfuAfcaucccgasusu | 4224 | usCfsgGfgAfuGfuAfcUfuCfuCfgAfususu |
| siRNA4160/4224 | 4160 | asuscgaGfAfaGfuacaucccgasusu | 4224 | usCfsgGfgAfuGfuAfcUfuCfuCfgAfususu |
| siRNA4161/4224 | 4161 | asuscgaGfAfaGfuAfcaucccgasusu | 4224 | usCfsgGfgAfuGfuAfcUfuCfuCfgAfususu |
| siRNA4162/4224 | 4162 | asuscgaGfAfAfGfuacaucccgasusu | 4224 | usCfsgGfgAfuGfuAfcUfuCfuCfgAfususu |
| siRNA4163/4224 | 4163 | asuscgaGfAfAfGfuAfcaucccgasusu | 4224 | usCfsgGfgAfuGfuAfcUfuCfuCfgAfususu |
| siRNA4164/4224 | 4164 | asuscgAfgaaGfuAfcaucccgasusu | 4224 | usCfsgGfgAfuGfuAfcUfuCfuCfgAfususu |
| siRNA4165/4224 | 4165 | asuscgAfgaAfGfuacaucccgasusu | 4224 | usCfsgGfgAfuGfuAfcUfuCfuCfgAfususu |
| siRNA4166/4224 | 4166 | asuscgAfgaAfGfuAfcaucccgasusu | 4224 | usCfsgGfgAfuGfuAfcUfuCfuCfgAfususu |
| siRNA4167/4224 | 4167 | asuscgAfgAfaGfuacaucccgasusu | 4224 | usCfsgGfgAfuGfuAfcUfuCfuCfgAfususu |
| siRNA4168/4224 | 4168 | asuscgAfgAfaGfuAfcaucccgasusu | 4224 | usCfsgGfgAfuGfuAfcUfuCfuCfgAfususu |
| siRNA4169/4224 | 4169 | asuscgAfGfaaGfuacaucccgasusu | 4224 | usCfsgGfgAfuGfuAfcUfuCfuCfgAfususu |
| siRNA4170/4224 | 4170 | asuscgAfGfaaGfuAfcaucccgasusu | 4224 | usCfsgGfgAfuGfuAfcUfuCfuCfgAfususu |
| siRNA4171/4224 | 4171 | asuscgAfGfaAfGfuacaucccgasusu | 4224 | usCfsgGfgAfuGfuAfcUfuCfuCfgAfususu |
| siRNA4172/4224 | 4172 | asuscgAfGfaAfGfuAfcaucccgasusu | 4224 | usCfsgGfgAfuGfuAfcUfuCfuCfgAfususu |
| siRNA4173/4224 | 4173 | asuscgAfGfAfAfGfuacaucccgasusu | 4224 | usCfsgGfgAfuGfuAfcUfuCfuCfgAfususu |
| siRNA4174/4225 | 4174 | uscsgagaagUfaCfaucccguasusu | 4225 | usAfscGfgGfaUfgUfaCfuUfcUfcGfasusu |
| siRNA4175/4226 | 4175 | csgsagaaGfuAfcAfucccguaasusu | 4226 | usUfsaCfgGfgAfuGfuAfcUfuCfuCfgsusu |
| siRNA4176/4226 | 4176 | csgsagaAfguAfcAfucccguaasusu | 4226 | usUfsaCfgGfgAfuGfuAfcUfuCfuCfgsusu |
| siRNA4177/4226 | 4177 | csgsagaAfGfuAfcaucccguaasusu | 4226 | usUfsaCfgGfgAfuGfuAfcUfuCfuCfgsusu |
| siRNA4178/4226 | 4178 | csgsagaAfGfuAfcAfucccguaasusu | 4226 | usUfsaCfgGfgAfuGfuAfcUfuCfuCfgsusu |
| siRNA4179/4226 | 4179 | csgsagAfaguAfcAfucccguaasusu | 4226 | usUfsaCfgGfgAfuGfuAfcUfuCfuCfgsusu |
| siRNA4180/4226 | 4180 | csgsagAfaGfuAfcaucccguaasusu | 4226 | usUfsaCfgGfgAfuGfuAfcUfuCfuCfgsusu |
| siRNA4181/4226 | 4181 | csgsagAfaGfuAfcAfucccguaasusu | 4226 | usUfsaCfgGfgAfuGfuAfcUfuCfuCfgsusu |
| siRNA4182/4226 | 4182 | csgsagAfAfguAfcaucccguaasusu | 4226 | usUfsaCfgGfgAfuGfuAfcUfuCfuCfgsusu |
| siRNA4183/4226 | 4183 | csgsagAfAfguAfcAfucccguaasusu | 4226 | usUfsaCfgGfgAfuGfuAfcUfuCfuCfgsusu |
| siRNA4184/4227 | 4184 | gsasaguaCfaUfcCfcguacacasusu | 4227 | usGfsuGfuAfcGfgGfaUfgUfaCfuUfcsusu |
| siRNA4185/4227 | 4185 | gsasaguaCfaUfCfccguacacasusu | 4227 | usGfsuGfuAfcGfgGfaUfgUfaCfuUfcsusu |
| siRNA4186/4227 | 4186 | gsasagUfacaUfcCfcguacacasusu | 4227 | usGfsuGfuAfcGfgGfaUfgUfaCfuUfcsusu |
| siRNA4187/4227 | 4187 | gsasagUfacaUfCfccguacacasusu | 4227 | usGfsuGfuAfcGfgGfaUfgUfaCfuUfcsusu |
| siRNA4188/4227 | 4188 | gsasagUfaCfaUfcccguacacasusu | 4227 | usGfsuGfuAfcGfgGfaUfgUfaCfuUfcsusu |
| siRNA4189/4227 | 4189 | gsasagUfaCfaUfcCfcguacacasusu | 4227 | usGfsuGfuAfcGfgGfaUfgUfaCfuUfcsusu |
| siRNA4190/4227 | 4190 | gsasagUfaCfaUfCfccguacacasusu | 4227 | usGfsuGfuAfcGfgGfaUfgUfaCfuUfcsusu |
| siRNA4191/4228 | 4191 | gsgsacacaAfGfuGfccagauuasusu | 4228 | usAfsaUfcUfgGfcAfcUfuGfuGfuCfcsusu |
| siRNA4192/4228 | 4192 | gsgsacacAfaGfuGfccagauuasusu | 4228 | usAfsaUfcUfgGfcAfcUfuGfuGfuCfcsusu |
| siRNA4193/4228 | 4193 | gsgsacAfcaaGfuGfccagauuasusu | 4228 | usAfsaUfcUfgGfcAfcUfuGfuGfuCfcsusu |
| siRNA4194/4228 | 4194 | gsgsacAfcaAfGfugccagauuasusu | 4228 | usAfsaUfcUfgGfcAfcUfuGfuGfuCfcsusu |
| siRNA4195/4228 | 4195 | gsgsacAfcaAfGfuGfccagauuasusu | 4228 | usAfsaUfcUfgGfcAfcUfuGfuGfuCfcsusu |
| siRNA4196/4228 | 4196 | gsgsacAfcAfaGfugccagauuasusu | 4228 | usAfsaUfcUfgGfcAfcUfuGfuGfuCfcsusu |
| siRNA4197/4228 | 4197 | gsgsacAfcAfaGfuGfccagauuasusu | 4228 | usAfsaUfcUfgGfcAfcUfuGfuGfuCfcsusu |
| siRNA4198/4229 | 4198 | ascsacaAfGfuGfccagauugcasusu | 4229 | usGfscAfaUfcUfgGfcAfcUfuGfuGfususu |
| siRNA4199/4229 | 4199 | ascsacAfaGfuGfccagauugcasusu | 4229 | usGfscAfaUfcUfgGfcAfcUfuGfuGfususu |
| siRNA4200/4229 | 4200 | ascsacAfAfguGfccagauugcasusu | 4229 | usGfscAfaUfcUfgGfcAfcUfuGfuGfususu |
| siRNA4201/4230 | 4201 | gscsuacuucGfAfcugcaaguasusu | 4230 | usAfscUfuGfcAfgUfcGfaAfgUfaGfcsusu |
| siRNA4202/4231 | 4202 | csusacuucGfAfcugcaagucasusu | 4231 | usGfsaCfuUfgCfaGfuCfgAfaGfuAfgsusu |
| siRNA4203/4232 | 4203 | ascsuucgaCfUfgCfaaguccgasusu | 4232 | usCfsgGfaCfuUfgCfaGfuCfgAfaGfususu |
| siRNA4204/4232 | 4204 | ascsuuCfgacUfgCfaaguccgasusu | 4232 | usCfsgGfaCfuUfgCfaGfuCfgAfaGfususu |
| siRNA4205/4232 | 4205 | ascsuuCfgaCfUfgcaaguccgasusu | 4232 | usCfsgGfaCfuUfgCfaGfuCfgAfaGfususu |
| siRNA4206/4232 | 4206 | ascsuuCfgaCfUfgCfaaguccgasusu | 4232 | usCfsgGfaCfuUfgCfaGfuCfgAfaGfususu |
| siRNA4207/4233 | 4207 | ususaccucuAfcGfgcaucauasusu | 4233 | usAfsuGfaUfgCfcGfuAfgAfgGfuAfasusu |
| siRNA4191/4228 | 4191 | gsgsacacaAfGfuGfccagauuasusu | 4228 | usAfsaUfcUfgGfcAfcUfuGfuGfuCfcsusu |
| siRNA4192/4228 | 4192 | gsgsacacAfaGfuGfccagauuasusu | 4228 | usAfsaUfcUfgGfcAfcUfuGfuGfuCfcsusu |
| siRNA4193/4228 | 4193 | gsgsacAfcaaGfuGfccagauuasusu | 4228 | usAfsaUfcUfgGfcAfcUfuGfuGfuCfcsusu |
| siRNA4194/4228 | 4194 | gsgsacAfcaAfGfugccagauuasusu | 4228 | usAfsaUfcUfgGfcAfcUfuGfuGfuCfcsusu |
| siRNA4195/4228 | 4195 | gsgsacAfcaAfGfuGfccagauuasusu | 4228 | usAfsaUfcUfgGfcAfcUfuGfuGfuCfcsusu |
| siRNA4196/4228 | 4196 | gsgsacAfcAfaGfugccagauuasusu | 4228 | usAfsaUfcUfgGfcAfcUfuGfuGfuCfcsusu |
| siRNA4197/4228 | 4197 | gsgsacAfcAfaGfuGfccagauuasusu | 4228 | usAfsaUfcUfgGfcAfcUfuGfuGfuCfcsusu |
| siRNA4198/4229 | 4198 | ascsacaAfGfuGfccagauugcasusu | 4229 | usGfscAfaUfcUfgGfcAfcUfuGfuGfususu |
| siRNA4199/4229 | 4199 | ascsacAfaGfuGfccagauugcasusu | 4229 | usGfscAfaUfcUfgGfcAfcUfuGfuGfususu |
| siRNA4200/4229 | 4200 | ascsacAfAfguGfccagauugcasusu | 4229 | usGfscAfaUfcUfgGfcAfcUfuGfuGfususu |
| siRNA4201/4230 | 4201 | gscsuacuucGfAfcugcaaguasusu | 4230 | usAfscUfuGfcAfgUfcGfaAfgUfaGfcsusu |
| siRNA4202/4231 | 4202 | csusacuucGfAfcugcaagucasusu | 4231 | usGfsaCfuUfgCfaGfuCfgAfaGfuAfgsusu |
| siRNA4203/4232 | 4203 | ascsuucgaCfUfgCfaaguccgasusu | 4232 | usCfsgGfaCfuUfgCfaGfuCfgAfaGfususu |
| siRNA4204/4232 | 4204 | ascsuuCfgacUfgCfaaguccgasusu | 4232 | usCfsgGfaCfuUfgCfaGfuCfgAfaGfususu |
| siRNA4205/4232 | 4205 | ascsuuCfgaCfUfgcaaguccgasusu | 4232 | usCfsgGfaCfuUfgCfaGfuCfgAfaGfususu |
| siRNA4206/4232 | 4206 | ascsuuCfgaCfUfgCfaaguccgasusu | 4232 | usCfsgGfaCfuUfgCfaGfuCfgAfaGfususu |
| siRNA4207/4233 | 4207 | ususaccucuAfcGfgcaucauasusu | 4233 | usAfsuGfaUfgCfcGfuAfgAfgGfuAfasusu |

TABLE F(2)
Subset F, Mod Screening Set with Parent SEQ ID NOS.

| siRNA name | Parent SEQ ID NO: | Parent SEQ ID NO: |
|---|---|---|
| siRNA4103/4208 | 160 | 2211 |
| siRNA4104/4208 | 160 | 2211 |
| siRNA4105/4208 | 160 | 2211 |
| siRNA4106/4208 | 160 | 2211 |
| siRNA4107/4208 | 160 | 2211 |
| siRNA4108/4208 | 160 | 2211 |
| siRNA4109/4208 | 160 | 2211 |
| siRNA4110/4208 | 160 | 2211 |
| siRNA4111/4209 | 166 | 2217 |
| siRNA4112/4209 | 166 | 2217 |
| siRNA4113/4210 | 202 | 2253 |
| siRNA4114/4210 | 202 | 2253 |
| siRNA4115/4210 | 202 | 2253 |
| siRNA4116/4211 | 203 | 2254 |
| siRNA4117/4212 | 431 | 2482 |
| siRNA4118/4213 | 434 | 2485 |
| siRNA4119/4213 | 434 | 2485 |
| siRNA4120/4214 | 436 | 2487 |
| siRNA4121/4215 | 437 | 2488 |
| siRNA4122/4215 | 437 | 2488 |
| siRNA4123/4215 | 437 | 2488 |
| siRNA4124/4216 | 438 | 2489 |
| siRNA4125/4217 | 751 | 2802 |
| siRNA4126/4217 | 751 | 2802 |
| siRNA4127/4217 | 751 | 2802 |
| siRNA4128/4217 | 751 | 2802 |
| siRNA4129/4218 | 879 | 2930 |
| siRNA4130/4219 | 1424 | 3475 |
| siRNA4131/4219 | 1424 | 3475 |
| siRNA4132/4219 | 1424 | 3475 |
| siRNA4133/4219 | 1424 | 3475 |
| siRNA4134/4219 | 1424 | 3475 |
| siRNA4135/4219 | 1424 | 3475 |
| siRNA4136/4219 | 1424 | 3475 |
| siRNA4137/4219 | 1424 | 3475 |
| siRNA4138/4219 | 1424 | 3475 |
| siRNA4139/4219 | 1424 | 3475 |
| siRNA4140/4219 | 1424 | 3475 |
| siRNA4141/4219 | 1424 | 3475 |
| siRNA4142/4219 | 1424 | 3475 |
| siRNA4143/4219 | 1424 | 3475 |
| siRNA4144/4219 | 1424 | 3475 |
| siRNA4145/4219 | 1424 | 3475 |
| siRNA4146/4219 | 1424 | 3475 |
| siRNA4147/4220 | 1428 | 3479 |
| siRNA4148/4221 | 1460 | 3511 |
| siRNA4149/4221 | 1460 | 3511 |
| siRNA4150/4221 | 1460 | 3511 |
| siRNA4151/4221 | 1460 | 3511 |
| siRNA4152/4222 | 1463 | 3514 |
| siRNA4153/4223 | 1464 | 3515 |
| siRNA4154/4223 | 1464 | 3515 |
| siRNA4155/4224 | 1469 | 3520 |
| siRNA4156/4224 | 1469 | 3520 |
| siRNA4157/4224 | 1469 | 3520 |
| siRNA4158/4224 | 1469 | 3520 |
| siRNA4159/4224 | 1469 | 3520 |
| siRNA4160/4224 | 1469 | 3520 |
| siRNA4161/4224 | 1469 | 3520 |
| siRNA4162/4224 | 1469 | 3520 |
| siRNA4163/4224 | 1469 | 3520 |
| siRNA4164/4224 | 1469 | 3520 |
| siRNA4165/4224 | 1469 | 3520 |
| siRNA4166/4224 | 1469 | 3520 |
| siRNA4167/4224 | 1469 | 3520 |
| siRNA4168/4224 | 1469 | 3520 |
| siRNA4169/4224 | 1469 | 3520 |
| siRNA4170/4224 | 1469 | 3520 |
| siRNA4171/4224 | 1469 | 3520 |
| siRNA4172/4224 | 1469 | 3520 |
| siRNA4173/4224 | 1469 | 3520 |
| siRNA4174/4225 | 1470 | 3521 |
| siRNA4175/4226 | 1471 | 3522 |
| siRNA4176/4226 | 1471 | 3522 |
| siRNA4177/4226 | 1471 | 3522 |
| siRNA4178/4226 | 1471 | 3522 |
| siRNA4179/4226 | 1471 | 3522 |
| siRNA4180/4226 | 1471 | 3522 |
| siRNA4181/4226 | 1471 | 3522 |
| siRNA4182/4226 | 1471 | 3522 |
| siRNA4183/4226 | 1471 | 3522 |
| siRNA4184/4227 | 1474 | 3525 |
| siRNA4185/4227 | 1474 | 3525 |
| siRNA4186/4227 | 1474 | 3525 |
| siRNA4187/4227 | 1474 | 3525 |
| siRNA4188/4227 | 1474 | 3525 |
| siRNA4189/4227 | 1474 | 3525 |
| siRNA4190/4227 | 1474 | 3525 |
| siRNA4191/4228 | 1628 | 3679 |
| siRNA4192/4228 | 1628 | 3679 |
| siRNA4193/4228 | 1628 | 3679 |
| siRNA4194/4228 | 1628 | 3679 |
| siRNA4195/4228 | 1628 | 3679 |
| siRNA4196/4228 | 1628 | 3679 |
| siRNA4197/4228 | 1628 | 3679 |
| siRNA4198/4229 | 1630 | 3681 |
| siRNA4199/4229 | 1630 | 3681 |
| siRNA4200/4229 | 1630 | 3681 |
| siRNA4201/4230 | 1788 | 3839 |
| siRNA4202/4231 | 1789 | 3840 |
| siRNA4203/4232 | 1791 | 3842 |
| siRNA4204/4232 | 1791 | 3842 |
| siRNA4205/4232 | 1791 | 3842 |
| siRNA4206/4232 | 1791 | 3842 |
| siRNA4207/4233 | 1864 | 3915 |
| siRNA4191/4228 | 1628 | 3679 |
| siRNA4192/4228 | 1628 | 3679 |
| siRNA4193/4228 | 1628 | 3679 |
| siRNA4194/4228 | 1628 | 3679 |
| siRNA4195/4228 | 1628 | 3679 |
| siRNA4196/4228 | 1628 | 3679 |
| siRNA4197/4228 | 1628 | 3679 |
| siRNA4198/4229 | 1630 | 3681 |
| siRNA4199/4229 | 1630 | 3681 |
| siRNA4200/4229 | 1630 | 3681 |
| siRNA4201/4230 | 1788 | 3839 |
| siRNA4202/4231 | 1789 | 3840 |
| siRNA4203/4232 | 1791 | 3842 |
| siRNA4204/4232 | 1791 | 3842 |
| siRNA4205/4232 | 1791 | 3842 |
| siRNA4206/4232 | 1791 | 3842 |
| siRNA4207/4233 | 1864 | 3915 |

Any siRNA among any of subsets A-E may comprise any modification pattern described herein. If a sequence has a different number of nucleotides in length than a modification pattern, the modification pattern may still be used with the appropriate number of additional nucleotides added 5′ or 3′ to match the number of nucleotides in the modification pattern. For example, if a sense or antisense strand of the siRNA among any of subsets A-E comprises 19 nucleotides, and a modification pattern comprises 21 nucleotides, UU may be added onto the 5′ end of the sense or antisense strand.

Therapeutic siRNAs were designed to target human HGFAC as described above and, in some cases, the HGFAC sequence of at least one toxicology-relevant species, in this case, the non-human primate (NHP) cynomolgus monkey. The siRNAs included in subset G had the following characteristics and are shown in Table G:
Cross-reactivity: With 19mer in human HGFAC mRNA, with 17mer (pos. 2-18) in NHP HGFAC
Specificity category: For human considering only those off-targets expressed in hepatocytes: AS2 or better, SS4 or better. For NHP AS2 or better, SS4 or better.
SNPs: siRNA target sites do not harbor SNPs with a MAF≥1% (pos. 2-18)

TABLE G
Subset G siRNAs

| | SEQ | Sense strand | SEQ | Antisense strand |
|---|---|---|---|---|
| | ID | sequence | ID | sequence |
| siRNA Name | NO: | (5′-3′) | NO: | (5′-3′) |
| siRNA 171 | 171 | AACCUAAUGCCACAGCGAC | 2222 | GUCGCUGUGGCAUUAGGUU |
| siRNA 203 | 203 | ACUAUCCUGGUGACCUCUG | 2254 | CAGAGGUCACCAGGAUAGU |
| siRNA 428 | 428 | UGGUGUGCCACAACUCACA | 2479 | UGUGAGUUGUGGCACACCA |
| siRNA 431 | 431 | UGUGCCACAACUCACAACU | 2482 | AGUUGUGAGUUGUGGCACA |
| siRNA 437 | 437 | ACAACUCACAACUACGACC | 2488 | GGUCGUAGUUGUGAGUUGU |
| siRNA 439 | 439 | AACUCACAACUACGACCGG | 2490 | CCGGUCGUAGUUGUGAGUU |
| siRNA 442 | 442 | UCACAACUACGACCGGGAC | 2493 | GUCCCGGUCGUAGUUGUGA |
| siRNA 1230 | 1230 | AGAAGAGGACGUUCCUGCG | 3281 | CGCAGGAACGUCCUCUUCU |
| siRNA 1425 | 1425 | AGCACUUCUUCAACCGCAC | 3476 | GUGCGGUUGAAGAAGUGCU |
| siRNA 1427 | 1427 | CACUUCUUCAACCGCACGA | 3478 | UCGUGCGGUUGAAGAAGUG |
| siRNA 1428 | 1428 | ACUUCUUCAACCGCACGAC | 3479 | GUCGUGCGGUUGAAGAAGU |
| siRNA 1429 | 1429 | CUUCUUCAACCGCACGACG | 3480 | CGUCGUGCGGUUGAAGAAG |
| siRNA 1430 | 1430 | UUCUUCAACCGCACGACGG | 3481 | CCGUCGUGCGGUUGAAGAA |
| siRNA 1431 | 1431 | UCUUCAACCGCACGACGGA | 3482 | UCCGUCGUGCGGUUGAAGA |
| siRNA 1463 | 1463 | UUCGGCAUCGAGAAGUACA | 3514 | UGUACUUCUCGAUGCCGAA |
| siRNA 1469 | 1469 | AUCGAGAAGUACAUCCCGU | 3520 | ACGGGAUGUACUUCUCGAU |
| siRNA 1470 | 1470 | UCGAGAAGUACAUCCCGUA | 3521 | UACGGGAUGUACUUCUCGA |
| siRNA 1474 | 1474 | GAAGUACAUCCCGUACACC | 3525 | GGUGUACGGGAUGUACUUC |
| siRNA 1779 | 1779 | UCUGUGCCGGCUACUUCGA | 3830 | UCGAAGUAGCCGGCACAGA |
| siRNA 1789 | 1789 | CUACUUCGACUGCAAGUCC | 3840 | GGACUUGCAGUCGAAGUAG |
| siRNA 1791 | 1791 | ACUUCGACUGCAAGUCCGA | 3842 | UCGGACUUGCAGUCGAAGU |
| siRNA 1793 | 1793 | UUCGACUGCAAGUCCGACG | 3844 | CGUCGGACUUGCAGUCGAA |
| siRNA 1864 | 1864 | UUACCUCUACGGCAUCAUC | 3915 | GAUGAUGCCGUAGAGGUAA |

In some cases, the sense strand of any of the siRNAs of subset G comprises siRNA with a particular modification pattern. In this modification pattern, position 9 counting from the 5′ end of the of the sense strand is has the 2′F modification. If position 9 of the sense strand is a pyrimidine, then all purines in the sense strand have the 2′OMe modification. If position 9 is the only pyrimidine between positions 5 and 11 of the sense stand, then position 9 is the only position with the 2′F modification in the sense strand. If position 9 and only one other base between positions 5 and 11 of the sense strand are pyrimidines, then both of these pyrimidines are the only two positions with the 2′F modification in the sense strand. If position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. If there are >2 pyrimidines between positions 5 and 11 of the sense strand, then all combinations of pyrimidines having the 2′F modification are allowed that have three to five 2′F modifications in total, provided that the sense strand does not have three 2′F modifications in a row. If position 9 of the sense strand is a purine, then all purines in the sense strand have the 2′OMe modification. If position 9 is the only purine between positions 5 and 11 of the sense stand, then position 9 is the only position with the 2′F modification in the sense strand. If position 9 and only one other base between positions 5 and 11 of the sense strand are purines, then both of these purines are the only two positions with the 2′F modification in the sense strand. If position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. If there are >2 purines between positions 5 and 11 of the sense strand, then all combinations of purines having the 2′F modification are allowed that have three to five 2′F modifications in total, provided that the sense strand does not have three 2′F modifications in a row.

In some cases, the sense strand of any of the siRNAs of subset G comprises a modification pattern which conforms to these sense strand rules. In some cases, a 2′ deoxy substitution may be used in sense strand (see, e.g., Table G(2)).

In some cases, the antisense strand of any of the siRNAs of subset G comprises modification pattern 3AS (see, e.g., Table G(2)). In some cases, a 2′ OMe substitution at position 2 of the antisense strand may be used (see, e.g., Table G(3)).

The siRNAs in subset G may comprise any other modification pattern(s).

TABLE G(2)
Modified Subset G siRNAs

| | SEQ | | SEQ | |
|---|---|---|---|---|
| | ID | Sense strand sequence | ID | Antisense strand sequence |
| siRNA Name | NO: | (5′-3′) | NO: | (5′-3′) |
| siRNA 4234/4320 | 4234 | aaccuAfAfuGfccacagcgaasusu | 4320 | usUfscGfcUfgUfgGfcAfuUfaGfgUfususu |
| siRNA 4672/4712 | 4672 | acuauccuGfGfugaccucuasusu | 4712 | usAfsgAfgGfuCfaCfcAfgGfaUfaGfususu |
| siRNA 4235/4713 | 4235 | acuaUfcCfUfdGGfugaccucuasusu | 4713 | usAfsgAfgGfuCfaCfcAfgGfaUfaGfususu |
| siRNA 4236/4321 | 4236 | uggugugCfCfaCfaacucacasusu | 4321 | usGfsuGfaGfuUfgUfgGfcAfcAfcCfasusu |
| siRNA 4237/4321 | 4237 | uggugUfgcCfaCfaacucacasusu | 4321 | usGfsuGfaGfuUfgUfgGfcAfcAfcCfasusu |
| siRNA 4238/4321 | 4238 | uggugUfgCfCfacaacucacasusu | 4321 | usGfsuGfaGfuUfgUfgGfcAfcAfcCfasusu |
| siRNA 4239/4321 | 4239 | uggugUfgCfCfaCfaacucacasusu | 4321 | usGfsuGfaGfuUfgUfgGfcAfcAfcCfasusu |
| siRNA 4673/4714 | 4673 | ugugccAfcAfAfcucacaacasusu | 4714 | usGfsuUfgUfgAfgUfuGfuGfgCfaCfasusu |
| siRNA 4674/4715 | 4674 | acaacUfCfaCfaacuacgacasusu | 4715 | usGfsuCfgUfaGfuUfgUfgAfgUfuGfususu |
| siRNA 4675/4716 | 4675 | acaaCfuCfaCfaacuacgacasusu | 4716 | usGfsuCfgUfaGfuUfgUfgAfgUfuGfususu |
| siRNA 4676/4717 | 4676 | acaaCfUfcaCfaacuacgacasusu | 4717 | usGfsuCfgUfaGfuUfgUfgAfgUfuGfususu |
| siRNA 4240/4322 | 4240 | aacucAfcAfAfcuacgaccgasusu | 4322 | usCfsgGfuCfgUfaGfuUfgUfgAfgUfususu |
| siRNA 4241/4323 | 4241 | ucacaAfcuAfcGfaccgggaasusu | 4323 | usUfscCfcGfgUfcGfuAfgUfuGfuGfasusu |
| siRNA 4242/4323 | 4242 | ucacAfacuAfcGfaccgggaasusu | 4323 | usUfscCfcGfgUfcGfuAfgUfuGfuGfasusu |
| siRNA 4243/4323 | 4243 | ucacAfAfcuAfcgaccgggaasusu | 4323 | usUfscCfcGfgUfcGfuAfgUfuGfuGfasusu |
| siRNA 4244/4323 | 4244 | ucacAfAfcuAfcGfaccgggaasusu | 4323 | usUfscCfcGfgUfcGfuAfgUfuGfuGfasusu |
| siRNA 4245/4324 | 4245 | agaagagGfAfcGfuuccugcasusu | 4324 | usGfscAfgGfaAfcGfuCfcUfcUfuCfususu |
| siRNA 4246/4324 | 4246 | agaagaGfgAfcGfuuccugcasusu | 4324 | usGfscAfgGfaAfcGfuCfcUfcUfuCfususu |
| siRNA 4247/4324 | 4247 | agaagAfggAfcGfuuccugcasusu | 4324 | usGfscAfgGfaAfcGfuCfcUfcUfuCfususu |
| siRNA 4248/4324 | 4248 | agaagAfgGfAfcguuccugcasusu | 4324 | usGfscAfgGfaAfcGfuCfcUfcUfuCfususu |
| siRNA 4249/4324 | 4249 | agaagAfgGfAfcGfuuccugcasusu | 4324 | usGfscAfgGfaAfcGfuCfcUfcUfuCfususu |
| siRNA 4250/4324 | 4250 | agaagAfGfgAfcguuccugcasusu | 4324 | usGfscAfgGfaAfcGfuCfcUfcUfuCfususu |
| siRNA 4251/4324 | 4251 | agaagAfGfgAfcGfuuccugcasusu | 4324 | usGfscAfgGfaAfcGfuCfcUfcUfuCfususu |
| siRNA 4252/4324 | 4252 | agaagAfGfGfAfcguuccugcasusu | 4324 | usGfscAfgGfaAfcGfuCfcUfcUfuCfususu |
| siRNA 4253/4324 | 4253 | agaagAfGfGfAfcGfuuccugcasusu | 4324 | usGfscAfgGfaAfcGfuCfcUfcUfuCfususu |
| siRNA 4254/4324 | 4254 | agaaGfaggAfcGfuuccugcasusu | 4324 | usGfscAfgGfaAfcGfuCfcUfcUfuCfususu |
| siRNA 4255/4324 | 4255 | agaaGfagGfAfcguuccugcasusu | 4324 | usGfscAfgGfaAfcGfuCfcUfcUfuCfususu |
| siRNA 4256/4324 | 4256 | agaaGfagGfAfcGfuuccugcasusu | 4324 | usGfscAfgGfaAfcGfuCfcUfcUfuCfususu |
| siRNA 4257/4324 | 4257 | agaaGfaGfgAfcguuccugcasusu | 4324 | usGfscAfgGfaAfcGfuCfcUfcUfuCfususu |
| siRNA 4258/4324 | 4258 | agaaGfaGfgAfcGfuuccugcasusu | 4324 | usGfscAfgGfaAfcGfuCfcUfcUfuCfususu |
| siRNA 4259/4324 | 4259 | agaaGfAfggAfcguuccugcasusu | 4324 | usGfscAfgGfaAfcGfuCfcUfcUfuCfususu |
| siRNA 4260/4324 | 4260 | agaaGfAfggAfcGfuuccugcasusu | 4324 | usGfscAfgGfaAfcGfuCfcUfcUfuCfususu |
| siRNA 4261/4324 | 4261 | agaaGfAfgGfAfcguuccugcasusu | 4324 | usGfscAfgGfaAfcGfuCfcUfcUfuCfususu |
| siRNA 4262/4324 | 4262 | agaaGfAfgGfAfcGfuuccugcasusu | 4324 | usGfscAfgGfaAfcGfuCfcUfcUfuCfususu |
| siRNA 4263/4324 | 4263 | agaaGfAfGfGfAfcguuccugcasusu | 4324 | usGfscAfgGfaAfcGfuCfcUfcUfuCfususu |
| siRNA 4264/4325 | 4264 | agcacuuCfUfuCfaaccgcaasusu | 4325 | usUfsgCfgGfuUfgAfaGfaAfgUfgCfususu |
| siRNA 4265/4325 | 4265 | agcacuuCfUfUfCfaaccgcaasusu | 4325 | usUfsgCfgGfuUfgAfaGfaAfgUfgCfususu |
| siRNA 4266/4325 | 4266 | agcacuUfcUfuCfaaccgcaasusu | 4325 | usUfsgCfgGfuUfgAfaGfaAfgUfgCfususu |
| siRNA 4267/4325 | 4267 | agcacuUfcUfUfcaaccgcaasusu | 4325 | usUfsgCfgGfuUfgAfaGfaAfgUfgCfususu |
| siRNA 4268/4325 | 4268 | agcacuUfCfUfUfcaaccgcaasusu | 4325 | usUfsgCfgGfuUfgAfaGfaAfgUfgCfususu |
| siRNA 4269/4325 | 4269 | agcacuUfCfUfUfCfaaccgcaasusu | 4325 | usUfsgCfgGfuUfgAfaGfaAfgUfgCfususu |
| siRNA 4270/4325 | 4270 | agcacUfucUfuCfaaccgcaasusu | 4325 | usUfsgCfgGfuUfgAfaGfaAfgUfgCfususu |
| siRNA 4271/4325 | 4271 | agcacUfucUfUfcaaccgcaasusu | 4325 | usUfsgCfgGfuUfgAfaGfaAfgUfgCfususu |
| siRNA 4272/4325 | 4272 | agcacUfuCfUfucaaccgcaasusu | 4325 | usUfsgCfgGfuUfgAfaGfaAfgUfgCfususu |
| siRNA 4273/4325 | 4273 | agcacUfuCfUfuCfaaccgcaasusu | 4325 | usUfsgCfgGfuUfgAfaGfaAfgUfgCfususu |
| siRNA 4274/4325 | 4274 | agcacUfuCfUfUfCfaaccgcaasusu | 4325 | usUfsgCfgGfuUfgAfaGfaAfgUfgCfususu |
| siRNA 4275/4325 | 4275 | agcacUfUfcUfucaaccgcaasusu | 4325 | usUfsgCfgGfuUfgAfaGfaAfgUfgCfususu |
| siRNA 4276/4325 | 4276 | agcacUfUfcUfuCfaaccgcaasusu | 4325 | usUfsgCfgGfuUfgAfaGfaAfgUfgCfususu |
| siRNA 4277/4325 | 4277 | agcacUfUfcUfUfcaaccgcaasusu | 4325 | usUfsgCfgGfuUfgAfaGfaAfgUfgCfususu |
| siRNA 4278/4325 | 4278 | agcacUfUfCfUfucaaccgcaasusu | 4325 | usUfsgCfgGfuUfgAfaGfaAfgUfgCfususu |
| siRNA 4279/4325 | 4279 | agcacUfUfCfUfuCfaaccgcaasusu | 4325 | usUfsgCfgGfuUfgAfaGfaAfgUfgCfususu |
| siRNA 4280/4325 | 4280 | agcacUfUfCfUfUfcaaccgcaasusu | 4325 | usUfsgCfgGfuUfgAfaGfaAfgUfgCfususu |
| siRNA 4281/4325 | 4281 | agcaCfuucUfuCfaaccgcaasusu | 4325 | usUfsgCfgGfuUfgAfaGfaAfgUfgCfususu |
| siRNA 4282/4325 | 4282 | agcaCfuucUfUfcaaccgcaasusu | 4325 | usUfsgCfgGfuUfgAfaGfaAfgUfgCfususu |
| siRNA 4283/4325 | 4283 | agcaCfuuCfUfucaaccgcaasusu | 4325 | usUfsgCfgGfuUfgAfaGfaAfgUfgCfususu |
| siRNA 4284/4325 | 4284 | agcaCfuuCfUfuCfaaccgcaasusu | 4325 | usUfsgCfgGfuUfgAfaGfaAfgUfgCfususu |
| siRNA 4285/4325 | 4285 | agcaCfuuCfUfUfCfaaccgcaasusu | 4325 | usUfsgCfgGfuUfgAfaGfaAfgUfgCfususu |
| siRNA 4286/4325 | 4286 | agcaCfuUfcUfucaaccgcaasusu | 4325 | usUfsgCfgGfuUfgAfaGfaAfgUfgCfususu |
| siRNA 4287/4325 | 4287 | agcaCfuUfcUfuCfaaccgcaasusu | 4325 | usUfsgCfgGfuUfgAfaGfaAfgUfgCfususu |
| siRNA 4288/4325 | 4288 | agcaCfuUfcUfUfcaacegcaasusu | 4325 | usUfsgCfgGfuUfgAfaGfaAfgUfgCfususu |
| siRNA 4289/4325 | 4289 | agcaCfuUfCfUfUfcaaccgcaasusu | 4325 | usUfsgCfgGfuUfgAfaGfaAfgUfgCfususu |
| siRNA 4290/4325 | 4290 | agcaCfUfucUfucaaccgcaasusu | 4325 | usUfsgCfgGfuUfgAfaGfaAfgUfgCfususu |
| siRNA 4291/4325 | 4291 | agcaCfUfucUfuCfaaccgcaasusu | 4325 | usUfsgCfgGfuUfgAfaGfaAfgUfgCfususu |
| siRNA 4292/4325 | 4292 | agcaCfUfucUfUfcaaccgcaasusu | 4325 | usUfsgCfgGfuUfgAfaGfaAfgUfgCfususu |
| siRNA 4293/4325 | 4293 | agcaCfUfuCfUfucaaccgcaasusu | 4325 | usUfsgCfgGfuUfgAfaGfaAfgUfgCfususu |
| siRNA 4294/4325 | 4294 | agcaCfUfuCfUfuCfaaccgcaasusu | 4325 | usUfsgCfgGfuUfgAfaGfaAfgUfgCfususu |
| siRNA 4295/4325 | 4295 | agcaCfUfUfCfUfucaaccgcaasusu | 4325 | usUfsgCfgGfuUfgAfaGfaAfgUfgCfususu |
| siRNA 4296/4326 | 4296 | cacuuCfuUfCfaaccgcacgasusu | 4326 | usCfsgUfgCfgGfuUfgAfaGfaAfgUfgsusu |
| siRNA 4297/4326 | 4297 | cacuuCfUfuCfaaccgcacgasusu | 4326 | usCfsgUfgCfgGfuUfgAfaGfaAfgUfgsusu |
| siRNA 4298/4326 | 4298 | cacuuCfUfUfCfaaccgcacgasusu | 4326 | usCfsgUfgCfgGfuUfgAfaGfaAfgUfgsusu |
| siRNA 4299/4326 | 4299 | cacuUfcuUfCfaaccgcacgasusu | 4326 | usCfsgUfgCfgGfuUfgAfaGfaAfgUfgsusu |
| siRNA 4300/4326 | 4300 | cacuUfcUfuCfaaccgcacgasusu | 4326 | usCfsgUfgCfgGfuUfgAfaGfaAfgUfgsusu |
| siRNA 4301/4326 | 4301 | cacuUfCfuuCfaaccgcacgasusu | 4326 | usCfsgUfgCfgGfuUfgAfaGfaAfgUfgsusu |
| siRNA 4302/4326 | 4302 | cacuUfCfuUfCfaaccgcacgasusu | 4326 | usCfsgUfgCfgGfuUfgAfaGfaAfgUfgsusu |
| siRNA 4303/4326 | 4303 | cacuUfCfUfUfCfaaccgcacgasusu | 4326 | usCfsgUfgCfgGfuUfgAfaGfaAfgUfgsusu |
| siRNA 4677/4718 | 4677 | acuucuucAfAfccgcacgaasusu | 4718 | usUfscGfuGfcGfgUfuGfaAfgAfaGfususu |
| siRNA 4304/4719 | 4304 | acuuCfuUfCfdAAfccgcacgaasusu | 4719 | usUfscGfuGfcGfgUfuGfaAfgAfaGfususu |
| siRNA 4305/4327 | 4305 | cuucuucAfAfccgcacgacasusu | 4327 | usGfsuCfgUfgCfgGfuUfgAfaGfaAfgsusu |
| siRNA 4306/4327 | 4306 | cuucUfuCfAfdAccgcacgacasusu | 4327 | usGfsuCfgUfgCfgGfuUfgAfaGfaAfgsusu |
| siRNA 4307/4328 | 4307 | uucuuCfaaCfCfgcacgacgasusu | 4328 | usCfsgUfcGfuGfcGfgUfuGfaAfgAfasusu |
| siRNA 4308/4328 | 4308 | uucuUfcaaCfCfgcacgacgasusu | 4328 | usCfsgUfcGfuGfcGfgUfuGfaAfgAfasusu |
| siRNA 4309/4328 | 4309 | uucuUfCfaaCfcgcacgacgasusu | 4328 | usCfsgUfcGfuGfcGfgUfuGfaAfgAfasusu |
| siRNA 4310/4328 | 4310 | uucuUfCfaaCfCfgcacgacgasusu | 4328 | usCfsgUfcGfuGfcGfgUfuGfaAfgAfasusu |
| siRNA 4311/4329 | 4311 | ucuucaaCfCfgCfacgacggasusu | 4329 | usCfscGfuCfgUfgCfgGfuUfgAfaGfasusu |
| siRNA 4312/4329 | 4312 | ucuuCfaacCfgCfacgacggasusu | 4329 | usCfscGfuCfgUfgCfgGfuUfgAfaGfasusu |
| siRNA 4313/4329 | 4313 | ucuuCfaaCfCfgcacgacggasusu | 4329 | usCfscGfuCfgUfgCfgGfuUfgAfaGfasusu |
| siRNA 4314/4329 | 4314 | ucuuCfaaCfCfgCfacgacggasusu | 4329 | usCfscGfuCfgUfgCfgGfuUfgAfaGfasusu |
| siRNA 4678/4720 | 4678 | uucggCfaUfCfgagaaguacasusu | 4720 | usGfsuAfcUfuCfuCfgAfuGfcCfgAfasusu |
| siRNA 4679/4721 | 4679 | aucgagaAfGfuAfcaucccgasusu | 4721 | usCfsgGfgAfuGfuAfcUfuCfuCfgAfususu |
| siRNA 4680/4722 | 4680 | aucgagAfaGfuAfcaucccgasusu | 4722 | usCfsgGfgAfuGfuAfcUfuCfuCfgAfususu |
| siRNA 4681/4723 | 4681 | aucgaGfaaGfuAfcaucccgasusu | 4723 | usCfsgGfgAfuGfuAfcUfuCfuCfgAfususu |
| siRNA 4682/4724 | 4682 | aucgaGfaAfGfuacaucccgasusu | 4724 | usCfsgGfgAfuGfuAfcUfuCfuCfgAfususu |
| siRNA 4683/4725 | 4683 | aucgaGfaAfGfuAfcaucccgasusu | 4725 | usCfsgGfgAfuGfuAfcUfuCfuCfgAfususu |
| siRNA 4684/4726 | 4684 | aucgaGfAfaGfuacaucccgasusu | 4726 | usCfsgGfgAfuGfuAfcUfuCfuCfgAfususu |
| siRNA 4685/4727 | 4685 | aucgaGfAfaGfuAfcaucccgasusu | 4727 | usCfsgGfgAfuGfuAfcUfuCfuCfgAfususu |
| siRNA 4686/4728 | 4686 | aucgaGfAfAfGfuacaucccgasusu | 4728 | usCfsgGfgAfuGfuAfcUfuCfuCfgAfususu |
| siRNA 4687/4729 | 4687 | aucgaGfAfAfGfuAfcaucccgasusu | 4729 | usCfsgGfgAfuGfuAfcUfuCfuCfgAfususu |
| siRNA 4688/4730 | 4688 | aucgAfgaaGfuAfcaucccgasusu | 4730 | usCfsgGfgAfuGfuAfcUfuCfuCfgAfususu |
| siRNA 4689/4731 | 4689 | aucgAfgaAfGfuacaucccgasusu | 4731 | usCfsgGfgAfuGfuAfcUfuCfuCfgAfususu |
| siRNA 4690/4732 | 4690 | aucgAfgaAfGfuAfcaucccgasusu | 4732 | usCfsgGfgAfuGfuAfcUfuCfuCfgAfususu |
| siRNA 4691/4733 | 4691 | aucgAfgAfaGfuacaucccgasusu | 4733 | usCfsgGfgAfuGfuAfcUfuCfuCfgAfususu |
| siRNA 4692/4734 | 4692 | aucgAfgAfaGfuAfcaucccgasusu | 4734 | usCfsgGfgAfuGfuAfcUfuCfuCfgAfususu |
| siRNA 4693/4735 | 4693 | aucgAfGfaaGfuacaucccgasusu | 4735 | usCfsgGfgAfuGfuAfcUfuCfuCfgAfususu |
| siRNA 4694/4736 | 4694 | aucgAfGfaaGfuAfcaucccgasusu | 4736 | usCfsgGfgAfuGfuAfcUfuCfuCfgAfususu |
| siRNA 4695/4737 | 4695 | aucgAfGfaAfGfuacaucccgasusu | 4737 | usCfsgGfgAfuGfuAfcUfuCfuCfgAfususu |
| siRNA 4696/4738 | 4696 | aucgAfGfaAfGfuAfcaucccgasusu | 4738 | usCfsgGfgAfuGfuAfcUfuCfuCfgAfususu |
| siRNA 4697/4739 | 4697 | aucgAfGfAfAfGfuacaucccgasusu | 4739 | usCfsgGfgAfuGfuAfcUfuCfuCfgAfususu |
| siRNA 4698/4740 | 4698 | ucgagaagUfaCfaucccguasusu | 4740 | usAfscGfgGfaUfgUfaCfuUfcUfcGfasusu |
| siRNA 4315/4741 | 4315 | ucgaGfaAfGfdTaCfaucccguasusu | 4741 | usAfscGfgGfaUfgUfaCfuUfcUfcGfasusu |
| siRNA 4699/4742 | 4699 | gaaguaCfaUfcCfcguacacasusu | 4742 | usGfsuGfuAfcGfgGfaUfgUfaCfuUfcsusu |
| siRNA 4700/4743 | 4700 | gaaguaCfaUfCfccguacacasusu | 4743 | usGfsuGfuAfcGfgGfaUfgUfaCfuUfcsusu |
| siRNA 4701/4744 | 4701 | gaagUfacaUfcCfcguacacasusu | 4744 | usGfsuGfuAfcGfgGfaUfgUfaCfuUfcsusu |
| siRNA 4702/4745 | 4702 | gaagUfacaUfCfccguacacasusu | 4745 | usGfsuGfuAfcGfgGfaUfgUfaCfuUfcsusu |
| siRNA 4703/4746 | 4703 | gaagUfaCfaUfcccguacacasusu | 4746 | usGfsuGfuAfcGfgGfaUfgUfaCfuUfcsusu |
| siRNA 4704/4747 | 4704 | gaagUfaCfaUfcCfcguacacasusu | 4747 | usGfsuGfuAfcGfgGfaUfgUfaCfuUfcsusu |
| siRNA 4705/4748 | 4705 | gaagUfaCfaUfCfccguacacasusu | 4748 | usGfsuGfuAfcGfgGfaUfgUfaCfuUfcsusu |
| siRNA 4316/4330 | 4316 | ucuguGfccGfGfcuacuucgasusu | 4330 | usCfsgAfaGfuAfgCfcGfgCfaCfaGfasusu |
| siRNA 4706/4749 | 4706 | cuacuucGfAfcugcaagucasusu | 4749 | usGfsaCfuUfgCfaGfuCfgAfaGfuAfgsusu |
| siRNA 4317/4750 | 4317 | cuacUfuCfGfdAcugcaagucasusu | 4750 | usGfsaCfuUfgCfaGfuCfgAfaGfuAfgsusu |
| siRNA 4707/4751 | 4707 | acuucgaCfUfgCfaaguccgasusu | 4751 | usCfsgGfaCfuUfgCfaGfuCfgAfaGfususu |
| siRNA 4708/4752 | 4708 | acuuCfgacUfgCfaaguccgasusu | 4752 | usCfsgGfaCfuUfgCfaGfuCfgAfaGfususu |
| siRNA 4709/4753 | 4709 | acuuCfgaCfUfgcaaguccgasusu | 4753 | usCfsgGfaCfuUfgCfaGfuCfgAfaGfususu |
| siRNA 4710/4754 | 4710 | acuuCfgaCfUfgCfaaguccgasusu | 4754 | usCfsgGfaCfuUfgCfaGfuCfgAfaGfususu |
| siRNA 4318/4331 | 4318 | uucgaCfUfgCfaaguccgacasusu | 4331 | usGfsuCfgGfaCfuUfgCfaGfuCfgAfasusu |
| siRNA 4711/4755 | 4711 | uuaccucuAfcGfgcaucauasusu | 4755 | usAfsuGfaUfgCfcGfuAfgAfgGfuAfasusu |
| siRNA 4319/4756 | 4319 | uuacCfuCfUfdAcGfgcaucauasusu | 4756 | usAfsuGfaUfgCfcGfuAfgAfgGfuAfasusu |

TABLE G(3)
Modified Subset G siRNAs with a 2′OMe Substitution at Position 2 of the AS Strand

| | SEQ | | SEQ | |
|---|---|---|---|---|
| | ID | Sense strand sequence | ID | Antisense strand sequence |
| siRNA Name | NO: | (5′-3′) | NO: | (5′-3′) |
| siRNA 4234/2222 | 4234 | aaccuAfAfuGfccacagcgaasusu | 4332 | uucGfcUfgUfgGfcAfuUfaGfgUfususu |
| siRNA 4672/4333 | 4672 | acuauccuGfGfugaccucuasusu | 4333 | uagAfgGfuCfaCfcAfgGfaUfaGfususu |
| siRNA 4235/4333 | 4235 | acuaUfcCfUfdGGfugaccucuasusu | 4333 | uagAfgGfuCfaCfcAfgGfaUfaGfususu |
| siRNA 4236/4334 | 4236 | uggugugCfCfaCfaacucacasusu | 4334 | uguGfaGfuUfgUfgGfcAfcAfcCfasusu |
| siRNA 4237/4334 | 4237 | uggugUfgcCfaCfaacucacasusu | 4334 | uguGfaGfuUfgUfgGfcAfcAfcCfasusu |
| siRNA 4238/4334 | 4238 | uggugUfgCfCfacaacucacasusu | 4334 | uguGfaGfuUfgUfgGfcAfcAfcCfasusu |
| siRNA 4239/4334 | 4239 | uggugUfgCfCfaCfaacucacasusu | 4334 | uguGfaGfuUfgUfgGfcAfcAfcCfasusu |
| siRNA 4673/4335 | 4673 | ugugccAfcAfAfcucacaacasusu | 4335 | uguUfgUfgAfgUfuGfuGfgCfaCfasusu |
| siRNA 4674/4336 | 4674 | acaacUfCfaCfaacuacgacasusu | 4336 | uguCfgUfaGfuUfgUfgAfgUfuGfususu |
| siRNA 4675/4336 | 4675 | acaaCfuCfaCfaacuacgacasusu | 4336 | uguCfgUfaGfuUfgUfgAfgUfuGfususu |
| siRNA 4676/4336 | 4676 | acaaCfUfcaCfaacuacgacasusu | 4336 | uguCfgUfaGfuUfgUfgAfgUfuGfususu |
| siRNA 4240/4337 | 4240 | aacucAfcAfAfcuacgaccgasusu | 4337 | ucgGfuCfgUfaGfuUfgUfgAfgUfususu |
| siRNA 4241/4338 | 4241 | ucacaAfcuAfcGfaccgggaasusu | 4338 | uucCfcGfgUfcGfuAfgUfuGfuGfasusu |
| siRNA 4242/4338 | 4242 | ucacAfacuAfcGfaccgggaasusu | 4338 | uucCfcGfgUfcGfuAfgUfuGfuGfasusu |
| siRNA 4243/4338 | 4243 | ucacAfAfcuAfcgaccgggaasusu | 4338 | uucCfcGfgUfcGfuAfgUfuGfuGfasusu |
| siRNA 4244/4338 | 4244 | ucacAfAfcuAfcGfaccgggaasusu | 4338 | uucCfcGfgUfcGfuAfgUfuGfuGfasusu |
| siRNA 4245/4339 | 4245 | agaagagGfAfcGfuuccugcasusu | 4339 | ugcAfgGfaAfcGfuCfcUfcUfuCfususu |
| siRNA 4246/4339 | 4246 | agaagaGfgAfcGfuuccugcasusu | 4339 | ugcAfgGfaAfcGfuCfcUfcUfuCfususu |
| siRNA 4247/4339 | 4247 | agaagAfggAfcGfuuccugcasusu | 4339 | ugcAfgGfaAfcGfuCfcUfcUfuCfususu |
| siRNA 4248/4339 | 4248 | agaagAfgGfAfcguuccugcasusu | 4339 | ugcAfgGfaAfcGfuCfcUfcUfuCfususu |
| siRNA 4249/4339 | 4249 | agaagAfgGfAfcGfuuccugcasusu | 4339 | ugcAfgGfaAfcGfuCfcUfcUfuCfususu |
| siRNA 4250/4339 | 4250 | agaagAfGfgAfcguuccugcasusu | 4339 | ugcAfgGfaAfcGfuCfcUfcUfuCfususu |
| siRNA 4251/4339 | 4251 | agaagAfGfgAfcGfuuccugcasusu | 4339 | ugcAfgGfaAfcGfuCfcUfcUfuCfususu |
| siRNA 4252/4339 | 4252 | agaagAfGfGfAfcguuccugcasusu | 4339 | ugcAfgGfaAfcGfuCfcUfcUfuCfususu |
| siRNA 4253/4339 | 4253 | agaagAfGfGfAfcGfuuccugcasusu | 4339 | ugcAfgGfaAfcGfuCfcUfcUfuCfususu |
| siRNA 4254/4339 | 4254 | agaaGfaggAfcGfuuccugcasusu | 4339 | ugcAfgGfaAfcGfuCfcUfcUfuCfususu |
| siRNA 4255/4339 | 4255 | agaaGfagGfAfcguuccugcasusu | 4339 | ugcAfgGfaAfcGfuCfcUfcUfuCfususu |
| siRNA 4256/4339 | 4256 | agaaGfagGfAfcGfuuccugcasusu | 4339 | ugcAfgGfaAfcGfuCfcUfcUfuCfususu |
| siRNA 4257/4339 | 4257 | agaaGfaGfgAfcguuccugcasusu | 4339 | ugcAfgGfaAfcGfuCfcUfcUfuCfususu |
| siRNA 4258/4339 | 4258 | agaaGfaGfgAfcGfuuccugcasusu | 4339 | ugcAfgGfaAfcGfuCfcUfcUfuCfususu |
| siRNA 4259/4339 | 4259 | agaaGfAfggAfcguuccugcasusu | 4339 | ugcAfgGfaAfcGfuCfcUfcUfuCfususu |
| siRNA 4260/4339 | 4260 | agaaGfAfggAfcGfuuccugcasusu | 4339 | ugcAfgGfaAfcGfuCfcUfcUfuCfususu |
| siRNA 4261/4339 | 4261 | agaaGfAfgGfAfcguuccugcasusu | 4339 | ugcAfgGfaAfcGfuCfcUfcUfuCfususu |
| siRNA 4262/4339 | 4262 | agaaGfAfgGfAfcGfuuccugcasusu | 4339 | ugcAfgGfaAfcGfuCfcUfcUfuCfususu |
| siRNA 4263/4339 | 4263 | agaaGfAfGfGfAfcguuccugcasusu | 4339 | ugcAfgGfaAfcGfuCfcUfcUfuCfususu |
| siRNA 4264/4340 | 4264 | agcacuuCfUfuCfaaccgcaasusu | 4340 | uugCfgGfuUfgAfaGfaAfgUfgCfususu |
| siRNA 4265/4340 | 4265 | agcacuuCfUfUfCfaaccgcaasusu | 4340 | uugCfgGfuUfgAfaGfaAfgUfgCfususu |
| siRNA 4266/4340 | 4266 | agcacuUfcUfuCfaaccgcaasusu | 4340 | uugCfgGfuUfgAfaGfaAfgUfgCfususu |
| siRNA 4267/4340 | 4267 | agcacuUfcUfUfcaaccgcaasusu | 4340 | uugCfgGfuUfgAfaGfaAfgUfgCfususu |
| siRNA 4268/4340 | 4268 | agcacuUfCfUfUfcaaccgcaasusu | 4340 | uugCfgGfuUfgAfaGfaAfgUfgCfususu |
| siRNA 4269/4340 | 4269 | agcacuUfCfUfUfCfaaccgcaasusu | 4340 | uugCfgGfuUfgAfaGfaAfgUfgCfususu |
| siRNA 4270/4340 | 4270 | agcacUfucUfuCfaaccgcaasusu | 4340 | uugCfgGfuUfgAfaGfaAfgUfgCfususu |
| siRNA 4271/4340 | 4271 | agcacUfucUfUfcaaccgcaasusu | 4340 | uugCfgGfuUfgAfaGfaAfgUfgCfususu |
| siRNA 4272/4340 | 4272 | agcacUfuCfUfucaaccgcaasusu | 4340 | uugCfgGfuUfgAfaGfaAfgUfgCfususu |
| siRNA 4273/4340 | 4273 | agcacUfuCfUfuCfaaccgcaasusu | 4340 | uugCfgGfuUfgAfaGfaAfgUfgCfususu |
| siRNA 4274/4340 | 4274 | agcacUfuCfUfUfCfaaccgcaasusu | 4340 | uugCfgGfuUfgAfaGfaAfgUfgCfususu |
| siRNA 4275/4340 | 4275 | agcacUfUfcUfucaaccgcaasusu | 4340 | uugCfgGfuUfgAfaGfaAfgUfgCfususu |
| siRNA 4276/4340 | 4276 | agcacUfUfcUfuCfaaccgcaasusu | 4340 | uugCfgGfuUfgAfaGfaAfgUfgCfususu |
| siRNA 4277/4340 | 4277 | agcacUfUfcUfUfcaaccgcaasusu | 4340 | uugCfgGfuUfgAfaGfaAfgUfgCfususu |
| siRNA 4278/4340 | 4278 | agcacUfUfCfUfucaaccgcaasusu | 4340 | uugCfgGfuUfgAfaGfaAfgUfgCfususu |
| siRNA 4279/4340 | 4279 | agcacUfUfCfUfuCfaaccgcaasusu | 4340 | uugCfgGfuUfgAfaGfaAfgUfgCfususu |
| siRNA 4280/4340 | 4280 | agcacUfUfCfUfUfcaaccgcaasusu | 4340 | uugCfgGfuUfgAfaGfaAfgUfgCfususu |
| siRNA 4281/4340 | 4281 | agcaCfuucUfuCfaaccgcaasusu | 4340 | uugCfgGfuUfgAfaGfaAfgUfgCfususu |
| siRNA 4282/4340 | 4282 | agcaCfuucUfUfcaaccgcaasusu | 4340 | uugCfgGfuUfgAfaGfaAfgUfgCfususu |
| siRNA 4283/4340 | 4283 | agcaCfuuCfUfucaaccgcaasusu | 4340 | uugCfgGfuUfgAfaGfaAfgUfgCfususu |
| siRNA 4284/4340 | 4284 | agcaCfuuCfUfuCfaaccgcaasusu | 4340 | uugCfgGfuUfgAfaGfaAfgUfgCfususu |
| siRNA 4285/4340 | 4285 | agcaCfuuCfUfUfCfaaccgcaasusu | 4340 | uugCfgGfuUfgAfaGfaAfgUfgCfususu |
| siRNA 4286/4340 | 4286 | agcaCfuUfcUfucaaccgcaasusu | 4340 | uugCfgGfuUfgAfaGfaAfgUfgCfususu |
| siRNA 4287/4340 | 4287 | agcaCfuUfcUfuCfaaccgcaasusu | 4340 | uugCfgGfuUfgAfaGfaAfgUfgCfususu |
| siRNA 4288/4340 | 4288 | agcaCfuUfcUfUfcaaccgcaasusu | 4340 | uugCfgGfuUfgAfaGfaAfgUfgCfususu |
| siRNA 4289/4340 | 4289 | agcaCfuUfCfUfUfcaaccgcaasusu | 4340 | uugCfgGfuUfgAfaGfaAfgUfgCfususu |
| siRNA 4290/4340 | 4290 | agcaCfUfucUfucaaccgcaasusu | 4340 | uugCfgGfuUfgAfaGfaAfgUfgCfususu |
| siRNA 4291/4340 | 4291 | agcaCfUfucUfuCfaaccgcaasusu | 4340 | uugCfgGfuUfgAfaGfaAfgUfgCfususu |
| siRNA 4292/4340 | 4292 | agcaCfUfucUfUfcaaccgcaasusu | 4340 | uugCfgGfuUfgAfaGfaAfgUfgCfususu |
| siRNA 4293/4340 | 4293 | agcaCfUfuCfUfucaaccgcaasusu | 4340 | uugCfgGfuUfgAfaGfaAfgUfgCfususu |
| siRNA 4294/4340 | 4294 | agcaCfUfuCfUfuCfaaccgcaasusu | 4340 | uugCfgGfuUfgAfaGfaAfgUfgCfususu |
| siRNA 4295/4340 | 4295 | agcaCfUfUfCfUfucaaccgcaasusu | 4340 | uugCfgGfuUfgAfaGfaAfgUfgCfususu |
| siRNA 4296/4341 | 4296 | cacuuCfuUfCfaaccgcacgasusu | 4341 | ucgUfgCfgGfuUfgAfaGfaAfgUfgsusu |
| siRNA 4297/4341 | 4297 | cacuuCfUfuCfaaccgcacgasusu | 4341 | ucgUfgCfgGfuUfgAfaGfaAfgUfgsusu |
| siRNA 4298/4341 | 4298 | cacuuCfUfUfCfaaccgcacgasusu | 4341 | ucgUfgCfgGfuUfgAfaGfaAfgUfgsusu |
| siRNA 4299/4341 | 4299 | cacuUfcuUfCfaaccgcacgasusu | 4341 | ucgUfgCfgGfuUfgAfaGfaAfgUfgsusu |
| siRNA 4300/4341 | 4300 | cacuUfcUfuCfaaccgcacgasusu | 4341 | ucgUfgCfgGfuUfgAfaGfaAfgUfgsusu |
| siRNA 4301/4341 | 4301 | cacuUfCfuuCfaaccgcacgasusu | 4341 | ucgUfgCfgGfuUfgAfaGfaAfgUfgsusu |
| siRNA 4302/4341 | 4302 | cacuUfCfuUfCfaaccgcacgasusu | 4341 | ucgUfgCfgGfuUfgAfaGfaAfgUfgsusu |
| siRNA 4303/4341 | 4303 | cacuUfCfUfUfCfaaccgcacgasusu | 4341 | ucgUfgCfgGfuUfgAfaGfaAfgUfgsusu |
| siRNA 4677/4342 | 4677 | acuucuucAfAfccgcacgaasusu | 4342 | uucGfuGfcGfgUfuGfaAfgAfaGfususu |
| siRNA 4304/4342 | 4304 | acuuCfuUfCfdAAfccgcacgaasusu | 4342 | uucGfuGfcGfgUfuGfaAfgAfaGfususu |
| siRNA 4305/4343 | 4305 | cuucuucAfAfccgcacgacasusu | 4343 | uguCfgUfgCfgGfuUfgAfaGfaAfgsusu |
| siRNA 4306/4343 | 4306 | cuucUfuCfAfdAccgcacgacasusu | 4343 | uguCfgUfgCfgGfuUfgAfaGfaAfgsusu |
| siRNA 4307/4344 | 4307 | uucuuCfaaCfCfgcacgacgasusu | 4344 | ucgUfcGfuGfcGfgUfuGfaAfgAfasusu |
| siRNA 4308/4344 | 4308 | uucuUfcaaCfCfgcacgacgasusu | 4344 | ucgUfcGfuGfcGfgUfuGfaAfgAfasusu |
| siRNA 4309/4344 | 4309 | uucuUfCfaaCfcgcacgacgasusu | 4344 | ucgUfcGfuGfcGfgUfuGfaAfgAfasusu |
| siRNA 4310/4344 | 4310 | uucuUfCfaaCfCfgcacgacgasusu | 4344 | ucgUfcGfuGfcGfgUfuGfaAfgAfasusu |
| siRNA 4311/4345 | 4311 | ucuucaaCfCfgCfacgacggasusu | 4345 | uccGfuCfgUfgCfgGfuUfgAfaGfasusu |
| siRNA 4312/4345 | 4312 | ucuuCfaacCfgCfacgacggasusu | 4345 | uccGfuCfgUfgCfgGfuUfgAfaGfasusu |
| siRNA 4313/4345 | 4313 | ucuuCfaaCfCfgcacgacggasusu | 4345 | uccGfuCfgUfgCfgGfuUfgAfaGfasusu |
| siRNA 4314/4345 | 4314 | ucuuCfaaCfCfgCfacgacggasusu | 4345 | uccGfuCfgUfgCfgGfuUfgAfaGfasusu |
| siRNA 4678/4346 | 4678 | uucggCfaUfCfgagaaguacasusu | 4346 | uguAfcUfuCfuCfgAfuGfcCfgAfasusu |
| siRNA 4679/4347 | 4679 | aucgagaAfGfuAfcaucccgasusu | 4347 | ucgGfgAfuGfuAfcUfuCfuCfgAfususu |
| siRNA 4680/4347 | 4680 | aucgagAfaGfuAfcaucccgasusu | 4347 | ucgGfgAfuGfuAfcUfuCfuCfgAfususu |
| siRNA 4681/4347 | 4681 | aucgaGfaaGfuAfcaucccgasusu | 4347 | ucgGfgAfuGfuAfcUfuCfuCfgAfususu |
| siRNA 4682/4347 | 4682 | aucgaGfaAfGfuacaucccgasusu | 4347 | ucgGfgAfuGfuAfcUfuCfuCfgAfususu |
| siRNA 4683/4347 | 4683 | aucgaGfaAfGfuAfcaucccgasusu | 4347 | ucgGfgAfuGfuAfcUfuCfuCfgAfususu |
| siRNA 4684/4347 | 4684 | aucgaGfAfaGfuacaucccgasusu | 4347 | ucgGfgAfuGfuAfcUfuCfuCfgAfususu |
| siRNA 4685/4347 | 4685 | aucgaGfAfaGfuAfcaucccgasusu | 4347 | ucgGfgAfuGfuAfcUfuCfuCfgAfususu |
| siRNA 4686/4347 | 4686 | aucgaGfAfAfGfuacaucccgasusu | 4347 | ucgGfgAfuGfuAfcUfuCfuCfgAfususu |
| siRNA 4687/4347 | 4687 | aucgaGfAfAfGfuAfcaucccgasusu | 4347 | ucgGfgAfuGfuAfcUfuCfuCfgAfususu |
| siRNA 4688/4347 | 4688 | aucgAfgaaGfuAfcaucccgasusu | 4347 | ucgGfgAfuGfuAfcUfuCfuCfgAfususu |
| siRNA 4689/4347 | 4689 | aucgAfgaAfGfuacaucccgasusu | 4347 | ucgGfgAfuGfuAfcUfuCfuCfgAfususu |
| siRNA 4690/4347 | 4690 | aucgAfgaAfGfuAfcaucccgasusu | 4347 | ucgGfgAfuGfuAfcUfuCfuCfgAfususu |
| siRNA 4691/4347 | 4691 | aucgAfgAfaGfuacaucccgasusu | 4347 | ucgGfgAfuGfuAfcUfuCfuCfgAfususu |
| siRNA 4692/4347 | 4692 | aucgAfgAfaGfuAfcaucccgasusu | 4347 | ucgGfgAfuGfuAfcUfuCfuCfgAfususu |
| siRNA 4693/4347 | 4693 | aucgAfGfaaGfuacaucccgasusu | 4347 | ucgGfgAfuGfuAfcUfuCfuCfgAfususu |
| siRNA 4694/4347 | 4694 | aucgAfGfaaGfuAfcaucccgasusu | 4347 | ucgGfgAfuGfuAfcUfuCfuCfgAfususu |
| siRNA 4695/4347 | 4695 | aucgAfGfaAfGfuacaucccgasusu | 4347 | ucgGfgAfuGfuAfcUfuCfuCfgAfususu |
| siRNA 4696/4347 | 4696 | aucgAfGfaAfGfuAfcaucccgasusu | 4347 | ucgGfgAfuGfuAfcUfuCfuCfgAfususu |
| siRNA 4697/4347 | 4697 | aucgAfGfAfAfGfuacaucccgasusu | 4347 | ucgGfgAfuGfuAfcUfuCfuCfgAfususu |
| siRNA 4698/4348 | 4698 | ucgagaagUfaCfaucccguasusu | 4348 | uacGfgGfaUfgUfaCfuUfcUfcGfasusu |
| siRNA 4315/4348 | 4315 | ucgaGfaAfGfdTaCfaucccguasusu | 4348 | uacGfgGfaUfgUfaCfuUfcUfcGfasusu |
| siRNA 4699/4349 | 4699 | gaaguaCfaUfcCfcguacacasusu | 4349 | uguGfuAfcGfgGfaUfgUfaCfuUfcsusu |
| siRNA 4700/4349 | 4700 | gaaguaCfaUfCfccguacacasusu | 4349 | uguGfuAfcGfgGfaUfgUfaCfuUfcsusu |
| siRNA 4701/4349 | 4701 | gaagUfacaUfcCfcguacacasusu | 4349 | uguGfuAfcGfgGfaUfgUfaCfuUfcsusu |
| siRNA 4702/4349 | 4702 | gaagUfacaUfCfccguacacasusu | 4349 | uguGfuAfcGfgGfaUfgUfaCfuUfcsusu |
| siRNA 4703/4349 | 4703 | gaagUfaCfaUfcccguacacasusu | 4349 | uguGfuAfcGfgGfaUfgUfaCfuUfcsusu |
| siRNA 4704/4349 | 4704 | gaagUfaCfaUfcCfcguacacasusu | 4349 | uguGfuAfcGfgGfaUfgUfaCfuUfcsusu |
| siRNA 4705/4349 | 4705 | gaagUfaCfaUfCfccguacacasusu | 4349 | uguGfuAfcGfgGfaUfgUfaCfuUfcsusu |
| siRNA 4316/4350 | 4316 | ucuguGfccGfGfcuacuucgasusu | 4350 | ucgAfaGfuAfgCfcGfgCfaCfaGfasusu |
| siRNA 4706/4351 | 4706 | cuacuucGfAfcugcaagucasusu | 4351 | ugaCfuUfgCfaGfuCfgAfaGfuAfgsusu |
| siRNA 4317/4351 | 4317 | cuacUfuCfGfdAcugcaagucasusu | 4351 | ugaCfuUfgCfaGfuCfgAfaGfuAfgsusu |
| siRNA 4707/4352 | 4707 | acuucgaCfUfgCfaaguccgasusu | 4352 | ucgGfaCfuUfgCfaGfuCfgAfaGfususu |
| siRNA 4708/4352 | 4708 | acuuCfgacUfgCfaaguccgasusu | 4352 | ucgGfaCfuUfgCfaGfuCfgAfaGfususu |
| siRNA 4709/4352 | 4709 | acuuCfgaCfUfgcaaguccgasusu | 4352 | ucgGfaCfuUfgCfaGfuCfgAfaGfususu |
| siRNA 4710/4352 | 4710 | acuuCfgaCfUfgCfaaguccgasusu | 4352 | ucgGfaCfuUfgCfaGfuCfgAfaGfususu |
| siRNA 4318/4353 | 4318 | uucgaCfUfgCfaaguccgacasusu | 4353 | uguCfgGfaCfuUfgCfaGfuCfgAfasusu |
| siRNA 4711/4354 | 4711 | uuaccucuAfcGfgcaucauasusu | 4354 | uauGfaUfgCfcGfuAfgAfgGfuAfasusu |
| siRNA 4319/4354 | 4319 | uuacCfuCfUfdAcGfgcaucauasusu | 4354 | uauGfaUfgCfcGfuAfgAfgGfuAfasusu |

Therapeutic siRNAs were designed to target human HGFAC as described above and, in some cases, the HGFAC sequence of at least one toxicology-relevant species, in this case, the mouse. The siRNAs included in subset H have the following characteristics and are shown in Table H:
Cross-reactivity: With 19mer in human HGFAC mRNA, with 17mer (pos. 2-18) in mouse HGFAC, allowing for one mismatch
Specificity category: For human: AS4 or better, SS4 or better. For mouse: AS4 or better, SS4 or better.
SNPs: siRNA target sites do not harbor SNPs with a MAF≥10% (pos. 2-18)

TABLE H
Subset H siRNAs

| | SEQ | Sense strand | SEQ | Antisense strand |
|---|---|---|---|---|
| siRNA | ID | sequence | ID | sequence |
| Name | NO: | (5′-3′) | NO: | (5′-3′) |
| siRNA 625 | 625 | CGGCACAGAGAAAUGCUUU | 2676 | AAAGCAUUUCUCUGUGCCG |
| siRNA 626 | 626 | GGCACAGAGAAAUGCUUUG | 2677 | CAAAGCAUUUCUCUGUGCC |
| siRNA 627 | 627 | GCACAGAGAAAUGCUUUGA | 2678 | UCAAAGCAUUUCUCUGUGC |
| siRNA 628 | 628 | CACAGAGAAAUGCUUUGAU | 2679 | AUCAAAGCAUUUCUCUGUG |
| siRNA 629 | 629 | ACAGAGAAAUGCUUUGAUG | 2680 | CAUCAAAGCAUUUCUCUGU |
| siRNA 630 | 630 | CAGAGAAAUGCUUUGAUGA | 2681 | UCAUCAAAGCAUUUCUCUG |
| siRNA 631 | 631 | AGAGAAAUGCUUUGAUGAG | 2682 | CUCAUCAAAGCAUUUCUCU |
| siRNA 634 | 634 | GAAAUGCUUUGAUGAGACC | 2685 | GGUCUCAUCAAAGCAUUUC |
| siRNA 757 | 757 | ACAUACAGCUUGUCUGAGC | 2808 | GCUCAGACAAGCUGUAUGU |
| siRNA 1533 | 1533 | UGAUCCGGCUGAAGAAGAA | 3584 | UUCUUCUUCAGCCGGAUCA |
| siRNA 1534 | 1534 | GAUCCGGCUGAAGAAGAAA | 3585 | UUUCUUCUUCAGCCGGAUC |
| siRNA 1627 | 1627 | AGGACACAAGUGCCAGAUU | 3678 | AAUCUGGCACUUGUGUCCU |
| siRNA 1938 | 1938 | CCAACUAUGUGGACUGGAU | 3989 | AUCCAGUCCACAUAGUUGG |
| siRNA 1939 | 1939 | CAACUAUGUGGACUGGAUC | 3990 | GAUCCAGUCCACAUAGUUG |
| siRNA 1942 | 1942 | CUAUGUGGACUGGAUCAAC | 3993 | GUUGAUCCAGUCCACAUAG |
| siRNA 1943 | 1943 | UAUGUGGACUGGAUCAACG | 3994 | CGUUGAUCCAGUCCACAUA |
| siRNA 1944 | 1944 | AUGUGGACUGGAUCAACGA | 3995 | UCGUUGAUCCAGUCCACAU |

In some cases, the sense strand of any of the siRNAs of subset H comprises siRNA with a particular modification pattern. In this modification pattern, position 9 counting from the 5′ end of the of the sense strand is has the 2′F modification. If position 9 of the sense strand is a pyrimidine, then all purines in the sense strand have the 2′OMe modification. If position 9 is the only pyrimidine between positions 5 and 11 of the sense stand, then position 9 is the only position with the 2′F modification in the sense strand. If position 9 and only one other base between positions 5 and 11 of the sense strand are pyrimidines, then both of these pyrimidines are the only two positions with the 2′F modification in the sense strand. If position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. If there are >2 pyrimidines between positions 5 and 11 of the sense strand, then all combinations of pyrimidines having the 2′F modification are allowed that have three to five 2′F modifications in total, provided that the sense strand does not have three 2′F modifications in a row.

If position 9 of the sense strand is a purine, then all purines in the sense strand have the 2′OMe modification. If position 9 is the only purine between positions 5 and 11 of the sense stand, then position 9 is the only position with the 2′F modification in the sense strand. If position 9 and only one other base between positions 5 and 11 of the sense strand are purines, then both of these purines are the only two positions with the 2′F modification in the sense strand. If position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. If there are >2 purines between positions 5 and 11 of the sense strand, then all combinations of purines having the 2′F modification are allowed that have three to five 2′F modifications in total, provided that the sense strand does not have three 2′F modifications in a row.

In some cases, the sense strand of any of the siRNAs of subset H comprises a modification pattern which conforms to these sense strand rules. In some cases, a 2′ deoxy substitution may be used in sense strand (see, e.g., Table H(2)).

In some cases, the antisense strand of any of the siRNAs of subset H comprises modification pattern 3AS (see, e.g., Table H(2)). The siRNAs in subset H may comprise any other modification pattern(s).

TABLE H(2)
Modified Subset G siRNAs

| | SEQ | | SEQ | |
|---|---|---|---|---|
| | ID | | ID | |
| siRNA Name | NO: | Sense strand sequence (5′-3′) | NO: | Antisense strand sequence (5′-3′) |
| siRNA 4355/4483 | 4355 | cggcacaGfAfgAfaaugcuuasusu | 4483 | usAfsaGfcAfuUfuCfuCfuGfuGfcCfgsusu |
| siRNA 4356/4483 | 4356 | cggcacaGfAfGfAfaaugcuuasusu | 4483 | usAfsaGfcAfuUfuCfuCfuGfuGfcCfgsusu |
| siRNA 4357/4483 | 4357 | cggcacAfgAfgAfaaugcuuasusu | 4483 | usAfsaGfcAfuUfuCfuCfuGfuGfcCfgsusu |
| siRNA 4358/4483 | 4358 | cggcacAfgAfGfaaaugcuuasusu | 4483 | usAfsaGfcAfuUfuCfuCfuGfuGfcCfgsusu |
| siRNA 4359/4483 | 4359 | cggcacAfGfAfGfaaaugcuuasusu | 4483 | usAfsaGfcAfuUfuCfuCfuGfuGfcCfgsusu |
| siRNA 4360/4483 | 4360 | cggcacAfGfAfGfAfaaugcuuasusu | 4483 | usAfsaGfcAfuUfuCfuCfuGfuGfcCfgsusu |
| siRNA 4361/4483 | 4361 | cggcAfcagAfgAfaaugcuuasusu | 4483 | usAfsaGfcAfuUfuCfuCfuGfuGfcCfgsusu |
| siRNA 4362/4483 | 4362 | cggcAfcagAfGfaaaugcuuasusu | 4483 | usAfsaGfcAfuUfuCfuCfuGfuGfcCfgsusu |
| siRNA 4363/4483 | 4363 | cggcAfcaGfAfgaaaugcuuasusu | 4483 | usAfsaGfcAfuUfuCfuCfuGfuGfcCfgsusu |
| siRNA 4364/4483 | 4364 | cggcAfcaGfAfgAfaaugcuuasusu | 4483 | usAfsaGfcAfuUfuCfuCfuGfuGfcCfgsusu |
| siRNA 4365/4483 | 4365 | cggcAfcaGfAfGfAfaaugcuuasusu | 4483 | usAfsaGfcAfuUfuCfuCfuGfuGfcCfgsusu |
| siRNA 4366/4483 | 4366 | cggcAfcAfgAfgaaaugcuuasusu | 4483 | usAfsaGfcAfuUfuCfuCfuGfuGfcCfgsusu |
| siRNA 4367/4483 | 4367 | cggcAfcAfgAfgAfaaugcuuasusu | 4483 | usAfsaGfcAfuUfuCfuCfuGfuGfcCfgsusu |
| siRNA 4368/4483 | 4368 | cggcAfcAfgAfGfaaaugcuuasusu | 4483 | usAfsaGfcAfuUfuCfuCfuGfuGfcCfgsusu |
| siRNA 4369/4483 | 4369 | cggcAfcAfGfAfGfaaaugcuuasusu | 4483 | usAfsaGfcAfuUfuCfuCfuGfuGfcCfgsusu |
| siRNA 4370/4484 | 4370 | ggcacagAfGfaAfaugcuuuasusu | 4484 | usAfsaAfgCfaUfuUfcUfcUfgUfgCfcsusu |
| siRNA 4371/4484 | 4371 | ggcacagAfGfAfAfaugcuuuasusu | 4484 | usAfsaAfgCfaUfuUfcUfcUfgUfgCfcsusu |
| siRNA 4372/4484 | 4372 | ggcacaGfaGfaAfaugcuuuasusu | 4484 | usAfsaAfgCfaUfuUfcUfcUfgUfgCfcsusu |
| siRNA 4373/4484 | 4373 | ggcacaGfaGfAfaaugcuuuasusu | 4484 | usAfsaAfgCfaUfuUfcUfcUfgUfgCfcsusu |
| siRNA 4374/4484 | 4374 | ggcacaGfAfGfAfaaugcuuuasusu | 4484 | usAfsaAfgCfaUfuUfcUfcUfgUfgCfcsusu |
| siRNA 4375/4484 | 4375 | ggcacaGfAfGfAfAfaugcuuuasusu | 4484 | usAfsaAfgCfaUfuUfcUfcUfgUfgCfcsusu |
| siRNA 4376/4484 | 4376 | ggcacAfgaGfaAfaugcuuuasusu | 4484 | usAfsaAfgCfaUfuUfcUfcUfgUfgCfcsusu |
| siRNA 4377/4484 | 4377 | ggcacAfgaGfAfaaugcuuuasusu | 4484 | usAfsaAfgCfaUfuUfcUfcUfgUfgCfcsusu |
| siRNA 4378/4484 | 4378 | ggcacAfgAfGfaaaugcuuuasusu | 4484 | usAfsaAfgCfaUfuUfcUfcUfgUfgCfcsusu |
| siRNA 4379/4484 | 4379 | ggcacAfgAfGfaAfaugcuuuasusu | 4484 | usAfsaAfgCfaUfuUfcUfcUfgUfgCfcsusu |
| siRNA 4380/4484 | 4380 | ggcacAfgAfGfAfAfaugcuuuasusu | 4484 | usAfsaAfgCfaUfuUfcUfcUfgUfgCfcsusu |
| siRNA 4381/4484 | 4381 | ggcacAfGfaGfaaaugcuuuasusu | 4484 | usAfsaAfgCfaUfuUfcUfcUfgUfgCfcsusu |
| siRNA 4382/4484 | 4382 | ggcacAfGfaGfaAfaugcuuuasusu | 4484 | usAfsaAfgCfaUfuUfcUfcUfgUfgCfcsusu |
| siRNA 4383/4484 | 4383 | ggcacAfGfaGfAfaaugcuuuasusu | 4484 | usAfsaAfgCfaUfuUfcUfcUfgUfgCfcsusu |
| siRNA 4384/4484 | 4384 | ggcacAfGfAfGfaaaugcuuuasusu | 4484 | usAfsaAfgCfaUfuUfcUfcUfgUfgCfcsusu |
| siRNA 4385/4484 | 4385 | ggcacAfGfAfGfaAfaugcuuuasusu | 4484 | usAfsaAfgCfaUfuUfcUfcUfgUfgCfcsusu |
| siRNA 4386/4484 | 4386 | ggcacAfGfAfGfAfaaugcuuuasusu | 4484 | usAfsaAfgCfaUfuUfcUfcUfgUfgCfcsusu |
| siRNA 4387/4485 | 4387 | gcacagaGfAfaAfugcuuugasusu | 4485 | usCfsaAfaGfcAfuUfuCfuCfuGfuGfcsusu |
| siRNA 4388/4485 | 4388 | gcacagaGfAfAfAfugcuuugasusu | 4485 | usCfsaAfaGfcAfuUfuCfuCfuGfuGfcsusu |
| siRNA 4389/4485 | 4389 | gcacagAfgAfaAfugcuuugasusu | 4485 | usCfsaAfaGfcAfuUfuCfuCfuGfuGfcsusu |
| siRNA 4390/4485 | 4390 | gcacagAfgAfAfaugcuuugasusu | 4485 | usCfsaAfaGfcAfuUfuCfuCfuGfuGfcsusu |
| siRNA 4391/4485 | 4391 | gcacagAfGfAfAfaugcuuugasusu | 4485 | usCfsaAfaGfcAfuUfuCfuCfuGfuGfcsusu |
| siRNA 4392/4485 | 4392 | gcacagAfGfAfAfAfugcuuugasusu | 4485 | usCfsaAfaGfcAfuUfuCfuCfuGfuGfcsusu |
| siRNA 4393/4485 | 4393 | gcacaGfagAfaAfugcuuugasusu | 4485 | usCfsaAfaGfcAfuUfuCfuCfuGfuGfcsusu |
| siRNA 4394/4485 | 4394 | gcacaGfagAfAfaugcuuugasusu | 4485 | usCfsaAfaGfcAfuUfuCfuCfuGfuGfcsusu |
| siRNA 4395/4485 | 4395 | gcacaGfaGfAfaaugcuuugasusu | 4485 | usCfsaAfaGfcAfuUfuCfuCfuGfuGfcsusu |
| siRNA 4396/4485 | 4396 | gcacaGfaGfAfaAfugcuuugasusu | 4485 | usCfsaAfaGfcAfuUfuCfuCfuGfuGfcsusu |
| siRNA 4397/4485 | 4397 | gcacaGfaGfAfAfAfugcuuugasusu | 4485 | usCfsaAfaGfcAfuUfuCfuCfuGfuGfcsusu |
| siRNA 4398/4485 | 4398 | gcacaGfAfgAfaaugcuuugasusu | 4485 | usCfsaAfaGfcAfuUfuCfuCfuGfuGfcsusu |
| siRNA 4399/4485 | 4399 | gcacaGfAfgAfaAfugcuuugasusu | 4485 | usCfsaAfaGfcAfuUfuCfuCfuGfuGfcsusu |
| siRNA 4400/4485 | 4400 | gcacaGfAfgAfAfaugcuuugasusu | 4485 | usCfsaAfaGfcAfuUfuCfuCfuGfuGfcsusu |
| siRNA 4401/4485 | 4401 | gcacaGfAfGfAfaaugcuuugasusu | 4485 | usCfsaAfaGfcAfuUfuCfuCfuGfuGfcsusu |
| siRNA 4402/4485 | 4402 | gcacaGfAfGfAfaAfugcuuugasusu | 4485 | usCfsaAfaGfcAfuUfuCfuCfuGfuGfcsusu |
| siRNA 4403/4485 | 4403 | gcacaGfAfGfAfAfaugcuuugasusu | 4485 | usCfsaAfaGfcAfuUfuCfuCfuGfuGfcsusu |
| siRNA 4404/4485 | 4404 | gcacAfgagAfaAfugcuuugasusu | 4485 | usCfsaAfaGfcAfuUfuCfuCfuGfuGfcsusu |
| siRNA 4405/4485 | 4405 | gcacAfgagAfAfaugcuuugasusu | 4485 | usCfsaAfaGfcAfuUfuCfuCfuGfuGfcsusu |
| siRNA 4406/4485 | 4406 | gcacAfgaGfAfaaugcuuugasusu | 4485 | usCfsaAfaGfcAfuUfuCfuCfuGfuGfcsusu |
| siRNA 4407/4485 | 4407 | gcacAfgaGfAfaAfugcuuugasusu | 4485 | usCfsaAfaGfcAfuUfuCfuCfuGfuGfcsusu |
| siRNA 4408/4485 | 4408 | gcacAfgaGfAfAfAfugcuuugasusu | 4485 | usCfsaAfaGfcAfuUfuCfuCfuGfuGfcsusu |
| siRNA 4409/4485 | 4409 | gcacAfgAfgAfaaugcuuugasusu | 4485 | usCfsaAfaGfcAfuUfuCfuCfuGfuGfcsusu |
| siRNA 4410/4485 | 4410 | gcacAfgAfgAfaAfugcuuugasusu | 4485 | usCfsaAfaGfcAfuUfuCfuCfuGfuGfcsusu |
| siRNA 4411/4485 | 4411 | gcacAfgAfgAfAfaugcuuugasusu | 4485 | usCfsaAfaGfcAfuUfuCfuCfuGfuGfcsusu |
| siRNA 4412/4485 | 4412 | gcacAfgAfGfAfAfaugcuuugasusu | 4485 | usCfsaAfaGfcAfuUfuCfuCfuGfuGfcsusu |
| siRNA 4413/4485 | 4413 | gcacAfGfagAfaaugcuuugasusu | 4485 | usCfsaAfaGfcAfuUfuCfuCfuGfuGfcsusu |
| siRNA 4414/4485 | 4414 | gcacAfGfagAfaAfugcuuugasusu | 4485 | usCfsaAfaGfcAfuUfuCfuCfuGfuGfcsusu |
| siRNA 4415/4485 | 4415 | gcacAfGfagAfAfaugcuuugasusu | 4485 | usCfsaAfaGfcAfuUfuCfuCfuGfuGfcsusu |
| siRNA 4416/4485 | 4416 | gcacAfGfaGfAfaaugcuuugasusu | 4485 | usCfsaAfaGfcAfuUfuCfuCfuGfuGfcsusu |
| siRNA 4417/4485 | 4417 | gcacAfGfaGfAfaAfugcuuugasusu | 4485 | usCfsaAfaGfcAfuUfuCfuCfuGfuGfcsusu |
| siRNA 4418/4485 | 4418 | gcacAfGfAfGfAfaaugcuuugasusu | 4485 | usCfsaAfaGfcAfuUfuCfuCfuGfuGfcsusu |
| siRNA 4419/4486 | 4419 | cacagaGfaAfAfugcuuugaasusu | 4486 | usUfscAfaAfgCfaUfuUfcUfcUfgUfgsusu |
| siRNA 4420/4486 | 4420 | cacagaGfAfAfAfugcuuugaasusu | 4486 | usUfscAfaAfgCfaUfuUfcUfcUfgUfgsusu |
| siRNA 4421/4486 | 4421 | cacagAfgaAfAfugcuuugaasusu | 4486 | usUfscAfaAfgCfaUfuUfcUfcUfgUfgsusu |
| siRNA 4422/4486 | 4422 | cacagAfgAfAfaugcuuugaasusu | 4486 | usUfscAfaAfgCfaUfuUfcUfcUfgUfgsusu |
| siRNA 4423/4486 | 4423 | cacagAfGfaAfaugcuuugaasusu | 4486 | usUfscAfaAfgCfaUfuUfcUfcUfgUfgsusu |
| siRNA 4424/4486 | 4424 | cacagAfGfaAfAfugcuuugaasusu | 4486 | usUfscAfaAfgCfaUfuUfcUfcUfgUfgsusu |
| siRNA 4425/4486 | 4425 | cacagAfGfAfAfaugcuuugaasusu | 4486 | usUfscAfaAfgCfaUfuUfcUfcUfgUfgsusu |
| siRNA 4426/4486 | 4426 | cacagAfGfAfAfAfugcuuugaasusu | 4486 | usUfscAfaAfgCfaUfuUfcUfcUfgUfgsusu |
| siRNA 4427/4486 | 4427 | cacaGfagaAfAfugcuuugaasusu | 4486 | usUfscAfaAfgCfaUfuUfcUfcUfgUfgsusu |
| siRNA 4428/4486 | 4428 | cacaGfagAfAfaugcuuugaasusu | 4486 | usUfscAfaAfgCfaUfuUfcUfcUfgUfgsusu |
| siRNA 4429/4486 | 4429 | cacaGfaGfaAfaugcuuugaasusu | 4486 | usUfscAfaAfgCfaUfuUfcUfcUfgUfgsusu |
| siRNA 4430/4486 | 4430 | cacaGfaGfaAfAfugcuuugaasusu | 4486 | usUfscAfaAfgCfaUfuUfcUfcUfgUfgsusu |
| siRNA 4431/4486 | 4431 | cacaGfaGfAfAfAfugcuuugaasusu | 4486 | usUfscAfaAfgCfaUfuUfcUfcUfgUfgsusu |
| siRNA 4432/4486 | 4432 | cacaGfAfgaAfaugcuuugaasusu | 4486 | usUfscAfaAfgCfaUfuUfcUfcUfgUfgsusu |
| siRNA 4433/4486 | 4433 | cacaGfAfgaAfAfugcuuugaasusu | 4486 | usUfscAfaAfgCfaUfuUfcUfcUfgUfgsusu |
| siRNA 4434/4486 | 4434 | cacaGfAfgAfAfaugcuuugaasusu | 4486 | usUfscAfaAfgCfaUfuUfcUfcUfgUfgsusu |
| siRNA 4435/4486 | 4435 | cacaGfAfGfAfAfaugcuuugaasusu | 4486 | usUfscAfaAfgCfaUfuUfcUfcUfgUfgsusu |
| siRNA 4436/4487 | 4436 | acagagaAfAfuGfcuuugauasusu | 4487 | usAfsuCfaAfaGfcAfuUfuCfuCfuGfususu |
| siRNA 4437/4487 | 4437 | acagagAfaAfuGfcuuugauasusu | 4487 | usAfsuCfaAfaGfcAfuUfuCfuCfuGfususu |
| siRNA 4438/4487 | 4438 | acagaGfaaAfuGfcuuugauasusu | 4487 | usAfsuCfaAfaGfcAfuUfuCfuCfuGfususu |
| siRNA 4439/4487 | 4439 | acagaGfaAfAfugcuuugauasusu | 4487 | usAfsuCfaAfaGfcAfuUfuCfuCfuGfususu |
| siRNA 4440/4487 | 4440 | acagaGfaAfAfuGfcuuugauasusu | 4487 | usAfsuCfaAfaGfcAfuUfuCfuCfuGfususu |
| siRNA 4441/4487 | 4441 | acagaGfAfaAfugcuuugauasusu | 4487 | usAfsuCfaAfaGfcAfuUfuCfuCfuGfususu |
| siRNA 4442/4487 | 4442 | acagaGfAfaAfuGfcuuugauasusu | 4487 | usAfsuCfaAfaGfcAfuUfuCfuCfuGfususu |
| siRNA 4443/4487 | 4443 | acagaGfAfAfAfugcuuugauasusu | 4487 | usAfsuCfaAfaGfcAfuUfuCfuCfuGfususu |
| siRNA 4444/4487 | 4444 | acagaGfAfAfAfuGfcuuugauasusu | 4487 | usAfsuCfaAfaGfcAfuUfuCfuCfuGfususu |
| siRNA 4445/4487 | 4445 | acagAfgaaAfuGfcuuugauasusu | 4487 | usAfsuCfaAfaGfcAfuUfuCfuCfuGfususu |
| siRNA 4446/4487 | 4446 | acagAfgaAfAfugcuuugauasusu | 4487 | usAfsuCfaAfaGfcAfuUfuCfuCfuGfususu |
| siRNA 4447/4487 | 4447 | acagAfgaAfAfuGfcuuugauasusu | 4487 | usAfsuCfaAfaGfcAfuUfuCfuCfuGfususu |
| siRNA 4448/4487 | 4448 | acagAfgAfaAfugcuuugauasusu | 4487 | usAfsuCfaAfaGfcAfuUfuCfuCfuGfususu |
| siRNA 4449/4487 | 4449 | acagAfgAfaAfuGfcuuugauasusu | 4487 | usAfsuCfaAfaGfcAfuUfuCfuCfuGfususu |
| siRNA 4450/4487 | 4450 | acagAfGfaaAfugcuuugauasusu | 4487 | usAfsuCfaAfaGfcAfuUfuCfuCfuGfususu |
| siRNA 4451/4487 | 4451 | acagAfGfaaAfuGfcuuugauasusu | 4487 | usAfsuCfaAfaGfcAfuUfuCfuCfuGfususu |
| siRNA 4452/4487 | 4452 | acagAfGfaAfAfugcuuugauasusu | 4487 | usAfsuCfaAfaGfcAfuUfuCfuCfuGfususu |
| siRNA 4453/4487 | 4453 | acagAfGfaAfAfuGfcuuugauasusu | 4487 | usAfsuCfaAfaGfcAfuUfuCfuCfuGfususu |
| siRNA 4454/4487 | 4454 | acagAfGfAfAfAfugcuuugauasusu | 4487 | usAfsuCfaAfaGfcAfuUfuCfuCfuGfususu |
| siRNA 4455/4488 | 4455 | cagagaaaUfgCfuuugaugasusu | 4488 | usCfsaUfcAfaAfgCfaUfuUfcUfcUfgsusu |
| siRNA 4456/4488 | 4456 | cagaGfaAfAfdTgCfuuugaugasusu | 4488 | usCfsaUfcAfaAfgCfaUfuUfcUfcUfgsusu |
| siRNA 4457/4489 | 4457 | agagaAfAfuGfcuuugaugaasusu | 4489 | usUfscAfuCfaAfaGfcAfuUfuCfuCfususu |
| siRNA 4458/4489 | 4458 | agagAfaAfuGfcuuugaugaasusu | 4489 | usUfscAfuCfaAfaGfcAfuUfuCfuCfususu |
| siRNA 4459/4489 | 4459 | agagAfAfauGfcuuugaugaasusu | 4489 | usUfscAfuCfaAfaGfcAfuUfuCfuCfususu |
| siRNA 4460/4490 | 4460 | gaaaugCfuUfUfgaugagacasusu | 4490 | usGfsuCfuCfaUfcAfaAfgCfaUfuUfcsusu |
| siRNA 4461/4490 | 4461 | gaaaugCfUfUfUfgaugagacasusu | 4490 | usGfsuCfuCfaUfcAfaAfgCfaUfuUfcsusu |
| siRNA 4462/4490 | 4462 | gaaaUfgcuUfUfgaugagacasusu | 4490 | usGfsuCfuCfaUfcAfaAfgCfaUfuUfcsusu |
| siRNA 4463/4490 | 4463 | gaaaUfgcUfUfugaugagacasusu | 4490 | usGfsuCfuCfaUfcAfaAfgCfaUfuUfcsusu |
| siRNA 4464/4490 | 4464 | gaaaUfgCfuUfugaugagacasusu | 4490 | usGfsuCfuCfaUfcAfaAfgCfaUfuUfcsusu |
| siRNA 4465/4490 | 4465 | gaaaUfgCfuUfUfgaugagacasusu | 4490 | usGfsuCfuCfaUfcAfaAfgCfaUfuUfcsusu |
| siRNA 4466/4490 | 4466 | gaaaUfgCfUfUfUfgaugagacasusu | 4490 | usGfsuCfuCfaUfcAfaAfgCfaUfuUfcsusu |
| siRNA 4467/4491 | 4467 | acauaCfagCfuUfgucugagasusu | 4491 | usCfsuCfaGfaCfaAfgCfuGfuAfuGfususu |
| siRNA 4468/4491 | 4468 | acauaCfagCfUfugucugagasusu | 4491 | usCfsuCfaGfaCfaAfgCfuGfuAfuGfususu |
| siRNA 4469/4492 | 4469 | ugaucCfggCfUfgaagaagaasusu | 4492 | usUfscUfuCfuUfcAfgCfcGfgAfuCfasusu |
| siRNA 4470/4492 | 4470 | ugauCfcggCfUfgaagaagaasusu | 4492 | usUfscUfuCfuUfcAfgCfcGfgAfuCfasusu |
| siRNA 4471/4492 | 4471 | ugauCfCfggCfugaagaagaasusu | 4492 | usUfscUfuCfuUfcAfgCfcGfgAfuCfasusu |
| siRNA 4472/4492 | 4472 | ugauCfCfggCfUfgaagaagaasusu | 4492 | usUfscUfuCfuUfcAfgCfcGfgAfuCfasusu |
| siRNA 4473/4493 | 4473 | gaucCfggCfUfgaagaagaaasusu | 4493 | usUfsuCfuUfcUfuCfaGfcCfgGfaUfcsusu |
| siRNA 4474/4494 | 4474 | aggacAfcaAfGfugccagauasusu | 4494 | usAfsuCfuGfgCfaCfuUfgUfgUfcCfususu |
| siRNA 4475/4494 | 4475 | aggacAfcAfAfgugccagauasusu | 4494 | usAfsuCfuGfgCfaCfuUfgUfgUfcCfususu |
| siRNA 4476/4495 | 4476 | ccaacuAfuGfuGfgacuggaasusu | 4495 | usUfscCfaGfuCfcAfcAfuAfgUfuGfgsusu |
| siRNA 4477/4496 | 4477 | caacUfaUfgUfggacuggauasusu | 4496 | usAfsuCfcAfgUfcCfaCfaUfaGfuUfgsusu |
| siRNA 4478/4497 | 4478 | cuauGfugGfAfcuggaucaaasusu | 4497 | usUfsuGfaUfcCfaGfuCfcAfcAfuAfgsusu |
| siRNA 4479/4497 | 4479 | cuauGfuGfgAfcuggaucaaasusu | 4497 | usUfsuGfaUfcCfaGfuCfcAfcAfuAfgsusu |
| siRNA 4480/4498 | 4480 | uaugUfggaCfUfggaucaacasusu | 4498 | usGfsuUfgAfuCfcAfgUfcCfaCfaUfasusu |
| siRNA 4481/4499 | 4481 | auguggaCfUfggaucaacgasusu | 4499 | usCfsgUfuGfaUfcCfaGfuCfcAfcAfususu |
| siRNA 4482/4499 | 4482 | auguGfgAfCfdTggaucaacgasusu | 4499 | usCfsgUfuGfaUfcCfaGfuCfcAfcAfususu |

### Example 3: siRNA-Mediated Knockdown of HGFAC in HepG2 Cell Line

siRNAs targeted to HGFAC mRNA that downregulate levels of HGFAC mRNA are expected to lead to a decrease in HGFAC activation when administered to the cultured human hepatocyte cell line, HepG2.

On Day 0, the HepG2 cells are seeded at 150,000 cells/mL into a Falcon 24-well tissue culture plate (ThermoFisher Cat. No. 353047) at 0.5 mL per well.

On Day 1, the HGFAC siRNA and negative control siRNA master mixes are prepared. The HGFAC siRNA master mix contains 350 μL of Opti-MEM (ThermoFisher Cat. No. 4427037-s1288 Lot No. AS02B02D) and 3.5 μL of a mixture of two HGFAC siRNAs (10 μM stock). The negative control siRNA master mix contains 350 μL of Opti-MEM and 3.5 μL of negative control siRNA (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HepG2 cells with a final siRNA concentration of 10 nM.

On Day 4, 72 hours post transfection, the cells are lysed using the Cells-to-Ct kit according to the manufacturer's protocol (ThermoFisher Cat. No. 4399002). For the Cells-to-Ct protocol, cells are washed with 50 μL using cold 1× PBS and lysed by adding 49.5 μL of Lysis Solution and 0.5 μL DNase I per well and pipetting up and down 5 times and incubating for 5 minutes at room temperature. The Stop Solution (5 μL/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/HGFAC using a BioRad CFX96 Cat. No. 1855195). For the protein quantification, equivalent quantities (30-50 g) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. The primary immunoblotting antibody used is anti-HGFAC (R&D Systems Cat. No. AF1514).

A decrease in HGFAC mRNA expression in the HepG2 cells is expected after transfection with the HGFAC siRNAs compared to HGFAC mRNA levels in HepG2 cells transfected with the non-specific control siRNA 72 hours after transfection. There is also an expected decrease in the amount of activated HGFAC, measured by quantifying the total amount of pro-HGFAC relative to enzymatically active HGFAC in culture media of HepG2 cells transfected with the HGFAC siRNAs compared to the amount of pro-HGFAC relative to enzymatically active HGFAC in culture media of HepG2 cells 72 hours after transfection. These results show that the HGFAC siRNAs elicit knockdown of HGFAC mRNA in HepG2 cells and that the decrease in HGFAC expression is correlated with a decrease both pro-HGFAC and activated HGFAC.

### Example 4: ASO-Mediated Knockdown of HGFAC in HepG2 Cell Line

ASOs targeted to HGFAC mRNA that downregulate levels of HGFAC mRNA are expected to lead to a decrease in HGFAC activation when administered to the cultured human hepatocyte cell line, HepG2.

On Day 0, the HepG2 cells are seeded at 150,000 cells/mL into a Falcon 24-well tissue culture plate (ThermoFisher Cat. No. 353047) at 0.5 mL per well.

On Day 1, the HGFAC ASO and negative control ASO master mixes are prepared. The HGFAC ASO master mix contains 350 μL of Opti-MEM (ThermoFisher Cat. No. 4427037-s1288 Lot No. AS02B02D) and 3.5 μL of a mixture of two HGFAC ASOs (10 μM stock). The negative control ASO master mix contains 350 μL of Opti-MEM and 3.5 μL of negative control ASO (ThermoFisher Cat. No. 4390843, 10 μM stock). Next, 3 μL of TransIT-X2 (Mirus Cat. No. MIR-6000) is added to each master mix. The mixes are incubated for 15 minutes to allow transfection complexes to form, then 51 μL of the appropriate master mix+TransIT-X2 is added to duplicate wells of HepG2 cells with a final ASO concentration of 10 nM.

On Day 4, 72 hours post transfection, the cells are lysed using the Cells-to-Ct kit according to the manufacturer's protocol (ThermoFisher Cat. No. 4399002). For the Cells-to-Ct, cells are washed with 50 μL using cold 1× PBS and lysed by adding 49.5 μL of Lysis Solution and 0.5 μL DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 μL of the lysate according to the manufacturer's protocol. Samples are stored at −80° C. until real-time qPCR is performed in triplicate using TaqMan Gene Expression Assays (Applied Biosystems FAM/HGFAC using a BioRad CFX96 Cat. No. 1855195). For the protein quantification, equivalent quantities (30-50 g) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. The primary immunoblotting antibody used is anti-HGFAC (R&D Systems Cat. No. AF1514).

A decrease in HGFAC mRNA expression in the HepG2 cells is expected after transfection with the HGFAC ASOs compared to HGFAC mRNA levels in HepG2 cells transfected with the non-specific control ASO 72 hours after transfection. There is also an expected decrease in the amount of activated HGFAC, measured by quantifying the total amount of pro-HGFAC relative to enzymatically active HGFAC in culture media of HepG2 cells transfected with the HGFAC ASOs compared to the amount of pro-HGFAC relative to enzymatically active HGFAC in culture media of HepG2 cells 72 hours after transfection. These results show that the HGFAC ASOs elicit knockdown of HGFAC mRNA in HepG2 cells and that the decrease in HGFAC expression is correlated with a decrease both pro-HGFAC and activated HGFAC Example 5: Inhibition of HGFAC in a mouse model of breast cancer using HGFAC siRNAs or ASOs

In this experiment, a mouse model of breast cancer is used to evaluate the effect of siRNA or ASO inhibition of HGFAC. It is hypothesized that HGFAC inhibition alone or cooperative inhibition with the approved checkpoint inhibitor anti-CTLA-4 (aCTLA-4) will result in improved anti-tumor responses relative to aCTLA-4 alone. The well-characterized MMTV-PyMT model is used to investigate this hypothesis.

To be able to track antigen-specific CD8+ T-cell responses, tumor cells are engineered to express a model antigen: specifically, a fragment of Lymphocytic Choriomeningitis Virus (LCMV) nucleoprotein that produces an immunodominant MHC-I associated peptide, NP118 (RPQASGVYM) in FVB hosts (hereafter referred to as PyMT-NP tumor cells). To investigate the immune landscape of mice in each treatment group, the frequency of CD8+ T cells will be quantified in secondary lymphoid organs and in tumor-infiltrating lymphocytes.

PyMT-NP tumor cells are orthotopically transplanted into the mammary fat pad; specifically, 20,000 PyMT-NP cells are transplanted unilaterally into the fourth inguinal mammary fat pads of 4-6 week old mice. When tumors reach 100 mm³, mice will be randomized into eight experimental groups: Group 1—a group treated with non-targeting control siRNA, Group 2—a group treated with non-targeting control ASO, Group 3—a group treated with HGFAC siRNA, Group 4—a group treated with HGFAC ASO, Group 5—a group treated with HGFAC siRNA and aCTLA-4, Group 6—a group treated with HGFAC ASO and aCTLA-4 Group 7—a group treated with aCTLA-4, Group 8—a group treated with vehicle only. Each group contains eight female mice.

Administration of siRNA or ASO is achieved with a single (day 0) 200 ul subcutaneous injection of siRNA or ASO resuspended in PBS at a concentration of 10 uM. Administration of aCTLA-4 is achieved with a twice weekly (beginning at day 0) intraperitoneal injection of antibody resuspended in DMSO at a concentration of 10 mg/kg. Twice weekly antibody injections are carried out for three weeks, and mice are sacrificed on day 24. Response to therapy is assessed using three metrics: by quantifying the CD8+ T cell response, by assessing the tumor growth rate and by determining the number and proportion of mice experiencing clinical benefit (defined as both complete or partial response to treatment). Similar to clinical response of humans to immunotherapeutics, it is expected that some subjects (mice) will not respond to immunotherapy at all, while others will experience slower tumor growth and some will experience eradication of the tumor altogether.

To assess siRNA and ASO mediated gene knockdown, mRNA is isolated from hepatic tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/HGFAC using a BioRad CFX96 Cat. No. 1855195). A decrease in HGFAC mRNA expression in the liver tissue and circulating HGFAC protein in the blood from mice dosed with the HGFAC siRNA or ASO is expected compared to HGFAC mRNA expression in the liver tissue and circulating HGFAC protein in the blood from mice dosed with the non-specific controls.

### Example 6. Testing the Activity of HGFAC siRNAs ETD02131-ETD02253 in Mice Transfected with AAV8-TBG-h-HGFAC

The activities of siRNAs, namely ETD02131-ETD02253, were assessed. The siRNAs were attached to the GalNAc ligand ETL17 followed by a phosphorothioate linkage at the 5′ end of the sense strand. The siRNAs used in this Example are included in Table 4, where Nf is a 2′ fluoro-modified nucleoside, n is a 2′ O-methyl modified nucleoside, “d” is a 2′ deoxynucleoside, and “s” is a phosphorothioate linkage. The base sequences for each siRNA, with and without the 3′ UU extension, are shown in Table 5, ETD02131-ETD02141 were tested in part 1 of the study and ETD02242-ETD02253 were tested in part 2.

Six to eight week old female mice (C57Bl/6) were injected with 5 μL of a recombinant adeno-associated virus 8 (AAV8) vector (2.0×10E13 genome copies/mL, study part 1 or 1.8×10E13 genome copies/mL, study part 2) by the retroorbital or tail vein route. The recombinant AAV8 contained the open reading frame and the majority of the 3′UTR of the human HGFAC sequence (GenBank Accession #BC 112190) under the control of the human thyroxine binding globulin promoter in an AAV2 backbone packaged in AAV8 capsid (AAV8-TBG-h-HGFAC).

On Day 17 (study part 1) or on Day 20 (study part 2) after infection mice were given a subcutaneous injection of a single 100 μg dose of a GalNAc-conjugated siRNA or PBS as vehicle control. On Day 0 and on Days 5 and 11 after subcutaneous injection, serum was collected to assess levels of human HGFAC. The serum level of human HGFAC in each mouse was measured using the DuoSet Human HGF Activator ELISA kit (R&D Systems, Catalog #DY1514) according to the Manufacturer's instructions. The plate was analyzed on an Envision 2105 Multimode Plate Reader (PerkinElmer). The concentration of HGFAC in each mouse serum sample was calculated from the standard curve by interpolation using least squares fit (Prism version 9, Software MacKiev). The human HGFAC serum concentration at each timepoint was made relative to the level of HGFAC of each individual mouse on Day 0. Outliers were identified using Grubbs' Test. The results of part 1 of the study are shown in Table 6, and the results from part 2 are shown in Table 7.

Mice were euthanized on Day 11 and a liver sample from each was collected and placed in RNAlater (ThermoFisher Cat #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver HGFAC mRNA were assessed by RT-qPCR in triplicate on a QuantStudio™ 6 Pro Real-Time PCR System using TaqMan assays for human HGFAC (ThermoFisher, assay #Hs00173526_ml) and the mouse housekeeping gene PPIA (ThermoFisher, assay #Mm02342430_g1) and PerfeCTa® qPCR FastMix®, Low ROX™ (VWR, Catalog #101419-222). Mice with undetectable hHGFAC expression were omitted from further analysis. Data were normalized to the level in animals receiving PBS. The results of part 1 of the study are shown in Table 8, and the results from part 2 are shown in Table 8.

TABLE 4
Example siRNA Sequences

| siRNA | SEQ ID | Sense Strand Sequence (5′-3′) | SEQ ID | Antisense Strand Sequence |
|---|---|---|---|---|
| Name | NO: | with GalNAc moiety | NO: | (5′-3′) |
| ETD02231 | 4500 | [ETL17]saaccuAfAfuGfccacag | 4320 | usUfscGfcUfgUfgGfcAfuUfa |
| | | cgaasusu | | GfgUfususu |
| ETD02232 | 4501 | [ETL17]sacuaUfcCfUfdGGfug | 4211 | usAfsgAfgGfuCfaCfcAfgGfa |
| | | accucuasusu | | UfaGfususu |
| ETD02233 | 4502 | [ETL17]suggugUfgCfCfaCfaac | 4321 | usGfsuGfaGfuUfgUfgGfcAfc |
| | | ucacasusu | | AfcCfasusu |
| ETD02234 | 4503 | [ETL17]sugugccAfcAfAfcucac | 4212 | usGfsuUfgUfgAfgUfuGfuGfg |
| | | aacasusu | | CfaCfasusu |
| ETD02235 | 4504 | [ETL17]sacaaCfUfcaCfaacuacg | 4215 | usGfsuCfgUfaGfuUfgUfgAfg |
| | | acasusu | | UfuGfususu |
| ETD02236 | 4505 | [ETL17]saacucAfcAfAfcuacga | 4322 | usCfsgGfuCfgUfaGfuUfgUfg |
| | | ccgasusu | | AfgUfususu |
| ETD02237 | 4506 | [ETL17]sucacAfAfcuAfcGfacc | 4323 | usUfscCfcGfgUfcGfuAfgUfu |
| | | gggaasusu | | GfuGfasusu |
| ETD02238 | 4507 | [ETL17]sagaaGfAfGfGfAfcgu | 4324 | usGfscAfgGfaAfcGfuCfcUfc |
| | | uccugcasusu | | UfuCfususu |
| ETD02239 | 4508 | [ETL17]sagcaCfUfUfCfUfucaa | 4325 | usUfsgCfgGfuUfgAfaGfaAfg |
| | | ccgcaasusu | | UfgCfususu |
| ETD02240 | 4509 | [ETL17]scacuUfCfUfUfCfaacc | 4326 | usCfsgUfgCfgGfuUfgAfaGfa |
| | | gcacgasusu | | AfgUfgsusu |
| ETD02241 | 4510 | [ETL17]sacuuCfuUfCfdAAfcc | 4220 | usUfscGfuGfcGfgUfuGfaAfg |
| | | gcacgaasusu | | AfaGfususu |
| ETD02242 | 4511 | [ETL17]scuucUfuCfAfdAccgc | 4327 | usGfsuCfgUfgCfgGfuUfgAfa |
| | | acgacasusu | | GfaAfgsusu |
| ETD02243 | 4512 | [ETL17]suucuUfCfaaCfCfgcac | 4328 | usCfsgUfcGfuGfcGfgUfuGfa |
| | | gacgasusu | | AfgAfasusu |
| ETD02244 | 4513 | [ETL17]sucuuCfaaCfCfgCfacg | 4329 | usCfscGfuCfgUfgCfgGfuUfg |
| | | acggasusu | | AfaGfasusu |
| ETD02245 | 4514 | [ETL17]suucggCfaUfCfgagaag | 4222 | usGfsuAfcUfuCfuCfgAfuGfc |
| | | uacasusu | | CfgAfasusu |
| ETD02246 | 4515 | [ETL17]saucgAfGfAfAfGfuac | 4224 | usCfsgGfgAfuGfuAfcUfuCfu |
| | | aucccgasusu | | CfgAfususu |
| ETD02247 | 4516 | [ETL17]sucgaGfaAfGfdTaCfa | 4225 | usAfscGfgGfaUfgUfaCfuUfc |
| | | ucccguasusu | | UfcGfasusu |
| ETD02248 | 4517 | [ETL17]sgaagUfaCfaUfCfccgu | 4227 | usGfsuGfuAfcGfgGfaUfgUfa |
| | | acacasusu | | CfuUfcsusu |
| ETD02249 | 4518 | [ETL17]sucuguGfccGfGfcuacu | 4330 | usCfsgAfaGfuAfgCfcGfgCfa |
| | | ucgasusu | | CfaGfasusu |
| ETD02250 | 4519 | [ETL17]scuacUfuCfGfdAcugc | 4231 | usGfsaCfuUfgCfaGfuCfgAfa |
| | | aagucasusu | | GfuAfgsusu |
| ETD02251 | 4520 | [ETL17]sacuuCfgaCfUfgCfaag | 4232 | usCfsgGfaCfuUfgCfaGfuCfg |
| | | uccgasusu | | AfaGfususu |
| ETD02252 | 4521 | [ETL17]suucgaCfUfgCfaagucc | 4331 | usGfsuCfgGfaCfuUfgCfaGfu |
| | | gacasusu | | CfgAfasusu |
| ETD02253 | 4522 | [ETL17]suuacCfuCfUfdAcGfg | 4233 | usAfsuGfaUfgCfcGfuAfgAfg |
| | | caucauasusu | | GfuAfasusu |

TABLE 5
Example siRNA BASE Sequences

| | SEQ | Sense Strand | SEQ | Antisense Strand |
|---|---|---|---|---|
| siRNA | ID | Base Sequence | ID | Base Sequence |
| Name | NO: | (5′ to 3′) | NO: | (5′ to 3′) |
| ETD02231 | 4562 | AACCUAAUGCCACAGCGAAUU | 4617 | UUCGCUGUGGCAUUAGGUUUU |
| ETD02232 | 4563 | ACUAUCCUGGUGACCUCUAUU | 4618 | UAGAGGUCACCAGGAUAGUUU |
| ETD02233 | 4564 | UGGUGUGCCACAACUCACAUU | 4619 | UGUGAGUUGUGGCACACCAUU |
| ETD02234 | 4565 | UGUGCCACAACUCACAACAUU | 4620 | UGUUGUGAGUUGUGGCACAUU |
| ETD02235 | 4566 | ACAACUCACAACUACGACAUU | 4621 | UGUCGUAGUUGUGAGUUGUUU |
| ETD02236 | 4567 | AACUCACAACUACGACCGAUU | 4622 | UCGGUCGUAGUUGUGAGUUUU |
| ETD02237 | 4568 | UCACAACUACGACCGGGAAUU | 4623 | UUCCCGGUCGUAGUUGUGAUU |
| ETD02238 | 4569 | AGAAGAGGACGUUCCUGCAUU | 4624 | UGCAGGAACGUCCUCUUCUUU |
| ETD02239 | 4570 | AGCACUUCUUCAACCGCAAUU | 4625 | UUGCGGUUGAAGAAGUGCUUU |
| ETD02240 | 4571 | CACUUCUUCAACCGCACGAUU | 4626 | UCGUGCGGUUGAAGAAGUGUU |
| ETD02241 | 4572 | ACUUCUUCAACCGCACGAAUU | 4627 | UUCGUGCGGUUGAAGAAGUUU |
| ETD02242 | 4573 | CUUCUUCAACCGCACGACAUU | 4628 | UGUCGUGCGGUUGAAGAAGUU |
| ETD02243 | 4574 | UUCUUCAACCGCACGACGAUU | 4629 | UCGUCGUGCGGUUGAAGAAUU |
| ETD02244 | 4575 | UCUUCAACCGCACGACGGAUU | 4630 | UCCGUCGUGCGGUUGAAGAUU |
| ETD02245 | 4576 | UUCGGCAUCGAGAAGUACAUU | 4631 | UGUACUUCUCGAUGCCGAAUU |
| ETD02246 | 4577 | AUCGAGAAGUACAUCCCGAUU | 4632 | UCGGGAUGUACUUCUCGAUUU |
| ETD02247 | 4578 | UCGAGAAGTACAUCCCGUAUU | 4633 | UACGGGAUGUACUUCUCGAUU |
| ETD02248 | 4579 | GAAGUACAUCCCGUACACAUU | 4634 | UGUGUACGGGAUGUACUUCUU |
| ETD02249 | 4580 | UCUGUGCCGGCUACUUCGAUU | 4635 | UCGAAGUAGCCGGCACAGAUU |
| ETD02250 | 4581 | CUACUUCGACUGCAAGUCAUU | 4636 | UGACUUGCAGUCGAAGUAGUU |
| ETD02251 | 4582 | ACUUCGACUGCAAGUCCGAUU | 4637 | UCGGACUUGCAGUCGAAGUUU |
| ETD02252 | 4583 | UUCGACUGCAAGUCCGACAUU | 4638 | UGUCGGACUUGCAGUCGAAUU |
| ETD02253 | 4584 | UUACCUCUACGGCAUCAUAUU | 4639 | UAUGAUGCCGUAGAGGUAAUU |
| | | Sense Strand | | Antisense Strand |
| | SEQ | Base Sequence | SEQ | Base Sequence |
| siRNA | ID | (5′ to 3′), | ID | (5′ to 3′), |
| Name | NO: | without 3′ overhangs | NO: | without 3′ overhangs |
| ETD02231 | 4757 | AACCUAAUGCCACAGCGAA | 4780 | UUCGCUGUGGCAUUAGGUU |
| ETD02232 | 4758 | ACUAUCCUGGUGACCUCUA | 4781 | UAGAGGUCACCAGGAUAGU |
| ETD02233 | 4759 | UGGUGUGCCACAACUCACA | 4782 | UGUGAGUUGUGGCACACCA |
| ETD02234 | 4760 | UGUGCCACAACUCACAACA | 4783 | UGUUGUGAGUUGUGGCACA |
| ETD02235 | 4761 | ACAACUCACAACUACGACA | 4784 | UGUCGUAGUUGUGAGUUGU |
| ETD02236 | 4762 | AACUCACAACUACGACCGA | 4785 | UCGGUCGUAGUUGUGAGUU |
| ETD02237 | 4763 | UCACAACUACGACCGGGAA | 4786 | UUCCCGGUCGUAGUUGUGA |
| ETD02238 | 4764 | AGAAGAGGACGUUCCUGCA | 4787 | UGCAGGAACGUCCUCUUCU |
| ETD02239 | 4765 | AGCACUUCUUCAACCGCAA | 4788 | UUGCGGUUGAAGAAGUGCU |
| ETD02240 | 4766 | CACUUCUUCAACCGCACGA | 4789 | UCGUGCGGUUGAAGAAGUG |
| ETD02241 | 4767 | ACUUCUUCAACCGCACGAA | 4790 | UUCGUGCGGUUGAAGAAGU |
| ETD02242 | 4768 | CUUCUUCAACCGCACGACA | 4791 | UGUCGUGCGGUUGAAGAAG |
| ETD02243 | 4769 | UUCUUCAACCGCACGACGA | 4792 | UCGUCGUGCGGUUGAAGAA |
| ETD02244 | 4770 | UCUUCAACCGCACGACGGA | 4793 | UCCGUCGUGCGGUUGAAGA |
| ETD02245 | 4771 | UUCGGCAUCGAGAAGUACA | 4794 | UGUACUUCUCGAUGCCGAA |
| ETD02246 | 4772 | AUCGAGAAGUACAUCCCGA | 4795 | UCGGGAUGUACUUCUCGAU |
| ETD02247 | 4773 | UCGAGAAGTACAUCCCGUA | 4796 | UACGGGAUGUACUUCUCGA |
| ETD02248 | 4774 | GAAGUACAUCCCGUACACA | 4797 | UGUGUACGGGAUGUACUUC |
| ETD02249 | 4775 | UCUGUGCCGGCUACUUCGA | 4798 | UCGAAGUAGCCGGCACAGA |
| ETD02250 | 4776 | CUACUUCGACUGCAAGUCA | 4799 | UGACUUGCAGUCGAAGUAG |
| ETD02251 | 4777 | ACUUCGACUGCAAGUCCGA | 4800 | UCGGACUUGCAGUCGAAGU |
| ETD02252 | 4778 | UUCGACUGCAAGUCCGACA | 4801 | UGUCGGACUUGCAGUCGAA |
| ETD02253 | 4779 | UUACCUCUACGGCAUCAUA | 4802 | UAUGAUGCCGUAGAGGUAA |

TABLE 6
Relative Mean Serum Human HGFAC Levels
in AAV8-TBG-h-HGFAC Mice, Study Part 1

| | | | | Mean Serum Human HGFAC | | |
|---|---|---|---|---|---|---|
| | | | Dose | (Relative to Day 0) | | |
| Group | n | Treatment | (ug) | Day 0 | Day 5 | Day 11 |
| 1 | 4 | PBS | | 1.00 | 0.46 | 1.38 |
| 2 | 3 | ETD02231 | 100 | 1.00 | 0.47 | 0.71 |
| 3 | 4 | ETD02232 | 100 | 1.00 | 0.37 | 1.17 |
| 4 | 4 | ETD02233 | 100 | 1.00 | 0.18 | 0.31 |
| 5 | 3 | ETD02234 | 100 | 1.00 | 0.22 | 0.89 |
| 6 | 4 | ETD02235 | 100 | 1.00 | 0.47 | 0.97 |
| 7 | 4 | ETD02236 | 100 | 1.00 | 0.38 | 0.65 |
| 8 | 4 | ETD02237 | 100 | 1.00 | 0.66 | 1.03 |
| 9 | 4 | ETD02238 | 100 | 1.00 | 0.70 | 0.32 |
| 10 | 3 | ETD02239 | 100 | 1.00 | 0.19 | 0.11 |
| 11 | 4 | ETD02240 | 100 | 1.00 | 1.18 | 0.83 |
| 12 | 3 | ETD02241 | 100 | 1.00 | 0.56 | 0.54 |

TABLE 7
Relative Mean Serum Human HGFAC Levels
in AAV8-TBG-h-HGFAC Mice, Study Part 2

| | | | | Mean Serum Human HGFAC | | |
|---|---|---|---|---|---|---|
| | | | Dose | (Relative to Day 0) | | |
| Group | n | Treatment | (ug) | Day 0 | Day 5 | Day 11 |
| 1 | 3 | PBS | | 1.00 | 0.54 | 0.86 |
| 2 | 4 | ETD02242 | 100 | 1.00 | 0.72 | 0.37 |
| 3 | 4 | ETD02243 | 100 | 1.00 | 1.35 | 1.04 |
| 4 | 4 | ETD02244 | 100 | 1.00 | 0.74 | 0.98 |
| 5 | 2 | ETD02245 | 100 | 1.00 | 0.57 | 0.78 |
| 6 | 4 | ETD02246 | 100 | 1.00 | 0.32 | 0.33 |
| 7 | 3 | ETD02247 | 100 | 1.00 | 0.76 | 0.18 |
| 8 | 3 | ETD02248 | 100 | 1.00 | 0.33 | 1.11 |
| 9 | 3 | ETD02249 | 100 | 1.00 | 0.37 | 0.59 |
| 10 | 1 | ETD02250 | 100 | 1.00 | 0.61 | 0.23 |
| 11 | 3 | ETD02251 | 100 | 1.00 | 0.76 | 0.34 |
| 12 | 4 | ETD02252 | 100 | 1.00 | 1.24 | 0.72 |
| 13 | 3 | ETD02253 | 100 | 1.00 | 0.17 | 0.19 |

TABLE 8
Relative Human HGFAC mRNA Levels in Livers
of AAV8-TBG-h-HGFAC Mice, Study Part 1

| | | | Dose | Mean Human HGFAC mRNA |
|---|---|---|---|---|
| Group | n | Treatment | (ug) | (Relative to Group 1, Day 11) |
| 1 | 4 | PBS | | 1.00 |
| 2 | 4 | ETD02231 | 100 | 0.19 |
| 3 | 4 | ETD02232 | 100 | 0.51 |
| 4 | 4 | ETD02233 | 100 | 0.26 |
| 5 | 4 | ETD02234 | 100 | 1.60 |
| 6 | 4 | ETD02235 | 100 | 0.85 |
| 7 | 3 | ETD02236 | 100 | 0.36 |
| 8 | 3 | ETD02237 | 100 | 0.92 |
| 9 | 4 | ETD02238 | 100 | 0.58 |
| 10 | 4 | ETD02239 | 100 | 0.32 |
| 11 | 3 | ETD02240 | 100 | 0.37 |
| 12 | 4 | ETD02241 | 100 | 0.36 |

TABLE 9
Relative Human HGFAC mRNA Levels in Livers
of AAV8-TBG-h-HGFAC Mice, Study Part 2

| | | | Dose | Mean Human HGFAC mRNA |
|---|---|---|---|---|
| Group | n | Treatment | (ug) | (Relative to Group 1, Day 11) |
| 1 | 4 | PBS | | 1.00 |
| 2 | 4 | ETD02242 | 100 | 0.90 |
| 3 | 4 | ETD02243 | 100 | 0.84 |
| 4 | 4 | ETD02244 | 100 | 0.99 |
| 5 | 3 | ETD02245 | 100 | 2.19 |
| 6 | 4 | ETD02246 | 100 | 0.70 |
| 7 | 3 | ETD02247 | 100 | 0.29 |
| 8 | 4 | ETD02248 | 100 | 0.74 |
| 9 | 3 | ETD02249 | 100 | 0.30 |
| 10 | 3 | ETD02250 | 100 | 0.09 |
| 11 | 3 | ETD02251 | 100 | 0.36 |
| 12 | 4 | ETD02252 | 100 | 0.28 |
| 13 | 4 | ETD02253 | 100 | 0.13 |

### Example 7. Testing the Activity of HGFAC siRNAs ETD02081-ETD02103 in Mice Transfected with AAV8-TBG-h-HGFAC

The activities of siRNAs, namely ETD02081-ETD02103, were assessed. These siRNAs have the identical sequence and modification pattern as ETD02131-ETD02253, but with a 2′OMe at position 2 of the AS strand. The siRNAs were attached to the GalNAc ligand ETL17 followed by a phosphorothioate linkage at the 5′ end of the sense strand. The siRNAs used in this Example are included in Table 10, where Nf is a 2′ fluoro-modified nucleoside, n is a 2′ O-methyl modified nucleoside, “d” is a 2′ deoxynucleoside, and “s” is a phosphorothioate linkage. The base sequences are the same as those in Table 5. ETD02081-ETD02092 were tested in part 1 of the study and ETD02092-ETD02103 were tested in part 2.

Six to eight week old female mice (C57Bl/6) were injected with 5 μL of a recombinant adeno-associated virus 8 (AAV8) vector (2.0×10E13 genome copies/mL) by the retroorbital or tail vein route. The recombinant AAV8 contained the open reading frame and the majority of the 3′UTR of the human HGFAC sequence (GenBank Accession #BC 112190) under the control of the human thyroxine binding globulin promoter in an AAV2 backbone packaged in AAV8 capsid (AAV8-TBG-h-HGFAC).

On Day 17 (study part 1) or on Day 16 (study part 2) after infection mice were given a subcutaneous injection of a single 100 μg dose of a GalNAc-conjugated siRNA or PBS as vehicle control. On Day 11 after subcutaneous injection, mice were euthanized and a liver sample from each was collected and placed in RNAlater (ThermoFisher Cat #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver HGFAC mRNA were assessed by RT-qPCR in triplicate on a QuantStudio™ 6 Pro Real-Time PCR System using TaqMan assays for human HGFAC (ThermoFisher, assay #Hs00173526_ml) and the mouse housekeeping gene PPIA (ThermoFisher, assay #Mm02342430_g1) and PerfeCTa® qPCR FastMix®, Low ROX™ (VWR, Catalog #101419-222). Mice with undetectable hHGFAC expression were omitted from further analysis. Data were normalized to the level in animals receiving PBS. The results of part 1 of the study are shown in Table 11, and the results from part 2 are shown in Table 12.

TABLE 10
Example siRNA Sequences

| | SEQ | Sense Strand | SEQ | Antisense Strand |
|---|---|---|---|---|
| siRNA | ID | Sequence (5′-3′) | ID | Sequence |
| Name | NO: | with GalNAc moiety | NO: | (5′-3′) |
| ETD02081 | 4523 | [ETL17]saaccuAfAfuGfccacag | 4332 | uucGfcUfgUfgGfcAfuUfaGfg |
| | | cgaasusu | | Ufususu |
| ETD02082 | 4524 | [ETL17]sacuaUfcCfUfdGGfug | 4333 | uagAfgGfuCfaCfcAfgGfaUfa |
| | | accucuasusu | | Gfususu |
| ETD02083 | 4525 | [ETL17]suggugUfgCfCfaCfaac | 4334 | uguGfaGfuUfgUfgGfcAfcAfc |
| | | ucacasusu | | Cfasusu |
| ETD02084 | 4526 | [ETL17]sugugccAfcAfAfcucac | 4335 | uguUfgUfgAfgUfuGfuGfgCfa |
| | | aacasusu | | Cfasusu |
| ETD02085 | 4527 | [ETL17]sacaaCfUfcaCfaacuacg | 4336 | uguCfgUfaGfuUfgUfgAfgUfu |
| | | acasusu | | Gfususu |
| ETD02086 | 4528 | [ETL17]saacucAfcAfAfcuacga | 4337 | ucgGfuCfgUfaGfuUfgUfgAfg |
| | | ccgasusu | | Ufususu |
| ETD02087 | 4529 | [ETL17]sucacAfAfcuAfcGfacc | 4338 | uucCfcGfgUfcGfuAfgUfuGfu |
| | | gggaasusu | | Gfasusu |
| ETD02088 | 4530 | [ETL17]sagaaGfAfGfGfAfcgu | 4339 | ugcAfgGfaAfcGfuCfcUfcUfu |
| | | uccugcasusu | | Cfususu |
| ETD02089 | 4531 | [ETL17]sagcaCfUfUfCfUfucaa | 4340 | uugCfgGfuUfgAfaGfaAfgUfg |
| | | ccgcaasusu | | Cfususu |
| ETD02090 | 4532 | [ETL17]scacuUfCfUfUfCfaacc | 4341 | ucgUfgCfgGfuUfgAfaGfaAfg |
| | | gcacgasusu | | Ufgsusu |
| ETD02091 | 4533 | [ETL17]sacuuCfuUfCfdAAfcc | 4342 | uucGfuGfcGfgUfuGfaAfgAfa |
| | | gcacgaasusu | | Gfususu |
| ETD02092 | 4534 | [ETL17]scuucUfuCfAfdAccgc | 4343 | uguCfgUfgCfgGfuUfgAfaGfa |
| | | acgacasusu | | Afgsusu |
| ETD02093 | 4535 | [ETL17]suucuUfCfaaCfCfgcac | 4344 | ucgUfcGfuGfcGfgUfuGfaAfg |
| | | gacgasusu | | Afasusu |
| ETD02094 | 4536 | [ETL17]sucuuCfaaCfCfgCfacg | 4345 | uccGfuCfgUfgCfgGfuUfgAfa |
| | | acggasusu | | Gfasusu |
| ETD02095 | 4537 | [ETL17]suucggCfaUfCfgagaag | 4346 | uguAfcUfuCfuCfgAfuGfcCfg |
| | | uacasusu | | Afasusu |
| ETD02096 | 4538 | [ETL17]saucgAfGfAfAfGfuac | 4347 | ucgGfgAfuGfuAfcUfuCfuCfg |
| | | aucccgasusu | | Afususu |
| ETD02097 | 4539 | [ETL17]sucgaGfaAfGfdTaCfa | 4348 | uacGfgGfaUfgUfaCfuUfcUfc |
| | | ucccguasusu | | Gfasusu |
| ETD02098 | 4540 | [ETL17]sgaagUfaCfaUfCfccgu | 4349 | uguGfuAfcGfgGfaUfgUfaCfu |
| | | acacasusu | | Ufcsusu |
| ETD02099 | 4541 | [ETL17]sucuguGfccGfGfcuacu | 4350 | ucgAfaGfuAfgCfcGfgCfaCfa |
| | | ucgasusu | | Gfasusu |
| ETD02100 | 4542 | [ETL17]scuacUfuCfGfdAcugc | 4351 | ugaCfuUfgCfaGfuCfgAfaGfu |
| | | aagucasusu | | Afgsusu |
| ETD02101 | 4543 | [ETL17]sacuuCfgaCfUfgCfaag | 4352 | ucgGfaCfuUfgCfaGfuCfgAfa |
| | | uccgasusu | | Gfususu |
| ETD02102 | 4544 | [ETL17]suucgaCfUfgCfaagucc | 4353 | uguCfgGfaCfuUfgCfaGfuCfg |
| | | gacasusu | | Afasusu |
| ETD02103 | 4545 | [ETL17]suuacCfuCfUfdAcGfg | 4354 | uauGfaUfgCfcGfuAfgAfgGfu |
| | | caucauasusu | | Afasusu |

TABLE 11
Relative Human HGFAC mRNA Levels in Livers
of AAV8-TBG-h-HGFAC Mice, Study Part 1

| | | | Dose | Mean Human HGFAC mRNA |
|---|---|---|---|---|
| Group | n | Treatment | (ug) | (Relative to Group 1, Day 10) |
| 1 | 4 | PBS | | 1.00 |
| 2 | 4 | ETD02081 | 100 | 1.18 |
| 3 | 4 | ETD02082 | 100 | 0.95 |
| 4 | 4 | ETD02083 | 100 | 0.80 |
| 5 | 4 | ETD02084 | 100 | 1.09 |
| 6 | 4 | ETD02085 | 100 | 0.43 |
| 7 | 4 | ETD02086 | 100 | 0.61 |
| 8 | 4 | ETD02087 | 100 | 0.37 |
| 9 | 4 | ETD02088 | 100 | 0.12 |
| 10 | 4 | ETD02089 | 100 | 0.30 |
| 11 | 4 | ETD02090 | 100 | 0.33 |
| 12 | 4 | ETD02091 | 100 | 0.48 |

TABLE 12
Relative Human HGFAC mRNA Levels in Livers
of AAV8-TBG-h-HGFAC Mice, Study Part 2

| | | | Dose | Mean Human HGFAC mRNA |
|---|---|---|---|---|
| Group | n | Treatment | (ug) | (Relative to Group 1, Day 10) |
| 1 | 3 | PBS | | 1.00 |
| 2 | 4 | ETD02092 | 100 | 1.48 |
| 3 | 4 | ETD02093 | 100 | 1.21 |
| 4 | 4 | ETD02094 | 100 | 0.95 |
| 5 | 4 | ETD02095 | 100 | 2.41 |
| 6 | 4 | ETD02096 | 100 | 2.13 |
| 7 | 4 | ETD02097 | 100 | 1.35 |
| 8 | 4 | ETD02098 | 100 | 1.12 |
| 9 | 4 | ETD02099 | 100 | 0.91 |
| 10 | 4 | ETD02100 | 100 | 0.16 |
| 11 | 4 | ETD02101 | 100 | 0.42 |
| 12 | 4 | ETD02102 | 100 | 0.26 |
| 13 | 4 | ETD02103 | 100 | 0.23 |

### Example 8. Testing the Activity of HGFAC siRNAs ETD02381-ETD02396 in Wild Type Mice

The activities of siRNAs, namely ETD022381-ETD02396, were assessed. The siRNAs were attached to the GalNAc ligand ETL17 followed by a phosphorothioate linkage at the 5′ end of the sense strand. The siRNAs used in this Example are included in Table 13, where Nf is a 2′ fluoro-modified nucleoside, n is a 2′ O-methyl modified nucleoside, “d” is a 2′ deoxynucleoside, and “s” is a phosphorothioate linkage. The base sequences for each siRNA, with and without the 3′ UU extension, are shown in Table 14.

On Day 0 female mice (strain ICR) were given a subcutaneous injection of a single 200 μg dose of a GalNAc-conjugated siRNA or PBS as vehicle control. A mouse-specific siRNA ETD02258 was used as a positive control (Sense strand, [ETL 17]saugcUfuUfGfAfugaaacacgasusu [SEQ ID NO: 4822]; antisense strand, usCfsgUfgUfuUfcAfuCfaAfaGfcAfususu [SEQ ID NO: 4823]). On Day 11 after subcutaneous injection, mice were euthanized and a liver sample from each was collected and placed in RNAlater (ThermoFisher Cat #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver HGFAC mRNA were assessed by RT-qPCR in triplicate on a QuantStudio™ 6 Pro Real-Time PCR System using TaqMan assays for mouse HGFAC (ThermoFisher, assay #Mm00469483 ml) and the mouse housekeeping gene PPIA (ThermoFisher, assay #Mm02342430_g1) and PerfeCTa® qPCR FastMix®, Low ROX™ (VWR, Catalog #101419-222). Data were normalized to the level in animals receiving PBS. The results of part 1 of the study are shown in Table 15.

TABLE 13
Example siRNA Sequences

| siRNA | SEQ ID | Sense Strand Sequence (5′-3′) | SEQ ID | Antisense Strand Sequence |
|---|---|---|---|---|
| Name | NO: | with GalNAc moiety | NO: | (5′-3′) |
| ETD02381 | 4546 | [ETL17]scggcAfcAfGfAfGfaaa | 4483 | usAfsaGfcAfuUfuCfuCfuGfu |
| | | ugcuuasusu | | GfcCfgsusu |
| ETD02382 | 4547 | [ETL17]sggcacAfGfAfGfAfaa | 4484 | usAfsaAfgCfaUfuUfcUfcUfg |
| | | | | UfgCfcsusu |
| ETD02383 | 4548 | [ETL17]sgcacAfGfAfGfAfaau | 4485 | usCfsaAfaGfcAfuUfuCfuCfu |
| | | gcuuugasusu | | GfuGfcsusu |
| ETD02384 | 4549 | [ETL17]scacaGfAfGfAfAfaugc | 4486 | usUfscAfaAfgCfaUfuUfcUfc |
| | | uuugaasusu | | UfgUfgsusu |
| ETD02385 | 4550 | [ETL17]sacagAfGfAfAfAfugc | 4487 | usAfsuCfaAfaGfcAfuUfuCfu |
| | | uuugauasusu | | CfuGfususu |
| ETD02386 | 4551 | [ETL17]scagaGfaAfAfdTgCfu | 4488 | usCfsaUfcAfaAfgCfaUfuUfc |
| | | uugaugasusu | | UfcUfgsusu |
| ETD02387 | 4552 | [ETL17]sagagAfAfauGfcuuuga | 4489 | usUfscAfuCfaAfaGfcAfuUfu |
| | | ugaasusu | | CfuCfususu |
| ETD02388 | 4553 | [ETL17]sgaaaUfgCfUfUfUfga | 4490 | usGfsuCfuCfaUfcAfaAfgCfa |
| | | ugagacasusu | | UfuUfcsusu |
| ETD02389 | 4554 | [ETL17]sacauaCfagCfUfugucu | 4491 | usCfsuCfaGfaCfaAfgCfuGfu |
| | | gagasusu | | AfuGfususu |
| ETD02390 | 4555 | [ETL17]sugauCfCfggCfUfgaag | 4492 | usUfscUfuCfuUfcAfgCfcGfg |
| | | aagaasusu | | AfuCfasusu |
| ETD02391 | 4556 | [ETL17]sgaucCfggCfUfgaagaa | 4493 | usUfsuCfuUfcUfuCfaGfcCfg |
| | | gaaasusu | | GfaUfcsusu |
| ETD02392 | 4557 | [ETL17]saggacAfcAfAfgugcca | 4494 | usAfsuCfuGfgCfaCfuUfgUfg |
| | | gauasusu | | UfcCfususu |
| ETD02393 | 4558 | [ETL17]sccaacuAfuGfuGfgacu | 4495 | usUfscCfaGfuCfcAfcAfuAfg |
| | | ggaasusu | | UfuGfgsusu |
| ETD02394 | 4559 | [ETL17]scaacUfaUfgUfggacug | 4496 | usAfsuCfcAfgUfcCfaCfaUfa |
| | | gauasusu | | GfuUfgsusu |
| ETD02395 | 4560 | [ETL17]scuauGfuGfgAfcuggau | 4497 | usUfsuGfaUfcCfaGfuCfcAfc |
| | | caaasusu | | AfuAfgsusu |
| ETD02396 | 4561 | [ETL17]suaugUfggaCfUfggauc | 4498 | usGfsuUfgAfuCfcAfgUfcCfa |
| | | aacasusu | | CfaUfasusu |

TABLE 14
Example siRNA BASE Sequences

| | SEQ | Sense Strand | SEQ | Antisense Strand |
|---|---|---|---|---|
| siRNA | ID | Base Sequence | ID | Base Sequence |
| Name | NO: | (5′ to 3′) | NO: | (5′ to 3′) |
| ETD02381 | 4585 | CGGCACAGAGAAAUGCUUAUU | 4640 | UAAGCAUUUCUCUGUGCCGUU |
| ETD02382 | 4586 | GGCACAGAGAAAUGCUUUAUU | 4641 | UAAAGCAUUUCUCUGUGCCUU |
| ETD02383 | 4587 | GCACAGAGAAAUGCUUUGAUU | 4642 | UCAAAGCAUUUCUCUGUGCUU |
| ETD02384 | 4588 | CACAGAGAAAUGCUUUGAAUU | 4643 | UUCAAAGCAUUUCUCUGUGUU |
| ETD02385 | 4589 | ACAGAGAAAUGCUUUGAUAUU | 4644 | UAUCAAAGCAUUUCUCUGUUU |
| ETD02386 | 4590 | CAGAGAAATGCUUUGAUGAUU | 4645 | UCAUCAAAGCAUUUCUCUGUU |
| ETD02387 | 4591 | AGAGAAAUGCUUUGAUGAAUU | 4646 | UUCAUCAAAGCAUUUCUCUUU |
| ETD02388 | 4592 | GAAAUGCUUUGAUGAGACAUU | 4647 | UGUCUCAUCAAAGCAUUUCUU |
| ETD02389 | 4593 | ACAUACAGCUUGUCUGAGAUU | 4648 | UCUCAGACAAGCUGUAUGUUU |
| ETD02390 | 4594 | UGAUCCGGCUGAAGAAGAAUU | 4649 | UUCUUCUUCAGCCGGAUCAUU |
| ETD02391 | 4595 | GAUCCGGCUGAAGAAGAAAUU | 4650 | UUUCUUCUUCAGCCGGAUCUU |
| ETD02392 | 4596 | AGGACACAAGUGCCAGAUAUU | 4651 | UAUCUGGCACUUGUGUCCUUU |
| ETD02393 | 4597 | CCAACUAUGUGGACUGGAAUU | 4652 | UUCCAGUCCACAUAGUUGGUU |
| ETD02394 | 4598 | CAACUAUGUGGACUGGAUAUU | 4653 | UAUCCAGUCCACAUAGUUGUU |
| ETD02395 | 4599 | CUAUGUGGACUGGAUCAAAUU | 4654 | UUUGAUCCAGUCCACAUAGUU |
| ETD02396 | 4600 | UAUGUGGACUGGAUCAACAUU | 4655 | UGUUGAUCCAGUCCACAUAUU |
| | | Sense Strand | | Antisense Strand |
| | SEQ | Base Sequence | SEQ | Base Sequence |
| siRNA | ID | (5′ to 3′), | ID | (5′ to 3′), |
| Name | NO: | without 3′ overhangs | NO: | without 3′ overhangs |
| ETD02381 | 4601 | CGGCACAGAGAAAUGCUUA | 4656 | UAAGCAUUUCUCUGUGCCG |
| ETD02382 | 4602 | GGCACAGAGAAAUGCUUUA | 4657 | UAAAGCAUUUCUCUGUGCC |
| ETD02383 | 4603 | GCACAGAGAAAUGCUUUGA | 4658 | UCAAAGCAUUUCUCUGUGC |
| ETD02384 | 4604 | CACAGAGAAAUGCUUUGAA | 4659 | UUCAAAGCAUUUCUCUGUG |
| ETD02385 | 4605 | ACAGAGAAAUGCUUUGAUA | 4660 | UAUCAAAGCAUUUCUCUGU |
| ETD02386 | 4606 | CAGAGAAATGCUUUGAUGA | 4661 | UCAUCAAAGCAUUUCUCUG |
| ETD02387 | 4607 | AGAGAAAUGCUUUGAUGAA | 4662 | UUCAUCAAAGCAUUUCUCU |
| ETD02388 | 4608 | GAAAUGCUUUGAUGAGACA | 4663 | UGUCUCAUCAAAGCAUUUC |
| ETD02389 | 4609 | ACAUACAGCUUGUCUGAGA | 4664 | UCUCAGACAAGCUGUAUGU |
| ETD02390 | 4610 | UGAUCCGGCUGAAGAAGAA | 4665 | UUCUUCUUCAGCCGGAUCA |
| ETD02391 | 4611 | GAUCCGGCUGAAGAAGAAA | 4666 | UUUCUUCUUCAGCCGGAUC |
| ETD02392 | 4612 | AGGACACAAGUGCCAGAUA | 4667 | UAUCUGGCACUUGUGUCCU |
| ETD02393 | 4613 | CCAACUAUGUGGACUGGAA | 4668 | UUCCAGUCCACAUAGUUGG |
| ETD02394 | 4614 | CAACUAUGUGGACUGGAUA | 4669 | UAUCCAGUCCACAUAGUUG |
| ETD02395 | 4615 | CUAUGUGGACUGGAUCAAA | 4670 | UUUGAUCCAGUCCACAUAG |
| ETD02396 | 4616 | UAUGUGGACUGGAUCAACA | 4671 | UGUUGAUCCAGUCCACAUA |

TABLE 15
Relative Mouse HGFAC mRNA Levels in Livers of ICR Mice

| | | | Dose | Mean Mouse HGFAC mRNA |
|---|---|---|---|---|
| Group | n | Treatment | (ug) | (Relative to Group 1, Day 11) |
| 1 | 4 | PBS | | 1.00 |
| 2 | 4 | ETD02258 | 200 | 0.06 |
| 3 | 4 | ETD02381 | 200 | 0.18 |
| 4 | 4 | ETD02382 | 200 | 0.17 |
| 5 | 4 | ETD02383 | 200 | 0.11 |
| 6 | 4 | ETD02384 | 200 | 0.15 |
| 7 | 4 | ETD02385 | 200 | 0.12 |
| 8 | 4 | ETD02386 | 200 | 0.13 |
| 9 | 4 | ETD02387 | 200 | 0.18 |
| 10 | 4 | ETD02388 | 200 | 0.31 |
| 11 | 4 | ETD02389 | 200 | 1.40 |
| 12 | 4 | ETD02390 | 200 | 0.81 |
| 13 | 4 | ETD02391 | 200 | 0.46 |
| 14 | 4 | ETD02392 | 200 | 0.41 |
| 15 | 4 | ETD02393 | 200 | 0.21 |
| 16 | 4 | ETD02394 | 200 | 0.32 |
| 17 | 4 | ETD02395 | 200 | 0.08 |
| 18 | 4 | ETD02396 | 200 | 0.41 |

### Example 9: Oligonucleotide Synthesis

Oligonucleotides such as siRNAs may be synthesized according to phosphoramidite technology on a solid phase. For example, a K&A oligonucleotide synthesizer may be used. Syntheses may be performed on a solid support made of controlled pore glass (CPG, 500 Å or 600 Å, obtained from AM Chemicals, Oceanside, CA, USA). All 2′-OMe and 2′-F phosphoramidites may be purchased from Hongene Biotech (Union City, CA, USA). All phosphoramidites may be dissolved in anhydrous acetonitrile (100 mM) and molecular sieves (3 Å) may be added. 5-Benzylthio-11H-tetrazole (BTT, 250 mM in acetonitrile) or 5-Ethylthio-1H-tetrazole (ETT, 250 mM in acetonitrile) may be used as activator solution. Coupling times may be 9-18 min (e.g. with a GalNAc such as ETL17), 6 min (e.g. with 2′OMe and 2′F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile may be employed.

After solid phase synthesis, the dried solid support may be treated with a 1:1 volume solution of 40 wt. % methylamine in water and 28% ammonium hydroxide solution (Aldrich) for two hours at 30° C. The solution may be evaporated and the solid residue may be reconstituted in water and purified by anionic exchange HPLC using a TKSgel SuperQ-5PW 13u column. Buffer A may be 20 mM Tris, 5 mM EDTA, pH 9.0 and contained 20% Acetonitrile and buffer B may be the same as buffer A with the addition of 1 M sodium chloride. UV traces at 260 nm may be recorded. Appropriate fractions may be pooled then desalted using Sephadex G-25 medium.

Equimolar amounts of sense and antisense strand may be combined to prepare a duplex. The duplex solution may be prepared in 0.1×PBS (Phosphate-Buffered Saline, 1×, Gibco). The duplex solution may be annealed at 95° C. for 5 min, and cooled to room temperature slowly. Duplex concentration may be determined by measuring the solution absorbance on a UV-Vis spectrometer at 260 nm in 0.1×PBS. For some experiments, a conversion factor may be calculated from an experimentally determined extinction coefficient

### Example 10: GalNAc Ligand for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. Reagents for GalNAc conjugation to oligonucleotides are shown in Table 16.

TABLE 16
GalNAc Conjugation Reagents

| Type of | |
|---|---|
| conjugation | Structure |
| Solid phase 3′ attachment where squiggly line is rest of oligonucleotide chain and right-most OH is where attachment to solid phase is. | 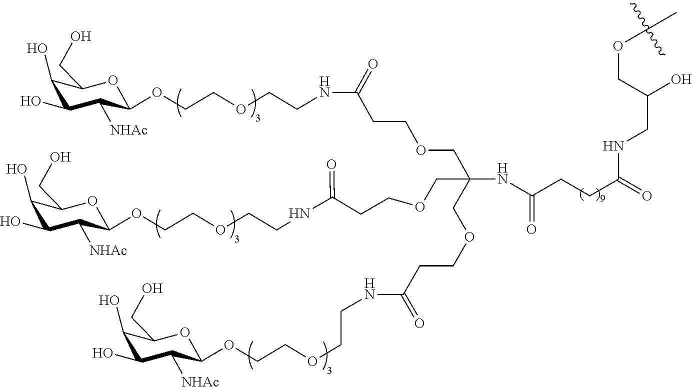 |
| | This GalNAc ligand may be referred to as “GalNAc23” or “GalNAc#23.” |
| Solid phase 5′ attachment phosphoramidite | 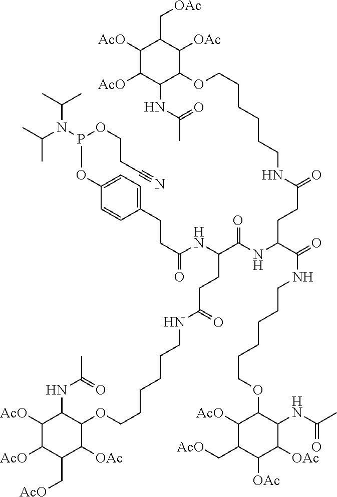 [SMILES: CC(=O)NC1C(OCCCCCCNC(=O)CCC(NC(=O)CCc2ccc(OP(OCCC#N)N(C(C)C)C(C)C)cc2)C(=O)NC(CCC(=O)NCCCCCCOC2C(NC(C)=O)C(OC(C)=O)C(OC(C)=O)C(COC(C)=O)C2OC(C)=O)C(=O)NCCCCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solid phase 5′ attachment Phosphoramidite | 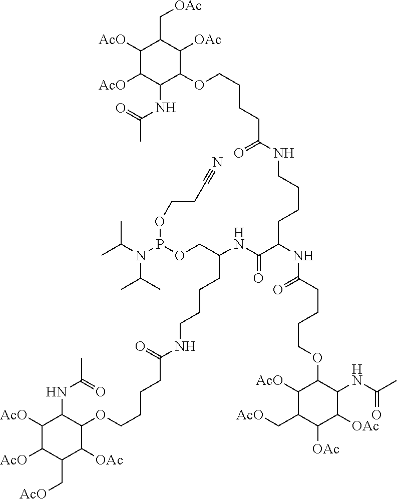 [SMILES: CC(=O)NC1C(OCCCCC(=O)NCCCCC(COP(OCCC#N)N(C(C)C)C(C)C)NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(OC(C)=O)C(COC(C)=O)C(OC(C)=O)C1OC(C)=O] |
| Solution phase Carboxylic acid for amide coupling anywhere on oligonucleotide | 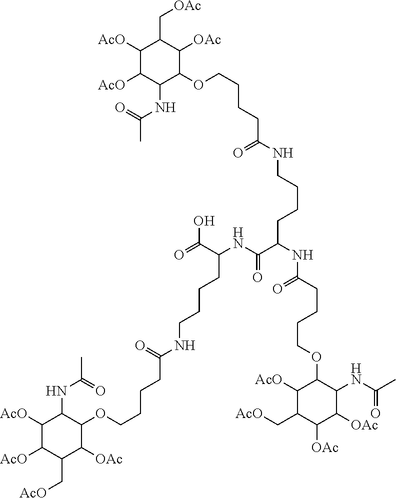 [SMILES: CC(=O)NC1C(OCCCCC(=O)NCCCCC(NC(=O)C(CCCCNC(=O)CCCCOC2C(NC(C)=O)C(OC(C)=O)C(OC(C)=O)C(COC(C)=O)C2OC(C)=O)NC(=O)CCCCOC2C(NC(C)=O)C(OC(C)=O)C(OC(C)=O)C(COC(C)=O)C2OC(C)=O)C(=O)O)C(OC(C)=O)C(COC(C)=O)C(OC(C)=O)C1OC(C)=O] |
| | Where Ac is an acetyl group or other hydroxyl protecting group that can be removed |
| | under basic, acid or reducing conditions. |

In solution phase conjugation, the oligonucleotide sequence-including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between

Non-limiting examples of reagents for oligonucleotide synthesis to incorporate an amino group include:
5′ attachment:
6-(4-Monomethoxytritylamino)hexyl-(2-cyanoethyl)-(N,N-diisopropyl)-phosphoramidite CAS Number: 114616-27-2
5′-Amino-Modifier TEG CE-Phosphoramidite
10-(O-trifluoroacetamido-N-ethyl)-triethyleneglycol-1-[(2-cyanoethyl)-(N,N-diisopropyl)]-phosphoramidite
3′ attachment:
3′-Amino-Modifier Serinol CPG
3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
Amino-Modifier Serinol Phosphoramidite
3-Dimethoxytrityloxy-2-(3-(fluorenylmethoxycarbonylamino)propanamido)propyl-1-O-(2-cyanoethyl)-(N,N-diisopropyl)-phosphoramidite

### Internal (Base Modified):

Amino-Modifier C6 dT
5′-Dimethoxytrityl-5-[N-(trifluoroacetylaminohexyl)-3-acrylimido]-2′-deoxyUridine,3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

### Example 10: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 17.

TABLE 17
GalNAc Conjugation Reagent

| Type of | |
|---|---|
| conjugation | Structure |
| Solid phase 5′ attachment phosphora- midite | 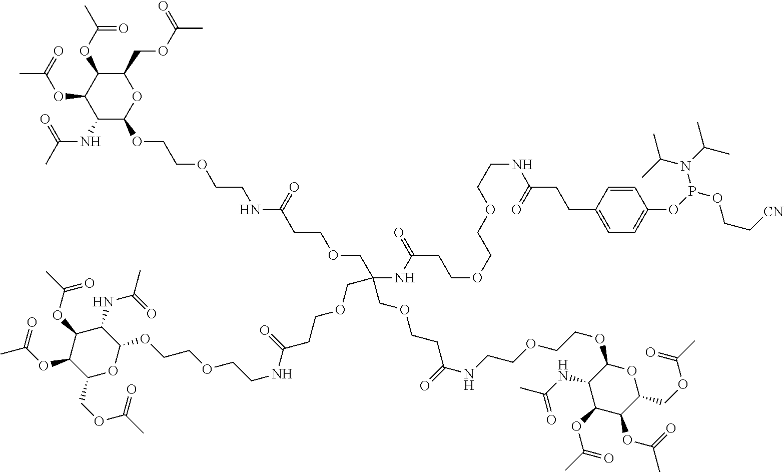 [SMILES: CC(=O)N[C@@H]1[C@H](OCCOCCNC(=O)CCOCC(COCCC(=O)NCCOCCO[C@H]2O[C@H](COC(C)=O)[C@@H](OC(C)=O)[C@@H](OC(C)=O)[C@@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCc2ccc(OP(OCCC#N)N(C(C)C)C(C)C)cc2)O[C@H](COC(C)=O)[C@@H](OC(C)=O)[C@H]1OC(C)=O] |

The following includes examples of synthesis reactions used to create a GalNAc moiety:

### Scheme for the Preparation of NAcegal-Linker-TMSOTf

### General Procedure for Preparation of Compound 2A

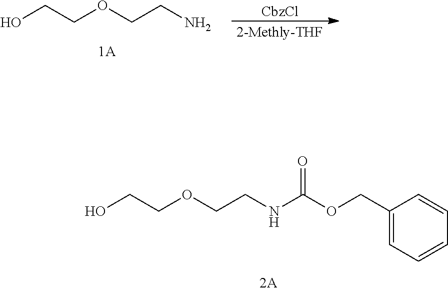

To a solution of Compound 1A (500 g, 4.76 mol, 476 mL) in 2-Methyl-THF (2.00 L) is added CbzCl (406 g, 2.38 mol, 338 mL) in 2-Methyl-THF (750 mL) dropwise at 0° C. The mixture is stirred at 25° C. for 2 hrs under N₂ atmosphere. TLC (DCM: MeOH=20:1, PMA) may indicate CbzCl is consumed completely and one new spot (R_{f}=0.43) formed. The reaction mixture is added HCl/EtOAc (1 N, 180 mL) and stirred for 30 mins, white solid is removed by filtration through celite, the filtrate is concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

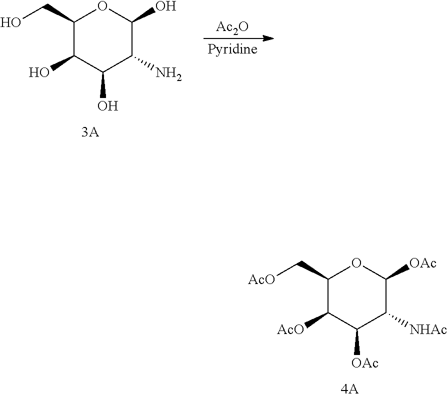 [SMILES: "Ac2O" ; N[C@@H]1[C@@H](O)[C@@H](O)[C@@H](CO)O[C@H]1O ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) is added acetyl acetate (4.73 kg, 46.4 mol, 4.34 L) dropwise at 0° C. under N₂ atmosphere. The mixture is stirred at 25° C. for 16 hrs under N₂ atmosphere. TLC (DCM: MeOH=20:1, PMA) indicated Compound 3A is consumed completely and two new spots (R_{f}=0.35) formed. The reaction mixture is added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. ¹H NMR: δ 7.90 (d, J 9.29 Hz, 1H), 5.64 (d, J 8.78 Hz, 1H), 5.26 (d, J 3.01 Hz, 1H), 5.06 (dd, J 11.29, 3.26 Hz, 1H), 4.22 (t, J 6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5A

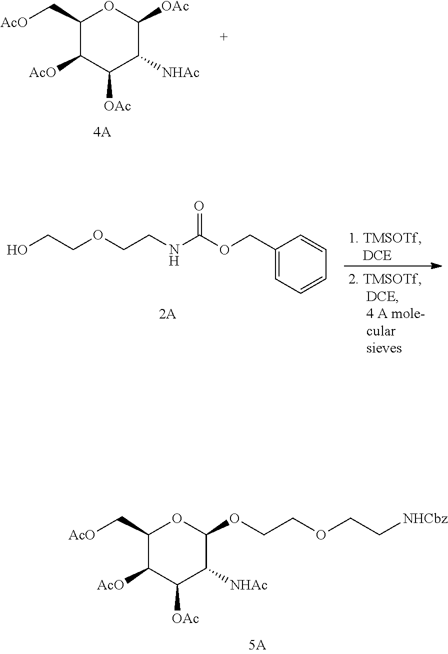 [SMILES: CC(=O)N[C@H]1[C@H](OC(C)=O)O[C@H](COC(C)=O)[C@H](OC(C)=O)[C@@H]1OC(C)=O ; O=C(NCCOCCO)OCc1ccccc1 ; CC(=O)N[C@H]1[C@H](OCCOCCNC(=O)OCc2ccccc2)O[C@H](COC(C)=O)[C@H](OC(C)=O)[C@@H]1OC(C)=O]

To a solution of Compound 4A (300 g, 771 mmol) in DCE (1.50 L) is added TMSOTf (257 g, 1.16 mol, 209 mL) and stirred for 2 hrs at 60° C., and then stirred for 1 hr at 25° C. Compound 2A (203 g, 848 mmol) is dissolved in DCE (1.50 L) and added 4 Å powder molecular sieves (150 g) stirring for 30 mins under N₂ atmosphere. Then the solution of Compound 4A in DCE is added dropwise to the mixture at 0° C. The mixture is stirred at 25° C. for 16 hrs under N₂ atmosphere. TLC (DCM: MeOH=25:1, PMA) indicated Compound 4A is consumed completely and new spot (R_{f}=0.24) formed. The reaction mixture is filtered and washed with sat. NaHCO₃ (2.00 L), water (2.00 L) and sat. brine (2.00 L). The organic layer is dried over anhydrous Na₂SO₄, filtered and concentrated under reduced pressure to give a residue. The residue is triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J 9.29 Hz, 1H), 7.20-7.42 (m, 6H), 5.21 (d, J 3.26 Hz, 1H), 4.92-5.05 (m, 3H), 4.55 (d, J 8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J 5.77 Hz, 2H), 2.10 (s, 3H), 1.99 (s, 3H), 1.89 (s, 3H), 1.77 (s, 3H).

### General Procedure for Preparation of NAcegal-Linker-Tosylate Salt

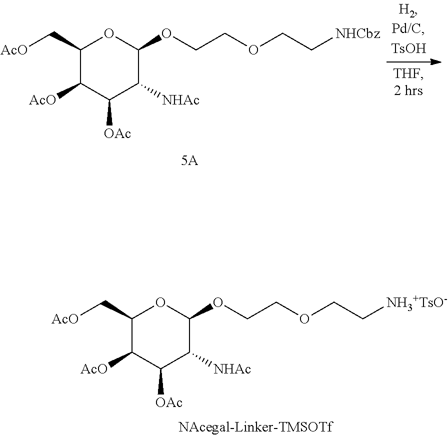

To a solution of Compound 5A (200 g, 352 mmol) in THF (1.0 L) is added dry Pd/C (15.0 g, 10% purity) and TsOH (60.6 g, 352 mmol) under N₂ atmosphere. The suspension is degassed under vacuum and purged with H₂ several times. The mixture is stirred at 25° C. for 3 hrs under H₂ (45 psi) atmosphere. TLC (DCM: MeOH=10:1, PMA) indicated Compound 5A is consumed completely and one new spot (R_{f}=0.04) is formed. The reaction mixture is filtered and concentrated (≤40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This is repeated 3 times with anhydrous DCM (500 mL) dilutions and concentration to give NAcegal-Linker-TMSOTf (205 g, 95.8% yield, TsOH salt) as a foamy white solid. ¹H NMR: δ 7.91 (d, J 9.03 Hz, 1H), 7.53-7.86 (m, 2H), 7.49 (d, J 8.03 Hz, 2H), 7.13 (d, J 8.03 Hz, 2H), 5.22 (d, J 3.26 Hz, 1H), 4.98 (dd, J 11.29, 3.26 Hz, 1H), 4.57 (d, J 8.53 Hz, 1H), 3.99-4.05 (m, 3H), 3.87-3.94 (m, 1H), 3.79-3.85 (m, 1H), 3.51-3.62 (m, 5H), 2.96 (br t, J 5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

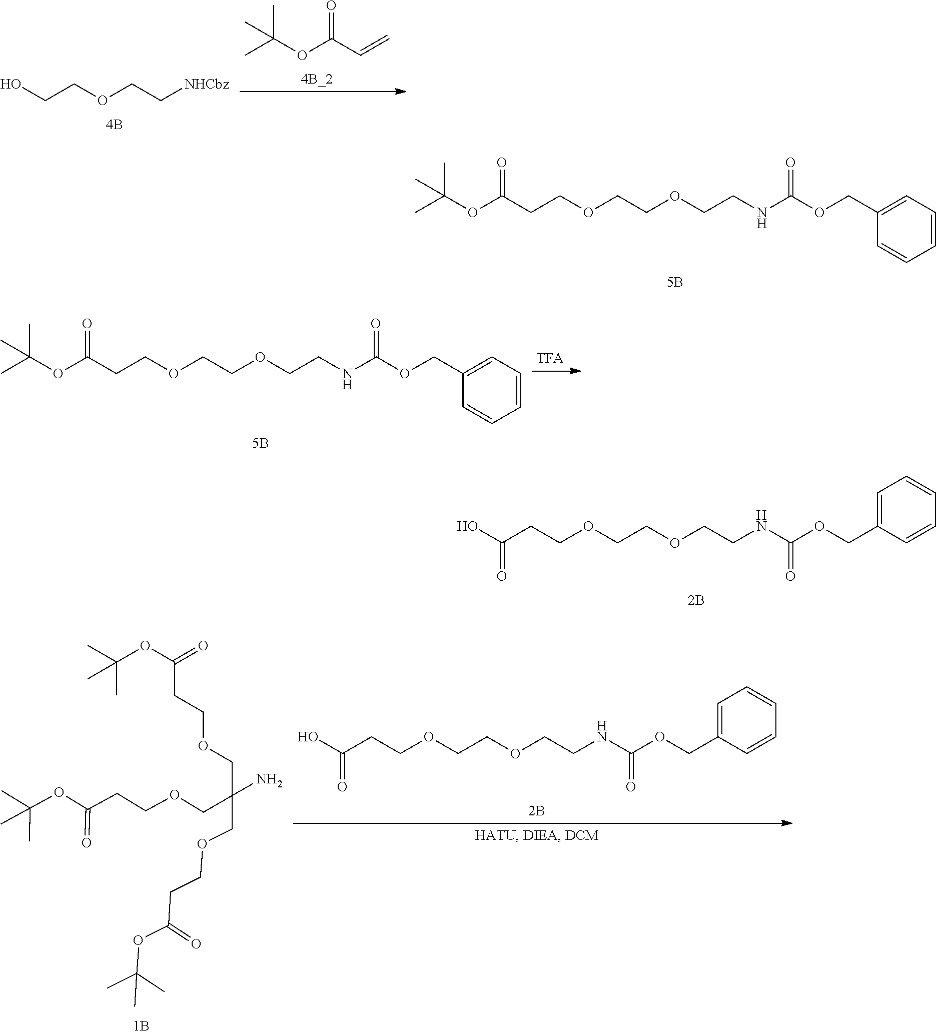

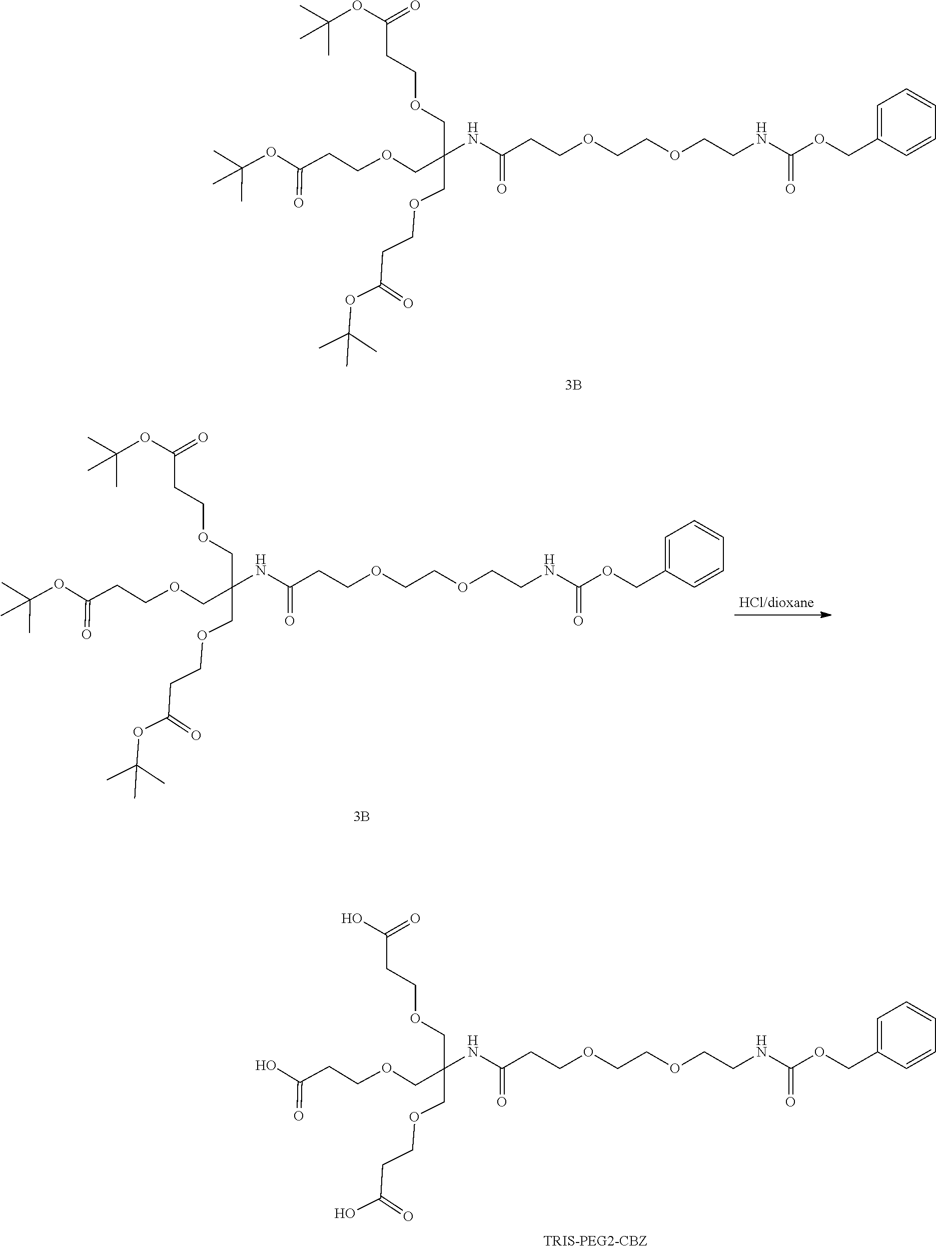 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

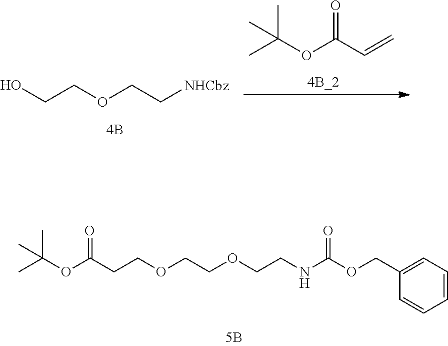

To a solution of Compound 4B (400 g, 1.67 mol, 1.00 eq) and NaOH (10 M, 16.7 mL, 0.10 eq) in THF (2.00 L) is added Compound 4B_2 (1.07 kg, 8.36 mol, 1.20 L, 5.00 eq), the mixture is stirred at 30° C. for 2 hrs. LCMS showed the desired MS is given. Five batches of solution are combined to one batch, then the mixture is diluted with water (6.00 L), extracted with ethyl acetate (3.00 L*3), the combined organic layer is washed with brine (3.00 L), dried over Na₂SO₄, filtered and concentrated under vacuum. The crude is purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General procedure for preparation of 3-oxo-1-phenyl-2,7,10-trioxa-4-azatridecan-13-oic acid (Compound 2B below)

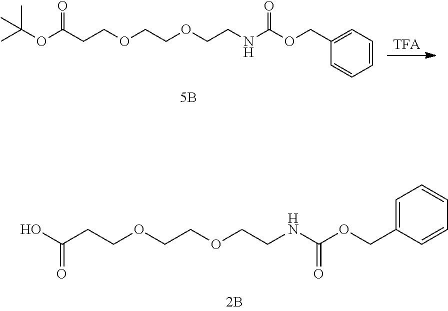 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) is added TFA (1.43 kg, 12.5 mol, 928 mL, 6.22 eq), the mixture is stirred at 25° C. for 3 hrs. LCMS showed the desired MS is given. The mixture is diluted with DCM (5.00 L), washed with water (3.00 L*3), brine (2.00 L), the combined organic layer is dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

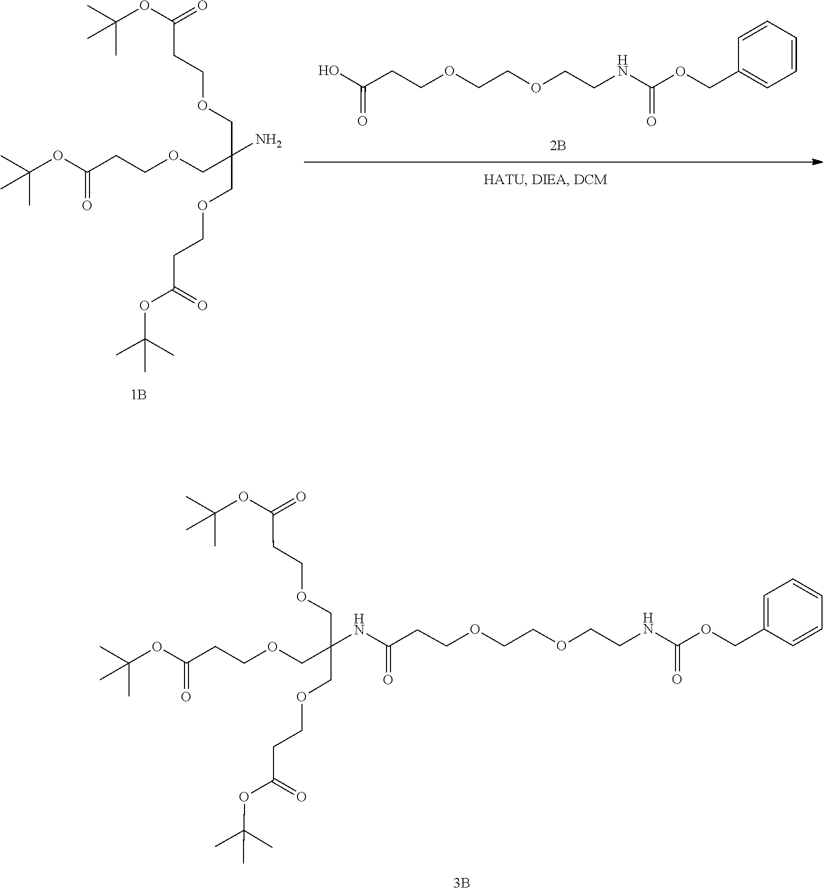 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) is added HATU (570 g, 1.50 mol, 1.50 eq) and DIEA (258 g, 2.00 mol, 348 mL, 2.00 eq) at 0° C., the mixture is stirred at 0° C. for 30 min, then Compound 1B (606 g, 1.20 mol, 1.20 eq) is added, the mixture is stirred at 25° C. for 1 hr. LCMS showed desired MS is given. The mixture is combined to one batch, then the mixture is diluted with DCM (5.00 L), washed with 1 N HCl aqueous solution (2.00 L*2), then the organic layer is washed with saturated Na₂CO₃ aqueous solution (2.00 L*2) and brine (2.00 L), the organic layer is dried over Na₂SO₄, filtered and concentrated under vacuum to give Compound 3B (3.88 kg, crude) as yellow oil.

### General Procedure for Preparation of TRIS-PEG2-CBZ

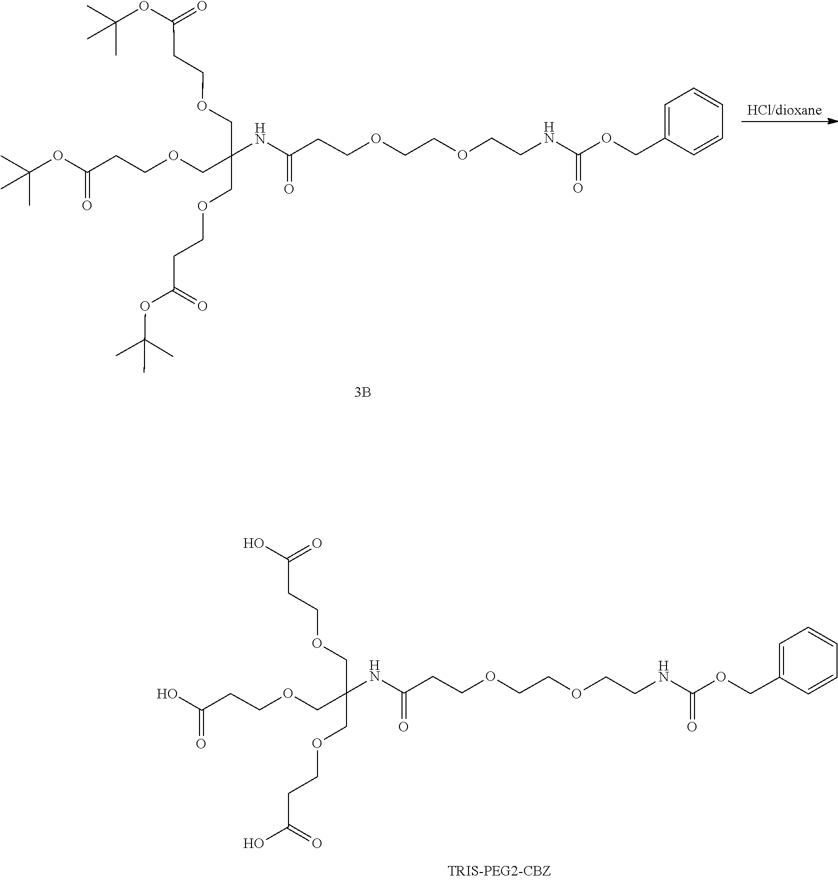 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) is stirred at 25° C. for 2 hrs. LCMS showed the desired MS is given. The mixture is concentrated under vacuum to give a residue. Then the combined residue is diluted with DCM (5.00 L), adjusted to pH=8 with 2.5 M NaOH aqueous solution, and separated. The aqueous phase is extracted with DCM (3.00 L) again, then the aqueous solution is adjusted to pH=3 with 1 N HCl aqueous solution, then extracted with DCM (5.00 L*2), the combined organic layer is washed with brine (3.00 L), dried over Na₂SO₄, filtered and concentrated under vacuum. The crude is purified by column chromatography (SiO₂, DCM:MeOH=0:1-12:1, 0.1% HOAc, R_{f}=0.4). The residue is diluted with DCM (5.00 L), adjusted to pH=8 with 2.5 M NaOH aqueous solution, separated, the aqueous solution is extracted with DCM (3.00 L) again, then the aqueous solution is adjusted to pH=3 with 6 N HCl aqueous solution, extracted with DCM:MeOH=10:1 (5.00 L*2), the combined organic layer is washed with brine (2.00 L), dried over Na₂SO₄, filtered and concentrated under vacuum to give a residue. Then the residue is diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Phosph 8c

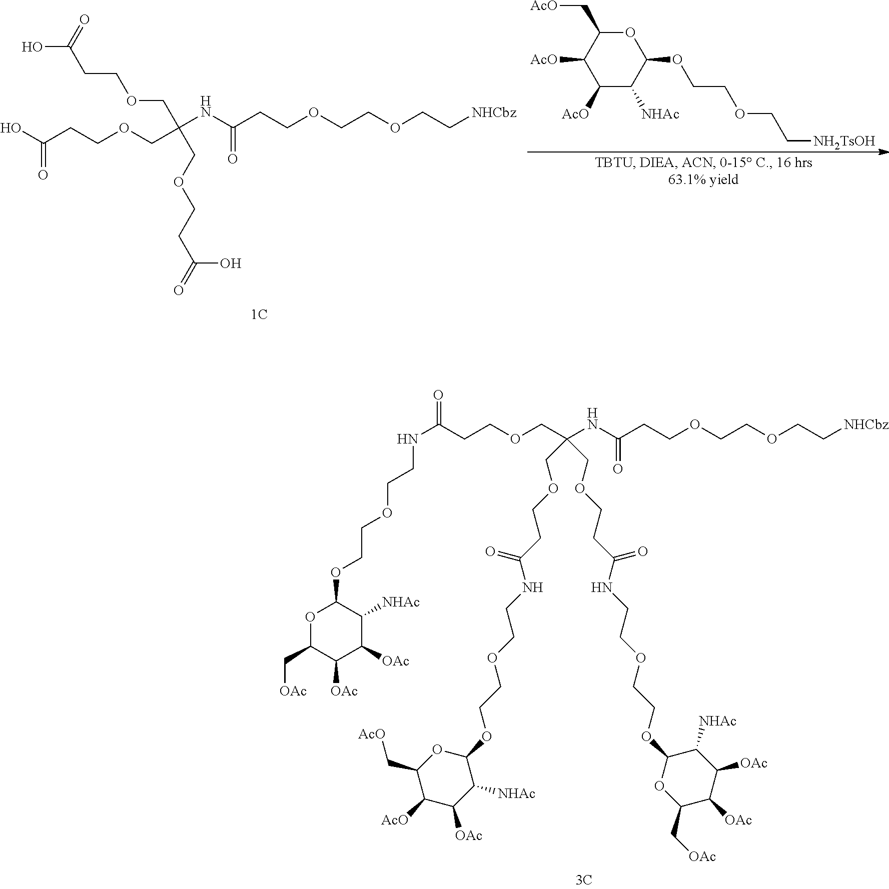

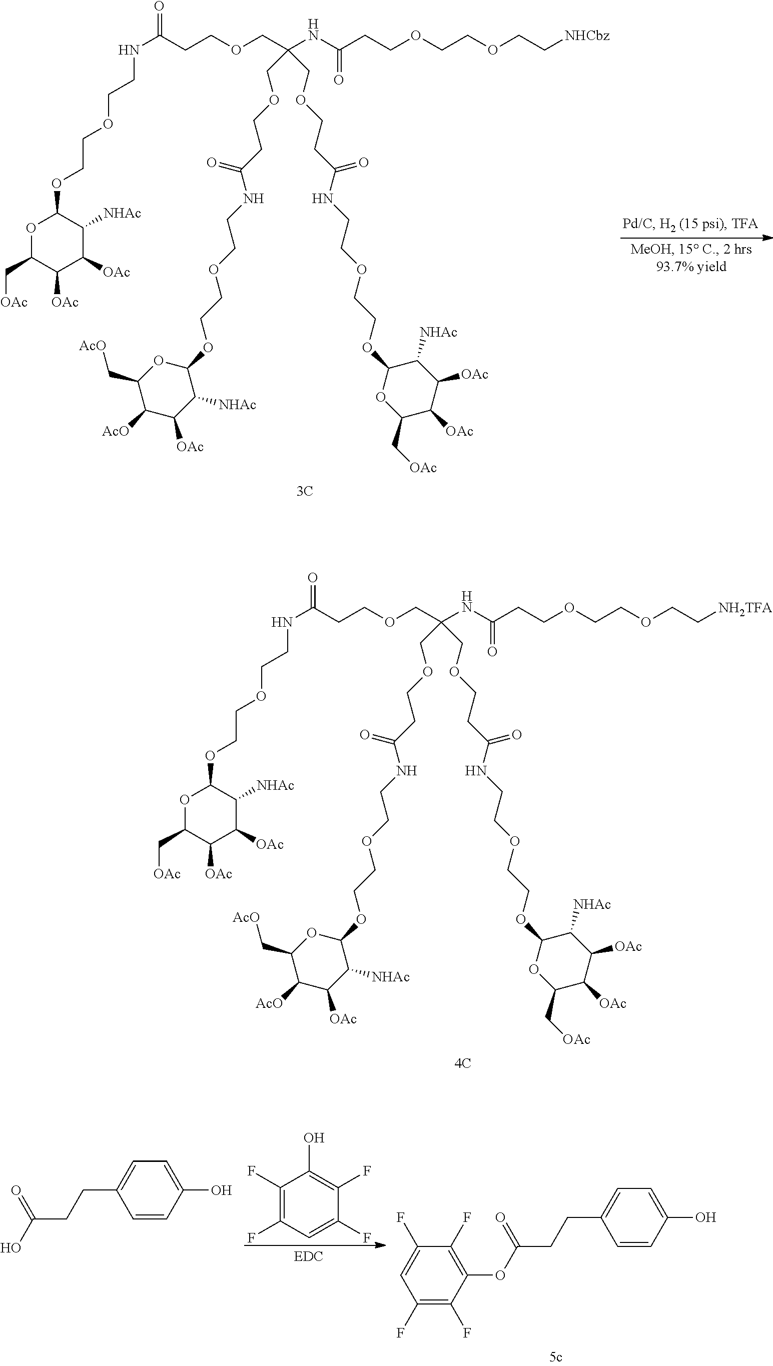

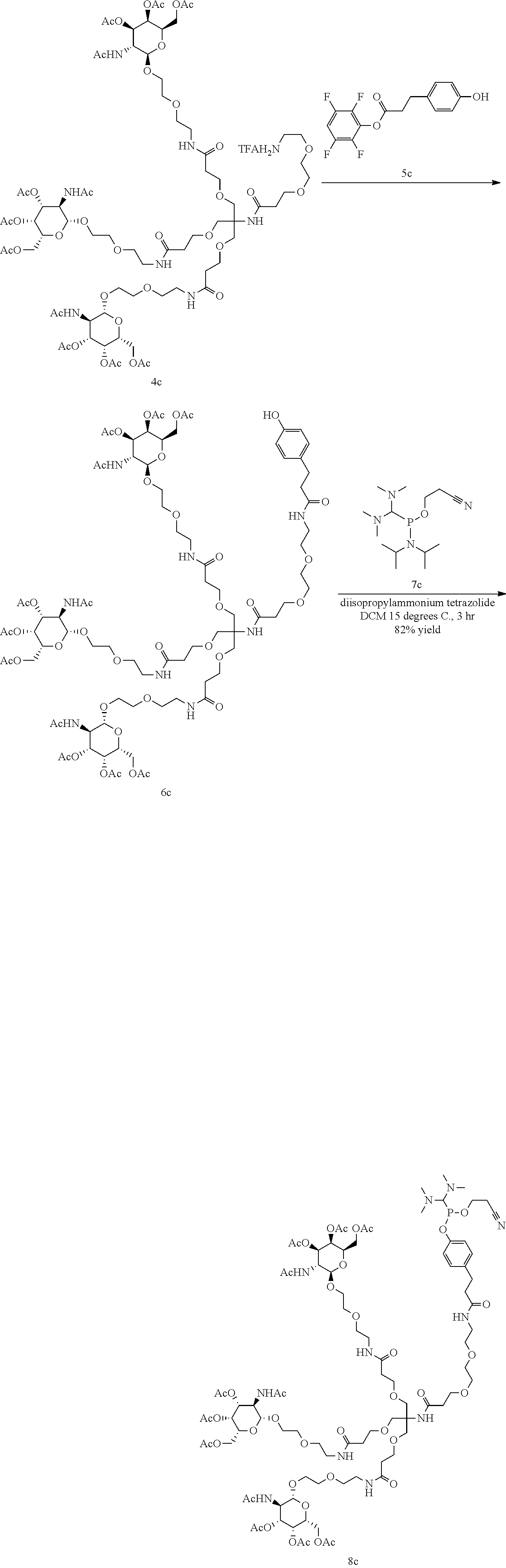

### TriGNal-TRIS-Peg2-Phosph 8c

### General Procedure for Preparation of Compound 3C

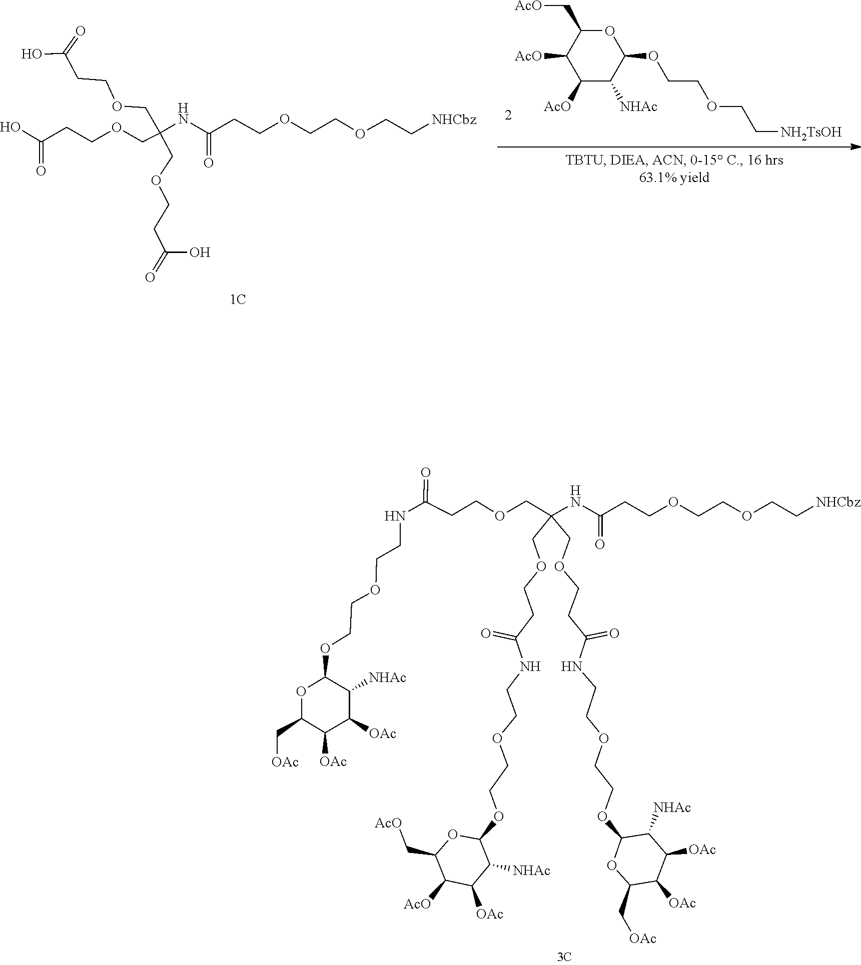

To a solution of Compound 1C (155 g, 245 mmol, 1.00 eq) in ACN (1500 mL) is added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture is stirred at 15° C. for 16 hrs. LCMS showed the desired MS is given. The mixture is concentrated under vacuum to give a residue, then the mixture is diluted with DCM (2000 mL), washed with 1 N HCl aqueous solution (700 mL*2), then saturated NaHCO₃ aqueous solution (700 mL*2) and concentrated under vacuum. The crude is purified by column chromatography to give Compound 3C (304 g, 155 mmol, 63.1% yield, 96.0% purity) as a yellow solid.

### General Procedure for Preparation of Compound 4C

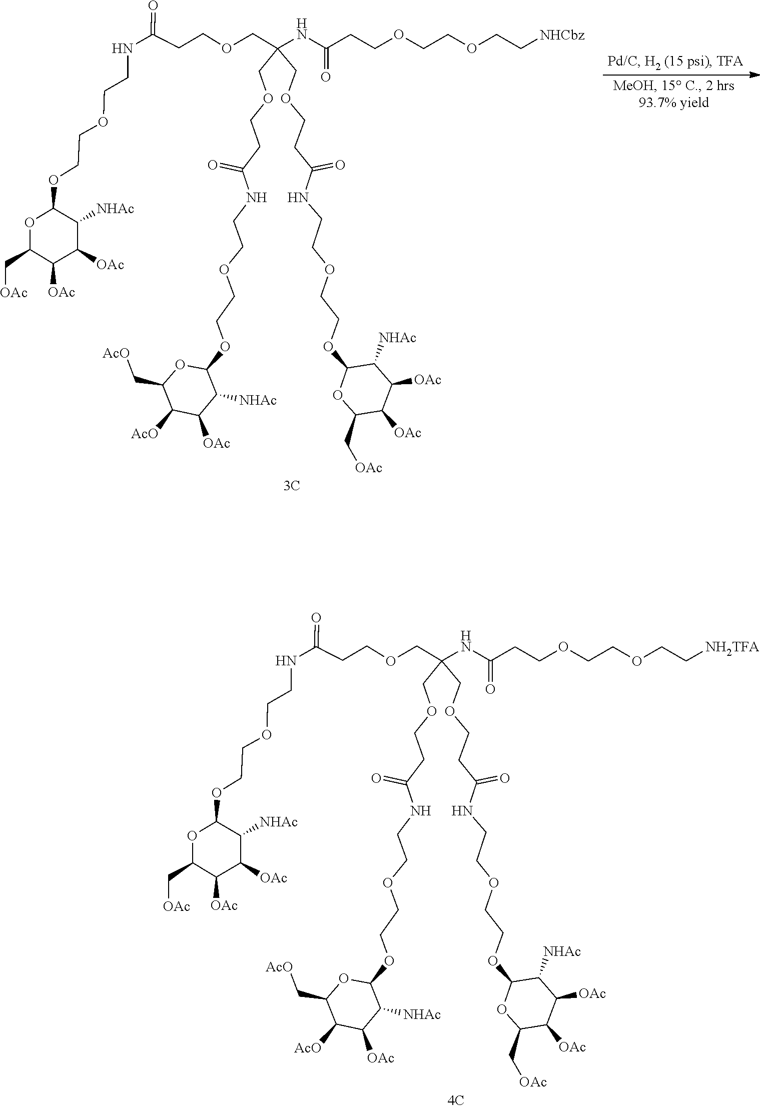

Two batches solution of Compound 3C (55.0 g, 29.2 mmol, 1.00 eq) in MeOH (1600 mL) is added Pd/C (6.60 g, 19.1 mmol, 10.0% purity) and TFA (3.34 g, 29.2 mmol, 2.17 mL, 1.00 eq), the mixture is degassed under vacuum and purged with H₂. The mixture is stirred under H₂ (15 psi) at 15° C. for 2 hours. LCMS showed the desired MS is given. The mixture is filtered and the filtrate is concentrated under vacuum to give Compound 4C (106 g, 54.8 mmol, 93.7% yield, 96.2% purity, TFA) as a white solid.

### General Procedure for Preparation of Compound 5C

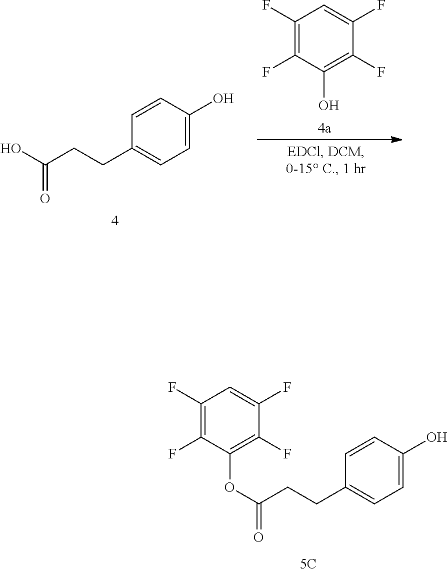 [SMILES: O=C(O)CCc1ccc(O)cc1 ; Oc1c(F)c(F)cc(F)c1F ; O=C(CCc1ccc(O)cc1)Oc1c(F)c(F)cc(F)c1F]

Two batches in parallel. To a solution of EDCI (28.8 g, 150 mmol, 1.00 eq) in DCM (125 mL) is added compound 4a (25.0 g, 150 mmol, 1.00 eq) dropwise at 0° C., then the mixture is added to compound 4 (25.0 g, 150 mmol, 1.00 eq) in DCM (125 mL) at 0° C., then the mixture is stirred at 25° C. for 1 hr. TLC (Petroleum ether:Ethyl acetate=3:1, R_{f}=0.45) showed the reactant is consumed and one new spot is formed. The reaction mixture is diluted with DCM (100 mL) then washed with aq.NaHCO₃ (250 mL*1) and brine (250 mL), dried over Na₂SO₄, filtered and concentrated under reduced pressure to give a residue. The residue is purified by column chromatography (SiO₂, Petroleum ether:Ethyl acetate=100:1 to 3:1), TLC (SiO₂, Petroleum ether:Ethyl acetate=3:1), R_{f}=0.45, then concentrated under reduced pressure to give a residue. Compound 5C (57.0 g, 176 mmol, 58.4% yield, 96.9% purity) is obtained as colorless oil and confirmed ¹HNMR: EW33072-2-P1A, 400 MHz, DMSO b 9.21 (s, 1H), 7.07-7.09 (m, 2H), 6.67-6.70 (m, 2H), 3.02-3.04 (m, 2H), 2.86-2.90 (m, 2H)

### General Procedure for Preparation of Compound 6

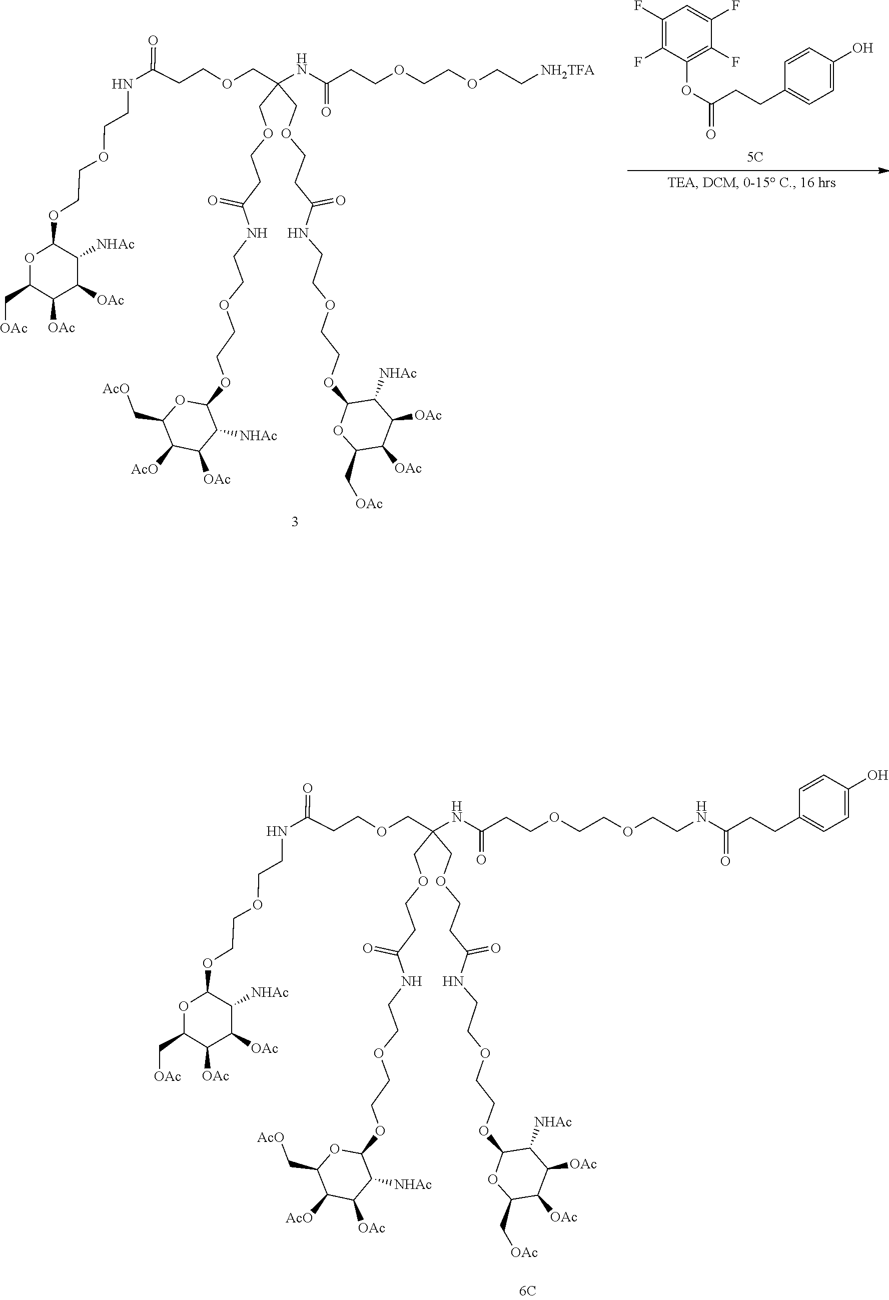

To a mixture of compound 3 (79.0 g, 41.0 mmol, 96.4% purity, 1.00 eq, TFA) and compound 6C (14.2 g, 43.8 mmol, 96.9% purity, 1.07 eq) in DCM (800 mL) is added TEA (16.6 g, 164 mmol, 22.8 mL, 4.00 eq) dropwise at 0° C., the mixture is stirred at 15° C. for 16 hrs. LCMS (EW33072-12-P1B, Rt=0.844 min) showed the desired mass is detected. The reaction mixture is diluted with DCM (400 mL) and washed with aq. NaHCO₃ (400 mL*1) and brine (400 mL*1), then the mixture is diluted with DCM (2.00 L) and washed with 0.7 M Na₂CO₃ (1000 mL*3) and brine (800 mL*3), dried over Na₂SO₄, filtered and concentrated under reduced pressure to give a residue. The residue is used to next step directly without purification. Compound 6 (80.0 g, crude) is obtained as white solid and confirmed via ¹HNMR: EW33072-12-P1A, 400 MHz, MeOD δ 7.02-7.04 (m, 2H), 6.68-6.70 (m, 2H), 5.34-5.35 (s, 3H), 5.07-5.08 (d, J=4.00 Hz, 3H), 4.62-4.64 (d, J=8.00 Hz, 3H), 3.71-4.16 (m, 16H), 3.31-3.70 (m, 44H), 2.80-2.83 (m, 2H), 2.68 (m, 2H), 2.46-2.47 (m, 10H), 2.14 (s, 9H), 2.03 (s, 9H), 1.94-1.95 (d, J=4.00 Hz, 18H).

### General Procedure for Preparation of TriGNal-TRIS-Peg2-Phosph 8c

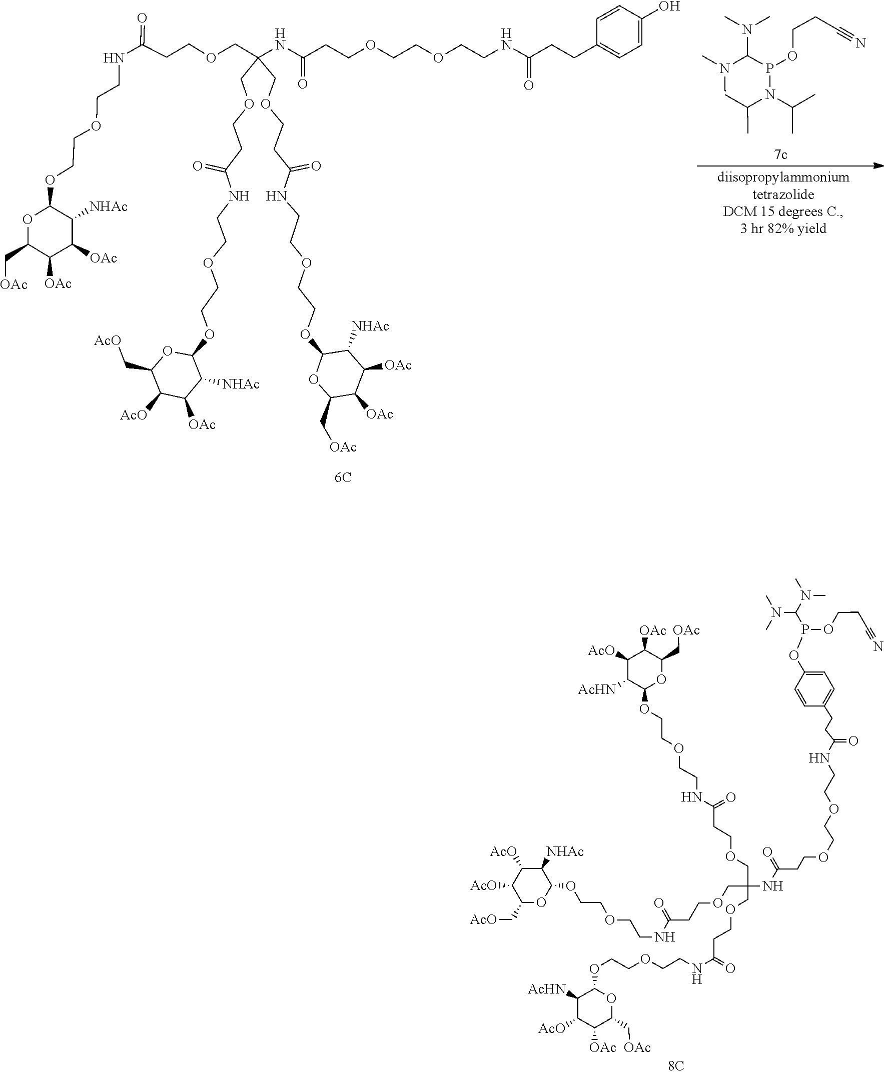

Two batches are synthesized in parallel. To a solution of compound 6C (40.0 g, 21.1 mmol, 1.00 eq in DCM (600 mL) is added diisopropylammonium tetrazolide (3.62 g, 21.1 mmol, 1.00 eq) and compound 7c (6.37 g, 21.1 mmol, 6.71 mL, 1.00 eq) in DCM (8.00 mL) drop-wise, the mixture is stirred at 30° C. for 1 hr, then added compound 7c (3.18 g, 10.6 mmol, 3.35 mL, 0.50 eq) in DCM (8.00 mL) drop-wise, the mixture is stirred at 30° C. for 30 mins, then added compound 7c (3.18 g, 10.6 mmol, 3.35 mL, 0.50 eq) in DCM (8.00 mL) drop-wise, the mixture is stirred at 30° C. for 1.5 hrs. LCMS (EW33072-17-P1C1, Rt=0.921 mi) showed the desired MS+1 is detected. LCMS (EW33072-17-P1C2, Rt=0.919 min) showed the desired MS+1 is detected. Two batches are combined for work-up. The mixture is diluted with DCM (1.20 L), washed with saturated NaHCO₃ aqueous solution (1.60 L*2), 3% DMF in H₂O (1.60 L*2), H₂O (1.60 L*3), brine (1.60 L), dried over Na₂SO₄, filtered and concentrated under reduced pressure to give a residue. The residue is purified by column chromatography (SiO₂, DCM: MeOH:TEA=100:3:2) TLC (SiO₂, DCM: MeOH=10:1, R_{f}=0.45), then concentrated under reduced pressure to give a residue. Compound 8C (76.0 g, 34.8 mmol, 82.5% yield, 96.0% purity) is obtained as white solid and confirmed via ¹HNMR: EW33072-19-P1C, 400 MHz, MeOD δ 7.13-7.15 (d, J=8.50 Hz, 2H), 6.95-6.97 (dd, J=8.38, 1.13 Hz, 2H), 5.34 (d, J=2.88 Hz, 3H), 0.09 (dd, J=11.26, 3.38 Hz, 3H), 4.64 (d, J=8.50 Hz, 3H), 3.99-4.20 (m, 12H), 3.88-3.98 (m, 5H), 3.66-3.83 (m, 20H), 3.51-3.65 (m, 17H), 3.33-3.50 (m, 9H), 2.87 (t, J=7.63 Hz, 2H), 2.76 (t, J=5.94 Hz, 2H), 2.42-2.50 (m, 10H), 2.14 (s, 9H), 2.03 (s, 9H), 1.94-1.95 (d, J=6.13 Hz, 18H), 1.24-1.26 (d, J=6.75 Hz, 6H), 1.18-1.20 (d, J=6.75 Hz, 6H).

### Example 11: Modification Motif 1

An example HGFAC siRNA includes a combination of the following modifications:
Position 9 (from 5′ to 3′) of the sense strand is 2′F.
If position 9 is a pyrimidine then all purines in the Sense Strand are 2′OMe, and 1-5 pyrimidines between positions 5 and 11 are 2′F provided that there are never three 2′F modifications in a row.
If position 9 is a purine then all pyrimidines in the Sense Strand are 2′OMe, and 1-5 purines between positions 5 and 11 are 2′F provided that there are never three 2′F modifications in a row.

Antisense strand odd-numbered positions are 2′OMe and even-numbered positions are a mixture of 2′F, 2′OMe and 2′deoxy.

### Example 12: Modification Motif 2

An example HGFAC siRNA includes a combination of the following modifications:
Position 9 (from 5′ to 3′) of the sense strand is 2′deoxy.
Sense strand positions 5, 7 and 8 are 2′F.
All pyrimidines in positions 10-21 are 2′OMe, and purines are a mixture of 2′OMe and 2′F. Alternatively, all purines in positions 10-21 are 2′OMe and all pyrimidines in positions 10-21 are a mixture of 2′OMe and 2′F.
Antisense strand odd-numbered positions are 2′OMe and even-numbered positions are a mixture of 2′F, 2′OMe and 2′deoxy.

### Example 13. Efficacy Evaluation of ETD02258 Alone and in Combination with Anti-mPD-L1 Against Subcutaneous CT26 WT Murine Colon Carcinoma in Female Balb/c Mice

Four groups (n=20/group) of female Balb/c mice (Envigo) at age 6-7 weeks were utilized for this study. All mice were sorted into study groups based on body weight. The mice were distributed to ensure that the body weight for all groups was within 10% of the overall mean body weight for the study population.

On study Day 0, approximately 500,000 cells of the mouse colorectal adenocarcinoma cell line CT26.WT (ATCC CRL-2638) was injected subcutaneously at a volume of 200 μL into the upper right axilla of each animal. The treatment regimen was started in all groups on study Day 3. Group 1 mice were subcutaneously injected with 100 μL of 1×PBS as a vehicle control on study Days 3, 10, and 17. Group 2 mice were injected subcutaneously with 100 μL of ETD02258 (sense strand, [ETL17]saugcUfuUfGfAfugaaacacgasusu [SEQ ID NO: 4822]; antisense strand, usCfsgUfgUfuUfcAfuCfaAfaGfcAfususu [SEQ ID NO: 4823]) formulated in PBS at a concentration of 2 mg/mL (200 μg total siRNA) on study Days 3, 10, and 17. Group 3 mice were injected intraperitoneally with an anti-mPD-L1 monoclonal antibody (Clone 10F.9G2) reconstituted in Dulbecco's PBS at a concentration of 1 mg/mL on study Days 3,6,10,13,17, and 20. Animals treated with antibody were dosed by full body weight (10 mg/kg). Group 4 mice were treated with combined treatment regimens from Groups 2 and 3. Complete dosing schedules are summarized in Table 18.

All treatments were well tolerated. There were no deaths in the treatment window, and body weight change in the treatment window ranged from of 16.0% to 20.5% body weight gain (Table 19).

In study Days 2,4,7,9,11,14,16,18, 21, and 23, tumor volumes were taken by caliper measurements and calculated using the formula: Tumor burden (mm³)=(L×W²)/2; where L and W are the respective orthogonal tumor length and width measurements (mm). Results are shown in Table 20.

From the tumor volume measurements, additional study endpoints to assess treatment efficacy were determined. Day 21 was chosen for endpoint evaluation because this was the final day all animals on study were measured. 1000 mm³ was selected for Time to Progression (TTP) evaluation because the majority of control animals reached this criterion prior to end of life sampling. Differences in tumor size in the control group compared to the treatment group were determined by comparing the ratio of ΔT/ΔC, where ΔC and ΔT are individual animal endpoints calculated for each animal as follows:
ΔT=Tₜ−T₀ and ΔC=Cₜ−C₀,
where Tₜ and T₀ are the tumor volumes of a treated animal at time t or at the initiation of dosing, respectively. ΔC reflects similar calculations for the control animals. Median ΔT/ΔC is a group endpoint calculated for each day of treatment as:
Median ΔT/ΔC=(ΔT_{med}/ΔC_{med})*100

The results are presented as a percentage. Lower ΔT/ΔC values reflect a smaller overall tumor burden in a group compared to the control group. Time to progression is an individual endpoint and can be used as a surrogate for lifespan or time on study. The selected tumor evaluation size is tumor model and study dependent. TP data is analyzed by Kaplan Meier methods just as traditional lifespan data. The time to progression for an individual animal is the number of days between initiation of treatment and death or the day that the animal reaches a selected evaluation size and may be “>” if animals survive to study termination if the evaluation size is not reached. Results are summarized in Table 21. Single agent therapy with either ETD02258 at 200 ug/animal (Group 2) or anti-mPD-L1 at 10 mg/kg (Group 3) did not result in anti-cancer activity. These treatments resulted in an increase in time to progression of −5 and 0% respectively. Combination therapy with ETD02258 at 200 ug/animal and anti-mPD-L1 at 10 mg/kg (Group 4) outperformed either single agent and resulted in anti-cancer activity. This treatment resulted in an increase in time to progression of >16.6% and significantly less tumor burden at study Day 21.

On either study Day 24 or when animal tumor burden reached a predetermined threshold of 2000 mm³, animals were euthanized and an approximately 30 mg piece of liver excised from each aminal for assessment of target mRNA reduction by RT-qPCR. Liver pieces were placed in 10 v/v RNAlater™ Stabilization Solution (Thermo Fisher, Catalog #AM7020) and stored for at 4° C. until they were shipped to Empirico. The liver samples were processed in homogenization buffer (Maxwell RSC simplyRNA Tissue Kit) using Soft Tissue Homogenizing Kit CK14 (Bertin Instruments, catalog #P000933-LYSKO-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver HGFAC mRNA were assessed in biplexed reactions by RT-qPCR in triplicate using TaqMan assays for mouse HGFAC (ThermoFisher, assay #Mm00469483 ml) and the mouse housekeeping gene PPIA (ThermoFisher, assay #Mm02342430_g1) in PerfeCTa qPCRFastMix Reaction Mix (VWR). The samples were assessed on a QuantStudio™ 6 Pro Real-Time PCR System. The delta-delta Ct method was used to calculate relative amounts of HGFAC mRNA. Group mean relative HGFAC mRNA levels relative to the PBS control Group 1 are shown in Table 22. Treatment with 200 μg of the test article ETD02258 resulted in a decrease in the liver levels of HGFAC mRNA in Groups 2 and 4 compared to untreated control Group 1 and anti-mPD-L1 treated Group 3.

TABLE 18
Dosing Schedule

| | | | | | | Treatment |
|---|---|---|---|---|---|---|
| Group | N | Treatment | Dose | | ROA | Day(s) |
| 1 | 20 | PBS | 100 | μL/injection | SC | 3, 10, 17 |
| 2 | 20 | ETD02258 | 200 | μg/animal | SC | 3, 10, 17 |
| 3 | 20 | anti-mPD-L1 | 10 | mg/kg | IP | 3, 6, 10, |
| | | | | | | 13, 17, 20 |

| | | | | | |
|---|---|---|---|---|---|
| 4 | 20 | anti-mPD-L1 + | 10 mg/kg | IP | 3, 6, 10, |
| | | ETD02258 | 200 μg/animal | SC | 13, 17, 20 |
| | | | | | 3, 10, 17 |

TABLE 19
Mean Body Weights

| | | | Day | | | | | | | | | |
|---|---|---|---|---|---|---|---|---|---|---|---|---|
| | | | 2 | 4 | 7 | 9 | 11 | 14 | 16 | 18 | 21 | 23 |
| Group | n | Treatment | Mean Body Weights (g) | | | | | | | | | |
| 1 | 20 | PBS | 16.8 | 16.7 | 16.9 | 17.2 | 17.4 | 17.9 | 18.5 | 18.8 | 20 | 19.5 |
| 2 | 20 | ETD02258 | 16.9 | 16.8 | 17.0 | 17.2 | 17.2 | 17.7 | 18.5 | 18.9 | 20.2 | 20.3 |
| 3 | 20 | anti-mPD-L1 | 17.3 | 17.2 | 17.4 | 17.6 | 17.7 | 18.2 | 18.6 | 18.9 | 19.7 | 20.0 |
| 4 | 20 | anti-mPD-L1 + | 16.8 | 16.8 | 16.9 | 17.2 | 17.2 | 17.6 | 18 | 18.1 | 18.6 | 19.2 |
| | | ETD02258 | | | | | | | | | | |

TABLE 20
Mean Tumor Volumes

| | | | Day | | | | | | | | | |
|---|---|---|---|---|---|---|---|---|---|---|---|---|
| | | | 2 | 4 | 7 | 9 | 11 | 14 | 16 | 18 | 21 | 23 |
| Group | n | Treatment | Mean Tumor Volumes (mm³) | | | | | | | | | |
| 1 | 20 | PBS | 0 | 10 | 38 | 91 | 176 | 350 | 555 | 786 | 1325 | 1413 |
| 2 | 20 | ETD02258 | 0 | 12 | 41 | 84 | 155 | 286 | 525 | 710 | 1320 | 1589 |
| 3 | 20 | anti-mPD-L1 | 0 | 10 | 42 | 87 | 134 | 255 | 387 | 627 | 981 | 1290 |
| 4 | 20 | anti-mPD-L1 + | 0 | 16 | 60 | 106 | 143 | 224 | 345 | 464 | 559 | 1003 |
| | | ETD02258 | | | | | | | | | | |

TABLE 21
Endpoint Treatment Efficacy Measurements

| | | | | | | Increased |
|---|---|---|---|---|---|---|
| | | | Median Tumor | Tumor Growth | Median | Time to |
| | | | Volume at day | Inhibition | ΔT/ΔC day | Progression |
| Group | n | Treatment | 21 (mm³) | (%) | 21 (%) | (%) |
| 1 | 26 | PBS | 956 | NA | NA | NA |
| 2 | 26 | ETD02258 | 1198 | 0.00 | 125.00 | −5.5 |
| 3 | 26 | anti-mPD-L1 | 926 | 5.00 | 96.00 | 0.00 |
| 4 | 26 | anti-mPD-L1 + | 362 | 62.00 | 37.00 | >16.6 |
| | | ETD02258 | | | | |

TABLE 22
Relative HGFAC mRNA Level in Liver of Mice

| | | | Mean HGFAC mRNA |
|---|---|---|---|
| Group | n | Treatment | (Relative to Group 1) |
| 1 | 20 | PBS | 1.000 |
| 2 | 20 | ETD02258 | 0.071 |
| 3 | 20 | anti-mPD-L1 | 0.570 |
| 4 | 20 | anti-mPD-L1 + ETD02258 | 0.030 |

### Example 14: Protective Variants in HGFAC Result in Altered Levels of Both Cytosolic and Secreted HGFAC

The cDNA of HGFAC protein-coding transcript ENST00000382774.8 was cloned into the pcDNA3.1(+) cDNA expression vector driven by a constitutive CMV promoter. Representative variants from the gene burden test were selected for evaluation. WT (wild-type), C237W (rs201082880), R241X (rs780551152), and L582R (rs138538142) constructs were generated.

Lipid-based plasmid transfection of AR-PE-19 (RPE) cells was used to collect samples to test the functional consequence of HGFAC variants. 5×10⁵ RPE cells were plated in a 6-well dish with DMEM/F12+1000 FBS and grown for 48 hours. Cells were then transfected with 3 μg of each plasmid DNA and 6 μl of Lipofectamine-2000 (a mock-transfected well was included as a control). 48 hours post-transfection, unfiltered culture media was collected for enzyme-linked imuunoassay (ELISA), and cell pellets were collected for RNA and protein isolation. Transfections and analyses were done in biological triplicate.

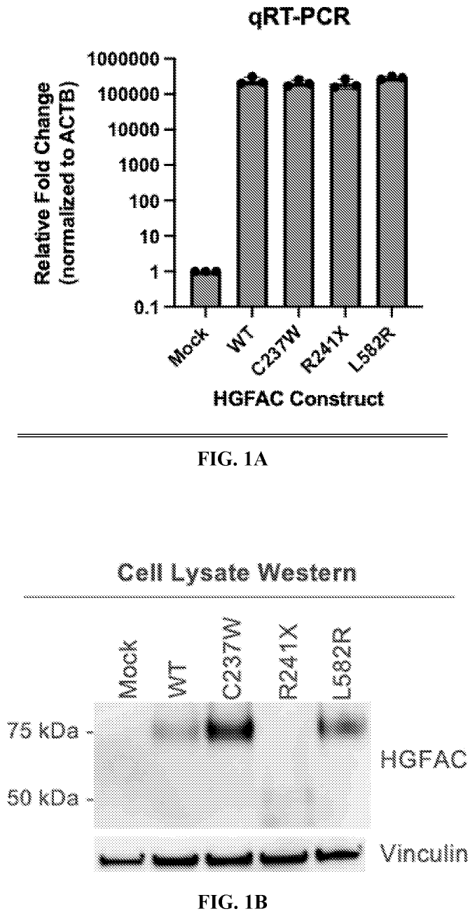

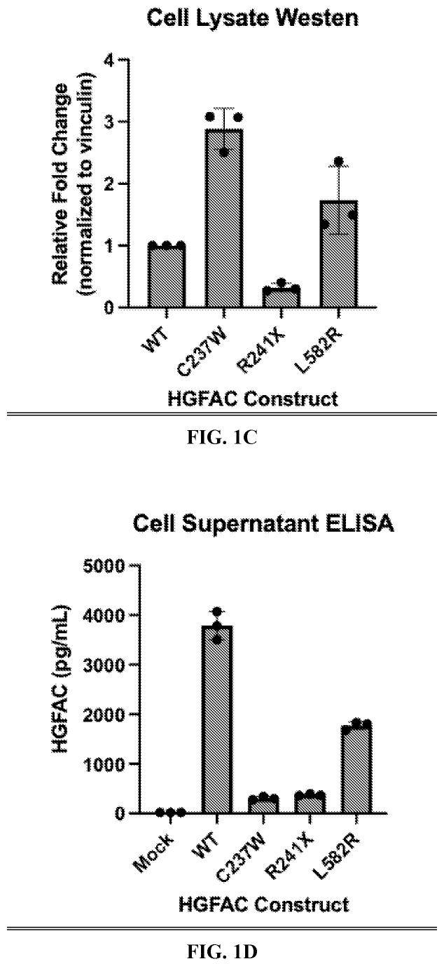

qPCR of poly-A and random-hexamer primed cDNA from isolated RNA of transfected RPE cells indicates robust overexpression of HGFAC mRNA (FIG. 1A). 400 ng of total RNA was used for cDNA synthesis via Bio-Rad's iScript kit. Expression of HGFAC was assessed via SYBR Green and target-specific primers for HGFAC using an ABI QuantStudio5 Pro. Expression was quantified by the ΔΔCt method and normalized to ACTB. Fold change is reported relative to mock-transfected control. No obvious differences were observed in the expression of HGFAC mRNA between WT and and of the variant constructs.

Whole cell protein lysates (WCL) from transfected cells were evaluated for intracellular HGFAC protein by western blot (FIG. 1B). In mock transfected RPE cells, HGFAC was not detectable. In cells transfected with WT, HGFAC was detected by western blot as a band of ˜80 kDa. In cells transfected with the R241X stop gain variant, HGFAC was reduced in molecular weight to ˜50 kDa and its amount was substantially reduced by western blot compared with wild type. In cells transfected with the C237W or L582R missense variants, HGFAC showed elevated levels and this increase was confirmed by quantitative densitometry (FIG. 1C).

ELISAs of culture media from transfected RPE cells demonstrate significant decreases in secreted HGFAC for all three variants relative to WT (FIG. 1D). 100 μL of unfiltered media collected from transfected culture wells was used as input in a commercial sandwich ELISA assay (R&D Systems). Concentrations of secreted HGFAC were imputed from a four-parameter logistic fit of HGFAC standards reconstituted in PBS with 10% FBS.

These data provide experimental verification that representative variants from the cancer-protective HGFAC burden test resulted in either a decrease in total HGFAC levels (R241X) or a decreased capacity to secrete HGFAC protein (C237W and L582R). Additionally, the R241X stop gain variant resulted in a truncated protein.

### Example 15. Testing the Activity of HGFAC siRNA ETD02395 with Alternative Modifications in Mice Transfected with AAV8-TBG-h-HGFAC

The activities of siRNAs with alternative modification patterns of ETD02395, namely ETD02519-ETD02528, were assessed. The siRNAs were attached to the GalNAc ligand ETL17 followed by a phosphorothioate linkage at the 5′ end of the sense strand. The siRNAs used in this Example are included in Table 23, where Nf (e.g. Af, Cf, Gf, Tf, or Uf) is a 2′ fluoro-modified nucleoside, n (e.g. a, c, g, t, or u) is a 2′ O-methyl modified nucleoside, “d” is a 2′ deoxynucleoside dN (e.g. dA, dC, dG, dT, or dU), Nm (e.g. Am, Cm, Gm, Tm, or Um) is a 2′ methoxyethyl modified nucleoside, and “s” is a phosphorothioate linkage. The base sequences for each siRNA, with and without the 3′ UU extension, are shown in Table 24.

Six to eight week old female mice (C57Bl/6) were injected with 5 μL of a recombinant adeno-associated virus 8 (AAV8) vector (1.8×10E13 genome copies/mL) by the retroorbital or tail vein route. The recombinant AAV8 contained the open reading frame and the majority of the 3′UTR of the human HGFAC sequence (GenBank Accession #BC 112190) under the control of the human thyroxine binding globulin promoter in an AAV2 backbone packaged in AAV8 capsid (AAV8-TBG-h-HGFAC).

On Day 16 after infection mice were given a subcutaneous injection of a single 100 μg dose of a GalNAc-conjugated siRNA or PBS as vehicle control. On Day 12 after subcutaneous injection, mice were euthanized and a liver sample from each was collected and placed in RNAlater (ThermoFisher Cat #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver HGFAC mRNA were assessed by RT-qPCR in triplicate on a QuantStudio™ 6 Pro Real-Time PCR System using TaqMan assays for human HGFAC (ThermoFisher, assay #HsOa173526_ml), mouse HGFAC (ThermoFisher, assay #Mm00469483_ml), and the mouse housekeeping gene PPIA (ThermoFisher, assay #Mm02342430_g1) and PerfeCTa® qPCR FastMix®, Low ROX™ (VWR, Catalog #101419-222). Mice with undetectable hHGFAC expression were omitted from further analysis. Data were normalized to the level in animals receiving PBS. The qPCR results using the probe for virally expressed human HGFAC are shown in Table 25, and the qPCR results using the probe for endogenous mouse HGFAC are shown in Table 26.

TABLE 23
Example siRNA Sequences

| | SEQ | Sense Strand Sequence | SEQ | |
|---|---|---|---|---|
| siRNA | ID | (5′-3′) with | ID | Antisense Strand |
| Name | NO: | GalNAc moiety | NO: | Sequence (5′-3′) |
| ETD02395 | 4560 | [ETL17]scuauGfuGfgAfcug | 4497 | usUfsuGfaUfcCfaGfuCfcA |
| | | gaucaaasusu | | fcAfuAfgsusu |
| ETD02519 | 4804 | [ETL17]scuaTmgUfGfGfAf | 4497 | usUfsuGfaUfcCfaGfuCfcA |
| | | cuggmaucaaasusu | | fcAfuAfgsusu |
| ETD02520 | 4805 | [ETL17]scuaTmgUfGfGfAf | 4497 | usUfsuGfaUfcCfaGfuCfcA |
| | | cugmgaucaaasusu | | fcAfuAfgsusu |
| ETD02521 | 4806 | [ETL17]scuauGfuGfgAfcug | 4814 | usUfsugaUfcCfaGfuCfcAfc |
| | | gaucaaasusu | | AfuAfgsusu |
| ETD02522 | 4807 | [ETL17]scuauGfuGfgAfcug | 4815 | usUfsugaUfcCfaGfuccAfc |
| | | gaucaaasusu | | AfuAfgsusu |
| ETD02523 | 4808 | [ETL17]scuauGfuGfgAfcug | 4816 | usUfsugaUfcCfaGfuccAfc |
| | | gaucaaasusu | | Afuagsusu |
| ETD02524 | 4809 | [ETL17]scuauGfuGfgAfcug | 4817 | usUfsugaUfCfcaGfuCfcAfc |
| | | gaucaaasusu | | AfuAfgsusu |
| ETD02525 | 4810 | [ETL17]scuauGfuGfgAfcug | 4818 | usUfsugauCfcaGfuCfcAfc |
| | | gaucaaasusu | | AfuAfgsusu |
| ETD02526 | 4811 | [ETL17]scuauGfuGfgAfcug | 4819 | usUfsugAfuCfcaGfuCfcAfc |
| | | gaucaaasusu | | AfuAfgsusu |
| ETD02527 | 4812 | [ETL17]scuauGfuGfgAfcug | 4820 | usUfsugAfuCfcaGfuccAfc |
| | | gaucaaasusu | | AfuAfgsusu |
| ETD02528 | 4813 | [ETL17]scuauGfuGfgAfcug | 4821 | usUfsugAfuCfcAfguccAfc |
| | | gaucaaasusu | | Afuagsusu |

TABLE 24
Example siRNA BASE Sequences

| | SEQ | Sense Strand | SEQ | Antisense Strand |
|---|---|---|---|---|
| siRNA | ID | Base Sequence | ID | Base Sequence |
| Name | NO: | (5′ to 3′) | NO: | (5′ to 3′) |
| ETD02395 | 4599 | CUAUGUGGACUGGAUCAAAUU | 4654 | UUUGAUCCAGUCCACAUAGUU |
| ETD02519 | 4599 | CUAUGUGGACUGGAUCAAAUU | 4654 | UUUGAUCCAGUCCACAUAGUU |
| ETD02520 | 4599 | CUAUGUGGACUGGAUCAAAUU | 4654 | UUUGAUCCAGUCCACAUAGUU |
| ETD02521 | 4599 | CUAUGUGGACUGGAUCAAAUU | 4654 | UUUGAUCCAGUCCACAUAGUU |
| ETD02522 | 4599 | CUAUGUGGACUGGAUCAAAUU | 4654 | UUUGAUCCAGUCCACAUAGUU |
| ETD02523 | 4599 | CUAUGUGGACUGGAUCAAAUU | 4654 | UUUGAUCCAGUCCACAUAGUU |
| ETD02524 | 4599 | CUAUGUGGACUGGAUCAAAUU | 4654 | UUUGAUCCAGUCCACAUAGUU |
| ETD02525 | 4599 | CUAUGUGGACUGGAUCAAAUU | 4654 | UUUGAUCCAGUCCACAUAGUU |
| ETD02526 | 4599 | CUAUGUGGACUGGAUCAAAUU | 4654 | UUUGAUCCAGUCCACAUAGUU |
| ETD02527 | 4599 | CUAUGUGGACUGGAUCAAAUU | 4654 | UUUGAUCCAGUCCACAUAGUU |
| ETD02528 | 4599 | CUAUGUGGACUGGAUCAAAUU | 4654 | UUUGAUCCAGUCCACAUAGUU |
| | | Sense Strand | | Antisense Strand |
| | SEQ | Base Sequence | SEQ | Base Sequence |
| siRNA | ID | (5′ to 3′), | ID | (5′ to 3′), |
| Name | NO: | without 3′ overhangs | NO: | without 3′ overhangs |
| ETD02395 | 4615 | CUAUGUGGACUGGAUCAAA | 4670 | UUUGAUCCAGUCCACAUAG |
| ETD02519 | 4615 | CUAUGUGGACUGGAUCAAA | 4670 | UUUGAUCCAGUCCACAUAG |
| ETD02520 | 4615 | CUAUGUGGACUGGAUCAAA | 4670 | UUUGAUCCAGUCCACAUAG |
| ETD02521 | 4615 | CUAUGUGGACUGGAUCAAA | 4670 | UUUGAUCCAGUCCACAUAG |
| ETD02522 | 4615 | CUAUGUGGACUGGAUCAAA | 4670 | UUUGAUCCAGUCCACAUAG |
| ETD02523 | 4615 | CUAUGUGGACUGGAUCAAA | 4670 | UUUGAUCCAGUCCACAUAG |
| ETD02524 | 4615 | CUAUGUGGACUGGAUCAAA | 4670 | UUUGAUCCAGUCCACAUAG |
| ETD02525 | 4615 | CUAUGUGGACUGGAUCAAA | 4670 | UUUGAUCCAGUCCACAUAG |
| ETD02526 | 4615 | CUAUGUGGACUGGAUCAAA | 4670 | UUUGAUCCAGUCCACAUAG |
| ETD02527 | 4615 | CUAUGUGGACUGGAUCAAA | 4670 | UUUGAUCCAGUCCACAUAG |
| ETD02528 | 4615 | CUAUGUGGACUGGAUCAAA | 4670 | UUUGAUCCAGUCCACAUAG |

TABLE 25
Relative Human HGFAC mRNA Levels in Livers of AAV8-
TBG-h-HGFAC Mice on Day 12 After siRNA Injection

| | | | Dose | Mean Human HGFAC mRNA |
|---|---|---|---|---|
| Group | n | Treatment | (ug) | (Relative to Group 1, Day 12) |
| 1 | 4 | PBS | | 1.00 |
| 2 | 4 | ETD02395 | 100 | 0.64 |
| 3 | 5 | ETD02519 | 100 | 1.97 |
| 4 | 5 | ETD02520 | 100 | 0.29 |
| 5 | 5 | ETD02521 | 100 | 0.19 |
| 6 | 4 | ETD02522 | 100 | 1.57 |
| 7 | 5 | ETD02523 | 100 | 1.68 |
| 8 | 5 | ETD02524 | 100 | 0.47 |
| 9 | 5 | ETD02525 | 100 | 0.49 |
| 10 | 5 | ETD02526 | 100 | 1.05 |
| 11 | 5 | ETD02527 | 100 | 1.31 |
| 12 | 5 | ETD02528 | 100 | 1.42 |

TABLE 25
Relative Mouse HGFAC mRNA Levels in Livers of AAV8-
TBG-h-HGFAC Mice on Day 12 After siRNA Injection

| | | | Dose | Mean Mouse HGFAC mRNA |
|---|---|---|---|---|
| Group | n | Treatment | (ug) | (Relative to Group 1, Day 12) |
| 1 | 4 | PBS | | 1.00 |
| 2 | 4 | ETD02395 | 100 | 0.14 |
| 3 | 5 | ETD02519 | 100 | 0.15 |
| 4 | 5 | ETD02520 | 100 | 0.12 |
| 5 | 5 | ETD02521 | 100 | 0.08 |
| 6 | 4 | ETD02522 | 100 | 0.13 |
| 7 | 5 | ETD02523 | 100 | 0.21 |
| 8 | 5 | ETD02524 | 100 | 0.12 |
| 9 | 5 | ETD02525 | 100 | 0.16 |
| 10 | 5 | ETD02526 | 100 | 0.13 |
| 11 | 5 | ETD02527 | 100 | 0.10 |
| 12 | 5 | ETD02528 | 100 | 0.12 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and compositions within the scope of these claims and their equivalents be covered thereby.

### IV. Sequence Information

Some embodiments include one or more nucleic acid sequences in the following tables:

Sequence Information

| SEQ ID NO: | Description |
|---|---|
| 1-2051 | HGFAC siRNA sense strand sequences |
| 2052-4102 | HGFAC siRNA antisense strand sequences |
| 4103-4207 | Modified HGFAC siRNA sense strand sequences |
| 4208-4233 | Modified HGFAC siRNA antisense strand sequences |
| 4234-4319 | Modified HGFAC siRNA sense sequences |
| 4320-4354 | Modified HGFAC siRNA antisense sequences |
| 4355-4482 | Modified HGFAC siRNA sense sequences |
| 4483-4499 | Modified HGFAC siRNA antisense sequences |
| 4500-4561 | Modified HGFAC sense sequences |
| 4562-4616 | HGFAC siRNA sense strand sequences |
| 4617-4671 | HGFAC siRNA antisense strand sequences |
| 4672-4711 | Modified HGFAC siRNA sense sequences |
| 4712-4756 | Modified HGFAC siRNA antisense sequences |
| 4757-4779 | HGFAC siRNA sense strand sequences |
| 4780-4802 | HGFAC siRNA antisense strand sequences |
| 4803 | Full-length human HGFAC mRNA sequence (Ensembl |
| | transcript ENST00000382774.8) |
| 4804-4813 | Modified HGFAC siRNA sense sequences |
| 4814-4821 | Modified HGFAC siRNA antisense sequences |

| siRNA | SEQ ID | sense strand | SEQ ID | antisense strand |
|---|---|---|---|---|
| Name | NO: | sequence (5'-3') | NO: | sequence (5'-3') |
| siRNA 1 | 1 | GCCUCCCACUGCCCCUCAG | 2052 | CUGAGGGGCAGUGGGAGGC |
| siRNA 2 | 2 | CCUCCCACUGCCCCUCAGG | 2053 | CCUGAGGGGCAGUGGGAGG |
| siRNA 3 | 3 | CUCCCACUGCCCCUCAGGC | 2054 | GCCUGAGGGGCAGUGGGAG |
| siRNA 4 | 4 | UCCCACUGCCCCUCAGGCC | 2055 | GGCCUGAGGGGCAGUGGGA |
| siRNA 5 | 5 | CCCACUGCCCCUCAGGCCA | 2056 | UGGCCUGAGGGGCAGUGGG |
| siRNA 6 | 6 | CCACUGCCCCUCAGGCCAG | 2057 | CUGGCCUGAGGGGCAGUGG |
| siRNA 7 | 7 | CACUGCCCCUCAGGCCAGC | 2058 | GCUGGCCUGAGGGGCAGUG |
| siRNA 8 | 8 | ACUGCCCCUCAGGCCAGCU | 2059 | AGCUGGCCUGAGGGGCAGU |
| siRNA 9 | 9 | CUGCCCCUCAGGCCAGCUC | 2060 | GAGCUGGCCUGAGGGGCAG |
| siRNA 10 | 10 | UGCCCCUCAGGCCAGCUCA | 2061 | UGAGCUGGCCUGAGGGGCA |
| siRNA 11 | 11 | GCCCCUCAGGCCAGCUCAG | 2062 | CUGAGCUGGCCUGAGGGGC |
| siRNA 12 | 12 | CCCCUCAGGCCAGCUCAGG | 2063 | CCUGAGCUGGCCUGAGGGG |
| siRNA 13 | 13 | CCCUCAGGCCAGCUCAGGA | 2064 | UCCUGAGCUGGCCUGAGGG |
| siRNA 14 | 14 | CCUCAGGCCAGCUCAGGAG | 2065 | CUCCUGAGCUGGCCUGAGG |
| siRNA 15 | 15 | CUCAGGCCAGCUCAGGAGC | 2066 | GCUCCUGAGCUGGCCUGAG |
| siRNA 16 | 16 | UCAGGCCAGCUCAGGAGCC | 2067 | GGCUCCUGAGCUGGCCUGA |
| siRNA 17 | 17 | CAGGCCAGCUCAGGAGCCA | 2068 | UGGCUCCUGAGCUGGCCUG |
| siRNA 18 | 18 | AGGCCAGCUCAGGAGCCAU | 2069 | AUGGCUCCUGAGCUGGCCU |
| siRNA 19 | 19 | GGCCAGCUCAGGAGCCAUG | 2070 | CAUGGCUCCUGAGCUGGCC |
| siRNA 20 | 20 | GCCAGCUCAGGAGCCAUGG | 2071 | CCAUGGCUCCUGAGCUGGC |
| siRNA 21 | 21 | CCAGCUCAGGAGCCAUGGG | 2072 | CCCAUGGCUCCUGAGCUGG |
| siRNA 22 | 22 | CAGCUCAGGAGCCAUGGGG | 2073 | CCCCAUGGCUCCUGAGCUG |
| siRNA 23 | 23 | AGCUCAGGAGCCAUGGGGC | 2074 | GCCCCAUGGCUCCUGAGCU |
| siRNA 24 | 24 | GCUCAGGAGCCAUGGGGCG | 2075 | CGCCCCAUGGCUCCUGAGC |
| siRNA 25 | 25 | CUCAGGAGCCAUGGGGCGC | 2076 | GCGCCCCAUGGCUCCUGAG |
| siRNA 26 | 26 | UCAGGAGCCAUGGGGCGCU | 2077 | AGCGCCCCAUGGCUCCUGA |
| siRNA 27 | 27 | CAGGAGCCAUGGGGCGCUG | 2078 | CAGCGCCCCAUGGCUCCUG |
| siRNA 28 | 28 | AGGAGCCAUGGGGCGCUGG | 2079 | CCAGCGCCCCAUGGCUCCU |
| siRNA 29 | 29 | GGAGCCAUGGGGCGCUGGG | 2080 | CCCAGCGCCCCAUGGCUCC |
| siRNA 30 | 30 | GAGCCAUGGGGCGCUGGGC | 2081 | GCCCAGCGCCCCAUGGCUC |
| siRNA 31 | 31 | AGCCAUGGGGCGCUGGGCC | 2082 | GGCCCAGCGCCCCAUGGCU |
| siRNA 32 | 32 | GCCAUGGGGCGCUGGGCCU | 2083 | AGGCCCAGCGCCCCAUGGC |
| siRNA 33 | 33 | CCAUGGGGCGCUGGGCCUG | 2084 | CAGGCCCAGCGCCCCAUGG |
| siRNA 34 | 34 | CAUGGGGCGCUGGGCCUGG | 2085 | CCAGGCCCAGCGCCCCAUG |
| siRNA 35 | 35 | AUGGGGCGCUGGGCCUGGG | 2086 | CCCAGGCCCAGCGCCCCAU |
| siRNA 36 | 36 | UGGGGCGCUGGGCCUGGGU | 2087 | ACCCAGGCCCAGCGCCCCA |
| siRNA 37 | 37 | GGGGCGCUGGGCCUGGGUC | 2088 | GACCCAGGCCCAGCGCCCC |
| siRNA 38 | 38 | GGGCGCUGGGCCUGGGUCC | 2089 | GGACCCAGGCCCAGCGCCC |
| siRNA 39 | 39 | GGCGCUGGGCCUGGGUCCC | 2090 | GGGACCCAGGCCCAGCGCC |
| siRNA 40 | 40 | GCGCUGGGCCUGGGUCCCC | 2091 | GGGGACCCAGGCCCAGCGC |
| siRNA 41 | 41 | CGCUGGGCCUGGGUCCCCA | 2092 | UGGGGACCCAGGCCCAGCG |
| siRNA 42 | 42 | GCUGGGCCUGGGUCCCCAG | 2093 | CUGGGGACCCAGGCCCAGC |
| siRNA 43 | 43 | CUGGGCCUGGGUCCCCAGC | 2094 | GCUGGGGACCCAGGCCCAG |
| siRNA 44 | 44 | UGGGCCUGGGUCCCCAGCC | 2095 | GGCUGGGGACCCAGGCCCA |
| siRNA 45 | 45 | GGGCCUGGGUCCCCAGCCC | 2096 | GGGCUGGGGACCCAGGCCC |
| siRNA 46 | 46 | GGCCUGGGUCCCCAGCCCC | 2097 | GGGGCUGGGGACCCAGGCC |
| siRNA 47 | 47 | GCCUGGGUCCCCAGCCCCU | 2098 | AGGGGCUGGGGACCCAGGC |
| siRNA 48 | 48 | CCUGGGUCCCCAGCCCCUG | 2099 | CAGGGGCUGGGGACCCAGG |
| siRNA 49 | 49 | CUGGGUCCCCAGCCCCUGG | 2100 | CCAGGGGCUGGGGACCCAG |
| siRNA 50 | 50 | UGGGUCCCCAGCCCCUGGC | 2101 | GCCAGGGGCUGGGGACCCA |
| siRNA 51 | 51 | GGGUCCCCAGCCCCUGGCC | 2102 | GGCCAGGGGCUGGGGACCC |
| siRNA 52 | 52 | GGUCCCCAGCCCCUGGCCC | 2103 | GGGCCAGGGGCUGGGGACC |
| siRNA 53 | 53 | GUCCCCAGCCCCUGGCCCC | 2104 | GGGGCCAGGGGCUGGGGAC |
| siRNA 54 | 54 | UCCCCAGCCCCUGGCCCCC | 2105 | GGGGGCCAGGGGCUGGGGA |
| siRNA 55 | 55 | CCCCAGCCCCUGGCCCCCA | 2106 | UGGGGGCCAGGGGCUGGGG |
| siRNA 56 | 56 | CCCAGCCCCUGGCCCCCAC | 2107 | GUGGGGGCCAGGGGCUGGG |
| siRNA 57 | 57 | CCAGCCCCUGGCCCCCACC | 2108 | GGUGGGGGCCAGGGGCUGG |
| siRNA 58 | 58 | CAGCCCCUGGCCCCCACCG | 2109 | CGGUGGGGGCCAGGGGCUG |
| siRNA 59 | 59 | AGCCCCUGGCCCCCACCGG | 2110 | CCGGUGGGGGCCAGGGGCU |
| siRNA 60 | 60 | GCCCCUGGCCCCCACCGGG | 2111 | CCCGGUGGGGGCCAGGGGC |
| siRNA 61 | 61 | CCCCUGGCCCCCACCGGGG | 2112 | CCCCGGUGGGGGCCAGGGG |
| siRNA 62 | 62 | CCCUGGCCCCCACCGGGGC | 2113 | GCCCCGGUGGGGGCCAGGG |
| siRNA 63 | 63 | CCUGGCCCCCACCGGGGCU | 2114 | AGCCCCGGUGGGGGCCAGG |
| siRNA 64 | 64 | CUGGCCCCCACCGGGGCUG | 2115 | CAGCCCCGGUGGGGGCCAG |
| siRNA 65 | 65 | UGGCCCCCACCGGGGCUGG | 2116 | CCAGCCCCGGUGGGGGCCA |
| siRNA 66 | 66 | GGCCCCCACCGGGGCUGGG | 2117 | CCCAGCCCCGGUGGGGGCC |
| siRNA 67 | 67 | GCCCCCACCGGGGCUGGGC | 2118 | GCCCAGCCCCGGUGGGGGC |
| siRNA 68 | 68 | CCCCCACCGGGGCUGGGCC | 2119 | GGCCCAGCCCCGGUGGGGG |
| siRNA 69 | 69 | CCCCACCGGGGCUGGGCCC | 2120 | GGGCCCAGCCCCGGUGGGG |
| siRNA 70 | 70 | CCCACCGGGGCUGGGCCCC | 2121 | GGGGCCCAGCCCCGGUGGG |
| siRNA 71 | 71 | CCACCGGGGCUGGGCCCCU | 2122 | AGGGGCCCAGCCCCGGUGG |
| siRNA 72 | 72 | CACCGGGGCUGGGCCCCUU | 2123 | AAGGGGCCCAGCCCCGGUG |
| siRNA 73 | 73 | ACCGGGGCUGGGCCCCUUC | 2124 | GAAGGGGCCCAGCCCCGGU |
| siRNA 74 | 74 | CCGGGGCUGGGCCCCUUCC | 2125 | GGAAGGGGCCCAGCCCCGG |
| siRNA 75 | 75 | CGGGGCUGGGCCCCUUCCU | 2126 | AGGAAGGGGCCCAGCCCCG |
| siRNA 76 | 76 | GGGGCUGGGCCCCUUCCUC | 2127 | GAGGAAGGGGCCCAGCCCC |
| siRNA 77 | 77 | GGGCUGGGCCCCUUCCUCC | 2128 | GGAGGAAGGGGCCCAGCCC |
| siRNA 78 | 78 | GGCUGGGCCCCUUCCUCCU | 2129 | AGGAGGAAGGGGCCCAGCC |
| siRNA 79 | 79 | GCUGGGCCCCUUCCUCCUC | 2130 | GAGGAGGAAGGGGCCCAGC |
| siRNA 80 | 80 | CUGGGCCCCUUCCUCCUCC | 2131 | GGAGGAGGAAGGGGCCCAG |
| siRNA 81 | 81 | UGGGCCCCUUCCUCCUCCU | 2132 | AGGAGGAGGAAGGGGCCCA |
| siRNA 82 | 82 | GGGCCCCUUCCUCCUCCUC | 2133 | GAGGAGGAGGAAGGGGCCC |
| siRNA 83 | 83 | GGCCCCUUCCUCCUCCUCC | 2134 | GGAGGAGGAGGAAGGGGCC |
| siRNA 84 | 84 | GCCCCUUCCUCCUCCUCCU | 2135 | AGGAGGAGGAGGAAGGGGC |
| siRNA 85 | 85 | CCCCUUCCUCCUCCUCCUC | 2136 | GAGGAGGAGGAGGAAGGGG |
| siRNA 86 | 86 | CCCUUCCUCCUCCUCCUCC | 2137 | GGAGGAGGAGGAGGAAGGG |
| siRNA 87 | 87 | CCUUCCUCCUCCUCCUCCU | 2138 | AGGAGGAGGAGGAGGAAGG |
| siRNA 88 | 88 | CUUCCUCCUCCUCCUCCUG | 2139 | CAGGAGGAGGAGGAGGAAG |
| siRNA 89 | 89 | UUCCUCCUCCUCCUCCUGC | 2140 | GCAGGAGGAGGAGGAGGAA |
| siRNA 90 | 90 | UCCUCCUCCUCCUCCUGCU | 2141 | AGCAGGAGGAGGAGGAGGA |
| siRNA 91 | 91 | CCUCCUCCUCCUCCUGCUG | 2142 | CAGCAGGAGGAGGAGGAGG |
| siRNA 92 | 92 | CUCCUCCUCCUCCUGCUGC | 2143 | GCAGCAGGAGGAGGAGGAG |
| siRNA 93 | 93 | UCCUCCUCCUCCUGCUGCU | 2144 | AGCAGCAGGAGGAGGAGGA |
| siRNA 94 | 94 | CCUCCUCCUCCUGCUGCUG | 2145 | CAGCAGCAGGAGGAGGAGG |
| siRNA 95 | 95 | CUCCUCCUCCUGCUGCUGC | 2146 | GCAGCAGCAGGAGGAGGAG |
| siRNA 96 | 96 | UCCUCCUCCUGCUGCUGCU | 2147 | AGCAGCAGCAGGAGGAGGA |
| siRNA 97 | 97 | CCUCCUCCUGCUGCUGCUG | 2148 | CAGCAGCAGCAGGAGGAGG |
| siRNA 98 | 98 | CUCCUCCUGCUGCUGCUGC | 2149 | GCAGCAGCAGCAGGAGGAG |
| siRNA 99 | 99 | UCCUCCUGCUGCUGCUGCU | 2150 | AGCAGCAGCAGCAGGAGGA |
| siRNA 100 | 100 | CCUCCUGCUGCUGCUGCUG | 2151 | CAGCAGCAGCAGCAGGAGG |
| siRNA 101 | 101 | CUCCUGCUGCUGCUGCUGC | 2152 | GCAGCAGCAGCAGCAGGAG |
| siRNA 102 | 102 | UCCUGCUGCUGCUGCUGCU | 2153 | AGCAGCAGCAGCAGCAGGA |
| siRNA 103 | 103 | CCUGCUGCUGCUGCUGCUG | 2154 | CAGCAGCAGCAGCAGCAGG |
| siRNA 104 | 104 | CUGCUGCUGCUGCUGCUGC | 2155 | GCAGCAGCAGCAGCAGCAG |
| siRNA 105 | 105 | UGCUGCUGCUGCUGCUGCC | 2156 | GGCAGCAGCAGCAGCAGCA |
| siRNA 106 | 106 | GCUGCUGCUGCUGCUGCCA | 2157 | UGGCAGCAGCAGCAGCAGC |
| siRNA 107 | 107 | CUGCUGCUGCUGCUGCCAC | 2158 | GUGGCAGCAGCAGCAGCAG |
| siRNA 108 | 108 | UGCUGCUGCUGCUGCCACG | 2159 | CGUGGCAGCAGCAGCAGCA |
| siRNA 109 | 109 | GCUGCUGCUGCUGCCACGG | 2160 | CCGUGGCAGCAGCAGCAGC |
| siRNA 110 | 110 | CUGCUGCUGCUGCCACGGG | 2161 | CCCGUGGCAGCAGCAGCAG |
| siRNA 111 | 111 | UGCUGCUGCUGCCACGGGG | 2162 | CCCCGUGGCAGCAGCAGCA |
| siRNA 112 | 112 | GCUGCUGCUGCCACGGGGG | 2163 | CCCCCGUGGCAGCAGCAGC |
| siRNA 113 | 113 | CUGCUGCUGCCACGGGGGU | 2164 | ACCCCCGUGGCAGCAGCAG |
| siRNA 114 | 114 | UGCUGCUGCCACGGGGGUU | 2165 | AACCCCCGUGGCAGCAGCA |
| siRNA 115 | 115 | GCUGCUGCCACGGGGGUUC | 2166 | GAACCCCCGUGGCAGCAGC |
| siRNA 116 | 116 | CUGCUGCCACGGGGGUUCC | 2167 | GGAACCCCCGUGGCAGCAG |
| siRNA 117 | 117 | UGCUGCCACGGGGGUUCCA | 2168 | UGGAACCCCCGUGGCAGCA |
| siRNA 118 | 118 | GCUGCCACGGGGGUUCCAG | 2169 | CUGGAACCCCCGUGGCAGC |
| siRNA 119 | 119 | CUGCCACGGGGGUUCCAGC | 2170 | GCUGGAACCCCCGUGGCAG |
| siRNA 120 | 120 | UGCCACGGGGGUUCCAGCC | 2171 | GGCUGGAACCCCCGUGGCA |
| siRNA 121 | 121 | GCCACGGGGGUUCCAGCCC | 2172 | GGGCUGGAACCCCCGUGGC |
| siRNA 122 | 122 | CCACGGGGGUUCCAGCCCC | 2173 | GGGGCUGGAACCCCCGUGG |
| siRNA 123 | 123 | CACGGGGGUUCCAGCCCCA | 2174 | UGGGGCUGGAACCCCCGUG |
| siRNA 124 | 124 | ACGGGGGUUCCAGCCCCAG | 2175 | CUGGGGCUGGAACCCCCGU |
| siRNA 125 | 125 | CGGGGGUUCCAGCCCCAGC | 2176 | GCUGGGGCUGGAACCCCCG |
| siRNA 126 | 126 | GGGGGUUCCAGCCCCAGCC | 2177 | GGCUGGGGCUGGAACCCCC |
| siRNA 127 | 127 | GGGGUUCCAGCCCCAGCCU | 2178 | AGGCUGGGGCUGGAACCCC |
| siRNA 128 | 128 | GGGUUCCAGCCCCAGCCUG | 2179 | CAGGCUGGGGCUGGAACCC |
| siRNA 129 | 129 | GGUUCCAGCCCCAGCCUGG | 2180 | CCAGGCUGGGGCUGGAACC |
| siRNA 130 | 130 | GUUCCAGCCCCAGCCUGGC | 2181 | GCCAGGCUGGGGCUGGAAC |
| siRNA 131 | 131 | UUCCAGCCCCAGCCUGGCG | 2182 | CGCCAGGCUGGGGCUGGAA |
| siRNA 132 | 132 | UCCAGCCCCAGCCUGGCGG | 2183 | CCGCCAGGCUGGGGCUGGA |
| siRNA 133 | 133 | CCAGCCCCAGCCUGGCGGG | 2184 | CCCGCCAGGCUGGGGCUGG |
| siRNA 134 | 134 | CAGCCCCAGCCUGGCGGGA | 2185 | UCCCGCCAGGCUGGGGCUG |
| siRNA 135 | 135 | AGCCCCAGCCUGGCGGGAA | 2186 | UUCCCGCCAGGCUGGGGCU |
| siRNA 136 | 136 | GCCCCAGCCUGGCGGGAAC | 2187 | GUUCCCGCCAGGCUGGGGC |
| siRNA 137 | 137 | CCCCAGCCUGGCGGGAACC | 2188 | GGUUCCCGCCAGGCUGGGG |
| siRNA 138 | 138 | CCCAGCCUGGCGGGAACCG | 2189 | CGGUUCCCGCCAGGCUGGG |
| siRNA 139 | 139 | CCAGCCUGGCGGGAACCGU | 2190 | ACGGUUCCCGCCAGGCUGG |
| siRNA 140 | 140 | CAGCCUGGCGGGAACCGUA | 2191 | UACGGUUCCCGCCAGGCUG |
| siRNA 141 | 141 | AGCCUGGCGGGAACCGUAC | 2192 | GUACGGUUCCCGCCAGGCU |
| siRNA 142 | 142 | GCCUGGCGGGAACCGUACG | 2193 | CGUACGGUUCCCGCCAGGC |
| siRNA 143 | 143 | CCUGGCGGGAACCGUACGG | 2194 | CCGUACGGUUCCCGCCAGG |
| siRNA 144 | 144 | CUGGCGGGAACCGUACGGA | 2195 | UCCGUACGGUUCCCGCCAG |
| siRNA 145 | 145 | UGGCGGGAACCGUACGGAG | 2196 | CUCCGUACGGUUCCCGCCA |
| siRNA 146 | 146 | GGCGGGAACCGUACGGAGU | 2197 | ACUCCGUACGGUUCCCGCC |
| siRNA 147 | 147 | GCGGGAACCGUACGGAGUC | 2198 | GACUCCGUACGGUUCCCGC |
| siRNA 148 | 148 | CGGGAACCGUACGGAGUCC | 2199 | GGACUCCGUACGGUUCCCG |
| siRNA 149 | 149 | GGGAACCGUACGGAGUCCC | 2200 | GGGACUCCGUACGGUUCCC |
| siRNA 150 | 150 | GGAACCGUACGGAGUCCCC | 2201 | GGGGACUCCGUACGGUUCC |
| siRNA 151 | 151 | GAACCGUACGGAGUCCCCA | 2202 | UGGGGACUCCGUACGGUUC |
| siRNA 152 | 152 | AACCGUACGGAGUCCCCAG | 2203 | CUGGGGACUCCGUACGGUU |
| siRNA 153 | 153 | ACCGUACGGAGUCCCCAGA | 2204 | UCUGGGGACUCCGUACGGU |
| siRNA 154 | 154 | CCGUACGGAGUCCCCAGAA | 2205 | UUCUGGGGACUCCGUACGG |
| siRNA 155 | 155 | CGUACGGAGUCCCCAGAAC | 2206 | GUUCUGGGGACUCCGUACG |
| siRNA 156 | 156 | GUACGGAGUCCCCAGAACC | 2207 | GGUUCUGGGGACUCCGUAC |
| siRNA 157 | 157 | UACGGAGUCCCCAGAACCU | 2208 | AGGUUCUGGGGACUCCGUA |
| siRNA 158 | 158 | ACGGAGUCCCCAGAACCUA | 2209 | UAGGUUCUGGGGACUCCGU |
| siRNA 159 | 159 | CGGAGUCCCCAGAACCUAA | 2210 | UUAGGUUCUGGGGACUCCG |
| siRNA 160 | 160 | GGAGUCCCCAGAACCUAAU | 2211 | AUUAGGUUCUGGGGACUCC |
| siRNA 161 | 161 | GAGUCCCCAGAACCUAAUG | 2212 | CAUUAGGUUCUGGGGACUC |
| siRNA 162 | 162 | AGUCCCCAGAACCUAAUGC | 2213 | GCAUUAGGUUCUGGGGACU |
| siRNA 163 | 163 | GUCCCCAGAACCUAAUGCC | 2214 | GGCAUUAGGUUCUGGGGAC |
| siRNA 164 | 164 | UCCCCAGAACCUAAUGCCA | 2215 | UGGCAUUAGGUUCUGGGGA |
| siRNA 165 | 165 | CCCCAGAACCUAAUGCCAC | 2216 | GUGGCAUUAGGUUCUGGGG |
| siRNA 166 | 166 | CCCAGAACCUAAUGCCACA | 2217 | UGUGGCAUUAGGUUCUGGG |
| siRNA 167 | 167 | CCAGAACCUAAUGCCACAG | 2218 | CUGUGGCAUUAGGUUCUGG |
| siRNA 168 | 168 | CAGAACCUAAUGCCACAGC | 2219 | GCUGUGGCAUUAGGUUCUG |
| siRNA 169 | 169 | AGAACCUAAUGCCACAGCG | 2220 | CGCUGUGGCAUUAGGUUCU |
| siRNA 170 | 170 | GAACCUAAUGCCACAGCGA | 2221 | UCGCUGUGGCAUUAGGUUC |
| siRNA 171 | 171 | AACCUAAUGCCACAGCGAC | 2222 | GUCGCUGUGGCAUUAGGUU |
| siRNA 172 | 172 | ACCUAAUGCCACAGCGACC | 2223 | GGUCGCUGUGGCAUUAGGU |
| siRNA 173 | 173 | CCUAAUGCCACAGCGACCC | 2224 | GGGUCGCUGUGGCAUUAGG |
| siRNA 174 | 174 | CUAAUGCCACAGCGACCCC | 2225 | GGGGUCGCUGUGGCAUUAG |
| siRNA 175 | 175 | UAAUGCCACAGCGACCCCU | 2226 | AGGGGUCGCUGUGGCAUUA |
| siRNA 176 | 176 | AAUGCCACAGCGACCCCUG | 2227 | CAGGGGUCGCUGUGGCAUU |
| siRNA 177 | 177 | AUGCCACAGCGACCCCUGC | 2228 | GCAGGGGUCGCUGUGGCAU |
| siRNA 178 | 178 | UGCCACAGCGACCCCUGCG | 2229 | CGCAGGGGUCGCUGUGGCA |
| siRNA 179 | 179 | GCCACAGCGACCCCUGCGA | 2230 | UCGCAGGGGUCGCUGUGGC |
| siRNA 180 | 180 | CCACAGCGACCCCUGCGAU | 2231 | AUCGCAGGGGUCGCUGUGG |
| siRNA 181 | 181 | CACAGCGACCCCUGCGAUC | 2232 | GAUCGCAGGGGUCGCUGUG |
| siRNA 182 | 182 | ACAGCGACCCCUGCGAUCC | 2233 | GGAUCGCAGGGGUCGCUGU |
| siRNA 183 | 183 | CAGCGACCCCUGCGAUCCC | 2234 | GGGAUCGCAGGGGUCGCUG |
| siRNA 184 | 184 | AGCGACCCCUGCGAUCCCC | 2235 | GGGGAUCGCAGGGGUCGCU |
| siRNA 185 | 185 | GCGACCCCUGCGAUCCCCA | 2236 | UGGGGAUCGCAGGGGUCGC |
| siRNA 186 | 186 | CGACCCCUGCGAUCCCCAC | 2237 | GUGGGGAUCGCAGGGGUCG |
| siRNA 187 | 187 | GACCCCUGCGAUCCCCACU | 2238 | AGUGGGGAUCGCAGGGGUC |
| siRNA 188 | 188 | ACCCCUGCGAUCCCCACUA | 2239 | UAGUGGGGAUCGCAGGGGU |
| siRNA 189 | 189 | CCCCUGCGAUCCCCACUAU | 2240 | AUAGUGGGGAUCGCAGGGG |
| siRNA 190 | 190 | CCCUGCGAUCCCCACUAUC | 2241 | GAUAGUGGGGAUCGCAGGG |
| siRNA 191 | 191 | CCUGCGAUCCCCACUAUCC | 2242 | GGAUAGUGGGGAUCGCAGG |
| siRNA 192 | 192 | CUGCGAUCCCCACUAUCCU | 2243 | AGGAUAGUGGGGAUCGCAG |
| siRNA 193 | 193 | UGCGAUCCCCACUAUCCUG | 2244 | CAGGAUAGUGGGGAUCGCA |
| siRNA 194 | 194 | GCGAUCCCCACUAUCCUGG | 2245 | CCAGGAUAGUGGGGAUCGC |
| siRNA 195 | 195 | CGAUCCCCACUAUCCUGGU | 2246 | ACCAGGAUAGUGGGGAUCG |
| siRNA 196 | 196 | GAUCCCCACUAUCCUGGUG | 2247 | CACCAGGAUAGUGGGGAUC |
| siRNA 197 | 197 | AUCCCCACUAUCCUGGUGA | 2248 | UCACCAGGAUAGUGGGGAU |
| siRNA 198 | 198 | UCCCCACUAUCCUGGUGAC | 2249 | GUCACCAGGAUAGUGGGGA |
| siRNA 199 | 199 | CCCCACUAUCCUGGUGACC | 2250 | GGUCACCAGGAUAGUGGGG |
| siRNA 200 | 200 | CCCACUAUCCUGGUGACCU | 2251 | AGGUCACCAGGAUAGUGGG |
| siRNA 201 | 201 | CCACUAUCCUGGUGACCUC | 2252 | GAGGUCACCAGGAUAGUGG |
| siRNA 202 | 202 | CACUAUCCUGGUGACCUCU | 2253 | AGAGGUCACCAGGAUAGUG |
| siRNA 203 | 203 | ACUAUCCUGGUGACCUCUG | 2254 | CAGAGGUCACCAGGAUAGU |
| siRNA 204 | 204 | CUAUCCUGGUGACCUCUGU | 2255 | ACAGAGGUCACCAGGAUAG |
| siRNA 205 | 205 | UAUCCUGGUGACCUCUGUG | 2256 | CACAGAGGUCACCAGGAUA |
| siRNA 206 | 206 | AUCCUGGUGACCUCUGUGA | 2257 | UCACAGAGGUCACCAGGAU |
| siRNA 207 | 207 | UCCUGGUGACCUCUGUGAC | 2258 | GUCACAGAGGUCACCAGGA |
| siRNA 208 | 208 | CCUGGUGACCUCUGUGACC | 2259 | GGUCACAGAGGUCACCAGG |
| siRNA 209 | 209 | CUGGUGACCUCUGUGACCU | 2260 | AGGUCACAGAGGUCACCAG |
| siRNA 210 | 210 | UGGUGACCUCUGUGACCUC | 2261 | GAGGUCACAGAGGUCACCA |
| siRNA 211 | 211 | GGUGACCUCUGUGACCUCU | 2262 | AGAGGUCACAGAGGUCACC |
| siRNA 212 | 212 | GUGACCUCUGUGACCUCUG | 2263 | CAGAGGUCACAGAGGUCAC |
| siRNA 213 | 213 | UGACCUCUGUGACCUCUGA | 2264 | UCAGAGGUCACAGAGGUCA |
| siRNA 214 | 214 | GACCUCUGUGACCUCUGAG | 2265 | CUCAGAGGUCACAGAGGUC |
| siRNA 215 | 215 | ACCUCUGUGACCUCUGAGA | 2266 | UCUCAGAGGUCACAGAGGU |
| siRNA 216 | 216 | CCUCUGUGACCUCUGAGAC | 2267 | GUCUCAGAGGUCACAGAGG |
| siRNA 217 | 217 | CUCUGUGACCUCUGAGACC | 2268 | GGUCUCAGAGGUCACAGAG |
| siRNA 218 | 218 | UCUGUGACCUCUGAGACCC | 2269 | GGGUCUCAGAGGUCACAGA |
| siRNA 219 | 219 | CUGUGACCUCUGAGACCCC | 2270 | GGGGUCUCAGAGGUCACAG |
| siRNA 220 | 220 | UGUGACCUCUGAGACCCCA | 2271 | UGGGGUCUCAGAGGUCACA |
| siRNA 221 | 221 | GUGACCUCUGAGACCCCAG | 2272 | CUGGGGUCUCAGAGGUCAC |
| siRNA 222 | 222 | UGACCUCUGAGACCCCAGC | 2273 | GCUGGGGUCUCAGAGGUCA |
| siRNA 223 | 223 | GACCUCUGAGACCCCAGCA | 2274 | UGCUGGGGUCUCAGAGGUC |
| siRNA 224 | 224 | ACCUCUGAGACCCCAGCAA | 2275 | UUGCUGGGGUCUCAGAGGU |
| siRNA 225 | 225 | CCUCUGAGACCCCAGCAAC | 2276 | GUUGCUGGGGUCUCAGAGG |
| siRNA 226 | 226 | CUCUGAGACCCCAGCAACA | 2277 | UGUUGCUGGGGUCUCAGAG |
| siRNA 227 | 227 | UCUGAGACCCCAGCAACAA | 2278 | UUGUUGCUGGGGUCUCAGA |
| siRNA 228 | 228 | CUGAGACCCCAGCAACAAG | 2279 | CUUGUUGCUGGGGUCUCAG |
| siRNA 229 | 229 | UGAGACCCCAGCAACAAGU | 2280 | ACUUGUUGCUGGGGUCUCA |
| siRNA 230 | 230 | GAGACCCCAGCAACAAGUG | 2281 | CACUUGUUGCUGGGGUCUC |
| siRNA 231 | 231 | AGACCCCAGCAACAAGUGC | 2282 | GCACUUGUUGCUGGGGUCU |
| siRNA 232 | 232 | GACCCCAGCAACAAGUGCU | 2283 | AGCACUUGUUGCUGGGGUC |
| siRNA 233 | 233 | ACCCCAGCAACAAGUGCUC | 2284 | GAGCACUUGUUGCUGGGGU |
| siRNA 234 | 234 | CCCCAGCAACAAGUGCUCC | 2285 | GGAGCACUUGUUGCUGGGG |
| siRNA 235 | 235 | CCCAGCAACAAGUGCUCCA | 2286 | UGGAGCACUUGUUGCUGGG |
| siRNA 236 | 236 | CCAGCAACAAGUGCUCCAG | 2287 | CUGGAGCACUUGUUGCUGG |
| siRNA 237 | 237 | CAGCAACAAGUGCUCCAGA | 2288 | UCUGGAGCACUUGUUGCUG |
| siRNA 238 | 238 | AGCAACAAGUGCUCCAGAG | 2289 | CUCUGGAGCACUUGUUGCU |
| SiRNA 239 | 239 | GCAACAAGUGCUCCAGAGG | 2290 | CCUCUGGAGCACUUGUUGC |
| siRNA 240 | 240 | CAACAAGUGCUCCAGAGGC | 2291 | GCCUCUGGAGCACUUGUUG |
| siRNA 241 | 241 | AACAAGUGCUCCAGAGGCA | 2292 | UGCCUCUGGAGCACUUGUU |
| siRNA 242 | 242 | ACAAGUGCUCCAGAGGCAG | 2293 | CUGCCUCUGGAGCACUUGU |
| siRNA 243 | 243 | CAAGUGCUCCAGAGGCAGA | 2294 | UCUGCCUCUGGAGCACUUG |
| siRNA 244 | 244 | AAGUGCUCCAGAGGCAGAG | 2295 | CUCUGCCUCUGGAGCACUU |
| siRNA 245 | 245 | AGUGCUCCAGAGGCAGAGG | 2296 | CCUCUGCCUCUGGAGCACU |
| siRNA 246 | 246 | GUGCUCCAGAGGCAGAGGG | 2297 | CCCUCUGCCUCUGGAGCAC |
| siRNA 247 | 247 | UGCUCCAGAGGCAGAGGGA | 2298 | UCCCUCUGCCUCUGGAGCA |
| siRNA 248 | 248 | GCUCCAGAGGCAGAGGGAC | 2299 | GUCCCUCUGCCUCUGGAGC |
| siRNA 249 | 249 | CUCCAGAGGCAGAGGGACC | 2300 | GGUCCCUCUGCCUCUGGAG |
| siRNA 250 | 250 | UCCAGAGGCAGAGGGACCC | 2301 | GGGUCCCUCUGCCUCUGGA |
| siRNA 251 | 251 | CCAGAGGCAGAGGGACCCC | 2302 | GGGGUCCCUCUGCCUCUGG |
| siRNA 252 | 252 | CAGAGGCAGAGGGACCCCA | 2303 | UGGGGUCCCUCUGCCUCUG |
| siRNA 253 | 253 | AGAGGCAGAGGGACCCCAA | 2304 | UUGGGGUCCCUCUGCCUCU |
| siRNA 254 | 254 | GAGGCAGAGGGACCCCAAA | 2305 | UUUGGGGUCCCUCUGCCUC |
| siRNA 255 | 255 | AGGCAGAGGGACCCCAAAG | 2306 | CUUUGGGGUCCCUCUGCCU |
| siRNA 256 | 256 | GGCAGAGGGACCCCAAAGU | 2307 | ACUUUGGGGUCCCUCUGCC |
| siRNA 257 | 257 | GCAGAGGGACCCCAAAGUG | 2308 | CACUUUGGGGUCCCUCUGC |
| siRNA 258 | 258 | CAGAGGGACCCCAAAGUGG | 2309 | CCACUUUGGGGUCCCUCUG |
| siRNA 259 | 259 | AGAGGGACCCCAAAGUGGG | 2310 | CCCACUUUGGGGUCCCUCU |
| siRNA 260 | 260 | GAGGGACCCCAAAGUGGGG | 2311 | CCCCACUUUGGGGUCCCUC |
| siRNA 261 | 261 | AGGGACCCCAAAGUGGGGG | 2312 | CCCCCACUUUGGGGUCCCU |
| siRNA 262 | 262 | GGGACCCCAAAGUGGGGGG | 2313 | CCCCCCACUUUGGGGUCCC |
| siRNA 263 | 263 | GGACCCCAAAGUGGGGGGC | 2314 | GCCCCCCACUUUGGGGUCC |
| siRNA 264 | 264 | GACCCCAAAGUGGGGGGCU | 2315 | AGCCCCCCACUUUGGGGUC |
| siRNA 265 | 265 | ACCCCAAAGUGGGGGGCUC | 2316 | GAGCCCCCCACUUUGGGGU |
| siRNA 266 | 266 | CCCCAAAGUGGGGGGCUCC | 2317 | GGAGCCCCCCACUUUGGGG |
| siRNA 267 | 267 | CCCAAAGUGGGGGGCUCCC | 2318 | GGGAGCCCCCCACUUUGGG |
| siRNA 268 | 268 | CCAAAGUGGGGGGCUCCCG | 2319 | CGGGAGCCCCCCACUUUGG |
| siRNA 269 | 269 | CAAAGUGGGGGGCUCCCGC | 2320 | GCGGGAGCCCCCCACUUUG |
| siRNA 270 | 270 | AAAGUGGGGGGCUCCCGCC | 2321 | GGCGGGAGCCCCCCACUUU |
| siRNA 271 | 271 | AAGUGGGGGGCUCCCGCCC | 2322 | GGGCGGGAGCCCCCCACUU |
| siRNA 272 | 272 | AGUGGGGGGCUCCCGCCCC | 2323 | GGGGCGGGAGCCCCCCACU |
| siRNA 273 | 273 | GUGGGGGGCUCCCGCCCCC | 2324 | GGGGGCGGGAGCCCCCCAC |
| siRNA 274 | 274 | UGGGGGGCUCCCGCCCCCG | 2325 | CGGGGGCGGGAGCCCCCCA |
| siRNA 275 | 275 | GGGGGGCUCCCGCCCCCGC | 2326 | GCGGGGGCGGGAGCCCCCC |
| siRNA 276 | 276 | GGGGGCUCCCGCCCCCGCC | 2327 | GGCGGGGGCGGGAGCCCCC |
| siRNA 277 | 277 | GGGGCUCCCGCCCCCGCCC | 2328 | GGGCGGGGGCGGGAGCCCC |
| siRNA 278 | 278 | GGGCUCCCGCCCCCGCCCA | 2329 | UGGGCGGGGGCGGGAGCCC |
| siRNA 279 | 279 | GGCUCCCGCCCCCGCCCAG | 2330 | CUGGGCGGGGGCGGGAGCC |
| siRNA 280 | 280 | GCUCCCGCCCCCGCCCAGG | 2331 | CCUGGGCGGGGGCGGGAGC |
| siRNA 281 | 281 | CUCCCGCCCCCGCCCAGGG | 2332 | CCCUGGGCGGGGGCGGGAG |
| siRNA 282 | 282 | UCCCGCCCCCGCCCAGGGC | 2333 | GCCCUGGGCGGGGGCGGGA |
| siRNA 283 | 283 | CCCGCCCCCGCCCAGGGCA | 2334 | UGCCCUGGGCGGGGGCGGG |
| siRNA 284 | 284 | CCGCCCCCGCCCAGGGCAG | 2335 | CUGCCCUGGGCGGGGGCGG |
| siRNA 285 | 285 | CGCCCCCGCCCAGGGCAGU | 2336 | ACUGCCCUGGGCGGGGGCG |
| siRNA 286 | 286 | GCCCCCGCCCAGGGCAGUU | 2337 | AACUGCCCUGGGCGGGGGC |
| siRNA 287 | 287 | CCCCCGCCCAGGGCAGUUC | 2338 | GAACUGCCCUGGGCGGGGG |
| siRNA 288 | 288 | CCCCGCCCAGGGCAGUUCC | 2339 | GGAACUGCCCUGGGCGGGG |
| siRNA 289 | 289 | CCCGCCCAGGGCAGUUCCC | 2340 | GGGAACUGCCCUGGGCGGG |
| siRNA 290 | 290 | CCGCCCAGGGCAGUUCCCU | 2341 | AGGGAACUGCCCUGGGCGG |
| siRNA 291 | 291 | CGCCCAGGGCAGUUCCCUC | 2342 | GAGGGAACUGCCCUGGGCG |
| siRNA 292 | 292 | GCCCAGGGCAGUUCCCUCG | 2343 | CGAGGGAACUGCCCUGGGC |
| siRNA 293 | 293 | CCCAGGGCAGUUCCCUCGA | 2344 | UCGAGGGAACUGCCCUGGG |
| siRNA 294 | 294 | CCAGGGCAGUUCCCUCGAG | 2345 | CUCGAGGGAACUGCCCUGG |
| siRNA 295 | 295 | CAGGGCAGUUCCCUCGAGC | 2346 | GCUCGAGGGAACUGCCCUG |
| siRNA 296 | 296 | AGGGCAGUUCCCUCGAGCA | 2347 | UGCUCGAGGGAACUGCCCU |
| siRNA 297 | 297 | GGGCAGUUCCCUCGAGCAG | 2348 | CUGCUCGAGGGAACUGCCC |
| siRNA 298 | 298 | GGCAGUUCCCUCGAGCAGU | 2349 | ACUGCUCGAGGGAACUGCC |
| siRNA 299 | 299 | GCAGUUCCCUCGAGCAGUA | 2350 | UACUGCUCGAGGGAACUGC |
| siRNA 300 | 300 | CAGUUCCCUCGAGCAGUAG | 2351 | CUACUGCUCGAGGGAACUG |
| siRNA 301 | 301 | AGUUCCCUCGAGCAGUAGC | 2352 | GCUACUGCUCGAGGGAACU |
| siRNA 302 | 302 | GUUCCCUCGAGCAGUAGCC | 2353 | GGCUACUGCUCGAGGGAAC |
| siRNA 303 | 303 | UUCCCUCGAGCAGUAGCCC | 2354 | GGGCUACUGCUCGAGGGAA |
| siRNA 304 | 304 | UCCCUCGAGCAGUAGCCCC | 2355 | GGGGCUACUGCUCGAGGGA |
| siRNA 305 | 305 | CCCUCGAGCAGUAGCCCCC | 2356 | GGGGGCUACUGCUCGAGGG |
| siRNA 306 | 306 | CCUCGAGCAGUAGCCCCCA | 2357 | UGGGGGCUACUGCUCGAGG |
| siRNA 307 | 307 | CUCGAGCAGUAGCCCCCAG | 2358 | CUGGGGGCUACUGCUCGAG |
| siRNA 308 | 308 | UCGAGCAGUAGCCCCCAGG | 2359 | CCUGGGGGCUACUGCUCGA |
| siRNA 309 | 309 | CGAGCAGUAGCCCCCAGGC | 2360 | GCCUGGGGGCUACUGCUCG |
| siRNA 310 | 310 | GAGCAGUAGCCCCCAGGCC | 2361 | GGCCUGGGGGCUACUGCUC |
| siRNA 311 | 311 | AGCAGUAGCCCCCAGGCCC | 2362 | GGGCCUGGGGGCUACUGCU |
| siRNA 312 | 312 | GCAGUAGCCCCCAGGCCCA | 2363 | UGGGCCUGGGGGCUACUGC |
| siRNA 313 | 313 | CAGUAGCCCCCAGGCCCAA | 2364 | UUGGGCCUGGGGGCUACUG |
| siRNA 314 | 314 | AGUAGCCCCCAGGCCCAAG | 2365 | CUUGGGCCUGGGGGCUACU |
| siRNA 315 | 315 | GUAGCCCCCAGGCCCAAGC | 2366 | GCUUGGGCCUGGGGGCUAC |
| siRNA 316 | 316 | UAGCCCCCAGGCCCAAGCA | 2367 | UGCUUGGGCCUGGGGGCUA |
| siRNA 317 | 317 | AGCCCCCAGGCCCAAGCAC | 2368 | GUGCUUGGGCCUGGGGGCU |
| siRNA 318 | 318 | GCCCCCAGGCCCAAGCACU | 2369 | AGUGCUUGGGCCUGGGGGC |
| siRNA 319 | 319 | CCCCCAGGCCCAAGCACUC | 2370 | GAGUGCUUGGGCCUGGGGG |
| siRNA 320 | 320 | CCCCAGGCCCAAGCACUCA | 2371 | UGAGUGCUUGGGCCUGGGG |
| siRNA 321 | 321 | CCCAGGCCCAAGCACUCAC | 2372 | GUGAGUGCUUGGGCCUGGG |
| siRNA 322 | 322 | CCAGGCCCAAGCACUCACC | 2373 | GGUGAGUGCUUGGGCCUGG |
| siRNA 323 | 323 | CAGGCCCAAGCACUCACCG | 2374 | CGGUGAGUGCUUGGGCCUG |
| siRNA 324 | 324 | AGGCCCAAGCACUCACCGA | 2375 | UCGGUGAGUGCUUGGGCCU |
| siRNA 325 | 325 | GGCCCAAGCACUCACCGAG | 2376 | CUCGGUGAGUGCUUGGGCC |
| siRNA 326 | 326 | GCCCAAGCACUCACCGAGG | 2377 | CCUCGGUGAGUGCUUGGGC |
| siRNA 327 | 327 | CCCAAGCACUCACCGAGGA | 2378 | UCCUCGGUGAGUGCUUGGG |
| siRNA 328 | 328 | CCAAGCACUCACCGAGGAC | 2379 | GUCCUCGGUGAGUGCUUGG |
| siRNA 329 | 329 | CAAGCACUCACCGAGGACG | 2380 | CGUCCUCGGUGAGUGCUUG |
| siRNA 330 | 330 | AAGCACUCACCGAGGACGG | 2381 | CCGUCCUCGGUGAGUGCUU |
| siRNA 331 | 331 | AGCACUCACCGAGGACGGG | 2382 | CCCGUCCUCGGUGAGUGCU |
| siRNA 332 | 332 | GCACUCACCGAGGACGGGA | 2383 | UCCCGUCCUCGGUGAGUGC |
| siRNA 333 | 333 | CACUCACCGAGGACGGGAG | 2384 | CUCCCGUCCUCGGUGAGUG |
| siRNA 334 | 334 | ACUCACCGAGGACGGGAGG | 2385 | CCUCCCGUCCUCGGUGAGU |
| siRNA 335 | 335 | CUCACCGAGGACGGGAGGC | 2386 | GCCUCCCGUCCUCGGUGAG |
| siRNA 336 | 336 | UCACCGAGGACGGGAGGCC | 2387 | GGCCUCCCGUCCUCGGUGA |
| siRNA 337 | 337 | CACCGAGGACGGGAGGCCC | 2388 | GGGCCUCCCGUCCUCGGUG |
| siRNA 338 | 338 | ACCGAGGACGGGAGGCCCU | 2389 | AGGGCCUCCCGUCCUCGGU |
| siRNA 339 | 339 | CCGAGGACGGGAGGCCCUG | 2390 | CAGGGCCUCCCGUCCUCGG |
| siRNA 340 | 340 | CGAGGACGGGAGGCCCUGC | 2391 | GCAGGGCCUCCCGUCCUCG |
| siRNA 341 | 341 | GAGGACGGGAGGCCCUGCA | 2392 | UGCAGGGCCUCCCGUCCUC |
| siRNA 342 | 342 | AGGACGGGAGGCCCUGCAG | 2393 | CUGCAGGGCCUCCCGUCCU |
| siRNA 343 | 343 | GGACGGGAGGCCCUGCAGG | 2394 | CCUGCAGGGCCUCCCGUCC |
| siRNA 344 | 344 | GACGGGAGGCCCUGCAGGU | 2395 | ACCUGCAGGGCCUCCCGUC |
| siRNA 345 | 345 | ACGGGAGGCCCUGCAGGUU | 2396 | AACCUGCAGGGCCUCCCGU |
| siRNA 346 | 346 | CGGGAGGCCCUGCAGGUUC | 2397 | GAACCUGCAGGGCCUCCCG |
| siRNA 347 | 347 | GGGAGGCCCUGCAGGUUCC | 2398 | GGAACCUGCAGGGCCUCCC |
| siRNA 348 | 348 | GGAGGCCCUGCAGGUUCCC | 2399 | GGGAACCUGCAGGGCCUCC |
| siRNA 349 | 349 | GAGGCCCUGCAGGUUCCCC | 2400 | GGGGAACCUGCAGGGCCUC |
| siRNA 350 | 350 | AGGCCCUGCAGGUUCCCCU | 2401 | AGGGGAACCUGCAGGGCCU |
| siRNA 351 | 351 | GGCCCUGCAGGUUCCCCUU | 2402 | AAGGGGAACCUGCAGGGCC |
| siRNA 352 | 352 | GCCCUGCAGGUUCCCCUUC | 2403 | GAAGGGGAACCUGCAGGGC |
| siRNA 353 | 353 | CCCUGCAGGUUCCCCUUCC | 2404 | GGAAGGGGAACCUGCAGGG |
| siRNA 354 | 354 | CCUGCAGGUUCCCCUUCCG | 2405 | CGGAAGGGGAACCUGCAGG |
| siRNA 355 | 355 | CUGCAGGUUCCCCUUCCGC | 2406 | GCGGAAGGGGAACCUGCAG |
| siRNA 356 | 356 | UGCAGGUUCCCCUUCCGCU | 2407 | AGCGGAAGGGGAACCUGCA |
| siRNA 357 | 357 | GCAGGUUCCCCUUCCGCUA | 2408 | UAGCGGAAGGGGAACCUGC |
| siRNA 358 | 358 | CAGGUUCCCCUUCCGCUAC | 2409 | GUAGCGGAAGGGGAACCUG |
| siRNA 359 | 359 | AGGUUCCCCUUCCGCUACG | 2410 | CGUAGCGGAAGGGGAACCU |
| siRNA 360 | 360 | GGUUCCCCUUCCGCUACGG | 2411 | CCGUAGCGGAAGGGGAACC |
| siRNA 361 | 361 | GUUCCCCUUCCGCUACGGG | 2412 | CCCGUAGCGGAAGGGGAAC |
| siRNA 362 | 362 | UUCCCCUUCCGCUACGGGG | 2413 | CCCCGUAGCGGAAGGGGAA |
| siRNA 363 | 363 | UCCCCUUCCGCUACGGGGG | 2414 | CCCCCGUAGCGGAAGGGGA |
| siRNA 364 | 364 | CCCCUUCCGCUACGGGGGC | 2415 | GCCCCCGUAGCGGAAGGGG |
| siRNA 365 | 365 | CCCUUCCGCUACGGGGGCC | 2416 | GGCCCCCGUAGCGGAAGGG |
| siRNA 366 | 366 | CCUUCCGCUACGGGGGCCG | 2417 | CGGCCCCCGUAGCGGAAGG |
| siRNA 367 | 367 | CUUCCGCUACGGGGGCCGC | 2418 | GCGGCCCCCGUAGCGGAAG |
| siRNA 368 | 368 | UUCCGCUACGGGGGCCGCA | 2419 | UGCGGCCCCCGUAGCGGAA |
| siRNA 369 | 369 | UCCGCUACGGGGGCCGCAU | 2420 | AUGCGGCCCCCGUAGCGGA |
| siRNA 370 | 370 | CCGCUACGGGGGCCGCAUG | 2421 | CAUGCGGCCCCCGUAGCGG |
| siRNA 371 | 371 | CGCUACGGGGGCCGCAUGC | 2422 | GCAUGCGGCCCCCGUAGCG |
| siRNA 372 | 372 | GCUACGGGGGCCGCAUGCU | 2423 | AGCAUGCGGCCCCCGUAGC |
| siRNA 373 | 373 | CUACGGGGGCCGCAUGCUG | 2424 | CAGCAUGCGGCCCCCGUAG |
| siRNA 374 | 374 | UACGGGGGCCGCAUGCUGC | 2425 | GCAGCAUGCGGCCCCCGUA |
| siRNA 375 | 375 | ACGGGGGCCGCAUGCUGCA | 2426 | UGCAGCAUGCGGCCCCCGU |
| siRNA 376 | 376 | CGGGGGCCGCAUGCUGCAU | 2427 | AUGCAGCAUGCGGCCCCCG |
| siRNA 377 | 377 | GGGGGCCGCAUGCUGCAUG | 2428 | CAUGCAGCAUGCGGCCCCC |
| siRNA 378 | 378 | GGGGCCGCAUGCUGCAUGC | 2429 | GCAUGCAGCAUGCGGCCCC |
| siRNA 379 | 379 | GGGCCGCAUGCUGCAUGCC | 2430 | GGCAUGCAGCAUGCGGCCC |
| siRNA 380 | 380 | GGCCGCAUGCUGCAUGCCU | 2431 | AGGCAUGCAGCAUGCGGCC |
| siRNA 381 | 381 | GCCGCAUGCUGCAUGCCUG | 2432 | CAGGCAUGCAGCAUGCGGC |
| siRNA 382 | 382 | CCGCAUGCUGCAUGCCUGC | 2433 | GCAGGCAUGCAGCAUGCGG |
| siRNA 383 | 383 | CGCAUGCUGCAUGCCUGCA | 2434 | UGCAGGCAUGCAGCAUGCG |
| siRNA 384 | 384 | GCAUGCUGCAUGCCUGCAC | 2435 | GUGCAGGCAUGCAGCAUGC |
| siRNA 385 | 385 | CAUGCUGCAUGCCUGCACU | 2436 | AGUGCAGGCAUGCAGCAUG |
| siRNA 386 | 386 | AUGCUGCAUGCCUGCACUU | 2437 | AAGUGCAGGCAUGCAGCAU |
| siRNA 387 | 387 | UGCUGCAUGCCUGCACUUC | 2438 | GAAGUGCAGGCAUGCAGCA |
| siRNA 388 | 388 | GCUGCAUGCCUGCACUUCG | 2439 | CGAAGUGCAGGCAUGCAGC |
| siRNA 389 | 389 | CUGCAUGCCUGCACUUCGG | 2440 | CCGAAGUGCAGGCAUGCAG |
| siRNA 390 | 390 | UGCAUGCCUGCACUUCGGA | 2441 | UCCGAAGUGCAGGCAUGCA |
| siRNA 391 | 391 | GCAUGCCUGCACUUCGGAG | 2442 | CUCCGAAGUGCAGGCAUGC |
| siRNA 392 | 392 | CAUGCCUGCACUUCGGAGG | 2443 | CCUCCGAAGUGCAGGCAUG |
| siRNA 393 | 393 | AUGCCUGCACUUCGGAGGG | 2444 | CCCUCCGAAGUGCAGGCAU |
| siRNA 394 | 394 | UGCCUGCACUUCGGAGGGC | 2445 | GCCCUCCGAAGUGCAGGCA |
| siRNA 395 | 395 | GCCUGCACUUCGGAGGGCA | 2446 | UGCCCUCCGAAGUGCAGGC |
| siRNA 396 | 396 | CCUGCACUUCGGAGGGCAG | 2447 | CUGCCCUCCGAAGUGCAGG |
| siRNA 397 | 397 | CUGCACUUCGGAGGGCAGU | 2448 | ACUGCCCUCCGAAGUGCAG |
| siRNA 398 | 398 | UGCACUUCGGAGGGCAGUG | 2449 | CACUGCCCUCCGAAGUGCA |
| siRNA 399 | 399 | GCACUUCGGAGGGCAGUGC | 2450 | GCACUGCCCUCCGAAGUGC |
| siRNA 400 | 400 | CACUUCGGAGGGCAGUGCA | 2451 | UGCACUGCCCUCCGAAGUG |
| siRNA 401 | 401 | ACUUCGGAGGGCAGUGCAC | 2452 | GUGCACUGCCCUCCGAAGU |
| siRNA 402 | 402 | CUUCGGAGGGCAGUGCACA | 2453 | UGUGCACUGCCCUCCGAAG |
| siRNA 403 | 403 | UUCGGAGGGCAGUGCACAC | 2454 | GUGUGCACUGCCCUCCGAA |
| siRNA 404 | 404 | UCGGAGGGCAGUGCACACA | 2455 | UGUGUGCACUGCCCUCCGA |
| siRNA 405 | 405 | CGGAGGGCAGUGCACACAG | 2456 | CUGUGUGCACUGCCCUCCG |
| siRNA 406 | 406 | GGAGGGCAGUGCACACAGG | 2457 | CCUGUGUGCACUGCCCUCC |
| siRNA 407 | 407 | GAGGGCAGUGCACACAGGA | 2458 | UCCUGUGUGCACUGCCCUC |
| siRNA 408 | 408 | AGGGCAGUGCACACAGGAA | 2459 | UUCCUGUGUGCACUGCCCU |
| siRNA 409 | 409 | GGGCAGUGCACACAGGAAG | 2460 | CUUCCUGUGUGCACUGCCC |
| siRNA 410 | 410 | GGCAGUGCACACAGGAAGU | 2461 | ACUUCCUGUGUGCACUGCC |
| siRNA 411 | 411 | GCAGUGCACACAGGAAGUG | 2462 | CACUUCCUGUGUGCACUGC |
| siRNA 412 | 412 | CAGUGCACACAGGAAGUGG | 2463 | CCACUUCCUGUGUGCACUG |
| siRNA 413 | 413 | AGUGCACACAGGAAGUGGU | 2464 | ACCACUUCCUGUGUGCACU |
| siRNA 414 | 414 | GUGCACACAGGAAGUGGUG | 2465 | CACCACUUCCUGUGUGCAC |
| siRNA 415 | 415 | UGCACACAGGAAGUGGUGU | 2466 | ACACCACUUCCUGUGUGCA |
| siRNA 416 | 416 | GCACACAGGAAGUGGUGUG | 2467 | CACACCACUUCCUGUGUGC |
| siRNA 417 | 417 | CACACAGGAAGUGGUGUGC | 2468 | GCACACCACUUCCUGUGUG |
| siRNA 418 | 418 | ACACAGGAAGUGGUGUGCC | 2469 | GGCACACCACUUCCUGUGU |
| siRNA 419 | 419 | CACAGGAAGUGGUGUGCCA | 2470 | UGGCACACCACUUCCUGUG |
| siRNA 420 | 420 | ACAGGAAGUGGUGUGCCAC | 2471 | GUGGCACACCACUUCCUGU |
| siRNA 421 | 421 | CAGGAAGUGGUGUGCCACA | 2472 | UGUGGCACACCACUUCCUG |
| siRNA 422 | 422 | AGGAAGUGGUGUGCCACAA | 2473 | UUGUGGCACACCACUUCCU |
| siRNA 423 | 423 | GGAAGUGGUGUGCCACAAC | 2474 | GUUGUGGCACACCACUUCC |
| siRNA 424 | 424 | GAAGUGGUGUGCCACAACU | 2475 | AGUUGUGGCACACCACUUC |
| siRNA 425 | 425 | AAGUGGUGUGCCACAACUC | 2476 | GAGUUGUGGCACACCACUU |
| siRNA 426 | 426 | AGUGGUGUGCCACAACUCA | 2477 | UGAGUUGUGGCACACCACU |
| siRNA 427 | 427 | GUGGUGUGCCACAACUCAC | 2478 | GUGAGUUGUGGCACACCAC |
| siRNA 428 | 428 | UGGUGUGCCACAACUCACA | 2479 | UGUGAGUUGUGGCACACCA |
| siRNA 429 | 429 | GGUGUGCCACAACUCACAA | 2480 | UUGUGAGUUGUGGCACACC |
| siRNA 430 | 430 | GUGUGCCACAACUCACAAC | 2481 | GUUGUGAGUUGUGGCACAC |
| siRNA 431 | 431 | UGUGCCACAACUCACAACU | 2482 | AGUUGUGAGUUGUGGCACA |
| siRNA 432 | 432 | GUGCCACAACUCACAACUA | 2483 | UAGUUGUGAGUUGUGGCAC |
| siRNA 433 | 433 | UGCCACAACUCACAACUAC | 2484 | GUAGUUGUGAGUUGUGGCA |
| siRNA 434 | 434 | GCCACAACUCACAACUACG | 2485 | CGUAGUUGUGAGUUGUGGC |
| siRNA 435 | 435 | CCACAACUCACAACUACGA | 2486 | UCGUAGUUGUGAGUUGUGG |
| siRNA 436 | 436 | CACAACUCACAACUACGAC | 2487 | GUCGUAGUUGUGAGUUGUG |
| siRNA 437 | 437 | ACAACUCACAACUACGACC | 2488 | GGUCGUAGUUGUGAGUUGU |
| siRNA 438 | 438 | CAACUCACAACUACGACCG | 2489 | CGGUCGUAGUUGUGAGUUG |
| siRNA 439 | 439 | AACUCACAACUACGACCGG | 2490 | CCGGUCGUAGUUGUGAGUU |
| siRNA 440 | 440 | ACUCACAACUACGACCGGG | 2491 | CCCGGUCGUAGUUGUGAGU |
| siRNA 441 | 441 | CUCACAACUACGACCGGGA | 2492 | UCCCGGUCGUAGUUGUGAG |
| siRNA 442 | 442 | UCACAACUACGACCGGGAC | 2493 | GUCCCGGUCGUAGUUGUGA |
| siRNA 443 | 443 | CACAACUACGACCGGGACA | 2494 | UGUCCCGGUCGUAGUUGUG |
| siRNA 444 | 444 | ACAACUACGACCGGGACAG | 2495 | CUGUCCCGGUCGUAGUUGU |
| siRNA 445 | 445 | CAACUACGACCGGGACAGG | 2496 | CCUGUCCCGGUCGUAGUUG |
| siRNA 446 | 446 | AACUACGACCGGGACAGGG | 2497 | CCCUGUCCCGGUCGUAGUU |
| siRNA 447 | 447 | ACUACGACCGGGACAGGGC | 2498 | GCCCUGUCCCGGUCGUAGU |
| siRNA 448 | 448 | CUACGACCGGGACAGGGCC | 2499 | GGCCCUGUCCCGGUCGUAG |
| siRNA 449 | 449 | UACGACCGGGACAGGGCCU | 2500 | AGGCCCUGUCCCGGUCGUA |
| siRNA 450 | 450 | ACGACCGGGACAGGGCCUG | 2501 | CAGGCCCUGUCCCGGUCGU |
| siRNA 451 | 451 | CGACCGGGACAGGGCCUGG | 2502 | CCAGGCCCUGUCCCGGUCG |
| siRNA 452 | 452 | GACCGGGACAGGGCCUGGG | 2503 | CCCAGGCCCUGUCCCGGUC |
| siRNA 453 | 453 | ACCGGGACAGGGCCUGGGG | 2504 | CCCCAGGCCCUGUCCCGGU |
| siRNA 454 | 454 | CCGGGACAGGGCCUGGGGC | 2505 | GCCCCAGGCCCUGUCCCGG |
| siRNA 455 | 455 | CGGGACAGGGCCUGGGGCU | 2506 | AGCCCCAGGCCCUGUCCCG |
| siRNA 456 | 456 | GGGACAGGGCCUGGGGCUA | 2507 | UAGCCCCAGGCCCUGUCCC |
| siRNA 457 | 457 | GGACAGGGCCUGGGGCUAC | 2508 | GUAGCCCCAGGCCCUGUCC |
| siRNA 458 | 458 | GACAGGGCCUGGGGCUACU | 2509 | AGUAGCCCCAGGCCCUGUC |
| siRNA 459 | 459 | ACAGGGCCUGGGGCUACUG | 2510 | CAGUAGCCCCAGGCCCUGU |
| siRNA 460 | 460 | CAGGGCCUGGGGCUACUGU | 2511 | ACAGUAGCCCCAGGCCCUG |
| siRNA 461 | 461 | AGGGCCUGGGGCUACUGUG | 2512 | CACAGUAGCCCCAGGCCCU |
| siRNA 462 | 462 | GGGCCUGGGGCUACUGUGU | 2513 | ACACAGUAGCCCCAGGCCC |
| siRNA 463 | 463 | GGCCUGGGGCUACUGUGUG | 2514 | CACACAGUAGCCCCAGGCC |
| siRNA 464 | 464 | GCCUGGGGCUACUGUGUGG | 2515 | CCACACAGUAGCCCCAGGC |
| siRNA 465 | 465 | CCUGGGGCUACUGUGUGGA | 2516 | UCCACACAGUAGCCCCAGG |
| siRNA 466 | 466 | CUGGGGCUACUGUGUGGAG | 2517 | CUCCACACAGUAGCCCCAG |
| siRNA 467 | 467 | UGGGGCUACUGUGUGGAGG | 2518 | CCUCCACACAGUAGCCCCA |
| siRNA 468 | 468 | GGGGCUACUGUGUGGAGGC | 2519 | GCCUCCACACAGUAGCCCC |
| siRNA 469 | 469 | GGGCUACUGUGUGGAGGCC | 2520 | GGCCUCCACACAGUAGCCC |
| siRNA 470 | 470 | GGCUACUGUGUGGAGGCCA | 2521 | UGGCCUCCACACAGUAGCC |
| siRNA 471 | 471 | GCUACUGUGUGGAGGCCAC | 2522 | GUGGCCUCCACACAGUAGC |
| siRNA 472 | 472 | CUACUGUGUGGAGGCCACC | 2523 | GGUGGCCUCCACACAGUAG |
| siRNA 473 | 473 | UACUGUGUGGAGGCCACCC | 2524 | GGGUGGCCUCCACACAGUA |
| siRNA 474 | 474 | ACUGUGUGGAGGCCACCCC | 2525 | GGGGUGGCCUCCACACAGU |
| siRNA 475 | 475 | CUGUGUGGAGGCCACCCCG | 2526 | CGGGGUGGCCUCCACACAG |
| siRNA 476 | 476 | UGUGUGGAGGCCACCCCGC | 2527 | GCGGGGUGGCCUCCACACA |
| siRNA 477 | 477 | GUGUGGAGGCCACCCCGCC | 2528 | GGCGGGGUGGCCUCCACAC |
| siRNA 478 | 478 | UGUGGAGGCCACCCCGCCU | 2529 | AGGCGGGGUGGCCUCCACA |
| siRNA 479 | 479 | GUGGAGGCCACCCCGCCUC | 2530 | GAGGCGGGGUGGCCUCCAC |
| siRNA 480 | 480 | UGGAGGCCACCCCGCCUCC | 2531 | GGAGGCGGGGUGGCCUCCA |
| siRNA 481 | 481 | GGAGGCCACCCCGCCUCCA | 2532 | UGGAGGCGGGGUGGCCUCC |
| siRNA 482 | 482 | GAGGCCACCCCGCCUCCAG | 2533 | CUGGAGGCGGGGUGGCCUC |
| siRNA 483 | 483 | AGGCCACCCCGCCUCCAGG | 2534 | CCUGGAGGCGGGGUGGCCU |
| siRNA 484 | 484 | GGCCACCCCGCCUCCAGGG | 2535 | CCCUGGAGGCGGGGUGGCC |
| siRNA 485 | 485 | GCCACCCCGCCUCCAGGGG | 2536 | CCCCUGGAGGCGGGGUGGC |
| siRNA 486 | 486 | CCACCCCGCCUCCAGGGGG | 2537 | CCCCCUGGAGGGCGGGUGG |
| siRNA 487 | 487 | CACCCCGCCUCCAGGGGGC | 2538 | GCCCCCUGGAGGGCGGGUG |
| siRNA 488 | 488 | ACCCCGCCUCCAGGGGGCC | 2539 | GGCCCCCUGGAGGCGGGGU |
| siRNA 489 | 489 | CCCCGCCUCCAGGGGGCCC | 2540 | GGGCCCCCUGGAGGCGGGG |
| siRNA 490 | 490 | CCCGCCUCCAGGGGGCCCA | 2541 | UGGGCCCCCUGGAGGCGGG |
| siRNA 491 | 491 | CCGCCUCCAGGGGGCCCAG | 2542 | CUGGGCCCCCUGGAGGCGG |
| siRNA 492 | 492 | CGCCUCCAGGGGGCCCAGC | 2543 | GCUGGGCCCCCUGGAGGCG |
| siRNA 493 | 493 | GCCUCCAGGGGGCCCAGCU | 2544 | AGCUGGGCCCCCUGGAGGC |
| siRNA 494 | 494 | CCUCCAGGGGGCCCAGCUG | 2545 | CAGCUGGGCCCCCUGGAGG |
| siRNA 495 | 495 | CUCCAGGGGGCCCAGCUGC | 2546 | GCAGCUGGGCCCCCUGGAG |
| siRNA 496 | 496 | UCCAGGGGGCCCAGCUGCC | 2547 | GGCAGCUGGGCCCCCUGGA |
| siRNA 497 | 497 | CCAGGGGGCCCAGCUGCCC | 2548 | GGGCAGCUGGGCCCCCUGG |
| siRNA 498 | 498 | CAGGGGGCCCAGCUGCCCU | 2549 | AGGGCAGCUGGGCCCCCUG |
| siRNA 499 | 499 | AGGGGGCCCAGCUGCCCUG | 2550 | CAGGGCAGCUGGGCCCCCU |
| siRNA 500 | 500 | GGGGGCCCAGCUGCCCUGG | 2551 | CCAGGGCAGCUGGGCCCCC |
| siRNA 501 | 501 | GGGGCCCAGCUGCCCUGGA | 2552 | UCCAGGGCAGCUGGGCCCC |
| siRNA 502 | 502 | GGGCCCAGCUGCCCUGGAU | 2553 | AUCCAGGGCAGCUGGGCCC |
| siRNA 503 | 503 | GGCCCAGCUGCCCUGGAUC | 2554 | GAUCCAGGGCAGCUGGGCC |
| siRNA 504 | 504 | GCCCAGCUGCCCUGGAUCC | 2555 | GGAUCCAGGGCAGCUGGGC |
| siRNA 505 | 505 | CCCAGCUGCCCUGGAUCCC | 2556 | GGGAUCCAGGGCAGCUGGG |
| siRNA 506 | 506 | CCAGCUGCCCUGGAUCCCU | 2557 | AGGGAUCCAGGGCAGCUGG |
| siRNA 507 | 507 | CAGCUGCCCUGGAUCCCUG | 2558 | CAGGGAUCCAGGGCAGCUG |
| siRNA 508 | 508 | AGCUGCCCUGGAUCCCUGU | 2559 | ACAGGGAUCCAGGGCAGCU |
| siRNA 509 | 509 | GCUGCCCUGGAUCCCUGUG | 2560 | CACAGGGAUCCAGGGCAGC |
| siRNA 510 | 510 | CUGCCCUGGAUCCCUGUGC | 2561 | GCACAGGGAUCCAGGGCAG |
| siRNA 511 | 511 | UGCCCUGGAUCCCUGUGCC | 2562 | GGCACAGGGAUCCAGGGCA |
| siRNA 512 | 512 | GCCCUGGAUCCCUGUGCCU | 2563 | AGGCACAGGGAUCCAGGGC |
| siRNA 513 | 513 | CCCUGGAUCCCUGUGCCUC | 2564 | GAGGCACAGGGAUCCAGGG |
| siRNA 514 | 514 | CCUGGAUCCCUGUGCCUCC | 2565 | GGAGGCACAGGGAUCCAGG |
| siRNA 515 | 515 | CUGGAUCCCUGUGCCUCCG | 2566 | CGGAGGCACAGGGAUCCAG |
| siRNA 516 | 516 | UGGAUCCCUGUGCCUCCGG | 2567 | CCGGAGGCACAGGGAUCCA |
| siRNA 517 | 517 | GGAUCCCUGUGCCUCCGGC | 2568 | GCCGGAGGCACAGGGAUCC |
| siRNA 518 | 518 | GAUCCCUGUGCCUCCGGCC | 2569 | GGCCGGAGGCACAGGGAUC |
| siRNA 519 | 519 | AUCCCUGUGCCUCCGGCCC | 2570 | GGGCCGGAGGCACAGGGAU |
| siRNA 520 | 520 | UCCCUGUGCCUCCGGCCCC | 2571 | GGGGCCGGAGGCACAGGGA |
| siRNA 521 | 521 | CCCUGUGCCUCCGGCCCCU | 2572 | AGGGGCCGGAGGCACAGGG |
| siRNA 522 | 522 | CCUGUGCCUCCGGCCCCUG | 2573 | CAGGGGCCGGAGGCACAGG |
| siRNA 523 | 523 | CUGUGCCUCCGGCCCCUGC | 2574 | GCAGGGGCCGGAGGCACAG |
| siRNA 524 | 524 | UGUGCCUCCGGCCCCUGCC | 2575 | GGCAGGGGCCGGAGGCACA |
| siRNA 525 | 525 | GUGCCUCCGGCCCCUGCCU | 2576 | AGGCAGGGGCCGGAGGCAC |
| siRNA 526 | 526 | UGCCUCCGGCCCCUGCCUC | 2577 | GAGGCAGGGGCCGGAGGCA |
| siRNA 527 | 527 | GCCUCCGGCCCCUGCCUCA | 2578 | UGAGGCAGGGGCCGGAGGC |
| siRNA 528 | 528 | CCUCCGGCCCCUGCCUCAA | 2579 | UUGAGGCAGGGGCCGGAGG |
| siRNA 529 | 529 | CUCCGGCCCCUGCCUCAAU | 2580 | AUUGAGGCAGGGGCCGGAG |
| siRNA 530 | 530 | UCCGGCCCCUGCCUCAAUG | 2581 | CAUUGAGGCAGGGGCCGGA |
| siRNA 531 | 531 | CCGGCCCCUGCCUCAAUGG | 2582 | CCAUUGAGGCAGGGGCCGG |
| siRNA 532 | 532 | CGGCCCCUGCCUCAAUGGA | 2583 | UCCAUUGAGGCAGGGGCCG |
| siRNA 533 | 533 | GGCCCCUGCCUCAAUGGAG | 2584 | CUCCAUUGAGGCAGGGGCC |
| siRNA 534 | 534 | GCCCCUGCCUCAAUGGAGG | 2585 | CCUCCAUUGAGGCAGGGGC |
| siRNA 535 | 535 | CCCCUGCCUCAAUGGAGGC | 2586 | GCCUCCAUUGAGGCAGGGG |
| siRNA 536 | 536 | CCCUGCCUCAAUGGAGGCU | 2587 | AGCCUCCAUUGAGGCAGGG |
| siRNA 537 | 537 | CCUGCCUCAAUGGAGGCUC | 2588 | GAGCCUCCAUUGAGGCAGG |
| siRNA 538 | 538 | CUGCCUCAAUGGAGGCUCC | 2589 | GGAGCCUCCAUUGAGGCAG |
| siRNA 539 | 539 | UGCCUCAAUGGAGGCUCCU | 2590 | AGGAGCCUCCAUUGAGGCA |
| siRNA 540 | 540 | GCCUCAAUGGAGGCUCCUG | 2591 | CAGGAGCCUCCAUUGAGGC |
| siRNA 541 | 541 | CCUCAAUGGAGGCUCCUGC | 2592 | GCAGGAGCCUCCAUUGAGG |
| siRNA 542 | 542 | CUCAAUGGAGGCUCCUGCU | 2593 | AGCAGGAGCCUCCAUUGAG |
| siRNA 543 | 543 | UCAAUGGAGGCUCCUGCUC | 2594 | GAGCAGGAGCCUCCAUUGA |
| siRNA 544 | 544 | CAAUGGAGGCUCCUGCUCC | 2595 | GGAGCAGGAGCCUCCAUUG |
| siRNA 545 | 545 | AAUGGAGGCUCCUGCUCCA | 2596 | UGGAGCAGGAGCCUCCAUU |
| siRNA 546 | 546 | AUGGAGGCUCCUGCUCCAA | 2597 | UUGGAGCAGGAGCCUCCAU |
| siRNA 547 | 547 | UGGAGGCUCCUGCUCCAAU | 2598 | AUUGGAGCAGGAGCCUCCA |
| siRNA 548 | 548 | GGAGGCUCCUGCUCCAAUA | 2599 | UAUUGGAGCAGGAGCCUCC |
| siRNA 549 | 549 | GAGGCUCCUGCUCCAAUAC | 2600 | GUAUUGGAGCAGGAGCCUC |
| siRNA 550 | 550 | AGGCUCCUGCUCCAAUACC | 2601 | GGUAUUGGAGCAGGAGCCU |
| siRNA 551 | 551 | GGCUCCUGCUCCAAUACCC | 2602 | GGGUAUUGGAGCAGGAGCC |
| siRNA 552 | 552 | GCUCCUGCUCCAAUACCCA | 2603 | UGGGUAUUGGAGCAGGAGC |
| siRNA 553 | 553 | CUCCUGCUCCAAUACCCAG | 2604 | CUGGGUAUUGGAGCAGGAG |
| siRNA 554 | 554 | UCCUGCUCCAAUACCCAGG | 2605 | CCUGGGUAUUGGAGCAGGA |
| siRNA 555 | 555 | CCUGCUCCAAUACCCAGGA | 2606 | UCCUGGGUAUUGGAGCAGG |
| siRNA 556 | 556 | CUGCUCCAAUACCCAGGAC | 2607 | GUCCUGGGUAUUGGAGCAG |
| siRNA 557 | 557 | UGCUCCAAUACCCAGGACC | 2608 | GGUCCUGGGUAUUGGAGCA |
| siRNA 558 | 558 | GCUCCAAUACCCAGGACCC | 2609 | GGGUCCUGGGUAUUGGAGC |
| siRNA 559 | 559 | CUCCAAUACCCAGGACCCC | 2610 | GGGGUCCUGGGUAUUGGAG |
| siRNA 560 | 560 | UCCAAUACCCAGGACCCCC | 2611 | GGGGGUCCUGGGUAUUGGA |
| siRNA 561 | 561 | CCAAUACCCAGGACCCCCA | 2612 | UGGGGGUCCUGGGUAUUGG |
| siRNA 562 | 562 | CAAUACCCAGGACCCCCAG | 2613 | CUGGGGGUCCUGGGUAUUG |
| siRNA 563 | 563 | AAUACCCAGGACCCCCAGU | 2614 | ACUGGGGGUCCUGGGUAUU |
| siRNA 564 | 564 | AUACCCAGGACCCCCAGUC | 2615 | GACUGGGGGUCCUGGGUAU |
| siRNA 565 | 565 | UACCCAGGACCCCCAGUCC | 2616 | GGACUGGGGGUCCUGGGUA |
| siRNA 566 | 566 | ACCCAGGACCCCCAGUCCU | 2617 | AGGACUGGGGGUCCUGGGU |
| siRNA 567 | 567 | CCCAGGACCCCCAGUCCUA | 2618 | UAGGACUGGGGGUCCUGGG |
| siRNA 568 | 568 | CCAGGACCCCCAGUCCUAU | 2619 | AUAGGACUGGGGGUCCUGG |
| siRNA 569 | 569 | CAGGACCCCCAGUCCUAUC | 2620 | GAUAGGACUGGGGGUCCUG |
| siRNA 570 | 570 | AGGACCCCCAGUCCUAUCA | 2621 | UGAUAGGACUGGGGGUCCU |
| siRNA 571 | 571 | GGACCCCCAGUCCUAUCAC | 2622 | GUGAUAGGACUGGGGGUCC |
| siRNA 572 | 572 | GACCCCCAGUCCUAUCACU | 2623 | AGUGAUAGGACUGGGGGUC |
| siRNA 573 | 573 | ACCCCCAGUCCUAUCACUG | 2624 | CAGUGAUAGGACUGGGGGU |
| siRNA 574 | 574 | CCCCCAGUCCUAUCACUGC | 2625 | GCAGUGAUAGGACUGGGGG |
| siRNA 575 | 575 | CCCCAGUCCUAUCACUGCA | 2626 | UGCAGUGAUAGGACUGGGG |
| siRNA 576 | 576 | CCCAGUCCUAUCACUGCAG | 2627 | CUGCAGUGAUAGGACUGGG |
| siRNA 577 | 577 | CCAGUCCUAUCACUGCAGC | 2628 | GCUGCAGUGAUAGGACUGG |
| siRNA 578 | 578 | CAGUCCUAUCACUGCAGCU | 2629 | AGCUGCAGUGAUAGGACUG |
| siRNA 579 | 579 | AGUCCUAUCACUGCAGCUG | 2630 | CAGCUGCAGUGAUAGGACU |
| siRNA 580 | 580 | GUCCUAUCACUGCAGCUGC | 2631 | GCAGCUGCAGUGAUAGGAC |
| siRNA 581 | 581 | UCCUAUCACUGCAGCUGCC | 2632 | GGCAGCUGCAGUGAUAGGA |
| siRNA 582 | 582 | CCUAUCACUGCAGCUGCCC | 2633 | GGGCAGCUGCAGUGAUAGG |
| siRNA 583 | 583 | CUAUCACUGCAGCUGCCCC | 2634 | GGGGCAGCUGCAGUGAUAG |
| siRNA 584 | 584 | UAUCACUGCAGCUGCCCCC | 2635 | GGGGGCAGCUGCAGUGAUA |
| siRNA 585 | 585 | AUCACUGCAGCUGCCCCCG | 2636 | CGGGGGCAGCUGCAGUGAU |
| siRNA 586 | 586 | UCACUGCAGCUGCCCCCGG | 2637 | CCGGGGGCAGCUGCAGUGA |
| siRNA 587 | 587 | CACUGCAGCUGCCCCCGGG | 2638 | CCCGGGGGCAGCUGCAGUG |
| siRNA 588 | 588 | ACUGCAGCUGCCCCCGGGC | 2639 | GCCCGGGGGCAGCUGCAGU |
| siRNA 589 | 589 | CUGCAGCUGCCCCCGGGCC | 2640 | GGCCCGGGGGCAGCUGCAG |
| siRNA 590 | 590 | UGCAGCUGCCCCCGGGCCU | 2641 | AGGCCCGGGGGCAGCUGCA |
| siRNA 591 | 591 | GCAGCUGCCCCCGGGCCUU | 2642 | AAGGCCCGGGGGCAGCUGC |
| siRNA 592 | 592 | CAGCUGCCCCCGGGCCUUC | 2643 | GAAGGCCCGGGGGCAGCUG |
| siRNA 593 | 593 | AGCUGCCCCCGGGCCUUCA | 2644 | UGAAGGCCCGGGGGCAGCU |
| siRNA 594 | 594 | GCUGCCCCCGGGCCUUCAC | 2645 | GUGAAGGCCCGGGGGCAGC |
| siRNA 595 | 595 | CUGCCCCCGGGCCUUCACC | 2646 | GGUGAAGGCCCGGGGGCAG |
| siRNA 596 | 596 | UGCCCCCGGGCCUUCACCG | 2647 | CGGUGAAGGCCCGGGGGCA |
| siRNA 597 | 597 | GCCCCCGGGCCUUCACCGG | 2648 | CCGGUGAAGGCCCGGGGGC |
| siRNA 598 | 598 | CCCCCGGGCCUUCACCGGC | 2649 | GCCGGUGAAGGCCCGGGGG |
| siRNA 599 | 599 | CCCCGGGCCUUCACCGGCA | 2650 | UGCCGGUGAAGGCCCGGGG |
| siRNA 600 | 600 | CCCGGGCCUUCACCGGCAA | 2651 | UUGCCGGUGAAGGCCCGGG |
| siRNA 601 | 601 | CCGGGCCUUCACCGGCAAG | 2652 | CUUGCCGGUGAAGGCCCGG |
| siRNA 602 | 602 | CGGGCCUUCACCGGCAAGG | 2653 | CCUUGCCGGUGAAGGCCCG |
| siRNA 603 | 603 | GGGCCUUCACCGGCAAGGA | 2654 | UCCUUGCCGGUGAAGGCCC |
| siRNA 604 | 604 | GGCCUUCACCGGCAAGGAC | 2655 | GUCCUUGCCGGUGAAGGCC |
| siRNA 605 | 605 | GCCUUCACCGGCAAGGACU | 2656 | AGUCCUUGCCGGUGAAGGC |
| siRNA 606 | 606 | CCUUCACCGGCAAGGACUG | 2657 | CAGUCCUUGCCGGUGAAGG |
| siRNA 607 | 607 | CUUCACCGGCAAGGACUGC | 2658 | GCAGUCCUUGCCGGUGAAG |
| siRNA 608 | 608 | UUCACCGGCAAGGACUGCG | 2659 | CGCAGUCCUUGCCGGUGAA |
| siRNA 609 | 609 | UCACCGGCAAGGACUGCGG | 2660 | CCGCAGUCCUUGCCGGUGA |
| siRNA 610 | 610 | CACCGGCAAGGACUGCGGC | 2661 | GCCGCAGUCCUUGCCGGUG |
| siRNA 611 | 611 | ACCGGCAAGGACUGCGGCA | 2662 | UGCCGCAGUCCUUGCCGGU |
| siRNA 612 | 612 | CCGGCAAGGACUGCGGCAC | 2663 | GUGCCGCAGUCCUUGCCGG |
| siRNA 613 | 613 | CGGCAAGGACUGCGGCACA | 2664 | UGUGCCGCAGUCCUUGCCG |
| siRNA 614 | 614 | GGCAAGGACUGCGGCACAG | 2665 | CUGUGCCGCAGUCCUUGCC |
| siRNA 615 | 615 | GCAAGGACUGCGGCACAGA | 2666 | UCUGUGCCGCAGUCCUUGC |
| siRNA 616 | 616 | CAAGGACUGCGGCACAGAG | 2667 | CUCUGUGCCGCAGUCCUUG |
| siRNA 617 | 617 | AAGGACUGCGGCACAGAGA | 2668 | UCUCUGUGCCGCAGUCCUU |
| siRNA 618 | 618 | AGGACUGCGGCACAGAGAA | 2669 | UUCUCUGUGCCGCAGUCCU |
| siRNA 619 | 619 | GGACUGCGGCACAGAGAAA | 2670 | UUUCUCUGUGCCGCAGUCC |
| siRNA 620 | 620 | GACUGCGGCACAGAGAAAU | 2671 | AUUUCUCUGUGCCGCAGUC |
| siRNA 621 | 621 | ACUGCGGCACAGAGAAAUG | 2672 | CAUUUCUCUGUGCCGCAGU |
| siRNA 622 | 622 | CUGCGGCACAGAGAAAUGC | 2673 | GCAUUUCUCUGUGCCGCAG |
| siRNA 623 | 623 | UGCGGCACAGAGAAAUGCU | 2674 | AGCAUUUCUCUGUGCCGCA |
| siRNA 624 | 624 | GCGGCACAGAGAAAUGCUU | 2675 | AAGCAUUUCUCUGUGCCGC |
| siRNA 625 | 625 | CGGCACAGAGAAAUGCUUU | 2676 | AAAGCAUUUCUCUGUGCCG |
| siRNA 626 | 626 | GGCACAGAGAAAUGCUUUG | 2677 | CAAAGCAUUUCUCUGUGCC |
| siRNA 627 | 627 | GCACAGAGAAAUGCUUUGA | 2678 | UCAAAGCAUUUCUCUGUGC |
| siRNA 628 | 628 | CACAGAGAAAUGCUUUGAU | 2679 | AUCAAAGCAUUUCUCUGUG |
| siRNA 629 | 629 | ACAGAGAAAUGCUUUGAUG | 2680 | CAUCAAAGCAUUUCUCUGU |
| siRNA 630 | 630 | CAGAGAAAUGCUUUGAUGA | 2681 | UCAUCAAAGCAUUUCUCUG |
| siRNA 631 | 631 | AGAGAAAUGCUUUGAUGAG | 2682 | CUCAUCAAAGCAUUUCUCU |
| siRNA 632 | 632 | GAGAAAUGCUUUGAUGAGA | 2683 | UCUCAUCAAAGCAUUUCUC |
| siRNA 633 | 633 | AGAAAUGCUUUGAUGAGAC | 2684 | GUCUCAUCAAAGCAUUUCU |
| siRNA 634 | 634 | GAAAUGCUUUGAUGAGACC | 2685 | GGUCUCAUCAAAGCAUUUC |
| siRNA 635 | 635 | AAAUGCUUUGAUGAGACCC | 2686 | GGGUCUCAUCAAAGCAUUU |
| siRNA 636 | 636 | AAUGCUUUGAUGAGACCCG | 2687 | CGGGUCUCAUCAAAGCAUU |
| siRNA 637 | 637 | AUGCUUUGAUGAGACCCGC | 2688 | GCGGGUCUCAUCAAAGCAU |
| siRNA 638 | 638 | UGCUUUGAUGAGACCCGCU | 2689 | AGCGGGUCUCAUCAAAGCA |
| siRNA 639 | 639 | GCUUUGAUGAGACCCGCUA | 2690 | UAGCGGGUCUCAUCAAAGC |
| siRNA 640 | 640 | CUUUGAUGAGACCCGCUAC | 2691 | GUAGCGGGUCUCAUCAAAG |
| siRNA 641 | 641 | UUUGAUGAGACCCGCUACG | 2692 | CGUAGCGGGUCUCAUCAAA |
| siRNA 642 | 642 | UUGAUGAGACCCGCUACGA | 2693 | UCGUAGCGGGUCUCAUCAA |
| siRNA 643 | 643 | UGAUGAGACCCGCUACGAG | 2694 | CUCGUAGCGGGUCUCAUCA |
| siRNA 644 | 644 | GAUGAGACCCGCUACGAGU | 2695 | ACUCGUAGCGGGUCUCAUC |
| siRNA 645 | 645 | AUGAGACCCGCUACGAGUA | 2696 | UACUCGUAGCGGGUCUCAU |
| siRNA 646 | 646 | UGAGACCCGCUACGAGUAC | 2697 | GUACUCGUAGCGGGUCUCA |
| siRNA 647 | 647 | GAGACCCGCUACGAGUACC | 2698 | GGUACUCGUAGCGGGUCUC |
| siRNA 648 | 648 | AGACCCGCUACGAGUACCU | 2699 | AGGUACUCGUAGCGGGUCU |
| siRNA 649 | 649 | GACCCGCUACGAGUACCUG | 2700 | CAGGUACUCGUAGCGGGUC |
| siRNA 650 | 650 | ACCCGCUACGAGUACCUGG | 2701 | CCAGGUACUCGUAGCGGGU |
| siRNA 651 | 651 | CCCGCUACGAGUACCUGGA | 2702 | UCCAGGUACUCGUAGCGGG |
| siRNA 652 | 652 | CCGCUACGAGUACCUGGAG | 2703 | CUCCAGGUACUCGUAGCGG |
| siRNA 653 | 653 | CGCUACGAGUACCUGGAGG | 2704 | CCUCCAGGUACUCGUAGCG |
| siRNA 654 | 654 | GCUACGAGUACCUGGAGGG | 2705 | CCCUCCAGGUACUCGUAGC |
| siRNA 655 | 655 | CUACGAGUACCUGGAGGGG | 2706 | CCCCUCCAGGUACUCGUAG |
| siRNA 656 | 656 | UACGAGUACCUGGAGGGGG | 2707 | CCCCCUCCAGGUACUCGUA |
| siRNA 657 | 657 | ACGAGUACCUGGAGGGGGG | 2708 | CCCCCCUCCAGGUACUCGU |
| siRNA 658 | 658 | CGAGUACCUGGAGGGGGGC | 2709 | GCCCCCCUCCAGGUACUCG |
| siRNA 659 | 659 | GAGUACCUGGAGGGGGGCG | 2710 | CGCCCCCCUCCAGGUACUC |
| siRNA 660 | 660 | AGUACCUGGAGGGGGGCGA | 2711 | UCGCCCCCCUCCAGGUACU |
| siRNA 661 | 661 | GUACCUGGAGGGGGGCGAC | 2712 | GUCGCCCCCCUCCAGGUAC |
| siRNA 662 | 662 | UACCUGGAGGGGGGCGACC | 2713 | GGUCGCCCCCCUCCAGGUA |
| siRNA 663 | 663 | ACCUGGAGGGGGGCGACCG | 2714 | CGGUCGCCCCCCUCCAGGU |
| siRNA 664 | 664 | CCUGGAGGGGGGCGACCGC | 2715 | GCGGUCGCCCCCCUCCAGG |
| siRNA 665 | 665 | CUGGAGGGGGGCGACCGCU | 2716 | AGCGGUCGCCCCCCUCCAG |
| siRNA 666 | 666 | UGGAGGGGGGCGACCGCUG | 2717 | CAGCGGUCGCCCCCCUCCA |
| siRNA 667 | 667 | GGAGGGGGGCGACCGCUGG | 2718 | CCAGCGGUCGCCCCCCUCC |
| siRNA 668 | 668 | GAGGGGGGCGACCGCUGGG | 2719 | CCCAGCGGUCGCCCCCCUC |
| siRNA 669 | 669 | AGGGGGGCGACCGCUGGGC | 2720 | GCCCAGCGGUCGCCCCCCU |
| siRNA 670 | 670 | GGGGGGCGACCGCUGGGCC | 2721 | GGCCCAGCGGUCGCCCCCC |
| siRNA 671 | 671 | GGGGGCGACCGCUGGGCCC | 2722 | GGGCCCAGCGGUCGCCCCC |
| siRNA 672 | 672 | GGGGCGACCGCUGGGCCCG | 2723 | CGGGCCCAGCGGUCGCCCC |
| siRNA 673 | 673 | GGGCGACCGCUGGGCCCGC | 2724 | GCGGGCCCAGCGGUCGCCC |
| siRNA 674 | 674 | GGCGACCGCUGGGCCCGCG | 2725 | CGCGGGCCCAGCGGUCGCC |
| siRNA 675 | 675 | GCGACCGCUGGGCCCGCGU | 2726 | ACGCGGGCCCAGCGGUCGC |
| siRNA 676 | 676 | CGACCGCUGGGCCCGCGUG | 2727 | CACGCGGGCCCAGCGGUCG |
| siRNA 677 | 677 | GACCGCUGGGCCCGCGUGC | 2728 | GCACGCGGGCCCAGCGGUC |
| siRNA 678 | 678 | ACCGCUGGGCCCGCGUGCG | 2729 | CGCACGCGGGCCCAGCGGU |
| siRNA 679 | 679 | CCGCUGGGCCCGCGUGCGC | 2730 | GCGCACGCGGGCCCAGCGG |
| siRNA 680 | 680 | CGCUGGGCCCGCGUGCGCC | 2731 | GGCGCACGCGGGCCCAGCG |
| siRNA 681 | 681 | GCUGGGCCCGCGUGCGCCA | 2732 | UGGCGCACGCGGGCCCAGC |
| siRNA 682 | 682 | CUGGGCCCGCGUGCGCCAG | 2733 | CUGGCGCACGCGGGCCCAG |
| siRNA 683 | 683 | UGGGCCCGCGUGCGCCAGG | 2734 | CCUGGCGCACGCGGGCCCA |
| siRNA 684 | 684 | GGGCCCGCGUGCGCCAGGG | 2735 | CCCUGGCGCACGCGGGCCC |
| siRNA 685 | 685 | GGCCCGCGUGCGCCAGGGC | 2736 | GCCCUGGCGCACGCGGGCC |
| siRNA 686 | 686 | GCCCGCGUGCGCCAGGGCC | 2737 | GGCCCUGGCGCACGCGGGC |
| siRNA 687 | 687 | CCCGCGUGCGCCAGGGCCA | 2738 | UGGCCCUGGCGCACGCGGG |
| siRNA 688 | 688 | CCGCGUGCGCCAGGGCCAC | 2739 | GUGGCCCUGGCGCACGCGG |
| siRNA 689 | 689 | CGCGUGCGCCAGGGCCACG | 2740 | CGUGGCCCUGGCGCACGCG |
| siRNA 690 | 690 | GCGUGCGCCAGGGCCACGU | 2741 | ACGUGGCCCUGGCGCACGC |
| siRNA 691 | 691 | CGUGCGCCAGGGCCACGUG | 2742 | CACGUGGCCCUGGCGCACG |
| siRNA 692 | 692 | GUGCGCCAGGGCCACGUGG | 2743 | CCACGUGGCCCUGGCGCAC |
| siRNA 693 | 693 | UGCGCCAGGGCCACGUGGA | 2744 | UCCACGUGGCCCUGGCGCA |
| siRNA 694 | 694 | GCGCCAGGGCCACGUGGAA | 2745 | UUCCACGUGGCCCUGGCGC |
| siRNA 695 | 695 | CGCCAGGGCCACGUGGAAC | 2746 | GUUCCACGUGGCCCUGGCG |
| siRNA 696 | 696 | GCCAGGGCCACGUGGAACA | 2747 | UGUUCCACGUGGCCCUGGC |
| siRNA 697 | 697 | CCAGGGCCACGUGGAACAG | 2748 | CUGUUCCACGUGGCCCUGG |
| siRNA 698 | 698 | CAGGGCCACGUGGAACAGU | 2749 | ACUGUUCCACGUGGCCCUG |
| siRNA 699 | 699 | AGGGCCACGUGGAACAGUG | 2750 | CACUGUUCCACGUGGCCCU |
| siRNA 700 | 700 | GGGCCACGUGGAACAGUGC | 2751 | GCACUGUUCCACGUGGCCC |
| siRNA 701 | 701 | GGCCACGUGGAACAGUGCG | 2752 | CGCACUGUUCCACGUGGCC |
| siRNA 702 | 702 | GCCACGUGGAACAGUGCGA | 2753 | UCGCACUGUUCCACGUGGC |
| siRNA 703 | 703 | CCACGUGGAACAGUGCGAG | 2754 | CUCGCACUGUUCCACGUGG |
| siRNA 704 | 704 | CACGUGGAACAGUGCGAGU | 2755 | ACUCGCACUGUUCCACGUG |
| siRNA 705 | 705 | ACGUGGAACAGUGCGAGUG | 2756 | CACUCGCACUGUUCCACGU |
| siRNA 706 | 706 | CGUGGAACAGUGCGAGUGC | 2757 | GCACUCGCACUGUUCCACG |
| siRNA 707 | 707 | GUGGAACAGUGCGAGUGCU | 2758 | AGCACUCGCACUGUUCCAC |
| siRNA 708 | 708 | UGGAACAGUGCGAGUGCUU | 2759 | AAGCACUCGCACUGUUCCA |
| siRNA 709 | 709 | GGAACAGUGCGAGUGCUUC | 2760 | GAAGCACUCGCACUGUUCC |
| siRNA 710 | 710 | GAACAGUGCGAGUGCUUCG | 2761 | CGAAGCACUCGCACUGUUC |
| siRNA 711 | 711 | AACAGUGCGAGUGCUUCGG | 2762 | CCGAAGCACUCGCACUGUU |
| siRNA 712 | 712 | ACAGUGCGAGUGCUUCGGG | 2763 | CCCGAAGCACUCGCACUGU |
| siRNA 713 | 713 | CAGUGCGAGUGCUUCGGGG | 2764 | CCCCGAAGCACUCGCACUG |
| siRNA 714 | 714 | AGUGCGAGUGCUUCGGGGG | 2765 | CCCCCGAAGCACUCGCACU |
| siRNA 715 | 715 | GUGCGAGUGCUUCGGGGGC | 2766 | GCCCCCGAAGCACUCGCAC |
| siRNA 716 | 716 | UGCGAGUGCUUCGGGGGCC | 2767 | GGCCCCCGAAGCACUCGCA |
| siRNA 717 | 717 | GCGAGUGCUUCGGGGGCCG | 2768 | CGGCCCCCGAAGCACUCGC |
| siRNA 718 | 718 | CGAGUGCUUCGGGGGCCGG | 2769 | CCGGCCCCCGAAGCACUCG |
| SiRNA 719 | 719 | GAGUGCUUCGGGGGCCGGA | 2770 | UCCGGCCCCCGAAGCACUC |
| siRNA 720 | 720 | AGUGCUUCGGGGGCCGGAC | 2771 | GUCCGGCCCCCGAAGCACU |
| siRNA 721 | 721 | GUGCUUCGGGGGCCGGACC | 2772 | GGUCCGGCCCCCGAAGCAC |
| siRNA 722 | 722 | UGCUUCGGGGGCCGGACCU | 2773 | AGGUCCGGCCCCCGAAGCA |
| siRNA 723 | 723 | GCUUCGGGGGCCGGACCUG | 2774 | CAGGUCCGGCCCCCGAAGC |
| siRNA 724 | 724 | CUUCGGGGGCCGGACCUGG | 2775 | CCAGGUCCGGCCCCCGAAG |
| siRNA 725 | 725 | UUCGGGGGCCGGACCUGGU | 2776 | ACCAGGUCCGGCCCCCGAA |
| siRNA 726 | 726 | UCGGGGGCCGGACCUGGUG | 2777 | CACCAGGUCCGGCCCCCGA |
| siRNA 727 | 727 | CGGGGGCCGGACCUGGUGC | 2778 | GCACCAGGUCCGGCCCCCG |
| siRNA 728 | 728 | GGGGGCCGGACCUGGUGCG | 2779 | CGCACCAGGUCCGGCCCCC |
| siRNA 729 | 729 | GGGGCCGGACCUGGUGCGA | 2780 | UCGCACCAGGUCCGGCCCC |
| siRNA 730 | 730 | GGGCCGGACCUGGUGCGAA | 2781 | UUCGCACCAGGUCCGGCCC |
| siRNA 731 | 731 | GGCCGGACCUGGUGCGAAG | 2782 | CUUCGCACCAGGUCCGGCC |
| siRNA 732 | 732 | GCCGGACCUGGUGCGAAGG | 2783 | CCUUCGCACCAGGUCCGGC |
| siRNA 733 | 733 | CCGGACCUGGUGCGAAGGC | 2784 | GCCUUCGCACCAGGUCCGG |
| siRNA 734 | 734 | CGGACCUGGUGCGAAGGCA | 2785 | UGCCUUCGCACCAGGUCCG |
| siRNA 735 | 735 | GGACCUGGUGCGAAGGCAC | 2786 | GUGCCUUCGCACCAGGUCC |
| siRNA 736 | 736 | GACCUGGUGCGAAGGCACC | 2787 | GGUGCCUUCGCACCAGGUC |
| siRNA 737 | 737 | ACCUGGUGCGAAGGCACCC | 2788 | GGGUGCCUUCGCACCAGGU |
| siRNA 738 | 738 | CCUGGUGCGAAGGCACCCG | 2789 | CGGGUGCCUUCGCACCAGG |
| siRNA 739 | 739 | CUGGUGCGAAGGCACCCGA | 2790 | UCGGGUGCCUUCGCACCAG |
| siRNA 740 | 740 | UGGUGCGAAGGCACCCGAC | 2791 | GUCGGGUGCCUUCGCACCA |
| siRNA 741 | 741 | GGUGCGAAGGCACCCGACA | 2792 | UGUCGGGUGCCUUCGCACC |
| siRNA 742 | 742 | GUGCGAAGGCACCCGACAU | 2793 | AUGUCGGGUGCCUUCGCAC |
| siRNA 743 | 743 | UGCGAAGGCACCCGACAUA | 2794 | UAUGUCGGGUGCCUUCGCA |
| siRNA 744 | 744 | GCGAAGGCACCCGACAUAC | 2795 | GUAUGUCGGGUGCCUUCGC |
| siRNA 745 | 745 | CGAAGGCACCCGACAUACA | 2796 | UGUAUGUCGGGUGCCUUCG |
| siRNA 746 | 746 | GAAGGCACCCGACAUACAG | 2797 | CUGUAUGUCGGGUGCCUUC |
| siRNA 747 | 747 | AAGGCACCCGACAUACAGC | 2798 | GCUGUAUGUCGGGUGCCUU |
| siRNA 748 | 748 | AGGCACCCGACAUACAGCU | 2799 | AGCUGUAUGUCGGGUGCCU |
| siRNA 749 | 749 | GGCACCCGACAUACAGCUU | 2800 | AAGCUGUAUGUCGGGUGCC |
| siRNA 750 | 750 | GCACCCGACAUACAGCUUG | 2801 | CAAGCUGUAUGUCGGGUGC |
| siRNA 751 | 751 | CACCCGACAUACAGCUUGU | 2802 | ACAAGCUGUAUGUCGGGUG |
| siRNA 752 | 752 | ACCCGACAUACAGCUUGUC | 2803 | GACAAGCUGUAUGUCGGGU |
| siRNA 753 | 753 | CCCGACAUACAGCUUGUCU | 2804 | AGACAAGCUGUAUGUCGGG |
| siRNA 754 | 754 | CCGACAUACAGCUUGUCUG | 2805 | CAGACAAGCUGUAUGUCGG |
| siRNA 755 | 755 | CGACAUACAGCUUGUCUGA | 2806 | UCAGACAAGCUGUAUGUCG |
| siRNA 756 | 756 | GACAUACAGCUUGUCUGAG | 2807 | CUCAGACAAGCUGUAUGUC |
| siRNA 757 | 757 | ACAUACAGCUUGUCUGAGC | 2808 | GCUCAGACAAGCUGUAUGU |
| siRNA 758 | 758 | CAUACAGCUUGUCUGAGCA | 2809 | UGCUCAGACAAGCUGUAUG |
| siRNA 759 | 759 | AUACAGCUUGUCUGAGCAG | 2810 | CUGCUCAGACAAGCUGUAU |
| siRNA 760 | 760 | UACAGCUUGUCUGAGCAGC | 2811 | GCUGCUCAGACAAGCUGUA |
| siRNA 761 | 761 | ACAGCUUGUCUGAGCAGCC | 2812 | GGCUGCUCAGACAAGCUGU |
| siRNA 762 | 762 | CAGCUUGUCUGAGCAGCCC | 2813 | GGGCUGCUCAGACAAGCUG |
| siRNA 763 | 763 | AGCUUGUCUGAGCAGCCCU | 2814 | AGGGCUGCUCAGACAAGCU |
| siRNA 764 | 764 | GCUUGUCUGAGCAGCCCUU | 2815 | AAGGGCUGCUCAGACAAGC |
| siRNA 765 | 765 | CUUGUCUGAGCAGCCCUUG | 2816 | CAAGGGCUGCUCAGACAAG |
| siRNA 766 | 766 | UUGUCUGAGCAGCCCUUGC | 2817 | GCAAGGGCUGCUCAGACAA |
| siRNA 767 | 767 | UGUCUGAGCAGCCCUUGCC | 2818 | GGCAAGGGCUGCUCAGACA |
| siRNA 768 | 768 | GUCUGAGCAGCCCUUGCCU | 2819 | AGGCAAGGGCUGCUCAGAC |
| siRNA 769 | 769 | UCUGAGCAGCCCUUGCCUG | 2820 | CAGGCAAGGGCUGCUCAGA |
| siRNA 770 | 770 | CUGAGCAGCCCUUGCCUGA | 2821 | UCAGGCAAGGGCUGCUCAG |
| siRNA 771 | 771 | UGAGCAGCCCUUGCCUGAA | 2822 | UUCAGGCAAGGGCUGCUCA |
| siRNA 772 | 772 | GAGCAGCCCUUGCCUGAAC | 2823 | GUUCAGGCAAGGGCUGCUC |
| siRNA 773 | 773 | AGCAGCCCUUGCCUGAACG | 2824 | CGUUCAGGCAAGGGCUGCU |
| siRNA 774 | 774 | GCAGCCCUUGCCUGAACGG | 2825 | CCGUUCAGGCAAGGGCUGC |
| siRNA 775 | 775 | CAGCCCUUGCCUGAACGGG | 2826 | CCCGUUCAGGCAAGGGCUG |
| siRNA 776 | 776 | AGCCCUUGCCUGAACGGGG | 2827 | CCCCGUUCAGGCAAGGGCU |
| siRNA 777 | 777 | GCCCUUGCCUGAACGGGGG | 2828 | CCCCCGUUCAGGCAAGGGC |
| siRNA 778 | 778 | CCCUUGCCUGAACGGGGGC | 2829 | GCCCCCGUUCAGGCAAGGG |
| siRNA 779 | 779 | CCUUGCCUGAACGGGGGCA | 2830 | UGCCCCCGUUCAGGCAAGG |
| siRNA 780 | 780 | CUUGCCUGAACGGGGGCAC | 2831 | GUGCCCCCGUUCAGGCAAG |
| siRNA 781 | 781 | UUGCCUGAACGGGGGCACC | 2832 | GGUGCCCCCGUUCAGGCAA |
| siRNA 782 | 782 | UGCCUGAACGGGGGCACCU | 2833 | AGGUGCCCCCGUUCAGGCA |
| siRNA 783 | 783 | GCCUGAACGGGGGCACCUG | 2834 | CAGGUGCCCCCGUUCAGGC |
| siRNA 784 | 784 | CCUGAACGGGGGCACCUGC | 2835 | GCAGGUGCCCCCGUUCAGG |
| siRNA 785 | 785 | CUGAACGGGGGCACCUGCC | 2836 | GGCAGGUGCCCCCGUUCAG |
| siRNA 786 | 786 | UGAACGGGGGCACCUGCCA | 2837 | UGGCAGGUGCCCCCGUUCA |
| siRNA 787 | 787 | GAACGGGGGCACCUGCCAC | 2838 | GUGGCAGGUGCCCCCGUUC |
| siRNA 788 | 788 | AACGGGGGCACCUGCCACC | 2839 | GGUGGCAGGUGCCCCCGUU |
| siRNA 789 | 789 | ACGGGGGCACCUGCCACCU | 2840 | AGGUGGCAGGUGCCCCCGU |
| siRNA 790 | 790 | CGGGGGCACCUGCCACCUG | 2841 | CAGGUGGCAGGUGCCCCCG |
| siRNA 791 | 791 | GGGGGCACCUGCCACCUGA | 2842 | UCAGGUGGCAGGUGCCCCC |
| siRNA 792 | 792 | GGGGCACCUGCCACCUGAU | 2843 | AUCAGGUGGCAGGUGCCCC |
| siRNA 793 | 793 | GGGCACCUGCCACCUGAUC | 2844 | GAUCAGGUGGCAGGUGCCC |
| siRNA 794 | 794 | GGCACCUGCCACCUGAUCG | 2845 | CGAUCAGGUGGCAGGUGCC |
| siRNA 795 | 795 | GCACCUGCCACCUGAUCGU | 2846 | ACGAUCAGGUGGCAGGUGC |
| siRNA 796 | 796 | CACCUGCCACCUGAUCGUG | 2847 | CACGAUCAGGUGGCAGGUG |
| siRNA 797 | 797 | ACCUGCCACCUGAUCGUGG | 2848 | CCACGAUCAGGUGGCAGGU |
| siRNA 798 | 798 | CCUGCCACCUGAUCGUGGC | 2849 | GCCACGAUCAGGUGGCAGG |
| siRNA 799 | 799 | CUGCCACCUGAUCGUGGCC | 2850 | GGCCACGAUCAGGUGGCAG |
| siRNA 800 | 800 | UGCCACCUGAUCGUGGCCA | 2851 | UGGCCACGAUCAGGUGGCA |
| siRNA 801 | 801 | GCCACCUGAUCGUGGCCAC | 2852 | GUGGCCACGAUCAGGUGGC |
| siRNA 802 | 802 | CCACCUGAUCGUGGCCACC | 2853 | GGUGGCCACGAUCAGGUGG |
| siRNA 803 | 803 | CACCUGAUCGUGGCCACCG | 2854 | CGGUGGCCACGAUCAGGUG |
| siRNA 804 | 804 | ACCUGAUCGUGGCCACCGG | 2855 | CCGGUGGCCACGAUCAGGU |
| siRNA 805 | 805 | CCUGAUCGUGGCCACCGGG | 2856 | CCCGGUGGCCACGAUCAGG |
| siRNA 806 | 806 | CUGAUCGUGGCCACCGGGA | 2857 | UCCCGGUGGCCACGAUCAG |
| siRNA 807 | 807 | UGAUCGUGGCCACCGGGAC | 2858 | GUCCCGGUGGCCACGAUCA |
| siRNA 808 | 808 | GAUCGUGGCCACCGGGACC | 2859 | GGUCCCGGUGGCCACGAUC |
| siRNA 809 | 809 | AUCGUGGCCACCGGGACCA | 2860 | UGGUCCCGGUGGCCACGAU |
| siRNA 810 | 810 | UCGUGGCCACCGGGACCAC | 2861 | GUGGUCCCGGUGGCCACGA |
| siRNA 811 | 811 | CGUGGCCACCGGGACCACC | 2862 | GGUGGUCCCGGUGGCCACG |
| siRNA 812 | 812 | GUGGCCACCGGGACCACCG | 2863 | CGGUGGUCCCGGUGGCCAC |
| siRNA 813 | 813 | UGGCCACCGGGACCACCGU | 2864 | ACGGUGGUCCCGGUGGCCA |
| siRNA 814 | 814 | GGCCACCGGGACCACCGUG | 2865 | CACGGUGGUCCCGGUGGCC |
| siRNA 815 | 815 | GCCACCGGGACCACCGUGU | 2866 | ACACGGUGGUCCCGGUGGC |
| siRNA 816 | 816 | CCACCGGGACCACCGUGUG | 2867 | CACACGGUGGUCCCGGUGG |
| siRNA 817 | 817 | CACCGGGACCACCGUGUGU | 2868 | ACACACGGUGGUCCCGGUG |
| siRNA 818 | 818 | ACCGGGACCACCGUGUGUG | 2869 | CACACACGGUGGUCCCGGU |
| siRNA 819 | 819 | CCGGGACCACCGUGUGUGC | 2870 | GCACACACGGUGGUCCCGG |
| siRNA 820 | 820 | CGGGACCACCGUGUGUGCC | 2871 | GGCACACACGGUGGUCCCG |
| siRNA 821 | 821 | GGGACCACCGUGUGUGCCU | 2872 | AGGCACACACGGUGGUCCC |
| siRNA 822 | 822 | GGACCACCGUGUGUGCCUG | 2873 | CAGGCACACACGGUGGUCC |
| siRNA 823 | 823 | GACCACCGUGUGUGCCUGC | 2874 | GCAGGCACACACGGUGGUC |
| siRNA 824 | 824 | ACCACCGUGUGUGCCUGCC | 2875 | GGCAGGCACACACGGUGGU |
| siRNA 825 | 825 | CCACCGUGUGUGCCUGCCC | 2876 | GGGCAGGCACACACGGUGG |
| siRNA 826 | 826 | CACCGUGUGUGCCUGCCCA | 2877 | UGGGCAGGCACACACGGUG |
| siRNA 827 | 827 | ACCGUGUGUGCCUGCCCAC | 2878 | GUGGGCAGGCACACACGGU |
| siRNA 828 | 828 | CCGUGUGUGCCUGCCCACC | 2879 | GGUGGGCAGGCACACACGG |
| siRNA 829 | 829 | CGUGUGUGCCUGCCCACCA | 2880 | UGGUGGGCAGGCACACACG |
| siRNA 830 | 830 | GUGUGUGCCUGCCCACCAG | 2881 | CUGGUGGGCAGGCACACAC |
| siRNA 831 | 831 | UGUGUGCCUGCCCACCAGG | 2882 | CCUGGUGGGCAGGCACACA |
| siRNA 832 | 832 | GUGUGCCUGCCCACCAGGC | 2883 | GCCUGGUGGGCAGGCACAC |
| siRNA 833 | 833 | UGUGCCUGCCCACCAGGCU | 2884 | AGCCUGGUGGGCAGGCACA |
| siRNA 834 | 834 | GUGCCUGCCCACCAGGCUU | 2885 | AAGCCUGGUGGGCAGGCAC |
| siRNA 835 | 835 | UGCCUGCCCACCAGGCUUC | 2886 | GAAGCCUGGUGGGCAGGCA |
| siRNA 836 | 836 | GCCUGCCCACCAGGCUUCG | 2887 | CGAAGCCUGGUGGGCAGGC |
| siRNA 837 | 837 | CCUGCCCACCAGGCUUCGC | 2888 | GCGAAGCCUGGUGGGCAGG |
| siRNA 838 | 838 | CUGCCCACCAGGCUUCGCU | 2889 | AGCGAAGCCUGGUGGGCAG |
| siRNA 839 | 839 | UGCCCACCAGGCUUCGCUG | 2890 | CAGCGAAGCCUGGUGGGCA |
| siRNA 840 | 840 | GCCCACCAGGCUUCGCUGG | 2891 | CCAGCGAAGCCUGGUGGGC |
| siRNA 841 | 841 | CCCACCAGGCUUCGCUGGA | 2892 | UCCAGCGAAGCCUGGUGGG |
| siRNA 842 | 842 | CCACCAGGCUUCGCUGGAC | 2893 | GUCCAGCGAAGCCUGGUGG |
| siRNA 843 | 843 | CACCAGGCUUCGCUGGACG | 2894 | CGUCCAGCGAAGCCUGGUG |
| siRNA 844 | 844 | ACCAGGCUUCGCUGGACGG | 2895 | CCGUCCAGCGAAGCCUGGU |
| siRNA 845 | 845 | CCAGGCUUCGCUGGACGGC | 2896 | GCCGUCCAGCGAAGCCUGG |
| siRNA 846 | 846 | CAGGCUUCGCUGGACGGCU | 2897 | AGCCGUCCAGCGAAGCCUG |
| siRNA 847 | 847 | AGGCUUCGCUGGACGGCUC | 2898 | GAGCCGUCCAGCGAAGCCU |
| siRNA 848 | 848 | GGCUUCGCUGGACGGCUCU | 2899 | AGAGCCGUCCAGCGAAGCC |
| siRNA 849 | 849 | GCUUCGCUGGACGGCUCUG | 2900 | CAGAGCCGUCCAGCGAAGC |
| siRNA 850 | 850 | CUUCGCUGGACGGCUCUGC | 2901 | GCAGAGCCGUCCAGCGAAG |
| siRNA 851 | 851 | UUCGCUGGACGGCUCUGCA | 2902 | UGCAGAGCCGUCCAGCGAA |
| siRNA 852 | 852 | UCGCUGGACGGCUCUGCAA | 2903 | UUGCAGAGCCGUCCAGCGA |
| siRNA 853 | 853 | CGCUGGACGGCUCUGCAAC | 2904 | GUUGCAGAGCCGUCCAGCG |
| siRNA 854 | 854 | GCUGGACGGCUCUGCAACA | 2905 | UGUUGCAGAGCCGUCCAGC |
| siRNA 855 | 855 | CUGGACGGCUCUGCAACAU | 2906 | AUGUUGCAGAGCCGUCCAG |
| siRNA 856 | 856 | UGGACGGCUCUGCAACAUC | 2907 | GAUGUUGCAGAGCCGUCCA |
| siRNA 857 | 857 | GGACGGCUCUGCAACAUCG | 2908 | CGAUGUUGCAGAGCCGUCC |
| siRNA 858 | 858 | GACGGCUCUGCAACAUCGA | 2909 | UCGAUGUUGCAGAGCCGUC |
| siRNA 859 | 859 | ACGGCUCUGCAACAUCGAG | 2910 | CUCGAUGUUGCAGAGCCGU |
| siRNA 860 | 860 | CGGCUCUGCAACAUCGAGC | 2911 | GCUCGAUGUUGCAGAGCCG |
| siRNA 861 | 861 | GGCUCUGCAACAUCGAGCC | 2912 | GGCUCGAUGUUGCAGAGCC |
| siRNA 862 | 862 | GCUCUGCAACAUCGAGCCU | 2913 | AGGCUCGAUGUUGCAGAGC |
| siRNA 863 | 863 | CUCUGCAACAUCGAGCCUG | 2914 | CAGGCUCGAUGUUGCAGAG |
| siRNA 864 | 864 | UCUGCAACAUCGAGCCUGA | 2915 | UCAGGCUCGAUGUUGCAGA |
| siRNA 865 | 865 | CUGCAACAUCGAGCCUGAU | 2916 | AUCAGGCUCGAUGUUGCAG |
| siRNA 866 | 866 | UGCAACAUCGAGCCUGAUG | 2917 | CAUCAGGCUCGAUGUUGCA |
| siRNA 867 | 867 | GCAACAUCGAGCCUGAUGA | 2918 | UCAUCAGGCUCGAUGUUGC |
| siRNA 868 | 868 | CAACAUCGAGCCUGAUGAG | 2919 | CUCAUCAGGCUCGAUGUUG |
| siRNA 869 | 869 | AACAUCGAGCCUGAUGAGC | 2920 | GCUCAUCAGGCUCGAUGUU |
| siRNA 870 | 870 | ACAUCGAGCCUGAUGAGCG | 2921 | CGCUCAUCAGGCUCGAUGU |
| siRNA 871 | 871 | CAUCGAGCCUGAUGAGCGC | 2922 | GCGCUCAUCAGGCUCGAUG |
| siRNA 872 | 872 | AUCGAGCCUGAUGAGCGCU | 2923 | AGCGCUCAUCAGGCUCGAU |
| siRNA 873 | 873 | UCGAGCCUGAUGAGCGCUG | 2924 | CAGCGCUCAUCAGGCUCGA |
| siRNA 874 | 874 | CGAGCCUGAUGAGCGCUGC | 2925 | GCAGCGCUCAUCAGGCUCG |
| siRNA 875 | 875 | GAGCCUGAUGAGCGCUGCU | 2926 | AGCAGCGCUCAUCAGGCUC |
| siRNA 876 | 876 | AGCCUGAUGAGCGCUGCUU | 2927 | AAGCAGCGCUCAUCAGGCU |
| siRNA 877 | 877 | GCCUGAUGAGCGCUGCUUC | 2928 | GAAGCAGCGCUCAUCAGGC |
| siRNA 878 | 878 | CCUGAUGAGCGCUGCUUCU | 2929 | AGAAGCAGCGCUCAUCAGG |
| siRNA 879 | 879 | CUGAUGAGCGCUGCUUCUU | 2930 | AAGAAGCAGCGCUCAUCAG |
| siRNA 880 | 880 | UGAUGAGCGCUGCUUCUUG | 2931 | CAAGAAGCAGCGCUCAUCA |
| siRNA 881 | 881 | GAUGAGCGCUGCUUCUUGG | 2932 | CCAAGAAGCAGCGCUCAUC |
| siRNA 882 | 882 | AUGAGCGCUGCUUCUUGGG | 2933 | CCCAAGAAGCAGCGCUCAU |
| siRNA 883 | 883 | UGAGCGCUGCUUCUUGGGG | 2934 | CCCCAAGAAGCAGCGCUCA |
| siRNA 884 | 884 | GAGCGCUGCUUCUUGGGGA | 2935 | UCCCCAAGAAGCAGCGCUC |
| siRNA 885 | 885 | AGCGCUGCUUCUUGGGGAA | 2936 | UUCCCCAAGAAGCAGCGCU |
| siRNA 886 | 886 | GCGCUGCUUCUUGGGGAAC | 2937 | GUUCCCCAAGAAGCAGCGC |
| siRNA 887 | 887 | CGCUGCUUCUUGGGGAACG | 2938 | CGUUCCCCAAGAAGCAGCG |
| siRNA 888 | 888 | GCUGCUUCUUGGGGAACGG | 2939 | CCGUUCCCCAAGAAGCAGC |
| siRNA 889 | 889 | CUGCUUCUUGGGGAACGGC | 2940 | GCCGUUCCCCAAGAAGCAG |
| siRNA 890 | 890 | UGCUUCUUGGGGAACGGCA | 2941 | UGCCGUUCCCCAAGAAGCA |
| siRNA 891 | 891 | GCUUCUUGGGGAACGGCAC | 2942 | GUGCCGUUCCCCAAGAAGC |
| siRNA 892 | 892 | CUUCUUGGGGAACGGCACU | 2943 | AGUGCCGUUCCCCAAGAAG |
| siRNA 893 | 893 | UUCUUGGGGAACGGCACUG | 2944 | CAGUGCCGUUCCCCAAGAA |
| siRNA 894 | 894 | UCUUGGGGAACGGCACUGG | 2945 | CCAGUGCCGUUCCCCAAGA |
| siRNA 895 | 895 | CUUGGGGAACGGCACUGGG | 2946 | CCCAGUGCCGUUCCCCAAG |
| siRNA 896 | 896 | UUGGGGAACGGCACUGGGU | 2947 | ACCCAGUGCCGUUCCCCAA |
| siRNA 897 | 897 | UGGGGAACGGCACUGGGUA | 2948 | UACCCAGUGCCGUUCCCCA |
| siRNA 898 | 898 | GGGGAACGGCACUGGGUAC | 2949 | GUACCCAGUGCCGUUCCCC |
| siRNA 899 | 899 | GGGAACGGCACUGGGUACC | 2950 | GGUACCCAGUGCCGUUCCC |
| siRNA 900 | 900 | GGAACGGCACUGGGUACCG | 2951 | CGGUACCCAGUGCCGUUCC |
| siRNA 901 | 901 | GAACGGCACUGGGUACCGU | 2952 | ACGGUACCCAGUGCCGUUC |
| siRNA 902 | 902 | AACGGCACUGGGUACCGUG | 2953 | CACGGUACCCAGUGCCGUU |
| siRNA 903 | 903 | ACGGCACUGGGUACCGUGG | 2954 | CCACGGUACCCAGUGCCGU |
| siRNA 904 | 904 | CGGCACUGGGUACCGUGGC | 2955 | GCCACGGUACCCAGUGCCG |
| siRNA 905 | 905 | GGCACUGGGUACCGUGGCG | 2956 | CGCCACGGUACCCAGUGCC |
| siRNA 906 | 906 | GCACUGGGUACCGUGGCGU | 2957 | ACGCCACGGUACCCAGUGC |
| siRNA 907 | 907 | CACUGGGUACCGUGGCGUG | 2958 | CACGCCACGGUACCCAGUG |
| siRNA 908 | 908 | ACUGGGUACCGUGGCGUGG | 2959 | CCACGCCACGGUACCCAGU |
| siRNA 909 | 909 | CUGGGUACCGUGGCGUGGC | 2960 | GCCACGCCACGGUACCCAG |
| siRNA 910 | 910 | UGGGUACCGUGGCGUGGCC | 2961 | GGCCACGCCACGGUACCCA |
| siRNA 911 | 911 | GGGUACCGUGGCGUGGCCA | 2962 | UGGCCACGCCACGGUACCC |
| siRNA 912 | 912 | GGUACCGUGGCGUGGCCAG | 2963 | CUGGCCACGCCACGGUACC |
| siRNA 913 | 913 | GUACCGUGGCGUGGCCAGC | 2964 | GCUGGCCACGCCACGGUAC |
| siRNA 914 | 914 | UACCGUGGCGUGGCCAGCA | 2965 | UGCUGGCCACGCCACGGUA |
| siRNA 915 | 915 | ACCGUGGCGUGGCCAGCAC | 2966 | GUGCUGGCCACGCCACGGU |
| siRNA 916 | 916 | CCGUGGCGUGGCCAGCACC | 2967 | GGUGCUGGCCACGCCACGG |
| siRNA 917 | 917 | CGUGGCGUGGCCAGCACCU | 2968 | AGGUGCUGGCCACGCCACG |
| siRNA 918 | 918 | GUGGCGUGGCCAGCACCUC | 2969 | GAGGUGCUGGCCACGCCAC |
| siRNA 919 | 919 | UGGCGUGGCCAGCACCUCA | 2970 | UGAGGUGCUGGCCACGCCA |
| siRNA 920 | 920 | GGCGUGGCCAGCACCUCAG | 2971 | CUGAGGUGCUGGCCACGCC |
| siRNA 921 | 921 | GCGUGGCCAGCACCUCAGC | 2972 | GCUGAGGUGCUGGCCACGC |
| siRNA 922 | 922 | CGUGGCCAGCACCUCAGCC | 2973 | GGCUGAGGUGCUGGCCACG |
| siRNA 923 | 923 | GUGGCCAGCACCUCAGCCU | 2974 | AGGCUGAGGUGCUGGCCAC |
| siRNA 924 | 924 | UGGCCAGCACCUCAGCCUC | 2975 | GAGGCUGAGGUGCUGGCCA |
| siRNA 925 | 925 | GGCCAGCACCUCAGCCUCG | 2976 | CGAGGCUGAGGUGCUGGCC |
| siRNA 926 | 926 | GCCAGCACCUCAGCCUCGG | 2977 | CCGAGGCUGAGGUGCUGGC |
| siRNA 927 | 927 | CCAGCACCUCAGCCUCGGG | 2978 | CCCGAGGCUGAGGUGCUGG |
| siRNA 928 | 928 | CAGCACCUCAGCCUCGGGC | 2979 | GCCCGAGGCUGAGGUGCUG |
| siRNA 929 | 929 | AGCACCUCAGCCUCGGGCC | 2980 | GGCCCGAGGCUGAGGUGCU |
| siRNA 930 | 930 | GCACCUCAGCCUCGGGCCU | 2981 | AGGCCCGAGGCUGAGGUGC |
| siRNA 931 | 931 | CACCUCAGCCUCGGGCCUC | 2982 | GAGGCCCGAGGCUGAGGUG |
| siRNA 932 | 932 | ACCUCAGCCUCGGGCCUCA | 2983 | UGAGGCCCGAGGCUGAGGU |
| siRNA 933 | 933 | CCUCAGCCUCGGGCCUCAG | 2984 | CUGAGGCCCGAGGCUGAGG |
| siRNA 934 | 934 | CUCAGCCUCGGGCCUCAGC | 2985 | GCUGAGGCCCGAGGCUGAG |
| siRNA 935 | 935 | UCAGCCUCGGGCCUCAGCU | 2986 | AGCUGAGGCCCGAGGCUGA |
| siRNA 936 | 936 | CAGCCUCGGGCCUCAGCUG | 2987 | CAGCUGAGGCCCGAGGCUG |
| siRNA 937 | 937 | AGCCUCGGGCCUCAGCUGC | 2988 | GCAGCUGAGGCCCGAGGCU |
| siRNA 938 | 938 | GCCUCGGGCCUCAGCUGCC | 2989 | GGCAGCUGAGGCCCGAGGC |
| siRNA 939 | 939 | CCUCGGGCCUCAGCUGCCU | 2990 | AGGCAGCUGAGGCCCGAGG |
| siRNA 940 | 940 | CUCGGGCCUCAGCUGCCUG | 2991 | CAGGCAGCUGAGGCCCGAG |
| siRNA 941 | 941 | UCGGGCCUCAGCUGCCUGG | 2992 | CCAGGCAGCUGAGGCCCGA |
| siRNA 942 | 942 | CGGGCCUCAGCUGCCUGGC | 2993 | GCCAGGCAGCUGAGGCCCG |
| siRNA 943 | 943 | GGGCCUCAGCUGCCUGGCC | 2994 | GGCCAGGCAGCUGAGGCCC |
| siRNA 944 | 944 | GGCCUCAGCUGCCUGGCCU | 2995 | AGGCCAGGCAGCUGAGGCC |
| siRNA 945 | 945 | GCCUCAGCUGCCUGGCCUG | 2996 | CAGGCCAGGCAGCUGAGGC |
| siRNA 946 | 946 | CCUCAGCUGCCUGGCCUGG | 2997 | CCAGGCCAGGCAGCUGAGG |
| siRNA 947 | 947 | CUCAGCUGCCUGGCCUGGA | 2998 | UCCAGGCCAGGCAGCUGAG |
| siRNA 948 | 948 | UCAGCUGCCUGGCCUGGAA | 2999 | UUCCAGGCCAGGCAGCUGA |
| siRNA 949 | 949 | CAGCUGCCUGGCCUGGAAC | 3000 | GUUCCAGGCCAGGCAGCUG |
| siRNA 950 | 950 | AGCUGCCUGGCCUGGAACU | 3001 | AGUUCCAGGCCAGGCAGCU |
| siRNA 951 | 951 | GCUGCCUGGCCUGGAACUC | 3002 | GAGUUCCAGGCCAGGCAGC |
| siRNA 952 | 952 | CUGCCUGGCCUGGAACUCC | 3003 | GGAGUUCCAGGCCAGGCAG |
| siRNA 953 | 953 | UGCCUGGCCUGGAACUCCG | 3004 | CGGAGUUCCAGGCCAGGCA |
| siRNA 954 | 954 | GCCUGGCCUGGAACUCCGA | 3005 | UCGGAGUUCCAGGCCAGGC |
| siRNA 955 | 955 | CCUGGCCUGGAACUCCGAU | 3006 | AUCGGAGUUCCAGGCCAGG |
| siRNA 956 | 956 | CUGGCCUGGAACUCCGAUC | 3007 | GAUCGGAGUUCCAGGCCAG |
| siRNA 957 | 957 | UGGCCUGGAACUCCGAUCU | 3008 | AGAUCGGAGUUCCAGGCCA |
| siRNA 958 | 958 | GGCCUGGAACUCCGAUCUG | 3009 | CAGAUCGGAGUUCCAGGCC |
| siRNA 959 | 959 | GCCUGGAACUCCGAUCUGC | 3010 | GCAGAUCGGAGUUCCAGGC |
| siRNA 960 | 960 | CCUGGAACUCCGAUCUGCU | 3011 | AGCAGAUCGGAGUUCCAGG |
| siRNA 961 | 961 | CUGGAACUCCGAUCUGCUC | 3012 | GAGCAGAUCGGAGUUCCAG |
| siRNA 962 | 962 | UGGAACUCCGAUCUGCUCU | 3013 | AGAGCAGAUCGGAGUUCCA |
| siRNA 963 | 963 | GGAACUCCGAUCUGCUCUA | 3014 | UAGAGCAGAUCGGAGUUCC |
| siRNA 964 | 964 | GAACUCCGAUCUGCUCUAC | 3015 | GUAGAGCAGAUCGGAGUUC |
| siRNA 965 | 965 | AACUCCGAUCUGCUCUACC | 3016 | GGUAGAGCAGAUCGGAGUU |
| siRNA 966 | 966 | ACUCCGAUCUGCUCUACCA | 3017 | UGGUAGAGCAGAUCGGAGU |
| siRNA 967 | 967 | CUCCGAUCUGCUCUACCAG | 3018 | CUGGUAGAGCAGAUCGGAG |
| siRNA 968 | 968 | UCCGAUCUGCUCUACCAGG | 3019 | CCUGGUAGAGCAGAUCGGA |
| siRNA 969 | 969 | CCGAUCUGCUCUACCAGGA | 3020 | UCCUGGUAGAGCAGAUCGG |
| siRNA 970 | 970 | CGAUCUGCUCUACCAGGAG | 3021 | CUCCUGGUAGAGCAGAUCG |
| siRNA 971 | 971 | GAUCUGCUCUACCAGGAGC | 3022 | GCUCCUGGUAGAGCAGAUC |
| siRNA 972 | 972 | AUCUGCUCUACCAGGAGCU | 3023 | AGCUCCUGGUAGAGCAGAU |
| siRNA 973 | 973 | UCUGCUCUACCAGGAGCUG | 3024 | CAGCUCCUGGUAGAGCAGA |
| siRNA 974 | 974 | CUGCUCUACCAGGAGCUGC | 3025 | GCAGCUCCUGGUAGAGCAG |
| siRNA 975 | 975 | UGCUCUACCAGGAGCUGCA | 3026 | UGCAGCUCCUGGUAGAGCA |
| siRNA 976 | 976 | GCUCUACCAGGAGCUGCAC | 3027 | GUGCAGCUCCUGGUAGAGC |
| siRNA 977 | 977 | CUCUACCAGGAGCUGCACG | 3028 | CGUGCAGCUCCUGGUAGAG |
| siRNA 978 | 978 | UCUACCAGGAGCUGCACGU | 3029 | ACGUGCAGCUCCUGGUAGA |
| siRNA 979 | 979 | CUACCAGGAGCUGCACGUG | 3030 | CACGUGCAGCUCCUGGUAG |
| siRNA 980 | 980 | UACCAGGAGCUGCACGUGG | 3031 | CCACGUGCAGCUCCUGGUA |
| siRNA 981 | 981 | ACCAGGAGCUGCACGUGGA | 3032 | UCCACGUGCAGCUCCUGGU |
| siRNA 982 | 982 | CCAGGAGCUGCACGUGGAC | 3033 | GUCCACGUGCAGCUCCUGG |
| siRNA 983 | 983 | CAGGAGCUGCACGUGGACU | 3034 | AGUCCACGUGCAGCUCCUG |
| siRNA 984 | 984 | AGGAGCUGCACGUGGACUC | 3035 | GAGUCCACGUGCAGCUCCU |
| siRNA 985 | 985 | GGAGCUGCACGUGGACUCC | 3036 | GGAGUCCACGUGCAGCUCC |
| siRNA 986 | 986 | GAGCUGCACGUGGACUCCG | 3037 | CGGAGUCCACGUGCAGCUC |
| siRNA 987 | 987 | AGCUGCACGUGGACUCCGU | 3038 | ACGGAGUCCACGUGCAGCU |
| siRNA 988 | 988 | GCUGCACGUGGACUCCGUG | 3039 | CACGGAGUCCACGUGCAGC |
| siRNA 989 | 989 | CUGCACGUGGACUCCGUGG | 3040 | CCACGGAGUCCACGUGCAG |
| siRNA 990 | 990 | UGCACGUGGACUCCGUGGG | 3041 | CCCACGGAGUCCACGUGCA |
| siRNA 991 | 991 | GCACGUGGACUCCGUGGGC | 3042 | GCCCACGGAGUCCACGUGC |
| siRNA 992 | 992 | CACGUGGACUCCGUGGGCG | 3043 | CGCCCACGGAGUCCACGUG |
| siRNA 993 | 993 | ACGUGGACUCCGUGGGCGC | 3044 | GCGCCCACGGAGUCCACGU |
| siRNA 994 | 994 | CGUGGACUCCGUGGGCGCC | 3045 | GGCGCCCACGGAGUCCACG |
| siRNA 995 | 995 | GUGGACUCCGUGGGCGCCG | 3046 | CGGCGCCCACGGAGUCCAC |
| siRNA 996 | 996 | UGGACUCCGUGGGCGCCGC | 3047 | GCGGCGCCCACGGAGUCCA |
| siRNA 997 | 997 | GGACUCCGUGGGCGCCGCG | 3048 | CGCGGCGCCCACGGAGUCC |
| siRNA 998 | 998 | GACUCCGUGGGCGCCGCGG | 3049 | CCGCGGCGCCCACGGAGUC |
| siRNA 999 | 999 | ACUCCGUGGGCGCCGCGGC | 3050 | GCCGCGGCGCCCACGGAGU |
| siRNA 1000 | 1000 | CUCCGUGGGCGCCGCGGCC | 3051 | GGCCGCGGCGCCCACGGAG |
| siRNA 1001 | 1001 | UCCGUGGGCGCCGCGGCCC | 3052 | GGGCCGCGGCGCCCACGGA |
| siRNA 1002 | 1002 | CCGUGGGCGCCGCGGCCCU | 3053 | AGGGCCGCGGCGCCCACGG |
| siRNA 1003 | 1003 | CGUGGGCGCCGCGGCCCUG | 3054 | CAGGGCCGCGGCGCCCACG |
| siRNA 1004 | 1004 | GUGGGCGCCGCGGCCCUGC | 3055 | GCAGGGCCGCGGCGCCCAC |
| siRNA 1005 | 1005 | UGGGCGCCGCGGCCCUGCU | 3056 | AGCAGGGCCGCGGCGCCCA |
| siRNA 1006 | 1006 | GGGCGCCGCGGCCCUGCUG | 3057 | CAGCAGGGCCGCGGCGCCC |
| siRNA 1007 | 1007 | GGCGCCGCGGCCCUGCUGG | 3058 | CCAGCAGGGCCGCGGCGCC |
| siRNA 1008 | 1008 | GCGCCGCGGCCCUGCUGGG | 3059 | CCCAGCAGGGCCGCGGCGC |
| siRNA 1009 | 1009 | CGCCGCGGCCCUGCUGGGC | 3060 | GCCCAGCAGGGCCGCGGCG |
| siRNA 1010 | 1010 | GCCGCGGCCCUGCUGGGCC | 3061 | GGCCCAGCAGGGCCGCGGC |
| siRNA 1011 | 1011 | CCGCGGCCCUGCUGGGCCU | 3062 | AGGCCCAGCAGGGCCGCGG |
| siRNA 1012 | 1012 | CGCGGCCCUGCUGGGCCUG | 3063 | CAGGCCCAGCAGGGCCGCG |
| siRNA 1013 | 1013 | GCGGCCCUGCUGGGCCUGG | 3064 | CCAGGCCCAGCAGGGCCGC |
| siRNA 1014 | 1014 | CGGCCCUGCUGGGCCUGGG | 3065 | CCCAGGCCCAGCAGGGCCG |
| siRNA 1015 | 1015 | GGCCCUGCUGGGCCUGGGC | 3066 | GCCCAGGCCCAGCAGGGCC |
| siRNA 1016 | 1016 | GCCCUGCUGGGCCUGGGCC | 3067 | GGCCCAGGCCCAGCAGGGC |
| siRNA 1017 | 1017 | CCCUGCUGGGCCUGGGCCC | 3068 | GGGCCCAGGCCCAGCAGGG |
| siRNA 1018 | 1018 | CCUGCUGGGCCUGGGCCCC | 3069 | GGGGCCCAGGCCCAGCAGG |
| siRNA 1019 | 1019 | CUGCUGGGCCUGGGCCCCC | 3070 | GGGGGCCCAGGCCCAGCAG |
| siRNA 1020 | 1020 | UGCUGGGCCUGGGCCCCCA | 3071 | UGGGGGCCCAGGCCCAGCA |
| siRNA 1021 | 1021 | GCUGGGCCUGGGCCCCCAU | 3072 | AUGGGGGCCCAGGCCCAGC |
| siRNA 1022 | 1022 | CUGGGCCUGGGCCCCCAUG | 3073 | CAUGGGGGCCCAGGCCCAG |
| siRNA 1023 | 1023 | UGGGCCUGGGCCCCCAUGC | 3074 | GCAUGGGGGCCCAGGCCCA |
| siRNA 1024 | 1024 | GGGCCUGGGCCCCCAUGCC | 3075 | GGCAUGGGGGCCCAGGCCC |
| siRNA 1025 | 1025 | GGCCUGGGCCCCCAUGCCU | 3076 | AGGCAUGGGGGCCCAGGCC |
| siRNA 1026 | 1026 | GCCUGGGCCCCCAUGCCUA | 3077 | UAGGCAUGGGGGCCCAGGC |
| siRNA 1027 | 1027 | CCUGGGCCCCCAUGCCUAC | 3078 | GUAGGCAUGGGGGCCCAGG |
| siRNA 1028 | 1028 | CUGGGCCCCCAUGCCUACU | 3079 | AGUAGGCAUGGGGGCCCAG |
| siRNA 1029 | 1029 | UGGGCCCCCAUGCCUACUG | 3080 | CAGUAGGCAUGGGGGCCCA |
| siRNA 1030 | 1030 | GGGCCCCCAUGCCUACUGC | 3081 | GCAGUAGGCAUGGGGGCCC |
| siRNA 1031 | 1031 | GGCCCCCAUGCCUACUGCC | 3082 | GGCAGUAGGCAUGGGGGCC |
| siRNA 1032 | 1032 | GCCCCCAUGCCUACUGCCG | 3083 | CGGCAGUAGGCAUGGGGGC |
| siRNA 1033 | 1033 | CCCCCAUGCCUACUGCCGG | 3084 | CCGGCAGUAGGCAUGGGGG |
| siRNA 1034 | 1034 | CCCCAUGCCUACUGCCGGA | 3085 | UCCGGCAGUAGGCAUGGGG |
| siRNA 1035 | 1035 | CCCAUGCCUACUGCCGGAA | 3086 | UUCCGGCAGUAGGCAUGGG |
| siRNA 1036 | 1036 | CCAUGCCUACUGCCGGAAU | 3087 | AUUCCGGCAGUAGGCAUGG |
| siRNA 1037 | 1037 | CAUGCCUACUGCCGGAAUC | 3088 | GAUUCCGGCAGUAGGCAUG |
| siRNA 1038 | 1038 | AUGCCUACUGCCGGAAUCC | 3089 | GGAUUCCGGCAGUAGGCAU |
| SiRNA 1039 | 1039 | UGCCUACUGCCGGAAUCCG | 3090 | CGGAUUCCGGCAGUAGGCA |
| siRNA 1040 | 1040 | GCCUACUGCCGGAAUCCGG | 3091 | CCGGAUUCCGGCAGUAGGC |
| siRNA 1041 | 1041 | CCUACUGCCGGAAUCCGGA | 3092 | UCCGGAUUCCGGCAGUAGG |
| siRNA 1042 | 1042 | CUACUGCCGGAAUCCGGAC | 3093 | GUCCGGAUUCCGGCAGUAG |
| siRNA 1043 | 1043 | UACUGCCGGAAUCCGGACA | 3094 | UGUCCGGAUUCCGGCAGUA |
| siRNA 1044 | 1044 | ACUGCCGGAAUCCGGACAA | 3095 | UUGUCCGGAUUCCGGCAGU |
| siRNA 1045 | 1045 | CUGCCGGAAUCCGGACAAU | 3096 | AUUGUCCGGAUUCCGGCAG |
| siRNA 1046 | 1046 | UGCCGGAAUCCGGACAAUG | 3097 | CAUUGUCCGGAUUCCGGCA |
| siRNA 1047 | 1047 | GCCGGAAUCCGGACAAUGA | 3098 | UCAUUGUCCGGAUUCCGGC |
| siRNA 1048 | 1048 | CCGGAAUCCGGACAAUGAC | 3099 | GUCAUUGUCCGGAUUCCGG |
| siRNA 1049 | 1049 | CGGAAUCCGGACAAUGACG | 3100 | CGUCAUUGUCCGGAUUCCG |
| siRNA 1050 | 1050 | GGAAUCCGGACAAUGACGA | 3101 | UCGUCAUUGUCCGGAUUCC |
| siRNA 1051 | 1051 | GAAUCCGGACAAUGACGAG | 3102 | CUCGUCAUUGUCCGGAUUC |
| siRNA 1052 | 1052 | AAUCCGGACAAUGACGAGA | 3103 | UCUCGUCAUUGUCCGGAUU |
| siRNA 1053 | 1053 | AUCCGGACAAUGACGAGAG | 3104 | CUCUCGUCAUUGUCCGGAU |
| siRNA 1054 | 1054 | UCCGGACAAUGACGAGAGG | 3105 | CCUCUCGUCAUUGUCCGGA |
| siRNA 1055 | 1055 | CCGGACAAUGACGAGAGGC | 3106 | GCCUCUCGUCAUUGUCCGG |
| siRNA 1056 | 1056 | CGGACAAUGACGAGAGGCC | 3107 | GGCCUCUCGUCAUUGUCCG |
| siRNA 1057 | 1057 | GGACAAUGACGAGAGGCCC | 3108 | GGGCCUCUCGUCAUUGUCC |
| siRNA 1058 | 1058 | GACAAUGACGAGAGGCCCU | 3109 | AGGGCCUCUCGUCAUUGUC |
| siRNA 1059 | 1059 | ACAAUGACGAGAGGCCCUG | 3110 | CAGGGCCUCUCGUCAUUGU |
| siRNA 1060 | 1060 | CAAUGACGAGAGGCCCUGG | 3111 | CCAGGGCCUCUCGUCAUUG |
| siRNA 1061 | 1061 | AAUGACGAGAGGCCCUGGU | 3112 | ACCAGGGCCUCUCGUCAUU |
| siRNA 1062 | 1062 | AUGACGAGAGGCCCUGGUG | 3113 | CACCAGGGCCUCUCGUCAU |
| siRNA 1063 | 1063 | UGACGAGAGGCCCUGGUGC | 3114 | GCACCAGGGCCUCUCGUCA |
| siRNA 1064 | 1064 | GACGAGAGGCCCUGGUGCU | 3115 | AGCACCAGGGCCUCUCGUC |
| siRNA 1065 | 1065 | ACGAGAGGCCCUGGUGCUA | 3116 | UAGCACCAGGGCCUCUCGU |
| siRNA 1066 | 1066 | CGAGAGGCCCUGGUGCUAC | 3117 | GUAGCACCAGGGCCUCUCG |
| siRNA 1067 | 1067 | GAGAGGCCCUGGUGCUACG | 3118 | CGUAGCACCAGGGCCUCUC |
| siRNA 1068 | 1068 | AGAGGCCCUGGUGCUACGU | 3119 | ACGUAGCACCAGGGCCUCU |
| siRNA 1069 | 1069 | GAGGCCCUGGUGCUACGUG | 3120 | CACGUAGCACCAGGGCCUC |
| siRNA 1070 | 1070 | AGGCCCUGGUGCUACGUGG | 3121 | CCACGUAGCACCAGGGCCU |
| siRNA 1071 | 1071 | GGCCCUGGUGCUACGUGGU | 3122 | ACCACGUAGCACCAGGGCC |
| SiRNA 1072 | 1072 | GCCCUGGUGCUACGUGGUG | 3123 | CACCACGUAGCACCAGGGC |
| siRNA 1073 | 1073 | CCCUGGUGCUACGUGGUGA | 3124 | UCACCACGUAGCACCAGGG |
| siRNA 1074 | 1074 | CCUGGUGCUACGUGGUGAA | 3125 | UUCACCACGUAGCACCAGG |
| siRNA 1075 | 1075 | CUGGUGCUACGUGGUGAAG | 3126 | CUUCACCACGUAGCACCAG |
| siRNA 1076 | 1076 | UGGUGCUACGUGGUGAAGG | 3127 | CCUUCACCACGUAGCACCA |
| siRNA 1077 | 1077 | GGUGCUACGUGGUGAAGGA | 3128 | UCCUUCACCACGUAGCACC |
| siRNA 1078 | 1078 | GUGCUACGUGGUGAAGGAC | 3129 | GUCCUUCACCACGUAGCAC |
| SiRNA 1079 | 1079 | UGCUACGUGGUGAAGGACA | 3130 | UGUCCUUCACCACGUAGCA |
| siRNA 1080 | 1080 | GCUACGUGGUGAAGGACAG | 3131 | CUGUCCUUCACCACGUAGC |
| siRNA 1081 | 1081 | CUACGUGGUGAAGGACAGC | 3132 | GCUGUCCUUCACCACGUAG |
| siRNA 1082 | 1082 | UACGUGGUGAAGGACAGCG | 3133 | CGCUGUCCUUCACCACGUA |
| siRNA 1083 | 1083 | ACGUGGUGAAGGACAGCGC | 3134 | GCGCUGUCCUUCACCACGU |
| siRNA 1084 | 1084 | CGUGGUGAAGGACAGCGCG | 3135 | CGCGCUGUCCUUCACCACG |
| siRNA 1085 | 1085 | GUGGUGAAGGACAGCGCGC | 3136 | GCGCGCUGUCCUUCACCAC |
| siRNA 1086 | 1086 | UGGUGAAGGACAGCGCGCU | 3137 | AGCGCGCUGUCCUUCACCA |
| siRNA 1087 | 1087 | GGUGAAGGACAGCGCGCUC | 3138 | GAGCGCGCUGUCCUUCACC |
| siRNA 1088 | 1088 | GUGAAGGACAGCGCGCUCU | 3139 | AGAGCGCGCUGUCCUUCAC |
| siRNA 1089 | 1089 | UGAAGGACAGCGCGCUCUC | 3140 | GAGAGCGCGCUGUCCUUCA |
| siRNA 1090 | 1090 | GAAGGACAGCGCGCUCUCC | 3141 | GGAGAGCGCGCUGUCCUUC |
| siRNA 1091 | 1091 | AAGGACAGCGCGCUCUCCU | 3142 | AGGAGAGCGCGCUGUCCUU |
| siRNA 1092 | 1092 | AGGACAGCGCGCUCUCCUG | 3143 | CAGGAGAGCGCGCUGUCCU |
| siRNA 1093 | 1093 | GGACAGCGCGCUCUCCUGG | 3144 | CCAGGAGAGCGCGCUGUCC |
| siRNA 1094 | 1094 | GACAGCGCGCUCUCCUGGG | 3145 | CCCAGGAGAGCGCGCUGUC |
| siRNA 1095 | 1095 | ACAGCGCGCUCUCCUGGGA | 3146 | UCCCAGGAGAGCGCGCUGU |
| siRNA 1096 | 1096 | CAGCGCGCUCUCCUGGGAG | 3147 | CUCCCAGGAGAGCGCGCUG |
| siRNA 1097 | 1097 | AGCGCGCUCUCCUGGGAGU | 3148 | ACUCCCAGGAGAGCGCGCU |
| siRNA 1098 | 1098 | GCGCGCUCUCCUGGGAGUA | 3149 | UACUCCCAGGAGAGCGCGC |
| siRNA 1099 | 1099 | CGCGCUCUCCUGGGAGUAC | 3150 | GUACUCCCAGGAGAGCGCG |
| siRNA 1100 | 1100 | GCGCUCUCCUGGGAGUACU | 3151 | AGUACUCCCAGGAGAGCGC |
| siRNA 1101 | 1101 | CGCUCUCCUGGGAGUACUG | 3152 | CAGUACUCCCAGGAGAGCG |
| siRNA 1102 | 1102 | GCUCUCCUGGGAGUACUGC | 3153 | GCAGUACUCCCAGGAGAGC |
| siRNA 1103 | 1103 | CUCUCCUGGGAGUACUGCC | 3154 | GGCAGUACUCCCAGGAGAG |
| siRNA 1104 | 1104 | UCUCCUGGGAGUACUGCCG | 3155 | CGGCAGUACUCCCAGGAGA |
| siRNA 1105 | 1105 | CUCCUGGGAGUACUGCCGC | 3156 | GCGGCAGUACUCCCAGGAG |
| siRNA 1106 | 1106 | UCCUGGGAGUACUGCCGCC | 3157 | GGCGGCAGUACUCCCAGGA |
| siRNA 1107 | 1107 | CCUGGGAGUACUGCCGCCU | 3158 | AGGCGGCAGUACUCCCAGG |
| siRNA 1108 | 1108 | CUGGGAGUACUGCCGCCUG | 3159 | CAGGCGGCAGUACUCCCAG |
| siRNA 1109 | 1109 | UGGGAGUACUGCCGCCUGG | 3160 | CCAGGCGGCAGUACUCCCA |
| siRNA 1110 | 1110 | GGGAGUACUGCCGCCUGGA | 3161 | UCCAGGCGGCAGUACUCCC |
| siRNA 1111 | 1111 | GGAGUACUGCCGCCUGGAG | 3162 | CUCCAGGCGGCAGUACUCC |
| siRNA 1112 | 1112 | GAGUACUGCCGCCUGGAGG | 3163 | CCUCCAGGCGGCAGUACUC |
| siRNA 1113 | 1113 | AGUACUGCCGCCUGGAGGC | 3164 | GCCUCCAGGCGGCAGUACU |
| SiRNA 1114 | 1114 | GUACUGCCGCCUGGAGGCC | 3165 | GGCCUCCAGGCGGCAGUAC |
| siRNA 1115 | 1115 | UACUGCCGCCUGGAGGCCU | 3166 | AGGCCUCCAGGCGGCAGUA |
| siRNA 1116 | 1116 | ACUGCCGCCUGGAGGCCUG | 3167 | CAGGCCUCCAGGCGGCAGU |
| siRNA 1117 | 1117 | CUGCCGCCUGGAGGCCUGC | 3168 | GCAGGCCUCCAGGCGGCAG |
| siRNA 1118 | 1118 | UGCCGCCUGGAGGCCUGCG | 3169 | CGCAGGCCUCCAGGCGGCA |
| siRNA 1119 | 1119 | GCCGCCUGGAGGCCUGCGA | 3170 | UCGCAGGCCUCCAGGCGGC |
| siRNA 1120 | 1120 | CCGCCUGGAGGCCUGCGAA | 3171 | UUCGCAGGCCUCCAGGCGG |
| siRNA 1121 | 1121 | CGCCUGGAGGCCUGCGAAU | 3172 | AUUCGCAGGCCUCCAGGCG |
| siRNA 1122 | 1122 | GCCUGGAGGCCUGCGAAUC | 3173 | GAUUCGCAGGCCUCCAGGC |
| siRNA 1123 | 1123 | CCUGGAGGCCUGCGAAUCC | 3174 | GGAUUCGCAGGCCUCCAGG |
| siRNA 1124 | 1124 | CUGGAGGCCUGCGAAUCCC | 3175 | GGGAUUCGCAGGCCUCCAG |
| siRNA 1125 | 1125 | UGGAGGCCUGCGAAUCCCU | 3176 | AGGGAUUCGCAGGCCUCCA |
| siRNA 1126 | 1126 | GGAGGCCUGCGAAUCCCUC | 3177 | GAGGGAUUCGCAGGCCUCC |
| siRNA 1127 | 1127 | GAGGCCUGCGAAUCCCUCA | 3178 | UGAGGGAUUCGCAGGCCUC |
| siRNA 1128 | 1128 | AGGCCUGCGAAUCCCUCAC | 3179 | GUGAGGGAUUCGCAGGCCU |
| siRNA 1129 | 1129 | GGCCUGCGAAUCCCUCACC | 3180 | GGUGAGGGAUUCGCAGGCC |
| siRNA 1130 | 1130 | GCCUGCGAAUCCCUCACCA | 3181 | UGGUGAGGGAUUCGCAGGC |
| siRNA 1131 | 1131 | CCUGCGAAUCCCUCACCAG | 3182 | CUGGUGAGGGAUUCGCAGG |
| siRNA 1132 | 1132 | CUGCGAAUCCCUCACCAGA | 3183 | UCUGGUGAGGGAUUCGCAG |
| siRNA 1133 | 1133 | UGCGAAUCCCUCACCAGAG | 3184 | CUCUGGUGAGGGAUUCGCA |
| siRNA 1134 | 1134 | GCGAAUCCCUCACCAGAGU | 3185 | ACUCUGGUGAGGGAUUCGC |
| siRNA 1135 | 1135 | CGAAUCCCUCACCAGAGUC | 3186 | GACUCUGGUGAGGGAUUCG |
| siRNA 1136 | 1136 | GAAUCCCUCACCAGAGUCC | 3187 | GGACUCUGGUGAGGGAUUC |
| siRNA 1137 | 1137 | AAUCCCUCACCAGAGUCCA | 3188 | UGGACUCUGGUGAGGGAUU |
| siRNA 1138 | 1138 | AUCCCUCACCAGAGUCCAA | 3189 | UUGGACUCUGGUGAGGGAU |
| siRNA 1139 | 1139 | UCCCUCACCAGAGUCCAAC | 3190 | GUUGGACUCUGGUGAGGGA |
| siRNA 1140 | 1140 | CCCUCACCAGAGUCCAACU | 3191 | AGUUGGACUCUGGUGAGGG |
| siRNA 1141 | 1141 | CCUCACCAGAGUCCAACUG | 3192 | CAGUUGGACUCUGGUGAGG |
| siRNA 1142 | 1142 | CUCACCAGAGUCCAACUGU | 3193 | ACAGUUGGACUCUGGUGAG |
| SiRNA 1143 | 1143 | UCACCAGAGUCCAACUGUC | 3194 | GACAGUUGGACUCUGGUGA |
| siRNA 1144 | 1144 | CACCAGAGUCCAACUGUCA | 3195 | UGACAGUUGGACUCUGGUG |
| siRNA 1145 | 1145 | ACCAGAGUCCAACUGUCAC | 3196 | GUGACAGUUGGACUCUGGU |
| siRNA 1146 | 1146 | CCAGAGUCCAACUGUCACC | 3197 | GGUGACAGUUGGACUCUGG |
| siRNA 1147 | 1147 | CAGAGUCCAACUGUCACCG | 3198 | CGGUGACAGUUGGACUCUG |
| siRNA 1148 | 1148 | AGAGUCCAACUGUCACCGG | 3199 | CCGGUGACAGUUGGACUCU |
| SiRNA 1149 | 1149 | GAGUCCAACUGUCACCGGA | 3200 | UCCGGUGACAGUUGGACUC |
| siRNA 1150 | 1150 | AGUCCAACUGUCACCGGAU | 3201 | AUCCGGUGACAGUUGGACU |
| siRNA 1151 | 1151 | GUCCAACUGUCACCGGAUC | 3202 | GAUCCGGUGACAGUUGGAC |
| siRNA 1152 | 1152 | UCCAACUGUCACCGGAUCU | 3203 | AGAUCCGGUGACAGUUGGA |
| siRNA 1153 | 1153 | CCAACUGUCACCGGAUCUC | 3204 | GAGAUCCGGUGACAGUUGG |
| siRNA 1154 | 1154 | CAACUGUCACCGGAUCUCC | 3205 | GGAGAUCCGGUGACAGUUG |
| siRNA 1155 | 1155 | AACUGUCACCGGAUCUCCU | 3206 | AGGAGAUCCGGUGACAGUU |
| siRNA 1156 | 1156 | ACUGUCACCGGAUCUCCUG | 3207 | CAGGAGAUCCGGUGACAGU |
| siRNA 1157 | 1157 | CUGUCACCGGAUCUCCUGG | 3208 | CCAGGAGAUCCGGUGACAG |
| siRNA 1158 | 1158 | UGUCACCGGAUCUCCUGGC | 3209 | GCCAGGAGAUCCGGUGACA |
| siRNA 1159 | 1159 | GUCACCGGAUCUCCUGGCG | 3210 | CGCCAGGAGAUCCGGUGAC |
| siRNA 1160 | 1160 | UCACCGGAUCUCCUGGCGA | 3211 | UCGCCAGGAGAUCCGGUGA |
| siRNA 1161 | 1161 | CACCGGAUCUCCUGGCGAC | 3212 | GUCGCCAGGAGAUCCGGUG |
| siRNA 1162 | 1162 | ACCGGAUCUCCUGGCGACC | 3213 | GGUCGCCAGGAGAUCCGGU |
| siRNA 1163 | 1163 | CCGGAUCUCCUGGCGACCC | 3214 | GGGUCGCCAGGAGAUCCGG |
| siRNA 1164 | 1164 | CGGAUCUCCUGGCGACCCU | 3215 | AGGGUCGCCAGGAGAUCCG |
| siRNA 1165 | 1165 | GGAUCUCCUGGCGACCCUG | 3216 | CAGGGUCGCCAGGAGAUCC |
| siRNA 1166 | 1166 | GAUCUCCUGGCGACCCUGC | 3217 | GCAGGGUCGCCAGGAGAUC |
| siRNA 1167 | 1167 | AUCUCCUGGCGACCCUGCC | 3218 | GGCAGGGUCGCCAGGAGAU |
| siRNA 1168 | 1168 | UCUCCUGGCGACCCUGCCU | 3219 | AGGCAGGGUCGCCAGGAGA |
| siRNA 1169 | 1169 | CUCCUGGCGACCCUGCCUG | 3220 | CAGGCAGGGUCGCCAGGAG |
| siRNA 1170 | 1170 | UCCUGGCGACCCUGCCUGA | 3221 | UCAGGCAGGGUCGCCAGGA |
| siRNA 1171 | 1171 | CCUGGCGACCCUGCCUGAG | 3222 | CUCAGGCAGGGUCGCCAGG |
| siRNA 1172 | 1172 | CUGGCGACCCUGCCUGAGC | 3223 | GCUCAGGCAGGGUCGCCAG |
| siRNA 1173 | 1173 | UGGCGACCCUGCCUGAGCC | 3224 | GGCUCAGGCAGGGUCGCCA |
| siRNA 1174 | 1174 | GGCGACCCUGCCUGAGCCA | 3225 | UGGCUCAGGCAGGGUCGCC |
| siRNA 1175 | 1175 | GCGACCCUGCCUGAGCCAG | 3226 | CUGGCUCAGGCAGGGUCGC |
| siRNA 1176 | 1176 | CGACCCUGCCUGAGCCAGC | 3227 | GCUGGCUCAGGCAGGGUCG |
| siRNA 1177 | 1177 | GACCCUGCCUGAGCCAGCC | 3228 | GGCUGGCUCAGGCAGGGUC |
| siRNA 1178 | 1178 | ACCCUGCCUGAGCCAGCCU | 3229 | AGGCUGGCUCAGGCAGGGU |
| siRNA 1179 | 1179 | CCCUGCCUGAGCCAGCCUC | 3230 | GAGGCUGGCUCAGGCAGGG |
| siRNA 1180 | 1180 | CCUGCCUGAGCCAGCCUCC | 3231 | GGAGGCUGGCUCAGGCAGG |
| siRNA 1181 | 1181 | CUGCCUGAGCCAGCCUCCC | 3232 | GGGAGGCUGGCUCAGGCAG |
| siRNA 1182 | 1182 | UGCCUGAGCCAGCCUCCCC | 3233 | GGGGAGGCUGGCUCAGGCA |
| siRNA 1183 | 1183 | GCCUGAGCCAGCCUCCCCG | 3234 | CGGGGAGGCUGGCUCAGGC |
| siRNA 1184 | 1184 | CCUGAGCCAGCCUCCCCGG | 3235 | CCGGGGAGGCUGGCUCAGG |
| siRNA 1185 | 1185 | CUGAGCCAGCCUCCCCGGG | 3236 | CCCGGGGAGGCUGGCUCAG |
| siRNA 1186 | 1186 | UGAGCCAGCCUCCCCGGGG | 3237 | CCCCGGGGAGGCUGGCUCA |
| siRNA 1187 | 1187 | GAGCCAGCCUCCCCGGGGC | 3238 | GCCCCGGGGAGGCUGGCUC |
| siRNA 1188 | 1188 | AGCCAGCCUCCCCGGGGCG | 3239 | CGCCCCGGGGAGGCUGGCU |
| siRNA 1189 | 1189 | GCCAGCCUCCCCGGGGCGC | 3240 | GCGCCCCGGGGAGGCUGGC |
| siRNA 1190 | 1190 | CCAGCCUCCCCGGGGCGCC | 3241 | GGCGCCCCGGGGAGGCUGG |
| siRNA 1191 | 1191 | CAGCCUCCCCGGGGCGCCA | 3242 | UGGCGCCCCGGGGAGGCUG |
| siRNA 1192 | 1192 | AGCCUCCCCGGGGCGCCAG | 3243 | CUGGCGCCCCGGGGAGGCU |
| siRNA 1193 | 1193 | GCCUCCCCGGGGCGCCAGG | 3244 | CCUGGCGCCCCGGGGAGGC |
| siRNA 1194 | 1194 | CCUCCCCGGGGCGCCAGGC | 3245 | GCCUGGCGCCCCGGGGAGG |
| siRNA 1195 | 1195 | CUCCCCGGGGCGCCAGGCC | 3246 | GGCCUGGCGCCCCGGGGAG |
| siRNA 1196 | 1196 | UCCCCGGGGCGCCAGGCCU | 3247 | AGGCCUGGCGCCCCGGGGA |
| siRNA 1197 | 1197 | CCCCGGGGCGCCAGGCCUG | 3248 | CAGGCCUGGCGCCCCGGGG |
| siRNA 1198 | 1198 | CCCGGGGCGCCAGGCCUGC | 3249 | GCAGGCCUGGCGCCCCGGG |
| siRNA 1199 | 1199 | CCGGGGCGCCAGGCCUGCG | 3250 | CGCAGGCCUGGCGCCCCGG |
| siRNA 1200 | 1200 | CGGGGCGCCAGGCCUGCGG | 3251 | CCGCAGGCCUGGCGCCCCG |
| siRNA 1201 | 1201 | GGGGCGCCAGGCCUGCGGC | 3252 | GCCGCAGGCCUGGCGCCCC |
| siRNA 1202 | 1202 | GGGCGCCAGGCCUGCGGCA | 3253 | UGCCGCAGGCCUGGCGCCC |
| siRNA 1203 | 1203 | GGCGCCAGGCCUGCGGCAG | 3254 | CUGCCGCAGGCCUGGCGCC |
| siRNA 1204 | 1204 | GCGCCAGGCCUGCGGCAGG | 3255 | CCUGCCGCAGGCCUGGCGC |
| siRNA 1205 | 1205 | CGCCAGGCCUGCGGCAGGA | 3256 | UCCUGCCGCAGGCCUGGCG |
| siRNA 1206 | 1206 | GCCAGGCCUGCGGCAGGAG | 3257 | CUCCUGCCGCAGGCCUGGC |
| siRNA 1207 | 1207 | CCAGGCCUGCGGCAGGAGG | 3258 | CCUCCUGCCGCAGGCCUGG |
| siRNA 1208 | 1208 | CAGGCCUGCGGCAGGAGGC | 3259 | GCCUCCUGCCGCAGGCCUG |
| siRNA 1209 | 1209 | AGGCCUGCGGCAGGAGGCA | 3260 | UGCCUCCUGCCGCAGGCCU |
| siRNA 1210 | 1210 | GGCCUGCGGCAGGAGGCAC | 3261 | GUGCCUCCUGCCGCAGGCC |
| siRNA 1211 | 1211 | GCCUGCGGCAGGAGGCACA | 3262 | UGUGCCUCCUGCCGCAGGC |
| siRNA 1212 | 1212 | CCUGCGGCAGGAGGCACAA | 3263 | UUGUGCCUCCUGCCGCAGG |
| siRNA 1213 | 1213 | CUGCGGCAGGAGGCACAAG | 3264 | CUUGUGCCUCCUGCCGCAG |
| siRNA 1214 | 1214 | UGCGGCAGGAGGCACAAGA | 3265 | UCUUGUGCCUCCUGCCGCA |
| siRNA 1215 | 1215 | GCGGCAGGAGGCACAAGAA | 3266 | UUCUUGUGCCUCCUGCCGC |
| siRNA 1216 | 1216 | CGGCAGGAGGCACAAGAAG | 3267 | CUUCUUGUGCCUCCUGCCG |
| siRNA 1217 | 1217 | GGCAGGAGGCACAAGAAGA | 3268 | UCUUCUUGUGCCUCCUGCC |
| siRNA 1218 | 1218 | GCAGGAGGCACAAGAAGAG | 3269 | CUCUUCUUGUGCCUCCUGC |
| siRNA 1219 | 1219 | CAGGAGGCACAAGAAGAGG | 3270 | CCUCUUCUUGUGCCUCCUG |
| siRNA 1220 | 1220 | AGGAGGCACAAGAAGAGGA | 3271 | UCCUCUUCUUGUGCCUCCU |
| siRNA 1221 | 1221 | GGAGGCACAAGAAGAGGAC | 3272 | GUCCUCUUCUUGUGCCUCC |
| siRNA 1222 | 1222 | GAGGCACAAGAAGAGGACG | 3273 | CGUCCUCUUCUUGUGCCUC |
| siRNA 1223 | 1223 | AGGCACAAGAAGAGGACGU | 3274 | ACGUCCUCUUCUUGUGCCU |
| siRNA 1224 | 1224 | GGCACAAGAAGAGGACGUU | 3275 | AACGUCCUCUUCUUGUGCC |
| siRNA 1225 | 1225 | GCACAAGAAGAGGACGUUC | 3276 | GAACGUCCUCUUCUUGUGC |
| siRNA 1226 | 1226 | CACAAGAAGAGGACGUUCC | 3277 | GGAACGUCCUCUUCUUGUG |
| siRNA 1227 | 1227 | ACAAGAAGAGGACGUUCCU | 3278 | AGGAACGUCCUCUUCUUGU |
| siRNA 1228 | 1228 | CAAGAAGAGGACGUUCCUG | 3279 | CAGGAACGUCCUCUUCUUG |
| siRNA 1229 | 1229 | AAGAAGAGGACGUUCCUGC | 3280 | GCAGGAACGUCCUCUUCUU |
| siRNA 1230 | 1230 | AGAAGAGGACGUUCCUGCG | 3281 | CGCAGGAACGUCCUCUUCU |
| siRNA 1231 | 1231 | GAAGAGGACGUUCCUGCGG | 3282 | CCGCAGGAACGUCCUCUUC |
| siRNA 1232 | 1232 | AAGAGGACGUUCCUGCGGC | 3283 | GCCGCAGGAACGUCCUCUU |
| siRNA 1233 | 1233 | AGAGGACGUUCCUGCGGCC | 3284 | GGCCGCAGGAACGUCCUCU |
| siRNA 1234 | 1234 | GAGGACGUUCCUGCGGCCA | 3285 | UGGCCGCAGGAACGUCCUC |
| siRNA 1235 | 1235 | AGGACGUUCCUGCGGCCAC | 3286 | GUGGCCGCAGGAACGUCCU |
| siRNA 1236 | 1236 | GGACGUUCCUGCGGCCACG | 3287 | CGUGGCCGCAGGAACGUCC |
| siRNA 1237 | 1237 | GACGUUCCUGCGGCCACGU | 3288 | ACGUGGCCGCAGGAACGUC |
| siRNA 1238 | 1238 | ACGUUCCUGCGGCCACGUA | 3289 | UACGUGGCCGCAGGAACGU |
| siRNA 1239 | 1239 | CGUUCCUGCGGCCACGUAU | 3290 | AUACGUGGCCGCAGGAACG |
| siRNA 1240 | 1240 | GUUCCUGCGGCCACGUAUC | 3291 | GAUACGUGGCCGCAGGAAC |
| SiRNA 1241 | 1241 | UUCCUGCGGCCACGUAUCA | 3292 | UGAUACGUGGCCGCAGGAA |
| siRNA 1242 | 1242 | UCCUGCGGCCACGUAUCAU | 3293 | AUGAUACGUGGCCGCAGGA |
| siRNA 1243 | 1243 | CCUGCGGCCACGUAUCAUC | 3294 | GAUGAUACGUGGCCGCAGG |
| siRNA 1244 | 1244 | CUGCGGCCACGUAUCAUCG | 3295 | CGAUGAUACGUGGCCGCAG |
| siRNA 1245 | 1245 | UGCGGCCACGUAUCAUCGG | 3296 | CCGAUGAUACGUGGCCGCA |
| siRNA 1246 | 1246 | GCGGCCACGUAUCAUCGGC | 3297 | GCCGAUGAUACGUGGCCGC |
| siRNA 1247 | 1247 | CGGCCACGUAUCAUCGGCG | 3298 | CGCCGAUGAUACGUGGCCG |
| siRNA 1248 | 1248 | GGCCACGUAUCAUCGGCGG | 3299 | CCGCCGAUGAUACGUGGCC |
| siRNA 1249 | 1249 | GCCACGUAUCAUCGGCGGC | 3300 | GCCGCCGAUGAUACGUGGC |
| siRNA 1250 | 1250 | CCACGUAUCAUCGGCGGCU | 3301 | AGCCGCCGAUGAUACGUGG |
| siRNA 1251 | 1251 | CACGUAUCAUCGGCGGCUC | 3302 | GAGCCGCCGAUGAUACGUG |
| siRNA 1252 | 1252 | ACGUAUCAUCGGCGGCUCC | 3303 | GGAGCCGCCGAUGAUACGU |
| siRNA 1253 | 1253 | CGUAUCAUCGGCGGCUCCU | 3304 | AGGAGCCGCCGAUGAUACG |
| siRNA 1254 | 1254 | GUAUCAUCGGCGGCUCCUC | 3305 | GAGGAGCCGCCGAUGAUAC |
| siRNA 1255 | 1255 | UAUCAUCGGCGGCUCCUCC | 3306 | GGAGGAGCCGCCGAUGAUA |
| siRNA 1256 | 1256 | AUCAUCGGCGGCUCCUCCU | 3307 | AGGAGGAGCCGCCGAUGAU |
| siRNA 1257 | 1257 | UCAUCGGCGGCUCCUCCUC | 3308 | GAGGAGGAGCCGCCGAUGA |
| siRNA 1258 | 1258 | CAUCGGCGGCUCCUCCUCG | 3309 | CGAGGAGGAGCCGCCGAUG |
| siRNA 1259 | 1259 | AUCGGCGGCUCCUCCUCGC | 3310 | GCGAGGAGGAGCCGCCGAU |
| siRNA 1260 | 1260 | UCGGCGGCUCCUCCUCGCU | 3311 | AGCGAGGAGGAGCCGCCGA |
| siRNA 1261 | 1261 | CGGCGGCUCCUCCUCGCUG | 3312 | CAGCGAGGAGGAGCCGCCG |
| siRNA 1262 | 1262 | GGCGGCUCCUCCUCGCUGC | 3313 | GCAGCGAGGAGGAGCCGCC |
| siRNA 1263 | 1263 | GCGGCUCCUCCUCGCUGCC | 3314 | GGCAGCGAGGAGGAGCCGC |
| siRNA 1264 | 1264 | CGGCUCCUCCUCGCUGCCC | 3315 | GGGCAGCGAGGAGGAGCCG |
| siRNA 1265 | 1265 | GGCUCCUCCUCGCUGCCCG | 3316 | CGGGCAGCGAGGAGGAGCC |
| siRNA 1266 | 1266 | GCUCCUCCUCGCUGCCCGG | 3317 | CCGGGCAGCGAGGAGGAGC |
| siRNA 1267 | 1267 | CUCCUCCUCGCUGCCCGGC | 3318 | GCCGGGCAGCGAGGAGGAG |
| siRNA 1268 | 1268 | UCCUCCUCGCUGCCCGGCU | 3319 | AGCCGGGCAGCGAGGAGGA |
| siRNA 1269 | 1269 | CCUCCUCGCUGCCCGGCUC | 3320 | GAGCCGGGCAGCGAGGAGG |
| siRNA 1270 | 1270 | CUCCUCGCUGCCCGGCUCG | 3321 | CGAGCCGGGCAGCGAGGAG |
| siRNA 1271 | 1271 | UCCUCGCUGCCCGGCUCGC | 3322 | GCGAGCCGGGCAGCGAGGA |
| siRNA 1272 | 1272 | CCUCGCUGCCCGGCUCGCA | 3323 | UGCGAGCCGGGCAGCGAGG |
| siRNA 1273 | 1273 | CUCGCUGCCCGGCUCGCAC | 3324 | GUGCGAGCCGGGCAGCGAG |
| siRNA 1274 | 1274 | UCGCUGCCCGGCUCGCACC | 3325 | GGUGCGAGCCGGGCAGCGA |
| siRNA 1275 | 1275 | CGCUGCCCGGCUCGCACCC | 3326 | GGGUGCGAGCCGGGCAGCG |
| siRNA 1276 | 1276 | GCUGCCCGGCUCGCACCCC | 3327 | GGGGUGCGAGCCGGGCAGC |
| siRNA 1277 | 1277 | CUGCCCGGCUCGCACCCCU | 3328 | AGGGGUGCGAGCCGGGCAG |
| siRNA 1278 | 1278 | UGCCCGGCUCGCACCCCUG | 3329 | CAGGGGUGCGAGCCGGGCA |
| siRNA 1279 | 1279 | GCCCGGCUCGCACCCCUGG | 3330 | CCAGGGGUGCGAGCCGGGC |
| siRNA 1280 | 1280 | CCCGGCUCGCACCCCUGGC | 3331 | GCCAGGGGUGCGAGCCGGG |
| siRNA 1281 | 1281 | CCGGCUCGCACCCCUGGCU | 3332 | AGCCAGGGGUGCGAGCCGG |
| siRNA 1282 | 1282 | CGGCUCGCACCCCUGGCUG | 3333 | CAGCCAGGGGUGCGAGCCG |
| siRNA 1283 | 1283 | GGCUCGCACCCCUGGCUGG | 3334 | CCAGCCAGGGGUGCGAGCC |
| siRNA 1284 | 1284 | GCUCGCACCCCUGGCUGGC | 3335 | GCCAGCCAGGGGUGCGAGC |
| siRNA 1285 | 1285 | CUCGCACCCCUGGCUGGCC | 3336 | GGCCAGCCAGGGGUGCGAG |
| siRNA 1286 | 1286 | UCGCACCCCUGGCUGGCCG | 3337 | CGGCCAGCCAGGGGUGCGA |
| siRNA 1287 | 1287 | CGCACCCCUGGCUGGCCGC | 3338 | GCGGCCAGCCAGGGGUGCG |
| siRNA 1288 | 1288 | GCACCCCUGGCUGGCCGCC | 3339 | GGCGGCCAGCCAGGGGUGC |
| siRNA 1289 | 1289 | CACCCCUGGCUGGCCGCCA | 3340 | UGGCGGCCAGCCAGGGGUG |
| siRNA 1290 | 1290 | ACCCCUGGCUGGCCGCCAU | 3341 | AUGGCGGCCAGCCAGGGGU |
| siRNA 1291 | 1291 | CCCCUGGCUGGCCGCCAUC | 3342 | GAUGGCGGCCAGCCAGGGG |
| siRNA 1292 | 1292 | CCCUGGCUGGCCGCCAUCU | 3343 | AGAUGGCGGCCAGCCAGGG |
| siRNA 1293 | 1293 | CCUGGCUGGCCGCCAUCUA | 3344 | UAGAUGGCGGCCAGCCAGG |
| siRNA 1294 | 1294 | CUGGCUGGCCGCCAUCUAC | 3345 | GUAGAUGGCGGCCAGCCAG |
| siRNA 1295 | 1295 | UGGCUGGCCGCCAUCUACA | 3346 | UGUAGAUGGCGGCCAGCCA |
| siRNA 1296 | 1296 | GGCUGGCCGCCAUCUACAU | 3347 | AUGUAGAUGGCGGCCAGCC |
| siRNA 1297 | 1297 | GCUGGCCGCCAUCUACAUC | 3348 | GAUGUAGAUGGCGGCCAGC |
| siRNA 1298 | 1298 | CUGGCCGCCAUCUACAUCG | 3349 | CGAUGUAGAUGGCGGCCAG |
| siRNA 1299 | 1299 | UGGCCGCCAUCUACAUCGG | 3350 | CCGAUGUAGAUGGCGGCCA |
| siRNA 1300 | 1300 | GGCCGCCAUCUACAUCGGG | 3351 | CCCGAUGUAGAUGGCGGCC |
| siRNA 1301 | 1301 | GCCGCCAUCUACAUCGGGG | 3352 | CCCCGAUGUAGAUGGCGGC |
| siRNA 1302 | 1302 | CCGCCAUCUACAUCGGGGA | 3353 | UCCCCGAUGUAGAUGGCGG |
| siRNA 1303 | 1303 | CGCCAUCUACAUCGGGGAC | 3354 | GUCCCCGAUGUAGAUGGCG |
| siRNA 1304 | 1304 | GCCAUCUACAUCGGGGACA | 3355 | UGUCCCCGAUGUAGAUGGC |
| siRNA 1305 | 1305 | CCAUCUACAUCGGGGACAG | 3356 | CUGUCCCCGAUGUAGAUGG |
| siRNA 1306 | 1306 | CAUCUACAUCGGGGACAGC | 3357 | GCUGUCCCCGAUGUAGAUG |
| siRNA 1307 | 1307 | AUCUACAUCGGGGACAGCU | 3358 | AGCUGUCCCCGAUGUAGAU |
| siRNA 1308 | 1308 | UCUACAUCGGGGACAGCUU | 3359 | AAGCUGUCCCCGAUGUAGA |
| siRNA 1309 | 1309 | CUACAUCGGGGACAGCUUC | 3360 | GAAGCUGUCCCCGAUGUAG |
| siRNA 1310 | 1310 | UACAUCGGGGACAGCUUCU | 3361 | AGAAGCUGUCCCCGAUGUA |
| siRNA 1311 | 1311 | ACAUCGGGGACAGCUUCUG | 3362 | CAGAAGCUGUCCCCGAUGU |
| siRNA 1312 | 1312 | CAUCGGGGACAGCUUCUGC | 3363 | GCAGAAGCUGUCCCCGAUG |
| siRNA 1313 | 1313 | AUCGGGGACAGCUUCUGCG | 3364 | CGCAGAAGCUGUCCCCGAU |
| siRNA 1314 | 1314 | UCGGGGACAGCUUCUGCGC | 3365 | GCGCAGAAGCUGUCCCCGA |
| siRNA 1315 | 1315 | CGGGGACAGCUUCUGCGCC | 3366 | GGCGCAGAAGCUGUCCCCG |
| siRNA 1316 | 1316 | GGGGACAGCUUCUGCGCCG | 3367 | CGGCGCAGAAGCUGUCCCC |
| siRNA 1317 | 1317 | GGGACAGCUUCUGCGCCGG | 3368 | CCGGCGCAGAAGCUGUCCC |
| siRNA 1318 | 1318 | GGACAGCUUCUGCGCCGGG | 3369 | CCCGGCGCAGAAGCUGUCC |
| siRNA 1319 | 1319 | GACAGCUUCUGCGCCGGGA | 3370 | UCCCGGCGCAGAAGCUGUC |
| siRNA 1320 | 1320 | ACAGCUUCUGCGCCGGGAG | 3371 | CUCCCGGCGCAGAAGCUGU |
| siRNA 1321 | 1321 | CAGCUUCUGCGCCGGGAGC | 3372 | GCUCCCGGCGCAGAAGCUG |
| siRNA 1322 | 1322 | AGCUUCUGCGCCGGGAGCC | 3373 | GGCUCCCGGCGCAGAAGCU |
| siRNA 1323 | 1323 | GCUUCUGCGCCGGGAGCCU | 3374 | AGGCUCCCGGCGCAGAAGC |
| siRNA 1324 | 1324 | CUUCUGCGCCGGGAGCCUG | 3375 | CAGGCUCCCGGCGCAGAAG |
| siRNA 1325 | 1325 | UUCUGCGCCGGGAGCCUGG | 3376 | CCAGGCUCCCGGCGCAGAA |
| siRNA 1326 | 1326 | UCUGCGCCGGGAGCCUGGU | 3377 | ACCAGGCUCCCGGCGCAGA |
| siRNA 1327 | 1327 | CUGCGCCGGGAGCCUGGUC | 3378 | GACCAGGCUCCCGGCGCAG |
| siRNA 1328 | 1328 | UGCGCCGGGAGCCUGGUCC | 3379 | GGACCAGGCUCCCGGCGCA |
| SiRNA 1329 | 1329 | GCGCCGGGAGCCUGGUCCA | 3380 | UGGACCAGGCUCCCGGCGC |
| siRNA 1330 | 1330 | CGCCGGGAGCCUGGUCCAC | 3381 | GUGGACCAGGCUCCCGGCG |
| siRNA 1331 | 1331 | GCCGGGAGCCUGGUCCACA | 3382 | UGUGGACCAGGCUCCCGGC |
| siRNA 1332 | 1332 | CCGGGAGCCUGGUCCACAC | 3383 | GUGUGGACCAGGCUCCCGG |
| siRNA 1333 | 1333 | CGGGAGCCUGGUCCACACC | 3384 | GGUGUGGACCAGGCUCCCG |
| siRNA 1334 | 1334 | GGGAGCCUGGUCCACACCU | 3385 | AGGUGUGGACCAGGCUCCC |
| siRNA 1335 | 1335 | GGAGCCUGGUCCACACCUG | 3386 | CAGGUGUGGACCAGGCUCC |
| siRNA 1336 | 1336 | GAGCCUGGUCCACACCUGC | 3387 | GCAGGUGUGGACCAGGCUC |
| siRNA 1337 | 1337 | AGCCUGGUCCACACCUGCU | 3388 | AGCAGGUGUGGACCAGGCU |
| siRNA 1338 | 1338 | GCCUGGUCCACACCUGCUG | 3389 | CAGCAGGUGUGGACCAGGC |
| siRNA 1339 | 1339 | CCUGGUCCACACCUGCUGG | 3390 | CCAGCAGGUGUGGACCAGG |
| SiRNA 1340 | 1340 | CUGGUCCACACCUGCUGGG | 3391 | CCCAGCAGGUGUGGACCAG |
| siRNA 1341 | 1341 | UGGUCCACACCUGCUGGGU | 3392 | ACCCAGCAGGUGUGGACCA |
| siRNA 1342 | 1342 | GGUCCACACCUGCUGGGUG | 3393 | CACCCAGCAGGUGUGGACC |
| siRNA 1343 | 1343 | GUCCACACCUGCUGGGUGG | 3394 | CCACCCAGCAGGUGUGGAC |
| SiRNA 1344 | 1344 | UCCACACCUGCUGGGUGGU | 3395 | ACCACCCAGCAGGUGUGGA |
| siRNA 1345 | 1345 | CCACACCUGCUGGGUGGUG | 3396 | CACCACCCAGCAGGUGUGG |
| siRNA 1346 | 1346 | CACACCUGCUGGGUGGUGU | 3397 | ACACCACCCAGCAGGUGUG |
| siRNA 1347 | 1347 | ACACCUGCUGGGUGGUGUC | 3398 | GACACCACCCAGCAGGUGU |
| siRNA 1348 | 1348 | CACCUGCUGGGUGGUGUCG | 3399 | CGACACCACCCAGCAGGUG |
| SiRNA 1349 | 1349 | ACCUGCUGGGUGGUGUCGG | 3400 | CCGACACCACCCAGCAGGU |
| siRNA 1350 | 1350 | CCUGCUGGGUGGUGUCGGC | 3401 | GCCGACACCACCCAGCAGG |
| siRNA 1351 | 1351 | CUGCUGGGUGGUGUCGGCC | 3402 | GGCCGACACCACCCAGCAG |
| siRNA 1352 | 1352 | UGCUGGGUGGUGUCGGCCG | 3403 | CGGCCGACACCACCCAGCA |
| siRNA 1353 | 1353 | GCUGGGUGGUGUCGGCCGC | 3404 | GCGGCCGACACCACCCAGC |
| siRNA 1354 | 1354 | CUGGGUGGUGUCGGCCGCC | 3405 | GGCGGCCGACACCACCCAG |
| siRNA 1355 | 1355 | UGGGUGGUGUCGGCCGCCC | 3406 | GGGCGGCCGACACCACCCA |
| siRNA 1356 | 1356 | GGGUGGUGUCGGCCGCCCA | 3407 | UGGGCGGCCGACACCACCC |
| siRNA 1357 | 1357 | GGUGGUGUCGGCCGCCCAC | 3408 | GUGGGCGGCCGACACCACC |
| siRNA 1358 | 1358 | GUGGUGUCGGCCGCCCACU | 3409 | AGUGGGCGGCCGACACCAC |
| siRNA 1359 | 1359 | UGGUGUCGGCCGCCCACUG | 3410 | CAGUGGGCGGCCGACACCA |
| siRNA 1360 | 1360 | GGUGUCGGCCGCCCACUGC | 3411 | GCAGUGGGCGGCCGACACC |
| siRNA 1361 | 1361 | GUGUCGGCCGCCCACUGCU | 3412 | AGCAGUGGGCGGCCGACAC |
| siRNA 1362 | 1362 | UGUCGGCCGCCCACUGCUU | 3413 | AAGCAGUGGGCGGCCGACA |
| siRNA 1363 | 1363 | GUCGGCCGCCCACUGCUUC | 3414 | GAAGCAGUGGGCGGCCGAC |
| siRNA 1364 | 1364 | UCGGCCGCCCACUGCUUCU | 3415 | AGAAGCAGUGGGCGGCCGA |
| siRNA 1365 | 1365 | CGGCCGCCCACUGCUUCUC | 3416 | GAGAAGCAGUGGGCGGCCG |
| siRNA 1366 | 1366 | GGCCGCCCACUGCUUCUCC | 3417 | GGAGAAGCAGUGGGCGGCC |
| siRNA 1367 | 1367 | GCCGCCCACUGCUUCUCCC | 3418 | GGGAGAAGCAGUGGGCGGC |
| siRNA 1368 | 1368 | CCGCCCACUGCUUCUCCCA | 3419 | UGGGAGAAGCAGUGGGCGG |
| siRNA 1369 | 1369 | CGCCCACUGCUUCUCCCAC | 3420 | GUGGGAGAAGCAGUGGGCG |
| siRNA 1370 | 1370 | GCCCACUGCUUCUCCCACA | 3421 | UGUGGGAGAAGCAGUGGGC |
| SiRNA 1371 | 1371 | CCCACUGCUUCUCCCACAG | 3422 | CUGUGGGAGAAGCAGUGGG |
| siRNA 1372 | 1372 | CCACUGCUUCUCCCACAGC | 3423 | GCUGUGGGAGAAGCAGUGG |
| siRNA 1373 | 1373 | CACUGCUUCUCCCACAGCC | 3424 | GGCUGUGGGAGAAGCAGUG |
| SiRNA 1374 | 1374 | ACUGCUUCUCCCACAGCCC | 3425 | GGGCUGUGGGAGAAGCAGU |
| siRNA 1375 | 1375 | CUGCUUCUCCCACAGCCCC | 3426 | GGGGCUGUGGGAGAAGCAG |
| siRNA 1376 | 1376 | UGCUUCUCCCACAGCCCCC | 3427 | GGGGGCUGUGGGAGAAGCA |
| siRNA 1377 | 1377 | GCUUCUCCCACAGCCCCCC | 3428 | GGGGGGCUGUGGGAGAAGC |
| siRNA 1378 | 1378 | CUUCUCCCACAGCCCCCCC | 3429 | GGGGGGGCUGUGGGAGAAG |
| siRNA 1379 | 1379 | UUCUCCCACAGCCCCCCCA | 3430 | UGGGGGGGCUGUGGGAGAA |
| siRNA 1380 | 1380 | UCUCCCACAGCCCCCCCAG | 3431 | CUGGGGGGGCUGUGGGAGA |
| siRNA 1381 | 1381 | CUCCCACAGCCCCCCCAGG | 3432 | CCUGGGGGGGCUGUGGGAG |
| siRNA 1382 | 1382 | UCCCACAGCCCCCCCAGGG | 3433 | CCCUGGGGGGGCUGUGGGA |
| siRNA 1383 | 1383 | CCCACAGCCCCCCCAGGGA | 3434 | UCCCUGGGGGGGCUGUGGG |
| siRNA 1384 | 1384 | CCACAGCCCCCCCAGGGAC | 3435 | GUCCCUGGGGGGGCUGUGG |
| siRNA 1385 | 1385 | CACAGCCCCCCCAGGGACA | 3436 | UGUCCCUGGGGGGGCUGUG |
| siRNA 1386 | 1386 | ACAGCCCCCCCAGGGACAG | 3437 | CUGUCCCUGGGGGGGCUGU |
| siRNA 1387 | 1387 | CAGCCCCCCCAGGGACAGC | 3438 | GCUGUCCCUGGGGGGGCUG |
| siRNA 1388 | 1388 | AGCCCCCCCAGGGACAGCG | 3439 | CGCUGUCCCUGGGGGGGCU |
| SiRNA 1389 | 1389 | GCCCCCCCAGGGACAGCGU | 3440 | ACGCUGUCCCUGGGGGGGC |
| siRNA 1390 | 1390 | CCCCCCCAGGGACAGCGUC | 3441 | GACGCUGUCCCUGGGGGGG |
| siRNA 1391 | 1391 | CCCCCCAGGGACAGCGUCU | 3442 | AGACGCUGUCCCUGGGGGG |
| siRNA 1392 | 1392 | CCCCCAGGGACAGCGUCUC | 3443 | GAGACGCUGUCCCUGGGGG |
| siRNA 1393 | 1393 | CCCCAGGGACAGCGUCUCC | 3444 | GGAGACGCUGUCCCUGGGG |
| siRNA 1394 | 1394 | CCCAGGGACAGCGUCUCCG | 3445 | CGGAGACGCUGUCCCUGGG |
| siRNA 1395 | 1395 | CCAGGGACAGCGUCUCCGU | 3446 | ACGGAGACGCUGUCCCUGG |
| siRNA 1396 | 1396 | CAGGGACAGCGUCUCCGUG | 3447 | CACGGAGACGCUGUCCCUG |
| siRNA 1397 | 1397 | AGGGACAGCGUCUCCGUGG | 3448 | CCACGGAGACGCUGUCCCU |
| siRNA 1398 | 1398 | GGGACAGCGUCUCCGUGGU | 3449 | ACCACGGAGACGCUGUCCC |
| SiRNA 1399 | 1399 | GGACAGCGUCUCCGUGGUG | 3450 | CACCACGGAGACGCUGUCC |
| siRNA 1400 | 1400 | GACAGCGUCUCCGUGGUGC | 3451 | GCACCACGGAGACGCUGUC |
| siRNA 1401 | 1401 | ACAGCGUCUCCGUGGUGCU | 3452 | AGCACCACGGAGACGCUGU |
| siRNA 1402 | 1402 | CAGCGUCUCCGUGGUGCUG | 3453 | CAGCACCACGGAGACGCUG |
| siRNA 1403 | 1403 | AGCGUCUCCGUGGUGCUGG | 3454 | CCAGCACCACGGAGACGCU |
| siRNA 1404 | 1404 | GCGUCUCCGUGGUGCUGGG | 3455 | CCCAGCACCACGGAGACGC |
| siRNA 1405 | 1405 | CGUCUCCGUGGUGCUGGGC | 3456 | GCCCAGCACCACGGAGACG |
| siRNA 1406 | 1406 | GUCUCCGUGGUGCUGGGCC | 3457 | GGCCCAGCACCACGGAGAC |
| siRNA 1407 | 1407 | UCUCCGUGGUGCUGGGCCA | 3458 | UGGCCCAGCACCACGGAGA |
| SiRNA 1408 | 1408 | CUCCGUGGUGCUGGGCCAG | 3459 | CUGGCCCAGCACCACGGAG |
| siRNA 1409 | 1409 | UCCGUGGUGCUGGGCCAGC | 3460 | GCUGGCCCAGCACCACGGA |
| siRNA 1410 | 1410 | CCGUGGUGCUGGGCCAGCA | 3461 | UGCUGGCCCAGCACCACGG |
| siRNA 1411 | 1411 | CGUGGUGCUGGGCCAGCAC | 3462 | GUGCUGGCCCAGCACCACG |
| siRNA 1412 | 1412 | GUGGUGCUGGGCCAGCACU | 3463 | AGUGCUGGCCCAGCACCAC |
| siRNA 1413 | 1413 | UGGUGCUGGGCCAGCACUU | 3464 | AAGUGCUGGCCCAGCACCA |
| SiRNA 1414 | 1414 | GGUGCUGGGCCAGCACUUC | 3465 | GAAGUGCUGGCCCAGCACC |
| siRNA 1415 | 1415 | GUGCUGGGCCAGCACUUCU | 3466 | AGAAGUGCUGGCCCAGCAC |
| siRNA 1416 | 1416 | UGCUGGGCCAGCACUUCUU | 3467 | AAGAAGUGCUGGCCCAGCA |
| siRNA 1417 | 1417 | GCUGGGCCAGCACUUCUUC | 3468 | GAAGAAGUGCUGGCCCAGC |
| siRNA 1418 | 1418 | CUGGGCCAGCACUUCUUCA | 3469 | UGAAGAAGUGCUGGCCCAG |
| siRNA 1419 | 1419 | UGGGCCAGCACUUCUUCAA | 3470 | UUGAAGAAGUGCUGGCCCA |
| siRNA 1420 | 1420 | GGGCCAGCACUUCUUCAAC | 3471 | GUUGAAGAAGUGCUGGCCC |
| siRNA 1421 | 1421 | GGCCAGCACUUCUUCAACC | 3472 | GGUUGAAGAAGUGCUGGCC |
| siRNA 1422 | 1422 | GCCAGCACUUCUUCAACCG | 3473 | CGGUUGAAGAAGUGCUGGC |
| siRNA 1423 | 1423 | CCAGCACUUCUUCAACCGC | 3474 | GCGGUUGAAGAAGUGCUGG |
| siRNA 1424 | 1424 | CAGCACUUCUUCAACCGCA | 3475 | UGCGGUUGAAGAAGUGCUG |
| siRNA 1425 | 1425 | AGCACUUCUUCAACCGCAC | 3476 | GUGCGGUUGAAGAAGUGCU |
| siRNA 1426 | 1426 | GCACUUCUUCAACCGCACG | 3477 | CGUGCGGUUGAAGAAGUGC |
| siRNA 1427 | 1427 | CACUUCUUCAACCGCACGA | 3478 | UCGUGCGGUUGAAGAAGUG |
| siRNA 1428 | 1428 | ACUUCUUCAACCGCACGAC | 3479 | GUCGUGCGGUUGAAGAAGU |
| siRNA 1429 | 1429 | CUUCUUCAACCGCACGACG | 3480 | CGUCGUGCGGUUGAAGAAG |
| siRNA 1430 | 1430 | UUCUUCAACCGCACGACGG | 3481 | CCGUCGUGCGGUUGAAGAA |
| siRNA 1431 | 1431 | UCUUCAACCGCACGACGGA | 3482 | UCCGUCGUGCGGUUGAAGA |
| siRNA 1432 | 1432 | CUUCAACCGCACGACGGAC | 3483 | GUCCGUCGUGCGGUUGAAG |
| siRNA 1433 | 1433 | UUCAACCGCACGACGGACG | 3484 | CGUCCGUCGUGCGGUUGAA |
| SiRNA 1434 | 1434 | UCAACCGCACGACGGACGU | 3485 | ACGUCCGUCGUGCGGUUGA |
| siRNA 1435 | 1435 | CAACCGCACGACGGACGUG | 3486 | CACGUCCGUCGUGCGGUUG |
| siRNA 1436 | 1436 | AACCGCACGACGGACGUGA | 3487 | UCACGUCCGUCGUGCGGUU |
| siRNA 1437 | 1437 | ACCGCACGACGGACGUGAC | 3488 | GUCACGUCCGUCGUGCGGU |
| siRNA 1438 | 1438 | CCGCACGACGGACGUGACG | 3489 | CGUCACGUCCGUCGUGCGG |
| siRNA 1439 | 1439 | CGCACGACGGACGUGACGC | 3490 | GCGUCACGUCCGUCGUGCG |
| siRNA 1440 | 1440 | GCACGACGGACGUGACGCA | 3491 | UGCGUCACGUCCGUCGUGC |
| siRNA 1441 | 1441 | CACGACGGACGUGACGCAG | 3492 | CUGCGUCACGUCCGUCGUG |
| siRNA 1442 | 1442 | ACGACGGACGUGACGCAGA | 3493 | UCUGCGUCACGUCCGUCGU |
| siRNA 1443 | 1443 | CGACGGACGUGACGCAGAC | 3494 | GUCUGCGUCACGUCCGUCG |
| siRNA 1444 | 1444 | GACGGACGUGACGCAGACC | 3495 | GGUCUGCGUCACGUCCGUC |
| siRNA 1445 | 1445 | ACGGACGUGACGCAGACCU | 3496 | AGGUCUGCGUCACGUCCGU |
| siRNA 1446 | 1446 | CGGACGUGACGCAGACCUU | 3497 | AAGGUCUGCGUCACGUCCG |
| siRNA 1447 | 1447 | GGACGUGACGCAGACCUUC | 3498 | GAAGGUCUGCGUCACGUCC |
| siRNA 1448 | 1448 | GACGUGACGCAGACCUUCG | 3499 | CGAAGGUCUGCGUCACGUC |
| siRNA 1449 | 1449 | ACGUGACGCAGACCUUCGG | 3500 | CCGAAGGUCUGCGUCACGU |
| SiRNA 1450 | 1450 | CGUGACGCAGACCUUCGGC | 3501 | GCCGAAGGUCUGCGUCACG |
| siRNA 1451 | 1451 | GUGACGCAGACCUUCGGCA | 3502 | UGCCGAAGGUCUGCGUCAC |
| siRNA 1452 | 1452 | UGACGCAGACCUUCGGCAU | 3503 | AUGCCGAAGGUCUGCGUCA |
| SiRNA 1453 | 1453 | GACGCAGACCUUCGGCAUC | 3504 | GAUGCCGAAGGUCUGCGUC |
| siRNA 1454 | 1454 | ACGCAGACCUUCGGCAUCG | 3505 | CGAUGCCGAAGGUCUGCGU |
| siRNA 1455 | 1455 | CGCAGACCUUCGGCAUCGA | 3506 | UCGAUGCCGAAGGUCUGCG |
| siRNA 1456 | 1456 | GCAGACCUUCGGCAUCGAG | 3507 | CUCGAUGCCGAAGGUCUGC |
| siRNA 1457 | 1457 | CAGACCUUCGGCAUCGAGA | 3508 | UCUCGAUGCCGAAGGUCUG |
| siRNA 1458 | 1458 | AGACCUUCGGCAUCGAGAA | 3509 | UUCUCGAUGCCGAAGGUCU |
| SiRNA 1459 | 1459 | GACCUUCGGCAUCGAGAAG | 3510 | CUUCUCGAUGCCGAAGGUC |
| siRNA 1460 | 1460 | ACCUUCGGCAUCGAGAAGU | 3511 | ACUUCUCGAUGCCGAAGGU |
| siRNA 1461 | 1461 | CCUUCGGCAUCGAGAAGUA | 3512 | UACUUCUCGAUGCCGAAGG |
| siRNA 1462 | 1462 | CUUCGGCAUCGAGAAGUAC | 3513 | GUACUUCUCGAUGCCGAAG |
| siRNA 1463 | 1463 | UUCGGCAUCGAGAAGUACA | 3514 | UGUACUUCUCGAUGCCGAA |
| siRNA 1464 | 1464 | UCGGCAUCGAGAAGUACAU | 3515 | AUGUACUUCUCGAUGCCGA |
| siRNA 1465 | 1465 | CGGCAUCGAGAAGUACAUC | 3516 | GAUGUACUUCUCGAUGCCG |
| siRNA 1466 | 1466 | GGCAUCGAGAAGUACAUCC | 3517 | GGAUGUACUUCUCGAUGCC |
| siRNA 1467 | 1467 | GCAUCGAGAAGUACAUCCC | 3518 | GGGAUGUACUUCUCGAUGC |
| siRNA 1468 | 1468 | CAUCGAGAAGUACAUCCCG | 3519 | CGGGAUGUACUUCUCGAUG |
| siRNA 1469 | 1469 | AUCGAGAAGUACAUCCCGU | 3520 | ACGGGAUGUACUUCUCGAU |
| siRNA 1470 | 1470 | UCGAGAAGUACAUCCCGUA | 3521 | UACGGGAUGUACUUCUCGA |
| siRNA 1471 | 1471 | CGAGAAGUACAUCCCGUAC | 3522 | GUACGGGAUGUACUUCUCG |
| siRNA 1472 | 1472 | GAGAAGUACAUCCCGUACA | 3523 | UGUACGGGAUGUACUUCUC |
| siRNA 1473 | 1473 | AGAAGUACAUCCCGUACAC | 3524 | GUGUACGGGAUGUACUUCU |
| siRNA 1474 | 1474 | GAAGUACAUCCCGUACACC | 3525 | GGUGUACGGGAUGUACUUC |
| siRNA 1475 | 1475 | AAGUACAUCCCGUACACCC | 3526 | GGGUGUACGGGAUGUACUU |
| siRNA 1476 | 1476 | AGUACAUCCCGUACACCCU | 3527 | AGGGUGUACGGGAUGUACU |
| siRNA 1477 | 1477 | GUACAUCCCGUACACCCUG | 3528 | CAGGGUGUACGGGAUGUAC |
| siRNA 1478 | 1478 | UACAUCCCGUACACCCUGU | 3529 | ACAGGGUGUACGGGAUGUA |
| siRNA 1479 | 1479 | ACAUCCCGUACACCCUGUA | 3530 | UACAGGGUGUACGGGAUGU |
| SiRNA 1480 | 1480 | CAUCCCGUACACCCUGUAC | 3531 | GUACAGGGUGUACGGGAUG |
| siRNA 1481 | 1481 | AUCCCGUACACCCUGUACU | 3532 | AGUACAGGGUGUACGGGAU |
| SiRNA 1482 | 1482 | UCCCGUACACCCUGUACUC | 3533 | GAGUACAGGGUGUACGGGA |
| siRNA 1483 | 1483 | CCCGUACACCCUGUACUCG | 3534 | CGAGUACAGGGUGUACGGG |
| SiRNA 1484 | 1484 | CCGUACACCCUGUACUCGG | 3535 | CCGAGUACAGGGUGUACGG |
| siRNA 1485 | 1485 | CGUACACCCUGUACUCGGU | 3536 | ACCGAGUACAGGGUGUACG |
| siRNA 1486 | 1486 | GUACACCCUGUACUCGGUG | 3537 | CACCGAGUACAGGGUGUAC |
| siRNA 1487 | 1487 | UACACCCUGUACUCGGUGU | 3538 | ACACCGAGUACAGGGUGUA |
| siRNA 1488 | 1488 | ACACCCUGUACUCGGUGUU | 3539 | AACACCGAGUACAGGGUGU |
| SiRNA 1489 | 1489 | CACCCUGUACUCGGUGUUC | 3540 | GAACACCGAGUACAGGGUG |
| siRNA 1490 | 1490 | ACCCUGUACUCGGUGUUCA | 3541 | UGAACACCGAGUACAGGGU |
| siRNA 1491 | 1491 | CCCUGUACUCGGUGUUCAA | 3542 | UUGAACACCGAGUACAGGG |
| siRNA 1492 | 1492 | CCUGUACUCGGUGUUCAAC | 3543 | GUUGAACACCGAGUACAGG |
| siRNA 1493 | 1493 | CUGUACUCGGUGUUCAACC | 3544 | GGUUGAACACCGAGUACAG |
| siRNA 1494 | 1494 | UGUACUCGGUGUUCAACCC | 3545 | GGGUUGAACACCGAGUACA |
| siRNA 1495 | 1495 | GUACUCGGUGUUCAACCCC | 3546 | GGGGUUGAACACCGAGUAC |
| siRNA 1496 | 1496 | UACUCGGUGUUCAACCCCA | 3547 | UGGGGUUGAACACCGAGUA |
| siRNA 1497 | 1497 | ACUCGGUGUUCAACCCCAG | 3548 | CUGGGGUUGAACACCGAGU |
| siRNA 1498 | 1498 | CUCGGUGUUCAACCCCAGC | 3549 | GCUGGGGUUGAACACCGAG |
| siRNA 1499 | 1499 | UCGGUGUUCAACCCCAGCG | 3550 | CGCUGGGGUUGAACACCGA |
| siRNA 1500 | 1500 | CGGUGUUCAACCCCAGCGA | 3551 | UCGCUGGGGUUGAACACCG |
| siRNA 1501 | 1501 | GGUGUUCAACCCCAGCGAC | 3552 | GUCGCUGGGGUUGAACACC |
| siRNA 1502 | 1502 | GUGUUCAACCCCAGCGACC | 3553 | GGUCGCUGGGGUUGAACAC |
| siRNA 1503 | 1503 | UGUUCAACCCCAGCGACCA | 3554 | UGGUCGCUGGGGUUGAACA |
| siRNA 1504 | 1504 | GUUCAACCCCAGCGACCAC | 3555 | GUGGUCGCUGGGGUUGAAC |
| siRNA 1505 | 1505 | UUCAACCCCAGCGACCACG | 3556 | CGUGGUCGCUGGGGUUGAA |
| siRNA 1506 | 1506 | UCAACCCCAGCGACCACGA | 3557 | UCGUGGUCGCUGGGGUUGA |
| siRNA 1507 | 1507 | CAACCCCAGCGACCACGAC | 3558 | GUCGUGGUCGCUGGGGUUG |
| siRNA 1508 | 1508 | AACCCCAGCGACCACGACC | 3559 | GGUCGUGGUCGCUGGGGUU |
| siRNA 1509 | 1509 | ACCCCAGCGACCACGACCU | 3560 | AGGUCGUGGUCGCUGGGGU |
| siRNA 1510 | 1510 | CCCCAGCGACCACGACCUC | 3561 | GAGGUCGUGGUCGCUGGGG |
| siRNA 1511 | 1511 | CCCAGCGACCACGACCUCG | 3562 | CGAGGUCGUGGUCGCUGGG |
| siRNA 1512 | 1512 | CCAGCGACCACGACCUCGU | 3563 | ACGAGGUCGUGGUCGCUGG |
| siRNA 1513 | 1513 | CAGCGACCACGACCUCGUC | 3564 | GACGAGGUCGUGGUCGCUG |
| siRNA 1514 | 1514 | AGCGACCACGACCUCGUCC | 3565 | GGACGAGGUCGUGGUCGCU |
| siRNA 1515 | 1515 | GCGACCACGACCUCGUCCU | 3566 | AGGACGAGGUCGUGGUCGC |
| siRNA 1516 | 1516 | CGACCACGACCUCGUCCUG | 3567 | CAGGACGAGGUCGUGGUCG |
| siRNA 1517 | 1517 | GACCACGACCUCGUCCUGA | 3568 | UCAGGACGAGGUCGUGGUC |
| siRNA 1518 | 1518 | ACCACGACCUCGUCCUGAU | 3569 | AUCAGGACGAGGUCGUGGU |
| siRNA 1519 | 1519 | CCACGACCUCGUCCUGAUC | 3570 | GAUCAGGACGAGGUCGUGG |
| siRNA 1520 | 1520 | CACGACCUCGUCCUGAUCC | 3571 | GGAUCAGGACGAGGUCGUG |
| siRNA 1521 | 1521 | ACGACCUCGUCCUGAUCCG | 3572 | CGGAUCAGGACGAGGUCGU |
| siRNA 1522 | 1522 | CGACCUCGUCCUGAUCCGG | 3573 | CCGGAUCAGGACGAGGUCG |
| siRNA 1523 | 1523 | GACCUCGUCCUGAUCCGGC | 3574 | GCCGGAUCAGGACGAGGUC |
| siRNA 1524 | 1524 | ACCUCGUCCUGAUCCGGCU | 3575 | AGCCGGAUCAGGACGAGGU |
| siRNA 1525 | 1525 | CCUCGUCCUGAUCCGGCUG | 3576 | CAGCCGGAUCAGGACGAGG |
| siRNA 1526 | 1526 | CUCGUCCUGAUCCGGCUGA | 3577 | UCAGCCGGAUCAGGACGAG |
| siRNA 1527 | 1527 | UCGUCCUGAUCCGGCUGAA | 3578 | UUCAGCCGGAUCAGGACGA |
| siRNA 1528 | 1528 | CGUCCUGAUCCGGCUGAAG | 3579 | CUUCAGCCGGAUCAGGACG |
| siRNA 1529 | 1529 | GUCCUGAUCCGGCUGAAGA | 3580 | UCUUCAGCCGGAUCAGGAC |
| siRNA 1530 | 1530 | UCCUGAUCCGGCUGAAGAA | 3581 | UUCUUCAGCCGGAUCAGGA |
| siRNA 1531 | 1531 | CCUGAUCCGGCUGAAGAAG | 3582 | CUUCUUCAGCCGGAUCAGG |
| siRNA 1532 | 1532 | CUGAUCCGGCUGAAGAAGA | 3583 | UCUUCUUCAGCCGGAUCAG |
| siRNA 1533 | 1533 | UGAUCCGGCUGAAGAAGAA | 3584 | UUCUUCUUCAGCCGGAUCA |
| siRNA 1534 | 1534 | GAUCCGGCUGAAGAAGAAA | 3585 | UUUCUUCUUCAGCCGGAUC |
| siRNA 1535 | 1535 | AUCCGGCUGAAGAAGAAAG | 3586 | CUUUCUUCUUCAGCCGGAU |
| siRNA 1536 | 1536 | UCCGGCUGAAGAAGAAAGG | 3587 | CCUUUCUUCUUCAGCCGGA |
| siRNA 1537 | 1537 | CCGGCUGAAGAAGAAAGGG | 3588 | CCCUUUCUUCUUCAGCCGG |
| siRNA 1538 | 1538 | CGGCUGAAGAAGAAAGGGG | 3589 | CCCCUUUCUUCUUCAGCCG |
| siRNA 1539 | 1539 | GGCUGAAGAAGAAAGGGGA | 3590 | UCCCCUUUCUUCUUCAGCC |
| siRNA 1540 | 1540 | GCUGAAGAAGAAAGGGGAC | 3591 | GUCCCCUUUCUUCUUCAGC |
| siRNA 1541 | 1541 | CUGAAGAAGAAAGGGGACC | 3592 | GGUCCCCUUUCUUCUUCAG |
| siRNA 1542 | 1542 | UGAAGAAGAAAGGGGACCG | 3593 | CGGUCCCCUUUCUUCUUCA |
| siRNA 1543 | 1543 | GAAGAAGAAAGGGGACCGC | 3594 | GCGGUCCCCUUUCUUCUUC |
| siRNA 1544 | 1544 | AAGAAGAAAGGGGACCGCU | 3595 | AGCGGUCCCCUUUCUUCUU |
| siRNA 1545 | 1545 | AGAAGAAAGGGGACCGCUG | 3596 | CAGCGGUCCCCUUUCUUCU |
| siRNA 1546 | 1546 | GAAGAAAGGGGACCGCUGU | 3597 | ACAGCGGUCCCCUUUCUUC |
| siRNA 1547 | 1547 | AAGAAAGGGGACCGCUGUG | 3598 | CACAGCGGUCCCCUUUCUU |
| siRNA 1548 | 1548 | AGAAAGGGGACCGCUGUGC | 3599 | GCACAGCGGUCCCCUUUCU |
| siRNA 1549 | 1549 | GAAAGGGGACCGCUGUGCC | 3600 | GGCACAGCGGUCCCCUUUC |
| siRNA 1550 | 1550 | AAAGGGGACCGCUGUGCCA | 3601 | UGGCACAGCGGUCCCCUUU |
| siRNA 1551 | 1551 | AAGGGGACCGCUGUGCCAC | 3602 | GUGGCACAGCGGUCCCCUU |
| siRNA 1552 | 1552 | AGGGGACCGCUGUGCCACA | 3603 | UGUGGCACAGCGGUCCCCU |
| siRNA 1553 | 1553 | GGGGACCGCUGUGCCACAC | 3604 | GUGUGGCACAGCGGUCCCC |
| siRNA 1554 | 1554 | GGGACCGCUGUGCCACACG | 3605 | CGUGUGGCACAGCGGUCCC |
| siRNA 1555 | 1555 | GGACCGCUGUGCCACACGC | 3606 | GCGUGUGGCACAGCGGUCC |
| siRNA 1556 | 1556 | GACCGCUGUGCCACACGCU | 3607 | AGCGUGUGGCACAGCGGUC |
| siRNA 1557 | 1557 | ACCGCUGUGCCACACGCUC | 3608 | GAGCGUGUGGCACAGCGGU |
| siRNA 1558 | 1558 | CCGCUGUGCCACACGCUCG | 3609 | CGAGCGUGUGGCACAGCGG |
| siRNA 1559 | 1559 | CGCUGUGCCACACGCUCGC | 3610 | GCGAGCGUGUGGCACAGCG |
| siRNA 1560 | 1560 | GCUGUGCCACACGCUCGCA | 3611 | UGCGAGCGUGUGGCACAGC |
| siRNA 1561 | 1561 | CUGUGCCACACGCUCGCAG | 3612 | CUGCGAGCGUGUGGCACAG |
| siRNA 1562 | 1562 | UGUGCCACACGCUCGCAGU | 3613 | ACUGCGAGCGUGUGGCACA |
| siRNA 1563 | 1563 | GUGCCACACGCUCGCAGUU | 3614 | AACUGCGAGCGUGUGGCAC |
| siRNA 1564 | 1564 | UGCCACACGCUCGCAGUUC | 3615 | GAACUGCGAGCGUGUGGCA |
| siRNA 1565 | 1565 | GCCACACGCUCGCAGUUCG | 3616 | CGAACUGCGAGCGUGUGGC |
| siRNA 1566 | 1566 | CCACACGCUCGCAGUUCGU | 3617 | ACGAACUGCGAGCGUGUGG |
| siRNA 1567 | 1567 | CACACGCUCGCAGUUCGUG | 3618 | CACGAACUGCGAGCGUGUG |
| siRNA 1568 | 1568 | ACACGCUCGCAGUUCGUGC | 3619 | GCACGAACUGCGAGCGUGU |
| siRNA 1569 | 1569 | CACGCUCGCAGUUCGUGCA | 3620 | UGCACGAACUGCGAGCGUG |
| SiRNA 1570 | 1570 | ACGCUCGCAGUUCGUGCAG | 3621 | CUGCACGAACUGCGAGCGU |
| siRNA 1571 | 1571 | CGCUCGCAGUUCGUGCAGC | 3622 | GCUGCACGAACUGCGAGCG |
| siRNA 1572 | 1572 | GCUCGCAGUUCGUGCAGCC | 3623 | GGCUGCACGAACUGCGAGC |
| siRNA 1573 | 1573 | CUCGCAGUUCGUGCAGCCC | 3624 | GGGCUGCACGAACUGCGAG |
| siRNA 1574 | 1574 | UCGCAGUUCGUGCAGCCCA | 3625 | UGGGCUGCACGAACUGCGA |
| siRNA 1575 | 1575 | CGCAGUUCGUGCAGCCCAU | 3626 | AUGGGCUGCACGAACUGCG |
| siRNA 1576 | 1576 | GCAGUUCGUGCAGCCCAUC | 3627 | GAUGGGCUGCACGAACUGC |
| siRNA 1577 | 1577 | CAGUUCGUGCAGCCCAUCU | 3628 | AGAUGGGCUGCACGAACUG |
| siRNA 1578 | 1578 | AGUUCGUGCAGCCCAUCUG | 3629 | CAGAUGGGCUGCACGAACU |
| siRNA 1579 | 1579 | GUUCGUGCAGCCCAUCUGC | 3630 | GCAGAUGGGCUGCACGAAC |
| siRNA 1580 | 1580 | UUCGUGCAGCCCAUCUGCC | 3631 | GGCAGAUGGGCUGCACGAA |
| siRNA 1581 | 1581 | UCGUGCAGCCCAUCUGCCU | 3632 | AGGCAGAUGGGCUGCACGA |
| siRNA 1582 | 1582 | CGUGCAGCCCAUCUGCCUG | 3633 | CAGGCAGAUGGGCUGCACG |
| siRNA 1583 | 1583 | GUGCAGCCCAUCUGCCUGC | 3634 | GCAGGCAGAUGGGCUGCAC |
| siRNA 1584 | 1584 | UGCAGCCCAUCUGCCUGCC | 3635 | GGCAGGCAGAUGGGCUGCA |
| siRNA 1585 | 1585 | GCAGCCCAUCUGCCUGCCC | 3636 | GGGCAGGCAGAUGGGCUGC |
| siRNA 1586 | 1586 | CAGCCCAUCUGCCUGCCCG | 3637 | CGGGCAGGCAGAUGGGCUG |
| siRNA 1587 | 1587 | AGCCCAUCUGCCUGCCCGA | 3638 | UCGGGCAGGCAGAUGGGCU |
| siRNA 1588 | 1588 | GCCCAUCUGCCUGCCCGAG | 3639 | CUCGGGCAGGCAGAUGGGC |
| siRNA 1589 | 1589 | CCCAUCUGCCUGCCCGAGC | 3640 | GCUCGGGCAGGCAGAUGGG |
| siRNA 1590 | 1590 | CCAUCUGCCUGCCCGAGCC | 3641 | GGCUCGGGCAGGCAGAUGG |
| siRNA 1591 | 1591 | CAUCUGCCUGCCCGAGCCC | 3642 | GGGCUCGGGCAGGCAGAUG |
| siRNA 1592 | 1592 | AUCUGCCUGCCCGAGCCCG | 3643 | CGGGCUCGGGCAGGCAGAU |
| siRNA 1593 | 1593 | UCUGCCUGCCCGAGCCCGG | 3644 | CCGGGCUCGGGCAGGCAGA |
| siRNA 1594 | 1594 | CUGCCUGCCCGAGCCCGGC | 3645 | GCCGGGCUCGGGCAGGCAG |
| siRNA 1595 | 1595 | UGCCUGCCCGAGCCCGGCA | 3646 | UGCCGGGCUCGGGCAGGCA |
| siRNA 1596 | 1596 | GCCUGCCCGAGCCCGGCAG | 3647 | CUGCCGGGCUCGGGCAGGC |
| siRNA 1597 | 1597 | CCUGCCCGAGCCCGGCAGC | 3648 | GCUGCCGGGCUCGGGCAGG |
| siRNA 1598 | 1598 | CUGCCCGAGCCCGGCAGCA | 3649 | UGCUGCCGGGCUCGGGCAG |
| siRNA 1599 | 1599 | UGCCCGAGCCCGGCAGCAC | 3650 | GUGCUGCCGGGCUCGGGCA |
| siRNA 1600 | 1600 | GCCCGAGCCCGGCAGCACC | 3651 | GGUGCUGCCGGGCUCGGGC |
| siRNA 1601 | 1601 | CCCGAGCCCGGCAGCACCU | 3652 | AGGUGCUGCCGGGCUCGGG |
| siRNA 1602 | 1602 | CCGAGCCCGGCAGCACCUU | 3653 | AAGGUGCUGCCGGGCUCGG |
| siRNA 1603 | 1603 | CGAGCCCGGCAGCACCUUC | 3654 | GAAGGUGCUGCCGGGCUCG |
| siRNA 1604 | 1604 | GAGCCCGGCAGCACCUUCC | 3655 | GGAAGGUGCUGCCGGGCUC |
| siRNA 1605 | 1605 | AGCCCGGCAGCACCUUCCC | 3656 | GGGAAGGUGCUGCCGGGCU |
| siRNA 1606 | 1606 | GCCCGGCAGCACCUUCCCC | 3657 | GGGGAAGGUGCUGCCGGGC |
| siRNA 1607 | 1607 | CCCGGCAGCACCUUCCCCG | 3658 | CGGGGAAGGUGCUGCCGGG |
| siRNA 1608 | 1608 | CCGGCAGCACCUUCCCCGC | 3659 | GCGGGGAAGGUGCUGCCGG |
| siRNA 1609 | 1609 | CGGCAGCACCUUCCCCGCA | 3660 | UGCGGGGAAGGUGCUGCCG |
| siRNA 1610 | 1610 | GGCAGCACCUUCCCCGCAG | 3661 | CUGCGGGGAAGGUGCUGCC |
| siRNA 1611 | 1611 | GCAGCACCUUCCCCGCAGG | 3662 | CCUGCGGGGAAGGUGCUGC |
| siRNA 1612 | 1612 | CAGCACCUUCCCCGCAGGA | 3663 | UCCUGCGGGGAAGGUGCUG |
| siRNA 1613 | 1613 | AGCACCUUCCCCGCAGGAC | 3664 | GUCCUGCGGGGAAGGUGCU |
| siRNA 1614 | 1614 | GCACCUUCCCCGCAGGACA | 3665 | UGUCCUGCGGGGAAGGUGC |
| siRNA 1615 | 1615 | CACCUUCCCCGCAGGACAC | 3666 | GUGUCCUGCGGGGAAGGUG |
| siRNA 1616 | 1616 | ACCUUCCCCGCAGGACACA | 3667 | UGUGUCCUGCGGGGAAGGU |
| siRNA 1617 | 1617 | CCUUCCCCGCAGGACACAA | 3668 | UUGUGUCCUGCGGGGAAGG |
| siRNA 1618 | 1618 | CUUCCCCGCAGGACACAAG | 3669 | CUUGUGUCCUGCGGGGAAG |
| siRNA 1619 | 1619 | UUCCCCGCAGGACACAAGU | 3670 | ACUUGUGUCCUGCGGGGAA |
| siRNA 1620 | 1620 | UCCCCGCAGGACACAAGUG | 3671 | CACUUGUGUCCUGCGGGGA |
| siRNA 1621 | 1621 | CCCCGCAGGACACAAGUGC | 3672 | GCACUUGUGUCCUGCGGGG |
| siRNA 1622 | 1622 | CCCGCAGGACACAAGUGCC | 3673 | GGCACUUGUGUCCUGCGGG |
| siRNA 1623 | 1623 | CCGCAGGACACAAGUGCCA | 3674 | UGGCACUUGUGUCCUGCGG |
| SiRNA 1624 | 1624 | CGCAGGACACAAGUGCCAG | 3675 | CUGGCACUUGUGUCCUGCG |
| siRNA 1625 | 1625 | GCAGGACACAAGUGCCAGA | 3676 | UCUGGCACUUGUGUCCUGC |
| siRNA 1626 | 1626 | CAGGACACAAGUGCCAGAU | 3677 | AUCUGGCACUUGUGUCCUG |
| siRNA 1627 | 1627 | AGGACACAAGUGCCAGAUU | 3678 | AAUCUGGCACUUGUGUCCU |
| siRNA 1628 | 1628 | GGACACAAGUGCCAGAUUG | 3679 | CAAUCUGGCACUUGUGUCC |
| siRNA 1629 | 1629 | GACACAAGUGCCAGAUUGC | 3680 | GCAAUCUGGCACUUGUGUC |
| siRNA 1630 | 1630 | ACACAAGUGCCAGAUUGCG | 3681 | CGCAAUCUGGCACUUGUGU |
| siRNA 1631 | 1631 | CACAAGUGCCAGAUUGCGG | 3682 | CCGCAAUCUGGCACUUGUG |
| siRNA 1632 | 1632 | ACAAGUGCCAGAUUGCGGG | 3683 | CCCGCAAUCUGGCACUUGU |
| siRNA 1633 | 1633 | CAAGUGCCAGAUUGCGGGC | 3684 | GCCCGCAAUCUGGCACUUG |
| siRNA 1634 | 1634 | AAGUGCCAGAUUGCGGGCU | 3685 | AGCCCGCAAUCUGGCACUU |
| siRNA 1635 | 1635 | AGUGCCAGAUUGCGGGCUG | 3686 | CAGCCCGCAAUCUGGCACU |
| siRNA 1636 | 1636 | GUGCCAGAUUGCGGGCUGG | 3687 | CCAGCCCGCAAUCUGGCAC |
| siRNA 1637 | 1637 | UGCCAGAUUGCGGGCUGGG | 3688 | CCCAGCCCGCAAUCUGGCA |
| siRNA 1638 | 1638 | GCCAGAUUGCGGGCUGGGG | 3689 | CCCCAGCCCGCAAUCUGGC |
| siRNA 1639 | 1639 | CCAGAUUGCGGGCUGGGGC | 3690 | GCCCCAGCCCGCAAUCUGG |
| siRNA 1640 | 1640 | CAGAUUGCGGGCUGGGGCC | 3691 | GGCCCCAGCCCGCAAUCUG |
| siRNA 1641 | 1641 | AGAUUGCGGGCUGGGGCCA | 3692 | UGGCCCCAGCCCGCAAUCU |
| siRNA 1642 | 1642 | GAUUGCGGGCUGGGGCCAC | 3693 | GUGGCCCCAGCCCGCAAUC |
| siRNA 1643 | 1643 | AUUGCGGGCUGGGGCCACU | 3694 | AGUGGCCCCAGCCCGCAAU |
| siRNA 1644 | 1644 | UUGCGGGCUGGGGCCACUU | 3695 | AAGUGGCCCCAGCCCGCAA |
| siRNA 1645 | 1645 | UGCGGGCUGGGGCCACUUG | 3696 | CAAGUGGCCCCAGCCCGCA |
| siRNA 1646 | 1646 | GCGGGCUGGGGCCACUUGG | 3697 | CCAAGUGGCCCCAGCCCGC |
| siRNA 1647 | 1647 | CGGGCUGGGGCCACUUGGA | 3698 | UCCAAGUGGCCCCAGCCCG |
| siRNA 1648 | 1648 | GGGCUGGGGCCACUUGGAU | 3699 | AUCCAAGUGGCCCCAGCCC |
| SiRNA 1649 | 1649 | GGCUGGGGCCACUUGGAUG | 3700 | CAUCCAAGUGGCCCCAGCC |
| siRNA 1650 | 1650 | GCUGGGGCCACUUGGAUGA | 3701 | UCAUCCAAGUGGCCCCAGC |
| siRNA 1651 | 1651 | CUGGGGCCACUUGGAUGAG | 3702 | CUCAUCCAAGUGGCCCCAG |
| siRNA 1652 | 1652 | UGGGGCCACUUGGAUGAGA | 3703 | UCUCAUCCAAGUGGCCCCA |
| siRNA 1653 | 1653 | GGGGCCACUUGGAUGAGAA | 3704 | UUCUCAUCCAAGUGGCCCC |
| siRNA 1654 | 1654 | GGGCCACUUGGAUGAGAAC | 3705 | GUUCUCAUCCAAGUGGCCC |
| siRNA 1655 | 1655 | GGCCACUUGGAUGAGAACG | 3706 | CGUUCUCAUCCAAGUGGCC |
| siRNA 1656 | 1656 | GCCACUUGGAUGAGAACGU | 3707 | ACGUUCUCAUCCAAGUGGC |
| siRNA 1657 | 1657 | CCACUUGGAUGAGAACGUG | 3708 | CACGUUCUCAUCCAAGUGG |
| siRNA 1658 | 1658 | CACUUGGAUGAGAACGUGA | 3709 | UCACGUUCUCAUCCAAGUG |
| siRNA 1659 | 1659 | ACUUGGAUGAGAACGUGAG | 3710 | CUCACGUUCUCAUCCAAGU |
| siRNA 1660 | 1660 | CUUGGAUGAGAACGUGAGC | 3711 | GCUCACGUUCUCAUCCAAG |
| siRNA 1661 | 1661 | UUGGAUGAGAACGUGAGCG | 3712 | CGCUCACGUUCUCAUCCAA |
| siRNA 1662 | 1662 | UGGAUGAGAACGUGAGCGG | 3713 | CCGCUCACGUUCUCAUCCA |
| siRNA 1663 | 1663 | GGAUGAGAACGUGAGCGGC | 3714 | GCCGCUCACGUUCUCAUCC |
| siRNA 1664 | 1664 | GAUGAGAACGUGAGCGGCU | 3715 | AGCCGCUCACGUUCUCAUC |
| siRNA 1665 | 1665 | AUGAGAACGUGAGCGGCUA | 3716 | UAGCCGCUCACGUUCUCAU |
| siRNA 1666 | 1666 | UGAGAACGUGAGCGGCUAC | 3717 | GUAGCCGCUCACGUUCUCA |
| siRNA 1667 | 1667 | GAGAACGUGAGCGGCUACU | 3718 | AGUAGCCGCUCACGUUCUC |
| siRNA 1668 | 1668 | AGAACGUGAGCGGCUACUC | 3719 | GAGUAGCCGCUCACGUUCU |
| siRNA 1669 | 1669 | GAACGUGAGCGGCUACUCC | 3720 | GGAGUAGCCGCUCACGUUC |
| siRNA 1670 | 1670 | AACGUGAGCGGCUACUCCA | 3721 | UGGAGUAGCCGCUCACGUU |
| siRNA 1671 | 1671 | ACGUGAGCGGCUACUCCAG | 3722 | CUGGAGUAGCCGCUCACGU |
| siRNA 1672 | 1672 | CGUGAGCGGCUACUCCAGC | 3723 | GCUGGAGUAGCCGCUCACG |
| siRNA 1673 | 1673 | GUGAGCGGCUACUCCAGCU | 3724 | AGCUGGAGUAGCCGCUCAC |
| siRNA 1674 | 1674 | UGAGCGGCUACUCCAGCUC | 3725 | GAGCUGGAGUAGCCGCUCA |
| siRNA 1675 | 1675 | GAGCGGCUACUCCAGCUCC | 3726 | GGAGCUGGAGUAGCCGCUC |
| siRNA 1676 | 1676 | AGCGGCUACUCCAGCUCCC | 3727 | GGGAGCUGGAGUAGCCGCU |
| siRNA 1677 | 1677 | GCGGCUACUCCAGCUCCCU | 3728 | AGGGAGCUGGAGUAGCCGC |
| siRNA 1678 | 1678 | CGGCUACUCCAGCUCCCUG | 3729 | CAGGGAGCUGGAGUAGCCG |
| siRNA 1679 | 1679 | GGCUACUCCAGCUCCCUGC | 3730 | GCAGGGAGCUGGAGUAGCC |
| siRNA 1680 | 1680 | GCUACUCCAGCUCCCUGCG | 3731 | CGCAGGGAGCUGGAGUAGC |
| siRNA 1681 | 1681 | CUACUCCAGCUCCCUGCGG | 3732 | CCGCAGGGAGCUGGAGUAG |
| siRNA 1682 | 1682 | UACUCCAGCUCCCUGCGGG | 3733 | CCCGCAGGGAGCUGGAGUA |
| siRNA 1683 | 1683 | ACUCCAGCUCCCUGCGGGA | 3734 | UCCCGCAGGGAGCUGGAGU |
| siRNA 1684 | 1684 | CUCCAGCUCCCUGCGGGAG | 3735 | CUCCCGCAGGGAGCUGGAG |
| siRNA 1685 | 1685 | UCCAGCUCCCUGCGGGAGG | 3736 | CCUCCCGCAGGGAGCUGGA |
| siRNA 1686 | 1686 | CCAGCUCCCUGCGGGAGGC | 3737 | GCCUCCCGCAGGGAGCUGG |
| siRNA 1687 | 1687 | CAGCUCCCUGCGGGAGGCC | 3738 | GGCCUCCCGCAGGGAGCUG |
| siRNA 1688 | 1688 | AGCUCCCUGCGGGAGGCCC | 3739 | GGGCCUCCCGCAGGGAGCU |
| siRNA 1689 | 1689 | GCUCCCUGCGGGAGGCCCU | 3740 | AGGGCCUCCCGCAGGGAGC |
| siRNA 1690 | 1690 | CUCCCUGCGGGAGGCCCUG | 3741 | CAGGGCCUCCCGCAGGGAG |
| siRNA 1691 | 1691 | UCCCUGCGGGAGGCCCUGG | 3742 | CCAGGGCCUCCCGCAGGGA |
| siRNA 1692 | 1692 | CCCUGCGGGAGGCCCUGGU | 3743 | ACCAGGGCCUCCCGCAGGG |
| siRNA 1693 | 1693 | CCUGCGGGAGGCCCUGGUC | 3744 | GACCAGGGCCUCCCGCAGG |
| SiRNA 1694 | 1694 | CUGCGGGAGGCCCUGGUCC | 3745 | GGACCAGGGCCUCCCGCAG |
| siRNA 1695 | 1695 | UGCGGGAGGCCCUGGUCCC | 3746 | GGGACCAGGGCCUCCCGCA |
| siRNA 1696 | 1696 | GCGGGAGGCCCUGGUCCCC | 3747 | GGGGACCAGGGCCUCCCGC |
| siRNA 1697 | 1697 | CGGGAGGCCCUGGUCCCCC | 3748 | GGGGGACCAGGGCCUCCCG |
| siRNA 1698 | 1698 | GGGAGGCCCUGGUCCCCCU | 3749 | AGGGGGACCAGGGCCUCCC |
| siRNA 1699 | 1699 | GGAGGCCCUGGUCCCCCUG | 3750 | CAGGGGGACCAGGGCCUCC |
| siRNA 1700 | 1700 | GAGGCCCUGGUCCCCCUGG | 3751 | CCAGGGGGACCAGGGCCUC |
| siRNA 1701 | 1701 | AGGCCCUGGUCCCCCUGGU | 3752 | ACCAGGGGGACCAGGGCCU |
| siRNA 1702 | 1702 | GGCCCUGGUCCCCCUGGUC | 3753 | GACCAGGGGGACCAGGGCC |
| siRNA 1703 | 1703 | GCCCUGGUCCCCCUGGUCG | 3754 | CGACCAGGGGGACCAGGGC |
| siRNA 1704 | 1704 | CCCUGGUCCCCCUGGUCGC | 3755 | GCGACCAGGGGGACCAGGG |
| siRNA 1705 | 1705 | CCUGGUCCCCCUGGUCGCC | 3756 | GGCGACCAGGGGGACCAGG |
| siRNA 1706 | 1706 | CUGGUCCCCCUGGUCGCCG | 3757 | CGGCGACCAGGGGGACCAG |
| siRNA 1707 | 1707 | UGGUCCCCCUGGUCGCCGA | 3758 | UCGGCGACCAGGGGGACCA |
| siRNA 1708 | 1708 | GGUCCCCCUGGUCGCCGAC | 3759 | GUCGGCGACCAGGGGGACC |
| siRNA 1709 | 1709 | GUCCCCCUGGUCGCCGACC | 3760 | GGUCGGCGACCAGGGGGAC |
| siRNA 1710 | 1710 | UCCCCCUGGUCGCCGACCA | 3761 | UGGUCGGCGACCAGGGGGA |
| siRNA 1711 | 1711 | CCCCCUGGUCGCCGACCAC | 3762 | GUGGUCGGCGACCAGGGGG |
| siRNA 1712 | 1712 | CCCCUGGUCGCCGACCACA | 3763 | UGUGGUCGGCGACCAGGGG |
| siRNA 1713 | 1713 | CCCUGGUCGCCGACCACAA | 3764 | UUGUGGUCGGCGACCAGGG |
| siRNA 1714 | 1714 | CCUGGUCGCCGACCACAAG | 3765 | CUUGUGGUCGGCGACCAGG |
| siRNA 1715 | 1715 | CUGGUCGCCGACCACAAGU | 3766 | ACUUGUGGUCGGCGACCAG |
| siRNA 1716 | 1716 | UGGUCGCCGACCACAAGUG | 3767 | CACUUGUGGUCGGCGACCA |
| siRNA 1717 | 1717 | GGUCGCCGACCACAAGUGC | 3768 | GCACUUGUGGUCGGCGACC |
| siRNA 1718 | 1718 | GUCGCCGACCACAAGUGCA | 3769 | UGCACUUGUGGUCGGCGAC |
| siRNA 1719 | 1719 | UCGCCGACCACAAGUGCAG | 3770 | CUGCACUUGUGGUCGGCGA |
| siRNA 1720 | 1720 | CGCCGACCACAAGUGCAGC | 3771 | GCUGCACUUGUGGUCGGCG |
| siRNA 1721 | 1721 | GCCGACCACAAGUGCAGCA | 3772 | UGCUGCACUUGUGGUCGGC |
| siRNA 1722 | 1722 | CCGACCACAAGUGCAGCAG | 3773 | CUGCUGCACUUGUGGUCGG |
| siRNA 1723 | 1723 | CGACCACAAGUGCAGCAGC | 3774 | GCUGCUGCACUUGUGGUCG |
| siRNA 1724 | 1724 | GACCACAAGUGCAGCAGCC | 3775 | GGCUGCUGCACUUGUGGUC |
| siRNA 1725 | 1725 | ACCACAAGUGCAGCAGCCC | 3776 | GGGCUGCUGCACUUGUGGU |
| siRNA 1726 | 1726 | CCACAAGUGCAGCAGCCCU | 3777 | AGGGCUGCUGCACUUGUGG |
| siRNA 1727 | 1727 | CACAAGUGCAGCAGCCCUG | 3778 | CAGGGCUGCUGCACUUGUG |
| siRNA 1728 | 1728 | ACAAGUGCAGCAGCCCUGA | 3779 | UCAGGGCUGCUGCACUUGU |
| siRNA 1729 | 1729 | CAAGUGCAGCAGCCCUGAG | 3780 | CUCAGGGCUGCUGCACUUG |
| siRNA 1730 | 1730 | AAGUGCAGCAGCCCUGAGG | 3781 | CCUCAGGGCUGCUGCACUU |
| siRNA 1731 | 1731 | AGUGCAGCAGCCCUGAGGU | 3782 | ACCUCAGGGCUGCUGCACU |
| siRNA 1732 | 1732 | GUGCAGCAGCCCUGAGGUC | 3783 | GACCUCAGGGCUGCUGCAC |
| siRNA 1733 | 1733 | UGCAGCAGCCCUGAGGUCU | 3784 | AGACCUCAGGGCUGCUGCA |
| siRNA 1734 | 1734 | GCAGCAGCCCUGAGGUCUA | 3785 | UAGACCUCAGGGCUGCUGC |
| siRNA 1735 | 1735 | CAGCAGCCCUGAGGUCUAC | 3786 | GUAGACCUCAGGGCUGCUG |
| siRNA 1736 | 1736 | AGCAGCCCUGAGGUCUACG | 3787 | CGUAGACCUCAGGGCUGCU |
| siRNA 1737 | 1737 | GCAGCCCUGAGGUCUACGG | 3788 | CCGUAGACCUCAGGGCUGC |
| siRNA 1738 | 1738 | CAGCCCUGAGGUCUACGGC | 3789 | GCCGUAGACCUCAGGGCUG |
| siRNA 1739 | 1739 | AGCCCUGAGGUCUACGGCG | 3790 | CGCCGUAGACCUCAGGGCU |
| siRNA 1740 | 1740 | GCCCUGAGGUCUACGGCGC | 3791 | GCGCCGUAGACCUCAGGGC |
| siRNA 1741 | 1741 | CCCUGAGGUCUACGGCGCC | 3792 | GGCGCCGUAGACCUCAGGG |
| siRNA 1742 | 1742 | CCUGAGGUCUACGGCGCCG | 3793 | CGGCGCCGUAGACCUCAGG |
| siRNA 1743 | 1743 | CUGAGGUCUACGGCGCCGA | 3794 | UCGGCGCCGUAGACCUCAG |
| siRNA 1744 | 1744 | UGAGGUCUACGGCGCCGAC | 3795 | GUCGGCGCCGUAGACCUCA |
| siRNA 1745 | 1745 | GAGGUCUACGGCGCCGACA | 3796 | UGUCGGCGCCGUAGACCUC |
| siRNA 1746 | 1746 | AGGUCUACGGCGCCGACAU | 3797 | AUGUCGGCGCCGUAGACCU |
| siRNA 1747 | 1747 | GGUCUACGGCGCCGACAUC | 3798 | GAUGUCGGCGCCGUAGACC |
| siRNA 1748 | 1748 | GUCUACGGCGCCGACAUCA | 3799 | UGAUGUCGGCGCCGUAGAC |
| siRNA 1749 | 1749 | UCUACGGCGCCGACAUCAG | 3800 | CUGAUGUCGGCGCCGUAGA |
| siRNA 1750 | 1750 | CUACGGCGCCGACAUCAGC | 3801 | GCUGAUGUCGGCGCCGUAG |
| siRNA 1751 | 1751 | UACGGCGCCGACAUCAGCC | 3802 | GGCUGAUGUCGGCGCCGUA |
| siRNA 1752 | 1752 | ACGGCGCCGACAUCAGCCC | 3803 | GGGCUGAUGUCGGCGCCGU |
| siRNA 1753 | 1753 | CGGCGCCGACAUCAGCCCC | 3804 | GGGGCUGAUGUCGGCGCCG |
| siRNA 1754 | 1754 | GGCGCCGACAUCAGCCCCA | 3805 | UGGGGCUGAUGUCGGCGCC |
| siRNA 1755 | 1755 | GCGCCGACAUCAGCCCCAA | 3806 | UUGGGGCUGAUGUCGGCGC |
| siRNA 1756 | 1756 | CGCCGACAUCAGCCCCAAC | 3807 | GUUGGGGCUGAUGUCGGCG |
| siRNA 1757 | 1757 | GCCGACAUCAGCCCCAACA | 3808 | UGUUGGGGCUGAUGUCGGC |
| siRNA 1758 | 1758 | CCGACAUCAGCCCCAACAU | 3809 | AUGUUGGGGCUGAUGUCGG |
| siRNA 1759 | 1759 | CGACAUCAGCCCCAACAUG | 3810 | CAUGUUGGGGCUGAUGUCG |
| siRNA 1760 | 1760 | GACAUCAGCCCCAACAUGC | 3811 | GCAUGUUGGGGCUGAUGUC |
| siRNA 1761 | 1761 | ACAUCAGCCCCAACAUGCU | 3812 | AGCAUGUUGGGGCUGAUGU |
| siRNA 1762 | 1762 | CAUCAGCCCCAACAUGCUC | 3813 | GAGCAUGUUGGGGCUGAUG |
| siRNA 1763 | 1763 | AUCAGCCCCAACAUGCUCU | 3814 | AGAGCAUGUUGGGGCUGAU |
| siRNA 1764 | 1764 | UCAGCCCCAACAUGCUCUG | 3815 | CAGAGCAUGUUGGGGCUGA |
| siRNA 1765 | 1765 | CAGCCCCAACAUGCUCUGU | 3816 | ACAGAGCAUGUUGGGGCUG |
| siRNA 1766 | 1766 | AGCCCCAACAUGCUCUGUG | 3817 | CACAGAGCAUGUUGGGGCU |
| siRNA 1767 | 1767 | GCCCCAACAUGCUCUGUGC | 3818 | GCACAGAGCAUGUUGGGGC |
| siRNA 1768 | 1768 | CCCCAACAUGCUCUGUGCC | 3819 | GGCACAGAGCAUGUUGGGG |
| siRNA 1769 | 1769 | CCCAACAUGCUCUGUGCCG | 3820 | CGGCACAGAGCAUGUUGGG |
| siRNA 1770 | 1770 | CCAACAUGCUCUGUGCCGG | 3821 | CCGGCACAGAGCAUGUUGG |
| siRNA 1771 | 1771 | CAACAUGCUCUGUGCCGGC | 3822 | GCCGGCACAGAGCAUGUUG |
| siRNA 1772 | 1772 | AACAUGCUCUGUGCCGGCU | 3823 | AGCCGGCACAGAGCAUGUU |
| siRNA 1773 | 1773 | ACAUGCUCUGUGCCGGCUA | 3824 | UAGCCGGCACAGAGCAUGU |
| siRNA 1774 | 1774 | CAUGCUCUGUGCCGGCUAC | 3825 | GUAGCCGGCACAGAGCAUG |
| sIRNA 1775 | 1775 | AUGCUCUGUGCCGGCUACU | 3826 | AGUAGCCGGCACAGAGCAU |
| siRNA 1776 | 1776 | UGCUCUGUGCCGGCUACUU | 3827 | AAGUAGCCGGCACAGAGCA |
| siRNA 1777 | 1777 | GCUCUGUGCCGGCUACUUC | 3828 | GAAGUAGCCGGCACAGAGC |
| siRNA 1778 | 1778 | CUCUGUGCCGGCUACUUCG | 3829 | CGAAGUAGCCGGCACAGAG |
| siRNA 1779 | 1779 | UCUGUGCCGGCUACUUCGA | 3830 | UCGAAGUAGCCGGCACAGA |
| siRNA 1780 | 1780 | CUGUGCCGGCUACUUCGAC | 3831 | GUCGAAGUAGCCGGCACAG |
| siRNA 1781 | 1781 | UGUGCCGGCUACUUCGACU | 3832 | AGUCGAAGUAGCCGGCACA |
| siRNA 1782 | 1782 | GUGCCGGCUACUUCGACUG | 3833 | CAGUCGAAGUAGCCGGCAC |
| siRNA 1783 | 1783 | UGCCGGCUACUUCGACUGC | 3834 | GCAGUCGAAGUAGCCGGCA |
| siRNA 1784 | 1784 | GCCGGCUACUUCGACUGCA | 3835 | UGCAGUCGAAGUAGCCGGC |
| siRNA 1785 | 1785 | CCGGCUACUUCGACUGCAA | 3836 | UUGCAGUCGAAGUAGCCGG |
| siRNA 1786 | 1786 | CGGCUACUUCGACUGCAAG | 3837 | CUUGCAGUCGAAGUAGCCG |
| siRNA 1787 | 1787 | GGCUACUUCGACUGCAAGU | 3838 | ACUUGCAGUCGAAGUAGCC |
| siRNA 1788 | 1788 | GCUACUUCGACUGCAAGUC | 3839 | GACUUGCAGUCGAAGUAGC |
| siRNA 1789 | 1789 | CUACUUCGACUGCAAGUCC | 3840 | GGACUUGCAGUCGAAGUAG |
| siRNA 1790 | 1790 | UACUUCGACUGCAAGUCCG | 3841 | CGGACUUGCAGUCGAAGUA |
| siRNA 1791 | 1791 | ACUUCGACUGCAAGUCCGA | 3842 | UCGGACUUGCAGUCGAAGU |
| siRNA 1792 | 1792 | CUUCGACUGCAAGUCCGAC | 3843 | GUCGGACUUGCAGUCGAAG |
| siRNA 1793 | 1793 | UUCGACUGCAAGUCCGACG | 3844 | CGUCGGACUUGCAGUCGAA |
| siRNA 1794 | 1794 | UCGACUGCAAGUCCGACGC | 3845 | GCGUCGGACUUGCAGUCGA |
| siRNA 1795 | 1795 | CGACUGCAAGUCCGACGCC | 3846 | GGCGUCGGACUUGCAGUCG |
| siRNA 1796 | 1796 | GACUGCAAGUCCGACGCCU | 3847 | AGGCGUCGGACUUGCAGUC |
| siRNA 1797 | 1797 | ACUGCAAGUCCGACGCCUG | 3848 | CAGGCGUCGGACUUGCAGU |
| siRNA 1798 | 1798 | CUGCAAGUCCGACGCCUGC | 3849 | GCAGGCGUCGGACUUGCAG |
| SiRNA 1799 | 1799 | UGCAAGUCCGACGCCUGCC | 3850 | GGCAGGCGUCGGACUUGCA |
| siRNA 1800 | 1800 | GCAAGUCCGACGCCUGCCA | 3851 | UGGCAGGCGUCGGACUUGC |
| siRNA 1801 | 1801 | CAAGUCCGACGCCUGCCAG | 3852 | CUGGCAGGCGUCGGACUUG |
| siRNA 1802 | 1802 | AAGUCCGACGCCUGCCAGG | 3853 | CCUGGCAGGCGUCGGACUU |
| siRNA 1803 | 1803 | AGUCCGACGCCUGCCAGGG | 3854 | CCCUGGCAGGCGUCGGACU |
| siRNA 1804 | 1804 | GUCCGACGCCUGCCAGGGG | 3855 | CCCCUGGCAGGCGUCGGAC |
| siRNA 1805 | 1805 | UCCGACGCCUGCCAGGGGG | 3856 | CCCCCUGGCAGGCGUCGGA |
| siRNA 1806 | 1806 | CCGACGCCUGCCAGGGGGA | 3857 | UCCCCCUGGCAGGCGUCGG |
| siRNA 1807 | 1807 | CGACGCCUGCCAGGGGGAC | 3858 | GUCCCCCUGGCAGGCGUCG |
| siRNA 1808 | 1808 | GACGCCUGCCAGGGGGACU | 3859 | AGUCCCCCUGGCAGGCGUC |
| siRNA 1809 | 1809 | ACGCCUGCCAGGGGGACUC | 3860 | GAGUCCCCCUGGCAGGCGU |
| siRNA 1810 | 1810 | CGCCUGCCAGGGGGACUCA | 3861 | UGAGUCCCCCUGGCAGGCG |
| siRNA 1811 | 1811 | GCCUGCCAGGGGGACUCAG | 3862 | CUGAGUCCCCCUGGCAGGC |
| siRNA 1812 | 1812 | CCUGCCAGGGGGACUCAGG | 3863 | CCUGAGUCCCCCUGGCAGG |
| siRNA 1813 | 1813 | CUGCCAGGGGGACUCAGGG | 3864 | CCCUGAGUCCCCCUGGCAG |
| siRNA 1814 | 1814 | UGCCAGGGGGACUCAGGGG | 3865 | CCCCUGAGUCCCCCUGGCA |
| siRNA 1815 | 1815 | GCCAGGGGGACUCAGGGGG | 3866 | CCCCCUGAGUCCCCCUGGC |
| siRNA 1816 | 1816 | CCAGGGGGACUCAGGGGGG | 3867 | CCCCCCUGAGUCCCCCUGG |
| siRNA 1817 | 1817 | CAGGGGGACUCAGGGGGGC | 3868 | GCCCCCCUGAGUCCCCCUG |
| siRNA 1818 | 1818 | AGGGGGACUCAGGGGGGCC | 3869 | GGCCCCCCUGAGUCCCCCU |
| siRNA 1819 | 1819 | GGGGGACUCAGGGGGGCCC | 3870 | GGGCCCCCCUGAGUCCCCC |
| siRNA 1820 | 1820 | GGGGACUCAGGGGGGCCCC | 3871 | GGGGCCCCCCUGAGUCCCC |
| siRNA 1821 | 1821 | GGGACUCAGGGGGGCCCCU | 3872 | AGGGGCCCCCCUGAGUCCC |
| siRNA 1822 | 1822 | GGACUCAGGGGGGCCCCUG | 3873 | CAGGGGCCCCCCUGAGUCC |
| siRNA 1823 | 1823 | GACUCAGGGGGGCCCCUGG | 3874 | CCAGGGGCCCCCCUGAGUC |
| siRNA 1824 | 1824 | ACUCAGGGGGGCCCCUGGC | 3875 | GCCAGGGGCCCCCCUGAGU |
| siRNA 1825 | 1825 | CUCAGGGGGGCCCCUGGCC | 3876 | GGCCAGGGGCCCCCCUGAG |
| siRNA 1826 | 1826 | UCAGGGGGGCCCCUGGCCU | 3877 | AGGCCAGGGGCCCCCCUGA |
| siRNA 1827 | 1827 | CAGGGGGGCCCCUGGCCUG | 3878 | CAGGCCAGGGGCCCCCCUG |
| siRNA 1828 | 1828 | AGGGGGGCCCCUGGCCUGC | 3879 | GCAGGCCAGGGGCCCCCCU |
| siRNA 1829 | 1829 | GGGGGGCCCCUGGCCUGCG | 3880 | CGCAGGCCAGGGGCCCCCC |
| siRNA 1830 | 1830 | GGGGGCCCCUGGCCUGCGA | 3881 | UCGCAGGCCAGGGGCCCCC |
| siRNA 1831 | 1831 | GGGGCCCCUGGCCUGCGAG | 3882 | CUCGCAGGCCAGGGGCCCC |
| siRNA 1832 | 1832 | GGGCCCCUGGCCUGCGAGA | 3883 | UCUCGCAGGCCAGGGGCCC |
| siRNA 1833 | 1833 | GGCCCCUGGCCUGCGAGAA | 3884 | UUCUCGCAGGCCAGGGGCC |
| siRNA 1834 | 1834 | GCCCCUGGCCUGCGAGAAG | 3885 | CUUCUCGCAGGCCAGGGGC |
| siRNA 1835 | 1835 | CCCCUGGCCUGCGAGAAGA | 3886 | UCUUCUCGCAGGCCAGGGG |
| siRNA 1836 | 1836 | CCCUGGCCUGCGAGAAGAA | 3887 | UUCUUCUCGCAGGCCAGGG |
| siRNA 1837 | 1837 | CCUGGCCUGCGAGAAGAAC | 3888 | GUUCUUCUCGCAGGCCAGG |
| siRNA 1838 | 1838 | CUGGCCUGCGAGAAGAACG | 3889 | CGUUCUUCUCGCAGGCCAG |
| siRNA 1839 | 1839 | UGGCCUGCGAGAAGAACGG | 3890 | CCGUUCUUCUCGCAGGCCA |
| siRNA 1840 | 1840 | GGCCUGCGAGAAGAACGGC | 3891 | GCCGUUCUUCUCGCAGGCC |
| siRNA 1841 | 1841 | GCCUGCGAGAAGAACGGCG | 3892 | CGCCGUUCUUCUCGCAGGC |
| siRNA 1842 | 1842 | CCUGCGAGAAGAACGGCGU | 3893 | ACGCCGUUCUUCUCGCAGG |
| siRNA 1843 | 1843 | CUGCGAGAAGAACGGCGUG | 3894 | CACGCCGUUCUUCUCGCAG |
| siRNA 1844 | 1844 | UGCGAGAAGAACGGCGUGG | 3895 | CCACGCCGUUCUUCUCGCA |
| siRNA 1845 | 1845 | GCGAGAAGAACGGCGUGGC | 3896 | GCCACGCCGUUCUUCUCGC |
| siRNA 1846 | 1846 | CGAGAAGAACGGCGUGGCU | 3897 | AGCCACGCCGUUCUUCUCG |
| siRNA 1847 | 1847 | GAGAAGAACGGCGUGGCUU | 3898 | AAGCCACGCCGUUCUUCUC |
| siRNA 1848 | 1848 | AGAAGAACGGCGUGGCUUA | 3899 | UAAGCCACGCCGUUCUUCU |
| siRNA 1849 | 1849 | GAAGAACGGCGUGGCUUAC | 3900 | GUAAGCCACGCCGUUCUUC |
| siRNA 1850 | 1850 | AAGAACGGCGUGGCUUACC | 3901 | GGUAAGCCACGCCGUUCUU |
| siRNA 1851 | 1851 | AGAACGGCGUGGCUUACCU | 3902 | AGGUAAGCCACGCCGUUCU |
| siRNA 1852 | 1852 | GAACGGCGUGGCUUACCUC | 3903 | GAGGUAAGCCACGCCGUUC |
| siRNA 1853 | 1853 | AACGGCGUGGCUUACCUCU | 3904 | AGAGGUAAGCCACGCCGUU |
| siRNA 1854 | 1854 | ACGGCGUGGCUUACCUCUA | 3905 | UAGAGGUAAGCCACGCCGU |
| siRNA 1855 | 1855 | CGGCGUGGCUUACCUCUAC | 3906 | GUAGAGGUAAGCCACGCCG |
| siRNA 1856 | 1856 | GGCGUGGCUUACCUCUACG | 3907 | CGUAGAGGUAAGCCACGCC |
| siRNA 1857 | 1857 | GCGUGGCUUACCUCUACGG | 3908 | CCGUAGAGGUAAGCCACGC |
| siRNA 1858 | 1858 | CGUGGCUUACCUCUACGGC | 3909 | GCCGUAGAGGUAAGCCACG |
| siRNA 1859 | 1859 | GUGGCUUACCUCUACGGCA | 3910 | UGCCGUAGAGGUAAGCCAC |
| siRNA 1860 | 1860 | UGGCUUACCUCUACGGCAU | 3911 | AUGCCGUAGAGGUAAGCCA |
| siRNA 1861 | 1861 | GGCUUACCUCUACGGCAUC | 3912 | GAUGCCGUAGAGGUAAGCC |
| siRNA 1862 | 1862 | GCUUACCUCUACGGCAUCA | 3913 | UGAUGCCGUAGAGGUAAGC |
| siRNA 1863 | 1863 | CUUACCUCUACGGCAUCAU | 3914 | AUGAUGCCGUAGAGGUAAG |
| siRNA 1864 | 1864 | UUACCUCUACGGCAUCAUC | 3915 | GAUGAUGCCGUAGAGGUAA |
| siRNA 1865 | 1865 | UACCUCUACGGCAUCAUCA | 3916 | UGAUGAUGCCGUAGAGGUA |
| siRNA 1866 | 1866 | ACCUCUACGGCAUCAUCAG | 3917 | CUGAUGAUGCCGUAGAGGU |
| siRNA 1867 | 1867 | CCUCUACGGCAUCAUCAGC | 3918 | GCUGAUGAUGCCGUAGAGG |
| SiRNA 1868 | 1868 | CUCUACGGCAUCAUCAGCU | 3919 | AGCUGAUGAUGCCGUAGAG |
| siRNA 1869 | 1869 | UCUACGGCAUCAUCAGCUG | 3920 | CAGCUGAUGAUGCCGUAGA |
| siRNA 1870 | 1870 | CUACGGCAUCAUCAGCUGG | 3921 | CCAGCUGAUGAUGCCGUAG |
| siRNA 1871 | 1871 | UACGGCAUCAUCAGCUGGG | 3922 | CCCAGCUGAUGAUGCCGUA |
| siRNA 1872 | 1872 | ACGGCAUCAUCAGCUGGGG | 3923 | CCCCAGCUGAUGAUGCCGU |
| siRNA 1873 | 1873 | CGGCAUCAUCAGCUGGGGU | 3924 | ACCCCAGCUGAUGAUGCCG |
| siRNA 1874 | 1874 | GGCAUCAUCAGCUGGGGUG | 3925 | CACCCCAGCUGAUGAUGCC |
| siRNA 1875 | 1875 | GCAUCAUCAGCUGGGGUGA | 3926 | UCACCCCAGCUGAUGAUGC |
| siRNA 1876 | 1876 | CAUCAUCAGCUGGGGUGAC | 3927 | GUCACCCCAGCUGAUGAUG |
| siRNA 1877 | 1877 | AUCAUCAGCUGGGGUGACG | 3928 | CGUCACCCCAGCUGAUGAU |
| siRNA 1878 | 1878 | UCAUCAGCUGGGGUGACGG | 3929 | CCGUCACCCCAGCUGAUGA |
| siRNA 1879 | 1879 | CAUCAGCUGGGGUGACGGC | 3930 | GCCGUCACCCCAGCUGAUG |
| siRNA 1880 | 1880 | AUCAGCUGGGGUGACGGCU | 3931 | AGCCGUCACCCCAGCUGAU |
| siRNA 1881 | 1881 | UCAGCUGGGGUGACGGCUG | 3932 | CAGCCGUCACCCCAGCUGA |
| siRNA 1882 | 1882 | CAGCUGGGGUGACGGCUGC | 3933 | GCAGCCGUCACCCCAGCUG |
| siRNA 1883 | 1883 | AGCUGGGGUGACGGCUGCG | 3934 | CGCAGCCGUCACCCCAGCU |
| siRNA 1884 | 1884 | GCUGGGGUGACGGCUGCGG | 3935 | CCGCAGCCGUCACCCCAGC |
| siRNA 1885 | 1885 | CUGGGGUGACGGCUGCGGG | 3936 | CCCGCAGCCGUCACCCCAG |
| siRNA 1886 | 1886 | UGGGGUGACGGCUGCGGGC | 3937 | GCCCGCAGCCGUCACCCCA |
| siRNA 1887 | 1887 | GGGGUGACGGCUGCGGGCG | 3938 | CGCCCGCAGCCGUCACCCC |
| siRNA 1888 | 1888 | GGGUGACGGCUGCGGGCGG | 3939 | CCGCCCGCAGCCGUCACCC |
| siRNA 1889 | 1889 | GGUGACGGCUGCGGGCGGC | 3940 | GCCGCCCGCAGCCGUCACC |
| siRNA 1890 | 1890 | GUGACGGCUGCGGGCGGCU | 3941 | AGCCGCCCGCAGCCGUCAC |
| siRNA 1891 | 1891 | UGACGGCUGCGGGCGGCUC | 3942 | GAGCCGCCCGCAGCCGUCA |
| siRNA 1892 | 1892 | GACGGCUGCGGGCGGCUCC | 3943 | GGAGCCGCCCGCAGCCGUC |
| siRNA 1893 | 1893 | ACGGCUGCGGGCGGCUCCA | 3944 | UGGAGCCGCCCGCAGCCGU |
| siRNA 1894 | 1894 | CGGCUGCGGGCGGCUCCAC | 3945 | GUGGAGCCGCCCGCAGCCG |
| siRNA 1895 | 1895 | GGCUGCGGGCGGCUCCACA | 3946 | UGUGGAGCCGCCCGCAGCC |
| siRNA 1896 | 1896 | GCUGCGGGCGGCUCCACAA | 3947 | UUGUGGAGCCGCCCGCAGC |
| siRNA 1897 | 1897 | CUGCGGGCGGCUCCACAAG | 3948 | CUUGUGGAGCCGCCCGCAG |
| siRNA 1898 | 1898 | UGCGGGCGGCUCCACAAGC | 3949 | GCUUGUGGAGCCGCCCGCA |
| siRNA 1899 | 1899 | GCGGGCGGCUCCACAAGCC | 3950 | GGCUUGUGGAGCCGCCCGC |
| siRNA 1900 | 1900 | CGGGCGGCUCCACAAGCCG | 3951 | CGGCUUGUGGAGCCGCCCG |
| siRNA 1901 | 1901 | GGGCGGCUCCACAAGCCGG | 3952 | CCGGCUUGUGGAGCCGCCC |
| SiRNA 1902 | 1902 | GGCGGCUCCACAAGCCGGG | 3953 | CCCGGCUUGUGGAGCCGCC |
| siRNA 1903 | 1903 | GCGGCUCCACAAGCCGGGG | 3954 | CCCCGGCUUGUGGAGCCGC |
| siRNA 1904 | 1904 | CGGCUCCACAAGCCGGGGG | 3955 | CCCCCGGCUUGUGGAGCCG |
| siRNA 1905 | 1905 | GGCUCCACAAGCCGGGGGU | 3956 | ACCCCCGGCUUGUGGAGCC |
| siRNA 1906 | 1906 | GCUCCACAAGCCGGGGGUC | 3957 | GACCCCCGGCUUGUGGAGC |
| siRNA 1907 | 1907 | CUCCACAAGCCGGGGGUCU | 3958 | AGACCCCCGGCUUGUGGAG |
| siRNA 1908 | 1908 | UCCACAAGCCGGGGGUCUA | 3959 | UAGACCCCCGGCUUGUGGA |
| siRNA 1909 | 1909 | CCACAAGCCGGGGGUCUAC | 3960 | GUAGACCCCCGGCUUGUGG |
| siRNA 1910 | 1910 | CACAAGCCGGGGGUCUACA | 3961 | UGUAGACCCCCGGCUUGUG |
| siRNA 1911 | 1911 | ACAAGCCGGGGGUCUACAC | 3962 | GUGUAGACCCCCGGCUUGU |
| siRNA 1912 | 1912 | CAAGCCGGGGGUCUACACC | 3963 | GGUGUAGACCCCCGGCUUG |
| siRNA 1913 | 1913 | AAGCCGGGGGUCUACACCC | 3964 | GGGUGUAGACCCCCGGCUU |
| siRNA 1914 | 1914 | AGCCGGGGGUCUACACCCG | 3965 | CGGGUGUAGACCCCCGGCU |
| siRNA 1915 | 1915 | GCCGGGGGUCUACACCCGC | 3966 | GCGGGUGUAGACCCCCGGC |
| siRNA 1916 | 1916 | CCGGGGGUCUACACCCGCG | 3967 | CGCGGGUGUAGACCCCCGG |
| siRNA 1917 | 1917 | CGGGGGUCUACACCCGCGU | 3968 | ACGCGGGUGUAGACCCCCG |
| siRNA 1918 | 1918 | GGGGGUCUACACCCGCGUG | 3969 | CACGCGGGUGUAGACCCCC |
| siRNA 1919 | 1919 | GGGGUCUACACCCGCGUGG | 3970 | CCACGCGGGUGUAGACCCC |
| siRNA 1920 | 1920 | GGGUCUACACCCGCGUGGC | 3971 | GCCACGCGGGUGUAGACCC |
| siRNA 1921 | 1921 | GGUCUACACCCGCGUGGCC | 3972 | GGCCACGCGGGUGUAGACC |
| siRNA 1922 | 1922 | GUCUACACCCGCGUGGCCA | 3973 | UGGCCACGCGGGUGUAGAC |
| siRNA 1923 | 1923 | UCUACACCCGCGUGGCCAA | 3974 | UUGGCCACGCGGGUGUAGA |
| siRNA 1924 | 1924 | CUACACCCGCGUGGCCAAC | 3975 | GUUGGCCACGCGGGUGUAG |
| siRNA 1925 | 1925 | UACACCCGCGUGGCCAACU | 3976 | AGUUGGCCACGCGGGUGUA |
| siRNA 1926 | 1926 | ACACCCGCGUGGCCAACUA | 3977 | UAGUUGGCCACGCGGGUGU |
| siRNA 1927 | 1927 | CACCCGCGUGGCCAACUAU | 3978 | AUAGUUGGCCACGCGGGUG |
| siRNA 1928 | 1928 | ACCCGCGUGGCCAACUAUG | 3979 | CAUAGUUGGCCACGCGGGU |
| siRNA 1929 | 1929 | CCCGCGUGGCCAACUAUGU | 3980 | ACAUAGUUGGCCACGCGGG |
| siRNA 1930 | 1930 | CCGCGUGGCCAACUAUGUG | 3981 | CACAUAGUUGGCCACGCGG |
| siRNA 1931 | 1931 | CGCGUGGCCAACUAUGUGG | 3982 | CCACAUAGUUGGCCACGCG |
| siRNA 1932 | 1932 | GCGUGGCCAACUAUGUGGA | 3983 | UCCACAUAGUUGGCCACGC |
| siRNA 1933 | 1933 | CGUGGCCAACUAUGUGGAC | 3984 | GUCCACAUAGUUGGCCACG |
| siRNA 1934 | 1934 | GUGGCCAACUAUGUGGACU | 3985 | AGUCCACAUAGUUGGCCAC |
| siRNA 1935 | 1935 | UGGCCAACUAUGUGGACUG | 3986 | CAGUCCACAUAGUUGGCCA |
| siRNA 1936 | 1936 | GGCCAACUAUGUGGACUGG | 3987 | CCAGUCCACAUAGUUGGCC |
| siRNA 1937 | 1937 | GCCAACUAUGUGGACUGGA | 3988 | UCCAGUCCACAUAGUUGGC |
| siRNA 1938 | 1938 | CCAACUAUGUGGACUGGAU | 3989 | AUCCAGUCCACAUAGUUGG |
| siRNA 1939 | 1939 | CAACUAUGUGGACUGGAUC | 3990 | GAUCCAGUCCACAUAGUUG |
| SiRNA 1940 | 1940 | AACUAUGUGGACUGGAUCA | 3991 | UGAUCCAGUCCACAUAGUU |
| siRNA 1941 | 1941 | ACUAUGUGGACUGGAUCAA | 3992 | UUGAUCCAGUCCACAUAGU |
| siRNA 1942 | 1942 | CUAUGUGGACUGGAUCAAC | 3993 | GUUGAUCCAGUCCACAUAG |
| siRNA 1943 | 1943 | UAUGUGGACUGGAUCAACG | 3994 | CGUUGAUCCAGUCCACAUA |
| siRNA 1944 | 1944 | AUGUGGACUGGAUCAACGA | 3995 | UCGUUGAUCCAGUCCACAU |
| siRNA 1945 | 1945 | UGUGGACUGGAUCAACGAC | 3996 | GUCGUUGAUCCAGUCCACA |
| siRNA 1946 | 1946 | GUGGACUGGAUCAACGACC | 3997 | GGUCGUUGAUCCAGUCCAC |
| siRNA 1947 | 1947 | UGGACUGGAUCAACGACCG | 3998 | CGGUCGUUGAUCCAGUCCA |
| siRNA 1948 | 1948 | GGACUGGAUCAACGACCGG | 3999 | CCGGUCGUUGAUCCAGUCC |
| siRNA 1949 | 1949 | GACUGGAUCAACGACCGGA | 4000 | UCCGGUCGUUGAUCCAGUC |
| siRNA 1950 | 1950 | ACUGGAUCAACGACCGGAU | 4001 | AUCCGGUCGUUGAUCCAGU |
| siRNA 1951 | 1951 | CUGGAUCAACGACCGGAUA | 4002 | UAUCCGGUCGUUGAUCCAG |
| siRNA 1952 | 1952 | UGGAUCAACGACCGGAUAC | 4003 | GUAUCCGGUCGUUGAUCCA |
| siRNA 1953 | 1953 | GGAUCAACGACCGGAUACG | 4004 | CGUAUCCGGUCGUUGAUCC |
| SiRNA 1954 | 1954 | GAUCAACGACCGGAUACGG | 4005 | CCGUAUCCGGUCGUUGAUC |
| siRNA 1955 | 1955 | AUCAACGACCGGAUACGGC | 4006 | GCCGUAUCCGGUCGUUGAU |
| siRNA 1956 | 1956 | UCAACGACCGGAUACGGCC | 4007 | GGCCGUAUCCGGUCGUUGA |
| siRNA 1957 | 1957 | CAACGACCGGAUACGGCCU | 4008 | AGGCCGUAUCCGGUCGUUG |
| siRNA 1958 | 1958 | AACGACCGGAUACGGCCUC | 4009 | GAGGCCGUAUCCGGUCGUU |
| siRNA 1959 | 1959 | ACGACCGGAUACGGCCUCC | 4010 | GGAGGCCGUAUCCGGUCGU |
| siRNA 1960 | 1960 | CGACCGGAUACGGCCUCCC | 4011 | GGGAGGCCGUAUCCGGUCG |
| siRNA 1961 | 1961 | GACCGGAUACGGCCUCCCA | 4012 | UGGGAGGCCGUAUCCGGUC |
| siRNA 1962 | 1962 | ACCGGAUACGGCCUCCCAG | 4013 | CUGGGAGGCCGUAUCCGGU |
| siRNA 1963 | 1963 | CCGGAUACGGCCUCCCAGG | 4014 | CCUGGGAGGCCGUAUCCGG |
| siRNA 1964 | 1964 | CGGAUACGGCCUCCCAGGC | 4015 | GCCUGGGAGGCCGUAUCCG |
| siRNA 1965 | 1965 | GGAUACGGCCUCCCAGGCG | 4016 | CGCCUGGGAGGCCGUAUCC |
| siRNA 1966 | 1966 | GAUACGGCCUCCCAGGCGG | 4017 | CCGCCUGGGAGGCCGUAUC |
| siRNA 1967 | 1967 | AUACGGCCUCCCAGGCGGC | 4018 | GCCGCCUGGGAGGCCGUAU |
| siRNA 1968 | 1968 | UACGGCCUCCCAGGCGGCU | 4019 | AGCCGCCUGGGAGGCCGUA |
| siRNA 1969 | 1969 | ACGGCCUCCCAGGCGGCUU | 4020 | AAGCCGCCUGGGAGGCCGU |
| siRNA 1970 | 1970 | CGGCCUCCCAGGCGGCUUG | 4021 | CAAGCCGCCUGGGAGGCCG |
| siRNA 1971 | 1971 | GGCCUCCCAGGCGGCUUGU | 4022 | ACAAGCCGCCUGGGAGGCC |
| siRNA 1972 | 1972 | GCCUCCCAGGCGGCUUGUG | 4023 | CACAAGCCGCCUGGGAGGC |
| siRNA 1973 | 1973 | CCUCCCAGGCGGCUUGUGG | 4024 | CCACAAGCCGCCUGGGAGG |
| siRNA 1974 | 1974 | CUCCCAGGCGGCUUGUGGC | 4025 | GCCACAAGCCGCCUGGGAG |
| siRNA 1975 | 1975 | UCCCAGGCGGCUUGUGGCU | 4026 | AGCCACAAGCCGCCUGGGA |
| siRNA 1976 | 1976 | CCCAGGCGGCUUGUGGCUC | 4027 | GAGCCACAAGCCGCCUGGG |
| siRNA 1977 | 1977 | CCAGGCGGCUUGUGGCUCC | 4028 | GGAGCCACAAGCCGCCUGG |
| siRNA 1978 | 1978 | CAGGCGGCUUGUGGCUCCC | 4029 | GGGAGCCACAAGCCGCCUG |
| siRNA 1979 | 1979 | AGGCGGCUUGUGGCUCCCU | 4030 | AGGGAGCCACAAGCCGCCU |
| siRNA 1980 | 1980 | GGCGGCUUGUGGCUCCCUC | 4031 | GAGGGAGCCACAAGCCGCC |
| siRNA 1981 | 1981 | GCGGCUUGUGGCUCCCUCC | 4032 | GGAGGGAGCCACAAGCCGC |
| siRNA 1982 | 1982 | CGGCUUGUGGCUCCCUCCU | 4033 | AGGAGGGAGCCACAAGCCG |
| siRNA 1983 | 1983 | GGCUUGUGGCUCCCUCCUG | 4034 | CAGGAGGGAGCCACAAGCC |
| SiRNA 1984 | 1984 | GCUUGUGGCUCCCUCCUGA | 4035 | UCAGGAGGGAGCCACAAGC |
| siRNA 1985 | 1985 | CUUGUGGCUCCCUCCUGAC | 4036 | GUCAGGAGGGAGCCACAAG |
| siRNA 1986 | 1986 | UUGUGGCUCCCUCCUGACC | 4037 | GGUCAGGAGGGAGCCACAA |
| siRNA 1987 | 1987 | UGUGGCUCCCUCCUGACCC | 4038 | GGGUCAGGAGGGAGCCACA |
| siRNA 1988 | 1988 | GUGGCUCCCUCCUGACCCU | 4039 | AGGGUCAGGAGGGAGCCAC |
| siRNA 1989 | 1989 | UGGCUCCCUCCUGACCCUC | 4040 | GAGGGUCAGGAGGGAGCCA |
| siRNA 1990 | 1990 | GGCUCCCUCCUGACCCUCC | 4041 | GGAGGGUCAGGAGGGAGCC |
| siRNA 1991 | 1991 | GCUCCCUCCUGACCCUCCA | 4042 | UGGAGGGUCAGGAGGGAGC |
| siRNA 1992 | 1992 | CUCCCUCCUGACCCUCCAG | 4043 | CUGGAGGGUCAGGAGGGAG |
| siRNA 1993 | 1993 | UCCCUCCUGACCCUCCAGC | 4044 | GCUGGAGGGUCAGGAGGGA |
| siRNA 1994 | 1994 | CCCUCCUGACCCUCCAGCG | 4045 | CGCUGGAGGGUCAGGAGGG |
| siRNA 1995 | 1995 | CCUCCUGACCCUCCAGCGG | 4046 | CCGCUGGAGGGUCAGGAGG |
| siRNA 1996 | 1996 | CUCCUGACCCUCCAGCGGG | 4047 | CCCGCUGGAGGGUCAGGAG |
| siRNA 1997 | 1997 | UCCUGACCCUCCAGCGGGA | 4048 | UCCCGCUGGAGGGUCAGGA |
| siRNA 1998 | 1998 | CCUGACCCUCCAGCGGGAC | 4049 | GUCCCGCUGGAGGGUCAGG |
| siRNA 1999 | 1999 | CUGACCCUCCAGCGGGACA | 4050 | UGUCCCGCUGGAGGGUCAG |
| siRNA 2000 | 2000 | UGACCCUCCAGCGGGACAC | 4051 | GUGUCCCGCUGGAGGGUCA |
| siRNA 2001 | 2001 | GACCCUCCAGCGGGACACC | 4052 | GGUGUCCCGCUGGAGGGUC |
| siRNA 2002 | 2002 | ACCCUCCAGCGGGACACCC | 4053 | GGGUGUCCCGCUGGAGGGU |
| siRNA 2003 | 2003 | CCCUCCAGCGGGACACCCU | 4054 | AGGGUGUCCCGCUGGAGGG |
| siRNA 2004 | 2004 | CCUCCAGCGGGACACCCUG | 4055 | CAGGGUGUCCCGCUGGAGG |
| siRNA 2005 | 2005 | CUCCAGCGGGACACCCUGG | 4056 | CCAGGGUGUCCCGCUGGAG |
| siRNA 2006 | 2006 | UCCAGCGGGACACCCUGGU | 4057 | ACCAGGGUGUCCCGCUGGA |
| siRNA 2007 | 2007 | CCAGCGGGACACCCUGGUU | 4058 | AACCAGGGUGUCCCGCUGG |
| siRNA 2008 | 2008 | CAGCGGGACACCCUGGUUC | 4059 | GAACCAGGGUGUCCCGCUG |
| siRNA 2009 | 2009 | AGCGGGACACCCUGGUUCC | 4060 | GGAACCAGGGUGUCCCGCU |
| siRNA 2010 | 2010 | GCGGGACACCCUGGUUCCC | 4061 | GGGAACCAGGGUGUCCCGC |
| siRNA 2011 | 2011 | CGGGACACCCUGGUUCCCA | 4062 | UGGGAACCAGGGUGUCCCG |
| siRNA 2012 | 2012 | GGGACACCCUGGUUCCCAC | 4063 | GUGGGAACCAGGGUGUCCC |
| siRNA 2013 | 2013 | GGACACCCUGGUUCCCACC | 4064 | GGUGGGAACCAGGGUGUCC |
| siRNA 2014 | 2014 | GACACCCUGGUUCCCACCA | 4065 | UGGUGGGAACCAGGGUGUC |
| siRNA 2015 | 2015 | ACACCCUGGUUCCCACCAU | 4066 | AUGGUGGGAACCAGGGUGU |
| siRNA 2016 | 2016 | CACCCUGGUUCCCACCAUU | 4067 | AAUGGUGGGAACCAGGGUG |
| siRNA 2017 | 2017 | ACCCUGGUUCCCACCAUUC | 4068 | GAAUGGUGGGAACCAGGGU |
| siRNA 2018 | 2018 | CCCUGGUUCCCACCAUUCC | 4069 | GGAAUGGUGGGAACCAGGG |
| siRNA 2019 | 2019 | CCUGGUUCCCACCAUUCCC | 4070 | GGGAAUGGUGGGAACCAGG |
| siRNA 2020 | 2020 | CUGGUUCCCACCAUUCCCU | 4071 | AGGGAAUGGUGGGAACCAG |
| siRNA 2021 | 2021 | UGGUUCCCACCAUUCCCUG | 4072 | CAGGGAAUGGUGGGAACCA |
| siRNA 2022 | 2022 | GGUUCCCACCAUUCCCUGC | 4073 | GCAGGGAAUGGUGGGAACC |
| siRNA 2023 | 2023 | GUUCCCACCAUUCCCUGCC | 4074 | GGCAGGGAAUGGUGGGAAC |
| siRNA 2024 | 2024 | UUCCCACCAUUCCCUGCCU | 4075 | AGGCAGGGAAUGGUGGGAA |
| siRNA 2025 | 2025 | UCCCACCAUUCCCUGCCUU | 4076 | AAGGCAGGGAAUGGUGGGA |
| siRNA 2026 | 2026 | CCCACCAUUCCCUGCCUUG | 4077 | CAAGGCAGGGAAUGGUGGG |
| siRNA 2027 | 2027 | CCACCAUUCCCUGCCUUGC | 4078 | GCAAGGCAGGGAAUGGUGG |
| siRNA 2028 | 2028 | CACCAUUCCCUGCCUUGCU | 4079 | AGCAAGGCAGGGAAUGGUG |
| siRNA 2029 | 2029 | ACCAUUCCCUGCCUUGCUG | 4080 | CAGCAAGGCAGGGAAUGGU |
| siRNA 2030 | 2030 | CCAUUCCCUGCCUUGCUGA | 4081 | UCAGCAAGGCAGGGAAUGG |
| siRNA 2031 | 2031 | CAUUCCCUGCCUUGCUGAC | 4082 | GUCAGCAAGGCAGGGAAUG |
| siRNA 2032 | 2032 | AUUCCCUGCCUUGCUGACA | 4083 | UGUCAGCAAGGCAGGGAAU |
| siRNA 2033 | 2033 | UUCCCUGCCUUGCUGACAA | 4084 | UUGUCAGCAAGGCAGGGAA |
| siRNA 2034 | 2034 | UCCCUGCCUUGCUGACAAU | 4085 | AUUGUCAGCAAGGCAGGGA |
| siRNA 2035 | 2035 | CCCUGCCUUGCUGACAAUA | 4086 | UAUUGUCAGCAAGGCAGGG |
| siRNA 2036 | 2036 | CCUGCCUUGCUGACAAUAA | 4087 | UUAUUGUCAGCAAGGCAGG |
| siRNA 2037 | 2037 | CUGCCUUGCUGACAAUAAA | 4088 | UUUAUUGUCAGCAAGGCAG |
| siRNA 2038 | 2038 | UGCCUUGCUGACAAUAAAG | 4089 | CUUUAUUGUCAGCAAGGCA |
| siRNA 2039 | 2039 | GCCUUGCUGACAAUAAAGA | 4090 | UCUUUAUUGUCAGCAAGGC |
| siRNA 2040 | 2040 | CCUUGCUGACAAUAAAGAU | 4091 | AUCUUUAUUGUCAGCAAGG |
| siRNA 2041 | 2041 | CUUGCUGACAAUAAAGAUA | 4092 | UAUCUUUAUUGUCAGCAAG |
| siRNA 2042 | 2042 | UUGCUGACAAUAAAGAUAU | 4093 | AUAUCUUUAUUGUCAGCAA |
| siRNA 2043 | 2043 | UGCUGACAAUAAAGAUAUU | 4094 | AAUAUCUUUAUUGUCAGCA |
| SiRNA 2044 | 2044 | GCUGACAAUAAAGAUAUUU | 4095 | AAAUAUCUUUAUUGUCAGC |
| siRNA 2045 | 2045 | CUGACAAUAAAGAUAUUUC | 4096 | GAAAUAUCUUUAUUGUCAG |
| siRNA 2046 | 2046 | UGACAAUAAAGAUAUUUCC | 4097 | GGAAAUAUCUUUAUUGUCA |
| siRNA 2047 | 2047 | GACAAUAAAGAUAUUUCCA | 4098 | UGGAAAUAUCUUUAUUGUC |
| siRNA 2048 | 2048 | ACAAUAAAGAUAUUUCCAA | 4099 | UUGGAAAUAUCUUUAUUGU |
| SiRNA 2049 | 2049 | CAAUAAAGAUAUUUCCAAG | 4100 | CUUGGAAAUAUCUUUAUUG |
| siRNA 2050 | 2050 | AAUAAAGAUAUUUCCAAGA | 4101 | UCUUGGAAAUAUCUUUAUU |
| siRNA 2051 | 2051 | AUAAAGAUAUUUCCAAGAA | 4102 | UUCUUGGAAAUAUCUUUAU |

Further Sequences

| SEQ ID | |
|---|---|
| NO: | 5′ to 3′ Sequence |
| 4803 | GCCTCCCACTGCCCCTCAGGCCAGCTCAGGAGCCA |
| | TGGGGCGCTGGGCCTGGGTCCCCAGCCCCTGGCCC |
| | CCACCGGGGCTGGGCCCCTTCCTCCTCCTCCTCCT |
| | GCTGCTGCTGCTGCTGCCACGGGGGTTCCAGCCCC |
| | AGCCTGGCGGGAACCGTACGGAGTCCCCAGAACCT |
| | AATGCCACAGCGACCCCTGCGATCCCCACTATCCT |
| | GGTGACCTCTGTGACCTCTGAGACCCCAGCAACAA |
| | GTGCTCCAGAGGCAGAGGGACCCCAAAGTGGGGGG |
| | CTCCCGCCCCCGCCCAGGGCAGTTCCCTCGAGCAG |
| | TAGCCCCCAGGCCCAAGCACTCACCGAGGACGGGA |
| | GGCCCTGCAGGTTCCCCTTCCGCTACGGGGGCCGC |
| | ATGCTGCATGCCTGCACTTCGGAGGGCAGTGCACA |
| | CAGGAAGTGGTGTGCCACAACTCACAACTACGACC |
| | GGGACAGGGCCTGGGGCTACTGTGTGGAGGCCACC |
| | CCGCCTCCAGGGGGCCCAGCTGCCCTGGATCCCTG |
| | TGCCTCCGGCCCCTGCCTCAATGGAGGCTCCTGCT |
| | CCAATACCCAGGACCCCCAGTCCTATCACTGCAGC |
| | TGCCCCCGGGCCTTCACCGGCAAGGACTGCGGCAC |
| | AGAGAAATGCTTTGATGAGACCCGCTACGAGTACC |
| | TGGAGGGGGGCGACCGCTGGGCCCGCGTGCGCCAG |
| | GGCCACGTGGAACAGTGCGAGTGCTTCGGGGGCCG |
| | GACCTGGTGCGAAGGCACCCGACATACAGCTTGTC |
| | TGAGCAGCCCTTGCCTGAACGGGGGCACCTGCCAC |
| | CTGATCGTGGCCACCGGGACCACCGTGTGTGCCTG |
| | CCCACCAGGCTTCGCTGGACGGCTCTGCAACATCG |
| | AGCCTGATGAGCGCTGCTTCTTGGGGAACGGCACT |
| | GGGTACCGTGGCGTGGCCAGCACCTCAGCCTCGGG |
| | CCTCAGCTGCCTGGCCTGGAACTCCGATCTGCTCT |
| | ACCAGGAGCTGCACGTGGACTCCGTGGGCGCCGCG |
| | GCCCTGCTGGGCCTGGGCCCCCATGCCTACTGCCG |
| | GAATCCGGACAATGACGAGAGGCCCTGGTGCTACG |
| | TGGTGAAGGACAGCGCGCTCTCCTGGGAGTACTGC |
| | CGCCTGGAGGCCTGCGAATCCCTCACCAGAGTCCA |
| | ACTGTCACCGGATCTCCTGGCGACCCTGCCTGAGC |
| | CAGCCTCCCCGGGGCGCCAGGCCTGCGGCAGGAGG |
| | CACAAGAAGAGGACGTTCCTGCGGCCACGTATCAT |
| | CGGCGGCTCCTCCTCGCTGCCCGGCTCGCACCCCT |
| | GGCTGGCCGCCATCTACATCGGGGACAGCTTCTGC |
| | GCCGGGAGCCTGGTCCACACCTGCTGGGTGGTGTC |
| | GGCCGCCCACTGCTTCTCCCACAGCCCCCCCAGGG |
| | ACAGCGTCTCCGTGGTGCTGGGCCAGCACTTCTTC |
| | AACCGCACGACGGACGTGACGCAGACCTTCGGCAT |
| | CGAGAAGTACATCCCGTACACCCTGTACTCGGTGT |
| | TCAACCCCAGCGACCACGACCTCGTCCTGATCCGG |
| | CTGAAGAAGAAAGGGGACCGCTGTGCCACACGCTC |
| | GCAGTTCGTGCAGCCCATCTGCCTGCCCGAGCCCG |
| | GCAGCACCTTCCCCGCAGGACACAAGTGCCAGATT |
| | GCGGGCTGGGGCCACTTGGATGAGAACGTGAGCGG |
| | CTACTCCAGCTCCCTGCGGGAGGCCCTGGTCCCCC |
| | TGGTCGCCGACCACAAGTGCAGCAGCCCTGAGGTC |
| | TACGGCGCCGACATCAGCCCCAACATGCTCTGTGC |
| | CGGCTACTTCGACTGCAAGTCCGACGCCTGCCAGG |
| | GGGACTCAGGGGGGCCCCTGGCCTGCGAGAAGAAC |
| | GGCGTGGCTTACCTCTACGGCATCATCAGCTGGGG |
| | TGACGGCTGCGGGCGGCTCCACAAGCCGGGGGTCT |
| | ACACCCGCGTGGCCAACTATGTGGACTGGATCAAC |
| | GACCGGATACGGCCTCCCAGGCGGCTTGTGGCTCC |
| | CTCCTGACCCTCCAGCGGGACACCCTGGTTCCCAC |
| | CATTCCCTGCCTTGCTGACAATAAAGATATTTCCA |
| | AGAA |
| 4804 | [ETL17]scuaTmgUfGfGfAfcuggmaucaaasusu |
| 4805 | [ETL17]scuaTmgUfGfGfAfcugmgaucaaasusu |
| 4806 | [ETL17]scuauGfuGfgAfcuggaucaaasusu |
| 4807 | [ETL17]scuauGfuGfgAfcuggaucaaasusu |
| 4808 | [ETL17]scuauGfuGfgAfcuggaucaaasusu |
| 4809 | [ETL17]scuauGfuGfgAfcuggaucaaasusu |
| 4810 | [ETL17]scuauGfuGfgAfcuggaucaaasusu |
| 4811 | [ETL17]scuauGfuGfgAfcuggaucaaasusu |
| 4812 | [ETL17]scuauGfuGfgAfcuggaucaaasusu |
| 4813 | [ETL17]scuauGfuGfgAfcuggaucaaasusu |
| 4814 | usUfsugaUfcCfaGfuCfcAfcAfuAfgsusu |
| 4815 | usUfsugaUfcCfaGfuccAfcAfuAfgsusu |
| 4816 | usUfsugaUfcCfaGfuccAfcAfuagsusu |
| 4817 | usUfsugaUfCfcaGfuCfcAfcAfuAfgsusu |
| 4818 | usUfsugauCfcaGfuCfcAfcAfuAfgsusu |
| 4819 | usUfsugAfuCfcaGfuCfcAfcAfuAfgsusu |
| 4820 | usUfsugAfuCfcaGfuccAfcAfuAfgsusu |
| 4821 | usUfsugAfuCfcAfguccAfcAfuagsusu |

What is claimed is:

1. A composition comprising an oligonucleotide that targets hepatocyte growth factor activator (HGFAC) and when administered to a subject having cancer in an effective amount improves a clinical response related to the cancer.

2. The composition of claim 1, wherein the improved clinical response comprises at least a 10% increase in a clinical response measurement relative to a baseline clinical response measurement obtained from the subject prior to administration of the composition.

3. The composition of claim 1, wherein the clinical response comprises progression free survival, duration of response, disease control rate, health-related quality of life, milestone survival, clinical benefit rate, pathological complete response, complete response, objective response rate, duration of clinical benefit, time to next treatment, time to treatment failure, disease-free survival, or time to cancer progression.

4. A composition comprising an oligonucleotide that targets HGFAC and when administered to a subject in an effective amount alters an immune cell measurement in a subject.

5. The composition of claim 4, wherein the immune cell measurement is altered by about 10% or more, as compared to prior to administration.

6. The composition of claim 4, wherein the immune cell measurement comprises a myeloid derived suppressor cell or subpopulation count, CD8+ tumor infiltrating lymphocyte count, leukocyte count, T lymphocyte count, activated T lymphocyte count, B lymphocyte count, activated B lymphocyte count, monocyte count, macrophage count, activated macrophage count, dendritic cell count, neutrophil count, eosinophil count, basophil count, or mast cell count.

7. A composition comprising an oligonucleotide that targets HGFAC and when administered to a subject in an effective amount increases an antibody level in a subject.

8. The composition of claim 7, wherein the antibody level is increased by about 10% or more, as compared to prior to administration.

9. The composition of claim 7, wherein the antibody level comprises an IgA level, IgG level, or IgM level.

10. A composition comprising an oligonucleotide that targets HGFAC and when administered to a subject in an effective amount decreases a tumor marker level in a subject.

11. The composition of claim 10, wherein the tumor marker level is decreased by about 10% or more, as compared to prior to administration.

12. The composition of claim 10, wherein the tumor marker comprises CEA, PSA, CA 125, CA 15-3, CA 19-9, CA 27.29, CA 72-4, AFP, hCG, B2M, BTA, Calcitonin, CgA, CELLSEARCH, DCP, Gastrin, HE4, LDH, NSE, NMP22, or PAP.

13. The composition of any one of claims 1-12, wherein the oligonucleotide comprises a modified internucleoside linkage.

14. The composition of claim 13, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

15. The composition of claim 13, wherein the modified internucleoside linkage comprises one or more phosphorothioate linkages.

16. The composition of any one of claims 1-12, wherein the oligonucleotide comprises 1, 2, 3, 4, 5, 6, 7, 8, 9, 10, 11, 12, 13, 14, 15, 16, 17, 18, 19, or 20 modified internucleoside linkages.

17. The composition of any one of claims 1-12, wherein the oligonucleotide comprises a modified nucleoside.

18. The composition of claim 17, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

19. The composition of claim 17, wherein the modified nucleoside comprises a LNA.

20. The composition of claim 17, wherein the modified nucleoside comprises a 2′,4′ constrained ethyl nucleic acid.

21. The composition of claim 17, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

22. The composition of claim 17, wherein the modified nucleoside comprises one or more 2′fluoro modified nucleosides.

23. The composition of claim 17, wherein the modified nucleoside comprises a 2′ O-alkyl modified nucleoside.

24. The composition of any one of claims 1-12, wherein the oligonucleotide comprises 1, 2, 3, 4, 5, 6, 7, 8, 9, 10, 11, 12, 13, 14, 15, 16, 17, 18, 19, 20, or 21 modified nucleosides.

25. The composition of any one of claims 1-12, wherein the oligonucleotide comprises a lipid attached at a 3′ or 5′ terminus of the oligonucleotide.

26. The composition of claim 25, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

27. The composition of any one of claims 1-12, wherein the oligonucleotide comprises a sugar moiety attached at a 3′ or 5′ terminus of the oligonucleotide.

28. The composition of claim 27, wherein the sugar comprises N-acetylgalactosamine (GalNAc), N-acetylglucosamine (GlcNAc), or mannose.

29. The composition of any one of claims 1-12, wherein the oligonucleotide comprises a small interfering RNA (siRNA) comprising a sense strand and an antisense strand.

30. The composition of claim 29, wherein the sense strand is 12-30 nucleosides in length.

31. The composition of claim 29, wherein the antisense strand is 12-30 nucleosides in length.

32. A composition comprising an oligonucleotide that inhibits the expression of HGFAC, wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 4803.

33. The composition of claim 29, wherein any one of the following is true with regard to the sense strand:
all purines comprise 2′ fluoro modified purines, and all pyrimidines comprise a mixture of 2′ fluoro and 2′-O-methyl modified pyrimidines;
all purines comprise 2′-O-methyl modified purines, and all pyrimidines comprise a mixture of 2′ fluoro and 2′-O-methyl modified pyrimidines;
all purines comprise 2′ fluoro modified purines, and all pyrimidines comprise 2′-O-methyl modified pyrimidines;
all pyrimidines comprise 2′ fluoro modified pyrimidines, and all purines comprise a mixture of 2′ fluoro and 2′-O-methyl modified purines;
all pyrimidines comprise 2′-O-methyl modified pyrimidines, and all purines comprise a mixture of 2′ fluoro and 2′-O-methyl modified purines; or
all pyrimidines comprise 2′ fluoro modified pyrimidines, and all purines comprise 2′-O-methyl modified purines.

34. The composition of claim 29, wherein any one of the following is true with regard to the antisense strand:
all purines comprise 2′ fluoro modified purines, and all pyrimidines comprise a mixture of 2′ fluoro and 2′-O-methyl modified pyrimidines;
all purines comprise 2′-O-methyl modified purines, and all pyrimidines comprise a mixture of 2′ fluoro and 2′-O-methyl modified pyrimidines;
all purines comprise 2′-O-methyl modified purines, and all pyrimidines comprise 2′ fluoro modified pyrimidines;
all pyrimidines comprise 2′ fluoro modified pyrimidines, and all purines comprise a mixture of 2′ fluoro and 2′-O-methyl modified purines;
all pyrimidines comprise 2′-O-methyl modified pyrimidines, and all purines comprise a mixture of 2′ fluoro and 2′-O-methyl modified purines; or
all pyrimidines comprise 2′-O-methyl modified pyrimidines, and all purines comprise 2′ fluoro modified purines.

35. The composition of any one of claims 1-12, wherein the oligonucleotide comprises an antisense oligonucleotide (ASO).

36. The composition of claim 35, wherein the ASO is 12-30 nucleosides in length.

37. A composition comprising an oligonucleotide that inhibits the expression of HGFAC, wherein the oligonucleotide comprises an ASO about 12-30 nucleosides in length and a nucleoside sequence complementary to about 12-30 contiguous nucleosides of SEQ ID NO: 4803.

38. The composition of any one of claims 1-12, further comprising a pharmaceutically acceptable carrier.

39. A method of treating a subject having cancer, comprising administering an effective amount of the composition of any one of claims 1-12 to the subject.

40. The method of claim 39, further comprising administering a checkpoint inhibitor to the subject.

41. The method of claim 40, wherein the checkpoint inhibitor comprises a PD1 inhibitor, a PD11inhibitor, a CTLA4 inhibitor of a combination thereof.

42. The method of claim 39, further comprising administering radiotherapy to the subject.

43. The method of claim 39, wherein the cancer comprises a malignant neoplasm, a solid tumor, or a hematological cancer.

44. The method of claim 39, wherein the cancer comprises a malignant neoplasm of a urinary tract, malignant neoplasm of an endocrine gland, malignant neoplasm of a soft tissue, malignant neoplasm of skin, malignant neoplasm of a skeletal system, malignant neoplasm of a respiratory organ, malignant neoplasm of an intrathoracic organ, malignant neoplasm of a genital organ, malignant neoplasm of a lip, malignant neoplasm of an oral cavity, malignant neoplasm of a pharynx, malignant neoplasm of an eye, malignant neoplasm of a central nervous system, malignant neoplasm of a brain, malignant neoplasm of a digestive system, malignant neoplasm of a breast, malignant neoplasm of a pancreas, or a malignant melanoma.

45. A method of treating cancer in a subject in need thereof, the method comprising administering the subject an effective amount of a composition that inhibits HGFAC.

46. The method of claim 45, wherein the composition comprises an oligonucleotide.

47. The method of claim 46, wherein the oligonucleotide comprises an siRNA.

48. A method of treating cancer in a subject in need thereof, the method comprising administering the subject an effective amount of an siRNA that targets HGFAC.

49. The method of claim 48, wherein the administration reduces a symptom or a clinical finding associated with the cancer by at least 10%.
